(19) United States
(12) Patent Application Publication
(10) Pub. No.: US 2025/0011316 A1
(43) Pub. Date: Jan. 9, 2025

(54) PEROXIREDOXIN 3 INHIBITORS AND METHODS OF USE FOR TREATING CANCER

(71) Applicant: Wake Forest University Health Sciences, Winston-Salem, NC (US)

(72) Inventors: W. Todd Lowther, Pfafftown, NC (US); Terrence L. Smalley, JR., Greensboro, NC (US); Kimberly J. Nelson, Clemmons, NC (US)

(21) Appl. No.: 18/811,956

(22) Filed: Aug. 22, 2024

Related U.S. Application Data
(63) Continuation-in-part of application No. PCT/US2023/072904, filed on Aug. 25, 2023.
(60) Provisional application No. 63/373,626, filed on Aug. 26, 2022.

(51) Int. Cl. C07D 417/04; A61K 31/426; A61K 31/427; A61K 31/4439; A61K 31/454; A61K 31/496; A61K 31/5377; A61K 38/00; A61K 47/55; C07D 277/56; C07D 417/12; C07D 417/14; C07K 7/06

(52) U.S. Cl. CPC C07D 417/04; A61K 31/426; A61K 31/427; A61K 31/4439; A61K 31/454; A61K 31/496; A61K 31/5377; A61K 47/55; C07D 277/56; C07D 417/12; C07D 417/14; C07K 7/06; A61K 38/00

(57) ABSTRACT

Provided according to some embodiments is a compound of Formula (I), or a pharmaceutically acceptable salt or prodrug thereof. Pharmaceutical compositions comprising the same and methods of use for treating cancer and inhibiting PRX3 are also provided.

### CROSS REFERENCE TO RELATED APPLICATIONS

This application is a continuation-in-part of PCT/US2023/072904, filed Aug. 25, 2023, which claims priority to U.S. provisional application No. 63/373,626, filed Aug. 26, 2022. These applications are hereby incorporated by reference in their entireties.

### STATEMENT REGARDING FEDERALLY SPONSORED RESEARCH OR DEVELOPMENT

This invention was made with government support under Contract Number R01 GM072866 awarded by the National Institutes of Health. The U.S government has certain rights in the invention.

### BACKGROUND

A central hallmark of the tumorigeneses of cells is metabolic changes that cause an increase in the level of reactive oxygen species (ROS) and mitochondrial ROS (mROS). Cairns et al. (2011) Nat Rev Cancer 11, 85-95; Weinberg et al. (2009) Cell Mol Life Sci 66, 3663-3673; Weinberg et al. (2009) Ann N Y Acad Sci 1177, 66-73; Weinberg et al. (2010) Proc Natl Acad Sci USA 107, 8788-8793. In response to this neoplastic transformation, cells reorganize their antioxidant capacity to survive, proliferate and metastasize. Specifically, oncogene-induced increases in ROS levels activate the oncogenic transcription factor FOXM1, inducing the expression of FOXM1 target genes including the mitochondrial antioxidant enzymes superoxide dismutase 2 and peroxiredoxin 3 (PRX3). Park et al. (2009) EMBO J 28, 2908-2918; Nonn et al. (2003) Mol Cancer Res 1, 682-689.

PRX3 is a peroxidase responsible for metabolizing ˜90% f mitochondrial hydrogen peroxide (H₂O₂) (Cox et al. (2009) Biochem J 425, 313-325), and this specific ROS is known to regulate several important processes involved in tumor progression including proliferation, apoptosis, migration and metastasis. The GEPIA2 database of matched pairs of patient samples (tumor vs. normal) illustrates how the PRX3 transcript levels are elevated in 15/32 (46.9%) of the tumor tissues collected, including many forms of cancer with significant unmet medical need. Tang et al. (2019) Nucleic Acids Res 47, W556-W560. PRX3 protein expression and mROS levels correlate with sensitivity to the natural product and PRX3 inhibitor thiostrepton (TS) in patient-derived malignant mesothelioma cells lines. Nelson et al. (2021) Antioxidants (Basel) 10, 150.

PRX3 expression supports malignant mesothelioma (MM) and ovarian tumor (OvCa) cell growth. Cunniff et al. (2015) PloS one 10, e0127310; Myers (2016) Free Radic Biol Med 91, 81-92; Yoshikawa et al. (2016) Oncol Rep 35, 2543-2552; Wang et al. (2013) Tumour Biol 34, 2275-2281. PRX3 expression levels in OvCa and cervical cancer also correlate with poor patient outcomes. Li et al. (2018) Biosci Rep 38. The following additional features further support PRX3 as a promising molecular target for cancer therapy: (i) no cancer mutations in the PRX3 gene known to support resistance development; (ii) PRX3 KO mice are viable and reach maturity; increase in basal oxidative stress levels observed only in a variety of challenge models (Li et al. (2007) Biochem Biophys Res Commun 355, 715-721; Lee (2020) Antioxidants (Basel) 9); and (iii) partial knockdown of PRX3 via shRNA slows tumor cell proliferation and significantly reduced the expression of FOXM1 at the RNA and protein levels (Cunniff et al. (2015) PloS one 10, e0127310).

A study by Corsello et al. (Nature Cancer 2020 1(2):235-248) tested the ability of 4,518 drugs from the Drug Repurposing Hub at the Broad Institute to kill 578 cancer cells lines. Thiostrepton (TS), an insoluble, thiopeptide antibiotic, showed meaningful efficacy in 403 tumor cells lines derived from a wide array of tissues. Our team has demonstrated that TS acts by irreversibly crosslinking the two essential catalytic cysteine residues in PRX3, inactivating peroxidase activity and increasing ROS to levels incompatible with survival. Nelson et al. (2021) Antioxidants (Basel) 10, 150; Cunniff et al. (2015) PloS one 10, e0127310; Newick et al. (2012) PloS one 7, e39404. Because this irreversible crosslink occurs across the homodimer interface, the inactivated PRX3 is significantly larger in mass, and we can track the PRX3 crosslink in our cellular and animal models.

Several mechanisms have been proposed for TS cytotoxicity of cancer cells: (i) interaction with the oncogenic transcription factor FOXM1 (Hegde et al. (2011) Nat Chem 3, 725-731) (ii) inhibition of the 20/26S proteasome (Bhat et al. (2009) PloS one 4, e6593; Bird et al. (2020) ACS Chem Biol 15, 2164-2174), (iii) binding to the large subunit of ribosomes (Zhang et al. (2005) Antibiotic susceptibility of mammalian mitochondrial translation. FEBS Lett 579, 6423-6427; Harms et al. (2008) Mol Cell 30, 26-38), and (iv) covalent adduction and cross-linking of PRX3 by our team (Nelson et al. (2021) Antioxidants (Basel) 10, 150; Cunniff et al. (2015) PloS one 10, e0127310). We have shown that TS sensitivity is greatly decreased upon knockdown of PRX3 in a cell model of MM, indicating that PRX3 inhibition is key in driving TS cytotoxicity. Inhibition of PRX3 also significantly increases mitochondrial ROS which drives TS-mediated cell death. Increased ROS modulates FOXM1 expression while increased production of mitochondrial ROS has also been shown to disassemble 26S proteasome complexes (Livnat-Levanon et al. (2014) Cell Rep 7, 1371-1380; Segref et al. (2014) Cell Metab 19, 642-652), further complicating the interpretation of the mode of action of TS.

Despite the effectiveness of TS in cell and animal models of cancer, this natural product has serious limitations to its utility as a chemotherapy. First, it is highly large, highly insoluble, and does not exhibit any of the preferred drug like properties. Second, this molecule is currently produced by bacterial fermentation followed by organic extraction and purification. Although a synthetic route for synthesis has been published, it involved multiple steps, is expensive, and has low yield. Ayida et al. (2005) Bioorg Med Chem Lett 15, 2457-2460.

Improved PRX3 inhibitors that can address some of these issues are needed.

### SUMMARY

Provided herein according to some embodiments is a compound of Formula I

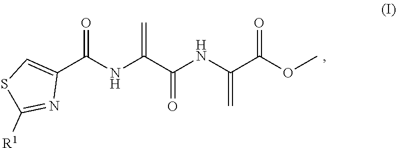

wherein R1 is an aryl, heteroaryl, cycloalkyl or heterocycle, which aryl, heteroaryl, cycloalkyl or heterocycle is optionally substituted with one or more selected from alkyl, carboxy, carbamate, urea, amide, amino, ether, ester and halo, or a pharmaceutically acceptable salt or prodrug thereof. In some embodiments, when R1 is pyridine or pyrazine, said pyridine or pyrazine is substituted with one or more selected from alkyl, carboxy, carbamate, urea, amide, amino, ether, ester and halo.

In some embodiments, the aryl, heteroaryl, cycloalkyl or heterocycle, is substituted with one or more selected from alkyl, carboxy, carbamate, urea, amide, and halo. In some embodiments, the aryl, heteroaryl, cycloalkyl or heterocycle, is substituted with carbamate or amide. In some embodiments, the aryl, heteroaryl, cycloalkyl or heterocycle, is substituted with an alkylcarbamate.

In some embodiments, R¹ is a group having a structure of

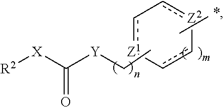

wherein:
n is 0, 1, 2 or 3;
m is 0, 1 or 2;
X is absent or is O, NR³, or CH₂;
Y is absent or is O, NR³, or CH₂;
Z¹ and Z² are each independently O, N, or C;
R² is alkyl (e.g., having from 1 to 8 carbon atoms, linear or branched), wherein said alkyl is optionally substituted (e.g., with halo, amino, ether, alkoxy, or carbamate), or heterocycle; and
R³ is H or alkyl (e.g., having from 1 to 8 carbon atoms, linear or branched),
wherein * denotes the connection of the group in the compound of Formula I.

In some embodiments, Z1 and Z2 are each independently N or C.

In some embodiments, R1 is a group having a structure of

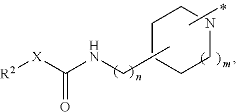

wherein:
n is 0, 1, 2 or 3;
m is 0, 1 or 2;
X is O or CH₂; and
R² is alkyl (e.g., having from 1 to 8 carbon atoms, linear or branched), wherein said alkyl is optionally substituted (e.g., with halo, amino, ether, alkoxy, or carbamate), or heterocycle, wherein * denotes the connection of the group in the compound of Formula I.

In some embodiments, R1 is a group having a structure of

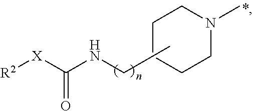

wherein:
n is 0, 1, 2 or 3;
X is O or CH2; and
R2 is alkyl (e.g., having from 1 to 8 carbon atoms, linear or branched), wherein said alkyl is optionally substituted (e.g., with halo, amino, ether, alkoxy, or carbamate), or heterocycle, wherein * denotes the connection of the group in the compound of Formula I, or a pharmaceutically acceptable salt or prodrug thereof.

In some embodiments, R1 is a group having a structure of

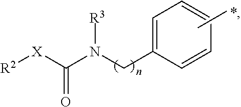

wherein:
n is 0, 1, 2 or 3;
X is O or CH₂;
R² is alkyl (e.g., having from 1 to 8 carbon atoms, linear or branched), wherein said alkyl is optionally substituted (e.g., with halo, amino, ether, alkoxy, or carbamate), or heterocycle; and
R³ is H or alkyl (e.g., having from 1 to 8 carbon atoms, linear or branched),
wherein * denotes the connection of the group in the compound of Formula I.

Also provided is a pharmaceutical composition comprising a compound or pharmaceutically acceptable salt or prodrug as taught herein. In some embodiments, the composition is formulated for oral or parenteral (e.g. intravenous, intrapleural, intraperitoneal or intraovarian) administration. In some embodiments, the composition is formulated for oral administration and is in the form of a capsule, cachet, lozenge, or tablet. In some embodiments, the formulation is provided in unit dosage form of from 1 mg to 10 grams of the compound, pharmaceutically acceptable salt or prodrug.

Further provided is a method treating cancer in a subject in need thereof, comprising administering to the subject a therapeutically effective amount of a compound of Formula I, or a pharmaceutically acceptable salt or prodrug thereof. Also provided is a compound of Formula I or a pharmaceutically acceptable salt or prodrug thereof for use in treating cancer in a subject in need thereof, or for preparing a medicament for use in treating cancer.

In some embodiments, the cancer has PRX3 expression.

In some embodiments, the subject is a human subject.

In some embodiments, the subject is a non-human animal subject (e.g. non-human mammalian subject).

In some embodiments, the administering is carried out by administering a pharmaceutical composition comprising said compound or pharmaceutically acceptable salt or prodrug.

In some embodiments, the administering further comprises administering bortezomib, carboplatin, paclitaxel, an immunotherapy agent, or a combination thereof.

In some embodiments, the administering further comprises administering doxorubicin.

Further provided is a method of inhibiting PRX3 in a subject in need thereof, comprising administering to said subject a therapeutically effective amount of a compound of Formula I, or a pharmaceutically acceptable salt or prodrug thereof.

### BRIEF DESCRIPTION OF THE FIGURES

FIG. 1: Cell viability assay data in Malignant Mesothelioma (MM) cells (HMESO cell line) treated with compound (1)—HCl or (5)—HCl N=2 technical and 2 biological replicates conducted by 2 different lab technicians. All compounds went into solution in 100% DMSO. (5)—HCl precipitated at concentrations of ˜20-80 μM when added to complete tissue culture media.

FIG. 2A: View (40× magnification) of HMESO mesothelioma cells treated with (5)—HCl.

FIG. 2B: View (20× magnification) of HMESO mesothelioma cells treated with (5)—HCl.

FIG. 3: Western blots of PRX3 from HMESO mesothelioma cells treated with (1) or (5).

FIG. 4: Bar graph of PRX3 (PRX3—X —PRX3): Monomer ratio of HMESO mesothelioma cells treated with (1) or (5).

FIG. 5 is a table summarizing the results of Examples 42-44 for various compounds described herein. NT indicates “not tested.” For EC₅₀ values, “A” represents an EC₅₀ less than or equal to 5.0 μM, “B” represents an EC₅₀ from 5.1 μM to 10.0 μM, “C” represents an EC₅₀ from 10.1 μM to 50.0 μM, and “D” represents an EC₅₀ greater than 50.0 μM.

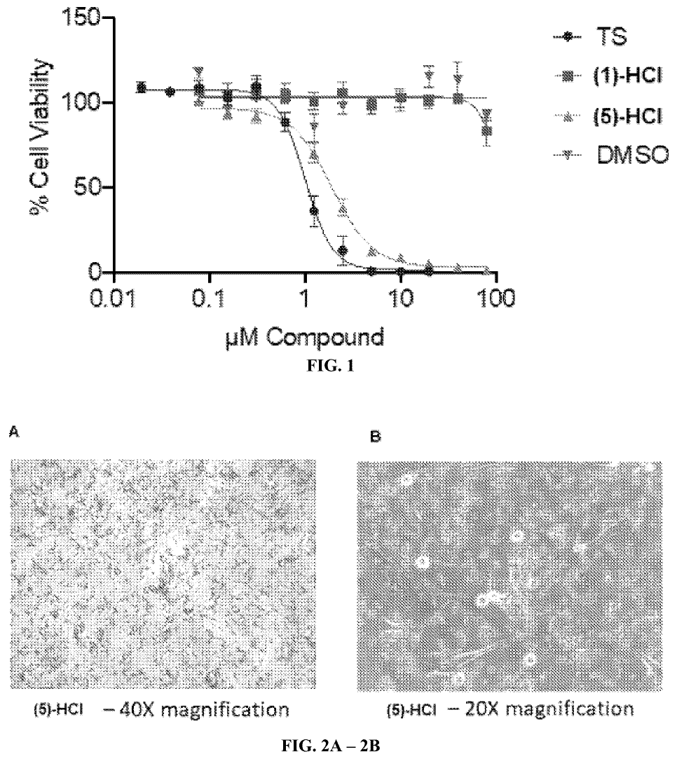

### DETAILED DESCRIPTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

The disclosures of all patent references cited herein are hereby incorporated by reference to the extent they are consistent with the disclosure set forth herein. As used herein in the description of the invention and the appended claims, the singular forms “a,” “an” and “the” are intended to include the plural forms as well, unless the context clearly indicates otherwise.

### Definitions

In general the terms and phrases used herein have their art-recognized meaning, which can be found by reference to standard texts, journal references and contexts known to those skilled in the art. The following definitions are provided to clarify their specific use in the context of the invention.

As used herein in the accompanying chemical structures, “H” refers to a hydrogen atom. “C” refers to a carbon atom. “N” refers to a nitrogen atom. “S” refers to a sulfur atom. “O” refers to an oxygen atom.

Unless indicated otherwise, nomenclature used to describe chemical groups or moieties as used herein follow the convention where, reading the name from left to right, the point of attachment to the rest of the molecule is at the right hand side of the name. For example, the group “alkylamino” is attached to the rest of the molecule at the amino end, whereas the group “aminoalkyl” is attached to the rest of the molecule at the alkyl end.

“Alkyl,” as used herein, refers to a saturated straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. “Lower alkyl” as used herein, is a subset of alkyl and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, tert-butyl, and the like. The alkyl groups may be optionally substituted with one or more suitable substituents, such as halo, hydroxy, carboxy, amine, etc.

“Cycloalkyl,” as used herein, refers to a saturated cyclic hydrocarbon containing from 1 to 10 carbon atoms. Representative examples of cycloalkyl include, but are not limited to, cyclopropyl, cyclobutyl, cyclopentyl, cyclohexyl, cycloheptyl, cyclooctyl, and the like. The cycloalkyl groups may be optionally substituted with one or more suitable substituents, such as halo, hydroxy, carboxy, amine, etc.

“Aryl,” as used herein, refers to a monocyclic carbocyclic ring system or a bicyclic carbocyclic fused or directly adjoining ring system having one or more aromatic rings. Examples include, but are not limited to, phenyl, indanyl, indenyl, tetrahydronaphthyl, biphenyl, napthyl, azulenyl, etc. The aryl may be optionally substituted with one or more suitable substituents, such as alkyl, halo, hydroxy, carboxy, amine, etc.

“Heteroaryl,” as used herein, refers to a monovalent aromatic group having a single ring or two fused or directly adjoining rings and containing in at least one of the rings at least one heteroatom (typically 1 to 3) independently selected from nitrogen, oxygen and sulfur. Examples include, but are not limited to, pyrrole, imidazole, thiazole, oxazole, furan, thiophene, triazole, pyrazole, isoxazole, isothiazole, pyridine, pyrazine, pyridazine, pyrimidine, triazine, benzothiophene, benzofuran, indole, benzimidazole, benzothiazole, quinoline, isoquinoline, quinazoline, quinoxaline, phenyl-pyrrole, phenyl-thiophene, etc. The heteroaryl may be optionally substituted with one or more suitable substituents, such as alkyl, halo, hydroxy, carboxy, amine, etc.

“Heterocycle” as used herein refers to a saturated or partially unsaturated cyclic hydrocarbon with at least one heteroatom (typically 1 to 3) independently selected from nitrogen, oxygen and sulfur. The heterocycle may be a monocyclic heterocycle, a bicyclic heterocycle, or a tricyclic heterocycle. The heterocycle may be optionally substituted with one or more suitable substituents, such as alkyl, halo, hydroxy, carboxy, amine, etc.

“Monocyclic heterocycle” means a 3—, 4-, 5-, 6-, 7-, or 8-membered ring containing at least one heteroatom, and which is not aromatic. Representative examples of monocyclic heterocycle include, but are not limited to, azetidinyl, azepanyl, aziridinyl, diazepanyl, 1,3-dioxanyl, 1,3-dioxolanyl, dihydropyranyl (including 3,4-dihydro-2H-pyran-6-yl), 1,3-dithiolanyl, 1,3-dithianyl, imidazolidinyl, isothiazolidinyl, isoxazolidinyl, morpholinyl, oxadiazolidinyl, oxazolidinyl, piperazinyl, piperidinyl, pyranyl, pyrazolidinyl, pyrrolidinyl, tetrahydrofuranyl, tetrahydropyranyl (including tetrahydro-2H-pyran-4-yl), tetrahydrothienyl, thiadiazolidinyl, thiazolidinyl, thiomorpholinyl, 1,1-dioxidothiomor-pholinyl (thiomorpholine sulfone), thiopyranyl, and trithianyl.

“Bicyclic heterocycle” means a monocyclic heterocycle fused to an aryl group, or a monocyclic heterocycle fused to a monocyclic cycloalkyl or cycloalkenyl, or a monocyclic heterocycle fused to a monocyclic heterocycle. Representative examples of bicyclic heterocycles include, but are not limited to, 3,4-dihydro-2H-pyranyl, 1,3-benzodioxolyl, 1,3-benzodithiolyl, 2,3-dihydro-1,4-benzodioxinyl, 2,3-dihydro-1-benzofuranyl, 2,3-dihydro-1-benzothienyl,2,3-dihydro-1H-indolyl, 3,4-dihydroquinolin-2(1H)-one and 1,2,3,4-tetrahydroquinolinyl.

“Tricyclic heterocycle” means a bicyclic heterocycle fused to an aryl group, or a bicyclic heterocycle fused to a monocyclic cycloalkyl or cycloalkenyl, or a bicyclic heterocycle fused to a monocyclic heterocycle. Representative examples of tricyclic heterocycles include, but are not limited to, 2,3,4,4a,9,9a-hexahydro-1H-carbazolyl, 5a,6,7,8,9,9a-hexahydro-dibenzo[b,d]furanyl, and 5a,6,7,8,9,9a-hexahydrodibenzo[b,d]thienyl.

The terms “halo” and “halogen” refer to fluoro (—F), choro (—Cl), bromo (—Br), or iodo (—I).

“Haloalkyl” refers to one or more halo groups appended to the parent molecular moiety through an alkyl group. Examples include, but are not limited to, chloromethyl, fluoromethyl, trifluoromethyl, etc.

“Carboxy” refers to the group —COOH.

“Alkoxy” refers to an alkyl or cycloalkyl group, as herein defined, attached to the principal carbon chain through an oxygen atom. Representative examples of “alkoxy” include, but are not limited to, methoxy, ethoxy, propoxy, 2-propoxy, butoxy, tert-butoxy, and hexyloxy.

“Hydroxy” or “hydroxyl” refers to an —OH group.

An “amine” or “amino” refers to a group —NH2, wherein none, one or two of the hydrogens may be replaced by an alkyl, cycloalkyl, or aryl as defined herein.

An “amide” or “amido” refers to a group having a carbonyl bonded to a nitrogen atom, such as —C(O)NH2, wherein none, one or two of the hydrogens may be replaced by an alkyl, cycloalkyl, heterocycle, or aryl as defined herein.

An “ether” refers to a group in which there is an ether, R—O—R′, wherein R and R′ are each independently an alkyl, cycloalkyl, or aryl as defined herein.

An “ester” refers to a group in which there is an ester, R—C(O)—O—R′, wherein R and R′ are each independently an alkyl, cycloalkyl, or aryl as defined herein.

A “carbamate” refers to a group in which there is a carbamate, R—O—C(O)NR′R”, wherein R, R′ and R” are each independently an alkyl, cycloalkyl, or aryl as defined herein.

A “urea” refers to a group in which there is a urea, R—NH—C(O)—NH—R′, wherein R and R′ are each independently an alkyl, cycloalkyl, or aryl as defined herein.

As understood in the art, the term “optionally substituted” indicates that the specified group is either unsubstituted, or substituted by one or more suitable substituents. A “substituent” that is “substituted” is a group which takes the place of one or more hydrogen atoms on the parent organic molecule.

Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (b) salts formed from elemental anions such as chlorine, bromine, and iodine.

### Active Compounds

Active compounds useful as PRX3 inhibitors in accordance with the present invention are provided below. Unless otherwise stated, structures depicted herein are also meant to include all enantiomeric, diastereomeric, and geometric (or conformational) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Tautomeric forms include keto-enol tautomers of a compound. In addition, unless otherwise stated, all rotamer forms of the compounds of the invention are within the scope of the invention.

Unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a 13C- or 14C-enriched carbon are within the scope of this invention. Such compounds are useful, for example, as analytical tools or probes in biological assays.

Provided herein according to some embodiments as an active compound is a compound of Formula I

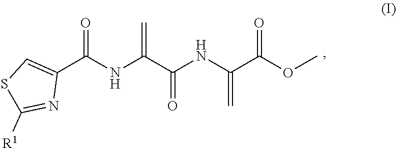

wherein R1 is an aryl, heteroaryl, cycloalkyl or heterocycle, which aryl, heteroaryl, cycloalkyl or heterocycle is optionally substituted with one or more selected from alkyl, carboxy, carbamate, urea, amide, amino, ether, ester and halo, or a pharmaceutically acceptable salt or prodrug thereof.

In some embodiments, when R1 is pyridine or pyrazine, said pyridine or pyrazine is substituted with one or more selected from alkyl, carboxy, carbamate, urea, amide, amino, ether, ester and halo.

In some embodiments, the aryl, heteroaryl, cycloalkyl or heterocycle, is substituted with one or more selected from alkyl, carboxy, carbamate, urea, amide, and halo.

In some embodiments, the aryl, heteroaryl, cycloalkyl or heterocycle, is substituted with carbamate or amide.

In some embodiments, the aryl, heteroaryl, cycloalkyl or heterocycle, is substituted with an alkylcarbamate.

In some embodiments, R1 is a group having a structure of

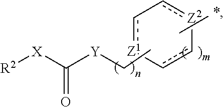

wherein:
n is 0, 1, 2 or 3;
m is 0, 1 or 2;
X is absent or is O, NR3, or CH₂;
Y is absent or is O, NR3, or CH₂;
Z1 and Z2 are each independently O, N, or C;
R2 is alkyl (e.g., having from 1 to 8 carbon atoms, linear or branched), wherein said alkyl is optionally substituted (e.g., with halo, amino, ether, alkoxy, or carbamate), or heterocycle; and
R3 is H or alkyl (e.g., having from 1 to 8 carbon atoms, linear or branched),
wherein * denotes the connection of the group in the compound of Formula I (i.e., the connection of the group at the 2-position of the thiazole ring).

In some embodiments, Z1 and Z2 are each independently N or C.

Particular examples of active compounds include, but are not limited to, those selected from the group consisting of

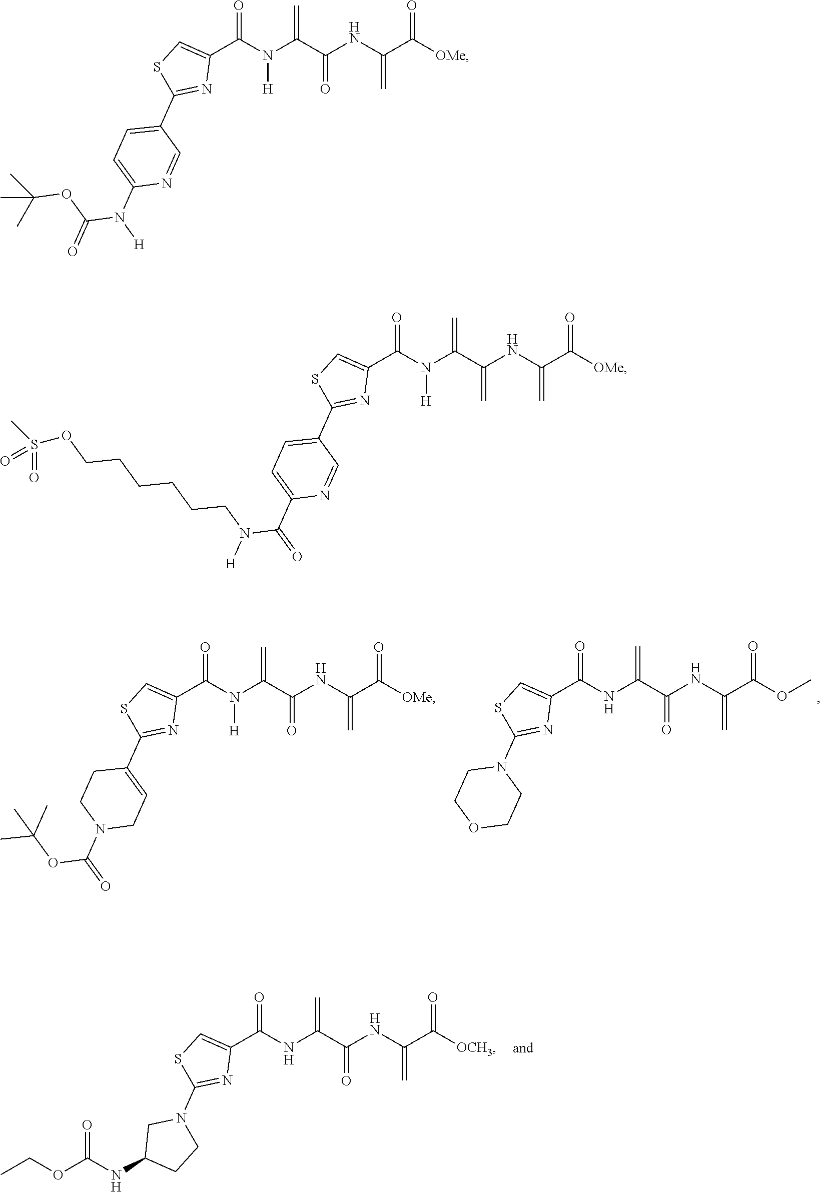 [SMILES: C=C(NC(=O)c1csc(-c2ccc(NC(=O)OC(C)(C)C)nc2)n1)C(=O)NC(=C)C(=O)OC ; C=C(NC(=C)C(=O)OC)C(=C)NC(=O)c1csc(-c2ccc(C(=O)NCCCCCCOS(C)(=O)=O)nc2)n1 ; C=C(NC(=O)c1csc(C2=CCN(C(=O)OC(C)(C)C)CC2)n1)C(=O)NC(=C)C(=O)OC ; C=C(NC(=O)c1csc(N2CCOCC2)n1)C(=O)NC(=C)C(=O)OC ; C=C(NC(=O)c1csc(N2CC[C@@H](NC(=O)OCC)C2)n1)C(=O)NC(=C)C(=O)OC]

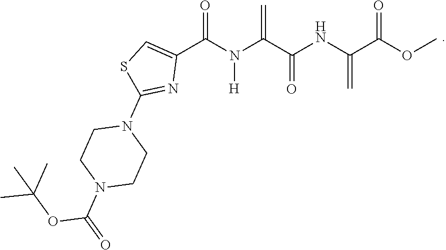 [SMILES: C=C(NC(=O)c1csc(N2CCN(C(=O)OC(C)(C)C)CC2)n1)C(=O)NC(=C)C(=O)OC]

In some embodiments, R1 is a group having a structure of

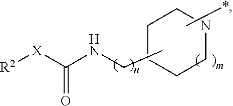

wherein:
n is 0, 1, 2 or 3;
m is 0, 1 or 2;
X is O or CH₂; and
R2 is alkyl (e.g., having from 1 to 8 carbon atoms, linear or branched), wherein said alkyl is optionally substituted (e.g., with halo, amino, ether, alkoxy, or carbamate), or heterocycle; and
wherein * denotes the connection of the group in the compound of Formula I (i.e., the connection of the group at the 2-position of the thiazole ring).

In some embodiments, R1 is a group having a structure of

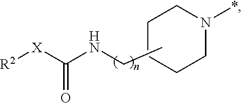

wherein:
n is 0, 1, 2 or 3;
X is O or CH2; and
R2 is alkyl (e.g., having from 1 to 8 carbon atoms, linear or branched), wherein said alkyl is optionally substituted (e.g., with halo, amino, ether, alkoxy, or carbamate), or heterocycle; and
wherein * denotes the connection of the group in the compound of Formula I (i.e., the connection of the group at the 2-position of the thiazole ring).

Particular examples of active compounds include, but are not limited to, those selected from the group consisting of:

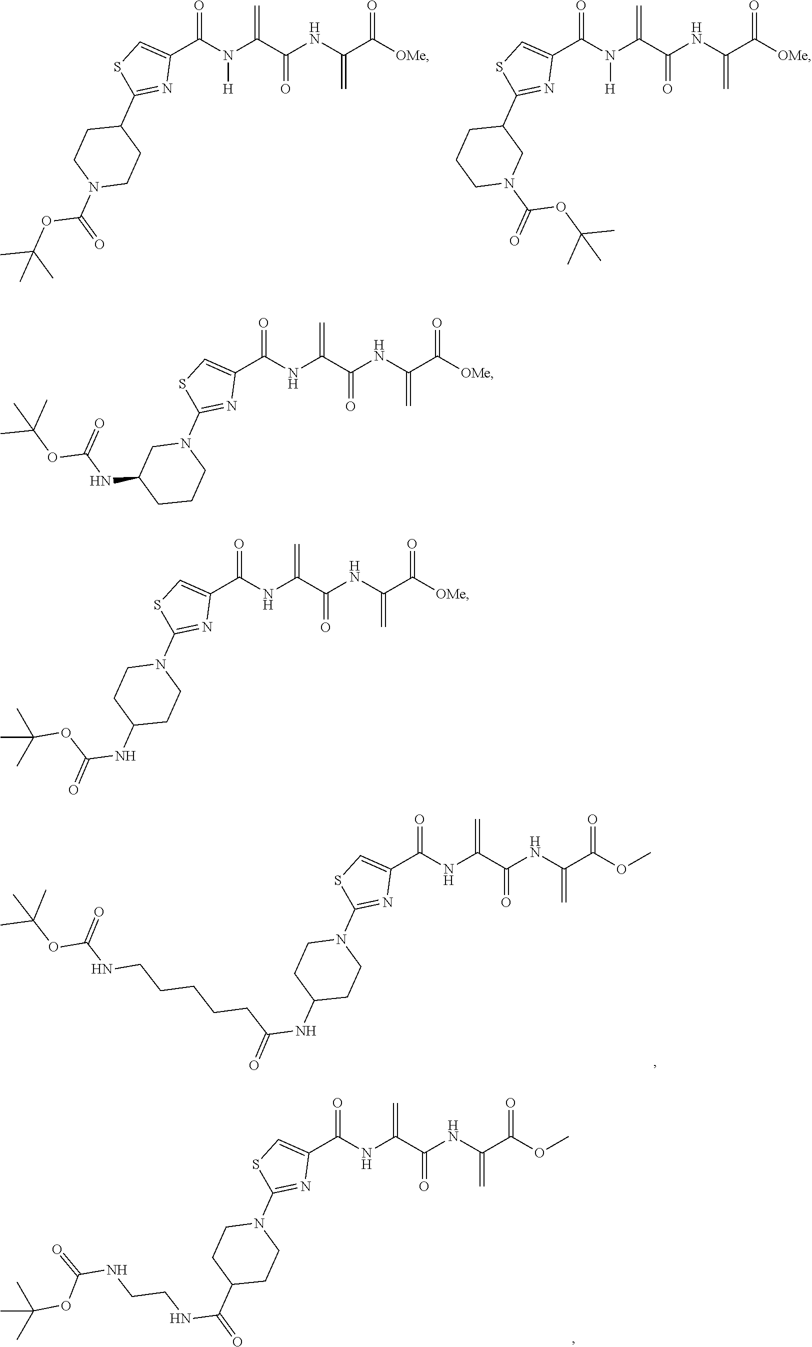

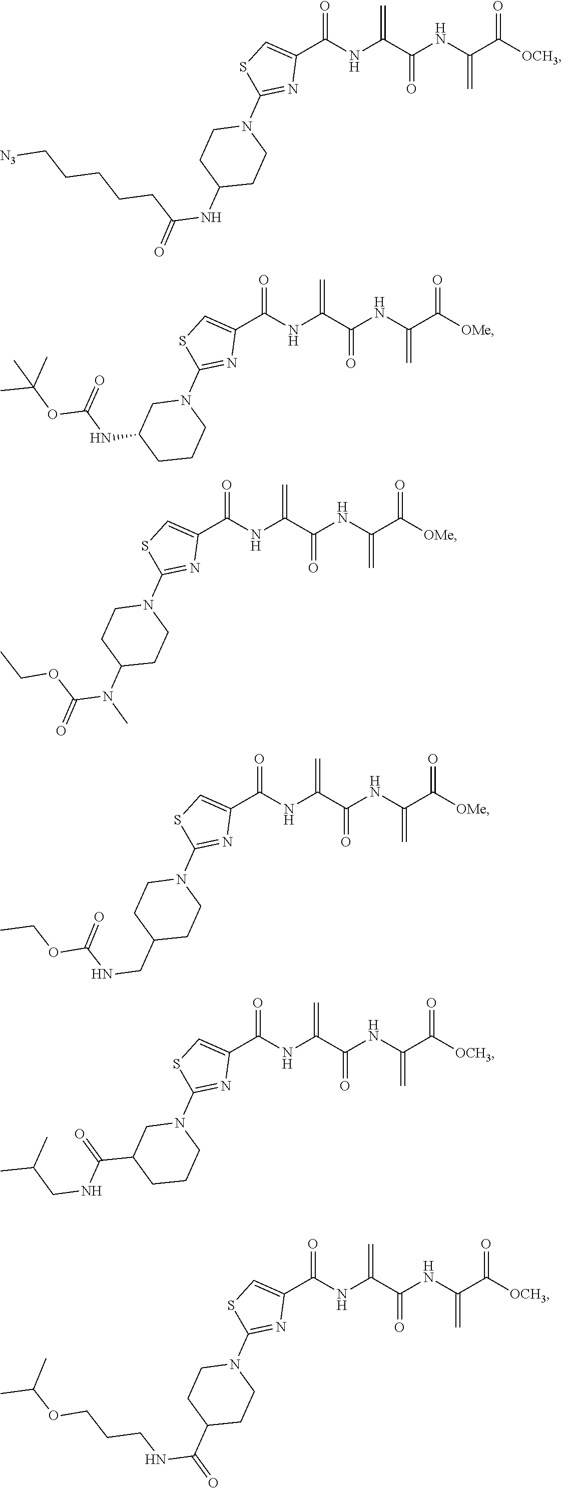

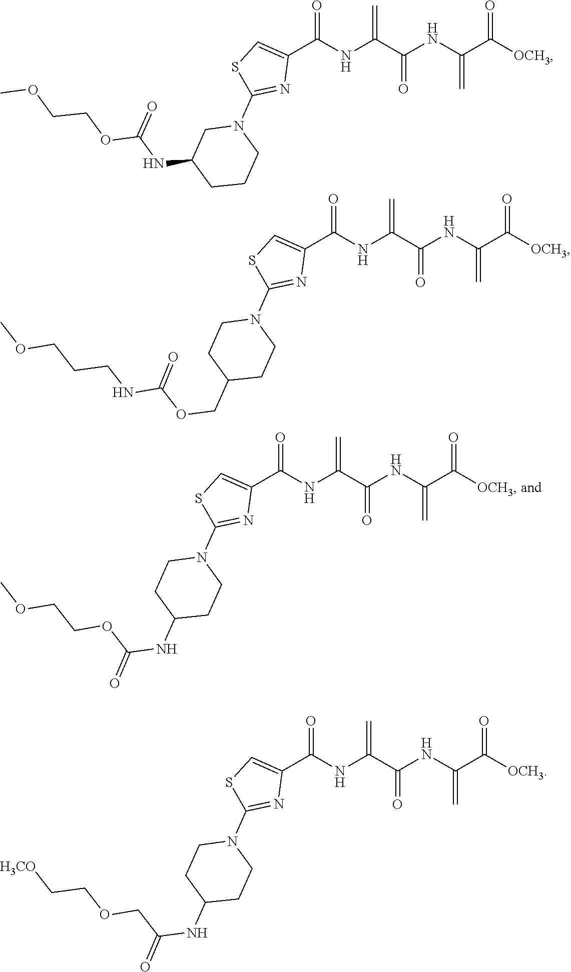

In some embodiments, R¹ is a group having a structure of

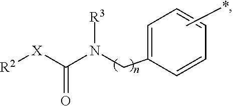

wherein:
n is 0, 1, 2 or 3;
X is O or CH₂;
R² is alkyl (e.g., having from 1 to 8 carbon atoms, linear or branched), wherein said alkyl is optionally substituted (e.g., with halo, amino, ether, alkoxy, or carbamate), or heterocycle; and
R³ is H or alkyl (e.g., having from 1 to 8 carbon atoms, linear or branched),
wherein * denotes the connection of the group in the compound of Formula I (i.e., the connection of the group at the 2-position of the thiazole ring).

Particular examples of active compounds include, but are not limited to, those selected from the group consisting of

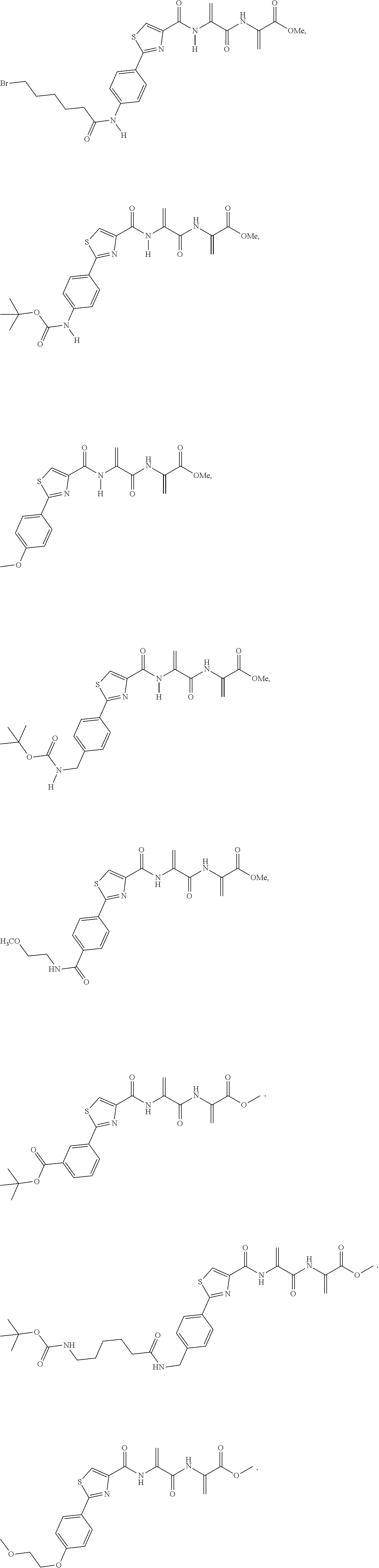

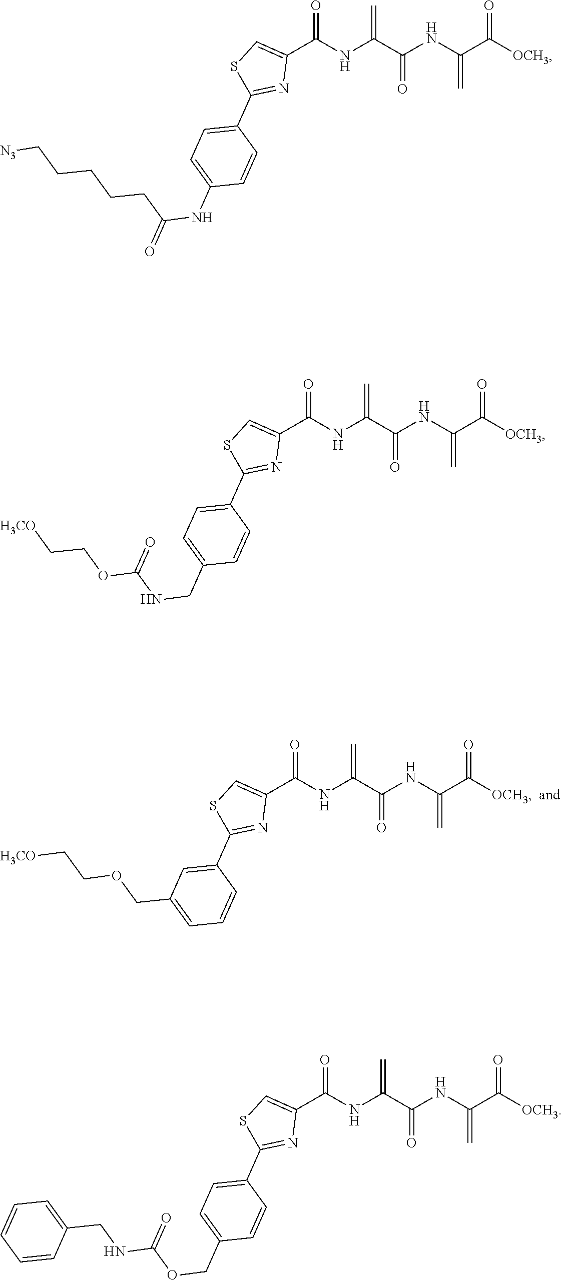

Particular examples of active compounds include, but are not limited to, those selected from the group consisting of

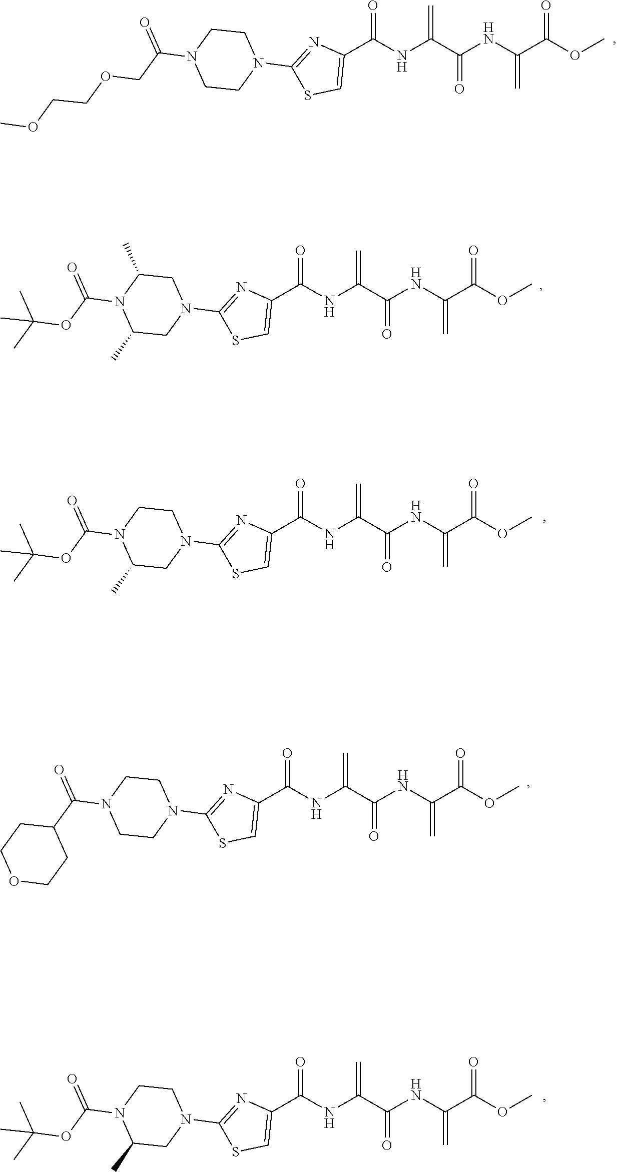

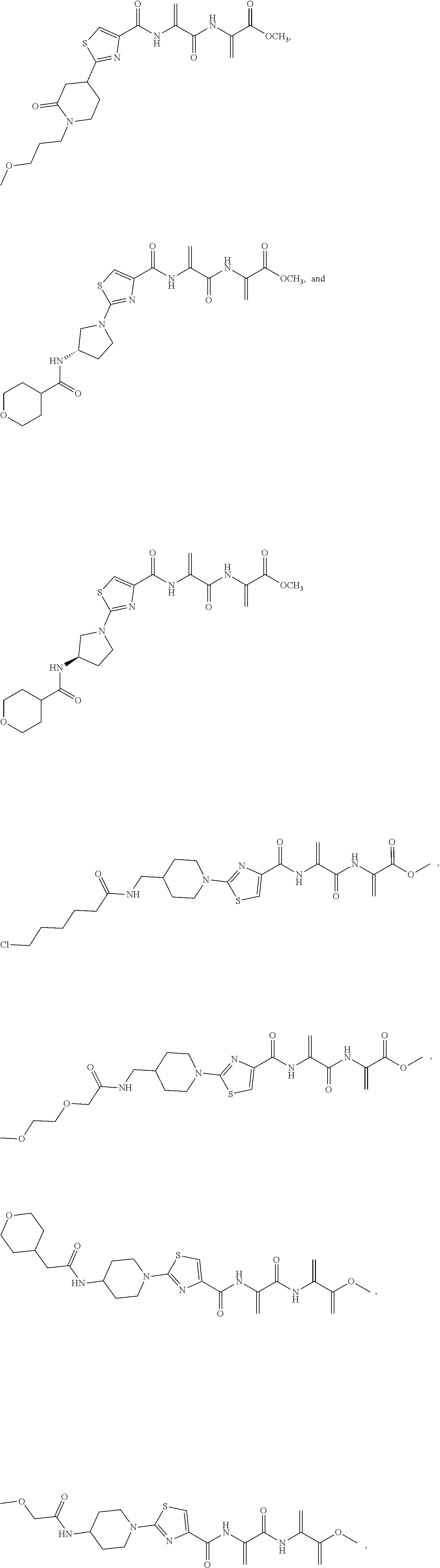

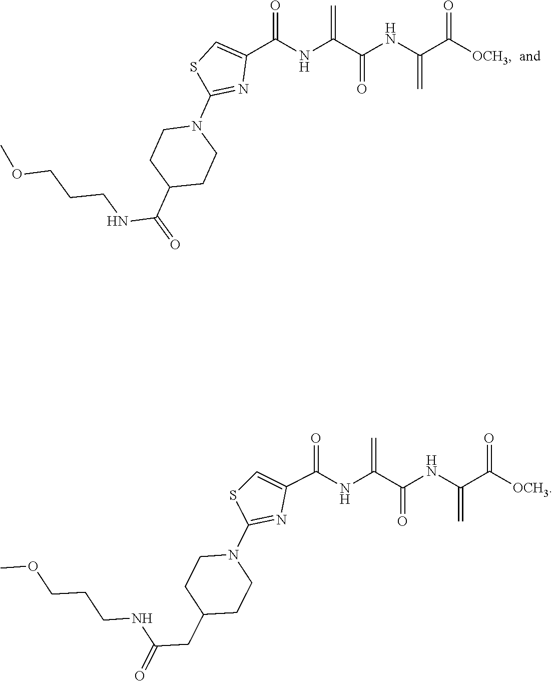

Particular examples of active compounds include, but are not limited to, those selected from the group consisting of

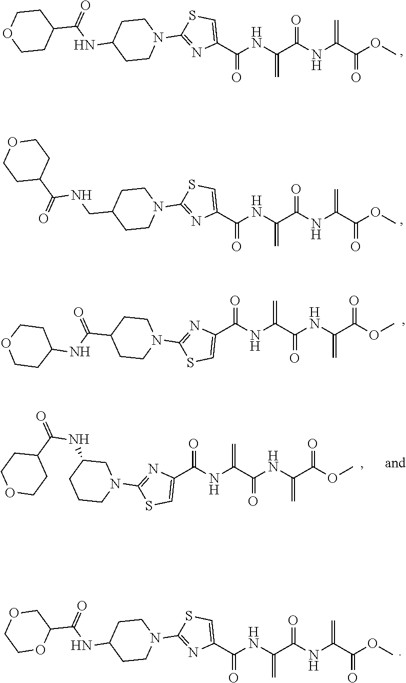 [SMILES: C=C(NC(=O)c1csc(N2CCC(NC(=O)C3CCOCC3)CC2)n1)C(=O)NC(=C)C(=O)OC ; C=C(NC(=O)c1csc(N2CCC(CNC(=O)C3CCOCC3)CC2)n1)C(=O)NC(=C)C(=O)OC ; C=C(NC(=O)c1csc(N2CCC(C(=O)NC3CCOCC3)CC2)n1)C(=O)NC(=C)C(=O)OC ; C=C(NC(=O)c1csc(N2CCC[C@H](NC(=O)C3CCOCC3)C2)n1)C(=O)NC(=C)C(=O)OC ; C=C(NC(=O)c1csc(N2CCC(NC(=O)C3COCCO3)CC2)n1)C(=O)NC(=C)C(=O)OC]

Particular examples of active compounds include, but are not limited to, those selected from the group consisting of

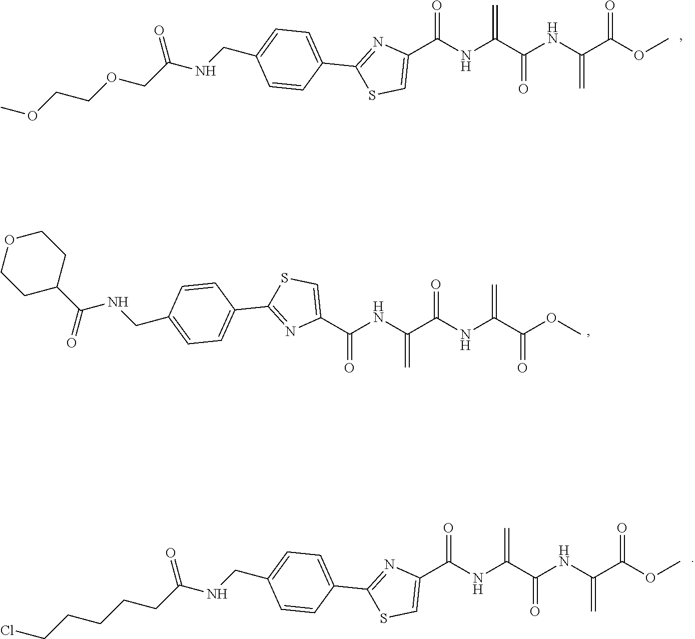

Particular examples of active compounds include, but are not limited to, those selected from the group consisting of:

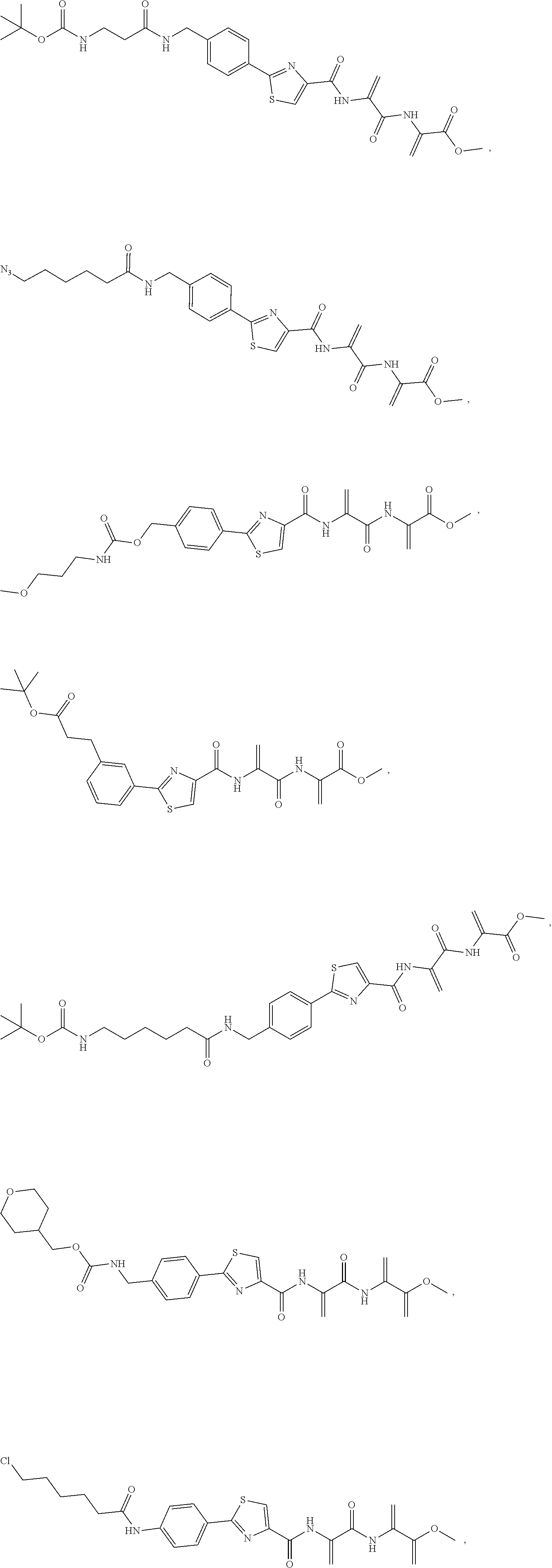

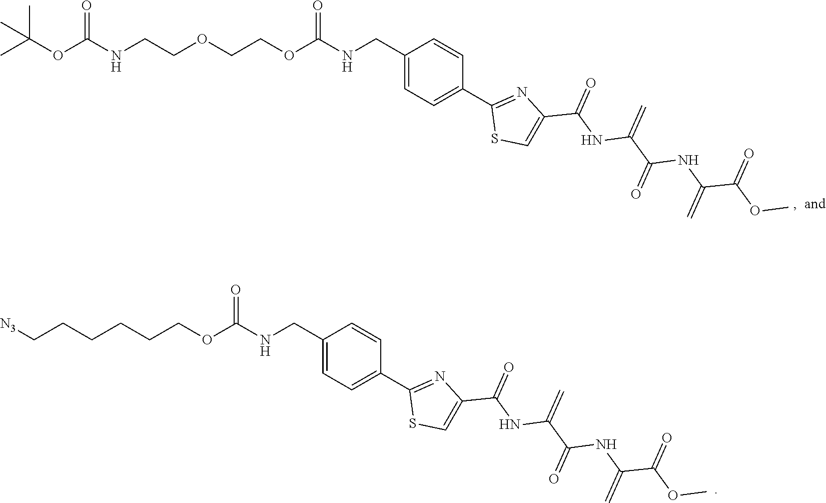 [SMILES: C=C(NC(=O)c1csc(-c2ccc(CNC(=O)OCCOCCNC(=O)OC(C)(C)C)cc2)n1)C(=O)NC(=C)C(=O)OC ; C=C(NC(=O)c1csc(-c2ccc(CNC(=O)OCCCCCCN=[N+]=[N-])cc2)n1)C(=O)NC(=C)C(=O)OC]

In some embodiments, an active compound can form a covalent adduct with PRX3 in a biochemical PRX3 inhibition assay, which may support its PRX3 inhibition activity.

In some embodiments, an active compound can have an EC₅₀ in a cellular activity assay (such as the ability to kill cancer cells such as SKOV3 ovarian cancer cells) in the micromolar range, such as from 0.05, 0.1, 0.25, or 0.5 micromolar, to 10, 15, or 20 micromolar.

In some embodiments, an active compound can have good solubility, e.g., solubility in aqueous solution (e.g., saline such as phosphate buffered saline, water, etc.) of at least 0.1 millimolar, such as from 0.1 to 1 millimolar, or solubility in an aqueous solution of at least 1 millimolar.

In some embodiments, an active compound can have good aqueous stability. For example, in some embodiments, an active compound may have no decrease in purity after 24 hours in an aqueous solution.

In some embodiments, an active compound does not have appreciable antimicrobial activity, e.g., at greater than 20 micromolar concentrations.

In some embodiments, an active compound does not inhibit the proteasome and/or does not inhibit FOXM1 DNA binding. These may indicate that the compound has greater specificity for PRX3 than TS.

### Methods of Use

The terms “treat”, “treatment” and “treating” as used herein refer to any type of treatment that imparts a benefit to a subject afflicted with a disease or disorder, delay in the progression of the disease or disorder, or symptoms thereof, etc. In some embodiments, the treatment is for a cancer (e.g., a cancer having elevated reactive oxygen species).

In some embodiments, the subject treated is a human subject. In some embodiments, the subject is a non-human animal (e.g., non-human mammalian subject). A non-human animal may include, but is not limited to, non-human primates, dogs, cats, horses, cattle, goats, pigs, sheep, guinea pigs, mice, rats and rabbits, as well as any other domestic, commercially or clinically valuable animal, including but not limited to animal models and livestock animals. In some embodiments, the subject is a subject in need of a treatment such as a treatment of the present invention.

Cancers that may be treated with the active compounds according to some embodiments may include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bile duct cancer; bladder cancer; bone cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cardiac tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ductal carcinoma in situ; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; multiple myeloma; heavy chain disease (e.g., alpha chain disease, gamma chain disease, mu chain disease); hemangioblastoma; histiocytosis; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); melanoma; midline tract carcinoma; multiple endocrine neoplasia syndrome; muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); nasopharynx cancer; neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); parathryroid cancer; papillary adenocarcinoma; penile cancer (e.g., Paget's disease of the penis and scrotum); pharyngeal cancer; pinealoma; pituitary cancer; pleuropulmonary blastoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; retinoblastoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; stomach cancer; small intestine cancer; sweat gland carcinoma; synovioma; testicular cancer (e.g., seminoma, testicular embryonal carcinoma); thymic cancer; thyroid cancer (e.g., papillary carcinoma of the thyroid, papillary thyroid carcinoma (PTC), medullary thyroid cancer); urethral cancer; uterine cancer; vaginal cancer; and vulvar cancer (e.g., Paget's disease of the vulva). See also US 2019/0153098 to Goldberg et al.

In some embodiments, the cancer is a blood cancer such as leukemia, liver cancer, lung cancer, lymphoma, melanoma, prostate cancer, head and neck cancer, bladder cancer, brain cancer, breast cancer, or cervical cancer. In some embodiments, the cancer is prostate cancer. In some embodiments, the cancer is head and neck cancer. In some embodiments, the cancer is ovarian cancer. In some embodiments, the cancer is cervical cancer. In some embodiments, the cancer is malignant mesothelioma.

In some embodiments, the cancer has PRX3 expression. For example, the cancer may be a cancer type generally known to express PRX3 and/or the cancer has been determined (e.g., by testing a biopsy) to have PRX3 expression.

The cancer may be metastatic, in which cancerous cells from a primary or original tumor migrate to another organ or tissue and may be identified as the tissue type of the primary or original tumor and not of that of the organ or tissue in which the secondary (metastatic) tumor is located. As a non-limiting example, a prostate cancer that has migrated to bone is said to be metastasized prostate cancer and includes cancerous prostate cancer cells growing in bone tissue.

### Pharmaceutical Formulations

The active compounds disclosed herein can, as noted above, be prepared in the form of their pharmaceutically acceptable salts. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

Active compounds of the present invention may be prepared as pharmaceutically acceptable prodrugs. Such prodrugs are those which are, within the scope of sound medical judgment, suitable for use in contact with the tissues of humans and lower animals without undue toxicity, irritation, allergic response and the like, commensurate with a reasonable risk/benefit ratio, and effective for their intended use, as well as the zwitterionic forms, where possible, of the compounds of the invention. The term “prodrug” refers to compounds that are rapidly transformed in vivo to yield the parent compound of the above formulae, for example, by hydrolysis in blood. A thorough discussion is provided in T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein. See also U.S. Pat. No. 6,680,299. Examples include a prodrug that is metabolized in vivo by a subject to an active drug having an activity of active compounds as described herein, wherein the prodrug is an ester of an alcohol or carboxylic acid group, if such a group is present in the compound; an acetal or ketal of an alcohol group, if such a group is present in the compound; an N-Mannich base or an imine of an amine group, if such a group is present in the compound; or a Schiff base, oxime, acetal, enol ester, oxazolidine, or thiazolidine of a carbonyl group, if such a group is present in the compound, such as described in U.S. Pat. Nos. 6,680,324 and 6,680,322.

The active compounds described above may be formulated for administration in a pharmaceutical carrier in accordance with known techniques. See, e.g., Remington, The Science and Practice of Pharmacy (9th Ed. 1995). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% r 99% by weight of the active compound. One or more active compounds may be incorporated in the formulations of the invention, which may be prepared by any of the well known techniques of pharmacy comprising admixing the components, optionally including one or more accessory ingredients.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature, severity and location of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the active compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the active compound(s), which preparations are preferably isotonic with the blood of the intended recipient. These preparations may contain antioxidants, buffers, bacteriostats and solutes which render the formulation isotonic with the blood of the intended recipient. Aqueous and non-aqueous sterile suspensions may include suspending agents and thickening agents. The formulations may be presented in unit/dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising an active compound(s), or a salt thereof, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. Suitable formulations comprise citrate or bis/tris buffer (pH 6) or ethanol/water and contain from 0.1 to 0.2M active ingredient.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer which forms the structure of the liposome. In either instance, the liposomes which are produced may be reduced in size, as through the use of standard sonication and homogenization techniques. The liposomal formulations containing the compounds disclosed herein or salts thereof may be lyophilized to produce a lyophilizate which may be reconstituted with a pharmaceutically acceptable carrier, such as water, to regenerate a liposomal suspension.

Other pharmaceutical compositions may be prepared from the compounds disclosed herein, or salts thereof, such as aqueous base emulsions. In such an instance, the composition will contain a sufficient amount of pharmaceutically acceptable emulsifying agent to emulsify the desired amount of the compound or salt thereof. Particularly useful emulsifying agents include phosphatidyl cholines, and lecithin.

In addition to active compound(s), the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. If desired, the pharmaceutical compositions of the present invention may be lyophilized using techniques well known in the art.

### DOSAGE AND ROUTES OF ADMINISTRATION

As noted above, the present invention provides pharmaceutical formulations comprising the active compounds (including the pharmaceutically acceptable salts thereof), in pharmaceutically acceptable carriers for oral, rectal, topical, buccal, parenteral, intrapleural, intraovarian, intramuscular, intradermal, intravascular, and/or transdermal administration. Parenteral administration may be, for example, intravascular (intravenous or intraarterial), intrapleural, intraperitoneal or intraovarian administration by injection, infusion or implantation.

The therapeutically effective dosage of any specific compound, the use of which is in the scope of present invention, will vary somewhat from compound to compound, and patient to patient, and will depend upon the condition of the patient and the route of delivery. As a general proposition, a dosage from about 0.1 to about 50 mg/kg is expected to have therapeutic efficacy, with all weights being calculated based upon the weight of the active compound, including the cases where a salt is employed. Toxicity concerns at the higher level may restrict intravenous dosages to a lower level such as up to about 10 mg/kg, with all weights being calculated based upon the weight of the active base, including the cases where a salt is employed. A dosage from about 10 mg/kg to about 50 mg/kg may be employed for oral administration. Typically, a dosage from about 0.5 mg/kg to 5 mg/kg may be employed for intramuscular injection.

Depending upon the condition being treated, the compounds described herein may be administered alone or concurrently with one or more additional active agent useful for treating the disease or condition with which the patient is afflicted. Examples of additional active agents include, but are not limited to, those set forth in paragraphs 0065 through 0387 of W. Hunter, D. Gravett, et al., US Patent Application Publication No. 20050181977 (Published Aug. 18, 2005) (assigned to Angiotech International AG) the disclosure of which is incorporated by reference herein in its entirety.

The present invention is explained in greater detail in the following non-limiting Examples.

### EXAMPLES

We have determined the minimal fragment of TS that covalently modifies, crosslinks, and inactivates PRX3. Moreover, compounds based on this fragment inhibit PRX3, can be specific for PRX3 over other human PRX isoforms, kill multiple cancer cell types in culture, and are effective in a mouse model of malignant mesothelioma.

### ABBREVIATIONS

mmole: millimoles
g: grams
mL: milliliters
HOBt.H2O: 1-hydroxybenzotriazole hydrate
EDC·HCl: 1-(3-dimethylaminopropyl)-3-ethylcarbodiimide hydrochloride
HCl: hydrochloric acid
DCM: dichloromethane or methylene chloride
DBU: 1,8-diazabicyclo[5.4.0]undec-7-ene
(i-Pr)₂NEt, DIPEA: N,N-diisopropylethylamine
NMM: 4-methylmorpholine
Et₃N: triethylamine
THF: tetrahydrofuran
TBAF: Tetrabutylammonium fluoride
DMA: N,N-Dimethylacetamide
4-DMAP: 4-Dimethylaminopyridine
TBAI: Tetrabutylammonium iodide
CDI: N,N′-Carbonyldiimidazole
DCC: N,N′-Dicyclohexylcarbodiimide
DME: 1,2-Dimethoxyethane
TFAA: Trifluoroacetic anhydride
LAH, LiAlH₄: Lithium aluminum hydride
TBTU: O-(Benzotriazol-1-yl)-N,N,N′,N′-tetramethyluronium tetrafluoroborate
HATU: 1-[Bis(dimethylamino)methylene]-1H-1,2,3-triazolo[4,5-b]pyridinium 3-oxide
hexafluorophosphate
DMF: N,N-Dimethylformamide
Aq.: aqueous
RT: room temperature

### General Synthetic Procedures

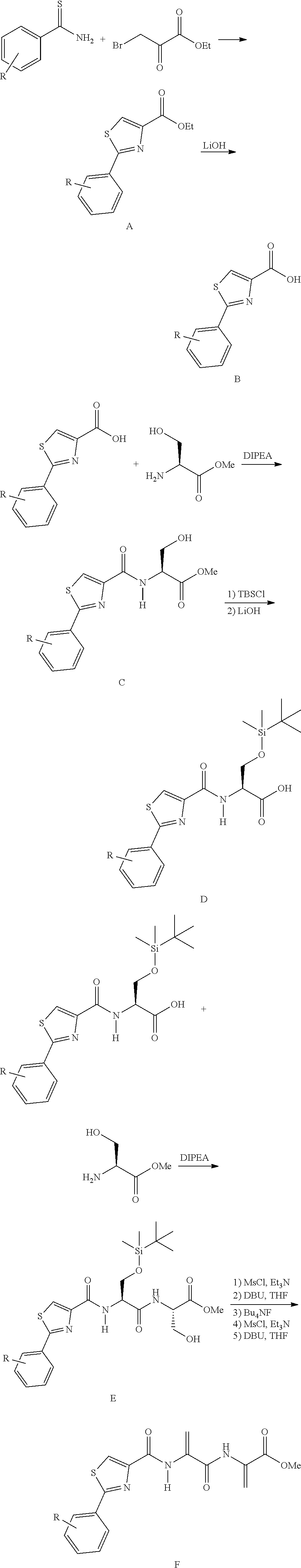

### General Procedures

### Example A

Example A: The thioamide (1.0 eq.) was dissolved in ethanol (0.5M solution). Ethyl bromopyruvate (1.1 eq.) was added and the mixture was heated to 80° C. until the reaction was complete by TLC analysis. The mixture was cooled to RT and concentrated. The residue was suspended in saturated aqueous sodium bicarbonate (50 mL) and the mixture was extracted with ethyl acetate (3×20 mL). The combined organics were dried over magnesium sulfate, filtered, and concentrated. The crude residue was purified by silica gel chromatography to provide the Intermediate A.

### Example B

Example B: Intermediate A (1.0 eq.) was dissolved in 4/1/1 THF/methanol/water. Lithium hydroxide (3.0 eq.) was added and the mixture was stirred at RT until the reaction was complete by TLC analysis. Water (25 mL) was added and the mixture was treated with 1N aq. HCl to pH=4. The resulting mixture was extracted with ethyl acetate (3×20 mL). The combined organics were dried over sodium sulfate, filtered, and concentrated to provide the desired Intermediate B.

### Example C

Example C: Intermediate B (1.0 eq.) was dissolved in DCM. L-Serine methyl ester hydrochloride (1.2 eq.) was added, followed by N,N-diisopropylethylamine (2.0 eq.) and a coupling reagent (1.2 eq.). The mixture was stirred at RT until the reaction was complete by TLC analysis. Water (25 mL) was added and the mixture was extracted with DCM (3×15 mL). The combined organics were dried over sodium sulfate, filtered, and concentrated. The crude residue was purified by silica gel chromatography to provide Intermediate C.

### Example D

Example D: Intermediate C was dissolved in DCM. Imidazole (1.2 eq.) was added followed by tert-butyldimethylchlorosilane (1.2 eq.). The reaction was stirred at RT until complete by TLC analysis. Water ((25 mL) was added and the mixture was extracted with DCM (3×15 mL). The combined organics were dried over sodium sulfate, filtered, and concentrated. The crude residue was purified by silica gel chromatography. The isolated compound was dissolved in 4/1/1 THF/methanol/water. Lithium hydroxide (3.0 eq.) was added and the mixture was stirred at RT until the reaction was complete as judged by TLC. Water (25 mL) was added and the solution was treated with 1N aq. HCl to pH=3. The mixture was extracted with ethyl acetate (3×15 mL). The combined organics were dried over sodium sulfate, filtered, and concentrated to provide Intermediate D.

### Example E

Example E: Intermediate D (1.0 eq.) was dissolved in DCM and L-serine methyl ester hydrochloride (1.2 eq.) was added. N,N-Diisopropylethylamine (2.0 eq.) was added followed by a coupling reagent (1.2 eq.). The mixture was stirred at RT until the reaction was complete by TLC analysis. Water (25 mL) was added and the mixture was extracted with DCM (3×10 mL). The combined organics were dried over sodium sulfate, filtered, and concentrated. The crude residue was purified by silica gel chromatography to provide Intermediate E.

### Example F

Example F: Intermediate E (1.0 eq.) was dissolved in DCM and cooled to 0° C. Triethylamine (1.5 eq.) was added followed by methanesulfonyl chloride (1.5 eq.). The mixture was stirred at 0° until complete by TLC analysis. Water (25 mL) was added and the mixture was extracted with DCM (3×15 mL). The combined organics were dried over sodium sulfate, filtered, and concentrated. The residue was dissolved in THE and cooled to 0° C. 1,8-Diazabicyclo[5.4.0]undec-7-ene (DBU) (1.2 eq.) was added and the mixture was stirred at 0° until the reaction was complete by TLC analysis. Water (25 mL) was added and the mixture was extracted with ethyl acetate (3×15 mL). The combined organics were dried over magnesium sulfate, filtered, and concentrated to provide the desired product. The residue (1.0 eq.) was dissolved in THF. Tetrabutylammonium fluoride (1.1 eq., 1.0 M solution in THF) was added and the solution was stirred at RT until the reaction was complete by TLC analysis. Water (25 mL) was added and the mixture was extracted with ethyl acetate (3×15 mL). The combined organics were dried over magnesium sulfate, filtered, and concentrated. The isolated compound was dissolved in DCM and cooled to 0° C. Triethylamine (1.5 eq.) and methanesulfonyl chloride (1.5 eq.) were added. The mixture was stirred at RT until the reaction was complete by TLC analysis. Water (25 mL) was added and the mixture was extracted with DCM (3×15 mL). The combined organics were dried over sodium sulfate, filtered, and concentrated. The residue was dissolved in THE and cooled to 0° C. 1,8-Diazabicyclo[5.4.0]undec-7-ene (DBU, 1.2 eq.) was added and the mixture was stirred until complete by TLC analysis. Water (25 mL) was added and the mixture was extracted with ethyl acetate (3×15 mL). The combined organics were dried over magnesium sulfate, filtered, and concentrated. The crude residue was purified by silica gel chromatography to provide the desired product.

### Methyl N-(2-bromothiazole-4-carbonyl)-O-(tert-butyldimethylsilyl)-L-serinate

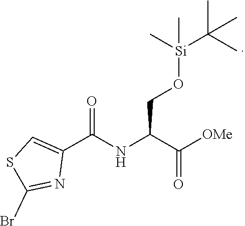 [SMILES: COC(=O)[C@H](CO[Si](C)(C)C(C)(C)C)NC(=O)c1csc(Br)n1]

2-Bromothiazole-4-carboxylic acid (1.807 g, 8.69 mmole) was dissolved in DCM (17 mL). L-Serine methyl ester hydrochloride (1.643 g, 10.6 mmole) was added followed by N,N-diisopropylethylamine (3.00 mL, 17.2 mmole) and pyBOP (5.455 g, 10.5 mmole). The mixture was stirred at RT for 90 minutes. Water (50 mL) was added and the two layers were separated. The aqueous layer was extracted with DCM (2×15 mL). The combined organics were dried over sodium sulfate, filtered, and concentrated. The crude residue was purified by silica gel chromatography (Isco CombiPrep, 24 g RediSep column, 50-90% ethyl acetate/hexane gradient) to provide methyl (2-bromothiazole-4-carbonyl)-L-serinate as a white solid (3.224 g). ¹H NMR (400 MHz, CDCl₃) δ 8.08 (s, 1H), 8.04-7.93 (m, 1H), 4.84 (dt, J=7.7, 3.8 Hz, 1H), 4.11 (dd, J=11.3, 4.0 Hz, 1H), 4.03 (dd, J=11.3, 3.6 Hz, 1H), 3.83 (s, 3H).

Methyl (2-bromothiazole-4-carbonyl)-L-serinate (3.224 g) was dissolved in DCM (20 mL). Imidazole (0.854 g, 12.5 mmole) was added followed by tert-butyldimethylchlorosilane (1.891 g, 12.5 mmole) and the mixture was stirred at RT for 45 minutes. Water (50 mL) was added and the two layers were separated. The aqueous layer was extracted with DCM (2×15 mL). The combined organics were dried over sodium sulfate, filtered, and concentrated. The crude product was purified by silica gel chromatography (Isco CombiPrep, 40 g Silicycle column, 10-40% ethyl acetate/hexane gradient) to provide methyl N-(2-bromothiazole-4-carbonyl)-O-(tert-butyldimethylsilyl)-L-serinate as a pale yellow oil (3.100 g, 84%). ¹H NMR (400 MHz, CDCl₃) δ 8.06 (s, 1H), 7.99-7.87 (m, 1H), 4.80 (dt, J=8.6, 3.1 Hz, 1H), 4.17 (dd, J=10.1, 2.9 Hz, 1H), 3.91 (dd, J=10.1, 3.5 Hz, 1H), 3.78 (s, 3H), 0.90 (s, 9H), 0.06 (s, 3H), 0.05 (s, 3H).

### Compound Synthesis

### Compound 1: Tert-butyl 4-(4-((3-((3-methoxy-3-oxoprop-1-en-2-yl)amino)-3-oxoprop-1-en-2-yl)carbamoyl)thiazol-2-yl)piperidine-1-carboxylate

### Tert-butyl 4-carbamothioylpiperidine-1-carboxylate (1.1)

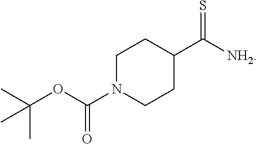 [SMILES: CC(C)(C)OC(=O)N1CCC(C(N)=S)CC1]

1-N-Boc-4-cyanopiperidine (2.005 g, 9.53 mmole) was dissolved in pyridine (10 mL). Triethylamine (1.50 mL, 10.7 mmole) was added followed by ammonium sulfide (40% aqueous solution, 1.80 mL, 10.5 mmole). The mixture was heated to 50° C. for 5 hours, then was cooled to RT and concentrated. The residue was dissolved in ethyl acetate (50 mL). The solution was washed with 1N aq. HCl (2×25 mL). The organics were dried over magnesium sulfate, filtered, and concentrated. The crude residue was purified by silica gel chromatography (Isco CombiPrep, 25 g Silicycle column, 30-60% ethyl acetate/hexane gradient) to provide tert-butyl 4-carbamothioylpiperidine-1-carboxylate as a white solid (1.174 g, 50%). ¹H NMR (400 MHz, Chloroform-d) δ 7.51 (s, 1H), 6.94 (s, 1H), 4.18 (d, J=37.0 Hz, 2H), 2.82-2.62 (m, 3H), 1.96-1.83 (m, 2H), 1.72 (dtd, J=13.2, 12.3, 4.4 Hz, 2H), 1.46 (s, 9H).

### Ethyl 2-(1-(tert-butoxycarbonyl)piperidin-4-yl)thiazole-4-carboxylate (1.2)

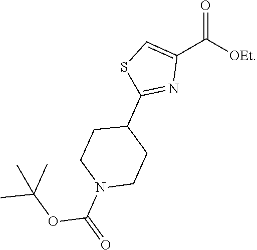 [SMILES: CCOC(=O)c1csc(C2CCN(C(=O)OC(C)(C)C)CC2)n1]

Tert-butyl 4-carbamothioylpiperidine-1-carboxylate (1.174 g, 4.80 mmole) was dissolved in 1,4-dioxane (24 mL) and cooled to 0° C. Potassium bicarbonate (3.868 g, 38.6 mmole) was added followed by ethyl bromopyruvate (1.80 mL, 14.3 mmole). The mixture was stirred at 0° for 4 hours, then was warmed to RT and stirred overnight. The slurry was concentrated to dryness and dissolved in a mixture of water (25 mL) and ethyl acetate (25 mL). The two layers were separated. The organics were washed with sat. aq. sodium chloride (1×25 mL), dried over magnesium sulfate, filtered, and concentrated. The residue was dissolved in 1,4-dioxane (24 mL) and cooled to 0° C. Pyridine (3.10 mL, 38.3 mmole) was added followed by trifluoroacetic anhydride (2.70 mL, 19.4 mmole). The mixture was stirred at 0° for 3 hours, then was warmed to RT and stirred for 2 hours. Triethylamine (16 mL) was added and the mixture was concentrated. The residue was dissolved in ethyl acetate (40 mL) and washed sequentially with 0.5N aq. HCl (2×50 mL), sat. aq. sodium bicarbonate (1×25 mL), and sat. aq. sodium chloride (1×25 mL). The organics were dried over magnesium sulfate, filtered, and concentrated. The crude residue was purified by silica gel chromatography (Isco CombiPrep, 25 g Silicycle column, 15-35% ethyl acetate/hexane gradient) to provide ethyl 2-(1-(tert-butoxycarbonyl)piperidin-4-yl)thiazole-4-carboxylate as a tan solid (1.704 g, quant.). ¹H NMR (400 MHz, Chloroform-d) δ 8.08 (s, 1H), 4.42 (q, J=7.1 Hz, 2H), 4.30-4.16 (m, 2H), 3.26 (tt, J=11.8, 3.7 Hz, 1H), 2.85 (t, J=13.0 Hz, 2H), 2.18-2.07 (m, 2H), 1.80-1.65 (m, 2H), 1.47 (s, 9H), 1.40 (t, J=7.1 Hz, 3H).

### 2-(1-(Tert-butoxycarbonyl)piperidin-4-yl)thiazole-4-carboxylic acid (1.3)

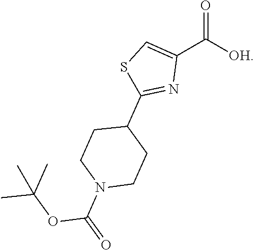 [SMILES: CC(C)(C)OC(=O)N1CCC(c2nc(C(=O)O)cs2)CC1]

Ethyl 2-(1-(tert-butoxycarbonyl)piperidin-4-yl)thiazole-4-carboxylate (1.704 g, 5.01 mmole) was dissolved in 4/1/1 THF/methanol/water (15 mL). Lithium hydroxide (0.484 g, 20.2 mmole) was added and the mixture was stirred at RT for 2 hours, then was concentrated to the aqueous layer. The solid was dissolved in water (200 mL) and the solution was treated with 1N aq. HCl to pH=3. The mixture was extracted with ethyl acetate (5×20 mL) and the combined organics were dried over sodium sulfate, filtered, and concentrated to provide 2-(1-(tert-butoxycarbonyl)piperidin-4-yl)thiazole-4-carboxylic acid as a golden solid (1.451 g, 93%). ¹H NMR (400 MHz, Chloroform-d) δ 8.20 (s, 1H), 4.23 (s, 2H), 3.23 (tt, J=11.7, 3.8 Hz, 1H), 2.88 (t, J=12.7 Hz, 2H), 2.20-2.08 (m, 2H), 1.84-1.67 (m, 2H), 1.48 (s, 9H)

### Tert-butyl (S)-4-(4-((3-hydroxy-1-methoxy-1-oxopropan-2-yl)carbamoyl)thiazol-2-yl)piperidine-1-carboxylate (1.4)

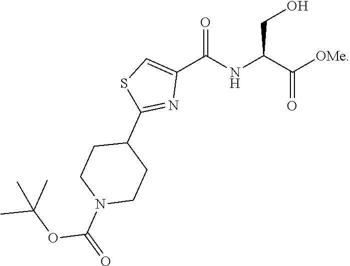 [SMILES: COC(=O)[C@H](CO)NC(=O)c1csc(C2CCN(C(=O)OC(C)(C)C)CC2)n1]

2-(1-(Tert-butoxycarbonyl)piperidin-4-yl)thiazole-4-carboxylic acid (1.451 g, 4.64 mmole) was dissolved in DCM (9 mL). L-Serine methyl ester hydrochloride (0.870 g, 5.59 mmole) was added, followed sequentially by N,N-diisopropylethylamine (2.40 mL, 13.8 mmole) and BOP reagent (2.475 g, 5.60 mmole). The mixture was stirred at RT overnight and water (50 mL) was added. The two layers were separated and the aqueous layer was extracted with DCM (3×20 mL). The combined organics were dried over sodium sulfate, filtered, and concentrated. The crude residue was purified by silica gel chromatography to provide tert-butyl (S)-4-(4-((3-hydroxy-1-methoxy-1-oxopropan-2-yl)carbamoyl)thiazol-2-yl)piperidine-1-carboxylate as a thick, yellow gel (1.763 g, 92%). ¹H NMR (400 MHz, Chloroform-d) δ 8.12 (d, J=7.5 Hz, 1H), 8.02 (s, 1H), 4.84 (dt, J=7.6, 3.8 Hz, 1H), 4.21 (s, 2H), 4.11-4.01 (m, 2H), 3.83 (s, 3H), 3.14 (tt, J=11.6, 3.7 Hz, 1H), 2.89 (t, J=12.4 Hz, 2H), 2.65 (t, J=6.1 Hz, 1H), 2.17-2.07 (m, 2H), 1.75 (qd, J=12.4, 4.3 Hz, 2H), 1.48 (s, 9H).

### Tert-butyl 4-(4-((3-methoxy-3-oxoprop1-en-2-yl)carbamoyl)thiazol-2-yl)piperidine-1-carboxylate (1.5)

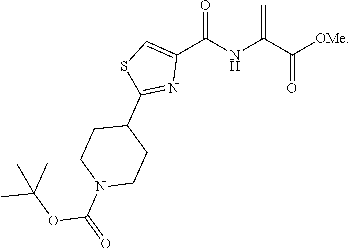 [SMILES: C=C(NC(=O)c1csc(C2CCN(C(=O)OC(C)(C)C)CC2)n1)C(=O)OC]

Tert-butyl (S)-4-(4-((3-hydroxy-1-methoxy-1-oxopropan-2-yl)carbamoyl)thiazol-2-yl)piperidine-1-carboxylate (1.763 g, 4.26 mmole) was dissolved in DCM (9 mL) and cooled to 0° C. Methanesulfonyl chloride (0.365 mL, 4.72 mmole) was added followed by dropwise addition of triethylamine (0.700 mL, 4.99 mmole). The mixture was stirred at 0° for 60 minutes and water (50 mL) was added. The two layers were separated and the aqueous layer was extracted with DCM (2×15 mL). The combined organics were dried over sodium sulfate, filtered, and concentrated. The residue was dissolved in THE (9 mL) and cooled to 0° C. 1,8-Diazabicyclo[5.4.0]undec-7-ene (0.700 mL, 4.68 mmole) was added dropwise and the solution was stirred at 0° for 90 minutes, then at RT overnight. Water (50 mL) was added and the mixture was extracted with ethyl acetate (3×15 mL). The combined organics were dried over magnesium sulfate, filtered, and concentrated. The crude residue was purified by silica gel chromatography to provide tert-butyl 4-(4-((3-methoxy-3-oxoprop1-en-2-yl)carbamoyl)thiazol-2-yl)piperidine-1-carboxylate as a white solid (1.227 g, 73%). ¹H NMR (400 MHz, Chloroform-d) δ9.69 (s, 1H), 8.05 (s, 1H), 6.76 (s, 1H), 5.98 (d, J=1.6 Hz, 1H), 4.32-4.13 (m, 2H), 3.90 (s, 3H), 3.16 (tt, J=11.6, 3.8 Hz, 1H), 2.90 (t, J=12.7 Hz, 2H), 2.21-2.08 (m, 2H), 1.76 (dtd, J =13.1, 11.8, 4.3 Hz, 2H).

### 2-(2-(1-(Tert-butoxycarbonyl)piperidin-4-yl)thiazole-4-carboxamido)acrylic acid (1.6)

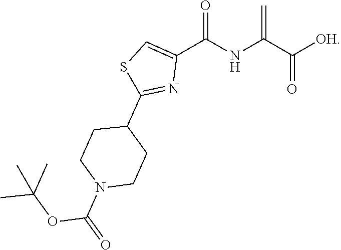 [SMILES: C=C(NC(=O)c1csc(C2CCN(C(=O)OC(C)(C)C)CC2)n1)C(=O)O]

The procedure described in example 1.3 was used to convert tert-butyl 4-(4-((3-methoxy-3-oxoprop1-en-2-yl)carbamoyl)thiazol-2-yl)piperidine-1-carboxylate (1.227 g, 3.10 mmole) to 2-(2-(1-(tert-butoxycarbonyl)piperidin-4-yl)thiazole-4-carboxamido)acrylic acid as a pale yellow solid (1.130 g, 96%). ¹H NMR (400 MHz, DMSO-d6) δ 9.65 (s, 1H), 8.36 (s, 1H), 6.52 (s, 1H), 5.82 (d, J=1.5 Hz, 1H), 4.03 (d, J=13.1 Hz, 2H), 3.38-3.24 (m, 1H), 2.09-2.01 (m, 2H), 1.66-1.50 (m, 2H), 1.41 (s, 9H).

### Tert-butyl (S)-4-(4-((3-((3-hydroxy-1-methoxy-1-oxopropan-2-yl)amino)-3-oxoprop-1-en-2-yl)carbamoyl)thiazol-2-yl)piperidine-1-carboxylate (1.7)

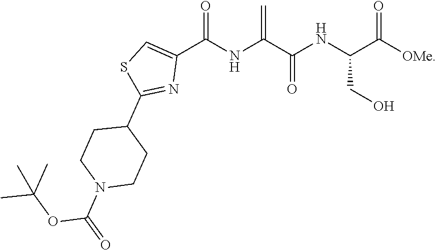 [SMILES: C=C(NC(=O)c1csc(C2CCN(C(=O)OC(C)(C)C)CC2)n1)C(=O)N[C@@H](CO)C(=O)OC]

2-(2-(1-(Tert-butoxycarbonyl)piperidin-4-yl)thiazole-4-carboxamido)acrylic acid (0.706 g, 1.85 mmole) was suspended in DCM (6 mL). L-Serine methyl ester hydrochloride (0.408 g, 2.62 mmole) was added followed by N,N-diisopropylethylamine (0.970 mL, 5.57 mmole). Propylphosphonic acid anhydride (50% solution in 2-MeTHF, 0.650 mL, 2.22 mmole) was added and the mixture was stirred at RT for 90 minutes. The solution was loaded onto a silica gel column and purified by silica gel chromatography (Isco CombiPrep, 25 g Silicycle column, 60-90% ethyl acetate/hexane gradient) to provide tert-butyl (S)-4-(4-((3-((3-hydroxy-1-methoxy-1-oxopropan-2-yl)amino)-3-oxoprop-1-en-2-yl)carbamoyl)thiazol-2-yl)piperidine-1-carboxylate as a white solid (0.310 g, 35%). ¹H NMR (400 MHz, Chloroform-d) δ 9.85 (s, 1H), 8.05 (s, 1H), 7.08 (d, J=7.3 Hz, 1H), 6.64 (d, J=1.9 Hz, 1H), 5.48 (t, J=1.6 Hz, 1H), 4.76 (dt, J=7.0, 3.4 Hz, 1H), 4.20 (s, 2H), 4.12-4.00 (m, 2H), 3.84 (s, 3H), 3.14 (tq, J=11.8, 4.1 Hz, 1H), 2.88 (t, J=12.7 Hz, 2H), 2.49 (s, 1H), 2.21-2.08 (m, 2H), 1.75 (dtd, J=13.3, 11.8, 4.3 Hz, 2H), 1.48 (s, 9H)

### Tert-butyl 4-(4-((3-((3-methoxy-3-oxoprop-1-en-2-yl)amino)-3-oxoprop-1-en-2-yl)carbamoyl)thiazol-2-yl)piperidine-1-carboxylate (1)

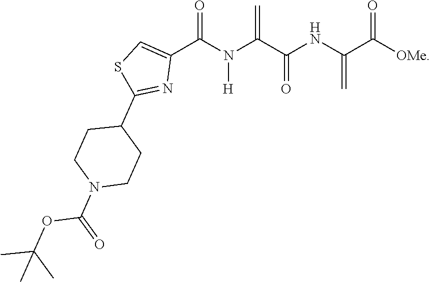 [SMILES: C=C(NC(=O)c1csc(C2CCN(C(=O)OC(C)(C)C)CC2)n1)C(=O)NC(=C)C(=O)OC]

Tert-butyl (S)-4-(4-((3-((3-hydroxy-1-methoxy-1-oxopropan-2-yl)amino)-3-oxoprop-1-en-2-yl)carbamoyl)thiazol-2-yl)piperidine-1-carboxylate (0.310 g, 0.808 mmole) was dissolved in DCM (1.6 mL) and cooled to 0° C. Methanesulfonyl chloride (0.070 mL, 0.904 mmole) was added followed by triethylamine (0.125 mL, 0.892 mmole). The solution was stirred at 0° for 90 minutes and water (25 mL) was added. The mixture was extracted with DCM (3×15 mL). The combined organics were dried over sodium sulfate, filtered, and concentrated. The residue was dissolved in THE (1.6 mL) and cooled to 0° C. 1,8-Diazabicyclo[5.4.0]undec-7-ene (0.145 mL, 0.970 mmole) was added and the mixture was stirred at 0° for 60 minutes. Water (25 mL) was added and the mixture was extracted with ethyl acetate (3×15 mL). The combined organics were dried over magnesium sulfate, filtered, and concentrated. The crude residue was purified by silica gel chromatography (Isco CombiPrep, 12 g RediSep Gold column, 20-40% ethyl acetate/hexane gradient) to provide tert-butyl 4-(4-((3-((3-methoxy-3-oxoprop-1-en-2-yl)amino)-3-oxoprop-1-en-2-yl)carbamoyl)thiazol-2-yl)piperidine-1-carboxylate as a white solid (0.249 g, 66%). ¹H NMR (400 MHz, Chloroform-d) δ 9.89 (s, 1H), 8.54 (s, 1H), 8.06 (s, 1H), 6.75 (d, J=2.2 Hz, 1H), 6.69 (s, 1H), 6.02 (d, J=1.1 Hz, 1H), 5.47 (t, J=1.9 Hz, 1H), 4.22 (s, 2H), 3.90 (s, 3H), 3.17 (tt, J=11.6, 3.7 Hz, 1H), 2.89 (t, J=12.7 Hz, 2H), 2.14 (d, J=13.1 Hz, 2H), 1.84-1.68 (m, 2H), 1.49 (s, 9H).

### Compound 2: Methyl 2-(2-(2-(6-((tert-butoxycarbonyl)amino)pyridine-3-yl)thiazole-4-carboxamido)acrylamido)acrylat

### Tert-butyl 2-(tert-butoxycarbonyl)amino-5-cyanopyridine carbamate (2.1)

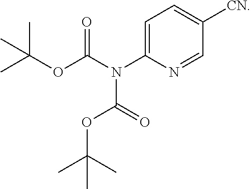 [SMILES: CC(C)(C)OC(=O)N(C(=O)OC(C)(C)C)c1ccc(C#N)cn1]

2-Amino-5-cyanopyridine (3.010 g, 25.3 mmole) was dissolved in DCM (30 mL). Triethylamine (7.00 mL, 49.9 mmole) and 4-dimethylaminopyridine (0.305 g, 0.250 mole) were added. Di-tert-butyl dicarbonate (11.013 g, 50.5 mmole) was added and the solution was stirred at RT for 24 hours. Water (100 mL) was added and the two layers were separated. The aqueous layer was extracted with DCM (2×30 mL). The combined organics were washed with sat. aq. sodium chloride (1×50 mL), then dried over sodium sulfate, filtered, and concentrated. The crude residue was purified by silica gel chromatography (Isco CombiPrep, 40 g Silicycle column, 10-25% ethyl acetate/hexane gradient) to provide tert-butyl 2-(tert-butoxycarbonyl)amino-5-cyanopyridine carbamate as a white solid (7.185 g, 89%). 1H NMR (400 MHz, Chloroform-d) δ 8.66 (dd, J=2.3, 0.8 Hz, 1H), 7.95 (dd, J=8.6, 2.3 Hz, 1H), 7.67 (dd, J=8.6, 0.8 Hz, 1H), 1.51 (s, 18H).

### 2-((Bis-N-Boc)amino)-5-(thiocarboxamido)pyridine (2.2)

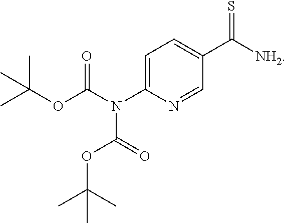 [SMILES: CC(C)(C)OC(=O)N(C(=O)OC(C)(C)C)c1ccc(C(N)=S)cn1]

Tert-butyl 2-(tert-butoxycarbonyl)amino-5-cyanopyridine carbamate (4.994 g, 15.6 mmole) was dissolved in pyridine (16 mL). Triethylamine (2.40 mL, 17.1 mmole) was added followed by ammonium sulfide (40% aqueous solution, 3.20 mL, 18.7 mmole) and the mixture was heated to 50° C. for 7 hours. The mixture was cooled to RT and concentrated. The residue was dissolved in ethyl acetate (50 mL) and washed with water (2×25 mL) and brine (1×25 mL). The organics were dried over magnesium sulfate, filtered, and concentrated. The crude residue was purified by silica gel chromatography (Isco CombiPrep, 40 g Silicycle column, 10-30% ethyl acetate/hexane gradient) to provide 2-((bis-N-Boc)amino)-5-(thiocarboxamido)pyridine as a yellow solid (6.353 g). ¹H NMR (400 MHz, Chloroform-d) δ 8.86 (dd, J=2.6, 0.8 Hz, 1H), 8.28 (dd, J=8.5, 2.6 Hz, 1H), 7.83-7.69 (m, 1H), 7.54 (s, 1H), 7.42 (dd, J=8.5, 0.8 Hz, 1H), 1.49 (s, 18H).

### Ethyl 2-(4-(bis(tert-butoxycarbonyl)amino)pyridine-3-yl)thiazole-4-carboxylate (2.3)

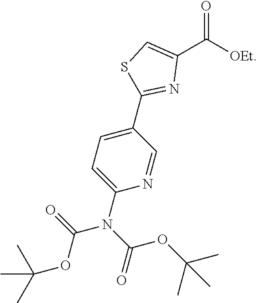 [SMILES: CCOC(=O)c1csc(-c2ccc(N(C(=O)OC(C)(C)C)C(=O)OC(C)(C)C)nc2)n1]

2-((Bis-N-Boc)amino)-5-(thiocarboxamido)pyridine (6.353 g, 18.0 mmole) was dissolved in 1,4-dioxane (90 mL) and cooled to 0° C. Potassium bicarbonate (14.574 g, 146 mmole) was added followed by slow addition of ethyl bromopyruvate (4.50 mL, 35.9 mmole). The mixture was stirred at 0° for 4 hours, then was warmed to RT and allowed to stir overnight. The mixture was concentrated and the residue was suspended in ethyl acetate (100 mL). The mixture was washed sequentially with water (2×50 mL) and sat. aq. sodium chloride (1×50 mL). The organics were dried over magnesium sulfate, filtered, and concentrated. The residue was dissolved in 1,4-dioxane (90 mL) and cooled to 0° C. Pyridine (11.6 mL, 143 mmole) was added followed by slow addition of trifluoroacetic anhydride (5.00 mL, 36.0 mmole). The dark red mixture was stirred at 0° for 3 hours, then at RT for 2 hours. Triethylamine (30 mL) was added and the mixture was concentrated. The residue was suspended in ethyl acetate (75 mL) and washed sequentially with 0.5N aq. HCl (3×50 mL) and sat. aq. Sodium bicarbonate (1×25 mL). The organics were dried over magnesium sulfate, filtered, and concentrated. The crude residue was purified by silica gel chromatography (Isco CombiPrep, 40 g Silicycle column, 10-30% ethyl acetate/hexane gradient) to provide ethyl 2-(4-(bis(tert-butoxycarbonyl)amino)pyridine-3-yl)thiazole-4-carboxylate as a yellow solid (6.302 g, 78%). 1H NMR (400 MHz, Chloroform-d) δ 9.02 (dd, J=2.4, 0.8 Hz, 1H), 8.37 (dd, J=8.4, 2.5 Hz, 1H), 8.22 (s, 1H), 7.44 (dd, J=8.4, 0.8 Hz, 1H), 4.46 (qd, J=7.1, 3.9 Hz, 2H), 1.47 (s, 18H), 1.44 (td, J=7.1, 2.3 Hz, 3H).

### 2-(6-((Tert-butoxycarbonyl)amino)pyridine-3-yl)thiazole-4-carboxylic acid (2.4)

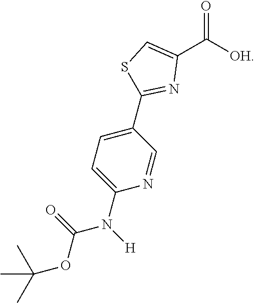 [SMILES: CC(C)(C)OC(=O)Nc1ccc(-c2nc(C(=O)O)cs2)cn1]

Using the procedure described for Example 1.3, ethyl 2-(4-(bis(tert-butoxycarbonyl)amino)pyridine-3-yl)thiazole-4-carboxylate (6.302 g, 14.0 mmole) was converted to 2-(6-((tert-butoxycarbonyl)amino)pyridine-3-yl)thiazole-4-carboxylic acid (3.323 g, 74%) as a white solid. ¹H NMR (400 MHz, DMSO-d6) δ 10.20 (s, 1H), 8.83 (dd, J=2.5, 0.8 Hz, 1H), 8.49 (s, 1H), 8.29 (dd, J=8.8, 2.5 Hz, 1H), 7.97 (dd, J=8.8, 0.8 Hz, 1H), 1.49 (s, 9H).

### Methyl (2-(6-((tert-butyoxycarbonyl)amino)pyridine-3-yl)thiazole-4-carbonyl)-L-serinate (2.5)

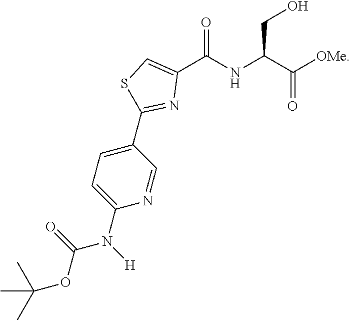 [SMILES: COC(=O)[C@H](CO)NC(=O)c1csc(-c2ccc(NC(=O)OC(C)(C)C)nc2)n1]

Using the procedure described for Example 1.4, 2-(6-((tert-butoxycarbonyl)amino)pyridine-3-yl)thiazole-4-carboxylic acid (3.323 g, 10.3 mmole) was converted to methyl (2-(6-((tert-butyoxycarbonyl)amino)pyridine-3-yl)thiazole-4-carbonyl)-L-serinate (6.065 g) as a white solid. 1H NMR (400 MHz, Chloroform-d) δ 8.79 (dd, J=2.3, 0.8 Hz, 1H), 8.62 (s, 1H), 8.25-8.14 (m, 2H), 8.14-8.05 (m, 2H), 4.88 (dt, J=7.6, 3.7 Hz, 1H), 4.20-4.08 (m, 2H), 3.85 (s, 3H), 1.57 (s, 9H).

### N-(2-(6-((Tert-butoxycarbonyl)amino)pyridine-3-yl)thiazole-4-carbonyl)-O-(tert-butyldimethylsilyl)-L-serine (2.6)

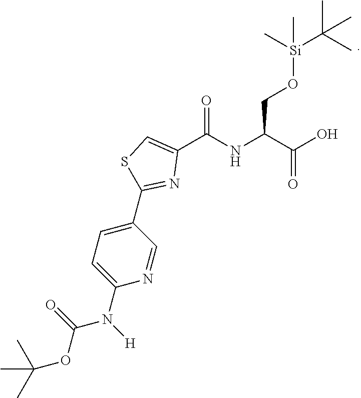 [SMILES: CC(C)(C)OC(=O)Nc1ccc(-c2nc(C(=O)N[C@@H](CO[Si](C)(C)C(C)(C)C)C(=O)O)cs2)cn1]

Methyl 2-(6-((bis(tert-butoxycarbonyl)amino)pyridine-3-yl)thiazole-4-carbonyl)-L-serinate (1.723 g, 3.30 mmole) was dissolved in DMF (6.5 mL). Tert-butyldimethylchlorosilane (0.551 g, 3.66 mmole) was added followed by imidazole (0.274 g, 4.02 mmole) and the solution was stirred at RT for 90 minutes. Water (50 mL) was added and the mixture was extracted with diethyl ether (3×20 mL). The combined organics were dried over magnesium sulfate, filtered, and concentrated. The crude residue was purified by silica gel chromatography to provide methyl O-(tert-butyldimethylsilyl)-N-(6-((bis(tert-butoxycarbonyl)amino)pyridine-3-yl)thiazole-4-carbonyl)-L-serinate as a colorless oil (1.711 g, 81%).

Methyl O-(tert-butyldimethylsilyl)-N-(6-((bis(tert-butoxycarbonyl)amino)pyridine-3-yl)thiazole-4-carbonyl)-L-serinate (1.711 g, 2.69 mmole) was dissolved in 4/1/1 THF/methanol/water (9 mL). Lithium hydroxide (0.322 g, 13.4 mmole) was added and the mixture was stirred at RT for 2 hours, then was concentrated to the aqueous layer. The residue was suspended in water (50 mL) and the mixture was treated with 1N aq. HCl to pH=3. The resulting mixture was extracted with ethyl acetate (4×20 mL). The combined organics were dried over sodium sulfate, filtered, and concentrated to provide N-(2-(6-((tert-butoxycarbonyl)amino)pyridine-3-yl)thiazole-4-carbonyl)-O-(tert-butyldimethylsilyl)-L-serine (4.856 g, 100%) as a white foamy solid. ¹H NMR (400 MHz, DMSO-d6) δ 10.28 (s, 1H), 8.92 (dd, J=2.5, 0.8 Hz, 1H), 8.43 (s, 1H), 8.33 (dd, J=8.8, 2.5 Hz, 1H), 8.22 (d, J=8.6 Hz, 1H), 8.01 (dd, J=8.8, 0.8 Hz, 1H), 4.64 (dt, J=8.6, 3.6 Hz, 1H), 4.15 (dd, J=10.3, 3.6 Hz, 1H), 4.00 (dd, J=10.3, 3.7 Hz, 1H), 1.54 (s, 9H), 0.92 (s, 9H), 0.10 (s, 3H), 0.08 (s, 3H).

### Methyl N-(2-(6-((tert-butoxycarbonyl)amino)pyridine-3-yl)thiazole-4-carbonyl)-O-(tert-butyldimethylsilyl)-L-seryl-L-serinate (2.7)

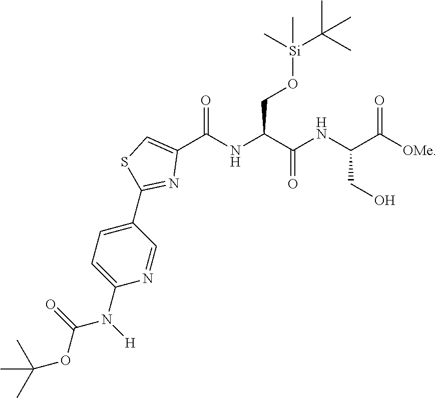 [SMILES: COC(=O)[C@H](CO)NC(=O)[C@H](CO[Si](C)(C)C(C)(C)C)NC(=O)c1csc(-c2ccc(NC(=O)OC(C)(C)C)nc2)n1]

Using the general procedure described for Example 1.4, N-(2-(6-((tert-butoxycarbonyl)amino)pyridine-3-yl)thiazole-4-carbonyl)-O-(tert-butyldimethylsilyl)-L-serine (3.010 g, 5.76 mmole) was converted to methyl N-(2-(6-((tert-butoxycarbonyl)amino)pyridine-3-yl)thiazole-4-carbonyl)-O-(tert-butyldimethylsilyl)-L-seryl-L-serinate (3.276 g, 91%) as a white solid. ¹H NMR (400 MHz, Chloroform-d) δ 8.82-8.75 (m, 1H), 8.25-8.16 (m, 2H), 8.12 (d, J=5.6 Hz, 1H), 8.06 (dt, J=8.8, 1.1 Hz, 1H), 7.85 (d, J=19.8 Hz, 1H), 7.46 (d, J=7.3 Hz, 1H), 4.74-4.61 (m, 2H), 4.20 (dd, J=9.8, 4.1 Hz, 1H), 4.06-3.93 (m, 2H), 3.86 (ddd, J=9.8, 8.3, 6.3 Hz, 1H), 3.78 (s, 3H), 1.56 (s, 9H), 0.94 (s, 9H), 0.14 (s, 3H), 0.13 (s, 3H).

### Methyl 2-(2-(2-(6-((tert-butoxycarbonyl)amino)pyridine-3-yl)thiazole-4-carboxamido)acrylamido acrylate (2)

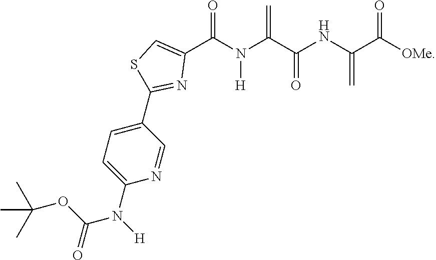 [SMILES: C=C(NC(=O)c1csc(-c2ccc(NC(=O)OC(C)(C)C)nc2)n1)C(=O)NC(=C)C(=O)OC]

Methyl N-(2-(6-((tert-butoxycarbonyl)amino)pyridine-3-yl)thiazole-4-carbonyl)-O-(tert-butyldimethylsilyl)-L-seryl-L-serinate (1.148 g, 1.84 mmole) was dissolved in DCM (4 mL) and cooled to 0° C. Methanesulfonyl chloride (0.160 mL, 2.07 mmole) was added followed by triethylamine (0.290 mL, 2.07 mmole). The mixture was stirred at 0° for 90 minutes and water (50 mL) was added. The mixture was extracted with DCM (3×15 mL). The combined organics were dried over sodium sulfate, filtered, and concentrated. The residue was dissolved in THE (4 mL) and cooled to 0° C. 1,8-Diazabicyclo[5.4.0]undec-7-end (0.300 mL, 2.01 mmole) was added and the solution was stirred at 0° for 2 hours, then was warmed to RT and stirred overnight. Water (50 mL) was added and the mixture was extracted with ethyl acetate (3×15 mL). The combined organics were dried over magnesium sulfate, filtered, and concentrated. The crude residue was purified by silica gel chromatography to provide methyl (S)-2-(2-(2-(6-((tert-butoxycarbonyl)amino)pyridin-3-yl)thiazole-4-carboxamido)-3-((tert-butyldimethylsilyl)oxy)propanamido)acrylate as a white solid (0.838 g, 75%).

Methyl (S)-2-(2-(2-(6-((tert-butoxycarbonyl)amino)pyridin-3-yl)thiazole-4-carboxamido)-3-((tert-butyldimethylsilyl)oxy)propanamido)acrylate (0.838 g, 1.38 mmole) was dissolved in THE (2.8 mL) and cooled to 0° C. Tetrabutylammonium fluoride (1.0.M solution in THF, 1.55 mL, 1.55 mmole) was added and the solution was stirred at 0° for 4 hours. Water (50 mL) was added and the two layers were extracted with ethyl acetate (3×15 mL). The combined organics were dried over magnesium sulfate, filtered, and concentrated. The crude residue was purified by silica gel chromatography to provide the desired product as a white solid (0.558 g, 82%).

Using the dehydration procedure described above, methyl (S)-2-(2-(2-(6-((tert-butoxycarbonyl)amino)pyridine-3-yl)thiazole-4-carboxamido)-3-hydroxypropanamido)acrylate (0.558 g, 1.14 mmole) was converted to methyl 2-(2-(2-(6-((tert-butoxycarbonyl)amino)pyridine-3-yl)thiazole-4-carboxamido)acrylamide)acrylate (0.162 g, 19%) as a white solid._¹H NMR (400 MHz, Chloroform-d) δ 10.03-9.93 (m, 1H), 8.91 (dd, J=2.4, 0.8 Hz, 1H), 8.55 (s, 1H), 8.32 (s, 1H), 8.25 (ddd, J=8.8, 2.4, 0.5 Hz, 1H), 8.15 (s, 1H), 8.10 (dd, J=8.8, 0.8 Hz, 1H), 6.78 (d, J=2.3 Hz, 1H), 6.71 (s, 1H), 6.03 (d, J=1.3 Hz, 1H), 5.51 (t, J=1.9 Hz, 1H), 3.91 (s, 3H), 1.56 (s, 9H).

### Compound 3: Tert-butyl 3-(4-((3-((3-methoxy-3-oxoprop-1-en-2-yl)amino)-3-oxoprop-1-en-2-yl)carbamoyl)thiazol-2-yl)piperidine-1-carboxylate

### Tert-butyl 3-(4-(((S)-3-((tert-butyldimethylsilyl)oxy)-1-methoxy-1-oxopropan-2-yl)carbamoyl)thiazol-2-yl)piperidine-1-carboxylate (3.1)

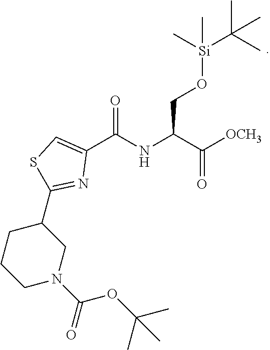 [SMILES: COC(=O)[C@H](CO[Si](C)(C)C(C)(C)C)NC(=O)c1csc(C2CCCN(C(=O)OC(C)(C)C)C2)n1]

Using the procedure described for Example 1, 1—N-Boc-3-cyanopiperidine (2.505 g, 11.9 mmole) was converted to tert-butyl 3-(4-(((S)-3-((tert-butyldimethylsilyl)oxy)-1-methoxy-1-oxopropan-2-yl)carbamoyl)thiazol-2-yl)piperidine-1-carboxylate (1.002 g). ¹H NMR (400 MHz, Chloroform-d) δ 8.08 (d, J=8.7 Hz, 1H), 8.01 (s, 1H), 4.88-4.78 (m, 1H), 4.30 (q, J=7.1 Hz, 1H), 4.23-4.14 (m, 1H), 4.01 (d, J=13.3 Hz, 1H), 3.92 (dd, J=10.1, 3.5 Hz, 1H), 3.78 (s, 3H), 3.14 (dt, J=10.6, 6.1 Hz, 2H), 2.97-2.82 (m, 1H), 2.27-2.15 (m, 1H), 1.90-1.73 (m, 2H), 1.63 (s, 1H), 1.48 (s, 9H), 0.89 (s, 9H), 0.06 (s, 3H), 0.04 (s, 3H).

### N-(2-(1-(Tert-butoxycarbonyl)piperidine-3-yl)thiazole-4-carbonyl)-O-(tert-butyldimethylsilyl)-L-serine (3.2)

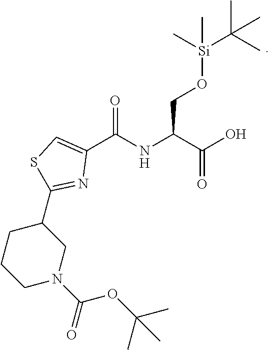 [SMILES: CC(C)(C)OC(=O)N1CCCC(c2nc(C(=O)N[C@@H](CO[Si](C)(C)C(C)(C)C)C(=O)O)cs2)C1]

Using the procedure described for Example 1.3, tert-butyl 3-(4-(((S)-3-((tert-butyldimethylsilyl)oxy)-1-methoxy-1-oxopropan-2-yl)carbamoyl)thiazol-2-yl)piperidine-1-carboxylate (1.002 g, 1.90 mmole) was converted to N-(2-(1-(tert-butoxycarbonyl)piperidine-3-yl)thiazole-4-carbonyl)-O-(tert-butyldimethylsilyl)-L-serine (1.041 g) as a colorless oil. ¹H NMR (400 MHz, Chloroform-d) δ 8.13 (d, J=8.2 Hz, 1H), 8.06 (d, J=7.4 Hz, 1H), 4.90-4.79 (m, 1H), 4.41-4.27 (m, 1H), 4.24 (dd, J=10.1, 3.0 Hz, 1H), 4.08-3.98 (m, 1H), 3.95 (dd, J=10.1, 4.0 Hz, 1H), 3.21-3.00 (m, 2H), 2.98-2.83 (m, 1H), 2.28-2.14 (m, 1H), 1.89-1.72 (m, 2H), 1.60 (d, J=20.6 Hz, 1H), 1.47 (s, 10H), 0.90 (s, 9H), 0.08 (d, J=2.3 Hz, 6H)

### Tert-butyl 3-(4-(((4S,7S)-4-(hydroxymethyl)-10,10,11,11-tetramethyl-3,6-dioxo-2,9-dioxa-5-aza-10-siladodecan-7-yl)carbamoyl)thiazol-2-yl)piperidine-1-carboxylate (3.3)

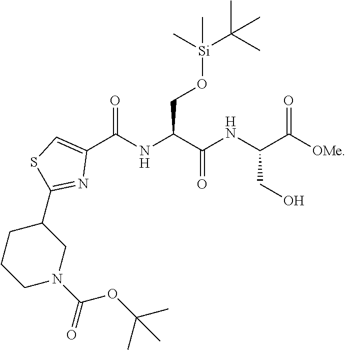 [SMILES: COC(=O)[C@H](CO)NC(=O)[C@H](CO[Si](C)(C)C(C)(C)C)NC(=O)c1csc(C2CCCN(C(=O)OC(C)(C)C)C2)n1]

Using the general procedure described for Example 1.4, N-(2-(1-(tert-butoxycarbonyl)piperidine-3-yl)thiazole-4-carbonyl)-O-(tert-butyldimethylsilyl)-L-serine (1.041 g, 2.03 mmole) was converted to tert-butyl 3-(4-(((4S,7S)-4-(hydroxymethyl)-10,10,1 1,11-tetramethyl-3,6-dioxo-2,9-dioxa-5-aza-10-siladodecan-7-yl)carbamoyl)thiazol-2-yl)piperidine-1-carboxylate (0.728 g, 58%) as a colorless oil. ¹H NMR (400 MHz, Chloroform-d) δ 8.12 (d, J=7.0 Hz, 1H), 8.06-7.98 (m, 1H), 7.44 (d, J=7.2 Hz, 1H), 4.68 (dt, J=7.3, 3.7 Hz, 1H), 4.66-4.57 (m, 1H), 4.21-4.15 (m, 1H), 3.99 (t, J=7.7 Hz, 3H), 3.84 (dtd, J=9.8, 6.9, 1.2 Hz, 1H), 3.78 (d, J=4.6 Hz, 3H), 3.14 (s, 2H), 2.99-2.83 (m, 2H), 2.28-2.13 (m, 1H), 1.90-1.73 (m, 3H), 1.60 (t, J=11.8 Hz, 1H), 1.47 (d, J=3.2 Hz, 9H), 0.92 (s, 9H), 0.13 (s, 3H), 0.12 (s, 3H)

### Tert-butyl 3-(4-((3-((3-methoxy-3-oxoprop-1-en-2-yl)amino)-3-oxoprop-1-en-2-yl)carbamoyl)thiazol-2-yl)piperidine-1-carboxylate (3)

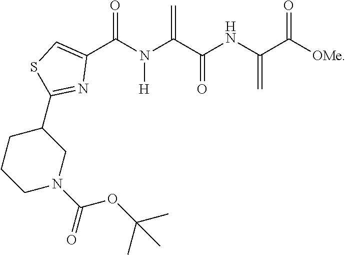 [SMILES: C=C(NC(=O)c1csc(C2CCCN(C(=O)OC(C)(C)C)C2)n1)C(=O)NC(=C)C(=O)OC]

Using the procedure described for Compound 2, tert-butyl 3-(4-(((4S,7S)-4-(hydroxymethyl)-10,10,11,11-tetramethyl-3,6-dioxo-2,9-dioxa-5-aza-10-siladodecan-7-yl)carbamoyl)thiazol-2-yl)piperidine-1-carboxylate (0.728 g, 1.18 mmole) was converted to tert-butyl 3-(4-((3-((3-methoxy-3-oxoprop-1-en-2-yl)amino)-3-oxoprop-1-en-2-yl)carbamoyl)thiazol-2-yl)piperidine-1-carboxylate (0.126 g, 23%) as a white solid. ¹H NMR (400 MHz, Chloroform-d) δ 9.89 (s, 1H), 8.53 (s, 1H), 8.06 (s, 1H), 6.74 (d, J=2.2 Hz, 1H), 6.69 (s, 1H), 6.01 (d, J=1.2 Hz, 1H), 5.47 (t, J=1.9 Hz, 1H), 4.39-4.26 (m, 1H), 4.09-3.94 (m, 1H), 3.90 (s, 3H), 3.25-3.03 (m, 2H), 3.01-2.86 (m, 1H), 2.31-2.16 (m, 1H), 1.90-1.73 (m, 2H), 1.64 (s, 1H), 1.48 (s, 9H).

### Compound 4: Methyl 2-(2-(2-(4-(6-bromohexanamido)phenyl)thiazole-4-carboxamido)acrylamido)acrylate

### 6-Bromo-N-(4-(4,4,5,5-tetramethyl-1,3,2-dioxaborolan-2-yl)phenyl)hexanamide (4.1)

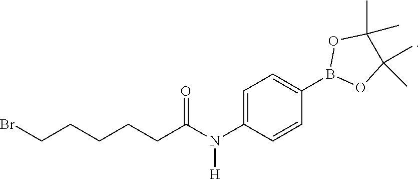 [SMILES: CC1(C)OB(c2ccc(NC(=O)CCCCCBr)cc2)OC1(C)C]

6-Bromohexanoic acid (1.964 g, 10.1 mmole) was dissolved in DCM (20 mL) and DMF (1 drop) was added. Oxalyl chloride (0.880 mL, 10.1 mmole) was added dropwise and the solution was stirred at RT for 90 minutes, then was concentrated. The residue was dissolved in DCM (2 mL) and added dropwise to cold (0° C.) solution of 4-(4,4,5,5-tetramethyl-1,3,2-dioxaborolan-2-yl)aniline (2.007 g, 9.16 mmole) and N,N-diisopropylethylamine (3.20 mL, 18.4 mmole) in DCM (20 mL). The resulting solution was stirred at 0° for 60 minutes then at RT for 2 hours. Water (50 mL) was added and the two layers were separated. The mixture was extracted with DCM (2×20 mL). The combined organics were dried over sodium sulfate, filtered, and concentrated. The crude residue was purified by silica gel chromatography (Isco CombiPrep, 24 g RediSep column, 10-30% ethyl acetate/hexane gradient) to provide 6-bromo-N-(4-(4,4,5,5-tetramethyl-1,3,2-dioxaborolan-2-yl)phenyl)hexanamide as an orange solid (3.241 g, 89%). 1H NMR (400 MHz, Chloroform-d) δ 7.81-7.72 (m, 2H), 7.52 (d, J=8.1 Hz, 2H), 7.17 (s, 1H), 3.42 (t, J=6.7 Hz, 2H), 2.38 (t, J=7.4 Hz, 2H), 1.96-1.85 (m, 2H), 1.79 (d, J=7.5 Hz, 2H), 1.57-1.48 (m, 2H), 1.34 (s, 12H).

### Methyl N-(2-(4-(6-bromohexanamido)phenyl)thiazole-4-carbonyl)-O-(tert-butyldimethylsilyl)-L-serinate (4.2)

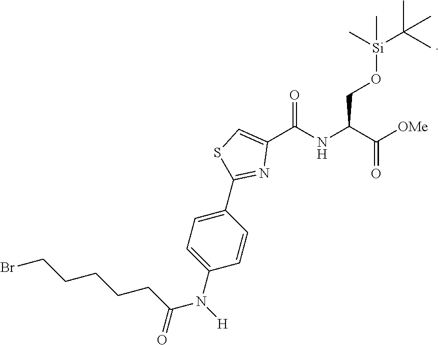 [SMILES: COC(=O)[C@H](CO[Si](C)(C)C(C)(C)C)NC(=O)c1csc(-c2ccc(NC(=O)CCCCCBr)cc2)n1]

Methyl N-(2-bromothiazole-4-carbonyl)-O-(tert-butyldimethylsilyl)-L-serinate (2.006 g, 4.74 mmole) was dissolved in 1,4-dioxane (9 mL). 6-Bromo-N-(4,4,5,5-tetramethyl-1,3,2-dioxaborolan-2-yl)phenyl)hexanamide (1.880 g, 4.75 mmole) was added followed by potassium carbonate (2M aqueous solution, 4.70 mL, 9.40 mmole) and bis(triphenylphosphine)palladium(II) chloride (0.332 g, 0.473 mmole). The mixture was heated to 85° C. for 18 hours and cooled to RT. Water (50 mL) was added and the mixture was extracted with ethyl acetate (3×20 mL). The combined organics were dried over magnesium sulfate, filtered, and concentrated. The crude residue was purified by silica gel chromatography (Isco CombiPrep, 24 g RediSep column, 20-50% ethyl acetate/hexane gradient) to provide methyl N-(2-(4-(6-bromohexanamido)phenyl)thiazole-4-carbonyl)-O-(tert-butyldimethylsilyl)-L-serinate as a thick, orange gel (1.290 g, 44%). ¹H NMR (400 MHz, Chloroform-d) δ 8.22 (d, J=8.7 Hz, 1H), 8.05 (s, 1H), 7.94-7.85 (m, 2H), 7.67-7.60 (m, 2H), 7.51 (s, 1H), 4.90-4.81 (m, 1H), 4.22 (dd, J=10.1, 2.6 Hz, 1H), 3.95 (dd, J=10.0, 3.4 Hz, 1H), 3.79 (s, 3H), 3.43 (t, J=6.7 Hz, 2H), 2.41 (t, J=7.4 Hz, 2H), 1.92 (dq, J=8.1, 6.8 Hz, 2H), 1.84-1.72 (m, 2H), 1.59-1.50 (m, 2H), 0.92 (s, 9H), 0.08 (s, 3H), 0.06 (s, 3H).

### N-(2-(4-(6-Bromohexanamido)phenyl)thiazole)-4-carbonyl)-O-(tert-butyldimethylsilyl)-L-serine (4.3)

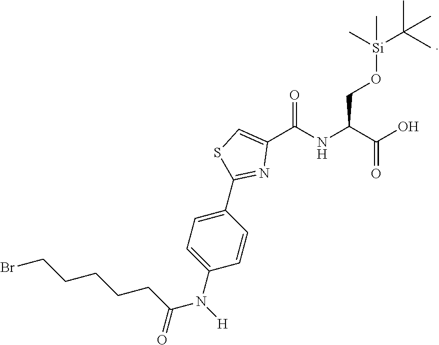 [SMILES: CC(C)(C)[Si](C)(C)OC[C@H](NC(=O)c1csc(-c2ccc(NC(=O)CCCCCBr)cc2)n1)C(=O)O]

Methyl N-(2-(4-(6-bromohexanamido)phenyl)thiazole-4-carbonyl)-O-(tert-butyldimethylsilyl)-L-serinate (1.290 g, 2.11 mmole) was dissolved in 4/1/1 THF/methanol/water (12 mL). Lithium hydroxide (0.158 g, 6.60 mmole) was added and the mixture was stirred at RT for 2 hours. The mixture was poured into water (40 mL) and the solution was treated with 1N aq. HCl to pH=4. The slurry was extracted with ethyl acetate (3×15 mL). The combined organics were dried over sodium sulfate, filtered, and concentrated to provide N-(2-(4-(6-bromohexanamido)phenyl)thiazole)-4-carbonyl)-O-(tert-butyldimethylsilyl)-L-serine as an orange foam (1.213 g, 96%). ¹H NMR (400 MHz, Chloroform-d) δ 8.26 (d, J=8.7 Hz, 1H), 8.05 (s, 1H), 7.84-7.81 (m, 2H), 7.74 (s, 1H), 7.67-7.60 (m, 2H), 4.90-4.81 (m, 1H), 4.26 (dd, J=10.1, 2.6 Hz, 1H), 3.95 (dd, J=10.0, 3.4 Hz, 1H), 3.43 (t, J=6.7 Hz, 2H), 2.41 (t, J=7.4 Hz, 2H), 1.92 (dq, J=8.1, 6.8 Hz, 2H), 1.84-1.72 (m, 2H), 1.55-1.48 (m, 2H), 0.92 (s, 9H), 0.08 (s, 3H), 0.06 (s, 3H).

### Methyl N-(2-(4-(6-bromohexanamido)phenyl)thiazole-4-carbonyl)-O-(tert-butyldimethylsilyl)-L-seryl-L-serinate (4.4)

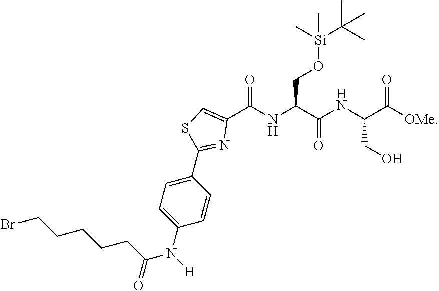 [SMILES: COC(=O)[C@H](CO)NC(=O)[C@H](CO[Si](C)(C)C(C)(C)C)NC(=O)c1csc(-c2ccc(NC(=O)CCCCCBr)cc2)n1]

Using the procedure described for Example 1.4, N-(2-(4-(6-bromohexanamido)phenyl)thiazole)-4-carbonyl)-O-(tert-butyldimethylsilyl)-L-serine (1.213 g, 2.03 mmole) was converted to methyl N-(2-(4-(6-bromohexanamido)phenyl)thiazole-4-carbonyl)-O-(tert-butyldimethylsilyl)-L-seryl-L-serinate (1.152 g, 81%) as a yellow gel. ¹H NMR (400 MHz, Chloroform-d) δ 8.24 (d, J=7.0 Hz, 1H), 8.06 (s, 1H), 7.90-7.84 (m, 2H), 7.65-7.60 (m, 2H), 7.49-7.45 (m, 1H), 7.42 (s, 1H), 4.69 (dq, J=7.6, 3.8 Hz, 1H), 4.64 (dt, J =6.7, 3.3 Hz, 1H), 4.25-4.16 (m, 1H), 4.05-3.93 (m, 2H), 3.89-3.81 (m, 1H), 3.79 (s, 3H), 3.44 (t, J=6.7 Hz, 2H), 2.43 (t, J=7.4 Hz, 2H), 1.84-1.73 (m, 3H), 1.57 (q, J=8.3 Hz, 3H), 0.95 (s, 9H), 0.14 (s, 3H), 0.14 (s, 3H).

### Methyl 2-(2-(2-(4-(6-bromohexanamido)phenyl)thiazole-4-carboxamido)acrylamido)acrylate (4)

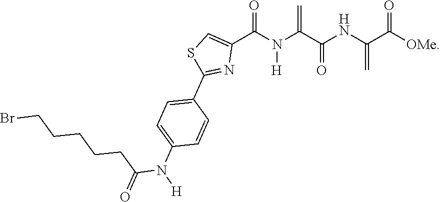 [SMILES: C=C(NC(=O)c1csc(-c2ccc(NC(=O)CCCCCBr)cc2)n1)C(=O)NC(=C)C(=O)OC]

Using the procedure described for Compound 2, methyl N-(2-(4-(6-bromohexanamido)phenyl)thiazole-4-carbonyl)-O-(tert-butyldimethylsilyl)-L-seryl-L-serinate (1.151 g, 1.64 mmole) was converted to methyl 2-(2-(2-(4-(6-bromohexanamido)phenyl)thiazole-4-carboxamido)acrylamide)acrylate (0.275 g, 31%) as a white solid. ¹H NMR (400 MHz, Chloroform-d) δ 10.00 (s, 1H), 8.55 (s, 1H), 8.11 (s, 1H), 8.04-7.90 (m, 2H), 7.64 (d, J=8.3 Hz, 2H), 6.78 (d, J=2.2 Hz, 1H), 6.74-6.65 (m, 1H), 6.03 (d, J=1.2 Hz, 1H), 5.50 (t, J=1.9 Hz, 1H), 3.91 (s, 3H), 3.44 (t, J=6.7 Hz, 2H), 2.43 (t, J=7.4 Hz, 2H), 1.93 (dq, J=9.1, 6.8 Hz, 2H), 1.80 (p, J=7.6 Hz, 2H), 1.63-1.50 (m, 2H).

### Compound 5: Methyl 2-(2-(2-(4-((tert-butoxycarbonyl)amino)phenyl)thiazole-4-carboxamido)acrylamido)acrylate

### Ethyl 2-(4-((tert-butoxycarbonyl)amino)phenyl)thiazole-4-carboxylate (5.1)

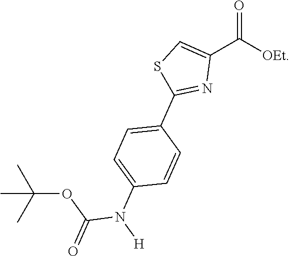 [SMILES: CCOC(=O)c1csc(-c2ccc(NC(=O)OC(C)(C)C)cc2)n1]

Ethyl 2-bromothiazole-4-carboxylate (1.006 g, 4.26 mmole) was dissolved in 1,4-dioxane (8.5 mL). Tert-butyl (4-(4,4,5,5-tetramethyl-1,3,2-dioxaborolan-2-yl)phenyl)carbamate (1.491 g, 4.67 mmole) was added followed by potassium carbonate (2M aqueous solution, 4.20 mL, 8.40 mmole) and bis(triphenylphosphine)palladium(II) chloride (0.149 g, 0.212 mmole). The mixture was heated to 90° C. for 20 hours and was cooled to RT. Water (50 mL) was added and the mixture was extracted with ethyl acetate (3×15 mL). The combined organics were dried over magnesium sulfate, filtered, and concentrated. The crude residue was purified by silica gel chromatography (Isco CombiPrep, 24 g RediSep column, 10-25% ethyl acetate/hexane gradient) to provide ethyl 2-(4-((tert-butoxycarbonyl)amino)phenyl)thiazole-4-carboxylate as a white solid (0.557 g, 38%). ¹H NMR (400 MHz, Chloroform-d) δ 8.10 (s, 1H), 7.99-7.90 (m, 2H), 7.50-7.41 (m, 2H), 6.62 (s, 1H), 4.44 (q, J=7.1 Hz, 2H), 1.53 (s, 9H), 1.43 (t, J=7.1 Hz, 3H).

### 2-(4-((Tert-butoxycarbonyl)amino)phenyl)thiazole-4-carboxylic acid (5.2)

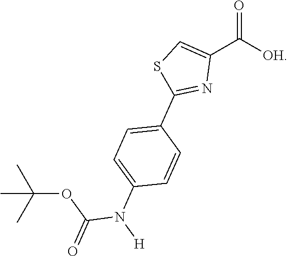 [SMILES: CC(C)(C)OC(=O)Nc1ccc(-c2nc(C(=O)O)cs2)cc1]

Using the procedure described for Example 1.3, ethyl 2-(4-((tert-butoxycarbonyl)amino)phenyl)thiazole-4-carboxylate (0.557 g, 1.60 mmole) was converted to 2-(4-((tert-butoxycarbonyl)amino)phenyl)thiazole-4-carboxylic acid (0.442 g, 86%) as a white solid. ¹H NMR (400 MHz, DMSO-d₆) δ 13.06 (s, 1H), 9.69 (s, 1H), 8.41 (s, 1H), 7.94-7.78 (m, 2H), 7.67-7.54 (m, 2H), 1.50 (s, 9H).

### 2,5-Dioxopyrrolidin-1-yl 2-(4-((tert-butoxycarbonyl)amino)phenyl)thiazole-4-carboxylate (5.3)

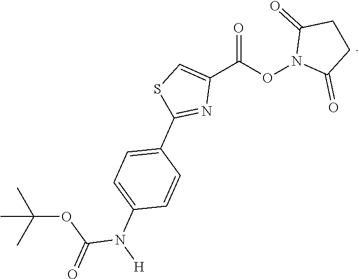 [SMILES: CC(C)(C)OC(=O)Nc1ccc(-c2nc(C(=O)ON3C(=O)CCC3=O)cs2)cc1]

2-(4-((Tert-butoxycarbonyl)amino)phenyl)thiazole-4-carboxylic acid (0.442 g, 1.38 mmole) was dissolved in DMF (5 mL). N-Hydroxysuccinimide (0.194 g, 1.69 mmole) was added followed by EDC·HCl (0.533 g, 2.78 mmole) and the mixture was stirred at RT for 90 minutes. Water (50 mL) was added and the mixture was extracted with ethyl acetate (3×15 mL). The combined organics were dried over magnesium sulfate, filtered, and concentrated. The crude residue was purified by silica gel chromatography (Isco CombiPrep, 12 g RediSep column, 30-50% ethyl acetate/hexane gradient) to provide 2,5-dioxopyrrolidin-1-yl 2-(4-((tert-butoxycarbonyl)amino)phenyl)thiazole-4-carboxylate as a white solid (0.376 g, 65%). ¹H NMR (400 MHz, Chloroform-d) δ 8.38 (s, 1H), 7.97-7.87 (m, 2H), 7.53-7.42 (m, 2H), 6.69 (s, 1H), 2.92 (s, 4H), 1.53 (s, 9H).

### Methyl (2-(4-((tert-butoxycarbonyl)amino)phenyl)-thiazole-4-carbonyl)-L-serinate (5.4)

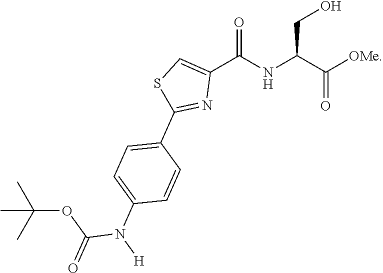 [SMILES: COC(=O)[C@H](CO)NC(=O)c1csc(-c2ccc(NC(=O)OC(C)(C)C)cc2)n1]

2,5-Dioxopyrrolidin-1-yl 2-(4-((tert-butoxycarbonyl)amino)phenyl)thiazole-4-carboxylate (0.376 g, 0.901 mmole) was suspended in DCM (4 mL). L-Serine methyl ester hydrochloride (0.168 g, 1.08 mmole) was added followed by N,N-diisopropylethylamine (0.320 mL, 1.84 mmole). The mixture was stirred at RT for 20 hours and water (25 mL) was added. The mixture was extracted with DCM (3×10 mL). The combined organics were dried over sodium sulfate, filtered, and concentrated. The crude residue was purified by silica gel chromatography (Isco CombiPrep, 12 g RediSep column, 40-70% ethyl acetate/hexane gradient) to provide methyl (2-(4-((tert-butoxycarbonyl)amino)phenyl)-thiazole-4-carbonyl)-L-serinate as a white solid (0.355 g, 93%). ¹H NMR (400 MHz, Chloroform-d) δ 8.24 (d, J=7.5 Hz, 1H), 8.06 (s, 1H), 7.94-7.85 (m, 2H), 7.51-7.43 (m, 2H), 6.64 (s, 1H), 4.88 (dt, J=7.6, 3.8 Hz, 1H), 4.17-4.05 (m, 2H), 3.85 (s, 3H), 2.63 (t, J=6.2 Hz, 1H), 1.54 (s, 9H).

### N-(2-(4-((Tert-butoxycarbonyl)amino)phenyl)thiazole-4-carbonyl)-O-(tert-butyldimethylsilyl)-L-serine (5.5)

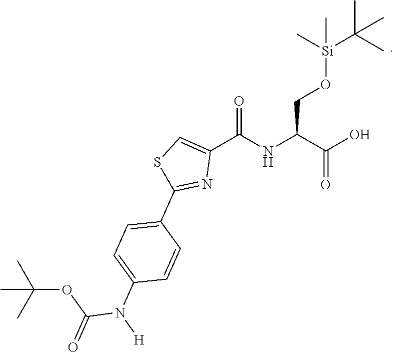 [SMILES: CC(C)(C)OC(=O)Nc1ccc(-c2nc(C(=O)N[C@@H](CO[Si](C)(C)C(C)(C)C)C(=O)O)cs2)cc1]

Using the general procedure described for Example 4.2 and 4.3, methyl (2-(4-((tert-butoxycarbonyl)amino)phenyl)-thiazole-4-carbonyl)-L-serinate (0.355 g, 0.842 mmole) was converted to N-(2-(4-((tert-butoxycarbonyl)amino)phenyl)thiazole-4-carbonyl)-O-(tert-butyldimethylsilyl)-L-serine (0.297 g, 68%) as a white solid. ¹H NMR (400 MHz, Chloroform-d) δ 8.25 (d, J=7.7 Hz, 1H), 8.08 (s, 1H), 7.94-7.86 (m, 2H), 7.45 (d, J=8.4 Hz, 2H), 4.84 (dt, J =8.0, 4.1 Hz, 1H), 4.27 (dd, J=10.1, 3.4 Hz, 1H), 3.98 (dd, J=10.0, 4.4 Hz, 1H), 1.56 (s, 9H), 0.92 (s, 9H), 0.08 (s, 6H).

### Methyl N-(2-(4-((tert-butoxycarbonyl)amino)phenyl)thiazole-4-carbonyl)-O-(tert-butyldimethylsilyl)-L-seryl-L-serinate (5.6)

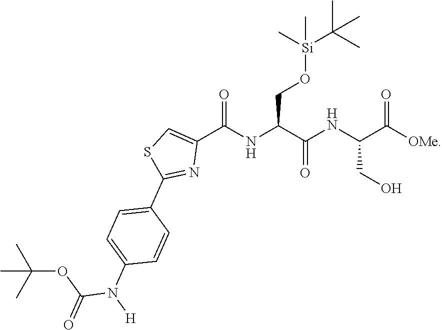 [SMILES: COC(=O)[C@H](CO)NC(=O)[C@H](CO[Si](C)(C)C(C)(C)C)NC(=O)c1csc(-c2ccc(NC(=O)OC(C)(C)C)cc2)n1]

Using the procedure described for Example 1.4, N-(2-(4-((tert-butoxycarbonyl)amino)phenyl)thiazole-4-carbonyl)-O-(tert-butyldimethylsilyl)-L-serine (0.297 g, 0.569 mmole) was converted to methyl N-(2-(4-((tert-butoxycarbonyl)amino)phenyl)thiazole-4-carbonyl)-O-(tert-butyldimethylsilyl)-L-seryl-L-serinate (0.385 g) as a colorless solid using pyBOP as the coupling reagent. ¹H NMR (400 MHz, Chloroform-d) δ 8.25 (d, J=7.0 Hz, 1H), 8.06 (d, J=4.9 Hz, 1H), 7.91-7.82 (m, 2H), 7.50-7.39 (m, 3H), 6.66 (s, 1H), 4.69 (dt, J=7.1, 3.6 Hz, 1H), 4.64 (dq, J=10.6, 4.0, 3.3 Hz, 1H), 4.21 (ddd, J=9.8, 3.9, 1.0 Hz, 1H), 3.99 (dd, J=3.7, 1.0 Hz, 2H), 3.84 (dd, J=9.8, 6.6 Hz, 1H), 3.78 (s, 3H), 1.54 (s, 9H), 0.94 (s, 9H), 0.14 (s, 3H), 0.13 (s, 3H).

### Methyl 2-(2-(2-(4-((tert-butoxycarbonyl)amino)phenyl)thiazole-4-carboxamido)acrylamido)acrylate (5)

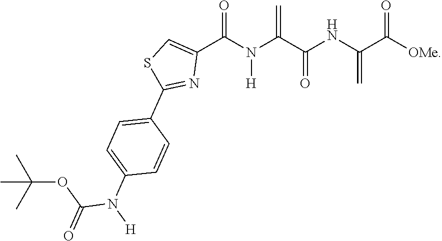 [SMILES: C=C(NC(=O)c1csc(-c2ccc(NC(=O)OC(C)(C)C)cc2)n1)C(=O)NC(=C)C(=O)OC]

Using the procedure describing Compound 5, methyl N-(2-(4-((tert-butoxycarbonyl)amino)phenyl)thiazole-4-carbonyl)-O-(tert-butyldimethylsilyl)-L-seryl-L-serinate (0.385 g, 0.618 mmole) was converted to methyl 2-(2-(2-(4-((tert-butoxycarbonyl)amino)phenyl)thiazole-4-carboxamido)acrylamido)acrylate (0.033 g, 12%). ¹H NMR (400 MHz, DMSO-d₆) δ 9.97 (s, 1H), 9.93 (s, 1H), 9.72 (s, 1H), 8.40 (s, 1H), 7.96-7.84 (m, 2H), 7.69-7.58 (m, 2H), 6.51 (d, J=1.3 Hz, 1H), 5.87 (s, 2H), 5.84 (s, 1H), 3.75 (s, 3H), 1.49 (s, 9H).

### Compound 6: Methyl 2-(2-(2-(6-((6-((methylsulfonyl)oxy)hexyl)carbamoyl)pyridine-3-yl)thiazole-4-carboxamido)acrylamido)acrylate

### 5-Bromo-N-(6-hydroxyhexyl)picolinamide (6.1)

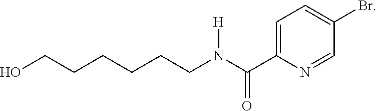 [SMILES: O=C(NCCCCCCO)c1ccc(Br)cn1]

5-Bromopicolinic acid (1.006 g, 4.98 mmole) was suspended in DCM (10 mL) and N,N′-carbonyldiimidazole (0.887 g, 5.47 mmole) was added (gas evolution was observed). The mixture was stirred at RT for 90 minutes and 6-aminohexan-1-ol (0.706 g, 6.02 mmole) was added. The resulting solution was stirred at RT for 3 hours then water (50 mL) was added. The two layers were separated and the aqueous layer was extracted with DCM (2×15 mL). The combined organics were dried over sodium sulfate, filtered, and concentrated. The crude residue was purified by silica gel chromatography to provide 5-bromo-N-(6-hydroxyhexyl)picolinamide (1.019 g, 68%) as a white solid. ¹H NMR (400 MHz, Chloroform-d) δ 8.60 (dd, J=2.2, 0.8 Hz, 1H), 8.09 (dd, J=8.3, 0.7 Hz, 1H), 7.98 (dd, J=8.4, 2.3 Hz, 1H), 7.93 (s, 1H), 3.64 (t, J=6.5 Hz, 2H), 3.47 (td, J=7.1, 6.2 Hz, 2H), 1.76-1.55 (m, 4H), 1.43 (p, J=3.6 Hz, 4H).

### 5-Bromo-N-(6-((tert-butyldimethylsilyl)oxy)hexyl)picolinamide (6.2)

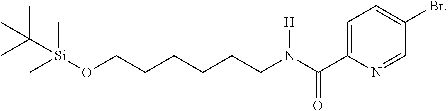 [SMILES: CC(C)(C)[Si](C)(C)OCCCCCCNC(=O)c1ccc(Br)cn1]

5-Bromo-N-(6-hydroxyhexyl)picolinamide (1.019 g, 3.38 mmole) was dissolved in DMF (7 mL). Imidazole (0.258 g, 3.79 mmole) was added followed by tert-butyldimethylchlorosilane (0.571 g, 3.79 mmole). The solution was stirred at RT for 2 hours and was poured into water (50 mL). The mixture was extracted with ethyl acetate (3×15 mL). The combined organics were dried over magnesium sulfate, filtered, and concentrated. The crude residue was purified by silica gel chromatography to provide 5-bromo-N-(6-((tert-butyldimethylsilyl)oxy)hexyl)picolinamide as a colorless oil (1.260 g, 90%). ¹H NMR (400 MHz, Chloroform-d) δ 8.59 (dd, J=2.3, 0.8 Hz, 1H), 8.09 (dd, J=8.3, 0.7 Hz, 1H), 7.97 (dd, J=8.3, 2.3 Hz, 1H), 7.91 (s, 1H), 3.60 (t, J=6.5 Hz, 2H), 3.45 (td, J=7.2, 6.1 Hz, 2H), 1.64 (dd, J=8.5, 6.0 Hz, 2H), 1.53 (p, J=6.6 Hz, 2H), 1.45-1.34 (m, 4H), 0.89 (s, 9H), 0.04 (s, 6H).

### N-(6-((Tert-butyldimethylsilyl)oxy)hexyl)-5-(4,4,5,5-tetramethyl-1,3,2-dioxaborolan-2-yl)picolinamide (6.3)

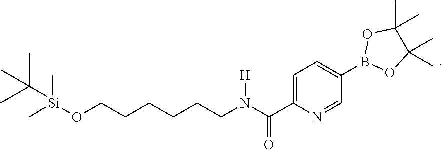 [SMILES: CC1(C)OB(c2ccc(C(=O)NCCCCCCO[Si](C)(C)C(C)(C)C)nc2)OC1(C)C]

5-Bromo-N-(6-((tert-butyldimethylsilyl)oxy)hexyl)picolinamide (1.260 g, 3.03 mmole) was dissolved in 1,4-dioxane (18 mL) and placed under an argon atmosphere Bis(pinacolato)diboron (1.165 g, 4.59 mmole) was added followed by potassium acetate (0.745 g, 7.59 mmole) and 1,1-bis(diphenylphosphino)ferrocenepalladium(II) dichloride (0.221 g, 0.302 mmole). The mixture was heated to 85° C. for 18 hours and was cooled to RT. Water (75 mL) was added and the mixture was extracted with ethyl acetate (3×25 mL). The combined organics were dried over magnesium sulfate, filtered, and concentrated. The crude residue was purified by silica gel chromatography to provide N-(6-((tert-butyldimethylsilyl)oxy)hexyl)-5-(4,4,5,5-tetramethyl-1,3,2-dioxaborolan-2-yl)picolinamide as a brown oil (1.952 g). ¹H NMR (400 MHz, Chloroform-d) δ 8.85 (dd, J=1.6, 1.0 Hz, 1H), 8.21 (dd, J=7.7, 1.7 Hz, 1H), 8.17 (dd, J=7.8, 1.0 Hz, 1H), 8.12 (d, J=6.2 Hz, 1H), 3.60 (t, J=6.5 Hz, 2H), 3.47 (td, J=7.2, 6.1 Hz, 2H), 1.65 (h, J=6.8 Hz, 2H), 1.53 (p, J=6.6 Hz, 2H), 1.36 (s, 16H), 0.89 (s, 9H), 0.04 (s, 6H).

### Methyl O-(tert-butyldimethylsilyl)-N-(2-(6-((5-(tert-butyldimethylsilyl)oxy)pentyl)carbamoyl)pyridine-3-yl)thiazole-4-carbonyl)-L-serinate (6.4)

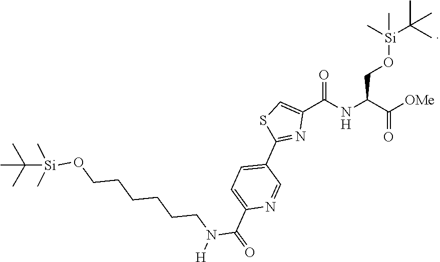 [SMILES: COC(=O)[C@H](CO[Si](C)(C)C(C)(C)C)NC(=O)c1csc(-c2ccc(C(=O)NCCCCCCO[Si](C)(C)C(C)(C)C)nc2)n1]

Methyl N-(2-bromothiazole-4-carbonyl)-O-(tert-butyldimethylsilyl)-L-serinate (0.759 g, 1.79 mmole) was dissolved in 1,4-dioxane (3.6 mL) and N-(6-((tert-butyldimethylsilyl)oxy)hexyl)-5-(4,4,5,5-tetramethyl-1,3,2-dioxaborolan-2-yl)picolinamide (1.073 g, 2.32 mmole) was added. Potassium carbonate (2M aqueous solution, 1.80 mL, 3.60 mmole) and bis(triphenylphosphine)palladium(II) chloride (0.132 g, 0.188 mmole) were added and the mixture was heated to 85° C. for 3 hours. The mixture was cooled to RT and poured into water (50 mL) and was extracted with ethyl acetate (3×15 mL). The combined organics were dried over magnesium sulfate, filtered, and concentrated. The crude residue was purified by silica gel chromatography to provide methyl O-(tert-butyldimethylsilyl)-N-(2-(6-((5-(tert-butyldimethylsilyl)oxy)pentyl)carbamoyl)pyridine-3-yl)thiazole-4-carbonyl)-L-serinate as an orange oil (0.733 g, 62%). ¹H NMR (400 MHz, Chloroform-d) δ 9.10 (dd, J=2.2, 0.9 Hz, 1H), 8.40 (dd, J=8.2, 2.2 Hz, 1H), 8.30 (dd, J=8.2, 0.9 Hz, 1H), 8.23 (s, 1H), 8.19 (d, J=8.8 Hz, 1H), 8.04 (t, J=6.0 Hz, 1H), 4.87 (dt, J=8.7, 3.0 Hz, 1H), 4.22 (dd, J=10.1, 2.6 Hz, 1H), 3.96 (dd, J=10.1, 3.4 Hz, 1H), 3.80 (s, 3H), 3.61 (t, J=6.5 Hz, 2H), 3.50 (td, J=7.2, 6.1 Hz, 2H), 1.68 (p, J=7.4 Hz, 2H), 1.54 (q, J=6.7 Hz, 2H), 1.42 (dt, J=8.7, 5.5 Hz, 4H), 0.92 (s, 9H), 0.89 (s, 9H), 0.08 (s, 3H), 0.06 (s, 3H), 0.05 (s, 6H).

### O-(Tert-butyldimethylsilyl)-N-(2-(6-((5-((tert-butyldimethylsilyl)oxy)pentyl)carbamoyl)pyridine-3-yl)thiazole-4-carbonyl)-L-serine (6.5)

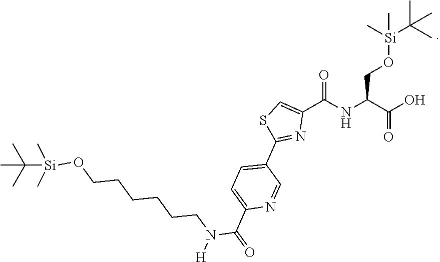 [SMILES: CC(C)(C)[Si](C)(C)OCCCCCCNC(=O)c1ccc(-c2nc(C(=O)N[C@@H](CO[Si](C)(C)C(C)(C)C)C(=O)O)cs2)cn1]

Methyl O-(tert-butyldimethylsilyl)-N-(2-(6-((5-(tert-butyldimethylsilyl)oxy)pentyl)carbamoyl)pyridine-3-yl)thiazole-4-carbonyl)-L-serinate (0.733 g, 1.10 mmole) was dissolved in 4/1/1 THF/methanol/water (6 mL). Lithium hydroxide (0.080 g, 3.34 mmole) was added and the mixture was stirred at RT for 3 hours. The mixture was poured into water (25 mL) and treated with 1N aq. HCl to pH=4. The slurry was extracted with ethyl acetate (3×10 mL). The combined organics were dried over sodium sulfate, filtered, and concentrated to provide O-(tert-butyldimethylsilyl)-N-(2-(6-((5-((tert-butyldimethylsilyl)oxy)pentyl)carbamoyl)pyridine-3-yl)thiazole-4-carbonyl)-L-serine as an orange solid (0.677 g, 95%). 1H NMR (400 MHz, Chloroform-d) δ 9.08 (dd, J=2.2, 0.8 Hz, 1H), 8.47-8.40 (m, 1H), 8.36 (dt, J=8.2, 1.1 Hz, 1H), 8.28 (d, J=8.8 Hz, 1H), 8.23 (d, J=8.1 Hz, 1H), 8.09 (t, J=6.1 Hz, 1H), 4.88 (ddd, J=8.1, 4.1, 3.0 Hz, 1H), 4.33-4.24 (m, 1H), 3.99 (dd, J=10.2, 4.0 Hz, 1H), 3.62 (t, J=6.4 Hz, 2H), 3.51 (q, J=7.0 Hz, 2H), 1.67 (q, J=7.6, 7.2 Hz, 2H), 1.59-1.50 (m, 2H), 1.42 (tq, J=9.5, 5.3, 4.2 Hz, 4H), 0.94-0.91 (m, 9H), 0.89 (s, 9H), 0.09 (s, 6H), 0.05 (s, 6H).

### Methyl O-(tert-butyldimethylsilyl)-N-(2-(6-((5-((tert-butyldimethylsilyl)oxy)pentyl)carbamoyl)pyridine-3-yl)thiazole-4-carbonyl)-L-seryl-L-serinate (6.6)

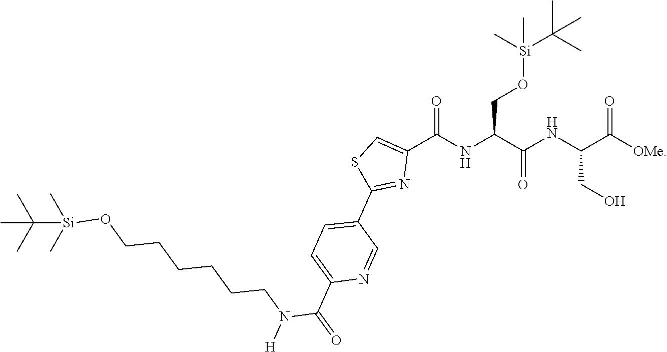 [SMILES: COC(=O)[C@H](CO)NC(=O)[C@H](CO[Si](C)(C)C(C)(C)C)NC(=O)c1csc(-c2ccc(C(=O)NCCCCCCO[Si](C)(C)C(C)(C)C)nc2)n1]

Using the procedure described for Example 1.4, 0-(tert-butyldimethylsilyl)-N-(2-(6-((5-((tert-butyldimethylsilyl)oxy)pentyl)carbamoyl)pyridine-3-yl)thiazole-4-carbonyl)-L-serine (0.677 g, 1.04 mmole) was converted to methyl O-(tert-butyldimethylsilyl)-N-(2-(6-((5-((tert-butyldimethylsilyl)oxy)pentyl)carbamoyl)pyridine-3-yl)thiazole-4-carbonyl)-L-seryl-L-serinate (0.534 g, 68%) as a pale yellow oil using pyBOP as the coupling reagent. 1H NMR (400 MHz, Chloroform-d) δ 9.09 (ddd, J=4.3, 2.2, 0.9 Hz, 1H), 8.39 (ddd, J=8.2, 4.8, 2.2 Hz, 1H), 8.33-8.26 (m, 1H), 8.24 (d, J=3.8 Hz, 2H), 8.04 (t, J=6.0 Hz, 1H), 7.47 (d, J=7.1 Hz, 1H), 4.75-4.61 (m, 2H), 4.22 (dt, J=9.8, 3.8 Hz, 1H), 4.04-3.96 (m, 2H), 3.86 (dt, J=9.9, 6.5 Hz, 1H), 3.79 (s, 3H), 3.61 (t, J=6.5 Hz, 2H), 3.50 (q, J=6.8 Hz, 2H), 1.69 (q, J=7.2 Hz, 2H), 1.54 (p, J=6.7 Hz, 2H), 1.41 (dd, J=7.4, 4.3 Hz, 4H), 0.95 (s, 9H), 0.89 (s, 9H), 0.16 (s, 3H), 0.14 (s, 3H), 0.05 (s, 6H).

### Methyl 2-(2-(2-(6-((6-((methylsulfonyl)oxy)hexyl)carbamoyl)pyridine-3-yl)thiazole-4-carboxamido)acrylamido)acrylate (6)

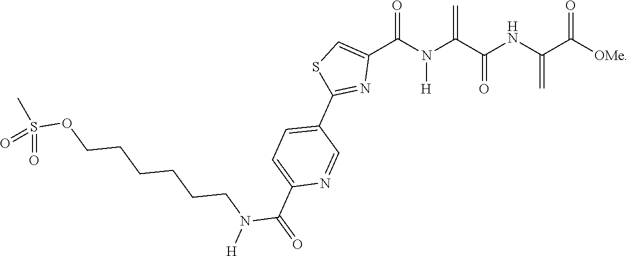 [SMILES: C=C(NC(=O)c1csc(-c2ccc(C(=O)NCCCCCCOS(C)(=O)=O)nc2)n1)C(=O)NC(=C)C(=O)OC]

Using the procedure described for Compound 2, methyl O-(tert-butyldimethylsilyl)-N-(2-(6-((5-((tert-butyldimethylsilyl)oxy)pentyl)carbamoyl)pyridine-3-yl)thiazole-4-carbonyl)-L-seryl-L-serinate (0.534 g, 0.710 mmole) was converted to methyl 2-(2-(2-(6-((6-((methylsulfonyl)oxy)hexyl)carbamoyl)pyridine-3-yl)thiazole-4-carboxamido)acrylamide)acrylate (0.073 g, 18%) as a white solid. ¹H NMR (400 MHz, Chloroform-d) δ 10.02 (s, 1H), 9.19 (dd, J=2.2, 0.8 Hz, 1H), 8.57 (s, 1H), 8.44 (dd, J=8.1, 2.2 Hz, 1H), 8.31 (dd, J=8.1, 0.8 Hz, 1H), 8.27 (s, 1H), 8.06 (t, J=6.1 Hz, 1H), 6.80 (d, J=2.3 Hz, 1H), 6.75-6.66 (m, 1H), 6.09-5.98 (m, 1H), 5.53 (t, J=2.0 Hz, 1H), 4.24 (t, J=6.5 Hz, 2H), 3.91 (s, 3H), 3.52 (q, J=6.8 Hz, 2H), 3.01 (s, 3H), 1.79 (t, J=7.0 Hz, 2H), 1.69 (p, J=7.1 Hz, 2H), 1.52-1.43 (m, 4H).

### Compound 7: Methyl 2-(2-(2-(4-methoxyphenyl)thiazole-4-carboxamido)acrylamido)acrylate

### Ethyl 2-(4-methoxyphenyl)thiazole-4-carboxylate (7.1)

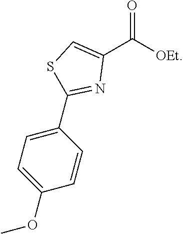 [SMILES: CCOC(=O)c1csc(-c2ccc(OC)cc2)n1]

Ethyl 2-bromothiazole-4-carboxylate (0.500 g, 2.12 mmole) was dissolved in 1,4-dioxane (4 mL). 2-(4-Methoxyphenyl)-4,4,5,5-tetramethyl-1,3,2-dioxaborolane (0.593 g, 2.53 mmole) was added followed by potassium carbonate (2M aqueous solution, 2.10 mL, 4.20 mmole) and bis(triphenylphosphine)palladium(II) chloride (0.147 g, 0.209 mmole). The mixture was heated to 80° C. for 5 hours and was cooled to RT. Water (25 mL) was added and the mixture was extracted with ethyl acetate (3×10 mL). The combined organics were dried over magnesium sulfate, filtered, and concentrated. The crude residue was purified by silica gel chromatography (Isco CombiPrep, 12 g Silicycle column, 10-30% ethyl acetate/hexane gradient) to provide ethyl 2-(4-methoxyphenyl)thiazole-4-carboxylate as a white solid (0.249 g, 45%). ¹H NMR (400 MHz, Chloroform-d) δ 8.09 (s, 1H), 7.99-7.90 (m, 2H), 7.00-6.90 (m, 2H), 4.44 (q, J=7.1 Hz, 2H), 3.87 (s, 3H), 1.43 (t, J=7.1 Hz, 3H).

### 2-(4-Methoxyphenyl)thiazole-4-carboxylic acid (7.2)

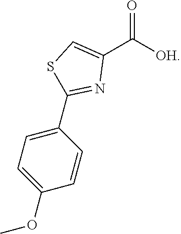 [SMILES: COc1ccc(-c2nc(C(=O)O)cs2)cc1]

Using the procedure described for Example 4.3, ethyl 2-(4-methoxyphenyl)thiazole-4-carboxylate (0.249 g, 0.946 mmole) was converted to 2-(4-methoxyphenyl)thiazole-4-carboxylic acid (0.248 g, 100%) as a cream colored solid. ¹H NMR (400 MHz, DMSO-d₆) δ 13.05 (s, 1H), 8.41 (s, 1H), 7.98-7.85 (m, 2H), 7.15-7.01 (m, 2H), 3.84 (s, 3H).

### Methyl (2-(4-methoxyphenyl)thiazole-4-carbonyl)-L-serinate (7.3)

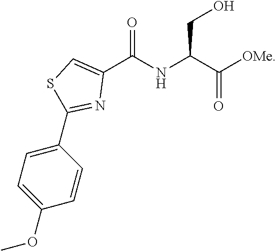 [SMILES: COC(=O)[C@H](CO)NC(=O)c1csc(-c2ccc(OC)cc2)n1]

Using the procedure described for Example 1.4, 2-(4-methoxyphenyl)thiazole-4-carboxylic acid (0.248 g, 1.05 mmole) was converted to methyl (2-(4-methoxyphenyl)thiazole-4-carbonyl)-L-serinate (0.292 g, 83%) as a white solid. ¹H NMR (400 MHz, Chloroform-d) δ 8.26 (d, J=7.5 Hz, 1H), 8.05 (s, 1H), 7.96-7.86 (m, 2H), 7.02-6.92 (m, 2H), 4.88 (dt, J=7.6, 3.8 Hz, 1H), 4.17-4.05 (m, 2H), 3.88 (s, 3H), 3.85 (s, 3H).

### O-(Tert-butyldimethylsilyl)-N-(2-(4-methoxyphenyl)thiazole-4-carbonyl)-L-serine (7.4)

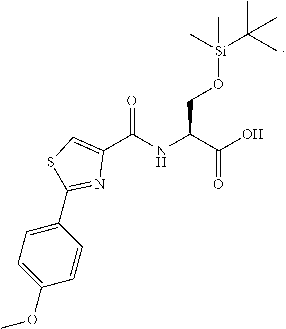 [SMILES: COc1ccc(-c2nc(C(=O)N[C@@H](CO[Si](C)(C)C(C)(C)C)C(=O)O)cs2)cc1]

Using the procedure described for Example 4.2 and 4.3, methyl (2-(4-methoxyphenyl)thiazole-4-carbonyl)-L-serinate (0.292 g, 0.868 mmole) was converted to 0-(tert-butyldimethylsilyl)-N-(2-(4-methoxyphenyl)thiazole-4-carbonyl)-L-serine (0.269 g) as a mixture of products that was carried on without additional purification.

### Methyl O-(tert-butydimethylsilyl)-N-(2-(4-methoxyphenyl)thiazole-4-carbonyl)-L-seryl-L-serinate (7.5)

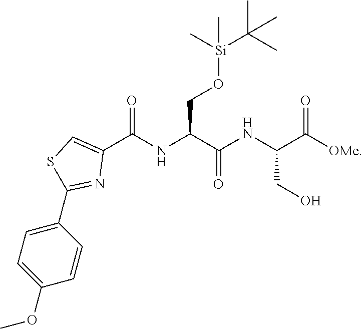 [SMILES: COC(=O)[C@H](CO)NC(=O)[C@H](CO[Si](C)(C)C(C)(C)C)NC(=O)c1csc(-c2ccc(OC)cc2)n1]

Using the procedure described for Example 1.4, O-(tert-butyldimethylsilyl)-N-(2-(4-methoxyphenyl)thiazole-4-carbonyl)-L-serine (0.269 g) was converted to methyl O-(tert-butydimethylsilyl)-N-(2-(4-methoxyphenyl)thiazole-4-carbonyl)-L-seryl-L-serinate (0.097 g, 29%) as a white solid. ¹H NMR (400 MHz, CDCl₃) δ 8.26 (d, J=7.0 Hz, 1H), 8.03 (s, 1H), 7.93-7.83 (m, 2H), 7.46 (d, J=7.1 Hz, 1H), 6.99-6.90 (m, 2H), 4.70 (dd, J=7.2, 3.7 Hz, 1H), 4.65 (td, J=6.9, 4.2 Hz, 1H), 4.21 (dd, J=9.8, 4.1 Hz, 1H), 3.99 (dd, J=3.7, 1.0 Hz, 2H), 3.88 (s, 3H), 3.84 (dd, J=9.8, 6.7 Hz, 1H), 3.79 (s, 3H), 0.95 (s, 9H), 0.14 (s, 3H), 0.13 (s, 3H).

### Methyl 2-(2-(2-(4-methoxyphenyl)thiazole-4-carboxamido)acrylamido)acrylate (7)

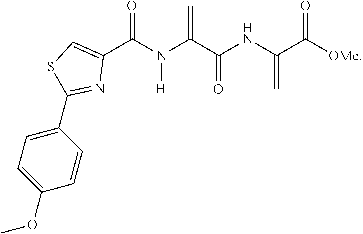 [SMILES: C=C(NC(=O)c1csc(-c2ccc(OC)cc2)n1)C(=O)NC(=C)C(=O)OC]

Using the procedure described in Compound 2, methyl O-(tert-butydimethylsilyl)-N-(2-(4-methoxyphenyl)thiazole-4-carbonyl)-L-seryl-L-serinate (0.097 g, 0.180 mmole) was converted to methyl 2-(2-(2-(4-methoxyphenyl)thiazole-4-carboxamido)acrylamido)acrylate (0.0203 g, 29%) as a white solid. ¹H NMR (400 MHz, CDCl₃) δ 10.02 (s, 1H), 8.55 (s, 1H), 8.07 (s, 1H), 8.00-7.92 (m, 2H), 7.02-6.92 (m, 2H), 6.78 (d, J=2.2 Hz, 1H), 6.71 (s, 1H), 6.03 (d, J=1.3 Hz, 1H), 5.49 (t, J=1.9 Hz, 1H), 3.90 (s, 3H), 3.88 (s, 3H).

### Compound 8: Methyl 2-(2-(2-(4-(((tert-butoxycarbonyl)amino)methyl)phenyl)thiazole-4-carboxamido)acrylamido)acrylate

### Tert-butyl (4-bromobenzyl)carbamate (8.1)

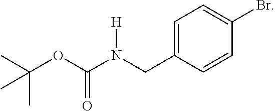 [SMILES: CC(C)(C)OC(=O)NCc1ccc(Br)cc1]

4-Bromobenzylamine (2.006 g, 10.8 mmole) was dissolved in DCM (22 mL) and N,N-diisopropylethylamine (2.45 mL, 14.1 mmole) was added. Di-tert-butyl dicarbonate (2.600 g, 11.9 mmole) was added and the solution was stirred at RT for 5 hours. The mixture was concentrated and the crude residue was purified by silica gel chromatography (Isco CombiPrep, 40 g Silicycle column, 10-25% ethyl acetate/hexane gradient) to provide tert-butyl (4-bromobenzyl)carbamate as a white solid (2.732 g, 88%). ¹H NMR (400 MHz, Chloroform-d) δ 7.48-7.39 (m, 2H), 7.20-7.11 (m, 2H), 4.84 (s, 1H), 4.26 (d, J=6.1 Hz, 2H), 1.46 (s, 9H)

### Tert-butyl (4-(4,4,5,5-tetramethyl-1,3,2-dioxaborolan-2-yl)benzyl)carbamate (8.2)

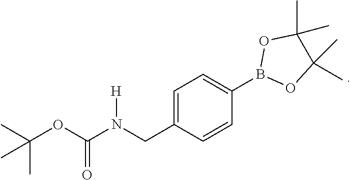 [SMILES: CC(C)(C)OC(=O)NCc1ccc(B2OC(C)(C)C(C)(C)O2)cc1]

Tert-butyl (4-bromobenzyl)carbamate (2.732 g, 9.55 mmole) was dissolved in 1,4-dioxane (19 mL). Bis(pinacolato)diboron (2.914 g, 11.5 mmole) was added followed by potassium acetate (2.032 g, 20.7 mmole) and 1,1′-bis(diphenylphosphino)ferrocenepalladium(II) dichloride (0.697 g, 0.953 mmole). The mixture was heated to 80° C. for 5 hours and was cooled to RT. Water (50 mL) was added and the mixture was extracted with ethyl acetate (3×20 mL). The combined organics were dried over magnesium sulfate, filtered, and concentrated. The crude residue was purified by silica gel chromatography (Isco CombiPrep, 40 g Silicycle column, 5-20% ethyl acetate/hexane gradient) to provide tert-butyl (4-(4,4,5,5-tetramethyl-1,3,2-dioxaborolan-2-yl)benzyl)carbamate as a golden oil (4.231 g) that was carried on without additional purification. ¹H NMR (400 MHz, Chloroform-d) δ 7.80-7.73 (m, 2H), 7.28 (d, J=8.1 Hz, 2H), 4.89 (s, 1H), 4.32 (d, J=6.0 Hz, 2H), 1.45 (s, 9H), 1.34 (s, 12H).

### Ethyl 2-(4-(((tert-butoxycarbonyl)amino)methyl)phenyl)thiazole-4-carboxylate (8.3)

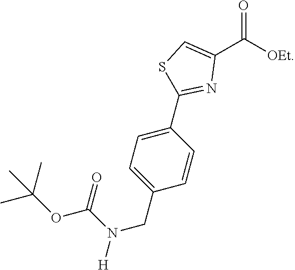 [SMILES: CCOC(=O)c1csc(-c2ccc(CNC(=O)OC(C)(C)C)cc2)n1]

Ethyl 2-bromothiazole-4-carboxylate (0.504 g, 2.13 mmole) was dissolved in 1,4-dioxane (4 mL). Tert-butyl (4-(4,4,5,5-tetramethyl-1,3,2-dioxaborolan-2-yl)benzyl)carbamate (0.862 g, 2.59 mmole) was added followed by potassium carbonate (2M aq. solution, 2.10 mL, 4.20 mmole) and bis(triphenylphosphine)palladium(II) chloride (0.149 g, 0.212 mmole). The mixture was heated to 80° C. for 20 hours. The mixture was cooled to RT and water (50 mL) was added. The mixture was extracted with ethyl acetate (3×15 mL). The combined organics were dried over magnesium sulfate, filtered, and concentrated. The crude residue was purified by silica gel chromatography (Isco CombiPrep, 12 g Silicycle column, 10-30% ethyl acetate/hexane gradient) to provide ethyl 2-(4-(((tert-butoxycarbonyl)amino)methyl)phenyl)thiazole-4-carboxylate as a yellow solid (0.400 g, 52%). ¹H NMR (400 MHz, Chloroform-d) δ 8.15 (s, 1H), 8.01-7.92 (m, 2H), 7.41-7.32 (m, 2H), 4.92 (s, 1H), 4.51-4.40 (m, 2H), 4.35 (dd, J=9.9, 6.7 Hz, 2H), 1.51-1.45 (m, 9H), 1.45-1.40 (m, 3H).

### 2-(4-(((Tert-butoxycarbonyl)amino)methyl)phenyl)thiazole-4-carboxylic acid (8.4)

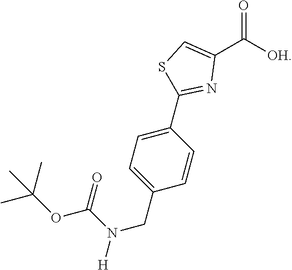 [SMILES: CC(C)(C)OC(=O)NCc1ccc(-c2nc(C(=O)O)cs2)cc1]

Using the procedure described for Example 4.3, ethyl 2-(4-(((tert-butoxycarbonyl)amino)methyl)phenyl)thiazole-4-carboxylate (0.400 g, 1.10 mmole) was converted to 2-(4-(((tert-butoxycarbonyl)amino)methyl)phenyl)thiazole-4-carboxylic acid (0.338 g, 92%) as a white solid. ¹H NMR (400 MHz, DMSO-d₆) δ 13.10 (s, 1H), 8.48 (s, 1H), 8.00-7.84 (m, 2H), 7.48 (t, J=6.2 Hz, 1H), 7.38 (d, J=8.2 Hz, 2H), 4.19 (d, J=6.2 Hz, 2H), 1.40 (s, 9H).

### Methyl (2-(4-(((tert-butoxycarbonyl)amino)methyl)phenyl)thiazole-4-carbonyl)-L-serinate (8.5)

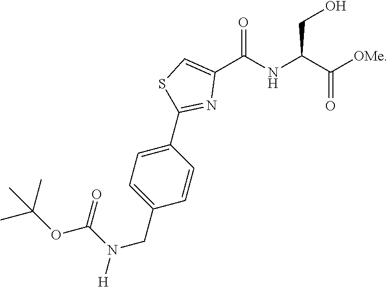 [SMILES: COC(=O)[C@H](CO)NC(=O)c1csc(-c2ccc(CNC(=O)OC(C)(C)C)cc2)n1]

Using the procedure described for Example 1.4, 2-(4-(((tert-butoxycarbonyl)amino)methyl)phenyl)thiazole-4-carboxylic acid (0.338 g, 1.01 mmole) was converted to methyl (2-(4-(((tert-butoxycarbonyl)amino)methyl)phenyl)thiazole-4-carbonyl)-L-serinate (0.336 g, 76%) as a white solid. ¹H NMR (400 MHz, Chloroform-d) δ 8.26 (d, J=7.5 Hz, 1H), 8.11 (s, 1H), 7.92 (d, J=8.1 Hz, 2H), 7.37 (d, J=8.0 Hz, 2H), 4.95 (s, 1H), 4.88 (dt, J =7.5, 3.8 Hz, 1H), 4.37 (d, J=6.1 Hz, 2H), 4.17-4.05 (m, 2H), 3.85 (s, 3H), 1.48 (s, 9H).

### N-(2-(4-(((Tert-butoxycarbonyl)amino)methyl)-phenyl)thiazole-4-carbonyl)-O-(tert-butyldimethylsilyl)-L-serine (8.6)

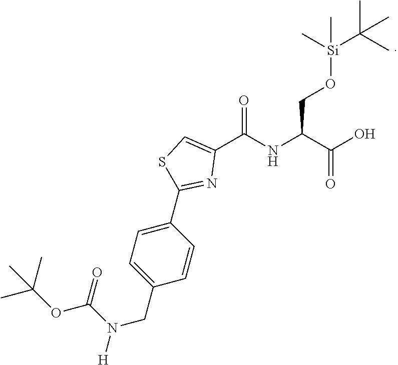 [SMILES: CC(C)(C)OC(=O)NCc1ccc(-c2nc(C(=O)N[C@@H](CO[Si](C)(C)C(C)(C)C)C(=O)O)cs2)cc1]

Using the procedure described for Example 4.2 and 4.3, methyl (2-(4-(((tert-butoxycarbonyl)amino)methyl)phenyl)thiazole-4-carbonyl)-L-serinate (0.336 g, 0.772 mmole) was converted to N-(2-(4-(((tert-butoxycarbonyl)amino)methyl)-phenyl)thiazole-4-carbonyl)-O-(tert-butyldimethylsilyl)-L-serine (0.334 g, 81%) as a white solid. ¹H NMR (400 MHz, DMSO-d₆) δ8.41 (s, 1H), 8.23 (d, J=8.6 Hz, 1H), 8.04-7.94 (m, 2H), 7.53 (t, J=6.2 Hz, 1H), 7.43 (d, J=8.0 Hz, 2H), 4.63 (dt, J=8.6, 3.5 Hz, 1H), 4.24 (d, J=6.2 Hz, 2H), 4.16 (dd, J=10.3, 3.4 Hz, 1H), 3.99 (dd, J=10.3, 3.7 Hz, 1H), 1.45 (s, 9H), 0.93 (s, 9H), 0.10 (s, 3H), 0.08 (s, 3H).

### Methyl N-(2-(4-(((tert-butoxycarbonyl)amino)methyl)-phenyl)thiazole-4-carbonyl)-O-(tert-butyldimethylsilyl)-L-seryl-L-serinate (8.7)

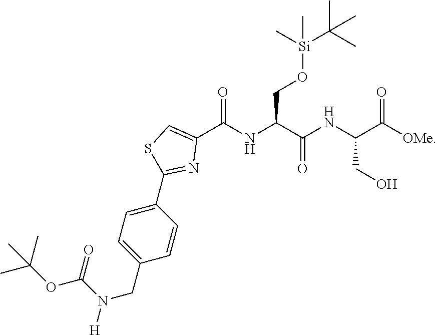 [SMILES: COC(=O)[C@H](CO)NC(=O)[C@H](CO[Si](C)(C)C(C)(C)C)NC(=O)c1csc(-c2ccc(CNC(=O)OC(C)(C)C)cc2)n1]

Using the procedure described for Example 1.4, N-(2-(4-(((tert-butoxycarbonyl)amino)methyl)-phenyl)thiazole-4-carbonyl)-O-(tert-butyldimethylsilyl)-L-serine (0.334 g, 0.623 mmole) was converted to methyl N-(2-(4-(((tert-butoxycarbonyl)amino)methyl)-phenyl)thiazole-4-carbonyl)-O-(tert-butyldimethylsilyl)-L-seryl-L-serinate (0.263 g, 66%) as a colorless gel. ¹H NMR (400 MHz, CDCl₃) δ 8.26 (d, J=7.0 Hz, 1H), 8.09 (s, 1H), 7.95-7.83 (m, 2H), 7.47 (d, J=7.3 Hz, 1H), 7.35 (d, J=8.2 Hz, 2H), 4.95 (s, 1H), 4.70 (dt, J=7.3, 3.7 Hz, 1H), 4.66 (dt, J=6.7, 3.3 Hz, 1H), 4.37 (d, J=6.1 Hz, 2H), 4.21 (dd, J=9.9, 4.1 Hz, 1H), 4.04-3.94 (m, 2H), 3.88-3.81 (m, 1H), 3.79 (s, 3H), 1.48 (s, 9H), 0.95 (s, 9H), 0.14 (s, 3H), 0.13 (s, 3H).

### Methyl 2-(2-(2-(4-(((tert-butoxycarbonyl)amino)methyl)phenyl)thiazole-4-carboxamido)acrylamido)acrylate (8)

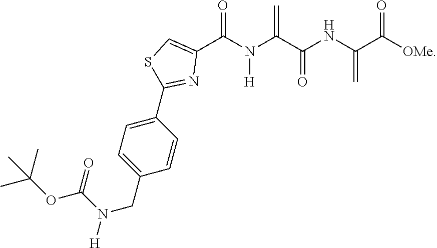 [SMILES: C=C(NC(=O)c1csc(-c2ccc(CNC(=O)OC(C)(C)C)cc2)n1)C(=O)NC(=C)C(=O)OC]

Using the procedure described for Compound 2, methyl N-(2-(4-(((tert-butoxycarbonyl)amino)methyl)-phenyl)thiazole-4-carbonyl)-O-(tert-butyldimethylsilyl)-L-seryl-L-serinate (0.263 g, 0.413 mmole) was converted to methyl 2-(2-(2-(4-(((tert-butoxycarbonyl)amino)methyl)phenyl)thiazole-4-carboxamido)acrylamido)acrylate (0.0461 g, 15%) as a white solid. ¹H NMR (400 MHz, CDCl₃) δ 10.02 (s, 1H), 8.55 (s, 1H), 8.14 (s, 1H), 8.03-7.93 (m, 2H), 7.38 (d, J=8.2 Hz, 2H), 6.78 (d, J=2.2 Hz, 1H), 6.71 (d, J=0.5 Hz, 1H), 6.03 (d, J=1.3 Hz, 1H), 5.50 (t, J=1.9 Hz, 1H), 4.92 (s, 1H), 4.38 (d, J=6.1 Hz, 2H), 3.91 (s, 3H), 1.48 (s, 9H).

### Compound 9: Tert-butyl 4-(4-((3-((3-methoxy-3-oxoprop-1-en-2-yl)amino)-3-oxoprop-1-en-2-yl)carbamoyl)thiazol-2-yl)-3,6-dihydropyridine-1(2H)-carboxylate

### Tert-butyl (S)-4-(4-((3-((tert-butyldimethylsilyl)oxy)-1-methoxy-1-oxopropan-2-yl)carbamoyl)thiazol-2-yl)-3,6-dihydropyridine-1(2H)-carboxylate (9.1)

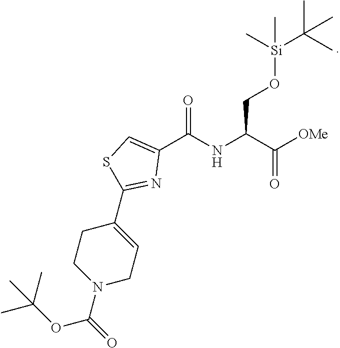 [SMILES: COC(=O)[C@H](CO[Si](C)(C)C(C)(C)C)NC(=O)c1csc(C2=CCN(C(=O)OC(C)(C)C)CC2)n1]

Methyl N-(2-bromothiazole-4-carbonyl)-O-(tert-butyldimethylsilyl)-L-serinate (0.511 g, 1.21 mmole) was dissolved in 1,4-dioxane (4 mL). Tert-butyl 4-(4,4,5,5-tetramethyl-1,3,2-dioxaborolan-2-yl)-3,6-dihydropyridine-1(2H)-carboxylate (0.440 g, 1.42 mmole) was added followed by potassium carbonate (2M aqueous solution, 1.20 mL, 2.40 mmole) and bis(triphenylphosphine)palladium(II) chloride (0.086 g, 0.123 mmole). The mixture was heated to 80° C. for 18 hours. The mixture was cooled to RT and poured into water (25 mL), then was extracted with ethyl acetate (3×10 mL). The combined organics were dried over magnesium sulfate, filtered, and concentrated. The crude residue was purified by silica gel chromatography to provide tert-butyl (S)-4-(4-((3-((tert-butyldimethylsilyl)oxy)-1-methoxy-1-oxopropan-2-yl)carbamoyl)thiazol-2-yl)-3,6-dihydropyridine-1(2H)-carboxylate as a red gel (0.556 g, 87%). ¹H NMR (400 MHz, CDCl₃) δ 8.09 (t, J=8.6 Hz, 1H), 7.99 (s, 1H), 6.57 (s, 1H), 4.82 (ddd, J=8.7, 3.3, 2.6 Hz, 1H), 4.18 (dd, J=10.0, 2.6 Hz, 1H), 4.13 (dd, J=8.5, 5.8 Hz, 3H), 3.92 (dd, J=10.1, 3.3 Hz, 1H), 3.77 (s, 3H), 3.72-3.54 (m, 2H), 2.70 (s, 2H), 1.50 (s, 9H), 0.89 (s, 9H), 0.05 (s, 3H).

### N-(2-(1-(Tert-butoxycarbonyl)-1,2,3,6-tetrahydropyridin-4-yl)thiazole-4-carbonyl)-O-(tert-butyldimethylsilyl)-L-serine (9.2)

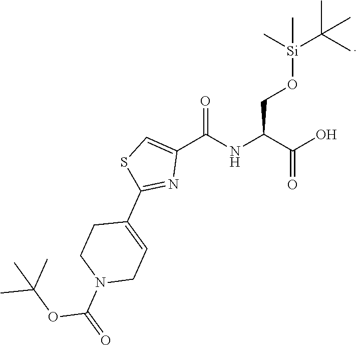 [SMILES: CC(C)(C)OC(=O)N1CC=C(c2nc(C(=O)N[C@@H](CO[Si](C)(C)C(C)(C)C)C(=O)O)cs2)CC1]

Tert-butyl (S)-4-(4-((3-((tert-butyldimethylsilyl)oxy)-1-methoxy-1-oxopropan-2-yl)carbamoyl)thiazol-2-yl)-3,6-dihydropyridine-1(2H)-carboxylate (0.556 g, 1.06 mmole) was dissolved in 4/1/1 THF/methanol/water (6 mL). Lithium hydroxide (0.086 g, 3.59 mmole) was added and the mixture was stirred at RT for 2 hours. Water (25 mL) was added and the solution was treated with 1N aq. HCl to pH=4. The slurry was extracted with ethyl acetate (3×10 mL). The combined organics were dried over sodium sulfate, filtered, and concentrated to provide N-(2-(1-(tert-butoxycarbonyl)-1,2,3,6-tetrahydropyridin-4-yl)thiazole-4-carbonyl)-O-(tert-butyldimethylsilyl)-L-serine as an orange gel (0.501 g, 92%). ¹H NMR (400 MHz, DMSO) δ 8.31 (s, 1H), 8.06 (d, J=8.7 Hz, 1H), 6.75 (d, J=14.0 Hz, 1H), 4.59 (dt, J=8.6, 3.4 Hz, 1H), 4.15-4.10 (m, 3H), 3.95 (dd, J=10.3, 3.5 Hz, 2H), 3.64-3.54 (m, 2H), 2.64 (s, 2H), 1.47 (s, 9H), 0.90 (d, J=3.7 Hz, 9H), 0.08 (s, 3H), 0.06 (s, 3H)

### Tert-butyl 4-(4-(((4S,7S)-4-(hydroxymethyl)-10,10,11,11-tetramethyl-3,6-dioxo-2,9-dioxa-5-aza-10-siladodecan-7-yl)carbamoyl)thiazol-2-yl)-3,6-dihydropyridine-1(2H)-carboxylate (9.3)

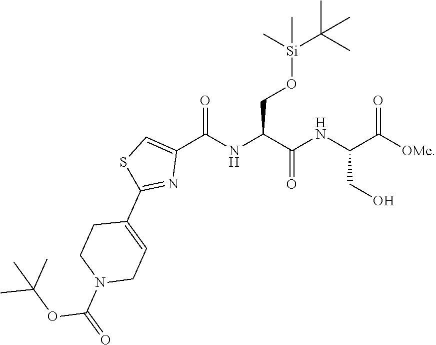 [SMILES: COC(=O)[C@H](CO)NC(=O)[C@H](CO[Si](C)(C)C(C)(C)C)NC(=O)c1csc(C2=CCN(C(=O)OC(C)(C)C)CC2)n1]

Using the procedure for Example 2.7, N-(2-(1-(tert-butoxycarbonyl)-1,2,3,6-tetrahydropyridin-4-yl)thiazole-4-carbonyl)-O-(tert-butyldimethylsilyl)-L-serine (0.501 g, 0.979 mmole) was converted to tert-butyl 4-(4-(((4S,7S)-4-(hydroxymethyl)-10,10,11,11-tetramethyl-3,6-dioxo-2,9-dioxa-5-aza-10-siladodecan-7-yl)carbamoyl)thiazol-2-yl)-3,6-dihydropyridine-1(2H)-carboxylate (0.264 g, 44%) as a white solid. ¹H NMR (400 MHz, CDCl₃) δ 8.01 (d, J=5.3 Hz, 1H), 7.46 (dd, J=13.4, 7.1 Hz, 1H), 6.57 (d, J=3.6 Hz, 1H), 4.68 (dt, J=6.9, 3.4 Hz, 1H), 4.66-4.55 (m, 1H), 4.22-4.09 (m, 2H), 3.98 (d, J=3.6 Hz, 2H), 3.82 (dt, J=10.5, 3.5 Hz, 1H), 3.80-3.73 (m, 3H), 3.61 (q, J=7.2, 6.7 Hz, 1H), 2.69 (s, 1H), 1.50 (d, J=5.6 Hz, 9H), 0.92 (s, 9H), 0.12 (d, J=3.6 Hz, 6H).

### Tert-butyl 4-(4-((3-((3-methoxy-3-oxoprop-1-en-2-yl)amino)-3-oxoprop-1-en-2-yl)carbamoyl)thiazol-2-yl)-3,6-dihydropyridine-1(2H)-carboxylate (9)

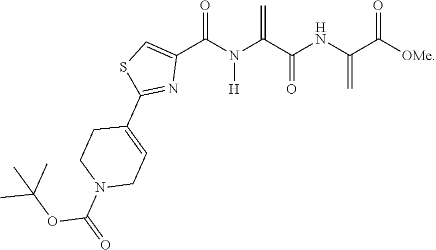 [SMILES: C=C(NC(=O)c1csc(C2=CCN(C(=O)OC(C)(C)C)CC2)n1)C(=O)NC(=C)C(=O)OC]

Using the procedure for Compound 2, tert-butyl 4-(4-(((4S,7S)-4-(hydroxymethyl)-10,10,11,11-tetramethyl-3,6-dioxo-2,9-dioxa-5-aza-10-siladodecan-7-yl)carbamoyl)thiazol-2-yl)-3,6-dihydropyridine-1(2H)-carboxylate (0.264 g, 0.431 mmole) was converted to tert-butyl 4-(4-((3-((3-methoxy-3-oxoprop-1-en-2-yl)amino)-3-oxoprop-1-en-2-yl)carbamoyl)thiazol-2-yl)-3,6-dihydropyridine-1(2H)-carboxylate (0.052 g, 27%) as a white solid. ¹H NMR (400 MHz, CDCl₃) δ 9.91 (s, 1H), 8.53 (s, 1H), 8.04 (s, 1H), 6.76 (d, J=2.2 Hz, 1H), 6.69 (s, 1H), 6.66-6.60 (m, 1H), 6.02 (d, J=1.3 Hz, 1H), 5.48 (t, J=1.9 Hz, 1H), 4.20-4.11 (m, 2H), 3.90 (s, 3H), 3.65 (t, J=5.7 Hz, 2H), 2.74 (qd, J=4.6, 4.1, 2.4 Hz, 2H), 1.49 (s, 9H).

### Compound 10: Methyl 2-(2-(2-(4-((2-methoxyethyl)carbamoyl)phenyl)thiazole-4-carboxamido)acrylamido)acrylate

### N-(2-Methoxyethyl)-4-(4,4,5,5-tetramethyl-1,3,2-dioxaborolan-2-yl)benzamide (10.1)

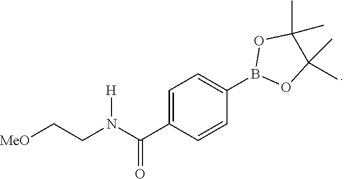 [SMILES: COCCNC(=O)c1ccc(B2OC(C)(C)C(C)(C)O2)cc1]

4-(4,4,5,5-Tetramethyl-1,3,2-dioxaborolan-2-yl)benzoic acid (0.503 g, 2.03 mmole) was dissolved in DCM (4 mL). 2-Methoxyethylamine (0.210 mL, 2.42 mmole) was added followed by N,N-diisopropylethylamine (0.700 mL, 4.02 mmole) and pyBOP (1.268 g, 2.44 mmole). The mixture was stirred at RT for 6 hours. Water (25 mL) was added and the mixture was extracted with DCM (3×10 mL). The combined organics were dried over sodium sulfate, filtered, and concentrated. The crude residue was purified by silica gel chromatography to provide N-(2-methoxyethyl)-4-(4,4,5,5-tetramethyl-1,3,2-dioxaborolan-2-yl)benzamide as a colorless oil (0.561 g, 91%). ¹H NMR (400 MHz, CDCl₃) δ 7.90-7.83 (m, 2H), 7.80-7.72 (m, 2H), 6.54 (d, J=6.2 Hz, 1H), 3.71-3.62 (m, 2H), 3.57 (dd, J=5.4, 4.3 Hz, 2H), 3.39 (s, 3H), 1.36 (s, 12H).

### Methyl O-(tert-butyldimethylsilyl)-N-(2-(4-((2-methoxyethyl)carbamoyl)phenyl)thiazole-4-carbonyl)-L-serinate (10.2)

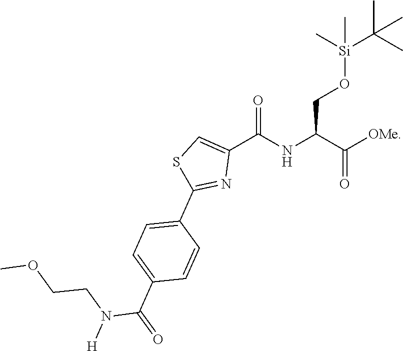 [SMILES: COCCNC(=O)c1ccc(-c2nc(C(=O)N[C@@H](CO[Si](C)(C)C(C)(C)C)C(=O)OC)cs2)cc1]

Methyl N-(2-bromothiazole-4-carbonyl)-O-(tert-butyldimethylsilyl)-L-serinate (0.656 g, 1.55 mmole) and N-(2-methoxyethyl)-4-(4,4,5,5-tetramethyl-1,3,2-dioxaborolan-2-yl)benzamide (0.561 g, 1.84 mmole) were dissolved in 1,4-dioxane (3 mL). Potassium carbonate (2M aqueous solution, 1.50 mL, 3.00 mmole) and bis(triphenylphosphine)palladium(II) chloride (0.114 g, 0.162 mmole) were added and the mixture was heated to 80° C. for 24 hours. The mixture was cooled to RT and stirred for 36 hours. Water (25 mL) was added and the mixture was extracted with ethyl acetate (3×10 mL). The combined organics were dried over magnesium sulfate, filtered, and concentrated. The crude residue was purified by silica gel chromatography to provide methyl O-(tert-butyldimethylsilyl)-N-(2-(4-((2-methoxyethyl)carbamoyl)phenyl)thiazole-4-carbonyl)-L-serinate as an orange oil (0.370 g, 46%). ¹H NMR (400 MHz, CDCl₃) δ 8.22 (d, J=8.7 Hz, 1H), 8.16 (s, 1H), 8.08-8.00 (m, 2H), 7.92-7.82 (m, 2H), 6.59 (t, J=5.2 Hz, 1H), 4.90-4.83 (m, 1H), 4.22 (dd, J=10.1, 2.6 Hz, 1H), 4.01-3.90 (m, 1H), 3.79 (s, 3H), 3.74-3.65 (m, 2H), 3.60 (dd, J=5.4, 4.3 Hz, 2H), 3.42 (s, 3H), 0.93 (s, 9H), 0.08 (s, 3H), 0.06 (s, 3H).

### O-(Tert-butyldimethylsilyl)-N-(2-(4-((2-methoxyethyl)carbamoyl)phenyl)thiazole-4-carbonyl)-L-serine (10.3)

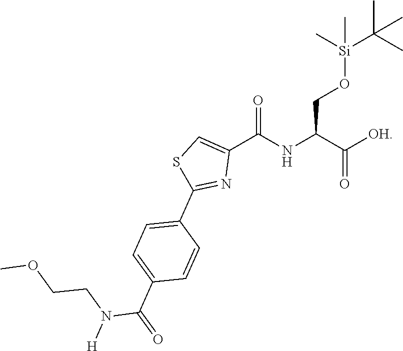 [SMILES: COCCNC(=O)c1ccc(-c2nc(C(=O)N[C@@H](CO[Si](C)(C)C(C)(C)C)C(=O)O)cs2)cc1]

Using the procedure described in Example 9.2, methyl O-(tert-butyldimethylsilyl)-N-(2-(4-((2-methoxyethyl)carbamoyl)phenyl)thiazole-4-carbonyl)-L-serinate (0.370 g, 0.709 mmole) was converted to 0-(tert-butyldimethylsilyl)-N-(2-(4-((2-methoxyethyl)carbamoyl)phenyl)thiazole-4-carbonyl)-L-serine as an orange solid (0.287 g, 80%). ¹H NMR (400 MHz, DMSO-d₆) δ 8.76 (t, J=4.8 Hz, 1H), 8.49 (d, J=7.4 Hz, 1H), 8.26 (d, J=8.6 Hz, 1H), 8.16-8.10 (m, 2H), 8.10-8.01 (m, 2H), 4.65 (dt, J=8.6, 3.6 Hz, 1H), 4.16 (dd, J=10.3, 3.6 Hz, 1H), 4.01 (dd, J=10.3, 3.7 Hz, 1H), 3.57-3.45 (m, 5H), 3.44-3.35 (m, 2H), 0.93 (s, 9H), 0.11 (s, 3H), 0.09 (s, 3H).

### Methyl O-(tert-butyldimethylsilyl)-N-(2-(4-((2-methoxyethyl)carbamoyl)phenyl)thiazole-4-carbonyl)-L-seryl-L-serinate (10.4)

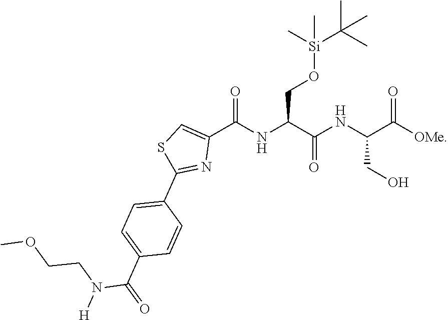 [SMILES: COCCNC(=O)c1ccc(-c2nc(C(=O)N[C@@H](CO[Si](C)(C)C(C)(C)C)C(=O)N[C@@H](CO)C(=O)OC)cs2)cc1]

Using the procedure described for Example 2.7, 0-(tert-butyldimethylsilyl)-N-(2-(4-((2-methoxyethyl)carbamoyl)phenyl)thiazole-4-carbonyl)-L-serine (0.287 g, 0.565 mmole) was converted to methyl O-(tert-butyldimethylsilyl)-N-(2-(4-((2-methoxyethyl)carbamoyl)phenyl)thiazole-4-carbonyl)-L-seryl-L-serinate (0.344 g, 100%) as a white solid. ¹H NMR (400 MHz, CDCl₃) δ 8.25 (d, J=6.9 Hz, 1H), 8.17 (d, J=2.1 Hz, 1H), 8.06-7.99 (m, 2H), 7.90-7.82 (m, 2H), 7.44 (d, J=7.0 Hz, 1H), 6.57 (s, 1H), 4.70 (dt, J=7.4, 3.8 Hz, 1H), 4.67-4.60 (m, 1H), 4.22 (dd, J=9.8, 4.1 Hz, 1H), 4.04-3.95 (m, 2H), 3.89-3.81 (m, 1H), 3.79 (d, J=6.8 Hz, 3H), 3.73-3.65 (m, 2H), 3.60 (dd, J=5.4, 4.2 Hz, 2H), 3.42 (s, 3H), 0.96 (s, 9H), 0.16 (s, 3H), 0.15 (s, 3H).

### Methyl 2-(2-(2-(4-((2-methoxyethyl)carbamoyl)phenyl)thiazole-4-carboxamido)acrylamido)acrylate (10)

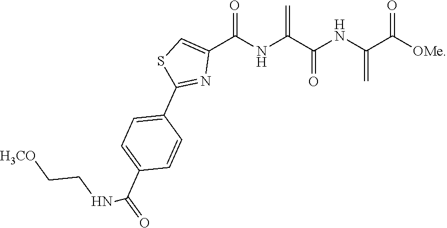 [SMILES: C=C(NC(=O)c1csc(-c2ccc(C(=O)NCCOC)cc2)n1)C(=O)NC(=C)C(=O)OC]

Using the procedure described for Compound 2, methyl (S)-2-(3-hydroxy-2-(2-(4-((2-methoxyethyl)carbamoyl)phenyl)-thiazole-4-carboxamido)propanamido)acrylate (0.212 g, 0.445 mmole) was converted to methyl 2-(2-(2-(4-((2-methoxyethyl)carbamoyl)phenyl)thiazole-4-carboxamido)acrylamido)acrylate as a white solid (0.086 g, 33%).

¹H NMR (400 MHz, CDCl₃) δ 10.02 (s, 1H), 8.56 (s, 1H), 8.20 (s, 1H), 8.13-8.05 (m, 2H), 7.94-7.83 (m, 2H), 6.79 (d, J=2.2 Hz, 1H), 6.72 (s, 1H), 6.58 (d, J=5.7 Hz, 1H), 6.04 (d, J=1.3 Hz, 1H), 5.52 (t, J=1.9 Hz, 1H), 3.91 (s, 3H), 3.75-3.65 (m, 2H), 3.60 (dd, J=5.4, 4.3 Hz, 2H), 3.42 (s, 3H).

### Compound 11: Methyl (R)-2-(2-(2-(3-((tert-butoxycarbonyl)amino)piperidin-1-yl)thiazole-4-carboxamido)acrylamido)acrylate

### Ethyl (R)-2-(3-((tert-butoxycarbonyl)amino)piperidin-1-yl)thiazole-4-carboxylate (11.1)

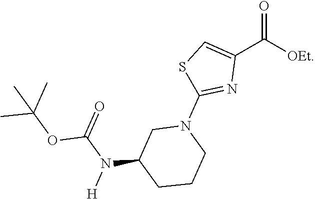 [SMILES: CCOC(=O)c1csc(N2CCC[C@@H](NC(=O)OC(C)(C)C)C2)n1]

Ethyl 2-bromothiazole-4-carboxylate (1.007 g, 4.27 mmole) was dissolved in N,N-dimethylacetamide (8 mL). Tert-butyl (R)-piperidin-3-ylcarbamate (0.900 g, 4.49 mmole) was added followed by triethylamine (0.660 mL, 4.71 mmole). The solution was heated to 70° C. for 3 days, then was cooled to RT. Water (40 mL) was added and the mixture was extracted with ethyl acetate (3×15 mL). The combined organics were dried over magnesium sulfate, filtered, and concentrated. The crude residue was purified by silica gel chromatography to provide ethyl (R)-2-(3-((tert-butoxycarbonyl)amino)piperidin-1-yl)thiazole-4-carboxylate as a yellow solid (1.032 g, 68%). ¹H NMR (400 MHz, Chloroform-d) δ 7.44 (s, 1H), 4.69 (s, 1H), 4.35 (q, J=7.1 Hz, 2H), 3.80 (s, 1H), 3.70 (d, J=12.8 Hz, 1H), 3.61 (s, 1H), 3.46 (s, 1H), 3.29 (s, 1H), 1.97-1.86 (m, 1H), 1.86-1.76 (m, 1H), 1.76-1.65 (m, 1H), 1.57 (d, J=24.5 Hz, 1H), 1.45 (s, 9H), 1.37 (t, J=7.1 Hz, 3H).

### (R)-2-(3-((Tert-butoxycarbonyl)amino)piperidin-1-yl)thiazole-4-carboxylic acid (11.2)

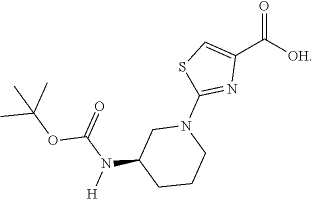 [SMILES: CC(C)(C)OC(=O)N[C@@H]1CCCN(c2nc(C(=O)O)cs2)C1]

Using the procedure described for Example 1.3, ethyl (R)-2-(3-((tert-butoxycarbonyl)amino)piperidin-1-yl)thiazole-4-carboxylate (1.032 g, 2.90 mmole) was converted to (R)-2-(3-((tert-butoxycarbonyl)amino)piperidin-1-yl)thiazole-4-carboxylic acid (0.847 g, 89%) as a yellow solid. ¹H NMR (400 MHz, CDCl₃) δ 7.51 (s, 1H), 4.66 (s, 1H), 3.86-3.71 (m, 2H), 3.59 (s, 1H), 3.40 (s, 1H), 3.26 (s, 1H), 1.95 (d, J=4.0 Hz, 1H), 1.85 (ddt, J=10.4, 7.2, 3.6 Hz, 1H), 1.72 (ddq, J=13.1, 8.6, 4.0 Hz, 1H), 1.57 (s, 1H), 1.46 (s, 9H).

### Methyl (2-((R)-3-((tert-butoxycarbonyl)amino)piperidin-1-yl)thiazole-4-carbonyl)-L-serinate (11.3)

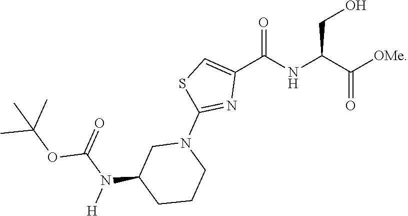 [SMILES: COC(=O)[C@H](CO)NC(=O)c1csc(N2CCC[C@@H](NC(=O)OC(C)(C)C)C2)n1]

Using the procedure described for Example 1.4, (R)-2-(3-((tert-butoxycarbonyl)amino)piperidin-1-yl)thiazole-4-carboxylic acid (0.400 g, 1.22 mmole) was converted to methyl (2-((R)-3-((tert-butoxycarbonyl)amino)piperidin-1-yl)thiazole-4-carbonyl)-L-serinate (0.547 g, 100%) as a white solid. ¹H NMR (400 MHz, CDCl₃) δ 8.05 (d, J=7.8 Hz, 1H), 7.40 (s, 1H), 4.79 (dt, J=7.6, 3.8 Hz, 1H), 4.65 (s, 1H), 4.09-3.99 (m, 2H), 3.89 (d, J=12.6 Hz, 1H), 3.81 (s, 4H), 3.54 (s, 1H), 3.39 (s, 1H), 3.13 (dd, J=12.5, 7.7 Hz, 1H), 1.93 (dd, J=7.8, 4.5 Hz, 1H), 1.71 (dp, J=13.5, 4.5 Hz, 1H), 1.56 (d, J=9.0 Hz, 1H), 1.45 (s, 9H).

### N-(2-((R)-3-((Tert-butoxycarbonyl)amino)piperidin-1-yl)thiazol-4-carbonyl)-O-(tert-butyldimethylsilyl)-L-serine (11.4)

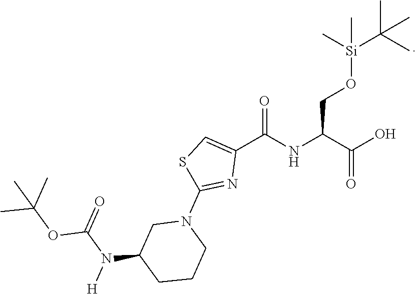 [SMILES: CC(C)(C)OC(=O)N[C@@H]1CCCN(c2nc(C(=O)N[C@@H](CO[Si](C)(C)C(C)(C)C)C(=O)O)cs2)C1]

Using the procedure described for Example 4.2 and 4.3, methyl (2-((R)-3-((tert-butoxycarbonyl)amino)piperidin-1-yl)thiazole-4-carbonyl)-L-serinate (0.456 g, 1.06 mmole) was converted to N-(2-((R)-3-((tert-butoxycarbonyl)amino)piperidin-1-yl)thiazol-4-carbonyl)-O-(tert-butyldimethylsilyl)-L-serine (0.510 g, 91%) as a colorless oil. ¹H NMR (400 MHz, CDCl₃) δ 7.95 (d, J=7.6 Hz, 1H), 7.42 (s, 1H), 4.78-4.64 (m, 2H), 4.22 (dd, J=10.1, 3.6 Hz, 1H), 3.90 (dd, J=10.1, 4.7 Hz, 1H), 3.80 (s, 1H), 3.69 (s, 1H), 3.54 (s, 1H), 3.39 (s, 1H), 3.24 (s, 1H), 1.90 (d, J=17.3 Hz, 1H), 1.71 (dt, J=9.0, 4.3 Hz, 1H), 1.45 (s, 10H), 0.91 (d, J=5.4 Hz, 9H), 0.11 (s, 3H), 0.09 (s, 3H).

### Methyl N-(2-((R)-3-((tert-butoxycarbonyl)amino)piperidin-1-yl)thiazole-4-carbonyl)-O-(tert-butyldimethylsilyl)-L-seryl-L-serinate (11.5)

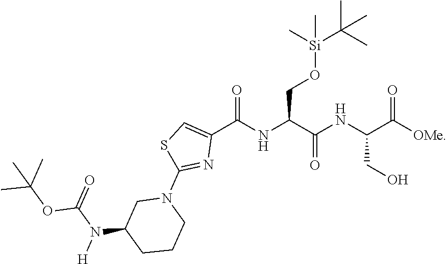 [SMILES: COC(=O)[C@H](CO)NC(=O)[C@H](CO[Si](C)(C)C(C)(C)C)NC(=O)c1csc(N2CCC[C@@H](NC(=O)OC(C)(C)C)C2)n1]

Using the procedure described for Example 2.7, N-(2-((R)-3-((tert-butoxycarbonyl)amino)piperidin-1-yl)thiazol-4-carbonyl)-O-(tert-butyldimethylsilyl)-L-serine (0.510 g, 0.965 mmole) was converted to methyl N-(2-((R)-3-((tert-butoxycarbonyl)amino)piperidin-1-yl)thiazole-4-carbonyl)-O-(tert-butyldimethylsilyl)-L-seryl-L-serinate (0.401 g, 66%) as a pale yellow oil. ¹H NMR (400 MHz, CDCl₃) δ 7.97 (d, J=7.1 Hz, 1H), 7.40 (s, 2H), 4.67 (td, J=8.3, 7.3, 4.8 Hz, 2H), 4.62-4.50 (m, 1H), 4.19-4.14 (m, 1H), 3.97 (d, J=3.7 Hz, 2H), 3.85-3.79 (m, 1H), 3.77 (d, J=2.6 Hz, 4H), 3.68 (s, 1H), 3.54 (s, 1H), 3.40 (s, 1H), 3.32-3.15 (m, 1H), 1.99-1.87 (m, 1H), 1.71 (d, J=9.8 Hz, 1H), 1.45 (s, 9H), 0.91 (s, 9H), 0.12 (s, 3H), 0.10 (s, 3H).

### Methyl (R)-2-(2-(2-(3-((tert-butoxycarbonyl)amino)piperidin-1-yl)thiazole-4-carboxamido)acrylamido)acrylate (11)

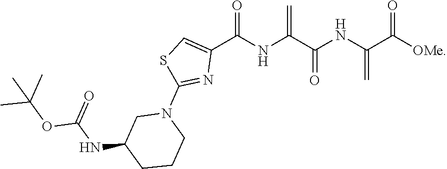 [SMILES: C=C(NC(=O)c1csc(N2CCC[C@@H](NC(=O)OC(C)(C)C)C2)n1)C(=O)NC(=C)C(=O)OC]

Using the procedure described for Compound 2, methyl 2-((S)-2-(2-((R)-3-((tert-butoxycarbonyl)amino)piperidin-1-yl)thiazole-4-carboxamido)-3-hydroxypropanamido)acrylate (0.257 g, 0.517 mmole) was converted to methyl (R)-2-(2-(2-(3-((tert-butoxycarbonyl)amino)piperidin-1-yl)thiazole-4-carboxamido)acrylamido)acrylate as a white solid (0.102 g, 33%). ¹H NMR (400 MHz, CDCl₃) δ 9.71 (s, 1H), 8.50 (s, 1H), 7.43 (s, 1H), 6.71 (d, J=2.1 Hz, 1H), 6.66 (s, 1H), 6.00 (d, J=1.3 Hz, 1H), 5.43 (t, J=1.9 Hz, 1H), 4.71 (s, 1H), 3.89 (s, 3H), 3.86-3.77 (m, 1H), 3.74-3.64 (m, 1H), 3.59 (s, 1H), 3.47 (s, 1H), 3.30 (s, 1H), 1.98-1.78 (m, 2H), 1.78-1.67 (m, 1H), 1.45 (s, 9H).

### Compound 12: Methyl 2-(2-(2-(4-((tert-butoxycarbonyl)amino)piperidin-1-yl)thiazole-4-carboxamido)acrylamido)acrylate

### Ethyl 2-(4-((tert-butoxycarbonyl)amino)piperidin-1-yl)thiazole-4-carboxylate (12.1)

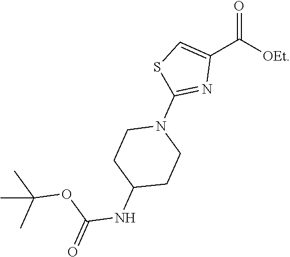 [SMILES: CCOC(=O)c1csc(N2CCC(NC(=O)OC(C)(C)C)CC2)n1]

Ethyl 2-bromothiazole-4-carboxylate (3.007 g, 12.7 mmole) was dissolved in N,N-dimethylacetamide (13 mL). Tert-butyl piperidin-4-yl carbamate (2.668 g, 13.3 mmole) was added followed by trimethylamine (2.00 mL, 14.3 mmole), and the mixture was heated to 80° C. for 24 hours. The solution was cooled to RT and water (50 mL) was added. The mixture was extracted with ethyl acetate (3×20 mL). The combined organics were dried over magnesium sulfate, filtered, and concentrated. The crude residue was purified by silica gel chromatography to provide ethyl 2-(4-((tert-butoxycarbonyl)amino)piperidin-1-yl)thiazole-4-carboxylate as a white solid (3.562 g, 79%). ¹H NMR (400 MHz, CDCl₃): δ 7.43 (s, 1H), 4.48 (s, 1H), 4.35 (q, J =7.1 Hz, 2H), 4.05-3.92 (m, 2H), 3.68 (s, 1H), 3.15 (ddd, J=13.2, 11.6, 3.0 Hz, 2H), 2.05 (m, 2H), 1.56-1.47 (m, 2H), 1.45 (s, 9H), 1.37 (t, J=7.1 Hz, 3H).

### 2-(4-((Tert-butoxycarbonyl)amino)piperidin-1-yl)thiazole-4-carboxylic acid (12.2)

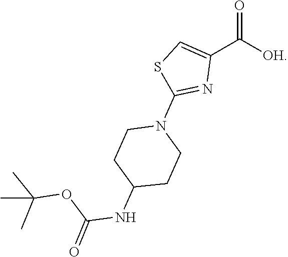 [SMILES: CC(C)(C)OC(=O)NC1CCN(c2nc(C(=O)O)cs2)CC1]

The procedure described in Example 1.3 was used to convert ethyl 2-(4-((tert-butoxycarbonyl)amino)piperidin-1-yl)thiazole-4-carboxylate (3.562 g, 10.0 mmole) to 2-(4-((tert-butoxycarbonyl)amino)piperidin-1-yl)thiazole-4-carboxylic acid as a yellow solid (2.994 g, 91%). ¹H NMR (400 MHz, CDCl₃): δ 7.52 (s, 1H), 4.50 (s, 1H), 3.95 (dt, J=13.5, 3.9 Hz, 2H), 3.79-3.62 (m, 1H), 3.18 (ddd, J=13.3, 11.6, 3.0 Hz, 2H), 2.10-2.02 (m, 2H), 1.58-1.48 (m, 2H), 1.46 (s, 9H).

### Methyl N-(2-(4-((tert-butoxycarbonyl)amino)piperidin-1-yl)thiazole-4-carbonyl)-O-(tert-butyldimethylsilyl)-L-serinate (12.3)

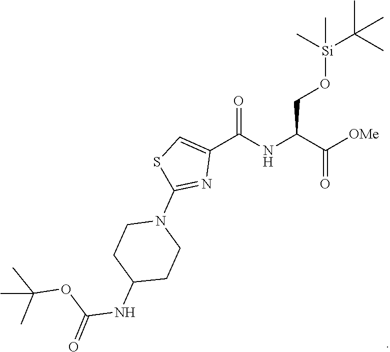 [SMILES: COC(=O)[C@H](CO[Si](C)(C)C(C)(C)C)NC(=O)c1csc(N2CCC(NC(=O)OC(C)(C)C)CC2)n1]

2-(4-((Tert-butoxycarbonyl)amino)piperidin-1-yl)thiazole-4-carboxylic acid (0.500 g, 1.53 mmole) was dissolved in DCM (3 mL). N,N′-Carbonyldiimidazole (0.262 g, 1.62 mmole) was added portionwise (gas evolution was observed) and the mixture was stirred at RT for 60 minutes. L-Serine methyl ester hydrochloride (0.262 g, 1.68 mmole) and N,N-diisopropylethylamine (0.320 mL, 1.84 mmole) were added and the solution was stirred at RT for 18 hours. Water (25 mL) was added and the mixture was extracted with DCM (3×10 mL). The combined organics were dried over magnesium sulfate, filtered, and concentrated. The residue was dissolved in DCM (5 mL). Imidazole (0.116 g, 1.70 mmole) and tert-butyldimethylchlorosilane (0.256 g, 1.70 mmole) were added and the mixture was stirred at RT for 90 minutes. Water (25 mL) was added and the mixture was extracted with DCM (3×10 mL). The combined organics were dried over magnesium sulfate, filtered, and concentrated to provide methyl N-(2-(4-((tert-butoxycarbonyl)amino)piperidin-1-yl)thiazole-4-carbonyl)-O-(tert-butyldimethylsilyl)-L-serinate as a pale yellow slurry (0.994 g). ¹H NMR (400 MHz, CDCl₃): δ 7.95 (d, J=8.8 Hz, 1H), 7.37 (s, 1H), 4.78 (ddd, J=8.8, 3.4, 2.5 Hz, 1H), 4.50 (s, 1H), 4.16 (dd, J=10.0, 2.6 Hz, 1H), 4.03-3.92 (m, 2H), 3.92-3.84 (m, 2H), 3.76 (s, 3H), 3.71 (d, J=15.5 Hz, 1H), 3.22-3.06 (m, 2H), 2.09-1.99 (m, 2H), 1.58-1.48 (m, 2H), 1.46 (s, 9H), 0.88 (s, 9H), 0.05 (s, 3H), 0.03 (s, 3H).

### N-(2-(4-((tert-butoxycarbonyl)amino)piperidin-1-yl)thiazole-4-carbonyl)-O-(tert-butyldimethylsilyl)-L-serine (12.4)

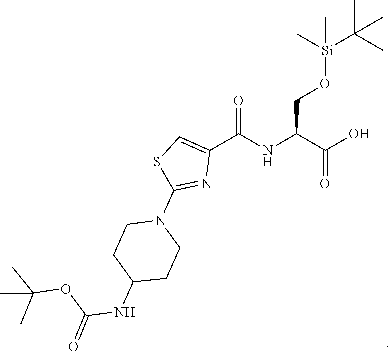 [SMILES: CC(C)(C)OC(=O)NC1CCN(c2nc(C(=O)N[C@@H](CO[Si](C)(C)C(C)(C)C)C(=O)O)cs2)CC1]

Using the procedure described in Example 4.3, methyl N-(2-(4-((tert-butoxycarbonyl)amino)piperidin-1-yl)thiazole-4-carbonyl)-O-(tert-butyldimethylsilyl)-L-serinate (0.994 g, 1.83 mmole) was converted to N-(2-(4-((tert-butoxycarbonyl)amino)piperidin-1-yl)thiazole-4-carbonyl)-O-(tert-butyldimethylsilyl)-L-serine as a yellow oil (0.900 g). ¹H NMR (400 MHz, CDCl₃): δ 7.97 (d, J=7.9 Hz, 1H), 7.41 (d, J=8.3 Hz, 1H), 4.75 (s, 1H), 4.54 (s, 1H), 4.22 (dd, J=10.0, 3.2 Hz, 1H), 3.92 (ddd, J=14.3, 8.6, 3.7 Hz, 3H), 3.80-3.63 (m, 1H), 3.22-3.06 (m, 2H), 2.08-1.99 (m, 2H), 1.42 (m, 11H), 0.90 (s, 9H), 0.08 (s, 6H).

### Methyl O-acetyl-N—(N-(2-(4-((tert-butoxycarbonyl)amino)piperidin-1-yl)thiazole-4-carbonyl)-O-(tert-butyldimethylsilyl)-L-seryl)-L-serinate (12.5)

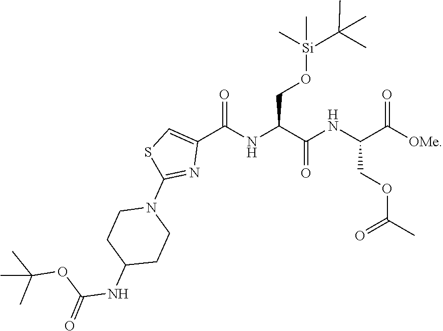 [SMILES: COC(=O)[C@H](COC(C)=O)NC(=O)[C@H](CO[Si](C)(C)C(C)(C)C)NC(=O)c1csc(N2CCC(NC(=O)OC(C)(C)C)CC2)n1]

N-(2-(4-((Tert-butoxycarbonyl)amino)piperidin-1-yl)thiazole-4-carbonyl)-O-(tert-butyldimethylsilyl)-L-serine (0.900 g, 1.70 mmole) was dissolved in DCM (4 mL) and N,N′-carbonyldiimidazole (0.306 g, 1.89 mmole) was added portionwise (gas evolution was observed). The solution was stirred at RT for 60 minutes and L-serine methyl ester hydrochloride (0.293 g, 1.88 mmole) and N,N-diisopropylethylamine (0.360 mL, 2.07 mmole) were added. The solution was stirred at RT for 2 hours and water (25 mL) was added. The mixture was extracted with DCM (3×10 mL). The combined organics were dried over magnesium sulfate, filtered, and concentrated. The residue was dissolved in DCM (4 mL) and triethylamine (0.270 mL, 1.93 mmole) and acetic anhydride (0.180 mL, 1.90 mmole) were added. The solution was stirred at RT for 18 hours and water (25 mL) was added. The mixture was extracted with DCM (3×10 mL). The combined organics were dried over magnesium sulfate, filtered, and concentrated. The crude residue was purified by silica gel chromatography to provide methyl O-acetyl-N—(N-(2-(4-((tert-butoxycarbonyl)amino)piperidin-1-yl)thiazole-4-carbonyl)-O-(tert-butyldimethylsilyl)-L-seryl)-L-serinate as a yellow oil (0.455 g, 40%). 1H NMR (400 MHz, CDCl₃): δ 7.99 (d, 1H), 7.41 (d, 1H), 7.39 (s, 1H), 4.84 (m, 1H), 4.55 (m, 1H), 4.49-4.20 (m, 2H), 4.28 (dd, 1H), 4.19 (dd, 1H), 4.00-3.87 (m, 2H), 3.75 (s, 3H), 3.74-3.59 (m, 2H), 3.21-3.07 (m, 2H), 2.03 (s, 3H), 1.52-1.47 (m, 2H), 1.45 (s, 9H), 0.91 (s, 9H), 0.12 (s, 3H), 0.11 (s, 3H).

### Methyl (S)-2-(2-(2-(4-((tert-butoxycarbonyl)amino)piperidin-1-yl)thiazole-4-carboxamido)-3-hydroxypropanamido)acrylate (12.6)

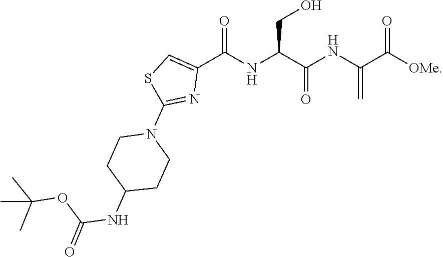 [SMILES: C=C(NC(=O)[C@H](CO)NC(=O)c1csc(N2CCC(NC(=O)OC(C)(C)C)CC2)n1)C(=O)OC]

Methyl O-acetyl-N—(N-(2-(4-((tert-butoxycarbonyl)amino)piperidin-1-yl)thiazole-4-carbonyl)-O-(tert-butyldimethylsilyl)-L-seryl)-L-serinate (0.455 g, 0.677 mmole) was dissolved in THE (1.5 mL). Tetrabutylammonium fluoride (1M solution in THF, 2.00 mL, 2.00 mmole) was added and the solution was stirred at RT for 5 hours. Water (25 mL) was added and the mixture was extracted with ethyl acetate (3×10 mL). The combined organics were dried over magnesium sulfate, filtered, and concentrated to provide methyl (S)-2-(2-(2-(4-((tert-butoxycarbonyl)amino)piperidin-1-yl)thiazole-4-carboxamido)-3-hydroxypropanamido)acrylate as a thick, yellow oil (0.347 g). ¹H NMR (400 MHz, CDCl₃): δ 9.07 (s, 1H), 8.07 (d, J=7.6 Hz, 1H), 7.43 (d, J=11.8 Hz, 1H), 6.56 (s, 1H), 5.94 (d, J=1.4 Hz, 1H), 4.70 (ddd, J=7.8, 5.1, 3.2 Hz, 1H), 4.53 (s, 1H), 4.26 (dd, J=11.5, 3.2 Hz, 1H), 4.02-3.90 (m, 3H), 3.83 (s, 3H), 3.81-3.73 (m, 1H), 3.67 (d, J=19.7 Hz, 1H), 3.21-3.07 (m, 2H), 2.07 (d, J=16.5 Hz, 2H), 1.51-1.47 (m, 2H), 1.46 (s, 9H).

### Methyl 2-(2-(2-(4-((tert-butoxycarbonyl)amino)piperidin-1-yl)thiazole-4-carboxamido)acrylamido)acrylate (12)

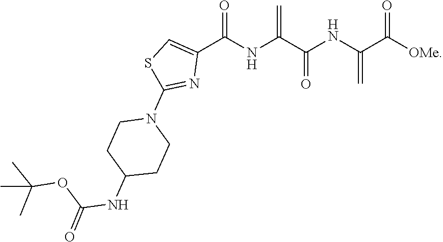 [SMILES: C=C(NC(=O)c1csc(N2CCC(NC(=O)OC(C)(C)C)CC2)n1)C(=O)NC(=C)C(=O)OC]

Methyl (S)-2-(2-(2-(4-((tert-butoxycarbonyl)amino)piperidin-1-yl)thiazole-4-carboxamido)-3-hydroxypropanamido)acrylate (0.347 g, 0.697 mmole) was dissolved in DCM (1.5 mL) and cooled to 0° C. Triethylamine (0.150 mL, 1.07 mmole) was added followed by methanesulfonyl chloride (0.080 mL, 1.03 mmole) and the mixture was stirred at 0° for 90 minutes. Water (25 mL) was added and the mixture was extracted with DCM (3×10 mL). The combined organics were dried over magnesium sulfate, filtered, and concentrated. The residue was dissolved in THE (1.5 mL) and cooled to 0° C. 1,8-Diazabicyclo[5.4.0]undec-7-ene (0.160 mL, 1.07 mmole) was added and the solution was stirred at 0° for 90 minutes. Water (25 mL) was added and the mixture was extracted with ethyl acetate (3×10 mLO. The combined organics were dried over magnesium sulfate, filtered, and concentrated. The crude residue was purified by silica gel chromatography to provide methyl 2-(2-(2-(4-((tert-butoxycarbonyl)amino)piperidin-1-yl)thiazole-4-carboxamido)acrylamido)acrylate as a white solid (0.124 g, 38% from 27.5). ¹H NMR (400 MHz, CDCl₃): δ 9.71 (s, 1H), 8.51 (s, 1H), 7.42 (s, 1H), 6.71 (d, J=2.1 Hz, 1H), 6.66 (s, 1H), 6.00 (d, J=1.3 Hz, 1H), 5.43 (t, J=1.9 Hz, 1H), 4.49 (s, 1H), 4.03-3.93 (m, 2H), 3.89 (s, 3H), 3.70 (s, 1H), 3.23-3.10 (m, 2H), 2.12-2.04 (m, 2H), 1.57-1.48 (m, 2H), 1.46 (s, 9H).

### Compound 13: Methyl 2-(2-(2-(4-(6-((tert-butoxycarbonyl)amino)hexanamido)piperidin-1-yl)thiazole-4-carboxamido)acrylamido)acrylate

### Ethyl 2-(4-(6-((tert-butoxycarbonyl)amino)hexanamido)piperidin-1-yl)thiazole-4-carboxylate (13.1)

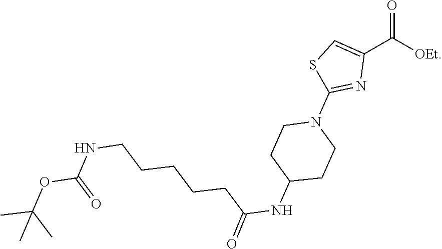 [SMILES: CCOC(=O)c1csc(N2CCC(NC(=O)CCCCCNC(=O)OC(C)(C)C)CC2)n1]

Ethyl 2-(4-((tert-butoxycarbonyl)amino)piperidin-1-yl)thiazole-4-carboxylate (Example 12.1, 7.176 g, 20.2 mmole) was dissolved in DCM (40 mL). HCl (4M solution in 1,4-dioxane, 20.0 mL, 80.0 mmole) was added and the mixture was stirred at RT for 60 minutes, then was concentrated. In a separate flask, 6-((tert-butoxycarbonyl)amino)hexanoic acid (5.141 g, 22.2 mmole) was dissolved in DCM (40 mL). N,N′-Carbonyldiimidazole (3.765 g, 23.2 mmole) was added portionwise (gas evolution was observed) and the solution was stirred at RT for 90 minutes. The solution was added to the amine hydrochloride generated above and N,N-diisopropylethylamine (4.20 mL, 24.1 mmole) was added. The resulting solution was stirred at RT for 18 hours. Water (100 mL) was added and the two layers were separated. The aqueous layer was extracted with DCM (2×20 mL). The combined organics were dried over sodium sulfate, filtered, and concentrated. The crude residue was purified by silica gel chromatography to provide ethyl 2-(4-(6-((tert-butoxycarbonyl)amino)hexanamido)piperidin-1-yl)thiazole-4-carboxylate as a pale yellow solid (8.120 g, 86%). ¹H NMR (400 MHz, CDCl₃): δ 7.43 (d, J=1.5 Hz, 1H), 5.66 (d, J=7.9 Hz, 1H), 4.60 (s, 1H), 4.35 (q, J=7.1 Hz, 2H), 4.06-3.94 (m, 3H), 3.21-3.13 (m, 2H), 3.13-3.04 (m, 3H), 2.17 (t, J=7.6 Hz, 2H), 2.06-1.99 (m, 2H), 1.65 (p, J =7.5 Hz, 2H), 1.54-1.47 (m, 3H), 1.44 (s, 9H), 1.40-1.29 (m, 5H).

### 2-(4-(6-((Tert-butoxycarbonyl)amino)hexanamido)piperidin-1-yl)thiazole-4-carboxylic acid (13.2)

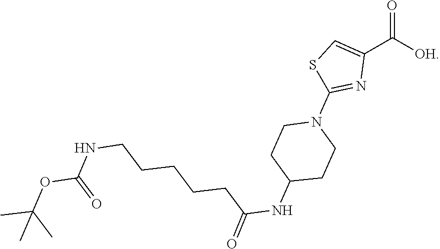 [SMILES: CC(C)(C)OC(=O)NCCCCCC(=O)NC1CCN(c2nc(C(=O)O)cs2)CC1]

Using the procedure described in Example 1.3, ethyl 2-(4-(6-((tert-butoxycarbonyl)amino)hexanamido)piperidin-1-yl)thiazole-4-carboxylate (8.120 g, 17.3 mmole) was converted to 2-(4-(6-((tert-butoxycarbonyl)amino)hexanamido)piperidin-1-yl)thiazole-4-carboxylic acid as a golden oil (9.950 g) that was carried forward without additional purification.

### Methyl N-(2-(4-(6-((tert-butoxycarbonyl)amino)hexanamido)piperidin-1-yl)thiazole-4-carbonyl)-O-(tert-butyldimethylsilyl)-L-serinate (13.3)

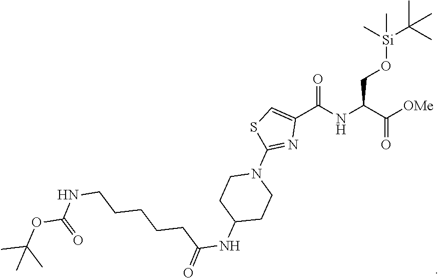 [SMILES: COC(=O)[C@H](CO[Si](C)(C)C(C)(C)C)NC(=O)c1csc(N2CCC(NC(=O)CCCCCNC(=O)OC(C)(C)C)CC2)n1]

Using the procedure described in Example 12.3,2-(4-(6-((tert-butoxycarbonyl)amino)hexanamido)piperidin-1-yl)thiazole-4-carboxylic acid (9.950 g, 22.6 mmole) was converted to methyl N-(2-(4-(6-((tert-butoxycarbonyl)amino)hexanamido)piperidin-1-yl)thiazole-4-carbonyl)-O-(tert-butyldimethylsilyl)-L-serinate as a yellow oil (6.704 g, 59% from 29.1). ¹H NMR (400 MHz, CDCl₃): δ 7.95 (d, J=8.8 Hz, 1H), 7.38 (s, 1H), 5.60-5.49 (m, 1H), 4.78 (ddd, J=8.8, 3.3, 2.5 Hz, 1H), 4.58 (s, 1H), 4.20-4.15 (m, 1H), 4.03 (tdd, J=11.4, 7.8, 4.0 Hz, 2H), 3.97-3.90 (m, 1H), 3.88 (dd, J=10.0, 3.4 Hz, 1H), 3.76 (s, 3H), 3.21-3.13 (m, 2H), 3.11 (dq, J=7.7, 5.5, 3.7 Hz, 2H), 2.17 (t, J=7.6 Hz, 2H), 2.05 (m, 2H), 1.73-1.60 (m, 2H), 1.57-1.46 (m, 4H), 1.44 (s, 9H), 1.40-1.31 (m, 2H), 0.88 (s, 9H), 0.05 (s, 3H), 0.02 (s, 3H).

### Methyl N-(2-(4-(6-((tert-butoxycarbonyl)amino)hexanamido)piperidin-1-yl)thiazole-4-carbonyl)-O-(tert-butyldimethylsilyl)-L-seryl-L-serinate (13.4)

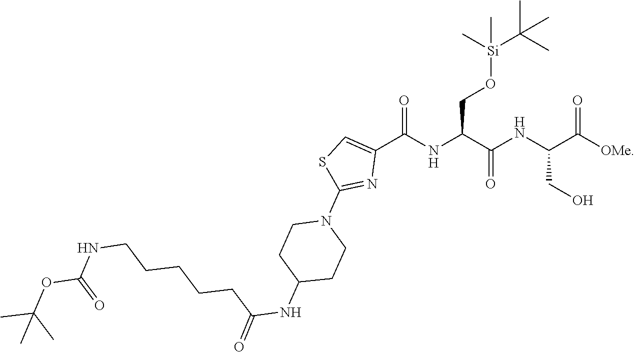 [SMILES: COC(=O)[C@H](CO)NC(=O)[C@H](CO[Si](C)(C)C(C)(C)C)NC(=O)c1csc(N2CCC(NC(=O)CCCCCNC(=O)OC(C)(C)C)CC2)n1]

Methyl N-(2-(4-(6-((tert-butoxycarbonyl)amino)hexanamido)piperidin-1-yl)thiazole-4-carbonyl)-O-(tert-butyldimethylsilyl)-L-serinate (6.704 g, 10.2 mmole) was dissolved in 4/1/1 THF/methanol/water (30 mL). Lithium hydroxide (0.738 g, 30.8 mmole) was added and the mixture was stirred at RT for 2 hours. Water (100 mL) was added and the solution was treated with 1N aq. HCl to pH=4. The mixture was extracted with diethyl ether (4×25 mL) and ethyl acetate (1×25 mL). The combined organics were dried over magnesium sulfate, filtered, and concentrated to provide N-(2-(4-(6-((tert-butoxycarbonyl)amino)hexanamido)piperidin-1-yl)thiazole-4-carbonyl)-O-(tert-butyldimethylsilyl)-L-serine.

N-(2-(4-(6-((Tert-butoxycarbonyl)amino)hexanamido)piperidin-1-yl)thiazole-4-carbonyl)-O-(tert-butyldimethylsilyl)-L-serine (0.505 g, 0.787 mmole) was dissolved in DCM (1.6 mL). L-Serine methyl ester hydrochloride (0.292 g, 1.88 mmole) was added followed by N,N-diisopropylethylamine (0.270 mL, 1.55 mmole) and pyBOP (0.490 g, 0.942 mmole). The mixture was stirred at RT for 4 hours and water (25 mL) was added. The mixture was extracted with DCM (3×10 mL). The combined organics were dried over magnesium sulfate, filtered, and concentrated. The crude residue was purified by silica gel chromatography to provide methyl N-(2-(4-(6-((tert-butoxycarbonyl)amino)hexanamido)piperidin-1-yl)thiazole-4-carbonyl)-O-(tert-butyldimethylsilyl)-L-seryl-L-serinate as a thick, colorless gel (0.401 g, 69%). 1H NMR (400 MHz, CDCl₃): δ 9.07 (s, 1H), 8.05 (d, J=8.8 Hz, 1H), 7.48 (s, 1H), 6.54 (s, 1H), 5.96 (s, 1H), 5.53-5.47 (m, 1H), 4.72-4.69 (m, 1H), 4.63-4.57 (m, 2H), 4.29-4.22 (m, 1H), 4.09-3.92 (m, 4H), 3.81 (s, 3H), 3.79-3.75 (m, 1H), 3.22-3.17 (m, 2H), 3.16-3.05 (m, 2H), 2.20 (t, J=7.5 Hz, 2H), 2.05-1.97 (m, 2H), 1.70-1.59 (m, 2H), 1.51-1.40 (m, 4H), 1.43 (s, 9H), 1.38-1.27 (m, 2H).

### Methyl (S)-2-(2-(2-(4-(6-((tert-butoxycarbonyl)amino)hexanamido)piperidin-1-yl)thiazole-4-carboxamido)-3-hydroxypropanamido)acrylate (13)

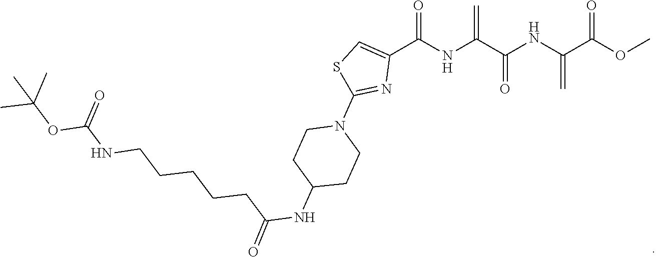 [SMILES: C=C(NC(=O)c1csc(N2CCC(NC(=O)CCCCCNC(=O)OC(C)(C)C)CC2)n1)C(=O)NC(=C)C(=O)OC]

Methyl N-(2-(4-(6-((tert-butoxycarbonyl)amino)hexanamido)piperidin-1-yl)thiazole-4-carbonyl)-O-(tert-butyldimethylsilyl)-L-seryl-L-serinate (0.401 g, 0.540 mmole) was dissolved in DCM (2 mL). Triethylamine (0.084 mL, 0.599 mmole) and acetic anhydride (0.060 mL, 0.635 mmole) were added and the mixture was stirred at RT for 18 hours. Water (25 mL) was added and the mixture was extracted with DCM (3×10 mL). The combined organics were dried over magnesium sulfate, filtered, and concentrated. The residue was dissolved in THE and tetrabutylammonium fluoride (1M solution in THF, 1.60 mL, 1.60 mmole) was added. The solution was stirred at RT for 2 hours and water (25 mL) was added. The mixture was extracted with ethyl acetate (3×10 mL). The combined organics were dried over magnesium sulfate, filtered, and concentrated to provide methyl (S)-2-(2-(2-(4-(6-((tert-butoxycarbonyl)amino)hexanamido)piperidin-1-yl)thiazole-4-carboxamido)-3-hydroxypropanamido)acrylate as a white solid (0.318 g, 96%).

Methyl (S)-2-(2-(2-(4-(6-((tert-butoxycarbonyl)amino)hexanamido)piperidin-1-yl)thiazole-4-carboxamido)-3-hydroxypropanamido)acrylate (0.318 g, 0.521 mmole) was dissolved in DCM (2 mL) and cooled to 0° C. Triethylamine (0.110 mL, 0.785 mmole) was added followed by methanesulfonyl chloride (0.060 mL, 0.775 mmole). The mixture was stirred at 0° for 2 hours and water (25 mL) was added. The mixture was extracted with DCM (3×10 mL). The combined organics were dried over magnesium sulfate, filtered, and concentrated. The residue was dissolved in THE (2 mL) and cooled to 0° C. 1,8-Diazabicyclo[5.4.0]undec-7-ene (0.120 mL, 0.802 mmole) was added and the solution was stirred at 0° for 2 hours. Water (25 mL) was added and the mixture was extracted with ethyl acetate (3×10 mL). The combined organics were dried over magnesium sulfate, filtered, and concentrated. The crude residue was purified by silica gel chromatography to provide methyl 2-(2-(2-(4-(6-((tert-butoxycarbonyl)amino)hexanamido)piperidin-1-yl)thiazole-4-carboxamido)acrylamido)acrylate as a white solid (0.106 g, 34%). ¹H NMR (400 MHz, CDCl₃): δ 9.71 (s, 1H), 8.51 (s, 1H), 7.43 (s, 1H), 6.72 (d, J=2.1 Hz, 1H), 6.66 (s, 1H), 6.00 (d, J=1.3 Hz, 1H), 5.47-5.36 (m, 2H), 4.54 (s, 1H), 4.07-3.95 (m, 3H), 3.89 (s, 3H), 3.22-3.05 (m, 4H), 2.17 (t, J=7.5 Hz, 2H), 2.10-2.00 (m, 2H), 1.66 (p, J=7.4 Hz, 2H), 1.51 (h, J=7.5 Hz, 4H), 1.43 (s, 9H), 1.40-1.30 (m, 2H).

### Compound 14: Tert-butyl 3-(4-((3-((3-methoxy-3-oxoprop-1-en-2-yl)amino)-3-oxoprop-1-en-2-yl)carbamoyl)thiazol-2-yl)benzoate

### Tert-butyl 3-carbamothioylbenzoate (14.1)

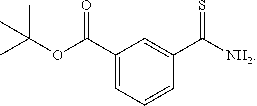 [SMILES: CC(C)(C)OC(=O)c1cccc(C(N)=S)c1]

Tert-butyl 3-cyanobenzoate, prepared as described in Adv. Synth. Catal., 2014, 356(14-15), 3074-3082, (11.887 g, 58.5 mmole) was dissolved in pyridine (60 mL). Triethylamine (9.00 mL, 64.2 mmole) was added followed by ammonium sulfide (40% aqueous solution, 12.0 mL, 70.2 mmole) and the mixture was heated to 50° C. for 4 hours. The mixture was cooled to RT and concentrated. The residue was dissolved in ethyl acetate (75 mL) and washed sequentially with 0.5N aq. HCl (3×50 mL), sat. aq. sodium bicarbonate (1×25 mL), and sat. aq. sodium chloride (1×25 mL). The organics were dried over magnesium sulfate, filtered, and concentrated to provide tert-butyl 3-carbamothioylbenzoate as a yellow solid (11.043 g, 68%). ¹H NMR (400 MHz, DMSO-d₆): δ 10.02 (s, 1H), 9.68 (s, 1H), 8.41 (td, J=1.9, 0.5 Hz, 1H), 8.07 (ddd, J=7.8, 2.0, 1.2 Hz, 1H), 8.01 (ddd, J=7.8, 1.8, 1.2 Hz, 1H), 7.55 (td, J=7.8, 0.5 Hz, 1H), 1.57 (s, 9H).

### Ethyl 2-(3-(tert-butoxycarbonyl)phenyl)thiazole-4-carboxylate (14.2)

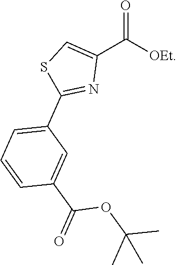 [SMILES: CCOC(=O)c1csc(-c2cccc(C(=O)OC(C)(C)C)c2)n1]

Tert-butyl 3-carbamothioylbenzoate (11.043 g, 46.5 mmole) was dissolved in 1,4-dioxane (235 mL) and cooled to 0° C. Potassium bicarbonate (37.398 g, 374 mmole) was added followed by ethyl bromopyruvate (11.7 mL, 93.2 mmole). The mixture was stirred vigorously at 0° for 2 hours, then at RT for 18 hours. The mixture was concentrated and the residue was dissolved in water (200 mL) and ethyl acetate (100 mL). The two layers were separated and the aqueous layer was extracted with ethyl acetate (2×100 mL). The combined organics were washed with sat. aq. sodium chloride (1×100 mL), dried over magnesium sulfate, filtered, and concentrated. The residue was dissolved in 1,4-dioxane (235 mL) and cooled to 0° C. Pyridine (30.0 mL, 371 mmole) was added followed by slow addition of trifluoroacetic anhydride (19.5 mL, 140 mmole) and the solution was stirred at 0° for 90 minutes, then at RT for 2 hours. The mixture was concentrated and the residue was dissolved in ethyl acetate (300 mL). The solution was washed sequentially with 1N aq. HCl (3×50 mL), sat. aq. sodium bicarbonate (1×50 mL), and sat. aq. sodium chloride (1×50 mL). The organics were dried over magnesium sulfate, filtered, and concentrated. The crude residue was split into two portions and each was purified by silica gel chromatography. The fractions containing the desired product were combined and concentrated to provide ethyl 2-(3-(tert-butoxycarbonyl)phenyl)thiazole-4-carboxylate as an orange oil (17.31 g). ¹H NMR (400 MHz, CDCl₃): δ 8.55-8.48 (m, 1H), 8.23 (ddd, J=7.8, 1.9, 1.2 Hz, 1H), 8.20 (s, 1H), 8.08 (dt, J=7.8, 1.4 Hz, 1H), 7.53 (td, J=7.8, 0.6 Hz, 1H), 4.46 (q, J =7.1 Hz, 2H), 1.63 (s, 9H), 1.44 (d, J=14.3 Hz, 3H).

### 2-(3-(tert-butoxycarbonyl)phenyl)thiazole-4-carboxylic acid (14.3)

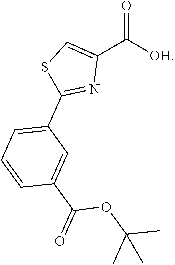 [SMILES: CC(C)(C)OC(=O)c1cccc(-c2nc(C(=O)O)cs2)c1]

Ethyl 2-(3-(tert-butoxycarbonyl)phenyl)thiazole-4-carboxylate (17.31 g, 51.9 mmole) was dissolved in 4/1/1 THF/methanol/water (200 mL). Lithium hydroxide (3.733 g, 156 mmole) was added and the mixture was stirred vigorously at RT for 2 hours. The mixture was filtered and the filtrate was stirred with additional lithium hydroxide (−0.5 g) for 60 minutes, then was concentrated to the aqueous layer. Water (200 mL) was added and the mixture was combined with the solid above. The mixture was treated with 6N aq. HCl to pH=4. The solid was collected by filtration and washed with water, then allowed to air dry to provide 2-(3-(tert-butoxycarbonyl)phenyl)thiazole-4-carboxylic acid as a white solid (16.847 g). ¹H NMR (400 MHz, DMSO-d₆): δ 8.42 (t, J=1.7 Hz, 1H), 8.21 (ddd, J=7.8, 1.9, 1.1 Hz, 1H), 7.99 (dt, J=7.8, 1.3 Hz, 1H), 7.94 (s, 1H), 7.64 (t, J=7.8 Hz, 1H), 1.59 (s, 9H)

### Tert-butyl (S)-3-(4-((3-((tert-butyldimethylsilyl)oxy)-1-methoxy-1-oxopropan-2-yl)carbamoyl)thiazol-2-yl)benzoate (14.4)

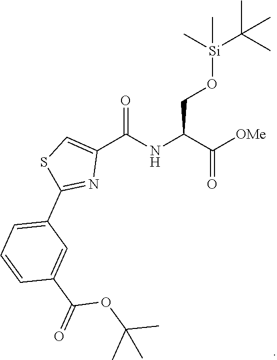 [SMILES: COC(=O)[C@H](CO[Si](C)(C)C(C)(C)C)NC(=O)c1csc(-c2cccc(C(=O)OC(C)(C)C)c2)n1]

2-(3-(Tert-butoxycarbonyl)phenyl)thiazole-4-carboxylic acid (16.847 g, 55.2 mmole) was suspended in DMF (220 mL). L-Serine methyl ester hydrochloride (10.357 g, 66.6 mmole) was added followed by 1-hydroxybenzotriazole hydrate (9.312 g, 60.8 mmole), 1-(3-dimethylaminopropyl)-3-ethylcarbodiimide hydrochloride (11.664 g, 60.8 mmole), and N,N-diisopropylethylamine (29.0 mL, 166 mmole). The mixture was stirred at RT for 18 hours and water (300 mL) and 1N aq. HCl (200 mL) were added. The mixture was extracted with ethyl acetate (4×100 mL). The combined organics were washed with sat. aq. sodium chloride (1×100 mL), dried over magnesium sulfate, filtered, and concentrated. The residue was dissolved in DCM (110 mL) and imidazole (4.160 g, 61.1 mmole) and tert-butyldimethylchlorosilane (9.158 g, 60.8 mmole) were added. The mixture was stirred at RT for 90 minutes and water (300 mL) was added. The two layers were separated and the aqueous layer was extracted with DCM (2×50 mL). The combined organics were dried over magnesium sulfate, filtered, and concentrated. The crude residue was purified by silica gel chromatography to provide tert-butyl (S)-3-(4-((3-((tert-butyldimethylsilyl)oxy)-1-methoxy-1-oxopropan-2-yl)carbamoyl)thiazol-2-yl)benzoate as a white solid (15.343 g, 63% from Intermediate 30.2). ¹H NMR (400 MHz, CDCl₃): δ 8.52-8.44 (m, 1H), 8.21 (ddd, J=7.8, 1.9, 1.2 Hz, 2H), 8.14 (s, 1H), 8.07 (dt, J=7.8, 1.4 Hz, 1H), 7.52 (td, J=7.8, 0.6 Hz, 1H), 4.87 (dt, J=8.7, 3.1 Hz, 1H), 4.21 (dd, J=10.1, 2.8 Hz, 1H), 3.96 (dd, J=10.0, 3.4 Hz, 1H), 3.79 (s, 3H), 1.64 (s, 9H), 0.91 (s, 9H), 0.08 (s, 3H), 0.06 (s, 3H).

### N-(2-(3-(tert-butoxycarbonyl)phenyl)thiazole-4-carbonyl)-O-(tert-butyldimethylsilyl)-L-serine (14.5)

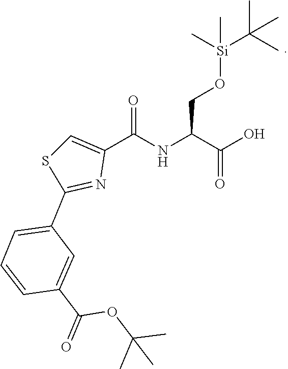 [SMILES: CC(C)(C)OC(=O)c1cccc(-c2nc(C(=O)N[C@@H](CO[Si](C)(C)C(C)(C)C)C(=O)O)cs2)c1]

Tert-butyl (S)-3-(4-((3-((tert-butyldimethylsilyl)oxy)-1-methoxy-1-oxopropan-2-yl)carbamoyl)thiazol-2-yl)benzoate (15.343 g, 29.5 mmole) was dissolved in 4/1/1 THF/methanol/water (90 mL). Lithium hydroxide (1.425 g, 59.5 mmole) was added and the mixture was stirred at RT for 3 hours. Water (300 mL) was added and the mixture was treated with 1N aq. HCl to pH=4. The mixture was extracted with ethyl acetate (4×75 mL). The combined organics were dried over magnesium sulfate, filtered, and concentrated to provide N-(2-(3-(tert-butoxycarbonyl)phenyl)thiazole-4-carbonyl)-O-(tert-butyldimethylsilyl)-L-serine as a white solid (14.560 g, 97%). ¹H NMR (400 MHz, DMSO-d₆): δ 8.46 (d, J=9.3 Hz, 2H), 8.25-8.15 (m, 2H), 8.03 (dt, J=7.8, 1.3 Hz, 1H), 7.69 (dt, J=9.7, 7.7 Hz, 1H), 4.60 (dt, J=8.4, 3.5 Hz, 1H), 4.11 (dd, J=10.3, 3.5 Hz, 1H), 3.97 (dd, J=10.4, 3.6 Hz, 1H), 1.58 (s, 9H), 0.85 (s, 9H), 0.05 (s, 3H), 0.03 (s, 3H)

### Tert-butyl 3-(4-(((4S,7S)-4-(hydroxymethyl)-10,10,11,11-tetramethyl-3,6-dioxo-2,9-dioxa-5-aza-10-siladodecan-7-yl)carbamoyl)thiazol-2-yl)benzoate (14.6)

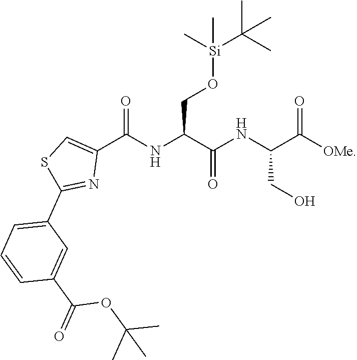 [SMILES: COC(=O)[C@H](CO)NC(=O)[C@H](CO[Si](C)(C)C(C)(C)C)NC(=O)c1csc(-c2cccc(C(=O)OC(C)(C)C)c2)n1]

N-(2-(3-(Tert-butoxycarbonyl)phenyl)thiazole-4-carbonyl)-O-(tert-butyldimethylsilyl)-L-serine (3.142 g, 6.20 mmole) was dissolved in DCM (12 mL). L-Serine methyl ester hydrochloride (1.110 g, 7.13 mmole) was added followed by N,N-diiisopropylethylamine (2.10 mL, 12.1 mmole), HOBt·H₂O (1.099 g, 7.18 mmole) and EDC·HCl (1.371 g, 7.15 mmole). The mixture was stirred at RT for 18 hours. Water (50 mL) was added and the two layers were separated. The aqueous layer was extracted with DCM (2×15 mL). The combined organics were dried over magnesium sulfate, filtered, and concentrated. The crude residue was purified by silica gel chromatography to provide tert-butyl 3-(4-(((4S,7S)-4-(hydroxymethyl)-10,10,11,11-tetramethyl-3,6-dioxo-2,9-dioxa-5-aza-10-siladodecan-7-yl)carbamoyl)thiazol-2-yl)benzoate as a white solid (2.159 g, 57%). ¹H NMR (400 MHz, CDCl₃): δ 8.49-8.41 (m, 1H), 8.28-8.21 (m, 1H), 8.21-8.16 (m, 1H), 8.14 (s, 1H), 8.06 (dq, J=7.8, 1.7 Hz, 1H), 7.56-7.45 (m, 2H), 4.68 (tdd, J=11.3, 8.0, 4.0 Hz, 2H), 4.20 (dt, J=9.8, 4.3 Hz, 1H), 4.05-3.94 (m, 2H), 3.90-3.82 (m, 1H), 3.78 (d, J=7.9 Hz, 3H), 1.63 (d, J=1.0 Hz, 9H), 0.94 (d, J=1.2 Hz, 9H), 0.14 (d, J=1.2 Hz, 3H), 0.14 (s, 3H), 0.13 (s, 3H)

### Tert-butyl 3-(4-(((S)-3-hydroxy-1-(((S)-3-hydroxy-1-methoxy-1-oxopropan-2-yl)amino)-1-oxopropan-2-yl)carbamoyl)thiazol-2-yl)benzoate (14.7)

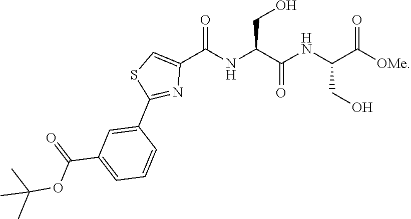 [SMILES: COC(=O)[C@H](CO)NC(=O)[C@H](CO)NC(=O)c1csc(-c2cccc(C(=O)OC(C)(C)C)c2)n1]

Tert-butyl 3-(4-(((4S,7S)-4-(hydroxymethyl)-10,10,11,11-tetramethyl-3,6-dioxo-2,9-dioxa-5-aza-10-siladodecan-7-yl)carbamoyl)thiazol-2-yl)benzoate (2.159 g, 3.55 mmole) was dissolved in THE (7 mL). Tetrabutylammonium fluoride (1M solution in THF, 3.90 mL, 3.90 mmole) was added and the solution was stirred at RT for 2 ½ hours. Water (50 mL) was added and the mixture was extracted with ethyl acetate (3×15 mL). The combined organics were dried over magnesium sulfate, filtered, and concentrated. The crude residue was purified by silica gel chromatography to provide tert-butyl 3-(4-(((S)-3-hydroxy-1-(((S)-3-hydroxy-1-methoxy-1-oxopropan-2-yl)amino)-1-oxopropan-2-yl)carbamoyl)thiazol-2-yl)benzoate as a white solid (1.346 g, 77%). ¹H NMR (400 MHz, CDCl₃): δ 8.44-8.36 (m, 2H), 8.17-8.09 (m, 2H), 8.03 (dq, J=7.8, 1.2 Hz, 1H), 7.90 (d, J=7.9 Hz, 1H), 7.49 (t, J=7.7 Hz, 1H), 4.92-4.80 (m, 1H), 4.75 (dq, J=7.5, 3.6 Hz, 1H), 4.17-4.10 (m, 1H), 4.05 (ddd, J=11.6, 7.4, 3.5 Hz, 2H), 3.93 (td, J=11.3, 10.1, 6.8 Hz, 1H), 3.81 (s, 3H), 1.63 (s, 9H).

### Tert-butyl 3-(4-((3-((3-methoxy-3-oxoprop-1-en-2-yl)amino)-3-oxoprop-1-en-2-yl)carbamoyl)thiazol-2-yl)benzoate (14)

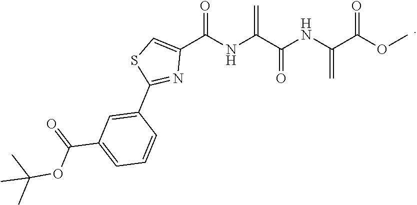 [SMILES: C=C(NC(=O)c1csc(-c2cccc(C(=O)OC(C)(C)C)c2)n1)C(=O)NC(=C)C(=O)OC]

Tert-butyl 3-(4-(((S)-3-hydroxy-1-(((S)-3-hydroxy-1-methoxy-1-oxopropan-2-yl)amino)-1-oxopropan-2-yl)carbamoyl)thiazol-2-yl)benzoate (0.200 g, 0.405 mmole) was dissolved in DCM (1 mL) and cooled to 0° C. Triethylamine (0.285 mL, 2.03 mmole) was added followed by methanesulfonyl chloride (0.160 mL, 2.07 mmole). The mixture was stirred at 0° for 60 minutes. Water (25 mL) was added and the mixture was extracted with DCM (3×10 mL). The combined organics were dried over magnesium sulfate, filtered, and concentrated. The residue was dissolved in THE and cooled to 0° C. 1,8-Diazabicyclo[5.4.0]undec-7-ene (0.300 mL, 2.01 mmole) was added and the solution was stirred at 0° for 90 minutes. Water (25 mL) was added and the mixture was extracted with ethyl acetate (3×10 mL). The combined organics were dried over magnesium sulfate, filtered, and concentrated. The crude residue was purified by silica gel chromatography to provide tert-butyl 3-(4-((3-((3-methoxy-3-oxoprop-1-en-2-yl)amino)-3-oxoprop-1-en-2-yl)carbamoyl)thiazol-2-yl)benzoate as a colorless oil (0.0338 g, 18%). ¹H NMR (400 MHz, CDCl₃): δ 10.06 (s, 1H), 8.56 (t, J=1.8 Hz, 2H), 8.24 (ddd, J=7.8, 1.9, 1.2 Hz, 1H), 8.18 (s, 1H), 8.09 (dt, J=7.8, 1.4 Hz, 1H), 7.55 (t, J=7.8 Hz, 1H), 6.78 (d, J=2.2 Hz, 1H), 6.71 (s, 1H), 6.02 (d, J=1.3 Hz, 1H), 5.51 (t, J=1.9 Hz, 1H), 3.91 (s, 3H), 1.65 (s, 9H).

### Compound 15: Methyl 2-(2-(2-(4-((2-((tert-butoxycarbonyl)amino)ethyl)carbamoyl)piperidin-1-yl)thiazole-4-carboxamido)acrylamido)acrylate

### Benzyl 4-((2-((tert-butoxycarbonyl)amino)ethyl)carbamoyl)piperidine-1-carboxylate (15.1)

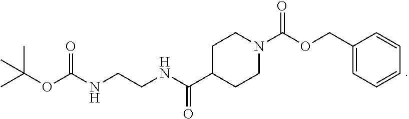 [SMILES: CC(C)(C)OC(=O)NCCNC(=O)C1CCN(C(=O)OCc2ccccc2)CC1]

1-((Benzyloxy)carbonyl)piperidine-4-carboxylic acid (2.006 g, 7.62 mmole) was dissolved in DCM (15 mL). N,N′-Carbonyldiimidazole (1.290 g, 7.96 mmole) was added portionwise (gas evolution was observed) and the solution was stirred at RT for 60 minutes. Tert-butyl (2-aminoethyl)carbamate (1.374 g, 8.58 mmole) was added slowly and the solution was stirred at RT for 2 hours. Water (50 mL) was added and the two layers were separated. The aqueous layer was extracted with DCM (2×15 mL). The combined organics were dried over magnesium sulfate, filtered, and concentrated. The crude residue was purified by silica gel chromatography to provide benzyl 4-((2-((tert-butoxycarbonyl)amino)ethyl)carbamoyl)piperidine-1-carboxylate as a white solid (2.984 g, 97%). ¹H NMR (400 MHz, CDCl₃): δ 7.41-7.28 (m, 5H), 6.43 (s, 1H), 5.12 (s, 2H), 4.92 (s, 1H), 4.20 (s, 2H), 3.40-3.23 (m, 4H), 2.83 (s, 2H), 2.24 (tt, J=11.5, 3.8 Hz, 1H), 1.84 (d, J=14.9 Hz, 2H), 1.64 (qd, J=11.7, 4.0 Hz, 2H), 1.43 (s, 9H)

### Tert-butyl (2-(piperidine-4-carboxamido)ethyl)carbamate (15.2)

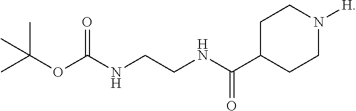

Palladium on carbon (10%, Degussa type, 0.818 g, 0.769 mmole) was added to benzyl 4-((2-((tert-butoxycarbonyl)amino)ethyl)carbamoyl)piperidine-1-carboxylate (2.984 g, 7.36 mmole). Ethanol (15 mL) was added dropwise on top of the palladium to wet the catalyst, then the remaining ethanol was slowly added. Ammonium formate (0.934 g, 14.8 mmole) was added and the mixture was heated to 70° C. for 4 hours, then was cooled to RT and diluted with DCM (30 mL). The mixture was filtered through a pad of Celite and the filter cake was washed with DCM. The combined filtrates were concentrated to provide tert-butyl (2-(piperidine-4-carboxamido)ethyl)carbamate as a brown solid (2.184 g). ¹H NMR (400 MHz, CDCl₃): δ 6.40 (s, 1H), 5.08 (s, 1H), 3.40-3.31 (m, 2H), 3.31-3.22 (m, 2H), 3.12 (dt, J=12.9, 3.7 Hz, 2H), 2.61 (td, J=12.3, 2.6 Hz, 2H), 2.22 (tt, J=11.8, 3.7 Hz, 1H), 1.87-1.77 (m, 2H), 1.65-1.51 (m, 2H), 1.40 (d, J=7.0 Hz, 9H).

### Ethyl 2-(4-((2-((tert-butoxycarbonyl)amino)ethyl)carbamoyl)piperidin-1-yl)thiazole-4-carboxylate (15.3)

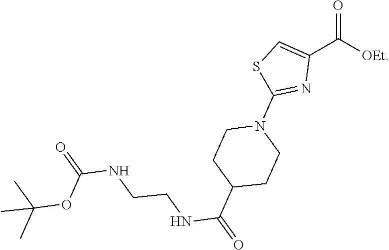 [SMILES: CCOC(=O)c1csc(N2CCC(C(=O)NCCNC(=O)OC(C)(C)C)CC2)n1]

Tert-butyl (2-(piperidine-4-carboxamido)ethyl)carbamate (2.184 g, 8.05 mmole) was dissolved in N,N-dimethylacetamide (16 mL). Ethyl 2-bromothiazole-4-carboxylate (1.913 g, 8.10 mmole) was added followed by triethylamine (1.25 mL, 8.92 mmole) and the mixture was heated to 80° C. for 24 hours. The mixture was cooled to RT and water (75 mL) was added. The mixture was extracted with ethyl acetate (3×20 mL) and the combined organics were dried over magnesium sulfate, filtered, and concentrated. The crude residue was purified by silica gel chromatography to provide ethyl 2-(4-((2-((tert-butoxycarbonyl)amino)ethyl)carbamoyl)piperidin-1-yl)thiazole-4-carboxylate as a pale yellow solid (2.174 g, 63%). ¹H NMR (400 MHz, CDCl₃): δ 7.44 (s, 1H), 6.57 (s, 1H), 4.96 (s, 1H), 4.35 (q, J=7.1 Hz, 2H), 4.08 (dt, J=13.3, 3.6 Hz, 2H), 3.41-3.32 (m, 2H), 3.29 (q, J=5.7 Hz, 2H), 3.11-3.02 (m, 2H), 2.31 (tt, J=11.5, 3.7 Hz, 1H), 2.02-1.90 (m, 2H), 1.89-1.77 (m, 2H), 1.44 (s, 9H), 1.37 (t, J=7.1 Hz, 3H).

### 2-(4-((2-((Tert-butoxycarbonyl)amino)ethyl)carbamoyl)piperidin-1-yl)thiazole-4-carboxylic acid (15.4)

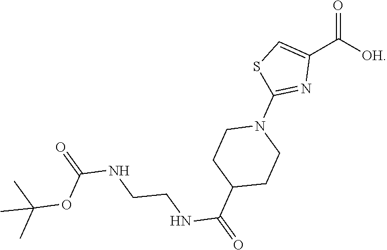 [SMILES: CC(C)(C)OC(=O)NCCNC(=O)C1CCN(c2nc(C(=O)O)cs2)CC1]

Ethyl 2-(4-((2-((tert-butoxycarbonyl)amino)ethyl)carbamoyl)piperidin-1-yl)thiazole-4-carboxylate (2.174 g, 5.10 mmole) was dissolved in 4/1/1 THF/methanol/water (15 mL). Lithium hydroxide (0.248 g, 10.4 mmole) was added and the mixture was stirred at RT for 2 hours. Water (75 mL) was added and the solution was treated with 1N aq. HCl to pH=4. The mixture was extracted with ethyl acetate (3×20 mL). A solid began to precipitate from the organics, which was collected by filtration and allowed to air dry to provide 2-(4-((2-((tert-butoxycarbonyl)amino)ethyl)carbamoyl)piperidin-1-yl)thiazole-4-carboxylic acid as a yellow solid. The filtrate was dried over magnesium sulfate, filtered, and concentrated to provide a second crop of 2-(4-((2-((tert-butoxycarbonyl)amino)ethyl)carbamoyl)piperidin-1-yl)thiazole-4-carboxylic acid. The two crops were combined (yellow solid, 1.346 g, 66%). ¹H NMR (400 MHz, DMSO-d₆): δ 12.58 (s, 1H), 7.85 (t, J=5.7 Hz, 1H), 7.61 (s, 1H), 6.79 (t, J=5.7 Hz, 1H), 3.89 (dt, J=12.9, 3.6 Hz, 2H), 3.06 (dt, J=12.2, 4.9 Hz, 4H), 3.01-2.88 (m, 2H), 2.34 (tt, J=11.3, 3.4 Hz, 1H), 1.87-1.71 (m, 2H), 1.67-1.49 (m, 2H), 1.37 (s, 9H).

### Methyl N-(2-(4-((2-((tert-butoxycarbonyl)amino)ethyl)carbamoyl)piperidin-1-yl)thiazole-4-carbonyl)-O-(tert-butyldimethylsilyl)-L-serinate (15.5)

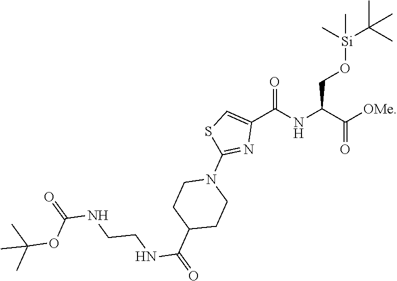 [SMILES: COC(=O)[C@H](CO[Si](C)(C)C(C)(C)C)NC(=O)c1csc(N2CCC(C(=O)NCCNC(=O)OC(C)(C)C)CC2)n1]

Using the procedure described in Example 12.3, 2-(4-((2-((tert-butoxycarbonyl)amino)ethyl)carbamoyl)piperidin-1-yl)thiazole-4-carboxylic acid (1.346 g, 3.38 mmole) was converted to methyl N-(2-(4-((2-((tert-butoxycarbonyl)amino)ethyl)carbamoyl)piperidin-1-yl)thiazole-4-carbonyl)-O-(tert-butyldimethylsilyl)-L-serinate as a thick, yellow oil (1.977 g, 95%). ¹H NMR (400 MHz, CDCl₃): δ 7.96 (d, J=8.8 Hz, 1H), 7.38 (s, 1H), 6.54 (s, 1H), 4.94 (s, 1H), 4.83-4.73 (m, 1H), 4.20-4.14 (m, 1H), 4.08-3.95 (m, 2H), 3.88 (dd, J=10.0, 3.4 Hz, 1H), 3.76 (s, 3H), 3.41-3.33 (m, 2H), 3.30 (q, J=5.8, 4.9 Hz, 2H), 3.11-2.97 (m, 2H), 2.31 (tt, J=11.5, 3.7 Hz, 1H), 1.95 (dd, J=13.4, 3.4 Hz, 2H), 1.89-1.75 (m, 2H), 1.45 (s, 9H), 0.88 (s, 9H), 0.05 (s, 3H), 0.03 (s, 3H).

### Methyl O-acetyl-N—(N-(2-(4-((2-((tert-butoxycarbonyl)amino)ethyl)carbamoyl)piperidin-1-yl)thiazole-4-carbonyl)-O-(tert-butyldimethylsilyl)-L-seryl)-L-serinate (15.6)

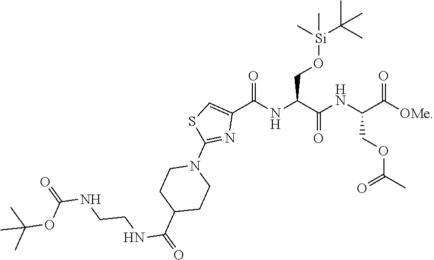 [SMILES: COC(=O)[C@H](COC(C)=O)NC(=O)[C@H](CO[Si](C)(C)C(C)(C)C)NC(=O)c1csc(N2CCC(C(=O)NCCNC(=O)OC(C)(C)C)CC2)n1]

Methyl N-(2-(4-((2-((tert-butoxycarbonyl)amino)ethyl)carbamoyl)piperidin-1-yl)thiazole-4-carbonyl)-O-(tert-butyldimethylsilyl)-L-serinate (1.977 g, 3.22 mmole) was dissolved in 4/1/1 THF/methanol/water (9 mL). Lithium hydroxide (0.157 g, 6.56 mmole) was added and the mixture was stirred at RT for 3 hours. Water (50 mL) was added and the solution was treated with 1N aq. HCl to pH=4. The mixture was extracted with ethyl acetate (3×15 mL). The combined organics were dried over magnesium sulfate, filtered, and concentrated to provide N-(2-(4-((2-((tert-butoxycarbonyl)amino)ethyl)carbamoyl)piperidin-1-yl)thiazole-4-carbonyl)-O-(tert-butyldimethylsilyl)-L-serine as a white solid (1.894 g, 98%).

N-(2-(4-((2-((Tert-butoxycarbonyl)amino)ethyl)carbamoyl)piperidin-1-yl)thiazole-4-carbonyl)-O-(tert-butyldimethylsilyl)-L-serine (0.408 g, 0.680 mmole) was suspended in DCM (1.4 mL). L-Serine methyl ester hydrochloride (0.131 g, 0.842 mmole) was added followed by N,N-diisopropylethylamine (0.350 mL, 2.01 mmole), HOBt·H₂O (0.131 g, 0.855 mmole) and EDC·HCl (0.155 g, 0.809 mmole). The mixture was stirred at RT for 18 hours and water (25 mL) was added. The mixture was extracted with DCM (3×10 mL). The combined organics were dried over magnesium sulfate, filtered, and concentrated. The residue was dissolved in DCM (1.4 mL). Triethylamine (0.115 mL, 0.821 mmole) and acetic anhydride (0.076 mL, 0.804 mmole) were added and the solution was stirred at RT for 2 hours. Water (25 mL) was added and the mixture was extracted with DCM (3×10 mL). The combined organics were dried over magnesium sulfate, filtered, and concentrated. The crude residue was purified by silica gel chromatography to provide methyl O-acetyl-N—(N-(2-(4-((2-((tert-butoxycarbonyl)amino)ethyl)carbamoyl)piperidin-1-yl)thiazole-4-carbonyl)-O-(tert-butyldimethylsilyl)-L-seryl)-L-serinate as a colorless oil (0.309 g, 61%). ¹H NMR (400 MHz, CDCl₃): δ 7.99 (d, J=7.1 Hz, 1H), 7.43 (d, J=7.8 Hz, 1H), 7.39 (d, J=3.0 Hz, 1H), 6.52 (s, 1H), 4.88 (td, J=9.4, 7.7, 5.5 Hz, 2H), 4.59 (td, J=7.2, 3.6 Hz, 1H), 4.53-4.44 (m, 1H), 4.31 (dd, J=11.4, 3.7 Hz, 1H), 4.23-4.16 (m, 1H), 4.03 (dd, J=17.2, 13.6 Hz, 2H), 3.79-3.69 (m, 1H), 3.76 (s, 3H), 3.42-3.34 (m, 2H), 3.30 (q, J=5.7, 5.0 Hz, 2H), 3.04 (td, J=12.4, 3.0 Hz, 2H), 2.31 (tt, J=11.4, 3.6 Hz, 1H), 2.02 (s, 3H), 2.00-1.90 (m, 2H), 1.88-1.74 (m, 2H), 1.45 (s, 9H), 0.92 (s, 9H), 0.14 (s, 3H), 0.13 (s, 3H).

### Methyl 2-(2-(2-(4-((2-((tert-butoxycarbonyl)amino)ethyl)carbamoyl)piperidin-1-yl)thiazole-4-carboxamido)acrylamido)acrylate (15)

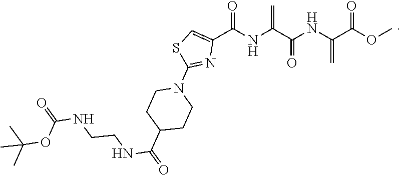 [SMILES: C=C(NC(=O)c1csc(N2CCC(C(=O)NCCNC(=O)OC(C)(C)C)CC2)n1)C(=O)NC(=C)C(=O)OC]

Methyl O-acetyl-N—(N-(2-(4-((2-((tert-butoxycarbonyl)amino)ethyl)carbamoyl)piperidin-1-yl)thiazole-4-carbonyl)-O-(tert-butyldimethylsilyl)-L-seryl)-L-serinate (0.309 g, 0.416 mmole) was dissolved in THE (1 mL). Tetrabutylammonium fluoride (1M solution in THF, 0.950 mL, 0.950 mmole) was added and the solution was stirred at RT for 3 hours. Water (25 mL) was added and the mixture was extracted with ethyl acetate (3×10 mL). The combined organics were dried over magnesium sulfate, filtered, and concentrated. The residue was dissolved in DCM (2 mL) and cooled to 0° C. Triethylamine (0.088 mL, 0.628 mmole) was added followed by methanesulfonyl chloride (0.050 mL, 0.646 mmole). The mixture was stirred at 0° for 2 hours and water (25 mL) was added. The mixture was extracted with DCM (3×10 mL). The combined organics were dried over magnesium sulfate, filtered, and concentrated. The residue was dissolved in THE (2 mL) and cooled to 0° C. 1,8-Diazabicyclo[5.4.0]undec-7-ene (0.094 mL, 0.629 mmole) was added and the solution was stirred at 0° for 2 hours. Water (25 mL) was added and the mixture was extracted with ethyl acetate (3×10 mL). The combined organics were dried over magnesium sulfate, filtered, and concentrated. The crude residue was purified by silica gel chromatography to provide methyl 2-(2-(2-(4-((2-((tert-butoxycarbonyl)amino)ethyl)carbamoyl)piperidin-1-yl)thiazole-4-carboxamido)acrylamido)acrylate as a white solid (0.046 g, 20%). ¹H NMR (400 MHz, CDCl₃): δ 9.72 (s, 1H), 8.51 (s, 1H), 7.42 (s, 1H), 6.72 (d, J=2.1 Hz, 1H), 6.66 (s, 1H), 6.49 (s, 1H), 6.00 (d, J=1.3 Hz, 1H), 5.43 (t, J=1.9 Hz, 1H), 4.88 (s, 1H), 4.06 (dt, J=13.0, 3.7 Hz, 2H), 3.89 (s, 3H), 3.43-3.34 (m, 2H), 3.32 (d, J=7.1 Hz, 2H), 3.08 (ddd, J=13.0, 11.8, 3.1 Hz, 2H), 2.32 (tt, J=11.4, 3.7 Hz, 1H), 1.99 (d, J=3.5 Hz, 2H), 1.90-1.76 (m, 2H), 1.44 (s, 9H).

### Compound 16: Methyl 2-(2-(2-(4-((6-((tert-butoxycarbonyl)amino)hexanamido)methyl)phenyl)thiazole-4-carboxamido)acrylamido)acrylate

### Tert-butyl (4-cyanobenzyl)carbamate (16.1)

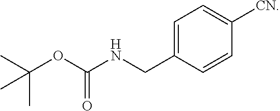 [SMILES: CC(C)(C)OC(=O)NCc1ccc(C#N)cc1]

4-(Aminomethyl)benzonitrile hydrochloride (2.010 g, 11.9 mmole) was suspended in DCM (24 mL). Di-tert-butyl dicarbonate (2.866 g, 13.1 mmole) was added followed by N,N-diisopropylethylamine (4.20 mL, 24.1 mmole) and the mixture was stirred at RT for 3 hours. Water (75 mL) was added and the two layers were separated. The aqueous layer was extracted with DCM (2×15 mL). The combined organics were dried over sodium sulfate, filtered, and concentrated to provide tert-butyl (4-cyanobenzyl)carbamate as a white solid (3.311 g). ¹H NMR (400 MHz, CDCl₃): δ 7.68-7.56 (m, 2H), 7.44-7.34 (m, 2H), 4.97 (s, 1H), 4.37 (d, J=6.2 Hz, 2H), 1.46 (s, 9H)

### Tert-butyl (4-carbamothioylbenzyl)carbamate (16.2)

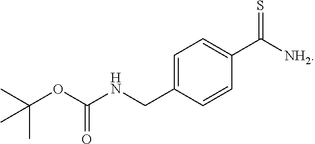 [SMILES: CC(C)(C)OC(=O)NCc1ccc(C(N)=S)cc1]

Tert-butyl (4-cyanobenzyl)carbamate (3.311 g, 14.3 mmole) was dissolved in pyridine (14 mL). Triethylamine (2.20 mL, 15.7 mmole) was added followed by ammonium sulfide (40% aqueous solution, 3.00 mL, 17.6 mmole). The solution was heated to 50° C. for 4 hours and was cooled to RT and concentrated. The residue was dissolved in ethyl acetate (50 mL) and washed sequentially with 1N aq. HCl (3×25 mL) and sat. aq. sodium chloride (1×25 mL). The organics were dried over magnesium sulfate, filtered, and concentrated to provide tert-butyl (4-carbamothioylbenzyl)carbamate as a yellow solid (3.186 g, 100% from 4-(aminomethyl)benzonitrile hydrochloride). ¹H NMR (400 MHz, DMSO-d₆): δ 9.80 (s, 1H), 9.44 (s, 1H), 7.84 (d, J=8.2 Hz, 2H), 7.44 (t, J=6.2 Hz, 1H), 7.25 (d, J=8.2 Hz, 2H), 4.15 (d, J=6.2 Hz, 2H), 1.39 (s, 9H).

### Ethyl 2-(4-(aminomethyl)phenyl)thiazole-4-carboxylate hydrobromide (16.3)

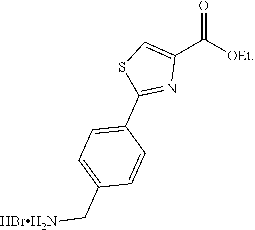

Tert-butyl (4-carbamothioylbenzyl)carbamate (2.623 g, 9.85 mmole) was dissolved in ethanol (20 mL). Ethyl bromopyruvate (1.50 mL, 12.0 mmole) was added and the mixture was heated to 80° C. for 3 hours. The mixture was cooled to RT and the precipitated solid was collected by filtration, washed with a small amount of ethanol, and allowed to air dry to provide ethyl 2-(4-(aminomethyl)phenyl)thiazole-4-carboxylate hydrobromide as a white solid (2.408 g, 71%). ¹H NMR (400 MHz, DMSO-d₆): δ 8.62 (s, 1H), 8.42-8.20 (m, 3H), 8.10-8.01 (m, 2H), 7.69-7.60 (m, 2H), 4.35 (q, J=7.1 Hz, 2H), 4.20-4.09 (m, 2H), 1.34 (t, J=7.1 Hz, 3H).

### Ethyl 2-(4-((6-((tert-butoxycarbonyl)amino)hexanamido)methyl)phenyl)thiazole-4-carboxylate (16.4)

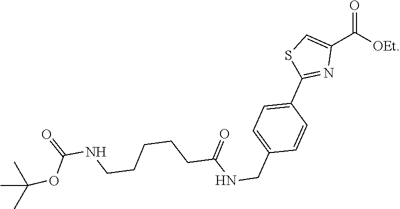 [SMILES: CCOC(=O)c1csc(-c2ccc(CNC(=O)CCCCCNC(=O)OC(C)(C)C)cc2)n1]

6-((Tert-butoxycarbonyl)amino)hexanoic acid (1.795 g, 7.76 mmole) was dissolved in DCM (14 mL). N,N′-Carbonyldiimidazole (1.254 g, 7.73 mmole) was added portionwsie (gas evolution was observed) and the solution was stirred for 60 minutes. Ethyl 2-(4-(aminomethyl)phenyl)thiazole-4-carboxylate hydrobromide (2.408 g, 7.02 mmole) and N,N-diisopropylethylamine (3.70 mL, 21.2 mmole) were added and the solution was stirred at RT for 20 hours. Water (50 mL) was added and the two layers were separated. The aqueous layer was extracted with DCM (2×20 mL). The combined organics were dried over magnesium sulfate, filtered, and concentrated. The crude residue was purified by silica gel chromatography (50-100% ethyl acetate/hexane gradient) to provide ethyl 2-(4-((6-((tert-butoxycarbonyl)amino)hexanamido)methyl)phenyl)thiazole-4-carboxylate as a white solid (3.060 g, 92%). ¹H NMR (400 MHz, CDCl₃): δ 8.15 (s, 1H), 8.01-7.92 (m, 2H), 7.40-7.31 (m, 2H), 5.97 (s, 1H), 4.56 (s, 1H), 4.48 (d, J=5.9 Hz, 2H), 4.45 (q, J=7.1 Hz, 2H), 3.10 (q, J=6.7 Hz, 2H), 2.25 (t, J=7.5 Hz, 2H), 1.76-1.64 (m, 2H), 1.50 (ddd, J=14.3, 7.4, 3.9 Hz, 2H), 1.45-1.40 (m, 12H), 1.40-1.31 (m, 2H).

### 2-(4-((6-((Tert-butoxycarbonyl)amino)hexanamido)methyl)phenyl)thiazole-4-carboxylic acid (16.5)

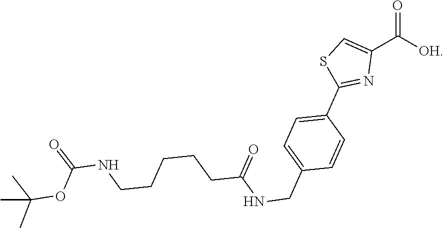 [SMILES: CC(C)(C)OC(=O)NCCCCCC(=O)NCc1ccc(-c2nc(C(=O)O)cs2)cc1]

Using the procedure described in Example 1.3, ethyl 2-(4-((6-((tert-butoxycarbonyl)amino)hexanamido)methyl)phenyl)thiazole-4-carboxylate (3.060 g, 6.43 mmole) was converted to 2-(4-((6-((tert-butoxycarbonyl)amino)hexanamido)methyl)phenyl)thiazole-4-carboxylic acid as a white solid (2.756 g, 96%). ¹H NMR (400 MHz, DMSO-d₆): δ 8.48 (s, 1H), 8.39 (t, J=6.0 Hz, 1H), 7.94 (d, J=8.3 Hz, 2H), 7.43-7.33 (m, 2H), 6.76 (t, J=5.8 Hz, 1H), 4.32 (d, J=5.9 Hz, 2H), 2.90 (q, J =6.6 Hz, 2H), 2.15 (t, J=7.4 Hz, 2H), 1.53 (p, J=7.5 Hz, 2H), 1.37 (s, 11H), 1.24 (td, J=8.4, 4.3 Hz, 2H).

### Methyl N-(2-(4-((6-((tert-butoxycarbonyl)amino)hexanamido)methyl)phenyl)thiazole-4-carbonyl)-O-(tert-butyldimethylsilyl)-L-serinate (16.6)

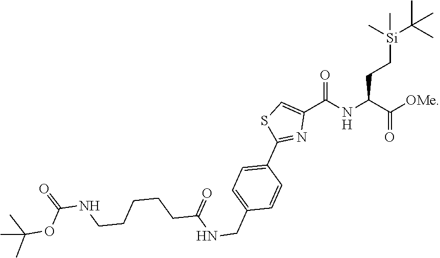 [SMILES: COC(=O)[C@H](CC[Si](C)(C)C(C)(C)C)NC(=O)c1csc(-c2ccc(CNC(=O)CCCCCNC(=O)OC(C)(C)C)cc2)n1]

Using the procedure described in Example 12.3, 2-(4-((6-((tert-butoxycarbonyl)amino)hexanamido)methyl)phenyl)thiazole-4-carboxylic acid (2.756 g, 6.16 mmole) was converted to methyl N-(2-(4-((6-((tert-butoxycarbonyl)amino)hexanamido)methyl)phenyl)thiazole-4-carbonyl)-O-(tert-butyldimethylsilyl)-L-serinate as a colorless oil (3.291 g, 77%). ¹H NMR (400 MHz, CDCl₃): δ 8.21 (d, J=8.7 Hz, 1H), 8.07 (s, 1H), 7.96-7.87 (m, 2H), 7.40-7.31 (m, 2H), 6.01 (s, 1H), 4.90-4.80 (m, 1H), 4.57 (s, 1H), 4.49 (d, J=5.8 Hz, 2H), 4.21 (dd, J=10.0, 2.7 Hz, 1H), 3.95 (dd, J=10.1, 3.4 Hz, 1H), 3.79 (s, 3H), 3.11 (q, J=6.7 Hz, 2H), 2.26 (t, J=7.5 Hz, 2H), 1.78-1.66 (m, 2H), 1.56-1.47 (m, 2H), 1.43 (s, 9H), 1.41-1.33 (m, 2H), 0.92 (s, 9H), 0.08 (s, 3H), 0.06 (s, 3H).

### N-(2-(4-((6-((tert-butoxycarbonyl)amino)hexanamido)methyl)phenyl)thiazole-4-carbonyl)-O-(tert-butyldimethylsilyl)-L-serine (16.7)

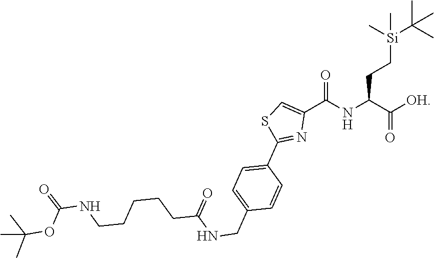 [SMILES: CC(C)(C)OC(=O)NCCCCCC(=O)NCc1ccc(-c2nc(C(=O)N[C@@H](CC[Si](C)(C)C(C)(C)C)C(=O)O)cs2)cc1]

Using the procedure described in Example 4.3, methyl N-(2-(4-((6-((tert-butoxycarbonyl)amino)hexanamido)methyl)phenyl)thiazole-4-carbonyl)-O-(tert-butyldimethylsilyl)-L-serinate (3.291 g, 4.96 mmole) was converted to N-(2-(4-((6-((tert-butoxycarbonyl)amino)hexanamido)methyl)phenyl)thiazole-4-carbonyl)-O-(tert-butyldimethylsilyl)-L-serine as a white solid (3.097 g, 96%). ¹H NMR (400 MHz, DMSO-d₆): δ 8.42-8.36 (m, 1H), 8.37 (s, 1H), 8.18 (d, J=8.6 Hz, 1H), 8.00-7.90 (m, 2H), 7.42-7.35 (m, 2H), 6.77 (d, J=6.1 Hz, 1H), 4.59 (dt, J=8.6, 3.5 Hz, 1H), 4.33 (d, J=5.9 Hz, 2H), 4.12 (dd, J=10.3, 3.4 Hz, 1H), 3.96 (dd, J=10.4, 3.7 Hz, 1H), 2.89 (q, J=6.6 Hz, 2H), 2.15 (t, J=7.4 Hz, 2H), 1.53 (p, J=7.5 Hz, 2H), 1.37 (s, 11H), 1.31-1.20 (m, 2H), 0.88 (s, 9H), 0.06 (s, 3H), 0.04 (s, 3H).

### Methyl N-(2-(4-((6-((tert-butoxycarbonyl)amino)hexanamido)methyl)phenyl)thiazole-4-carbonyl)-O-(tert-butyldimethylsilyl)-L-seryl-L-serinate (16.8)

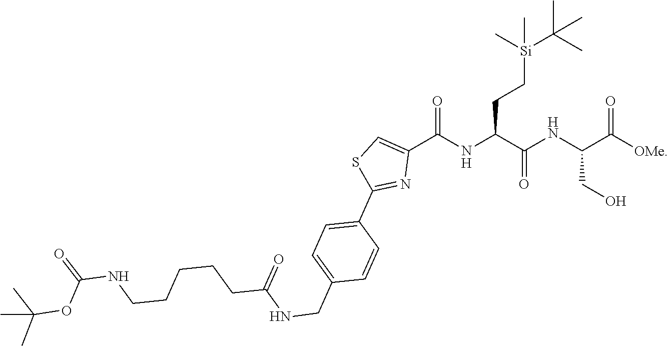 [SMILES: COC(=O)[C@H](CO)NC(=O)[C@H](CC[Si](C)(C)C(C)(C)C)NC(=O)c1csc(-c2ccc(CNC(=O)CCCCCNC(=O)OC(C)(C)C)cc2)n1]

N-(2-(4-((6-((Tert-butoxycarbonyl)amino)hexanamido)methyl)phenyl)thiazole-4-carbonyl)-O-(tert-butyldimethylsilyl)-L-serine (3.097 g, 4.77 mmole) was dissolved in DCM (9 mL). L-Serine methyl ester hydrochloride (0.898 g, 5.77 mmole) was added followed by N,N-diisopropylethylamine (1.70 mL, 9.76 mmole), HOBt·H₂O (0.881 g, 5.75 mmole), and EDC·HCl (1.101 g, 5.74 mmole). The mixture was stirred at RT for 18 hours. Water (50 mL) was added and the two layers were separated. The aqueous layer was extracted with DCM (2×15 mL). The combined organics were dried over magnesium sulfate, filtered, and concentrated. The crude residue was purified by silica gel chromatography (80-100% ethyl acetate/hexane gradient) to provide methyl N-(2-(4-((6-((tert-butoxycarbonyl)amino)hexanamido)methyl)phenyl)thiazole-4-carbonyl)-O-(tert-butyldimethylsilyl)-L-seryl-L-serinate as a white solid (2.354 g, 66%). ¹H NMR (400 MHz, CDCl₃): δ 8.24 (d, J=7.1 Hz, 1H), 8.06 (s, 1H), 7.89-7.84 (m, 2H), 7.52 (d, J=7.5 Hz, 1H), 7.35-7.29 (m, 2H), 6.15-6.02 (m, 1H), 4.74-4.63 (m, 2H), 4.61 (d, J=13.7 Hz, 1H), 4.48 (d, J=6.1 Hz, 2H), 4.19 (dd, J=9.8, 4.1 Hz, 1H), 4.05-3.95 (m, 2H), 3.88-3.82 (m, 1H), 3.78 (d, J=9.3 Hz, 4H), 3.11 (q, J=6.7 Hz, 3H), 2.26 (t, J=7.6 Hz, 2H), 1.75-1.66 (m, 3H), 1.55-1.46 (m, 2H), 1.43 (s, 10H), 1.41-1.32 (m, 2H), 0.94 (d, J=1.1 Hz, 9H), 0.14 (s, 3H), 0.12 (s, 3H).

### Methyl (2-(4-((6-((tert-butoxycarbonyl)amino)hexanamido)methyl)phenyl)thiazole-4-carbonyl)-L-seryl-L-serinate (16.9)

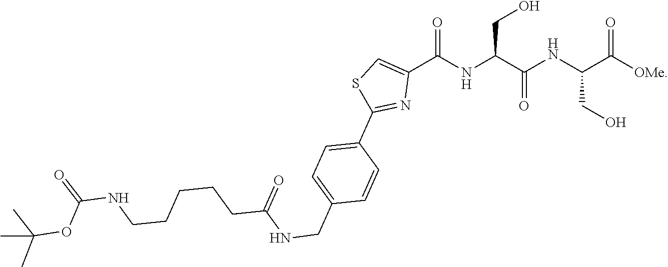 [SMILES: COC(=O)[C@H](CO)NC(=O)[C@H](CO)NC(=O)c1csc(-c2ccc(CNC(=O)CCCCCNC(=O)OC(C)(C)C)cc2)n1]

N-(2-(4-((6-((Tert-butoxycarbonyl)amino)hexanamido)methyl)phenyl)thiazole-4-carbonyl)-O-(tert-butyldimethylsilyl)-L-seryl-L-serinate (2.354 g, 3.14 mmole) was dissolved in THE (6 mL). Tetrabutylammonium fluoride (1M solution in THF, 3.50 mL, 3.50 mmole) was added and the solution was stirred at RT for 2 hours. Water (50 mL) was added and the precipitated solid was collected by filtration, washed with water and allowed to air dry to provide methyl (2-(4-((6-((tert-butoxycarbonyl)amino)hexanamido)methyl)phenyl)thiazole-4-carbonyl)-L-seryl-L-serinate as a white solid (1.790 g, 90%). ¹H NMR (400 MHz, DMSO-d₆): δ 8.49-8.41 (m, 2H), 8.38 (d, J=2.7 Hz, 1H), 8.31 (dd, J=8.1, 2.6 Hz, 1H), 8.08-7.99 (m, 2H), 7.49-7.40 (m, 2H), 6.80 (d, J=6.0 Hz, 1H), 4.68 (dt, J=8.2, 5.2 Hz, 1H), 4.46 (dt, J=7.8, 4.6 Hz, 1H), 4.37 (d, J=6.0 Hz, 2H), 3.86-3.73 (m, 3H), 3.68 (d, J=4.2 Hz, 4H), 2.93 (q, J=6.6 Hz, 2H), 2.19 (t, J=7.4 Hz, 2H), 1.57 (p, J=7.5 Hz, 2H), 1.41 (s, 11H), 1.33-1.22 (m, 2H).

### Methyl 2-(2-(2-(4-((6-((tert-butoxycarbonyl)amino)hexanamido)methyl)phenyl)thiazole-4-carboxamido)acrylamido)acrylate (16)

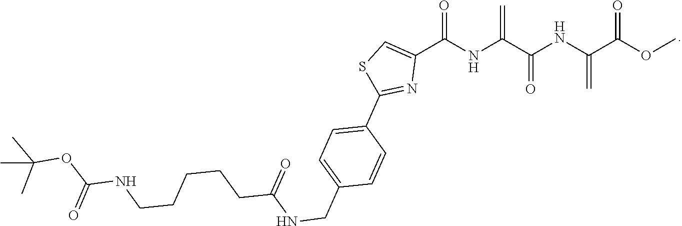 [SMILES: C=C(NC(=O)c1csc(-c2ccc(CNC(=O)CCCCCNC(=O)OC(C)(C)C)cc2)n1)C(=O)NC(=C)C(=O)OC]

Methyl (2-(4-((6-((tert-butoxycarbonyl)amino)hexanamido)methyl)phenyl)thiazole-4-carbonyl)-L-seryl-L-serinate (0.306 g, 0.481 mmole) was dissolved in DMF (2 mL) and cooled to 0° C. Triethylamine (0.200 mL, 1.43 mmole) was added followed by methanesulfonyl chloride (0.110 mL, 1.42 mmole). The mixture was stirred at 0° for 60 minutes and water (25 mL) was added. The mixture was extracted with ethyl acetate (3×10 mL). The combined organics were dried over magnesium sulfate, filtered, and concentrated. The residue was suspended in THE and cooled to 0° C. 1,8-Diazabicyclo[5.4.0]undec-7-ene (0.210 mL, 1.40 mmole) was added and the mixture was stirred at 0° for 60 minutes. Water (25 mL) was added and the mixture was extracted with ethyl acetate (3×10 mL). The combined organics were dried over magnesium sulfate, filtered, and concentrated. The crude residue was purified by silica gel chromatography (80-100% ethyl acetate/hexane gradient) to provide a white solid. The solid was triturated with ethyl acetate and collected by filtration to provide methyl 2-(2-(2-(4-((6-((tert-butoxycarbonyl)amino)hexanamido)methyl)phenyl)thiazole-4-carboxamido)acrylamido)acrylate as a white solid (0.014 g, 4.9%). ¹H NMR (400 MHz, DMSO-d₆): δ 9.97 (s, 1H), 9.95 (s, 1H), 8.48 (s, 1H), 8.38 (m, 1H), 8.02-7.91 (m, 2H), 7.46-7.36 (m, 2H), 6.76 (s, 1H), 6.51 (d, J=1.3 Hz, 1H), 5.87 (d, J=1.4 Hz, 1H), 5.87 (s, 1H), 5.84 (s, 1H), 4.32 (d, J=5.9 Hz, 2H), 3.75 (s, 3H), 2.89 (q, J=6.6 Hz, 2H), 2.15 (t, J=7.5 Hz, 2H), 1.53 (p, J=7.5 Hz, 2H), 1.36 (s, 11H), 1.24 (q, J=8.4, 7.6 Hz, 2H).

### Compound 17: Methyl 2-(2-(2-morpholinothiazole-4-carboxamido)acrylamido)acrylate

### Ethyl 2-morpholinothiazole-4-carboxylate (17.1)

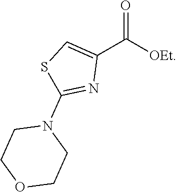 [SMILES: CCOC(=O)c1csc(N2CCOCC2)n1]

Ethyl 2-bromothiazole-4-carboxylate (1.009 g, 4.27 mmole) was dissolved in N,N-dimethylacetamide (8 mL). Morpholine (0.410 mL, 4.69 mmole) and triethylamine (0.710 mL, 5.07 mmole) were added and the mixture was heated to 80° C. for 24 hours then was cooled to RT. Water (50 mL) was added and the mixture was extracted with ethyl acetate (3×15 mL). The combined organics were dried over magnesium sulfate, filtered, and concentrated. The crude residue was purified by silica gel chromatography (20-40% ethyl acetate/hexane gradient) to provide ethyl 2-morpholinothiazole-4-carboxylate as a pale yellow solid (0.721 g, 70%). ¹H NMR (400 MHz, CDCl₃): δ 7.48 (s, 1H), 4.36 (q, J=7.1 Hz, 2H), 3.86-3.77 (m, 4H), 3.57-3.47 (m, 4H), 1.37 (t, J=7.2 Hz, 3H).

### 2-Morpholinothiazole-4-carboxylic acid (17.2)

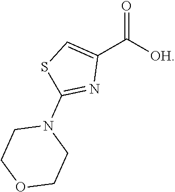 [SMILES: O=C(O)c1csc(N2CCOCC2)n1]

Using the procedure described in Example 1.3, ethyl 2-morpholinothiazole-4-carboxylate (0.721 g, 2.96 mmole) was converted to 2-morpholinothiazole-4-carboxylic acid as a white solid (0.341 g, 54%). ¹H NMR (400 MHz, DMSO-d₆): δ 12.62 (s, 1H), 7.67 (s, 1H), 3.74-3.64 (m, 4H), 3.39 (ddd, J=6.3, 4.9, 3.5 Hz, 4H).

### Methyl O-(tert-butyldimethylsilyl)-N-(2-morpholinothiazole-4-carbonyl)-L-serinate (17.3)

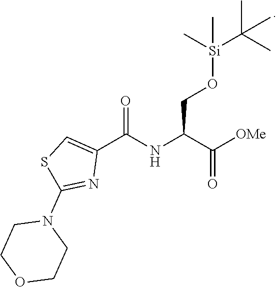 [SMILES: COC(=O)[C@H](CO[Si](C)(C)C(C)(C)C)NC(=O)c1csc(N2CCOCC2)n1]

Using the procedure described in Example 12.3, 2-morpholinothiazole-4-carboxylic acid (0.341 g, 1.59 mmole) was converted to methyl O-(tert-butyldimethylsilyl)-N-(2-morpholinothiazole-4-carbonyl)-L-serinate as a colorless oil (0.494 g, 72%). ¹H NMR (400 MHz, CDCl₃): δ 7.95 (d, J=8.8 Hz, 1H), 7.43 (s, 1H), 4.79 (ddd, J=8.8, 3.4, 2.6 Hz, 1H), 4.20-4.13 (m, 1H), 3.85-3.79 (m, 6H), 3.76 (s, 3H), 3.56-3.50 (m, 2H), 3.48 (q, J=4.9 Hz, 4H), 0.88 (s, 9H), 0.04 (s, 3H), 0.03 (s, 3H).

O-(tert-butyldimethylsilyl)-N-(2-morpholinothiazole-4-carbonyl)-L-serine (17.4):

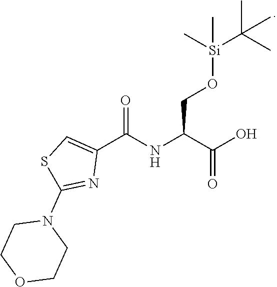 [SMILES: CC(C)(C)[Si](C)(C)OC[C@H](NC(=O)c1csc(N2CCOCC2)n1)C(=O)O]

Using the procedure described in Example 4.3, methyl O-(tert-butyldimethylsilyl)-N-(2-morpholinothiazole-4-carbonyl)-L-serinate (0.494 g, 1.15 mmole) was converted to O-(tert-butyldimethylsilyl)-N-(2-morpholinothiazole-4-carbonyl)-L-serine as a colorless gel (0.396 g, 83%). ¹H NMR (400 MHz, DMSO-d₆): δ 7.87 (d, J=8.6 Hz, 1H), 7.56 (d, J=6.1 Hz, 1H), 4.53 (dt, J=8.7, 3.3 Hz, 1H), 4.13-4.08 (m, 1H), 3.91 (dd, J=10.3, 3.6 Hz, 1H), 3.81-3.71 (m, 4H), 3.51-3.41 (m, 4H), 0.89 (d, J=1.9 Hz, 9H), 0.07 (s, 3H), 0.06 (s, 3H).

### Methyl O-acetyl-N—(O-(tert-butyldimethylsilyl)-N-(2-morpholinothiazole-4-carbonyl)-L-seryl)-L-serinate (17.5)

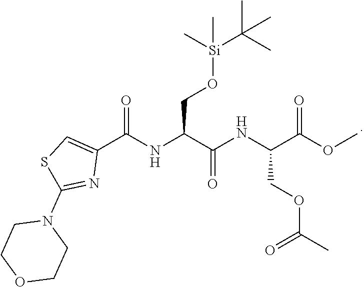 [SMILES: COC(=O)[C@H](COC(C)=O)NC(=O)[C@H](CO[Si](C)(C)C(C)(C)C)NC(=O)c1csc(N2CCOCC2)n1]

Using the procedure described in Example 15.6, O-(tert-butyldimethylsilyl)-N-(2-morpholinothiazole-4-carbonyl)-L-serine (0.396 g, 0.953 mmole) was converted to methyl O-acetyl-N—(O-(tert-butyldimethylsilyl)-N-(2-morpholinothiazole-4-carbonyl)-L-seryl)-L-serinate as a colorless oil (0.339 g, 64%). ¹H NMR (400 MHz, CDCl₃): δ 7.99 (d, J=7.1 Hz, 1H), 7.43 (d, J=2.2 Hz, 2H), 4.91-4.82 (m, 1H), 4.63-4.54 (m, 1H), 4.53-4.41 (m, 1H), 4.35 (ddd, J=26.4, 11.4, 3.7 Hz, 1H), 4.18 (ddd, J=9.8, 3.7, 0.9 Hz, 1H), 3.87-3.79 (m, 5H), 3.76 (s, 3H), 3.53-3.44 (m, 4H), 2.03 (s, 3H), 0.92 (d, J=1.0 Hz, 9H), 0.15 (s, 3H), 0.12 (s, 3H).

### Methyl 2-(2-(2-morpholinothiazole-4-carboxamido)acrylamido)acrylate (17)

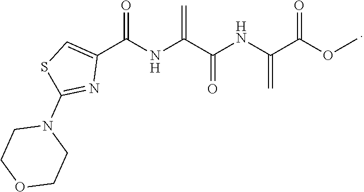 [SMILES: C=C(NC(=O)c1csc(N2CCOCC2)n1)C(=O)NC(=C)C(=O)OC]

Using the procedure described in Compound 15, methyl O-acetyl-N—(O-(tert-butyldimethylsilyl)-N-(2-morpholinothiazole-4-carbonyl)-L-seryl)-L-serinate (0.339 g, 0.607 mmole) was converted to methyl 2-(2-(2-morpholinothiazole-4-carboxamido)acrylamido)acrylate as a white solid (0.093 g, 42%). ¹H NMR (400 MHz, CDCl₃): δ 9.74 (s, 1H), 8.51 (s, 1H), 7.47 (s, 1H), 6.73 (d, J=2.2 Hz, 1H), 6.65 (s, 1H), 6.00 (d, J=1.3 Hz, 1H), 5.44 (t, J=1.9 Hz, 1H), 3.89 (s, 3H), 3.86-3.77 (m, 4H), 3.57-3.46 (m, 4H).

### Compound 18: Tert-butyl 4-(4-((3-((3-methoxy-3-oxoprop-1-en-2-yl)amino)-3-oxoprop-1-en-2-yl)carbamoyl)thiazol-2-yl)piperazine-1-carboxylate

### Ethyl 2-(4-(tert-butoxycarbonyl)piperazin-1-yl)thiazole-4-carboxylate (18.1)

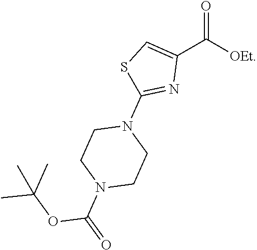 [SMILES: CCOC(=O)c1csc(N2CCN(C(=O)OC(C)(C)C)CC2)n1]

Ethyl 2-bromothiazole-4-carboxylate (1.001 g, 4.24 mmole) was dissolved in N,N-dimethylacetamide (8 mL). Tert-butyl piperazine-1-carboxylate (1.182 g, 6.35 mmole) was added followed by trimethylamine (0.720 mL, 5.14 mmole), and the mixture was heated to 80° C. for 3 days, then was cooled to RT. Water (50 mL) was added and the mixture was extracted with ethyl acetate (3×15 mL). The combined organics were dried over magnesium sulfate, filtered, and concentrated. The crude residue was purified by silica gel chromatography (10-40% ethyl acetate/hexane gradient) to provide ethyl 2-(4-(tert-butoxycarbonyl)piperazin-1-yl)thiazole-4-carboxylate as a pale yellow solid (1.325 g, 92%). ¹H NMR (400 MHz, CDCl₃): δ 7.47 (s, 1H), 4.36 (q, J=7.1 Hz, 2H), 3.59-3.47 (m, 8H), 1.48 (s, 9H), 1.37 (t, J=7.1 Hz, 3H).

### 2-(4-(Tert-butoxycarbonyl)piperazin-1-yl)thiazole-4-carboxylic acid (18.2)

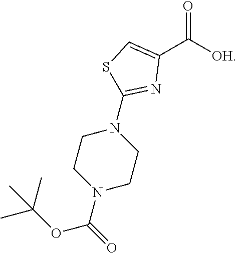 [SMILES: CC(C)(C)OC(=O)N1CCN(c2nc(C(=O)O)cs2)CC1]

Using the procedure described in Example 1.3, ethyl 2-(4-(tert-butoxycarbonyl)piperazin-1-yl)thiazole-4-carboxylate (1.325 g, 3.88 mmole) was converted to 2-(4-(tert-butoxycarbonyl)piperazin-1-yl)thiazole-4-carboxylic acid as a white solid (1.187 g, 98%). ¹H NMR (400 MHz, DMSO-d₆): δ 12.63 (s, 1H), 7.66 (s, 1H), 3.52-3.36 (m, 8H), 1.42 (s, 9H)

### Tert-butyl (S)-4-(4-((3-((tert-butyldimethylsilyl)oxy)-1-methoxy-1-oxopropan-2-yl)carbamoyl)thiazol-2-yl)piperazine-1-carboxylate (18.3)

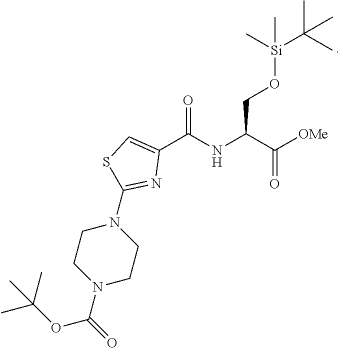 [SMILES: COC(=O)[C@H](CO[Si](C)(C)C(C)(C)C)NC(=O)c1csc(N2CCN(C(=O)OC(C)(C)C)CC2)n1]

Using the procedure described in Example 15.3, 2-(4-(tert-butoxycarbonyl)piperazin-1-yl)thiazole-4-carboxylic acid (1.187 g, 3.79 mmole) was converted to tert-butyl (S)-4-(4-((3-((tert-butyldimethylsilyl)oxy)-1-methoxy-1-oxopropan-2-yl)carbamoyl)thiazol-2-yl)piperazine-1-carboxylate as a white solid (1.920 g, 96%). ¹H NMR (400 MHz, CDCl₃): δ 7.94 (d, J=8.7 Hz, 1H), 7.42 (s, 1H), 4.78 (ddd, J=8.8, 3.4, 2.6 Hz, 1H), 4.19-4.14 (m, 1H), 3.89 (dd, J=10.0, 3.4 Hz, 1H), 3.76 (s, 3H), 3.56 (t, J=5.1 Hz, 4H), 3.48 (q, J=5.3, 4.5 Hz, 4H), 1.49 (s, 9H), 0.88 (s, 9H), 0.05 (s, 3H), 0.03 (s, 3H).

### N-(2-(4-(Tert-butoxycarbonyl)piperazin-1-yl)thiazole-4-carbonyl)-O-(tert-butyldimethylsilyl)-L-serine (18.4)

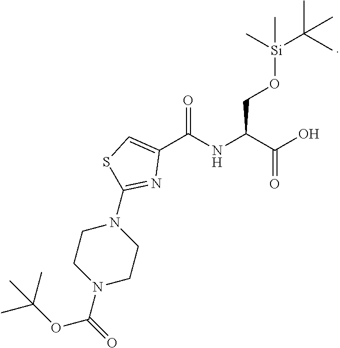 [SMILES: CC(C)(C)OC(=O)N1CCN(c2nc(C(=O)N[C@@H](CO[Si](C)(C)C(C)(C)C)C(=O)O)cs2)CC1]

Using the procedure described in Example 4.3, tert-butyl (S)-4-(4-((3-((tert-butyldimethylsilyl)oxy)-1-methoxy-1-oxopropan-2-yl)carbamoyl)thiazol-2-yl)piperazine-1-carboxylate (1.920 g, 3.63 mmole) was converted to N-(2-(4-(tert-butoxycarbonyl)piperazin-1-yl)thiazole-4-carbonyl)-O-(tert-butyldimethylsilyl)-L-serine as a white solid (1.922 g). ¹H NMR (400 MHz, DMSO-d₆): δ 7.87 (d, J=8.6 Hz, 1H), 7.55 (d, J=6.8 Hz, 1H), 4.53 (dt, J=8.7, 3.3 Hz, 1H), 4.15-4.09 (m, 1H), 3.91 (dd, J=10.3, 3.6 Hz, 1H), 3.52 (s, 8H), 1.47 (s, 9H), 0.89 (s, 9H), 0.08 (s, 3H), 0.06 (s, 3H)

### Tert-butyl 4-(4-(((6S,9S)-9-(methoxycarbonyl)-2,2,3,3-tetramethyl-7,12-dioxo-4,11-dioxa-8-aza-3-silatridecan-6-yl)carbamoyl)thiazol-2-yl)piperazine-1-carboxylate (18.5)

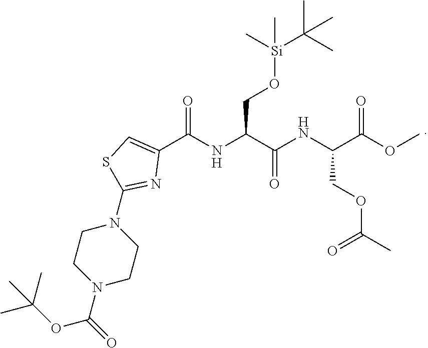 [SMILES: COC(=O)[C@H](COC(C)=O)NC(=O)[C@H](CO[Si](C)(C)C(C)(C)C)NC(=O)c1csc(N2CCN(C(=O)OC(C)(C)C)CC2)n1]

Using the procedure described in Example 15.6, N-(2-(4-(tert-butoxycarbonyl)piperazin-1-yl)thiazole-4-carbonyl)-O-(tert-butyldimethylsilyl)-L-serine (1.922 g, 3.73 mmole) was converted to tert-butyl 4-(4-(((6S,9S)-9-(methoxycarbonyl)-2,2,3,3-tetramethyl-7,12-dioxo-4,11-dioxa-8-aza-3-silatridecan-6-yl)carbamoyl)thiazol-2-yl)piperazine-1-carboxylate as a colorless oil (1.481 g, 60%). ¹H NMR (400 MHz, CDCl₃): δ 7.99 (d, J=7.0 Hz, 1H), 7.48-7.39 (m, 2H), 4.88 (dt, J=7.6, 3.7 Hz, 1H), 4.58 (td, J=7.2, 3.7 Hz, 1H), 4.52-4.45 (m, 1H), 4.32 (dd, J=11.4, 3.6 Hz, 1H), 4.18 (dd, J=9.8, 3.7 Hz, 1H), 3.76 (d, J=2.0 Hz, 3H), 3.73 (d, J=9.7 Hz, 1H), 3.56 (dq, J=5.6, 2.6 Hz, 4H), 3.52-3.43 (m, 4H), 2.05 (s, 3H), 1.49 (s, 9H), 0.93 (s, 9H), 0.14 (s, 3H), 0.13 (s, 3H)

### Tert-butyl 4-(4-((3-((3-methoxy-3-oxoprop-1-en-2-yl)amino)-3-oxoprop-1-en-2-yl)carbamoyl)thiazol-2-yl)piperazine-1-carboxylate (18)

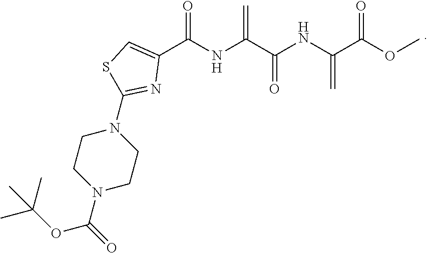 [SMILES: C=C(NC(=O)c1csc(N2CCN(C(=O)OC(C)(C)C)CC2)n1)C(=O)NC(=C)C(=O)OC]

Using the procedure described for Compound 15, tert-butyl 4-(4-(((6S,9S)-9-(methoxycarbonyl)-2,2,3,3-tetramethyl-7,12-dioxo-4,11-dioxa-8-aza-3-silatridecan-6-yl)carbamoyl)thiazol-2-yl)piperazine-1-carboxylate (1.481 g, 2.25 mmole) was converted to tert-butyl 4-(4-((3-((3-methoxy-3-oxoprop-1-en-2-yl)amino)-3-oxoprop-1-en-2-yl)carbamoyl)thiazol-2-yl)piperazine-1-carboxylate as a white solid (0.206 g, 20%). ¹H NMR (400 MHz, CDCl₃): δ 9.73 (s, 1H), 8.52 (s, 1H), 7.46 (s, 1H), 6.72 (d, J=2.1 Hz, 1H), 6.66 (s, 1H), 6.01 (d, J=1.3 Hz, 1H), 5.44 (t, J=1.9 Hz, 1H), 3.89 (s, 3H), 3.58 (dd, J=6.6, 3.5 Hz, 4H), 3.51 (dt, J=7.4, 3.5 Hz, 4H), 1.49 (s, 9H).

### Compound 19: Methyl 2-(2-(2-(4-(6-azidohexanamido)piperidin-1-yl)thiazole-4-carboxamido)acrylamido)acrylate

### Ethyl 2-(4-(6-bromohexanamido)piperidin-1-yl)thiazole-4-carboxylate (19.1)

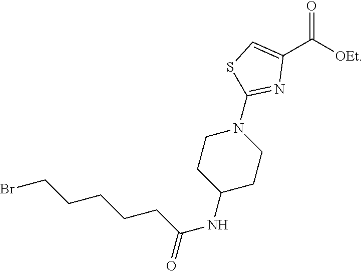 [SMILES: CCOC(=O)c1csc(N2CCC(NC(=O)CCCCCBr)CC2)n1]

6-Bromohexanoic acid (1.212 g, 6.21 mmole) was dissolved in DCM (5.5 mL) and DMF (2 drops) was added. Oxalyl chloride (0.540 mL, 6.19 mmole) was added dropwise (gas evolution was observed) and the solution was stirred at RT for 3 hours, then was concentrated. In a separate flask, ethyl 2-(4-((tert-butoxycarbonyl)amino)piperidin-1-yl)thiazole-4-carboxylate (1.995 g, 5.61 mmole) was dissolved in DCM (11 mL). HCl (4M solution in 1,4-dioxane, 5.60 mL, 22.4 mmole) was added and the mixture was stirred at RT for 3 hours and was concentrated. The residue was dissolved in DCM (11 mL) and cooled to 0° C. N,N-Diisopropylethylamine (2.00 mL, 11.5 mmole) was added followed by slow addition of a solution of the acid chloride generated above in DCM (2 mL). The resulting mixture was stirred at 0° for 15 minutes, then at RT for 18 hours. Water (50 mL) was added and the two layers were separated. The aqueous layer was extracted with DCM (3×15 mL). The combined organics were dried over magnesium sulfate, filtered, and concentrated. The crude residue was purified by silica gel chromatography (10-40% ethyl acetate/hexane gradient) to provide ethyl 2-(4-(6-bromohexanamido)piperidin-1-yl)thiazole-4-carboxylate as a white solid (1.287 g, 53%). ¹H NMR (400 MHz, CDCl₃): δ 7.44 (s, 1H), 5.34 (d, J=7.9 Hz, 1H), 4.35 (q, J=7.1 Hz, 2H), 4.08-3.97 (m, 3H), 3.41 (t, J=6.7 Hz, 2H), 3.17 (ddd, J=13.3, 11.8, 2.9 Hz, 2H), 2.18 (t, J=7.4 Hz, 2H), 2.10-1.99 (m, 2H), 1.88 (dq, J=7.9, 6.8 Hz, 2H), 1.74-1.62 (m, 2H), 1.57-1.42 (m, 4H), 1.37 (t, J=7.1 Hz, 3H).

### Ethyl 2-(4-(6-azidohexanamido)piperidin-1-yl)thiazole-4-carboxylate (19.2)

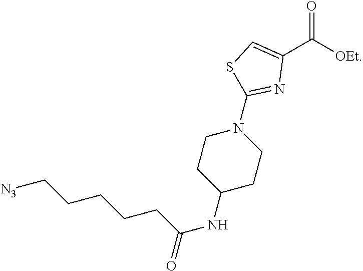 [SMILES: CCOC(=O)c1csc(N2CCC(NC(=O)CCCCCN=[N+]=[N-])CC2)n1]

Ethyl 2-(4-(6-bromohexanamido)piperidin-1-yl)thiazole-4-carboxylate (1.287 g, 2.98 mmole) was dissolved in DMF (6 mL). Sodium azide (0.233 g, 3.58 mmole) was added and the mixture was heated to 80° C. for 18 hours, then was cooled to RT. The mixture was diluted with ethyl acetate (25 mL) and washed with water (3×25 mL). The organics were dried over magnesium sulfate, filtered, and concentrated to provide ethyl 2-(4-(6-azidohexanamido)piperidin-1-yl)thiazole-4-carboxylate as a pale yellow solid (1.127 g, 96%). ¹H NMR (400 MHz, CDCl₃): δ 7.44 (s, 1H), 5.51 (d, J=7.6 Hz, 1H), 4.35 (q, J=7.1 Hz, 2H), 4.01 (dddd, J=11.2, 7.1, 4.4, 2.2 Hz, 3H), 3.28 (t, J=6.8 Hz, 2H), 3.16 (ddd, J=13.4, 11.8, 2.9 Hz, 2H), 2.18 (t, J=7.5 Hz, 2H), 2.04 (d, J=7.3 Hz, 2H), 1.73-1.57 (m, 4H), 1.57-1.46 (m, 2H), 1.46-1.39 (m, 2H), 1.37 (t, J=7.1 Hz, 3H).

### Methyl N-(2-(4-(6-azidohexanamido)piperidin-1-yl)thiazole-4-carbonyl)-O-(tert-butyldimethylsilyl)-L-serinate (19.3)

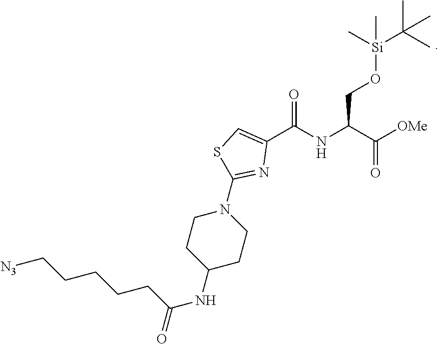 [SMILES: COC(=O)[C@H](CO[Si](C)(C)C(C)(C)C)NC(=O)c1csc(N2CCC(NC(=O)CCCCCN=[N+]=[N-])CC2)n1]

Ethyl 2-(4-(6-azidohexanamido)piperidin-1-yl)thiazole-4-carboxylate (1.127 g, 2.86 mmole) was dissolved in 4/1/1 THF/methanol/water (5.7 mL). Lithium hydroxide monohydrate (0.240 g, 5.72 mmole) was added and the mixture was stirred at RT for 2 hours. Water (40 mL) was added and the solution was treated with 1N aq. HCl to pH=4. The mixture was extracted with ethyl acetate (3×15 mL). The combined organics were dried over magnesium sulfate, filtered, and concentrated. The residue was suspended in DCM (5.7 mL) and L-serine methyl ester hydrochloride (0.535 g, 3.44 mmole) was added. N,N-Diisopropylethylamine (1.00 mL, 5.74 mmole), HOBt·H₂O (0.530 g, 3.46 mmole), and EDC·HCl (0.664 g, 3.46 mmole) were added and the mixture was stirred at RT for 18 hours. Water (25 mL) was added and the mixture was extracted with DCM (3×10 mL). The combined organics were dried over magnesium sulfate, filtered, and concentrated. The residue was dissolved in DCM (5.7 mL). Imidazole (0.215 g, 3.16 mmole) and tert-butyldimethylchlorosilane (0.477 g, 3.16 mmole) were added and the mixture was stirred at RT for 3 hours. Water (25 mL) was added and the mixture was extracted with DCM (3×10 mL). The combined organics were dried over magnesium sulfate, filtered, and concentrated. The crude residue was purified by silica gel chromatography (50-90% ethyl acetate/hexane gradient) to provide methyl N-(2-(4-(6-azidohexanamido)piperidin-1-yl)thiazole-4-carbonyl)-O-(tert-butyldimethylsilyl)-L-serinate as a thick, colorless oil (1.561 g, 94%). ¹H NMR (400 MHz, CDCl₃): δ 7.95 (d, J=8.8 Hz, 1H), 7.38 (s, 1H), 5.44 (d, J=7.9 Hz, 1H), 4.84-4.73 (m, 1H), 4.19-4.14 (m, 1H), 4.07-3.98 (m, 2H), 3.98-3.90 (m, 1H), 3.88 (dd, J=10.0, 3.4 Hz, 1H), 3.76 (s, 3H), 3.28 (t, J=6.8 Hz, 2H), 3.21-3.08 (m, 2H), 2.19 (t, J=7.5 Hz, 2H), 2.03-1.96 (m, 2H), 1.74-1.57 (m, 4H), 1.57-1.47 (m, 2H), 1.47-1.37 (m, 2H), 0.88 (s, 9H), 0.05 (s, 3H), 0.03 (s, 3H).

### Methyl O-acetyl-N—(N-(2-(4-(6-azidohexanamido)piperidin-1-yl)thiazole-4-carbonyl)-O-(tert-butyldimethylsilyl)-L-seryl)-L-serinate (19.4)

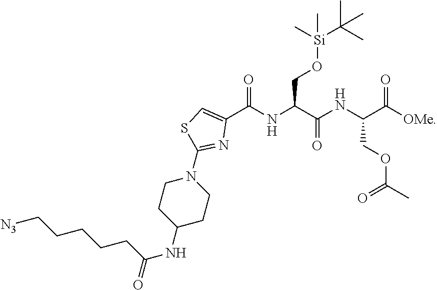 [SMILES: COC(=O)[C@H](COC(C)=O)NC(=O)[C@H](CO[Si](C)(C)C(C)(C)C)NC(=O)c1csc(N2CCC(NC(=O)CCCCCN=[N+]=[N-])CC2)n1]

Using the procedure described in Example 15.6, methyl N-(2-(4-(6-azidohexanamido)piperidin-1-yl)thiazole-4-carbonyl)-O-(tert-butyldimethylsilyl)-L-serinate (1.561 g, 2.68 mmole) was converted to methyl O-acetyl-N—(N-(2-(4-(6-azidohexanamido)piperidin-1-yl)thiazole-4-carbonyl)-O-(tert-butyldimethylsilyl)-L-seryl)-L-serinate as a thick, pale yellow oil (1.312 g, 69%). ¹H NMR (400 MHz, CDCl₃): δ 7.98 (d, J=7.1 Hz, 1H), 7.43 (d, J=7.8 Hz, 1H), 7.40 (d, J=2.6 Hz, 1H), 5.38 (d, J=7.9 Hz, 1H), 4.87 (dq, J=8.0, 4.0 Hz, 1H), 4.58 (td, J=7.2, 3.6 Hz, 1H), 4.52-4.43 (m, 1H), 4.31 (dd, J=11.4, 3.7 Hz, 1H), 4.18 (ddd, J=9.9, 3.7, 1.4 Hz, 1H), 4.08-3.91 (m, 3H), 3.76 (d, J=2.1 Hz, 3H), 3.75-3.69 (m, 1H), 3.28 (t, J=6.8 Hz, 2H), 3.21-3.07 (m, 2H), 2.19 (t, J=7.5 Hz, 2H), 2.01 (s, 3H), 1.73-1.57 (m, 6H), 1.51 (ddd, J=12.6, 9.1, 5.3 Hz, 2H), 1.46-1.38 (m, 2H), 0.92 (s, 9H), 0.14 (s, 3H), 0.12 (s, 3H).

### Methyl 2-(2-(2-(4-(6-azidohexanamido)piperidin-1-yl)thiazole-4-carboxamido)acrylamido)acrylate (19)

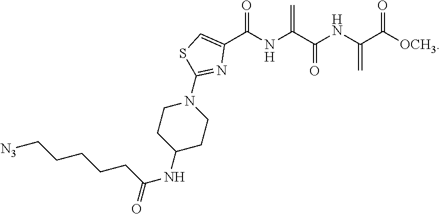 [SMILES: C=C(NC(=O)c1csc(N2CCC(NC(=O)CCCCCN=[N+]=[N-])CC2)n1)C(=O)NC(=C)C(=O)OC]

Using the procedure described for Compound 18, methyl O-acetyl-N—(N-(2-(4-(6-azidohexanamido)piperidin-1-yl)thiazole-4-carbonyl)-O-(tert-butyldimethylsilyl)-L-seryl)-L-serinate (1.312 g, 1.85 mmole) was converted to methyl 2-(2-(2-(4-(6-azidohexanamido)piperidin-1-yl)thiazole-4-carboxamido)acrylamido)acrylate as a white solid (0.481 g, 50%). ¹H NMR (400 MHz, CDCl₃): δ 9.71 (s, 1H), 8.51 (s, 1H), 7.43 (s, 1H), 6.72 (d, J=2.1 Hz, 1H), 6.65 (s, 1H), 6.00 (d, J=1.3 Hz, 1H), 5.44 (t, J=1.9 Hz, 1H), 5.36 (d, J=7.9 Hz, 1H), 4.10-3.96 (m, 3H), 3.89 (s, 3H), 3.28 (t, J=6.8 Hz, 2H), 3.17 (ddd, J=13.3, 11.8, 2.9 Hz, 2H), 2.19 (t, J=7.5 Hz, 2H), 2.11-2.00 (m, 2H), 1.74-1.60 (m, 4H), 1.56-1.46 (m, 2H), 1.46-1.36 (m, 2H).

### Compound 20: Methyl (S)-2-(2-(2-(3-((tert-butoxycarbonyl)amino)piperidin-1-yl)thiazole-4-carboxamido)acrylamido)acrylate

### Ethyl (S)-2-(3-((tert-butoxycarbonyl)amino)piperidin-1-yl)thiazole-4-carboxylate (20.1)

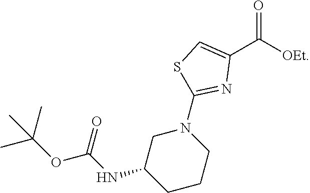 [SMILES: CCOC(=O)c1csc(N2CCC[C@H](NC(=O)OC(C)(C)C)C2)n1]

Ethyl 2-bromothiazole-4-carboxylate (2.003 g, 8.48 mmole) was dissolved in DMA (9 mL). Tert-butyl (S)-piperidin-3-ylcarbamate (1.873 g, 9.35 mmole) was added followed by trimethylamine (1.45 mL, 10.3 mmole), and the mixture was heated to 80° C. for 24 hours, then was cooled to RT and diluted with ethyl acetate (50 mL). The mixture was washed with water (3×25 mL), dried over magnesium sulfate, filtered, and concentrated. The crude residue was purified by silica gel chromatography (30-60% ethyl acetate/hexane gradient) to provide ethyl (S)-2-(3-((tert-butoxycarbonyl)amino)piperidin-1-yl)thiazole-4-carboxylate as a pale yellow solid (2.606 g, 86%). ¹H NMR (400 MHz, CDCl₃): δ 7.44 (s, 1H), 4.71 (s, 1H), 4.35 (q, J=7.1 Hz, 2H), 3.80 (s, 1H), 3.71 (d, J=11.5 Hz, 1H), 3.61 (s, 1H), 3.45 (s, 1H), 3.34-3.20 (m, 1H), 1.91 (ddd, J=12.4, 7.8, 3.6 Hz, 1H), 1.86-1.76 (m, 1H), 1.76-1.64 (m, 1H), 1.60 (s, 1H), 1.51 (s, 9H), 1.37 (t, J=7.1 Hz, 3H).

### Methyl N-(2-((S)-3-((tert-butoxycarbonyl)amino)piperidin-1-yl)thiazole-4-carbonyl)-O-(tert-butyldimethylsilyl)-L-serinate (20.2)

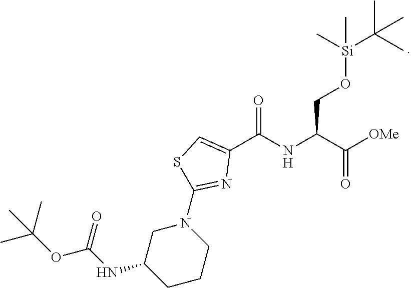 [SMILES: COC(=O)[C@H](CO[Si](C)(C)C(C)(C)C)NC(=O)c1csc(N2CCC[C@H](NC(=O)OC(C)(C)C)C2)n1]

Using the procedure described for Example 22.3, ethyl (S)-2-(3-((tert-butoxycarbonyl)amino)piperidin-1-yl)thiazole-4-carboxylate (0.750 g, 2.11 mmole) was converted to methyl N-(2-((S)-3-((tert-butoxycarbonyl)amino)piperidin-1-yl)thiazole-4-carbonyl)-O-(tert-butyldimethylsilyl)-L-serinate as a colorless oil (1.074 g, 94%). ¹H NMR (400 MHz, CDCl₃): δ 7.94 (d, J=8.8 Hz, 1H), 7.38 (s, 1H), 4.79 (ddd, J=8.8, 3.4, 2.6 Hz, 1H), 4.75-4.65 (m, 1H), 4.19-4.15 (m, 1H), 3.88 (dd, J=10.0, 3.4 Hz, 1H), 3.81 (s, 1H), 3.76 (s, 3H), 3.65 (d, J=12.7 Hz, 1H), 3.55 (d, J=18.5 Hz, 1H), 3.45 (s, 1H), 3.35-3.20 (m, 1H), 1.98-1.76 (m, 2H), 1.71 (dq, J=7.8, 3.8 Hz, 1H), 1.61 (s, 1H), 1.45 (s, 9H), 0.88 (s, 9H), 0.06 (s, 3H), 0.03 (s, 3H).

### Methyl O-acetyl-N—(N-(2-((S)-3-((tert-butoxycarbonyl)amino)piperidin-1-yl)thiazole-4-carbonyl)-O-(tert-butyldimethylsilyl)-L-seryl)-L-serinate (20.3)

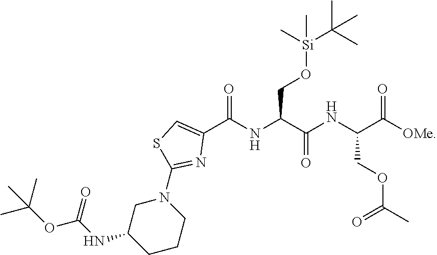 [SMILES: COC(=O)[C@H](COC(C)=O)NC(=O)[C@H](CO[Si](C)(C)C(C)(C)C)NC(=O)c1csc(N2CCC[C@H](NC(=O)OC(C)(C)C)C2)n1]

Using the procedure described in Example 15.6, methyl N-(2-((S)-3-((tert-butoxycarbonyl)amino)piperidin-1-yl)thiazole-4-carbonyl)-O-(tert-butyldimethylsilyl)-L-serinate (1.074 g, 1.98 mmole) was converted to methyl O-acetyl-N—(N-(2-((S)-3-((tert-butoxycarbonyl)amino)piperidin-1-yl)thiazole-4-carbonyl)-O-(tert-butyldimethylsilyl)-L-seryl)-L-serinate as a thick, colorless gel (1.154 g, 87%). ¹H NMR (400 MHz, CDCl₃): δ 7.97 (d, J=7.2 Hz, 1H), 7.43 (d, J=7.7 Hz, 1H), 7.40 (d, J=2.4 Hz, 1H), 4.87 (dt, J=7.7, 3.8 Hz, 1H), 4.69 (s, 1H), 4.59 (td, J=7.1, 3.6 Hz, 1H), 4.52-4.44 (m, 1H), 4.31 (dd, J=11.4, 3.7 Hz, 1H), 4.18 (dd, J=9.8, 3.7 Hz, 1H), 3.76 (d, J=2.8 Hz, 3H), 3.75-3.70 (m, 1H), 3.70-3.52 (m, 2H), 3.44 (s, 1H), 3.36-3.16 (m, 1H), 2.03 (d, J=2.1 Hz, 3H), 1.96-1.86 (m, 1H), 1.87-1.76 (m, 1H), 1.76-1.65 (m, 1H), 1.65-1.59 (m, 1H), 1.45 (s, 9H), 0.92 (s, 9H), 0.14 (s, 3H), 0.13 (s, 3H).

### Methyl (S)-2-(2-(2-(3-((tert-butoxycarbonyl)amino)piperidin-1-yl)thiazole-4-carboxamido)acrylamido)acrylate (20)

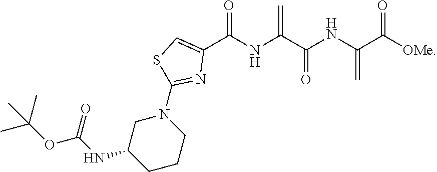 [SMILES: C=C(NC(=O)c1csc(N2CCC[C@H](NC(=O)OC(C)(C)C)C2)n1)C(=O)NC(=C)C(=O)OC]

Using the procedure described for Compound 15, methyl O-acetyl-N—(N-(2-((S)-3-((tert-butoxycarbonyl)amino)piperidin-1-yl)thiazole-4-carbonyl)-O-(tert-butyldimethylsilyl)-L-seryl)-L-serinate (1.154 g, 1.72 mmole) was converted to methyl (S)-2-(2-(2-(3-((tert-butoxycarbonyl)amino)piperidin-1-yl)thiazole-4-carboxamido)acrylamido)acrylate as a white solid (0.372 g, 45%). ¹H NMR (400 MHz, CDCl₃): δ 9.71 (s, 1H), 8.50 (s, 1H), 7.43 (s, 1H), 6.71 (d, J=2.1 Hz, 1H), 6.66 (s, 1H), 6.00 (d, J=1.3 Hz, 1H), 5.43 (t, J=1.8 Hz, 1H), 4.70 (s, 1H), 3.89 (s, 3H), 3.82 (s, 1H), 3.74-3.53 (m, 2H), 3.47 (s, 1H), 3.30 (s, 1H), 1.97-1.78 (m, 2H), 1.72 (qt, J=8.1, 3.7 Hz, 1H), 1.62 (s, 1H), 1.45 (s, 9H).

### Compound 21: Methyl 2-(2-(2-(4-((ethoxycarbonyl)(methyl)amino)piperidin-1-yl)thiazole-4-carboxamido)acrylamido)acrylate

### Tert-butyl 4-((ethoxycarbonyl)(methyl)amino)piperidine-1-carboxylate (21.1)

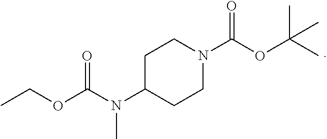 [SMILES: CCOC(=O)N(C)C1CCN(C(=O)OC(C)(C)C)CC1]

Tert-butyl 4-(methylamino)piperidine-1-carboxylate (0.752 g, 3.51 mmole) was dissolved in DCM (7 mL) and cooled to 0° C. N,N-Diisopropylethylamine (0.670 mL, 3.85 mmole) was added followed by dropwise addition of ethyl chloroformate (0.370 mL, 3.87 mmole). The mixture was stirred at 0° for 60 minutes. Water (50 mL) was added and the mixture was extracted with DCM (3×10 mL). The combined organics were dried over magnesium sulfate, filtered, and concentrated. The crude residue was purified by silica gel chromatography (20-60% ethyl acetate/hexane gradient) to provide tert-butyl 4-((ethoxycarbonyl)(methyl)amino)piperidine-1-carboxylate as a colorless oil (0.959 g, 95%). ¹H NMR (400 MHz, CDCl₃): δ 4.27-4.13 (m, 3H), 4.14 (q, J=7.1 Hz, 2H), 2.82-2.69 (m, 2H), 2.76 (s, 3H), 1.60 (qd, J=11.7, 5.8 Hz, 4H), 1.44 (s, 9H), 1.27 (t, J=7.1 Hz, 3H).

### Ethyl 2-(4-((ethoxycarbonyl)(methyl)amino)piperidin-1-yl)thiazole-4-carboxylate (21.2)

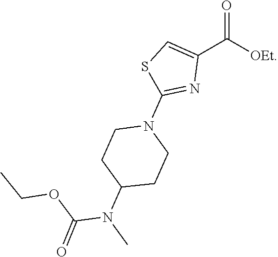 [SMILES: CCOC(=O)c1csc(N2CCC(N(C)C(=O)OCC)CC2)n1]

Tert-butyl 4-((ethoxycarbonyl)(methyl)amino)piperidine-1-carboxylate (0.959 g, 3.35 mmole) was dissolved in DCM (7 mL). HCl (4M solution in 1,4-dioxane, 3.40 mL, 13.6 mmole) was added and the mixture was stirred at RT for 3 hours, then was concentrated. The residue was dissolved in DMA (4 mL). Ethyl 2-bromothiazole-4-carboxylate (0.837 g, 3.55 mmole) was added followed by N,N-diisopropylethylamine (0.940 mL, 5.40 mmole) and the mixture was heated to 80° C. for 18 hours, then was cooled to RT. Ethyl acetate (25 mL) was added and the mixture was washed with water (3×25 mL). The organics were dried over magnesium sulfate, filtered, and concentrated. The crude residue was purified by silica gel chromatography (10-60% ethyl acetate/hexane gradient) to provide ethyl 2-(4-((ethoxycarbonyl)(methyl)amino)piperidin-1-yl)thiazole-4-carboxylate as a yellow oil (0.322 g, 28%). ¹H NMR (400 MHz, CDCl₃): δ 7.44 (s, 1H), 4.35 (q, J=7.1 Hz, 2H), 4.20-4.09 (m, 5H), 3.18-3.05 (m, 2H), 2.77 (s, 3H), 1.78 (qd, J=8.7, 6.8, 3.7 Hz, 4H), 1.37 (t, J=7.1 Hz, 3H), 1.28 (t, J=7.1 Hz, 3H).

### Methyl O-(tert-butyldimethylsilyl)-N-(2-(4-((ethoxycarbonyl)(methyl)amino)piperidin-1-yl)thiazole-4-carbonyl)-L-serinate (21.3)

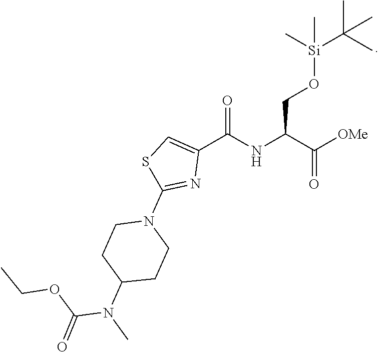 [SMILES: CCOC(=O)N(C)C1CCN(c2nc(C(=O)N[C@@H](CO[Si](C)(C)C(C)(C)C)C(=O)OC)cs2)CC1]

Using the procedure described in Example 19.3, ethyl 2-(4-((ethoxycarbonyl)(methyl)amino)piperidin-1-yl)thiazole-4-carboxylate (0.322 g, 0.943 mmole) was converted to methyl O-(tert-butyldimethylsilyl)-N-(2-(4-((ethoxycarbonyl)(methyl)amino)piperidin-1-yl)thiazole-4-carbonyl)-L-serinate as a thick, colorless oil (0.300 g, 60%). ¹H NMR (400 MHz, CDCl₃): δ 7.95 (d, J=8.8 Hz, 1H), 7.38 (s, 1H), 4.78 (ddd, J=8.8, 3.3, 2.6 Hz, 1H), 4.21-4.02 (m, 6H), 3.89 (dd, J=10.0, 3.4 Hz, 1H), 3.76 (s, 3H), 3.17-3.03 (m, 2H), 2.79 (s, 3H), 1.76 (d, J=4.0 Hz, 4H), 1.33-1.25 (m, 3H), 0.88 (s, 9H), 0.05 (s, 3H), 0.03 (s, 3H).

### Methyl O-acetyl-N—(O-(tert-butyldimethylsilyl)-N-(2-(4-((ethoxycarbonyl)(methyl)amino)piperidin-1-yl)thiazole-4-carbonyl)-L-seryl)-L-serinate (21.4)

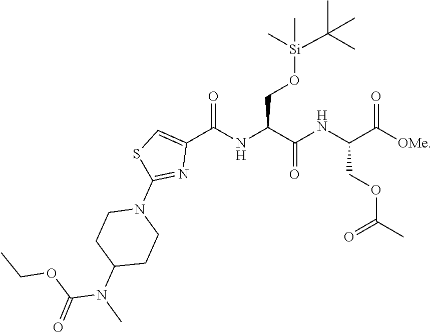 [SMILES: CCOC(=O)N(C)C1CCN(c2nc(C(=O)N[C@@H](CO[Si](C)(C)C(C)(C)C)C(=O)N[C@@H](COC(C)=O)C(=O)OC)cs2)CC1]

Using the procedure described in Example 15.6, methyl O-(tert-butyldimethylsilyl)-N-(2-(4-((ethoxycarbonyl)(methyl)amino)piperidin-1-yl)thiazole-4-carbonyl)-L-serinate (0.300 g, 0.567 mmole) was converted to methyl O-acetyl-N—(O-(tert-butyldimethylsilyl)-N-(2-(4-((ethoxycarbonyl)(methyl)amino)piperidin-1-yl)thiazole-4-carbonyl)-L-seryl)-L-serinate as a white solid (0.297 g, 80%). ¹H NMR (400 MHz, CDCl₃): δ 7.98 (d, J=7.1 Hz, 1H), 7.43 (d, J=7.5 Hz, 1H), 7.39 (d, J=2.5 Hz, 1H), 4.87 (dt, J=7.7, 3.8 Hz, 1H), 4.58 (td, J=7.2, 3.6 Hz, 1H), 4.51-4.45 (m, 1H), 4.31 (dd, J=11.4, 3.7 Hz, 1H), 4.22-4.14 (m, 3H), 4.14-4.04 (m, 3H), 3.76 (d, J=2.2 Hz, 3H), 3.75-3.70 (m, 1H), 3.11 (d, J=12.5 Hz, 2H), 2.78 (s, 3H), 2.02 (d, J=2.7 Hz, 3H), 1.77 (s, 4H), 1.29 (t, J=7.7 Hz, 3H), 0.92 (s, 9H), 0.14 (s, 3H), 0.13 (s, 3H).

### Methyl 2-(2-(2-(4-((ethoxycarbonyl)(methyl)amino)piperidin-1-yl)thiazole-4-carboxamido)acrylamido)acrylate (21)

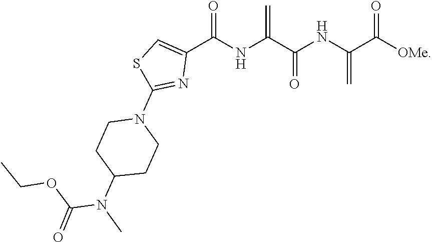 [SMILES: C=C(NC(=O)c1csc(N2CCC(N(C)C(=O)OCC)CC2)n1)C(=O)NC(=C)C(=O)OC]

Using the procedure described for Compound 15, methyl O-acetyl-N—(O-(tert-butyldimethylsilyl)-N-(2-(4-((ethoxycarbonyl)(methyl)amino)piperidin-1-yl)thiazole-4-carbonyl)-L-seryl)-L-serinate (0.297 g, 0.451 mmole) was converted to methyl 2-(2-(2-(4-((ethoxycarbonyl)(methyl)amino)piperidin-1-yl)thiazole-4-carboxamido)acrylamido)acrylate as a white solid (0.090 g, 43%). ¹H NMR (400 MHz, CDCl₃): δ 9.72 (s, 1H), 8.51 (s, 1H), 7.42 (s, 1H), 6.72 (d, J=2.1 Hz, 1H), 6.66 (s, 1H), 6.00 (d, J=1.3 Hz, 1H), 5.44 (t, J=1.8 Hz, 1H), 4.28 (s, 1H), 4.20-4.08 (m, 4H), 3.89 (s, 3H), 3.18-3.04 (m, 2H), 2.79 (s, 3H), 1.79 (s, 4H), 1.28 (t, J=7.1 Hz, 3H).

### Compound 22: Methyl 2-(2-(2-(4-(((ethoxycarbonyl)amino)methyl)piperidin-1-yl)thiazole-4-carboxamido)acrylamido)acrylate

### Tert-butyl 4-(((ethoxycarbonyl)amino)methyl)piperidine-1-carboxylate (22.1)

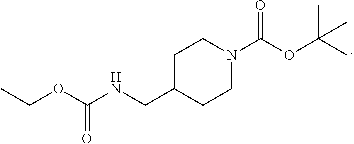 [SMILES: CCOC(=O)NCC1CCN(C(=O)OC(C)(C)C)CC1]

Tert-butyl 4-(aminomethyl)piperidine-1-carboxylate (0.754 g, 3.52 mmole) was dissolved in DCM (7 mL) and cooled to 0° C. N,N-Diisopropylethylamine (0.730 mL, 4.19 mmole) was added followed by dropwise addition of ethyl chloroformate (0.370 mL, 3.87 mmole). The mixture was stirred at 0° for 60 minutes. Water (25 mL) was added and the two layers were separated. The aqueous layer was extracted with DCM (2×10 mL). The combined organics were dried over magnesium sulfate, filtered, and concentrated. The crude residue was purified by silica gel chromatography (20-40% ethyl acetate/hexane gradient) to provide tert-butyl 4-(((ethoxycarbonyl)amino)methyl)piperidine-1-carboxylate as a thick, colorless oil (0.997 g, 99%). ¹H NMR (400 MHz, CDCl₃): δ 4.77 (s, 1H), 4.19-4.03 (m, 4H), 3.07 (t, J=6.4 Hz, 2H), 2.68 (t, J=12.9 Hz, 2H), 1.71-1.63 (m, 2H), 1.44 (s, 9H), 1.25 (dt, J=9.0, 7.1 Hz, 4H), 1.17-1.03 (m, 2H).

### Ethyl 2-(4-(((ethoxycarbonyl)amino)methyl)piperidin-1-yl)thiazole-4-carboxylate (22.2)

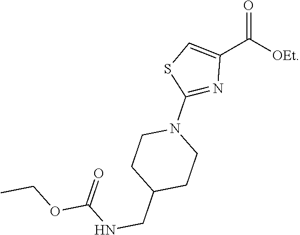 [SMILES: CCOC(=O)NCC1CCN(c2nc(C(=O)OCC)cs2)CC1]

Tert-butyl 4-(((ethoxycarbonyl)amino)methyl)piperidine-1-carboxylate (0.997 g, 3.48 mmole) was dissolved in DCM (7 mL). HCl (4M solution in 1,4-dioxane, 3.50 mL, 14.0 mmole) was added and the mixture was stirred at RT for 3 hours, then was concentrated. The residue was dissolved in DMA (4 mL). Ethyl 2-bromothiazole-4-carboxylate (0.905 g, 3.83 mmole) and N,N-diisopropylethylamine (1.20 mL, 6.89 mmole) were added and the mixture was heated to 80° C. for 18 hours and was cooled to RT, then diluted with ethyl acetate (25 mL). The mixture was washed with water (3×25 mL) and the organics were dried over magnesium sulfate, filtered, and concentrated. The crude residue was purified by silica gel chromatography (10-70% ethyl acetate/hexane gradient) to provide ethyl 2-(4-(((ethoxycarbonyl)amino)methyl)piperidin-1-yl)thiazole-4-carboxylate as a yellow solid (0.600 g, 50%). ¹H NMR (400 MHz, CDCl₃): δ 7.42 (s, 1H), 4.77 (s, 1H), 4.35 (q, J=7.1 Hz, 2H), 4.18-4.02 (m, 4H), 3.11 (t, J=6.4 Hz, 2H), 3.01 (td, J=12.7, 2.8 Hz, 2H), 1.86-1.77 (m, 2H), 1.73 (d, J=6.8 Hz, 1H), 1.37 (t, J=7.1 Hz, 3H), 1.35-1.29 (m, 2H), 1.25 (td, J=7.1, 5.9 Hz, 3H).

### Methyl O-(tert-butyldimethylsilyl)-N-(2-(4-(((ethoxycarbonyl)amino)methyl)piperidin-1-yl)thiazole-4-carbonyl)-L-serinate (22.3)

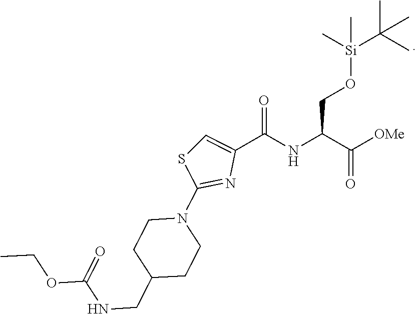 [SMILES: CCOC(=O)NCC1CCN(c2nc(C(=O)N[C@@H](CO[Si](C)(C)C(C)(C)C)C(=O)OC)cs2)CC1]

Using the procedure described in Example 19.3, ethyl 2-(4-(((ethoxycarbonyl)amino)methyl)piperidin-1-yl)thiazole-4-carboxylate (0.600 mL, 1.76 mmole) was converted to methyl O-(tert-butyldimethylsilyl)-N-(2-(4-(((ethoxycarbonyl)amino)methyl)piperidin-1-yl)thiazole-4-carbonyl)-L-serinate as a thick, colorless gel (0.897 g, 96%). ¹H NMR (400 MHz, CDCl₃): δ 7.96 (d, J=8.8 Hz, 1H), 7.36 (s, 1H), 4.78 (dt, J=8.8, 3.0 Hz, 2H), 4.20-4.08 (m, 3H), 4.02 (dd, J=11.7, 5.3 Hz, 2H), 3.88 (dd, J=10.0, 3.4 Hz, 1H), 3.76 (s, 3H), 3.12 (t, J=6.4 Hz, 2H), 2.99 (tdd, J=12.5, 6.0, 2.6 Hz, 2H), 1.81 (s, 2H), 1.34 (tt, J=11.5, 5.2 Hz, 2H), 1.26 (td, J=7.1, 2.9 Hz, 3H), 0.88 (s, 9H), 0.05 (s, 3H), 0.03 (s, 3H).

### Methyl O-acetyl-N—(O-(tert-butyldimethylsilyl)-N-(2-(4-(((ethoxycarbonyl)amino)methyl)piperidin-1-yl)thiazole-4-carbonyl)-L-seryl)-L-serinate (22.4)

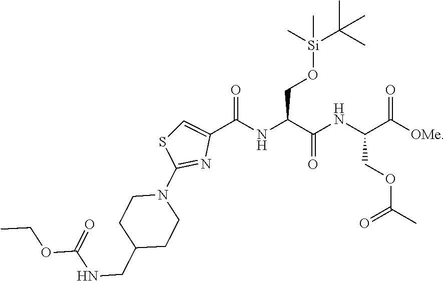 [SMILES: CCOC(=O)NCC1CCN(c2nc(C(=O)N[C@@H](CO[Si](C)(C)C(C)(C)C)C(=O)N[C@@H](COC(C)=O)C(=O)OC)cs2)CC1]

Using the procedure described in Example 15.6, methyl O-(tert-butyldimethylsilyl)-N-(2-(4-(((ethoxycarbonyl)amino)methyl)piperidin-1-yl)thiazole-4-carbonyl)-L-serinate (0.897 g, 1.70 mmole) was converted to methyl O-acetyl-N—(O-(tert-butyldimethylsilyl)-N-(2-(4-(((ethoxycarbonyl)amino)methyl)piperidin-1-yl)thiazole-4-carbonyl)-L-seryl)-L-serinate as a colorless oil (0.921 g, 82%). ¹H NMR (400 MHz, CDCl₃): δ 7.99 (d, J=7.1 Hz, 1H), 7.43 (d, J=7.8 Hz, 1H), 7.37 (d, J=2.8 Hz, 1H), 4.87 (dq, J=8.0, 3.9 Hz, 1H), 4.77 (s, 1H), 4.58 (ddd, J=7.2, 5.9, 3.6 Hz, 1H), 4.47 (dt, J=11.3, 4.2 Hz, 1H), 4.31 (dd, J=11.4, 3.7 Hz, 1H), 4.19 (ddd, J=9.8, 3.6, 2.0 Hz, 1H), 4.12 (q, J=7.1 Hz, 2H), 4.01 (d, J=13.0 Hz, 2H), 3.76 (d, J=2.3 Hz, 3H), 3.75-3.70 (m, 1H), 3.12 (t, J=6.4 Hz, 2H), 2.99 (tt, J=12.8, 3.0 Hz, 2H), 2.02 (d, J=2.4 Hz, 3H), 1.82 (s, 2H), 1.32 (d, J=12.2 Hz, 2H), 1.26 (td, J=7.1, 3.5 Hz, 3H), 0.92 (s, 9H), 0.14 (s, 3H), 0.12 (s, 3H).

### Methyl 2-(2-(2-(4-(((ethoxycarbonyl)amino)methyl)piperidin-1-yl)thiazole-4-carboxamido)acrylamido)acrylate (22)

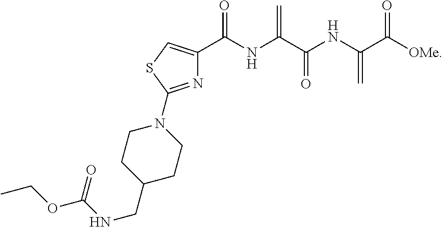 [SMILES: C=C(NC(=O)c1csc(N2CCC(CNC(=O)OCC)CC2)n1)C(=O)NC(=C)C(=O)OC]

Using the procedure as described for Compound 15, methyl O-acetyl-N—(O-(tert-butyldimethylsilyl)-N-(2-(4-(((ethoxycarbonyl)amino)methyl)piperidin-1-yl)thiazole-4-carbonyl)-L-seryl)-L-serinate (0.921 g, 1.40 mmole) was converted to methyl 2-(2-(2-(4-(((ethoxycarbonyl)amino)methyl)piperidin-1-yl)thiazole-4-carboxamido)acrylamido)acrylate as a white solid (0.310 g, 48%). ¹H NMR (400 MHz, CDCl₃): δ 9.78-9.67 (m, 1H), 8.51 (s, 1H), 7.41 (s, 1H), 6.71 (d, J=2.1 Hz, 1H), 6.66 (s, 1H), 6.00 (d, J=1.3 Hz, 1H), 5.43 (t, J=1.9 Hz, 1H), 4.76 (s, 1H), 4.12 (q, J=7.1 Hz, 2H), 4.09-3.98 (m, 2H), 3.89 (s, 3H), 3.12 (t, J=6.4 Hz, 2H), 3.02 (td, J=12.7, 2.8 Hz, 2H), 1.85 (d, J=3.5 Hz, 2H), 1.35 (td, J=12.3, 4.4 Hz, 2H), 1.25 (t, J=7.1 Hz, 3H).

### Compound 23: Methyl (R)-2-(2-(2-(3-((ethoxycarbonyl)amino)pyrrolidin-1-yl)thiazole-4-carboxamido)acrylamido)acrylate

### Tert-butyl (R)-3-((ethoxycarbonyl)amino)pyrrolidine-1-carboxylate (23.1)

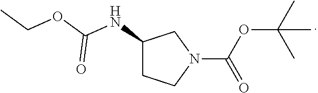 [SMILES: CCOC(=O)N[C@@H]1CCN(C(=O)OC(C)(C)C)C1]

Tert-butyl (R)-3-aminopyrrolidine-1-carboxylate (1.016 g, 5.45 mmole) was dissolved in DCM (11 mL) and cooled to 0° C. N,N-Diisopropylethylamine (1.15 mL, 6.60 mmole) was added followed by ethyl chloroformate (0.560 mL, 5.86 mmole). The mixture was stirred at 0° for 90 minutes and water (50 mL) was added. The two layers were separated and the aqueous layer was extracted with DCM (2×15 mL). The combined organics were dried over magnesium sulfate, filtered, and concentrated. The crude residue was purified by silica gel chromatography (30-60% ethyl acetate/hexane) to provide tert-butyl (R)-3-((ethoxycarbonyl)amino)pyrrolidine-1-carboxylate as a colorless oil (1.597 g). ¹H NMR (400 MHz, CDCl₃): δ 4.84 (s, 1H), 4.23 (d, J=8.2 Hz, 1H), 4.12 (q, J=7.1 Hz, 2H), 3.60 (dd, J=11.4, 6.2 Hz, 1H), 3.50-3.34 (m, 2H), 3.20 (d, J=25.0 Hz, 1H), 2.13 (dtd, J=13.2, 7.5, 5.9 Hz, 1H), 1.90-1.75 (m, 1H), 1.43 (s, 9H), 1.25 (td, J=7.2, 5.6 Hz, 3H).

### Ethyl (R)-2-(3-((ethoxycarbonyl)amino)pyrrolidin-1-yl)thiazole-4-carboxylate (23.2)

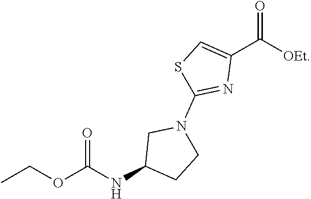 [SMILES: CCOC(=O)N[C@@H]1CCN(c2nc(C(=O)OCC)cs2)C1]

Tert-butyl (R)-3-((ethoxycarbonyl)amino)pyrrolidine-1-carboxylate (1.597 g, 6.18 mmole) was dissolved in DCM (12 mL). HCl (4M solution in 1,4-dioxane, 6.20 mL, 24.8 mmole) was added and the mixture was stirred at RT for 75 minutes and was concentrated. The residue was dissolved in DMA (6 mL) and ethyl 2-bromothiazole-4-carboxylate (1.613 g, 6.83 mmole) and N,N-diisopropylethylamine (2.15 mL, 12.3 mmole) were added. The mixture was heated to 80° C. for 3 days and was cooled to RT. Ethyl acetate (25 mL) was added and the mixture was washed with water (3×25 mL). The organics were dried over magnesium sulfate, filtered, and concentrated. The residue was purified by silica gel chromatography (30-70% ethyl acetate/hexane gradient) to provide ethyl (R)-2-(3-((ethoxycarbonyl)amino)pyrrolidin-1-yl)thiazole-4-carboxylate as a white solid (1.105 g, 57%). ¹H NMR (400 MHz, CDCl₃): δ 7.40 (s, 1H), 4.83 (s, 1H), 4.47-4.39 (m, 1H), 4.36 (q, J=7.1 Hz, 2H), 4.13 (q, J=7.0 Hz, 2H), 3.79 (dd, J=10.7, 6.0 Hz, 1H), 3.61 (qt, J=10.0, 6.4 Hz, 2H), 3.43 (dd, J=10.6, 4.2 Hz, 1H), 2.33 (dddd, J=13.0, 8.1, 7.2, 6.0 Hz, 1H), 2.09-1.96 (m, 1H), 1.37 (t, J=7.1 Hz, 3H), 1.25 (t, J=7.1 Hz, 3H).

### Methyl O-(tert-butyldimethylsilyl)-N-(2-((R)-3-((ethoxycarbonyl)amino)pyrrolidin-1-yl)thiazole-4-carbonyl)-L-serinate (23.3)

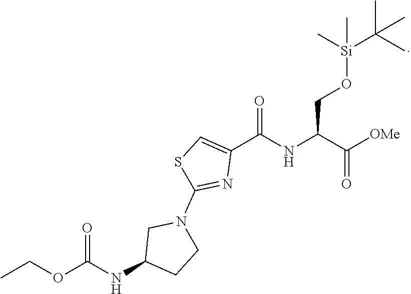 [SMILES: CCOC(=O)N[C@@H]1CCN(c2nc(C(=O)N[C@@H](CO[Si](C)(C)C(C)(C)C)C(=O)OC)cs2)C1]

Using the procedure described in Example 19.3, ethyl (R)-2-(3-((ethoxycarbonyl)amino)pyrrolidin-1-yl)thiazole-4-carboxylate (1.105 g, 3.53 mmole) was converted to methyl O-(tert-butyldimethylsilyl)-N-(2-((R)-3-((ethoxycarbonyl)amino)pyrrolidin-1-yl)thiazole-4-carbonyl)-L-serinate as a colorless oil (0.859 g, 49%). ¹H NMR (400 MHz, CDCl₃): δ 7.99 (d, J=8.8 Hz, 1H), 7.34 (s, 1H), 4.90 (s, 1H), 4.84-4.75 (m, 1H), 4.43 (s, 1H), 4.19-4.12 (m, 3H), 3.89 (dd, J=10.0, 3.4 Hz, 1H), 3.76 (s, 3H), 3.75-3.69 (m, 1H), 3.59-3.51 (m, 2H), 3.38 (dd, J=10.8, 4.2 Hz, 1H), 2.41-2.27 (m, 1H), 2.07-1.98 (m, 1H), 1.26 (t, J=7.1 Hz, 3H), 0.89 (s, 9H), 0.06 (s, 3H), 0.03 (s, 3H).

### Methyl O-acetyl-N—(O-(tert-butyldimethylsilyl)-N-(2-((R)-3-((ethoxycarbonyl)amino)pyrrolidin-1-yl)thiazole-4-carbonyl)-L-seryl)-L-serinate (23.4)

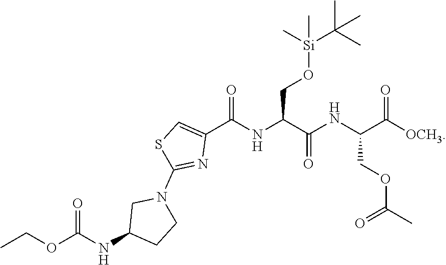 [SMILES: CCOC(=O)N[C@@H]1CCN(c2nc(C(=O)N[C@@H](CO[Si](C)(C)C(C)(C)C)C(=O)N[C@@H](COC(C)=O)C(=O)OC)cs2)C1]

Using the procedure described in Example 15.6, methyl O-(tert-butyldimethylsilyl)-N-(2-((R)-3-((ethoxycarbonyl)amino)pyrrolidin-1-yl)thiazole-4-carbonyl)-L-serinate (0.859 g, 1.72 mmole) was converted to methyl O-acetyl-N—(O-(tert-butyldimethylsilyl)-N-(2-((R)-3-((ethoxycarbonyl)amino)pyrrolidin-1-yl)thiazole-4-carbonyl)-L-seryl)-L-serinate as a pale yellow oil (0.753 g, 70%). ¹H NMR (400 MHz, CDCl₃): δ 8.02 (d, J=7.2 Hz, 1H), 7.44 (d, J=7.8 Hz, 1H), 7.35 (d, J=2.4 Hz, 1H), 4.87 (dq, J=7.7, 3.9 Hz, 2H), 4.59 (ddd, J=7.2, 6.0, 3.7 Hz, 1H), 4.51-4.44 (m, 1H), 4.43 (d, J=7.5 Hz, 1H), 4.31 (dd, J=11.4, 3.7 Hz, 1H), 4.18 (dd, J=9.8, 3.7 Hz, 1H), 4.12 (q, J=7.2 Hz, 3H), 3.76 (d, J=2.5 Hz, 4H), 3.75-3.70 (m, 1H), 3.56 (ddt, J=7.8, 5.8, 2.7 Hz, 2H), 3.38 (dd, J=10.7, 4.2 Hz, 1H), 2.41-2.27 (m, 1H), 2.06-1.99 (m, 1H), 2.02 (s, 3H), 1.26 (td, J=7.1, 2.2 Hz, 3H), 0.93 (d, J=1.4 Hz, 9H), 0.14 (s, 3H), 0.13 (s, 3H).

### Methyl (R)-2-(2-(2-(3-((ethoxycarbonyl)amino)pyrrolidin-1-yl)thiazole-4-carboxamido)acrylamido)acrylate (23)

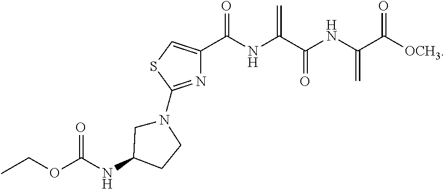 [SMILES: C=C(NC(=O)c1csc(N2CC[C@@H](NC(=O)OCC)C2)n1)C(=O)NC(=C)C(=O)OC]

Using the procedure as described for Compound 15, methyl O-acetyl-N—(O-(tert-butyldimethylsilyl)-N-(2-((R)-3-((ethoxycarbonyl)amino)pyrrolidin-1-yl)thiazole-4-carbonyl)-L-seryl)-L-serinate (0.753 g, 1.20 mmole) was converted to methyl (R)-2-(2-(2-(3-((ethoxycarbonyl)amino)pyrrolidin-1-yl)thiazole-4-carboxamido)acrylamido)acrylate as a white solid (0.224 g, 43%). ¹H NMR (400 MHz, CDCl₃): δ 9.77 (s, 1H), 8.50 (s, 1H), 7.38 (s, 1H), 6.71 (d, J=2.1 Hz, 1H), 6.67 (s, 1H), 6.00 (d, J=1.3 Hz, 1H), 5.43 (t, J=1.9 Hz, 1H), 4.85 (s, 1H), 4.44 (s, 1H), 4.13 (p, J=7.0 Hz, 2H), 3.89 (s, 3H), 3.79 (dd, J=10.7, 6.1 Hz, 1H), 3.69-3.52 (m, 2H), 3.41 (dd, J=10.6, 4.3 Hz, 1H), 2.35 (dddd, J=12.9, 8.1, 7.1, 6.0 Hz, 1H), 2.04 (d, J=5.0 Hz, 1H), 1.32-1.20 (m, 3H).

### Compound 24: Methyl 2-(2-(2-(4-(2-methoxyethoxy)phenyl)thiazole-4-carboxamido)acrylamido)acrylate

### 4-(2-Methoxyethoxy)benzonitrile (24.1)

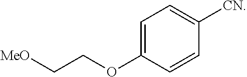 [SMILES: COCCOc1ccc(C#N)cc1]

2-Methoxyethanol (1.008 g, 13.2 mmole) was dissolved in DMF (13 mL). Sodium hydride (60% dispersion in mineral oil, 0.578 g, 14.5 mmole) was added portionwise (gas evolution was observed). The mixture was stirred at RT for 30 minutes and 4-fluorobenzonitrile (1.670 g, 13.8 mmole) was added. The mixture was heated to 80° C. for 2 ½ hours and was cooled to RT. Ethyl acetate (25 mL) was added and the mixture was washed with water (3×25 mL). The organics were dried over magnesium sulfate, filtered, and concentrated. The crude residue was purified by silica gel chromatography (5-55% ethyl acetate/hexane gradient) to provide 4-(2-methoxyethoxy)benzonitrile as a colorless solid (1.406 g, 60%). ¹H NMR (400 MHz, CDCl₃): δ 7.62-7.55 (m, 2H), 7.02-6.94 (m, 2H), 4.20-4.13 (m, 2H), 3.80-3.73 (m, 2H), 3.45 (s, 3H).

### 4-(2-Methoxyethoxy)benzothioamide (24.2)

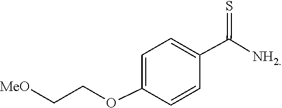 [SMILES: COCCOc1ccc(C(N)=S)cc1]

4-(2-Methoxyethoxy)benzonitrile (1.406 g, 7.93 mmole) was dissolved in pyridine (8 mL). Triethylamine (1.20 mL, 8.56 mmole) was added followed by ammonium sulfide (40% aqueous solution, 1.60 mL, 9.36 mmole). The mixture was heated to 50° C. for 20 hours and was cooled to RT, then was concentrated. The residue was treated with diethyl ether (20 mL) and the solid was collected by filtration to provide 4-(2-methoxyethoxy)benzothioamide as a yellow solid (1.070 g, 64%). ¹H NMR (400 MHz, DMSO-d₆): δ 9.64 (s, 1H), 9.32 (s, 1H), 8.01-7.89 (m, 2H), 7.01-6.91 (m, 2H), 4.21-4.11 (m, 2H), 3.72-3.62 (m, 2H), 3.31 (s, 3H).

### Ethyl 2-(4-(2-methoxyethoxy)phenyl)thiazole-4-carboxylate (24.3)

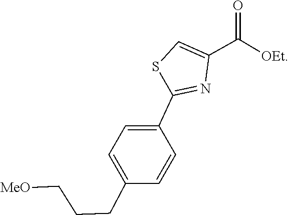 [SMILES: CCOC(=O)c1csc(-c2ccc(CCCOC)cc2)n1]

4-(2-Methoxyethoxy)benzothioamide (1.070 g, 5.06 mmole) was dissolved in ethanol (10 mL). Ethyl bromopyruvate (0.770 mL, 6.14 mmole) was added and the mixture was heated to 80° C. for 3 hours, then was cooled to RT, causing a solid to precipitate from solution. The solid was collected by filtration, washed with cold ethanol, and allowed to air dry to provide ethyl 2-(4-(2-methoxyethoxy)phenyl)thiazole-4-carboxylate as a yellow solid (0.931 g, 60%). ¹H NMR (400 MHz, CDCl₃): δ 8.09 (s, 1H), 7.98-7.90 (m, 2H), 7.02-6.94 (m, 2H), 4.44 (q, J=7.1 Hz, 2H), 4.22-4.14 (m, 2H), 3.82-3.74 (m, 2H), 3.46 (s, 3H), 1.43 (t, J=7.1 Hz, 3H).

### Methyl O-(tert-butyldimethylsilyl)-N-(2-(4-(2-methoxyethoxy)phenyl)thiazole-4-carbonyl)-L-serinate (24.4)

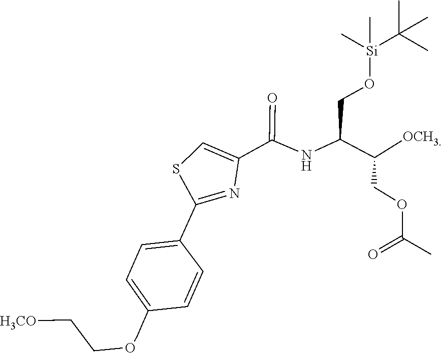 [SMILES: COCCOCc1ccc(-c2nc(C(=O)N[C@@H](CO[Si](C)(C)C(C)(C)C)[C@@H](COC(C)=O)OC)cs2)cc1]

Using the procedure described in Example 19.3, ethyl 2-(4-(2-methoxyethoxy)phenyl)thiazole-4-carboxylate (0.931 g, 3.03 mmole) was converted to methyl O-(tert-butyldimethylsilyl)-N-(2-(4-(2-methoxyethoxy)phenyl)thiazole-4-carbonyl)-L-serinate as a colorless oil (1.481 g, 99%). ¹H NMR (400 MHz, CDCl₃): δ 8.23 (d, J=8.7 Hz, 1H), 8.03 (s, 1H), 7.94-7.85 (m, 2H), 7.04-6.95 (m, 2H), 4.85 (dt, J=8.7, 3.1 Hz, 1H), 4.24-4.16 (m, 3H), 3.95 (dd, J=10.0, 3.4 Hz, 1H), 3.83-3.74 (m, 2H), 3.74 (s, 3H), 3.47 (s, 3H), 0.92 (s, 9H), 0.07 (s, 3H), 0.06 (s, 3H).

### Methyl O-acetyl-N—(O-(tert-butyldimethylsilyl)-N-(2-(4-(2-methoxyethoxy)phenyl)thiazole-4-carbonyl)-L-seryl)-L-serinate (24.5)

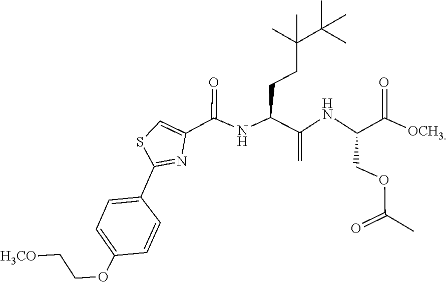 [SMILES: C=C(N[C@@H](COC(C)=O)C(=O)OC)[C@H](CCC(C)(C)C(C)(C)C)NC(=O)c1csc(-c2ccc(OCCOC)cc2)n1]

Using the procedure described in Example 15.6, methyl O-(tert-butyldimethylsilyl)-N-(2-(4-(2-methoxyethoxy)phenyl)thiazole-4-carbonyl)-L-serinate (0.701 g, 1.42 mmole) was converted to methyl O-acetyl-N—(O-(tert-butyldimethylsilyl)-N-(2-(4-(2-methoxyethoxy)phenyl)thiazole-4-carbonyl)-L-seryl)-L-serinate as a colorless oil (0.644 g, 73%). ¹H NMR (400 MHz, CDCl₃): δ 8.27 (d, J=7.3 Hz, 1H), 8.04 (d, J=2.8 Hz, 1H), 7.95-7.85 (m, 2H), 7.46 (d, J=7.8 Hz, 1H), 6.99 (dd, J=8.3, 1.5 Hz, 2H), 4.89 (tt, J=6.5, 3.8 Hz, 1H), 4.66 (ddd, J=8.9, 6.5, 3.7 Hz, 1H), 4.54-4.46 (m, 1H), 4.33 (dd, J=11.4, 3.6 Hz, 1H), 4.23 (dd, J=9.8, 3.7 Hz, 1H), 4.21-4.17 (m, 2H), 3.82-3.78 (m, 2H), 3.78-3.74 (m, 1H), 3.76 (s, 3H), 3.47 (s, 3H), 2.01 (s, 3H), 0.95 (s, 9H), 0.15 (s, 3H), 0.14 (s, 3H).

### Methyl 2-(2-(2-(4-(2-methoxyethoxy)phenyl)thiazole-4-carboxamido)acrylamido)acrylate (24)

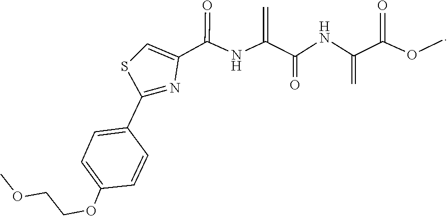

Using the procedure as described for Compound 15, methyl O-acetyl-N—(O-(tert-butyldimethylsilyl)-N-(2-(4-(2-methoxyethoxy)phenyl)thiazole-4-carbonyl)-L-seryl)-L-serinate (0.644 g, 1.03 mmole) was converted to methyl 2-(2-(2-(4-(2-methoxyethoxy)phenyl)thiazole-4-carboxamido)acrylamido)acrylate as a white solid (0.203 g, 46%). ¹H NMR (400 MHz, CDCl₃): δ 10.01 (s, 1H), 8.55 (s, 1H), 8.08 (s, 1H), 7.99-7.90 (m, 2H), 7.06-6.96 (m, 2H), 6.78 (d, J=2.2 Hz, 1H), 6.71 (s, 1H), 6.03 (d, J=1.3 Hz, 1H), 5.49 (t, J=1.9 Hz, 1H), 4.25-4.13 (m, 2H), 3.90 (s, 3H), 3.85-3.74 (m, 2H), 3.48 (s, 3H).

### Compound 25: Methyl 2-(2-(2-(4-(6-azidohexanamido)phenyl)thiazole-4-carboxamido)acrylamido)acrylate

### Tert-butyl (4-carbamothioylphenyl)carbamate (25.1)

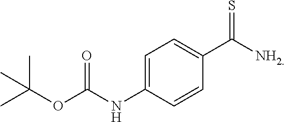 [SMILES: CC(C)(C)OC(=O)Nc1ccc(C(N)=S)cc1]

Tert-butyl (4-cyanophenyl)carbamate (1.002 g, 4.59 mmole) was dissolved in pyridine (5 mL). Triethylamine (0.710 mL, 5.07 mmole) and ammonium sulfide (40% aqueous solution, 0.940 mL, 5.50 mmole) were added and the mixture was heated to 50° C. for 18 hours, then was cooled to RT and concentrated. The residue was suspended in ethyl acetate (25 mL) and the mixture was washed with 1N aq. HCl (2×25 mL). The organics were dried over magnesium sulfate, filtered, and concentrated to provide tert-butyl (4-carbamothioylphenyl)carbamate as a yellow solid (1.102 g, 95%). ¹H NMR (400 MHz, DMSO-d₆): δ 9.64 (d, J=4.7 Hz, 2H), 9.30 (s, 1H), 7.92-7.83 (m, 2H), 7.52-7.42 (m, 2H), 1.48 (s, 9H).

### Ethyl 2-(4-(6-bromohexanamido)phenyl)thiazole-4-carboxylate (25.2)

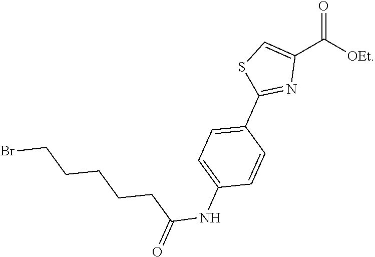 [SMILES: CCOC(=O)c1csc(-c2ccc(NC(=O)CCCCCBr)cc2)n1]

Tert-butyl (4-carbamothioylphenyl)carbamate (1.102 g, 4.37 mmole) was dissolved in ethanol (9 mL). Ethyl bromopyruvate (0.610 mL, 4.86 mmole) was added and the mixture was heated to 80° C. for 2 hours then was cooled to RT and concentrated to provide ethyl 2-(4-aminophenyl)thiazole-4-carboxylate hydrobromide as an orange solid (1.591 g). 6-Bromohexanoic acid (1.373 g, 7.04 mmole) was dissolved in DCM (13 mL). DMF (2 drops) was added followed by slow addition of oxalyl chloride (0.620 mL, 7.11 mmole; gas evolution was observed). The solution was stirred at RT for 2 hours and was concentrated. The residue was dissolved in DCM (2 mL) and added slowly to a cold (0° C.) mixture of ethyl 2-(4-aminophenyl)thiazole-4-carboxylate hydrobromide (1.591 g) and N,N-diisopropylethylamine (2.25 mL, 12.9 mmole) in DCM (13 mL). The resulting mixture was stirred at 0° for 90 minutes and was poured into 1N aq. HCl (25 mL) and water (25 mL). The two layers were separated and the aqueous layer was extracted with DCM (2×15 mL). The combined organics were dried over magnesium sulfate, filtered, and concentrated. The crude residue was purified by silica gel chromatography (10-70% ethyl acetate/hexane gradient) to provide ethyl 2-(4-(6-bromohexanamido)phenyl)thiazole-4-carboxylate as a yellow solid (1.211 g, 65%). ¹H NMR (400 MHz, CDCl₃): δ 8.12 (s, 1H), 8.00-7.91 (m, 2H), 7.68 (d, J=8.7 Hz, 2H), 7.64 (s, 1H), 4.45 (q, J=7.1 Hz, 2H), 3.42 (t, J=6.7 Hz, 2H), 2.42 (t, J=7.4 Hz, 2H), 1.90 (dq, J=10.4, 6.8 Hz, 2H), 1.77 (tt, J=8.1, 6.3 Hz, 2H), 1.53 (ddt, J=12.2, 6.5, 3.9 Hz, 2H), 1.43 (t, J=7.1 Hz, 3H).

### Ethyl 2-(4-(6-azidohexanamido)phenyl)thiazole-4-carboxylate (25.3)

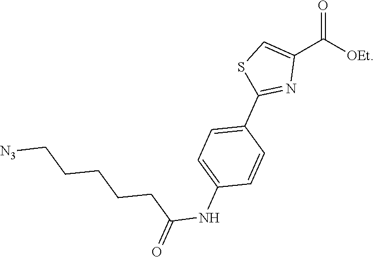 [SMILES: CCOC(=O)c1csc(-c2ccc(NC(=O)CCCCCN=[N+]=[N-])cc2)n1]

Ethyl 2-(4-(6-bromo exanamio)penytiazo e-4-car oxy late (1.211 g, 2.85 mmole) was dissolved in DMF (6 mL). Sodium azide (0.224 g, 3.45 mmole) was added and the mixture was heated to 80° C. for 3 hours. The mixture was cooled to RT and diluted with ethyl acetate (25 mL). The mixture was washed with water (3×25 mL) and sat. aq. sodium chloride (1×25 mL), then dried over magnesium sulfate, filtered, and concentrated to provide ethyl 2-(4-(6-azidohexanamido)phenyl)thiazole-4-carboxylate as a yellow solid (0.899 g, 81%). ¹H NMR (400 MHz, CDCl₃): δ 8.12 (s, 1H), 8.00-7.93 (m, 2H), 7.66 (d, J=8.6 Hz, 2H), 7.55 (s, 1H), 4.45 (q, J=7.1 Hz, 2H), 3.29 (t, J=6.8 Hz, 2H), 2.41 (t, J=7.4 Hz, 2H), 1.77 (tt, J=8.1, 6.4 Hz, 2H), 1.71-1.58 (m, 2H), 1.51-1.46 (m, 2H), 1.43 (t, J=7.1 Hz, 3H).

### Methyl N-(2-(4-(6-azidohexanamido)phenyl)thiazole-4-carbonyl)-O-(tert-butyldimethylsilyl)-L-serinate (25.4)

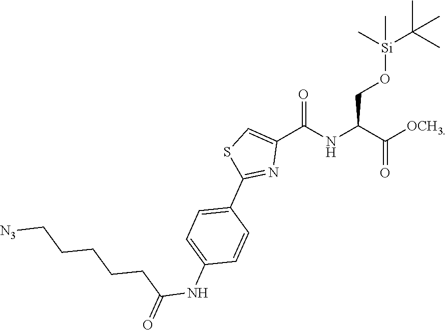 [SMILES: COC(=O)[C@H](CO[Si](C)(C)C(C)(C)C)NC(=O)c1csc(-c2ccc(NC(=O)CCCCCN=[N+]=[N-])cc2)n1]

Using the procedure described for Example 19.3, ethyl 2-(4-(6-azidohexanamido)phenyl)thiazole-4-carboxylate (0.899 g, 2.32 mmole) was converted to methyl N-(2-(4-(6-azidohexanamido)phenyl)thiazole-4-carbonyl)-O-(tert-butyldimethylsilyl)-L-serinate as a thick, orange oil (0.615 g, 46%). ¹H NMR (400 MHz, CDCl₃): δ 8.22 (d, J=8.7 Hz, 1H), 8.05 (s, 1H), 7.94-7.85 (m, 2H), 7.62 (d, J=8.6 Hz, 2H), 7.50 (s, 1H), 4.85 (dt, J=8.7, 3.1 Hz, 1H), 4.22 (dd, J=10.1, 2.7 Hz, 1H), 3.95 (dd, J=10.1, 3.4 Hz, 1H), 3.79 (s, 3H), 3.30 (t, J=6.8 Hz, 2H), 2.41 (t, J=7.4 Hz, 2H), 1.84-1.72 (m, 2H), 1.72-1.59 (m, 2H), 1.54-1.43 (m, 2H), 0.92 (s, 9H), 0.08 (s, 3H), 0.06 (s, 3H).

### Methyl O-acetyl-N—(N-(2-(4-(6-azidohexanamido)phenyl)thiazole-4-carbonyl)-O-(tert-butyldimethylsilyl)-L-seryl)-L-serinate (25.5)

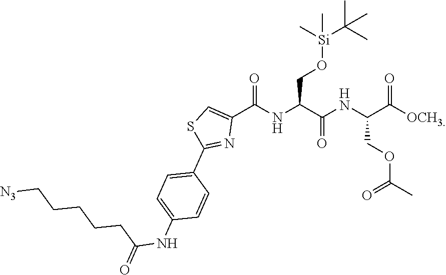 [SMILES: COC(=O)[C@H](COC(C)=O)NC(=O)[C@H](CO[Si](C)(C)C(C)(C)C)NC(=O)c1csc(-c2ccc(NC(=O)CCCCCN=[N+]=[N-])cc2)n1]

Using the procedure described in Example 15.6, methyl N-(2-(4-(6-azidohexanamido)phenyl)thiazole-4-carbonyl)-O-(tert-butyldimethylsilyl)-L-serinate (0.615 g, 1.07 mmole) was converted to methyl O-acetyl-N—(N-(2-(4-(6-azidohexanamido)phenyl)thiazole-4-carbonyl)-O-(tert-butyldimethylsilyl)-L-seryl)-L-serinate as a pale yellow oil (0.517 g, 69%). ¹H NMR (400 MHz, CDCl₃): δ 8.25 (dd, J=7.3, 4.4 Hz, 1H), 8.07 (d, J=3.4 Hz, 1H), 7.96-7.88 (m, 2H), 7.63 (d, J=8.3 Hz, 2H), 7.47 (d, J=7.7 Hz, 1H), 7.42 (d, J=7.6 Hz, 1H), 4.95-4.85 (m, 1H), 4.67 (td, J=7.2, 3.7 Hz, 1H), 4.55-4.45 (m, 1H), 4.34 (dd, J=11.4, 3.6 Hz, 1H), 4.22 (dt, J=9.9, 3.8 Hz, 1H), 3.81 (dd, J=9.8, 7.4 Hz, 1H), 3.77 (s, 3H), 3.31 (t, J=6.8 Hz, 2H), 2.42 (t, J=7.4 Hz, 2H), 2.01 (s, 3H), 1.85-1.74 (m, 2H), 1.71-1.64 (m, 2H), 1.56-1.43 (m, 2H), 0.96 (s, 9H), 0.16 (s, 3H), 0.15 (s, 3H).

### Methyl 2-(2-(2-(4-(6-azidohexanamido)phenyl)thiazole-4-carboxamido)acrylamido)acrylate (25)

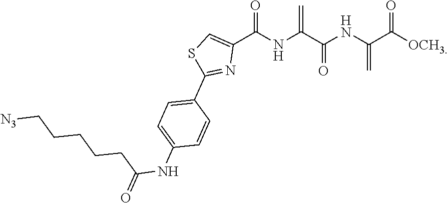 [SMILES: C=C(NC(=O)c1csc(-c2ccc(NC(=O)CCCCCN=[N+]=[N-])cc2)n1)C(=O)NC(=C)C(=O)OC]

Using the procedure as described for Compound 15, methyl O-acetyl-N—(N-(2-(4-(6-azidohexanamido)phenyl)thiazole-4-carbonyl)-O-(tert-butyldimethylsilyl)-L-seryl)-L-serinate (0.517 g, 0.734 mmole) converted to methyl 2-(2-(2-(4-(6-azidohexanamido)phenyl)thiazole-4-carboxamido)acrylamido)acrylate as a white solid (0.093 g, 25%). ¹H NMR (400 MHz, CDCl₃): δ 10.00 (s, 1H), 8.55 (s, 1H), 8.12 (s, 1H), 8.03-7.92 (m, 2H), 7.64 (d, J=8.3 Hz, 2H), 7.29 (s, 1H), 6.78 (d, J=2.2 Hz, 1H), 6.71 (s, 1H), 6.04 (d, J=1.3 Hz, 1H), 5.50 (t, J=1.9 Hz, 1H), 3.91 (s, 3H), 3.31 (t, J=6.8 Hz, 2H), 2.42 (t, J=7.4 Hz, 2H), 1.80 (tt, J=9.5, 6.5 Hz, 2H), 1.73-1.60 (m, 2H), 1.54-1.43 (m, 2H).

### Compound 26: Methyl 2-(2-(2-(4-((((2-methoxyethoxy)carbonyl)amino)methyl)phenyl)thiazole-4-carboxamido)acrylamido)acrylate

### 2-Methoxyethyl 1H-imidazole-1-carboxylate (26.1)

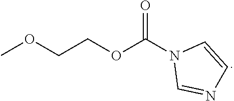

Following a literature procedure (RSC Advances, 2014, 4(25), 13012-13017) (27), 2-methoxyethanol (1.05 mL, 13.3 mmole) was dissolved in THE (26 mL). N,N′-Carbonyldiimidazole (2.560 g, 15.8 mmole) was added and the solution was stirred at RT for 20 hours. Water (100 mL) was added and the mixture was extracted with ethyl acetate (3×25 mL). The combined organics were dried over magnesium sulfate, filtered, and concentrated to provide 2-methoxyethyl 1H-imidazole-1-carboxylate as a colorless oil (2.757 g). ¹H NMR (400 MHz, CDCl₃): δ 8.20-8.12 (m, 1H), 7.45 (t, J=1.5 Hz, 1H), 7.07 (dd, J=1.7, 0.9 Hz, 1H), 4.60-4.52 (m, 2H), 3.76-3.69 (m, 2H), 3.42 (s, 3H).

### 2-Methoxyethyl (4-cyanobenzyl)carbamate (26.2)

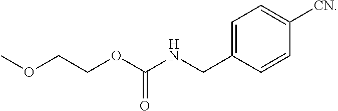 [SMILES: COCCOC(=O)NCc1ccc(C#N)cc1]

2-Methoxyethyl 1H-imidazole-1-carboxylate (1.261 g, 7.41 mmole) was dissolved in DMF (14 mL). 4-(Aminomethyl)benzonitrile hydrochloride (1.027 g, 7.77 mmole) and N,N-diisopropylethylamine (1.90 mL, 10.9 mmole) were added and the solution was heated to 70° C. for 2 ½ days, then was cooled to RT. Ethyl acetate (40 mL) was added and the mixture was washed with water (3×25 mL). The organics were dried over magnesium sulfate, filtered, and concentrated. The crude residue was purified by silica gel chromatography (40-80% ethyl acetate/hexane gradient) to provide 2-methoxyethyl (4-cyanobenzyl)carbamate as a yellow solid (0.888 g, 51%). ¹H NMR (400 MHz, CDCl₃): δ 7.67-7.58 (m, 2H), 7.40 (dq, J=7.4, 0.8 Hz, 2H), 5.23 (s, 1H), 4.43 (d, J=6.3 Hz, 2H), 4.31-4.24 (m, 2H), 3.64-3.53 (m, 2H), 3.40 (s, 3H).

### 2-Methoxyethyl (4-carbamothioylbenzyl)carbamate (26.3)

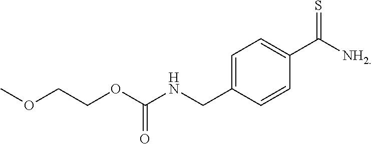 [SMILES: COCCOC(=O)NCc1ccc(C(N)=S)cc1]

2-Methoxyethyl (4-cyanobenzyl)carbamate (0.888 g, 3.79 mmole) was dissolved in pyridine (4 mL). Triethylamine (0.590 mL, 4.21 mmole) and ammonium sulfide (40% aqueous solution, 0.780 mL, 4.57 mmole) were added and the mixture was warmed to 50° C. for 5 hours, then was cooled to RT and concentrated. The residue was suspended in ethyl acetate (25 mL). The mixture was washed sequentially with 1N aq. HCl (2×15 mL) and water (1×15 mL). The aqueous layers were extracted with ethyl acetate (1×10 mL). The combined organics were dried over magnesium sulfate, filtered, and concentrated to provide 2-methoxyethyl (4-carbamothioylbenzyl)carbamate as a yellow solid (0.932 g, 92%). ¹H NMR (400 MHz, DMSO-d₆): δ9.81 (s, 1H), 9.44 (s, 1H), 7.87-7.82 (m, 2H), 7.82-7.75 (m, 1H), 7.27 (d, J=8.3 Hz, 2H), 4.20 (d, J=6.2 Hz, 2H), 4.12-4.04 (m, 2H), 3.53-3.44 (m, 2H), 3.25 (s, 3H).

### Ethyl 2-(4-((((2-methoxyethoxy)carbonyl)amino)methyl)phenyl)thiazole-4-carboxylate (26.4)

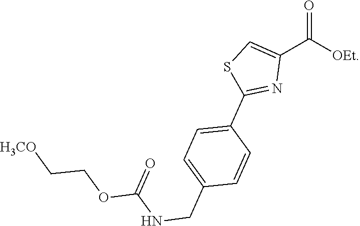 [SMILES: CCOC(=O)c1csc(-c2ccc(CNC(=O)OCCOC)cc2)n1]

2-Methoxyethyl (4-carbamothioylbenzyl)carbamate (0.932 g, 3.47 mmole) was dissolved in ethanol (7 mL). Ethyl bromopyruvate (0.480 mL, 3.83 mmole) was added and the mixture was heated to 80° C. for 2 ½ hours. The mixture was cooled to RT and concentrated. The residue was suspended in ethyl acetate (25 mL) and washed with water (1×25 mL), sat. aq. sodium bicarbonate (1×25 mL), water (1×25 mL), and sat. aq. sodium chloride (1×25 mL). The organics were dried over magnesium sulfate, filtered, and concentrated. The crude residue was purified by silica gel chromatography (40-80% ethyl acetate/hexane gradient) to provide ethyl 2-(4-((((2-methoxyethoxy)carbonyl)amino)methyl)phenyl)thiazole-4-carboxylate as a white solid (0.887 g, 70%). ¹H NMR (400 MHz, CDCl₃): δ 8.15 (s, 1H), 8.03-7.92 (m, 2H), 7.39-7.33 (m, 2H), 5.16 (s, 1H), 4.49-4.39 (m, 4H), 4.32-4.23 (m, 2H), 3.64-3.56 (m, 2H), 3.40 (s, 3H), 1.43 (t, J=7.1 Hz, 3H).

### Methyl O-(tert-butyldimethylsilyl)-N-(2-(4-((((2-methoxyethoxy)carbonyl)amino)methyl)phenyl)thiazole-4-carbonyl)-L-serinate (26.5)

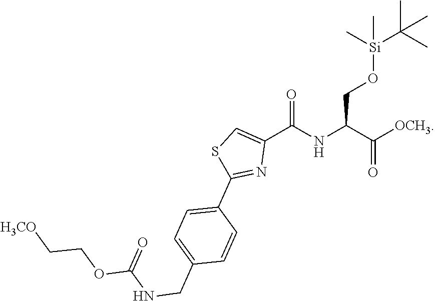 [SMILES: COCCOC(=O)NCc1ccc(-c2nc(C(=O)N[C@@H](CO[Si](C)(C)C(C)(C)C)C(=O)OC)cs2)cc1]

Using the procedure described in Example 19.3, ethyl 2-(4-((((2-methoxyethoxy)carbonyl)amino)methyl)phenyl)thiazole-4-carboxylate (0.887 g, 2.43 mmole) was converted to methyl O-(tert-butyldimethylsilyl)-N-(2-(4-((((2-methoxyethoxy)carbonyl)amino)methyl)phenyl)thiazole-4-carbonyl)-L-serinate as a thick, colorless oil (1.202 g, 90%). ¹H NMR (400 MHz, CDCl₃): δ 8.22 (d, J=8.7 Hz, 1H), 8.09 (s, 1H), 7.97-7.88 (m, 2H), 7.40-7.34 (m, 2H), 5.21 (s, 1H), 4.90-4.82 (m, 1H), 4.43 (dd, J=6.3, 2.1 Hz, 2H), 4.33-4.25 (m, 2H), 4.21 (dd, J=10.1, 2.6 Hz, 1H), 3.95 (dd, J=10.1, 3.4 Hz, 1H), 3.79 (s, 3H), 3.65-3.56 (m, 2H), 3.40 (s, 3H), 0.92 (s, 9H), 0.08 (s, 3H), 0.06 (s, 3H).

### Methyl O-acetyl-N—(O-(tert-butyldimethylsilyl)-N-(2-(4-((((2-methoxyethoxy)carbonyl)amino)methyl)phenyl)thiazole-4-carbonyl)-L-seryl)-L-serinate (26.6)

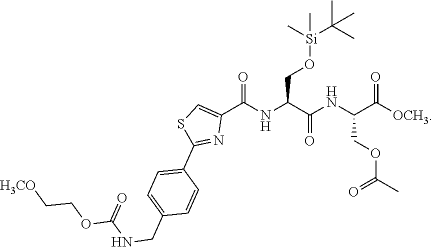 [SMILES: COCCOC(=O)NCc1ccc(-c2nc(C(=O)N[C@@H](CO[Si](C)(C)C(C)(C)C)C(=O)N[C@@H](COC(C)=O)C(=O)OC)cs2)cc1]

Using the procedure described in Example 15.6, methyl O-(tert-butyldimethylsilyl)-N-(2-(4-((((2-methoxyethoxy)carbonyl)amino)methyl)phenyl)thiazole-4-carbonyl)-L-serinate (1.202 g, 2.18 mmole) was converted to methyl O-acetyl-N—(O-(tert-butyldimethylsilyl)-N-(2-(4-((((2-methoxyethoxy)carbonyl)amino)methyl)phenyl)thiazole-4-carbonyl)-L-seryl)-L-serinate as a colorless oil (1.001 g, 67%). ¹H NMR (400 MHz, CDCl₃): δ 8.26 (d, J=7.1 Hz, 1H), 8.11 (d, J=2.6 Hz, 1H), 7.94 (dd, J=8.3, 1.8 Hz, 2H), 7.46 (d, J=7.8 Hz, 1H), 7.41-7.32 (m, 2H), 5.17 (s, 1H), 4.90 (dq, J=7.0, 3.5 Hz, 1H), 4.71-4.62 (m, 1H), 4.49 (ddd, J=11.2, 7.2, 3.9 Hz, 1H), 4.43 (d, J=6.2 Hz, 2H), 4.33 (dd, J=11.4, 3.6 Hz, 1H), 4.31-4.26 (m, 2H), 4.23 (dt, J=9.9, 3.6 Hz, 1H), 3.84-3.79 (m, 1H), 3.77 (d, J=4.0 Hz, 3H), 3.66-3.57 (m, 2H), 3.41 (s, 3H), 2.01 (s, 3H), 0.96 (s, 9H), 0.16 (s, 3H), 0.16 (s, 3H).

### Methyl 2-(2-(2-(4-((((2-methoxyethoxy)carbonyl)amino)methyl)phenyl)thiazole-4-carboxamido)acrylamido)acrylate (26)

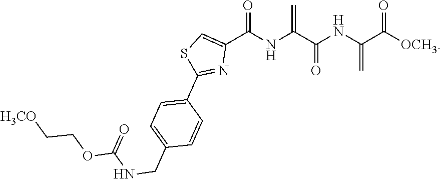 [SMILES: C=C(NC(=O)c1csc(-c2ccc(CNC(=O)OCCOC)cc2)n1)C(=O)NC(=C)C(=O)OC]

Using the procedure described for Compound 15, methyl O-acetyl-N—(O-(tert-butyldimethylsilyl)-N-(2-(4-((((2-methoxyethoxy)carbonyl)amino)methyl)phenyl)thiazole-4-carbonyl)-L-seryl)-L-serinate (1.001 g, 1.47 mmole) was converted to methyl 2-(2-(2-(4-((((2-methoxyethoxy)carbonyl)amino)methyl)phenyl)thiazole-4-carboxamido)acrylamido)acrylate as a white solid (0.103 g, 14%). ¹H NMR (400 MHz, CDCl₃): δ 10.01 (s, 1H), 8.55 (s, 1H), 8.14 (s, 1H), 8.04-7.92 (m, 2H), 7.44-7.33 (m, 2H), 6.78 (d, J=2.2 Hz, 1H), 6.71 (s, 1H), 6.03 (d, J=1.3 Hz, 1H), 5.50 (t, J=1.9 Hz, 1H), 5.18 (s, 1H), 4.43 (d, J=6.1 Hz, 2H), 4.34-4.24 (m, 2H), 3.90 (s, 3H), 3.67-3.56 (m, 2H), 3.41 (s, 3H).

### Compound 27: Methyl 2-(2-(2-(3-((2-methoxyethoxy)methyl)phenyl)thiazole-4-carboxamido)acrylamido)acrylate

### 3-((2-Methoxyethoxy)methyl)benzonitrile (27.1)

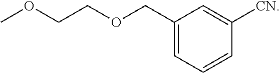

2-Methoxyethanol (1.05 mL, 13.3 mmole) was dissolved in DMF (20 mL) and cooled to 0° C. Sodium hydride (60% dispersion in mineral oil, 0.532 g, 13.3 mmole) was added portionwise (gas evolution was observed) and the mixture was stirred at 0° for 60 minutes. 3-(Bromomethyl)benzonitrile (2.046 g, 10.4 mmole) was added and the mixture was warmed to RT and stirred for 18 hours. Ethyl acetate (50 mL) was added and the mixture was washed with water (3×25 mL). The combined organics were dried over magnesium sulfate, filtered, and concentrated. The crude residue was purified by silica gel chromatography (10-40% ethyl acetate/hexane gradient) to provide 3-((2-methoxyethoxy)methyl)benzonitrile as a colorless oil (0.844, 42%). ¹H NMR (400 MHz, CDCl₃): δ 7.67 (td, J=1.7, 0.8 Hz, 1H), 7.62-7.54 (m, 2H), 7.45 (t, J=7.7 Hz, 1H), 4.64-4.56 (m, 2H), 3.71-3.63 (m, 2H), 3.63-3.56 (m, 2H), 3.41 (s, 3H).

### 3-((2-Methoxyethoxy)methyl)benzothioamide (27.2)

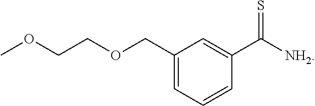

3-((2-Methoxyethoxy)methyl)benzonitrile (0.844 g, 4.41 mmole) was dissolved in pyridine (5 mL). Triethylamine (0.740 mL, 5.28 mmole) and ammonium sulfide (40% aqueous solution, 1.05 mL, 6.15 mmole) were added and the mixture was heated to 50° C. for 5 hours and was cooled to RT. The mixture was concentrated and the residue was suspended in ethyl acetate (25 mL). The mixture was washed with 1N aq. HCl (2×25 mL), then dried over magnesium sulfate, filtered, and concentrated to provide 3-((2-methoxyethoxy)methyl)benzothioamide as a yellow oil (0.797 g, 80%). ¹H NMR (400 MHz, DMSO-d₆): δ 9.86 (s, 1H), 9.50 (s, 1H), 7.85 (q, J=1.4, 0.9 Hz, 1H), 7.80-7.72 (m, 1H), 7.45 (dt, J=7.6, 1.5 Hz, 1H), 7.39 (t, J=7.6 Hz, 1H), 4.52 (s, 2H), 3.62-3.54 (m, 2H), 3.53-3.45 (m, 2H), 3.26 (s, 3H).

### Ethyl 2-(3-((2-methoxyethoxy)methyl)phenyl)thiazole-4-carboxylate (27.3)

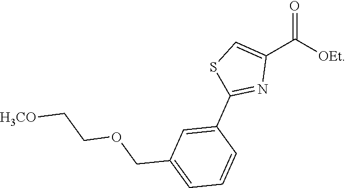 [SMILES: CCOC(=O)c1csc(-c2cccc(COCCOC)c2)n1]

3-((2-Methoxyethoxy)methyl)benzothioamide (0.797 g, 3.54 mmole) was dissolved in ethanol. Ethyl bromopyruvate (0.490 mL, 3.90 mmole) was added and the mixture was heated to 80° C. for 4 hours. The mixture was cooled to RT and concentrated. The crude residue was purified by silica gel chromatography (20-50% ethyl acetate/hexane gradient) to provide ethyl 2-(3-((2-methoxyethoxy)methyl)phenyl)thiazole-4-carboxylate as a yellow oil (0.421 g, 37%). ¹H NMR (400 MHz, CDCl₃): δ 8.16 (s, 1H), 8.00 (td, J=1.7, 0.7 Hz, 1H), 7.92 (dt, J=7.4, 1.7 Hz, 1H), 7.51-7.39 (m, 2H), 4.64 (s, 2H), 4.45 (q, J=7.1 Hz, 2H), 3.70-3.62 (m, 2H), 3.62-3.55 (m, 2H), 3.41 (s, 3H), 1.44 (t, J=7.1 Hz, 3H).

### Methyl O-(tert-butyldimethylsilyl)-N-(2-(3-((2-methoxyethoxy)methyl)phenyl)thiazole-4-carbonyl)-L-serinate (27.4)

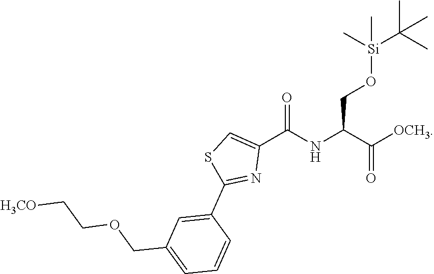 [SMILES: COCCOCc1cccc(-c2nc(C(=O)N[C@@H](CO[Si](C)(C)C(C)(C)C)C(=O)OC)cs2)c1]

Using the procedure described in Example 19.3, ethyl 2-(3-((2-methoxyethoxy)methyl)phenyl)thiazole-4-carboxylate (0.421 g, 1.31 mmole) was converted to methyl O-(tert-butyldimethylsilyl)-N-(2-(3-((2-methoxyethoxy)methyl)phenyl)thiazole-4-carbonyl)-L-serinate as a thick, colorless oil (0.475 g, 71%). ¹H NMR (400 MHz, CDCl₃): δ 8.21 (d, J=8.7 Hz, 1H), 8.11 (s, 1H), 7.95-7.87 (m, 2H), 7.52-7.40 (m, 2H), 4.87 (ddd, J=8.7, 3.4, 2.7 Hz, 1H), 4.65 (s, 2H), 4.22 (dd, J=10.1, 2.7 Hz, 1H), 3.95 (dd, J=10.1, 3.4 Hz, 1H), 3.79 (s, 3H), 3.71-3.64 (m, 2H), 3.64-3.58 (m, 2H), 3.41 (s, 3H), 0.92 (s, 9H), 0.08 (s, 3H), 0.06 (s, 3H).

### Methyl O-acetyl-N—(O-(tert-butyldimethylsilyl)-N-(2-(3-((2-methoxyethoxy)methyl)phenyl)thiazole-4-carbonyl)-L-seryl)-L-serinate (27.5)

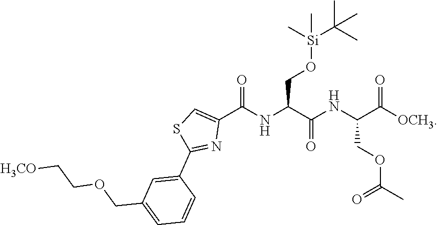 [SMILES: COCCOCc1cccc(-c2nc(C(=O)N[C@@H](CO[Si](C)(C)C(C)(C)C)C(=O)N[C@@H](COC(C)=O)C(=O)OC)cs2)c1]

Using the procedure described in Example 15.6, methyl O-(tert-butyldimethylsilyl)-N-(2-(3-((2-methoxyethoxy)methyl)phenyl)thiazole-4-carbonyl)-L-serinate (0.475 g, 0.934 mmole) was converted to methyl O-acetyl-N—(O-(tert-butyldimethylsilyl)-N-(2-(3-((2-methoxyethoxy)methyl)phenyl)thiazole-4-carbonyl)-L-seryl)-L-serinate as a colorless oil (0.415 g, 70%). ¹H NMR (400 MHz, CDCl₃): δ 8.24 (d, J=7.1 Hz, 1H), 8.12 (d, J=2.5 Hz, 1H), 7.92 (ddt, J=5.2, 3.4, 1.7 Hz, 2H), 7.51-7.40 (m, 3H), 4.95-4.85 (m, 1H), 4.72-4.66 (m, 1H), 4.65 (s, 2H), 4.49 (ddd, J=11.3, 6.1, 3.9 Hz, 1H), 4.34 (dd, J=11.4, 3.6 Hz, 1H), 4.22 (ddd, J=9.8, 3.8, 2.8 Hz, 1H), 3.84-3.79 (m, 1H), 3.77 (s, 3H), 3.70-3.64 (m, 2H), 3.64-3.56 (m, 2H), 3.41 (s, 3H), 2.02 (s, 3H), 0.96 (s, 9H), 0.17 (s, 3H), 0.16 (s, 3H).

### Methyl 2-(2-(2-(3-((2-methoxyethoxy)methyl)phenyl)thiazole-4-carboxamido)acrylamido)acrylate (27)

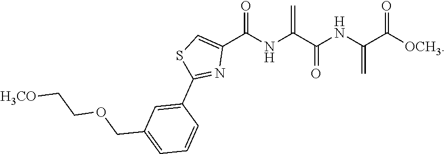 [SMILES: C=C(NC(=O)c1csc(-c2cccc(COCCOC)c2)n1)C(=O)NC(=C)C(=O)OC]

Using the procedure as described for Compound 15, methyl O-acetyl-N—(O-(tert-butyldimethylsilyl)-N-(2-(3-((2-methoxyethoxy)methyl)phenyl)thiazole-4-carbonyl)-L-seryl)-L-serinate (0.415 g, 0.651 mmole) was converted to methyl 2-(2-(2-(3-((2-methoxyethoxy)methyl)phenyl)thiazole-4-carboxamido)acrylamido)acrylate as a white solid (0.153 g, 53%). ¹H NMR (400 MHz, CDCl₃): δ 10.00 (s, 1H), 8.55 (s, 1H), 8.16 (s, 1H), 7.97 (dt, J=6.5, 2.3 Hz, 1H), 7.94 (d, J=1.7 Hz, 1H), 7.51-7.42 (m, 2H), 6.78 (d, J=2.2 Hz, 1H), 6.72 (s, 1H), 6.03 (d, J=1.3 Hz, 1H), 5.51 (t, J=1.9 Hz, 1H), 4.67 (s, 2H), 3.90 (s, 3H), 3.74-3.66 (m, 2H), 3.66-3.57 (m, 2H), 3.42 (s, 3H).

### Compound 28: Methyl 2-(2-(2-(3-(isobutylcarbamoyl)piperidin-1-yl)thiazole-4-carboxamido)acrylamido)acrylate

### Tert-butyl 3-(isobutylcarbamoyl)piperidine-1-carboxylate (28.1)

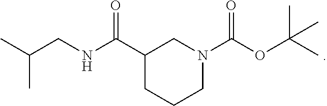 [SMILES: CC(C)CNC(=O)C1CCCN(C(=O)OC(C)(C)C)C1]

1-(Tert-butoxycarbonyl)piperidine-3-carboxylic acid (0.754 g, 3.29 mmole) was dissolved in DCM (6.5 mL). Isobutylamine (0.390 mL, 3.92 mmole) and N,N-diisopropylethylamine (1.15 mL, 6.60 mmole) were added followed by HOBt·H₂O (0.603 g, 3.94 mmole) and EDC·HCl (0.750 g, 3.91 mmole). The mixture was stirred at RT for 3 days. Water (25 mL) was added and the mixture was extracted with DCM (3×10 mL). The combined organics were dried over magnesium sulfate, filtered, and concentrated. The crude residue was purified by silica gel chromatography (20-50% ethyl acetate/hexane gradient) to provide tert-butyl 3-(isobutylcarbamoyl)piperidine-1-carboxylate as a white solid (0.817 g, 87%). ¹H NMR (400 MHz, CDCl₃): δ 4.07-3.61 (m, 2H), 3.26 (s, 1H), 3.08 (t, J=6.4 Hz, 2H), 2.29 (s, 1H), 1.85 (d, J=29.1 Hz, 2H), 1.76 (p, J=6.7 Hz, 1H), 1.62 (s, 1H), 1.46 (s, 9H), 0.91 (dd, J=6.7, 1.1 Hz, 6H).

### Ethyl 2-(3-(isobutylcarbamoyl)piperidin-1-yl)thiazole-4-carboxylate (28.2)

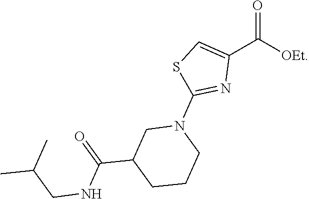 [SMILES: CCOC(=O)c1csc(N2CCCC(C(=O)NCC(C)C)C2)n1]

Tert-butyl 3-(isobutylcarbamoyl)piperidine-1-carboxylate (0.817 g, 2.87 mmole) was dissolved in DCM (5.5 mL). HCl (4M solution in 1,4-dioxane, 2.80 mL, 11.2 mmole) was added and the mixture was stirred at RT for 3 hours, then was concentrated. The residue was dissolved in DMA (3 mL) and ethyl 2-bromothiazole-4-carboxylate (0.749 g, 3.17 mmole) and triethylamine (0.490 mL, 3.50 mmole) were added. The mixture was heated to 80° C. for 18 hours and was cooled to RT. Ethyl acetate (25 mL) was added and the mixture was washed with water (3×25 mL). The organics were dried over magnesium sulfate, filtered, and concentrated. The crude residue was purified by silica gel chromatography (30-70% ethyl acetate/hexane gradient) to provide ethyl 2-(3-(isobutylcarbamoyl)piperidin-1-yl)thiazole-4-carboxylate as a white solid (0.445 g, 46%). ¹H NMR (400 MHz, CDCl₃): δ 7.43 (s, 1H), 5.93 (s, 1H), 4.35 (q, J=7.1 Hz, 2H), 4.05 (ddd, J=13.3, 3.2, 2.0 Hz, 1H), 3.83-3.74 (m, 1H), 3.47 (dd, J=13.2, 9.5 Hz, 1H), 3.24-3.14 (m, 1H), 3.14-3.01 (m, 2H), 2.45 (tt, J=9.3, 4.5 Hz, 1H), 1.93 (ddd, J=9.5, 8.0, 4.3 Hz, 2H), 1.85-1.71 (m, 2H), 1.68-1.59 (m, 1H), 1.37 (t, J=7.1 Hz, 3H), 0.89 (d, J=6.7 Hz, 6H).

### Methyl O-(tert-butyldimethylsilyl)-N-(2-(3-(isobutylcarbamoyl)piperidin-1-yl)thiazole-4-carbonyl)-L-serinate (28.3)

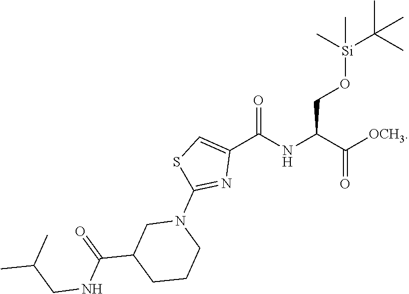 [SMILES: COC(=O)[C@H](CO[Si](C)(C)C(C)(C)C)NC(=O)c1csc(N2CCCC(C(=O)NCC(C)C)C2)n1]

Using the procedure described in Example 19.3, ethyl 2-(3-(isobutylcarbamoyl)piperidin-1-yl)thiazole-4-carboxylate (0.445 g, 1.34 mmole) was converted to a diastereomeric mixture of methyl O-(tert-butyldimethylsilyl)-N-(2-(3-(isobutylcarbamoyl)piperidin-1-yl)thiazole-4-carbonyl)-L-serinate as a thick, colorless oil (0.470 g, 67%). ¹H NMR (400 MHz, CDCl₃): δ 7.93 (dd, J=17.6, 8.7 Hz, 1H), 7.38 (d, J=2.1 Hz, 1H), 5.84 (dd, J=13.7, 6.7 Hz, 1H), 4.79 (ddt, J=9.5, 6.4, 3.0 Hz, 1H), 4.19-4.13 (m, 1H), 3.99 (dd, J=13.2, 4.0 Hz, 1H), 3.93-3.79 (m, 2H), 3.76 (d, J=0.8 Hz, 3H), 3.47 (ddd, J=48.4, 13.3, 9.5 Hz, 1H), 3.25-3.00 (m, 3H), 2.46 (qt, J=8.7, 4.1 Hz, 1H), 1.93 (dq, J=11.6, 3.6, 2.3 Hz, 2H), 1.77 (dtd, J=13.4, 6.8, 5.1 Hz, 2H), 1.67 (dt, J=9.2, 4.6 Hz, 2H), 0.90 (dd, J=6.7, 4.7 Hz, 6H), 0.87 (d, J=1.6 Hz, 9H), 0.04 (d, 3H), 0.02 (d, 3H).

### Methyl O-acetyl-N—(O-(tert-butyldimethylsilyl)-N-(2-(3-(isobutylcarbamoyl)piperidin-1-yl)thiazole-4-carbonyl)-L-seryl)-L-serinate (28.4)

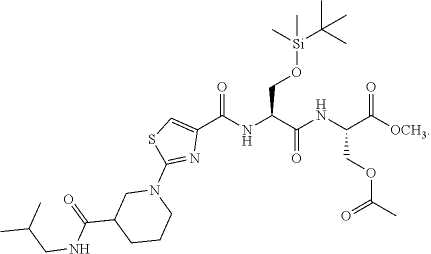 [SMILES: COC(=O)[C@H](COC(C)=O)NC(=O)[C@H](CO[Si](C)(C)C(C)(C)C)NC(=O)c1csc(N2CCCC(C(=O)NCC(C)C)C2)n1]

Using the procedure described in Example 15.6, methyl O-(tert-butyldimethylsilyl)-N-(2-(3-(isobutylcarbamoyl)piperidin-1-yl)thiazole-4-carbonyl)-L-serinate (0.470 g, 0.892 mmole) was converted to methyl O-acetyl-N—(O-(tert-butyldimethylsilyl)-N-(2-(3-(isobutylcarbamoyl)piperidin-1-yl)thiazole-4-carbonyl)-L-seryl)-L-serinate as a colorless oil (0.422 g, 72%). ¹H NMR (400 MHz, CDCl₃): δ 7.98 (dd, J=16.3, 6.9 Hz, 1H), 7.46 (dd, J=14.0, 7.9 Hz, 1H), 7.40 (d, J=1.3 Hz, 1H), 6.03-5.72 (m, 1H), 4.86 (ddt, J=7.6, 5.4, 3.8 Hz, 1H), 4.62-4.53 (m, 1H), 4.47 (ddd, J=11.3, 8.4, 3.9 Hz, 1H), 4.37-4.25 (m, 1H), 4.22-4.15 (m, 1H), 3.88 (dd, J=13.1, 4.5 Hz, 1H), 3.81-3.68 (m, 4H), 3.50-3.32 (m, 1H), 3.25-2.99 (m, 3H), 2.47 (ddt, J=15.5, 5.8, 3.4 Hz, 1H), 2.04-1.99 (m, 3H), 1.98-1.87 (m, 2H), 1.79 (ttt, J=13.5, 6.7, 3.1 Hz, 2H), 0.92 (d, J=1.6 Hz, 8H), 0.90 (dd, J=6.6, 0.8 Hz, 6H), 0.14 (dt, J=6.1, 3.3 Hz, 6H).

### Methyl 2-(2-(2-(3-(isobutylcarbamoyl)piperidin-1-yl)thiazole-4-carboxamido)acrylamido)acrylate (28)

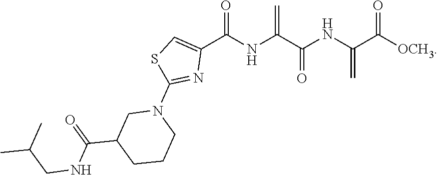 [SMILES: C=C(NC(=O)c1csc(N2CCCC(C(=O)NCC(C)C)C2)n1)C(=O)NC(=C)C(=O)OC]

Using the procedure as described for Compound 15, methyl O-acetyl-N—(O-(tert-butyldimethylsilyl)-N-(2-(3-(isobutylcarbamoyl)piperidin-1-yl)thiazole-4-carbonyl)-L-seryl)-L-serinate (0.422 g, 0.643 mmole) was converted to methyl 2-(2-(2-(3-(isobutylcarbamoyl)piperidin-1-yl)thiazole-4-carboxamido)acrylamido)acrylate as a white solid (0.139 g, 47%). ¹H NMR (400 MHz, CDCl₃): δ 9.78 (s, 1H), 8.52 (s, 1H), 7.44 (s, 1H), 6.71 (d, J=2.1 Hz, 1H), 6.63 (s, 1H), 6.00 (d, J=1.3 Hz, 1H), 5.92 (t, J=6.0 Hz, 1H), 5.43 (t, J=1.9 Hz, 1H), 4.07 (dd, J=13.3, 4.0 Hz, 1H), 3.90 (s, 3H), 3.77 (ddd, J=13.7, 5.1, 3.8 Hz, 1H), 3.47-3.37 (m, 1H), 3.23 (ddd, J=13.2, 11.0, 3.4 Hz, 1H), 3.19-3.04 (m, 2H), 2.60-2.47 (m, 1H), 1.96 (td, J=8.3, 7.7, 3.9 Hz, 2H), 1.87-1.73 (m, 2H), 1.73-1.62 (m, 1H), 0.90 (d, J=6.7 Hz, 6H).

### Compound 29: Methyl 2-(2-(2-(4-((3-isopropoxypropyl)carbamoyl)piperidin-1-yl)thiazole-4-carboxamido)acrylamido)acrylate

### Ethyl 2-(4-(tert-butoxycarbonyl)piperidin-1-yl)thiazole-4-carboxylate (29.1)

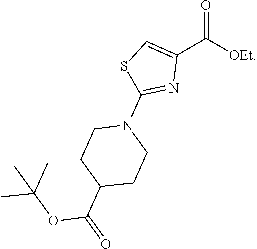 [SMILES: CCOC(=O)c1csc(N2CCC(C(=O)OC(C)(C)C)CC2)n1]

Tert-butyl piperidine-4-carboxylate hydrochloride (2.000 g, 9.02 mmole) was dissolved in DMA (10 mL). Ethyl 2-bromothiazole-4-carboxylate (2.251 g, 9.53 mmole) and triethylamine (1.40 mL, 9.99 mmole) were added and the mixture was heated to 80° C. for 18 hours, then was cooled to RT. Ethyl acetate (50 mL) was added and the mixture was washed with water (3×25 mL). The organics were dried over magnesium sulfate, filtered, and concentrated. The crude residue was purified by silica gel chromatography (10-30% ethyl acetate/hexane gradient) to provide ethyl 2-(4-(tert-butoxycarbonyl)piperidin-1-yl)thiazole-4-carboxylate as a pale yellow solid (1.735 g, 57%). ¹H NMR (400 MHz, CDCl₃): δ 7.43 (s, 1H), 4.35 (q, J=7.1 Hz, 2H), 4.02-3.91 (m, 2H), 3.14 (ddd, J=13.0, 11.1, 3.2 Hz, 2H), 2.43 (tt, J=10.7, 3.8 Hz, 1H), 1.98 (dtt, J =13.5, 3.9, 2.0 Hz, 2H), 1.85-1.73 (m, 2H), 1.45 (s, 9H), 1.37 (t, J=7.1 Hz, 3H).

### Ethyl 2-(4-((3-isopropoxypropyl)carbamoyl)piperidin-1-yl)thiazole-4-carboxylate (29.2)

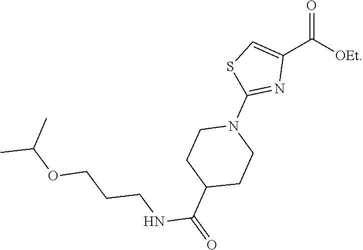 [SMILES: CCOC(=O)c1csc(N2CCC(C(=O)NCCCOC(C)C)CC2)n1]

Ethyl 2-(4-(tert-butoxycarbonyl)piperidin-1-yl)thiazole-4-carboxylate (0.557 g, 1.64 mmole) was dissolved in DCM (3.2 mL). HCl (4M solution in 1,4-dioxane, 1.60 mL, 6.40 mmole) was added and the solution was stirred at RT for 18 hours, then was concentrated. The residue was suspended in DCM (3.2 mL). N,N-Diisopropylethylamine (0.560 mL, 3.22 mmole) was added followed by 3-isopropoxypropan-1-amine (0.270 mL, 1.95 mmole), HOBt·H₂O (0.295 g, 1.93 mmole), and EDC·HCl (0.382 g, 1.99 mmole). The mixture was stirred at RT for 18 hours and water (25 mL) was added. The mixture was extracted with DCM (3×10 mL). The combined organics were dried over magnesium sulfate, filtered, and concentrated. The crude residue was purified by silica gel chromatography (70-100% ethyl acetate/hexane gradient) to provide ethyl 2-(4-((3-isopropoxypropyl)carbamoyl)piperidin-1-yl)thiazole-4-carboxylate as a white solid (0.497 g, 79%). ¹H NMR (400 MHz, CDCl₃): δ 7.44 (s, 1H), 6.46 (s, 1H), 4.35 (q, J =7.1 Hz, 2H), 4.08 (dt, J=13.4, 4.2 Hz, 2H), 3.62-3.50 (m, 3H), 3.44-3.34 (m, 2H), 3.07 (ddd, J=13.0, 11.8, 3.1 Hz, 2H), 2.27 (tt, J=11.5, 3.6 Hz, 1H), 2.02-1.92 (m, 2H), 1.86-1.71 (m, 4H), 1.37 (t, J=7.1 Hz, 3H), 1.16 (d, J=6.1 Hz, 6H).

### Methyl O-(tert-butyldimethylsilyl)-N-(2-(4-((3-isopropoxypropyl)carbamoyl)piperidin-1-yl)thiazole-4-carbonyl)-L-serinate (29.3)

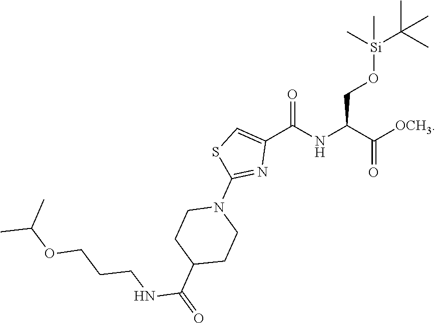 [SMILES: COC(=O)[C@H](CO[Si](C)(C)C(C)(C)C)NC(=O)c1csc(N2CCC(C(=O)NCCCOC(C)C)CC2)n1]

Using the procedure described in Example 19.3, ethyl 2-(4-((3-isopropoxypropyl)carbamoyl)piperidin-1-yl)thiazole-4-carboxylate (0.497 g, 1.30 mmole) was converted to methyl O-(tert-butyldimethylsilyl)-N-(2-(4-((3-isopropoxypropyl)carbamoyl)piperidin-1-yl)thiazole-4-carbonyl)-L-serinate as a thick, colorless oil (0.418 g, 56%). ¹H NMR (400 MHz, CDCl₃): δ 7.96 (d, J=8.8 Hz, 1H), 7.38 (s, 1H), 6.48 (t, J=5.2 Hz, 1H), 4.78 (ddd, J=8.8, 3.4, 2.6 Hz, 1H), 4.19-4.14 (m, 1H), 4.08-3.96 (m, 2H), 3.88 (dd, J=10.0, 3.4 Hz, 1H), 3.76 (s, 3H), 3.62-3.51 (m, 3H), 3.44-3.35 (m, 2H), 3.12-2.98 (m, 2H), 2.28 (tt, J=11.5, 3.6 Hz, 1H), 2.01-1.91 (m, 2H), 1.87-1.70 (m, 5H), 1.17 (d, J=6.1 Hz, 6H), 0.88 (s, 9H), 0.05 (s, 3H), 0.03 (s, 3H).

### Methyl O-acetyl-N—(O-(tert-butyldimethylsilyl)-N-(2-(4-((3-isopropoxypropyl)carbamoyl)piperidin-1-yl)thiazole-4-carbonyl)-L-seryl)-L-serinate (29.4)

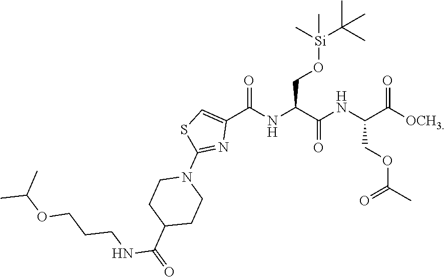 [SMILES: COC(=O)[C@H](COC(C)=O)NC(=O)[C@H](CO[Si](C)(C)C(C)(C)C)NC(=O)c1csc(N2CCC(C(=O)NCCCOC(C)C)CC2)n1]

Using the procedure described in Example 15.6, methyl O-(tert-butyldimethylsilyl)-N-(2-(4-((3-isopropoxypropyl)carbamoyl)piperidin-1-yl)thiazole-4-carbonyl)-L-serinate (0.418 g, 0.732 mmole) was converted to methyl O-acetyl-N—(O-(tert-butyldimethylsilyl)-N-(2-(4-((3-isopropoxypropyl)carbamoyl)piperidin-1-yl)thiazole-4-carbonyl)-L-seryl)-L-serinate as a colorless oil (0.276 g, 54%). ¹H NMR (400 MHz, CDCl₃): δ 7.99 (d, J=7.1 Hz, 1H), 7.43 (d, J=7.8 Hz, 1H), 7.39 (d, J=3.0 Hz, 1H), 6.47 (t, J=5.1 Hz, 1H), 4.87 (dq, J=8.2, 4.0 Hz, 1H), 4.63-4.54 (m, 1H), 4.51-4.43 (m, 1H), 4.31 (dd, J=11.4, 3.7 Hz, 1H), 4.23-4.16 (m, 1H), 4.03 (tt, J=15.8, 2.7 Hz, 2H), 3.76 (d, J=2.6 Hz, 3H), 3.75-3.69 (m, 1H), 3.62-3.52 (m, 3H), 3.40 (dt, J=6.0, 5.0 Hz, 2H), 3.05 (td, J=12.6, 3.0 Hz, 2H), 2.28 (tt, J=11.5, 3.6 Hz, 1H), 2.02 (d, J=2.8 Hz, 3H), 2.00-1.90 (m, 2H), 1.85-1.72 (m, 4H), 1.17 (d, J=6.1 Hz, 6H), 0.92 (s, 9H), 0.14 (s, 3H), 0.13 (s, 3H).

### Methyl 2-(2-(2-(4-((3-isopropoxypropyl)carbamoyl)piperidin-1-yl)thiazole-4-carboxamido)acrylamido)acrylate (29)

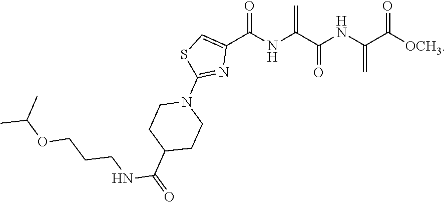 [SMILES: C=C(NC(=O)c1csc(N2CCC(C(=O)NCCCOC(C)C)CC2)n1)C(=O)NC(=C)C(=O)OC]

Using the procedure as described for Compound 15, methyl O-acetyl-N—(O-(tert-butyldimethylsilyl)-N-(2-(4-((3-isopropoxypropyl)carbamoyl)piperidin-1-yl)thiazole-4-carbonyl)-L-seryl)-L-serinate (0.276 g, 0.394 mmole) was converted to methyl 2-(2-(2-(4-((3-isopropoxypropyl)carbamoyl)piperidin-1-yl)thiazole-4-carboxamido)acrylamido)acrylate as a white solid (0.088 g, 44%). ¹H NMR (400 MHz, CDCl₃): δ 9.72 (s, 1H), 8.51 (s, 1H), 7.43 (s, 1H), 6.72 (d, J=2.1 Hz, 1H), 6.66 (s, 1H), 6.46 (s, 1H), 6.00 (d, J=1.3 Hz, 1H), 5.43 (t, J=1.8 Hz, 1H), 4.10-4.01 (m, 2H), 3.89 (s, 3H), 3.63-3.50 (m, 3H), 3.46-3.34 (m, 2H), 3.08 (ddd, J=13.0, 11.8, 3.0 Hz, 2H), 2.29 (tt, J=11.5, 3.7 Hz, 1H), 2.02-1.92 (m, 2H), 1.89-1.72 (m, 4H), 1.17 (d, J=6.1 Hz, 6H).

### Compound 30: Methyl 2-(2-(2-(4-(((benzylcarbamoyl)oxy)methyl)phenyl)thiazole-4-carboxamido)acrylamido)acrylate

### 4-Cyanobenzyl 1H-imidazole-1-carboxylate (30.1)

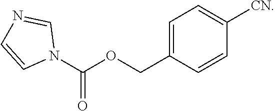 [SMILES: N#Cc1ccc(COC(=O)n2ccnc2)cc1]

Following a literature procedure (RSC Adv., 2014, 4, 13012-13017), 4-(hydroxymethyl)benzonitrile (2.000 g, 15.0 mmole) was dissolved in THF (30 mL). N,N′-Carbonyldiimidazole (2.924 g, 18.0 mmole) was added and the solution was stirred at RT for 2¹2 days. Water (50 mL) was added and the mixture was extracted with ethyl acetate (3×15 mL). The combined organics were washed sequentially with water (1×25 mL) and sat. aq. sodium chloride (1×25 mL), then dried over magnesium sulfate, filtered, and concentrated to provide 4-cyanobenzyl 1H-imidazole-1-carboxylate as a tan solid (3.489 g, 100%). ¹H NMR (400 MHz, CDCl₃): δ 8.16 (t, J=1.1 Hz, 1H), 7.77-7.70 (m, 2H), 7.60-7.52 (m, 2H), 7.44 (t, J=1.5 Hz, 1H), 7.13-7.05 (m, 1H), 5.48 (s, 2H).

### 4-Cyanobenzyl benzylcarbamate (30.2)

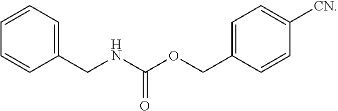 [SMILES: N#Cc1ccc(COC(=O)NCc2ccccc2)cc1]

4-Cyanobenzyl 1H-imidazole-1-carboxylate (0.756 g, 3.33 mmole) was dissolved in DMF (6 mL). Benzylamine (0.400 mL, 3.66 mmole) and N,N-diisopropylethylamine (0.630 mL, 3.62 mmole) were added and the solution was heated to 70° C. for 20 hours. The mixture was cooled to RT and diluted with ethyl acetate (25 mL). The mixture was washed with water (3×25 mL), dried over magnesium sulfate, filtered, and concentrated. The crude residue was purified by silica gel chromatography (10-40% ethyl acetate/hexane gradient) to provide 4-cyanobenzyl benzylcarbamate as a colorless oil (0.764 g, 86%). ¹H NMR (400 MHz, CDCl₃): δ 7.63 (d, J=7.9 Hz, 2H), 7.45 (d, J=7.9 Hz, 2H), 7.38-7.22 (m, 5H), 5.17 (s, 3H), 4.39 (d, J=6.0 Hz, 2H).

### 4-Carbamothioylbenzyl benzylcarbamate (30.3)

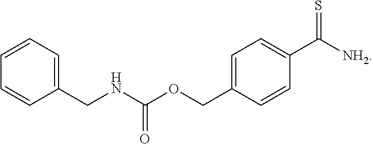 [SMILES: NC(=S)c1ccc(COC(=O)NCc2ccccc2)cc1]

4-Cyanobenzyl benzylcarbamate (0.764 g, 2.87 mmole) was dissolved in pyridine (3 mL). Triethylamine (0.450 mL, 3.21 mmole) and ammonium sulfide (40% aqueous solution, 0.590 mL, 3.45 mmole) were added and the mixture was heated to 50° C. for 4 hours, then was cooled to RT and concentrated. The residue was suspended in ethyl acetate (25 mL) and washed with 1N aq. HCl (2×25 mL). The slurry was filtered to provide a yellow solid. The filtrate was dried over magnesium sulfate, filtered, and concentrated to provide a yellow solid. The two solids were combined to provide 4-carbamothioylbenzyl benzylcarbamate as a yellow solid (0.707 g, 82%). ¹H NMR (400 MHz, DMSO-d₆): δ 9.87 (s, 1H), 9.49 (s, 1H), 7.93-7.81 (m, 3H), 7.37 (d, J=8.1 Hz, 2H), 7.35-7.28 (m, 2H), 7.25 (dt, J=8.3, 2.3 Hz, 3H), 5.08 (s, 2H), 4.21 (d, J=6.2 Hz, 2H).

### Ethyl 2-(4-(((benzylcarbamoyl)oxy)methyl)phenyl)thiazole-4-carboxylate (30.4)

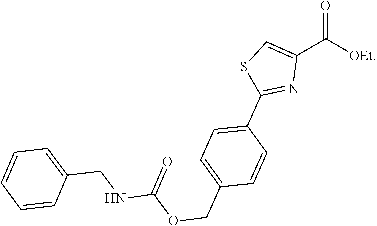 [SMILES: CCOC(=O)c1csc(-c2ccc(COC(=O)NCc3ccccc3)cc2)n1]

4-Carbamothioylbenzyl benzylcarbamate (0.707 g, 2.35 mmole) was dissolved in ethanol (12 mL). Ethyl bromopyruvate (0.360 mL, 2.87 mmole) was added and the mixture was heated to 80° C. for 3 hours, then was cooled to RT and concentrated. The residue was suspended in ethyl acetate (25 mL) and washed sequentially with sat. aq. sodium bicarbonate (2×25 mL) and water (1×25 mL). The combined aqueous washes were extracted with ethyl acetate (1×15 mL). The combined organics were dried over magnesium sulfate, filtered, and concentrated to provide ethyl 2-(4-(((benzylcarbamoyl)oxy)methyl)phenyl)thiazole-4-carboxylate as a yellow solid (0.935 g, 100%). ¹H NMR (400 MHz, CDCl₃): δ 8.15 (s, 1H), 7.99 (d, J=7.9 Hz, 2H), 7.44 (t, J=5.6 Hz, 2H), 7.38-7.27 (m, 5H), 5.18 (s, 3H), 4.45 (q, J=7.1 Hz, 2H), 4.41-4.34 (m, 2H), 1.43 (t, J=7.2 Hz, 3H).

### Methyl N-(2-(4-(((benzylcarbamoyl)oxy)methyl)phenyl)thiazole-4-carbonyl)-O-(tert-butyldimethylsilyl)-L-serinate (30.5)

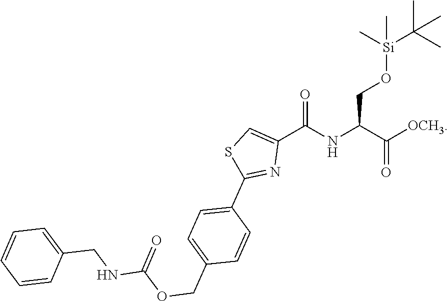 [SMILES: COC(=O)[C@H](CO[Si](C)(C)C(C)(C)C)NC(=O)c1csc(-c2ccc(COC(=O)NCc3ccccc3)cc2)n1]

Using the procedure described in Example 19.3, ethyl 2-(4-(((benzylcarbamoyl)oxy)methyl)phenyl)thiazole-4-carboxylate (0.935 g, 2.36 mmole) was converted to methyl N-(2-(4-(((benzylcarbamoyl)oxy)methyl)phenyl)thiazole-4-carbonyl)-O-(tert-butyldimethylsilyl)-L-serinate as a yellow oil (0.740 g, 54%). ¹H NMR (400 MHz, CDCl₃): δ 8.22 (d, J=8.7 Hz, 1H), 8.10 (s, 1H), 7.95 (d, J=7.9 Hz, 2H), 7.44 (d, J=7.9 Hz, 2H), 7.38-7.27 (m, 5H), 5.21 (s, 1H), 5.19 (s, 2H), 4.86 (dt, J=8.7, 3.0 Hz, 1H), 4.41 (d, J=6.0 Hz, 2H), 4.21 (dd, J=10.0, 2.6 Hz, 1H), 3.95 (dd, J=10.0, 3.3 Hz, 1H), 3.79 (s, 3H), 0.92 (s, 9H), 0.08 (s, 3H), 0.06 (s, 3H).

### Methyl O-acetyl-N—(N-(2-(4-(((benzylcarbamoyl)oxy)methyl)phenyl)thiazole-4-carbonyl)-O-(tert-butyldimethylsilyl)-L-seryl)-L-serinate (30.6)

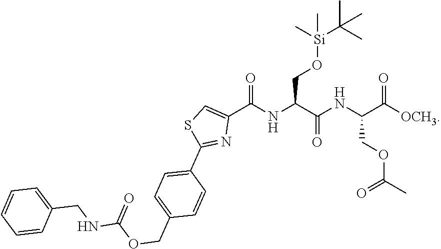 [SMILES: COC(=O)[C@H](COC(C)=O)NC(=O)[C@H](CO[Si](C)(C)C(C)(C)C)NC(=O)c1csc(-c2ccc(COC(=O)NCc3ccccc3)cc2)n1]

Using the procedure described in Example 15.6, methyl N-(2-(4-(((benzylcarbamoyl)oxy)methyl)phenyl)thiazole-4-carbonyl)-O-(tert-butyldimethylsilyl)-L-serinate (0.740 g, 1.27 mmole) was converted to methyl O-acetyl-N—(N-(2-(4-(((benzylcarbamoyl)oxy)methyl)phenyl)thiazole-4-carbonyl)-O-(tert-butyldimethylsilyl)-L-seryl)-L-serinate as a pale yellow gel (0.535 g, 59%). ¹H NMR (400 MHz, CDCl₃): δ 8.26 (d, J=7.1 Hz, 1H), 8.12 (d, J=2.5 Hz, 1H), 8.00-7.91 (m, 2H), 7.45 (t, J=8.6 Hz, 3H), 7.40-7.27 (m, 5H), 5.19 (s, 2H), 5.13 (s, 1H), 4.90 (tt, J=7.4, 3.8 Hz, 1H), 4.67 (td, J=7.4, 3.8 Hz, 1H), 4.49 (ddd, J=11.0, 7.0, 4.0 Hz, 1H), 4.45-4.37 (m, 2H), 4.34 (dd, J=11.4, 3.6 Hz, 1H), 4.23 (dt, J=9.9, 3.5 Hz, 1H), 3.85-3.78 (m, 1H), 3.77 (d, J=4.2 Hz, 3H), 2.02 (s, 3H), 0.96 (s, 9H), 0.17 (s, 3H), 0.16 (s, 3H).

### Methyl2-(2-(2-(4-(((benzylcarbamoyl)oxy)methyl)phenyl)thiazole-4-carboxamido)acrylamido)acrylate (30)

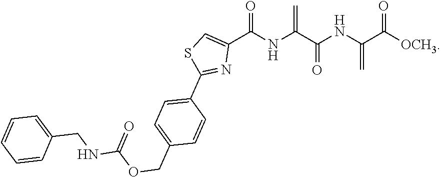 [SMILES: C=C(NC(=O)c1csc(-c2ccc(COC(=O)NCc3ccccc3)cc2)n1)C(=O)NC(=C)C(=O)OC]

Using the procedure described for Compound 15, methyl O-acetyl-N—(N-(2-(4-(((benzylcarbamoyl)oxy)methyl)phenyl)thiazole-4-carbonyl)-O-(tert-butyldimethylsilyl)-L-seryl)-L-serinate (0.535 g, 0.750 mmole) was converted to methyl 2-(2-(2-(4-(((benzylcarbamoyl)oxy)methyl)phenyl)thiazole-4-carboxamido)acrylamido)acrylate as a white solid (0.079 g, 20%). ¹H NMR (400 MHz, CDCl₃): δ 10.02 (s, 1H), 8.55 (s, 1H), 8.15 (s, 1H), 8.00 (d, J=8.0 Hz, 2H), 7.46 (d, J=7.9 Hz, 2H), 7.40-7.28 (m, 5H), 6.78 (d, J=2.2 Hz, 1H), 6.71 (s, 1H), 6.03 (d, J=1.3 Hz, 1H), 5.50 (t, J=1.9 Hz, 1H), 5.19 (s, 2H), 5.11 (s, 1H), 4.41 (d, J=6.0 Hz, 2H), 3.90 (s, 3H).

### Compound 31: Methyl (R)-2-(2-(2-(3-(((2-methoxyethoxy)carbonyl)amino)piperidin-1-yl)thiazole-4-carboxamido)acrylamido)acrylate

### Ethyl (R)-2-(3-((tert-butoxycarbonyl)amino)piperidin-1-yl)thiazole-4-carboxylate (31.1)

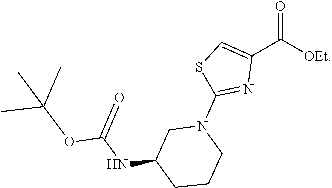 [SMILES: CCOC(=O)c1csc(N2CCC[C@@H](NC(=O)OC(C)(C)C)C2)n1]

Tert-butyl (R)-piperidin-3-ylcarbamate was dissolved in DMA. Ethyl 2-bromothiazole-4-carboxylte and triethylamine were added and the mixture was heated to 80° C. for 20 hours, then was cooled to RT and diluted with ethyl acetate (25 mL). The mixture was washed with water (3×25 mL) and the organics were dried over magnesium sulfate, filtered, and concentrated. The crude residue was purified by silica gel chromatography to provide ethyl (R)-2-(3-((tert-butoxycarbonyl)amino)piperidin-1-yl)thiazole-4-carboxylate as a white solid (1.399 g, 79%). ¹H NMR (400 MHz, CDCl₃) δ 7.44 (s, 1H), 4.73 (s, 1H), 4.35 (q, J=7.1 Hz, 2H), 3.80 (s, 1H), 3.71 (d, J=11.9 Hz, 1H), 3.62 (s, 1H), 3.45 (s, 1H), 3.30 (d, J=9.1 Hz, 1H), 1.97-1.87 (m, 1H), 1.81 (ddt, J=10.8, 7.2, 3.5 Hz, 1H), 1.76-1.63 (m, 1H), 1.60 (s, 1H), 1.50 (d, J=7.7 Hz, 9H), 1.37 (t, J=7.1 Hz, 3H).

### Ethyl (R)-2-(3-(((2-methoxyethoxy)carbonyl)amino)piperidin-1-yl)thiazole-4-carboxylate (31.2)

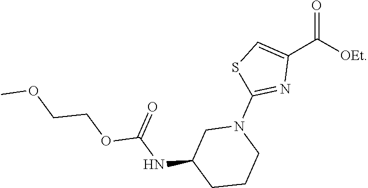 [SMILES: CCOC(=O)c1csc(N2CCC[C@@H](NC(=O)OCCOC)C2)n1]

Ethyl (R)-2-(3-((tert-butoxycarbonyl)amino)piperidin-1-yl)thiazole-4-carboxylate was dissolved in DCM. HCl (4M solution in 1,4-dioxane) was added and the mixture was stirred at RT for 2 hours and was concentrated. The residue was dissolved in DMF. 2-Methoxyethyl 1H-imidazole-1-carboxylate and N,N-diisopropylethylamine were added and the mixture was heated to 70° C. for 3 hours, then was cooled to RT and diluted with ethyl acetate (40 mL). The mixture was washed with water (3×25 mL) and the organics were dried over magnesium sulfate, filtered, and concentrated. The crude residue was purified by silica gel chromatography to provide ethyl (R)-2-(3-(((2-methoxyethoxy)carbonyl)amino)piperidin-1-yl)thiazole-4-carboxylate as a white solid (0.613 g, 44%). ¹H NMR (400 MHz, CDCl₃) δ 7.45 (s, 1H), 4.99 (d, J=7.7 Hz, 1H), 4.35 (q, J=7.1 Hz, 2H), 4.23 (t, J=4.6 Hz, 2H), 3.93-3.78 (m, 1H), 3.77-3.67 (m, 1H), 3.67-3.61 (m, 1H), 3.61-3.53 (m, 2H), 3.46-3.42 (m, 1H), 3.39 (s, 3H), 3.32 (dd, J=12.7, 7.1 Hz, 1H), 1.90 (qd, J=7.3, 3.5 Hz, 1H), 1.83 (ddt, J=14.4, 7.3, 3.6 Hz, 1H), 1.76-1.66 (m, 1H), 1.66-1.55 (m, 1H), 1.37 (t, J=7.1 Hz, 3H).

### Methyl O-(tert-butyldimethylsilyl)-N-(2-((R)-3-(((2-methoxyethoxy)carbonyl)amino)piperidin-1-yl)thiazole-4-carbonyl)-L-serinate (31.3)

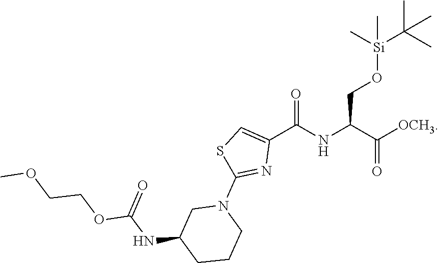 [SMILES: COCCOC(=O)N[C@@H]1CCCN(c2nc(C(=O)N[C@@H](CO[Si](C)(C)C(C)(C)C)C(=O)OC)cs2)C1]

Using the procedure described in Example 19.3, ethyl (R)-2-(3-(((2-methoxyethoxy)carbonyl)amino)piperidin-1-yl)thiazole-4-carboxylate (0.613 g, 1.72 mmole) was converted to methyl O-(tert-butyldimethylsilyl)-N-(2-((R)-3-(((2-methoxyethoxy)carbonyl)amino)piperidin-1-yl)thiazole-4-carbonyl)-L-serinate as a yellow oil (0.397 g, 42%). ¹H NMR (400 MHz, CDCl₃) δ 7.93 (d, J=8.7 Hz, 1H), 7.39 (s, 1H), 4.99 (d, J=8.0 Hz, 1H), 4.83-4.73 (m, 1H), 4.23 (q, J=3.8 Hz, 2H), 4.16 (dd, J=10.0, 2.7 Hz, 1H), 3.93-3.83 (m, 2H), 3.76 (s, 3H), 3.68 (dd, J=12.6, 3.7 Hz, 1H), 3.59 (t, J=4.7 Hz, 2H), 3.54 (d, J=6.3 Hz, 1H), 3.44-3.37 (m, 1H), 3.39 (s, 3H), 3.29 (dd, J=12.7, 7.0 Hz, 1H), 1.92 (td, J =8.3, 3.8 Hz, 1H), 1.82 (dp, J=10.9, 3.6 Hz, 1H), 1.77-1.68 (m, 1H), 1.68-1.57 (m, 1H), 0.88 (s, 9H), 0.05 (s, 3H), 0.03 (s, 3H).

### Methyl O-acetyl-N—(O-(tert-butyldimethylsilyl)-N-(2-((R)-3-(((2-methoxyethoxy)carbonyl)amino)piperidin-1-yl)thiazole-4-carbonyl)-L-seryl)-L-serinate (31.4)

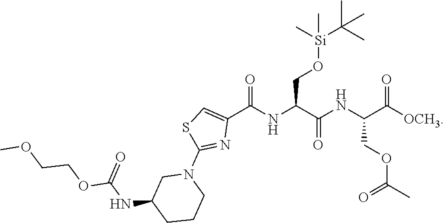 [SMILES: COCCOC(=O)N[C@@H]1CCCN(c2nc(C(=O)N[C@@H](CO[Si](C)(C)C(C)(C)C)C(=O)N[C@@H](COC(C)=O)C(=O)OC)cs2)C1]

Using the procedure described in Example 15.6, methyl O-(tert-butyldimethylsilyl)-N-(2-((R)-3-(((2-methoxyethoxy)carbonyl)amino)piperidin-1-yl)thiazole-4-carbonyl)-L-serinate (0.397 g, 0.729 mmole) was converted to methyl O-acetyl-N—(O-(tert-butyldimethylsilyl)-N-(2-((R)-3-(((2-methoxyethoxy)carbonyl)amino)piperidin-1-yl)thiazole-4-carbonyl)-L-seryl)-L-serinate as a pale yellow gel (0.367 g, 73%). ¹H NMR (400 MHz, CDCl₃) δ 7.95 (d, J=7.2 Hz, 1H), 7.44 (d, J=7.8 Hz, 1H), 7.40 (s, 1H), 4.98 (t, J=10.1 Hz, 1H), 4.88 (dt, J=7.8, 3.8 Hz, 1H), 4.59 (td, J=7.3, 3.7 Hz, 1H), 4.47 (ddd, J=12.0, 8.1, 4.0 Hz, 1H), 4.31 (dd, J=11.4, 3.7 Hz, 1H), 4.23 (d, J=4.4 Hz, 2H), 4.18 (ddd, J=9.8, 3.7, 1.6 Hz, 1H), 3.91-3.81 (m, 1H), 3.76 (s, 3H), 3.75-3.70 (m, 1H), 3.62-3.52 (m, 3H), 3.44-3.39 (m, 1H), 3.39 (s, 3H), 3.31-3.14 (m, 1H), 2.05 (s, 3H), 1.98-1.87 (m, 1H), 1.83 (ddt, J=14.1, 7.0, 3.3 Hz, 1H), 1.72 (dq, J=9.6, 5.0, 4.5 Hz, 1H), 0.92 (s, 9H), 0.14 (s, 3H), 0.13 (s, 3H).

### Methyl (R)-2-(2-(2-(3-(((2-methoxyethoxy)carbonyl)amino)piperidin-1-yl)thiazole-4-carboxamido)acrylamido)acrylate (31)

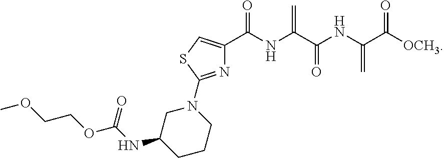 [SMILES: C=C(NC(=O)c1csc(N2CCC[C@@H](NC(=O)OCCOC)C2)n1)C(=O)NC(=C)C(=O)OC]

Using the procedure described for Compound 15, methyl O-acetyl-N—(O-(tert-butyldimethylsilyl)-N-(2-((R)-3-(((2-methoxyethoxy)carbonyl)amino)piperidin-1-yl)thiazole-4-carbonyl)-L-seryl)-L-serinate (0.361 g, 0.536 mmole) was converted to methyl (R)-2-(2-(2-(3-(((2-methoxyethoxy)carbonyl)amino)piperidin-1-yl)thiazole-4-carboxamido)acrylamido)acrylate as a colorless gel (0.116 g, 45%). ¹H NMR (400 MHz, CDCl₃) δ 9.69 (s, 1H), 8.51 (s, 1H), 7.44 (s, 1H), 6.71 (d, J=2.1 Hz, 1H), 6.67 (s, 1H), 6.00 (d, J=1.3 Hz, 1H), 5.44 (t, J=1.8 Hz, 1H), 5.00 (d, J=7.9 Hz, 1H), 4.24 (dd, J=6.9, 2.6 Hz, 2H), 3.92-3.83 (m, 1H), 3.89 (s, 3H), 3.67 (d, J=15.8 Hz, 1H), 3.58 (t, J=4.6 Hz, 2H), 3.44 (s, 1H), 3.39 (s, 3H), 3.35 (t, J=6.3 Hz, 1H), 1.89 (d, J=3.0 Hz, 1H), 1.84 (dt, J=7.1, 3.1 Hz, 1H), 1.78-1.62 (m, 3H).

### Compound 32: Methyl 2-(2-(2-(4-((((3-methoxypropyl)carbamoyl)oxy)methyl)piperidin-1-yl)thiazole-4-carboxamido)acrylamido)acrylate

### Tert-butyl 4-(((1H-imidazole-1-carbonyl)oxy)methyl)piperidine-1-carboxylate (32.1)

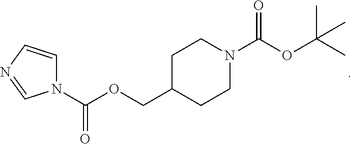 [SMILES: CC(C)(C)OC(=O)N1CCC(COC(=O)n2ccnc2)CC1]

Using the procedure described in Example 27.1, tert-butyl 4-(hydroxymethyl)piperidine-1-carboxylate (2.007 g, 9.32 mmole) was converted to tert-butyl 4-(((1H-imidazole-1-carbonyl)oxy)methyl)piperidine-1-carboxylate (3.389 g) as a pale yellow oil. ¹H NMR (400 MHz, CDCl₃) δ 8.14 (t, J=1.1 Hz, 1H), 7.42 (t, J=1.5 Hz, 1H), 7.08 (dd, J=1.6, 0.9 Hz, 1H), 4.29 (d, J=6.6 Hz, 2H), 4.24-4.13 (m, 2H), 2.74 (t, J=12.8 Hz, 2H), 2.03-1.93 (m, 1H), 1.81-1.70 (m, 2H), 1.46 (s, 9H), 1.34-1.26 (m, 2H)

### Tert-butyl 4-((((3-methoxypropyl)carbamoyl)oxy)methyl)piperidine-1-carboxylate (32.2)

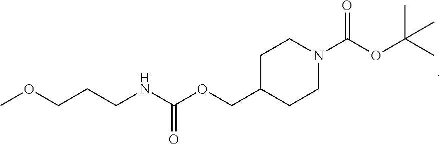 [SMILES: COCCCNC(=O)OCC1CCN(C(=O)OC(C)(C)C)CC1]

Tert-butyl 4-(((1H-imidazole-1-carbonyl)oxy)methyl)piperidine-1-carboxylate (0.994 g, 3.21 mmole) was dissolved in DMF (6 mL). 3-Methoxypropan-1-amine (0.365 mL, 3.58 mmole) and N,N-diisopropylethylamine (0.680 mL, 3.90 mmole) were added and the mixture was heated to 70° C. for 18 hours, then was cooled to RT and diluted with ethyl acetate (25 mL). The mixture was washed with water (3×25 mL) and the organics were dried over magnesium sulfate, filtered, and concentrated. The crude residue was purified by silica gel chromatography to provide tert-butyl 4-((((3-methoxypropyl)carbamoyl)oxy)methyl)piperidine-1-carboxylate as a colorless oil (0.779 g, 73%). ¹H NMR (400 MHz, CDCl₃) δ 5.10 (s, 1H), 4.17-4.05 (m, 2H), 3.91 (d, J=6.5 Hz, 2H), 3.46 (t, J=5.8 Hz, 2H), 3.34 (s, 3H), 3.28 (q, J=6.3 Hz, 2H), 2.77-2.61 (m, 2H), 1.83-1.73 (m, 3H), 1.70 (s, 2H), 1.45 (s, 9H), 1.22-1.10 (m, 2H).

### Ethyl 2-(4-((((3-methoxypropyl)carbamoyl)oxy)methyl)piperidin-1-yl)thiazole-4-carboxylate (32.3)

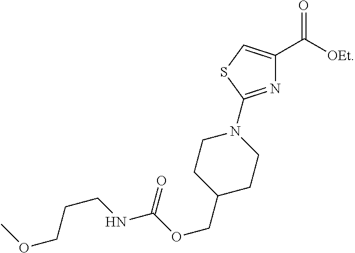 [SMILES: CCOC(=O)c1csc(N2CCC(COC(=O)NCCCOC)CC2)n1]

Tert-butyl 4-((((3-methoxypropyl)carbamoyl)oxy)methyl)piperidine-1-carboxylate (0.779 g, 2.36 mmole) was dissolved in DCM (5 mL). HCl (4M solution in 1,4-dioxane, 2.40 mL, 9.60 mmole) was added and the mixture was stirred at RT for 2 hours, then was concentrated. The residue was dissolved in DMA (3 mL). Ethyl 2-bromothiazole-4-carboxylate (0.564 g, 2.39 mmole) and triethylamine (0.660 mL, 4.71 mmole) were added and the mixture was heated to 80° C. for 20 hours, then was cooled to RT and diluted with ethyl acetate (25 mL). The mixture was washed with water (3×25 mL) and the organics were dried over magnesium sulfate, filtered, and concentrated. The crude residue was purified by silica gel chromatography to provide ethyl 2-(4-((((3-methoxypropyl)carbamoyl)oxy)methyl)piperidin-1-yl)thiazole-4-carboxylate as a pale tan solid (0.460 g, 51%). ¹H NMR (400 MHz, CDCl₃) δ 7.42 (s, 1H), 5.08 (s, 1H), 4.35 (q, J=7.1 Hz, 2H), 4.07 (dt, J=12.7, 3.4 Hz, 2H), 3.95 (d, J=6.2 Hz, 2H), 3.46 (t, J=5.8 Hz, 2H), 3.34 (s, 3H), 3.29 (q, J=6.2 Hz, 2H), 3.03 (td, J=12.6, 2.8 Hz, 2H), 1.95-1.67 (m, 5H), 1.37 (t, J=7.1 Hz, 5H).

### Methyl O-(tert-butyldimethylsilyl)-N-(2-(4-((((3-methoxypropyl)carbamoyl)oxy)methyl)piperidin-1-yl)thiazole-4-carbonyl)-L-serinate (32.4)

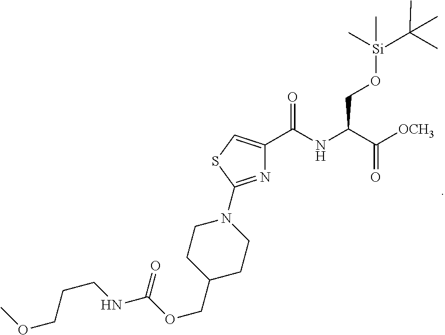 [SMILES: COCCCNC(=O)OCC1CCN(c2nc(C(=O)N[C@@H](CO[Si](C)(C)C(C)(C)C)C(=O)OC)cs2)CC1]

Using the procedure described in Example 19.3, ethyl 2-(4-((((3-methoxypropyl)carbamoyl)oxy)methyl)piperidin-1-yl)thiazole-4-carboxylate (0.460 g, 1.19 mmole) was converted to methyl O-(tert-butyldimethylsilyl)-N-(2-(4-((((3-methoxypropyl)carbamoyl)oxy)methyl)piperidin-1-yl)thiazole-4-carbonyl)-L-serinate as a pale yellow oil (0.447 g, 66%). ¹H NMR (400 MHz, CDCl₃) δ 7.97 (d, J=8.8 Hz, 1H), 7.36 (s, 1H), 5.08 (s, 1H), 4.82-4.74 (m, 1H), 4.16 (dd, J=10.0, 2.6 Hz, 1H), 4.02 (d, J=12.8 Hz, 2H), 3.96 (d, J=6.2 Hz, 2H), 3.88 (dd, J=10.0, 3.4 Hz, 1H), 3.76 (s, 3H), 3.47 (t, J=5.8 Hz, 2H), 3.34 (s, 3H), 3.29 (t, J=6.3 Hz, 2H), 3.01 (tdd, J=12.4, 5.5, 2.8 Hz, 2H), 1.96-1.86 (m, 1H), 1.85-1.74 (m, 4H), 1.47-1.31 (m, 2H), 0.88 (s, 9H), 0.06 (s, 3H), 0.03 (s, 3H).

### Methyl O-acetyl-N—(O-(tert-butyldimethylsilyl)-N-(2-(4-((((3-methoxypropyl)carbamoyl)oxy)methyl)piperidin-1-yl)thiazole-4-carbonyl)-L-seryl)-L-serinate (32.5)

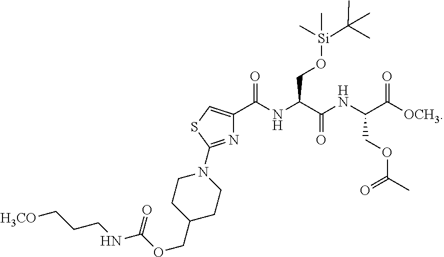 [SMILES: COCCCNC(=O)OCC1CCN(c2nc(C(=O)N[C@@H](CO[Si](C)(C)C(C)(C)C)C(=O)N[C@@H](COC(C)=O)C(=O)OC)cs2)CC1]

Using the procedure described in Example 15.6, methyl O-(tert-butyldimethylsilyl)-N-(2-(4-((((3-methoxypropyl)carbamoyl)oxy)methyl)piperidin-1-yl)thiazole-4-carbonyl)-L-serinate (0.447 g, 0.780 mmole) was converted to methyl O-acetyl-N—(O-(tert-butyldimethylsilyl)-N-(2-(4-((((3-methoxypropyl)carbamoyl)oxy)methyl)piperidin-1-yl)thiazole-4-carbonyl)-L-seryl)-L-serinate as a pale yellow oil (0.366 g, 67%). ¹H NMR (400 MHz, CDCl₃) δ 8.00 (d, J=7.1 Hz, 1H), 7.43 (d, J=7.8 Hz, 1H), 7.38 (s, 1H), 5.09 (s, 1H), 4.88 (dt, J=7.7, 3.8 Hz, 1H), 4.58 (ddd, J=7.2, 5.8, 3.6 Hz, 1H), 4.47 (dt, J=11.4, 4.2 Hz, 1H), 4.31 (dd, J=11.4, 3.7 Hz, 1H), 4.19 (ddd, J=9.8, 3.6, 2.2 Hz, 1H), 4.02 (d, J=12.9 Hz, 2H), 3.96 (d, J=6.2 Hz, 2H), 3.76 (s, 3H), 3.75-3.70 (m, 1H), 3.47 (t, J=5.8 Hz, 2H), 3.34 (s, 3H), 3.29 (t, J=6.2 Hz, 2H), 3.00 (ddd, J=15.5, 7.9, 3.2 Hz, 2H), 2.05 (s, 3H), 1.87 (d, J=10.7 Hz, 1H), 1.85-1.73 (m, 4H), 1.47-1.32 (m, 2H), 0.93 (s, 9H), 0.14 (s, 3H), 0.13 (s, 3H).

### Methyl 2-(2-(2-(4-((((3-methoxypropyl)carbamoyl)oxy)methyl)piperidin-1-yl)thiazole-4-carboxamido)acrylamido)acrylate (32)

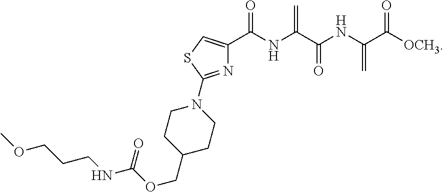 [SMILES: C=C(NC(=O)c1csc(N2CCC(COC(=O)NCCCOC)CC2)n1)C(=O)NC(=C)C(=O)OC]

Using the procedure described for Compound 15, methyl O-acetyl-N—(O-(tert-butyldimethylsilyl)-N-(2-(4-((((3-methoxypropyl)carbamoyl)oxy)methyl)piperidin-1-yl)thiazole-4-carbonyl)-L-seryl)-L-serinate (0.366 g, 0.521 mmole) was converted to methyl 2-(2-(2-(4-((((3-methoxypropyl)carbamoyl)oxy)methyl)piperidin-1-yl)thiazole-4-carboxamido)acrylamido)acrylate as a white solid (0.098 g, 37%). ¹H NMR (400 MHz, CDCl₃) δ 9.73 (s, 1H), 8.51 (s, 1H), 7.41 (s, 1H), 6.71 (d, J=2.1 Hz, 1H), 6.66 (s, 1H), 6.00 (d, J=1.3 Hz, 1H), 5.43 (t, J=1.8 Hz, 1H), 5.07 (s, 1H), 4.05 (dd, J=13.0, 3.7 Hz, 2H), 3.97 (d, J=6.2 Hz, 2H), 3.89 (s, 3H), 3.47 (t, J=5.8 Hz, 2H), 3.34 (s, 3H), 3.30 (q, J=6.2 Hz, 2H), 3.03 (td, J =12.7, 2.8 Hz, 2H), 1.90 (d, J=6.3 Hz, 1H), 1.84 (d, J=13.3 Hz, 2H), 1.79 (q, J=6.2 Hz, 2H), 1.47-1.32 (m, 2H).

### Compound 33: Methyl 2-(2-(2-(4-(((2-methoxyethoxy)carbonyl)amino)piperidin-1-yl)thiazole-4-carboxamido)acrylamido)acrylate

### Ethyl 2-(4-(((2-methoxyethoxy)carbonyl)amino)piperidin-1-yl)thiazole-4-carboxylate (33.1)

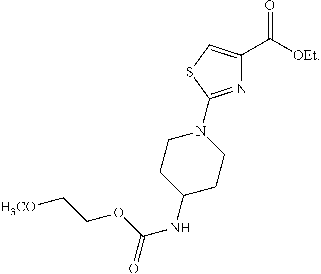 [SMILES: CCOC(=O)c1csc(N2CCC(NC(=O)OCCOC)CC2)n1]

Using the procedure described in Example 27.2, ethyl 2-(4-((tert-butoxycarbonyl)amino)piperidin-1-yl)thiazole-4-carboxylate (Example 12.1, 0.956 g, 2.69 mmole) was converted to ethyl 2-(4-(((2-methoxyethoxy)carbonyl)amino)piperidin-1-yl)thiazole-4-carboxylate (0.512 g, 53%) as a white solid. ¹H NMR (400 MHz, CDCl₃) δ 7.44 (s, 1H), 4.76 (d, J=7.5 Hz, 1H), 4.35 (q, J=7.1 Hz, 2H), 4.23 (t, J=4.5 Hz, 2H), 4.04-3.94 (m, 2H), 3.73 (td, J=10.9, 9.2, 5.1 Hz, 1H), 3.62-3.55 (m, 2H), 3.40 (s, 3H), 3.17 (ddd, J=13.3, 11.5, 3.0 Hz, 2H), 2.06 (ddt, J=12.6, 5.0, 2.3 Hz, 2H), 1.51 (dtd, J=12.8, 11.2, 4.3 Hz, 2H), 1.37 (t, J=7.1 Hz, 3H).

### Methyl O-(tert-butyldimethylsilyl)-N-(2-(4-(((2-methoxyethoxy)carbonyl)amino)piperidin-1-yl)thiazole-4-carbonyl)-L-serinate (33.2)

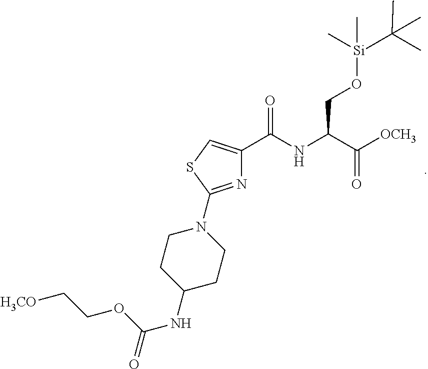 [SMILES: COCCOC(=O)NC1CCN(c2nc(C(=O)N[C@@H](CO[Si](C)(C)C(C)(C)C)C(=O)OC)cs2)CC1]

Using the procedure described in Example 19.3, ethyl 2-(4-(((2-methoxyethoxy)carbonyl)amino)piperidin-1-yl)thiazole-4-carboxylate (0.512 g, 1.43 mmole) was converted to methyl O-(tert-butyldimethylsilyl)-N-(2-(4-(((2-methoxyethoxy)carbonyl)amino)piperidin-1-yl)thiazole-4-carbonyl)-L-serinate as a colorless oil (0.398 g, 51%). ¹H NMR (400 MHz, CDCl₃) δ 7.95 (d, J=8.8 Hz, 1H), 7.38 (s, 1H), 4.82-4.74 (m, 2H), 4.27-4.20 (m, 2H), 4.20-4.14 (m, 1H), 4.00-3.91 (m, 2H), 3.88 (dd, J=10.0, 3.4 Hz, 1H), 3.76 (s, 4H), 3.63-3.55 (m, 2H), 3.40 (s, 3H), 3.15 (dddd, J=13.3, 11.5, 7.2, 3.0 Hz, 2H), 2.09-2.00 (m, 2H), 1.59-1.45 (m, 2H), 0.88 (s, 9H), 0.05 (s, 3H), 0.03 (s, 3H).

### Methyl O-acetyl-N—(O-(tert-butyldimethylsilyl)-N-(2-(4-(((2-methoxyethoxy)carbonyl)amino)piperidin-1-yl)thiazole-4-carbonyl)-L-seryl)-L-serinate (33.3)

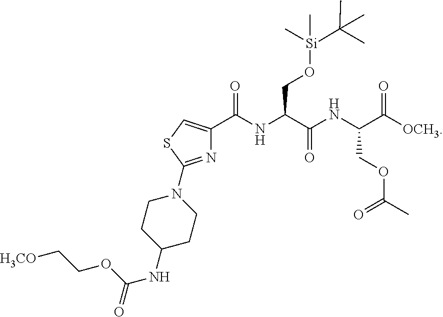 [SMILES: COCCOC(=O)NC1CCN(c2nc(C(=O)N[C@@H](CO[Si](C)(C)C(C)(C)C)C(=O)N[C@@H](COC(C)=O)C(=O)OC)cs2)CC1]

Using the procedure described in Example 15.6, methyl O-(tert-butyldimethylsilyl)-N-(2-(4-(((2-methoxyethoxy)carbonyl)amino)piperidin-1-yl)thiazole-4-carbonyl)-L-serinate (0.398 g, 0.731 mmole) was converted to methyl O-acetyl-N—(O-(tert-butyldimethylsilyl)-N-(2-(4-(((2-methoxyethoxy)carbonyl)amino)piperidin-1-yl)thiazole-4-carbonyl)-L-seryl)-L-serinate as a pale yellow oil (0.366 g, 74%). ¹H NMR (400 MHz, CDCl₃) δ 7.98 (d, J=7.1 Hz, 1H), 7.43 (d, J=7.8 Hz, 1H), 7.39 (s, 1H), 4.87 (dt, J=7.7, 3.8 Hz, 1H), 4.77 (d, J=7.8 Hz, 1H), 4.63-4.54 (m, 1H), 4.47 (ddd, J=11.3, 9.5, 4.0 Hz, 1H), 4.31 (dd, J=11.4, 3.7 Hz, 1H), 4.27-4.21 (m, 2H), 4.21-4.17 (m, 1H), 3.94 (d, J=10.4 Hz, 2H), 3.80-3.69 (m, 2H), 3.76 (s, 3H), 3.59 (dd, J=5.5, 3.6 Hz, 2H), 3.40 (s, 3H), 3.22-3.09 (m, 2H), 2.11-2.05 (m, 2H), 2.05 (s, 3H), 1.50 (dd, J=14.1, 9.5 Hz, 2H), 0.92 (s, 9H), 0.14 (s, 3H), 0.13 (s, 3H).

### Methyl 2-(2-(2-(4-(((2-methoxyethoxy)carbonyl)amino)piperidin-1-yl)thiazole-4-carboxamido)acrylamido)acrylate (33)

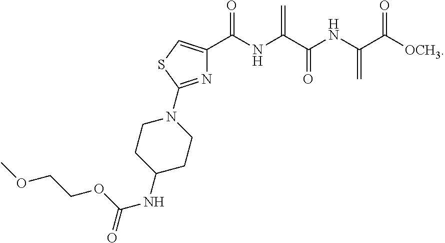 [SMILES: C=C(NC(=O)c1csc(N2CCC(NC(=O)OCCOC)CC2)n1)C(=O)NC(=C)C(=O)OC]

Using the procedure described for Compound 15, methyl O-acetyl-N—(O-(tert-butyldimethylsilyl)-N-(2-(4-(((2-methoxyethoxy)carbonyl)amino)piperidin-1-yl)thiazole-4-carbonyl)-L-seryl)-L-serinate (0.366 g, 0.543 mmole) was converted to methyl 2-(2-(2-(4-(((2-methoxyethoxy)carbonyl)amino)piperidin-1-yl)thiazole-4-carboxamido)acrylamido)acrylate as a white solid (0.124 g, 47%). ¹H NMR (400 MHz, CDCl₃) δ 9.71 (s, 1H), 8.51 (s, 1H), 7.43 (s, 1H), 6.71 (d, J=2.1 Hz, 1H), 6.66 (s, 1H), 6.00 (d, J=1.3 Hz, 1H), 5.43 (t, J=1.9 Hz, 1H), 4.78 (d, J=7.8 Hz, 1H), 4.24 (t, J=4.6 Hz, 2H), 3.98 (dt, J=13.6, 3.9 Hz, 2H), 3.89 (s, 3H), 3.83-3.69 (m, 1H), 3.64-3.56 (m, 2H), 3.40 (s, 3H), 3.18 (ddd, J=13.2, 11.5, 3.0 Hz, 2H), 2.15-2.05 (m, 2H), 1.53 (dtd, J=12.8, 11.1, 4.3 Hz, 2H).

### Compound 34: Methyl 2-(2-(2-(4-(2-(2-methoxyethoxy)acetamido)piperidin-1-yl)thiazole-4-carboxamido)acrylamido)acrylate

### Methyl (2-(4-((tert-butoxycarbonyl)amino)piperidin-1-yl)thiazole-4-carbonyl)-L-serinate (34.1)

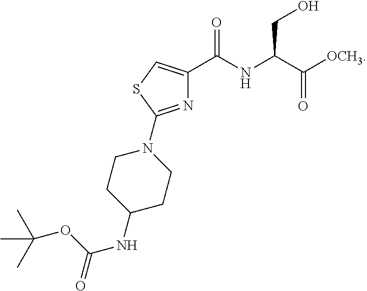 [SMILES: COC(=O)[C@H](CO)NC(=O)c1csc(N2CCC(NC(=O)OC(C)(C)C)CC2)n1]

Ethyl 2-(4-((tert-butoxycarbonyl)amino)piperidin-1-yl)thiazole-4-carboxylate (Example 12.1, 1.526 g, 4.29 mmole) was dissolved in 4/1/1 THF/methanol/water (8 mL). Lithium hydroxide monohydrate (0.356 g, 8.48 mmole) was added and the mixture was stirred at RT for 4 hours. Water (25 mL) was added and the solution was treated with 1N aq. HCl to pH=4. The mixture was extracted with ethyl acetate (3×15 mL) and the combined organics were dried over magnesium sulfate, filtered, and concentrated. The residue was suspended in DCM (8 mL). L-Serine methyl ester hydrochloride (0.795 g, 5.11 mmole) was added followed by N,N-diisopropylethylamine (1.50 mL, 8.61 mmole) and pyBOP (2.673 g, 5.14 mmole). The resulting solution was stirred at RT for 18 hours and water (25 mL) was added. The two layers were separated and the aqueous layer was extracted with DCM (3×10 mL). The combined organics were dried over magnesium sulfate, filtered, and concentrated. The crude residue was purified by silica gel chromatography to provide methyl (2-(4-((tert-butoxycarbonyl)amino)piperidin-1-yl)thiazole-4-carbonyl)-L-serinate as a white solid (1.648 g, 90%). ¹H NMR (400 MHz, CDCl₃) δ 7.99 (d, J=7.5 Hz, 1H), 7.38 (s, 1H), 4.79 (dt, J=7.6, 3.8 Hz, 1H), 4.59 (d, J=7.9 Hz, 1H), 4.08-3.99 (m, 2H), 3.94 (ddd, J=13.0, 5.5, 2.9 Hz, 2H), 3.81 (s, 3H), 3.65 (d, J=25.0 Hz, 1H), 3.13 (ddt, J=13.6, 11.2, 2.6 Hz, 2H), 2.04 (dd, J=12.9, 3.7 Hz, 2H), 1.57-1.47 (m, 2H), 1.46 (s, 9H).

### Methyl O-(tert-butyldimethylsilyl)-N-(2-(4-(2-(2-methoxyethoxy)acetamido)piperidin-1-yl)thiazole-4-carbonyl)-L-serinate (34.2)

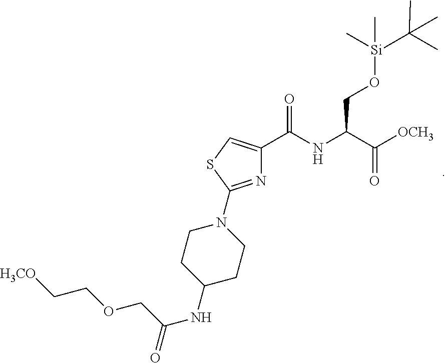 [SMILES: COCCOCC(=O)NC1CCN(c2nc(C(=O)N[C@@H](CO[Si](C)(C)C(C)(C)C)C(=O)OC)cs2)CC1]

Methyl (2-(4-((tert-butoxycarbonyl)amino)piperidin-1-yl)thiazole-4-carbonyl)-L-serinate (0.506 g, 1.18 mmole) was dissolved in DCM (2.5 mL). HCl (4M solution in 1,4-dioxane, 1.20 mL, 4.80 mmole) was added and the mixture was stirred at RT for 5 hours, then was concentrated. A solution of 2-(2-methoxyethoxy)acetic acid (0.192 g, 1.43 mmole) in DCM (2.5 mL) was added to the residue, followed by N,N-diisopropylethylamine (0.410 mL, 2.35 mmole), HOBt·H₂O (0.221 g, 1.44 mmole), and EDC·HCl (0.267 g, 1.39 mmole). The mixture was stirred at RT for 2 ½ days and water (25 mL) was added. The mixture was extracted with DCM (3×10 mL) and the combined organics were dried over magnesium sulfate, filtered, and concentrated. The residue was dissolved in DCM (2.5 mL) and imidazole (0.088 g, 1.29 mmole) and tert-butyldimethylchlorosilane (0.192 g, 1.27 mmole) were added. The mixture was stirred at RT for 18 hours and water (25 mL) was added. The mixture was extracted with DCM (3×10 mL) and the combined organics were dried over magnesium sulfate, filtered, and concentrated. The crude residue was purified by silica gel chromatography to provide methyl O-(tert-butyldimethylsilyl)-N-(2-(4-(2-(2-methoxyethoxy)acetamido)piperidin-1-yl)thiazole-4-carbonyl)-L-serinate as a thick, colorless oil (0.361 g, 55%). ¹H NMR (400 MHz, CDCl₃) δ 7.96 (d, J=8.8 Hz, 1H), 7.39 (s, 1H), 7.13 (d, J=8.0 Hz, 1H), 4.78 (ddd, J=8.8, 3.4, 2.6 Hz, 1H), 4.16 (dd, J=10.0, 2.6 Hz, 1H), 4.12-4.03 (m, 1H), 4.00 (s, 3H), 3.97-3.91 (m, 1H), 3.88 (dd, J=10.0, 3.4 Hz, 1H), 3.76 (s, 3H), 3.71-3.64 (m, 2H), 3.59-3.52 (m, 2H), 3.39 (s, 3H), 3.18 (dddd, J=13.2, 11.7, 8.7, 3.0 Hz, 2H), 2.09-1.98 (m, 2H), 1.68-1.51 (m, 2H), 0.88 (s, 9H), 0.05 (s, 3H), 0.03 (s, 3H).

### Methyl O-acetyl-N—(O-(tert-butyldimethylsilyl)-N-(2-(4-(2-(2-methoxyethoxy)acetamido)piperidin-1-yl)thiazole-4-carbonyl)-L-seryl)-L-serinate (34.3)

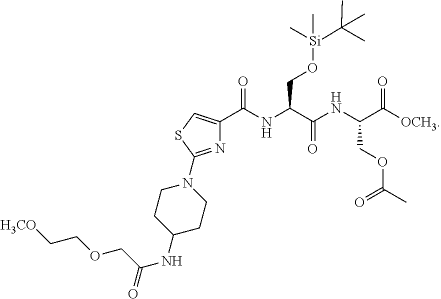 [SMILES: COCCOCC(=O)NC1CCN(c2nc(C(=O)N[C@@H](CO[Si](C)(C)C(C)(C)C)C(=O)N[C@@H](COC(C)=O)C(=O)OC)cs2)CC1]

Using the procedure described in Example 15.6, methyl O-(tert-butyldimethylsilyl)-N-(2-(4-(2-(2-methoxyethoxy)acetamido)piperidin-1-yl)thiazole-4-carbonyl)-L-serinate (0.361 g, 0.646 mmole) was converted to methyl O-acetyl-N—(O-(tert-butyldimethylsilyl)-N-(2-(4-(2-(2-methoxyethoxy)acetamido)piperidin-1-yl)thiazole-4-carbonyl)-L-seryl)-L-serinate as a colorless gel (0.267 g, 60%). ¹H NMR (400 MHz, CDCl₃) δ 7.99 (d, J=7.1 Hz, 1H), 7.44 (d, J=7.8 Hz, 1H), 7.40 (d, J=2.6 Hz, 1H), 7.13 (d, J=8.0 Hz, 1H), 4.87 (dq, J=8.3, 4.2 Hz, 1H), 4.58 (tdd, J=7.0, 3.6, 2.1 Hz, 1H), 4.47 (ddd, J=11.4, 9.0, 4.0 Hz, 1H), 4.31 (dd, J=11.4, 3.7 Hz, 1H), 4.19 (ddd, J=9.8, 3.6, 1.2 Hz, 1H), 4.09-4.02 (m, 1H), 4.02-3.91 (m, 2H), 3.99 (s, 2H), 3.76 (s, 3H), 3.75-3.71 (m, 1H), 3.71-3.65 (m, 2H), 3.60-3.51 (m, 2H), 3.39 (s, 3H), 3.18 (dddd, J=13.0, 11.5, 4.7, 2.9 Hz, 2H), 2.09-2.03 (m, 2H), 2.02 (s, 3H), 1.63-1.51 (m, 2H), 0.92 (d, J=1.0 Hz, 9H), 0.14 (s, 3H), 0.13 (s, 3H).

### Methyl 2-(2-(2-(4-(2-(2-methoxyethoxy)acetamido)piperidin-1-yl)thiazole-4-carboxamido)acrylamido)acrylate (34)

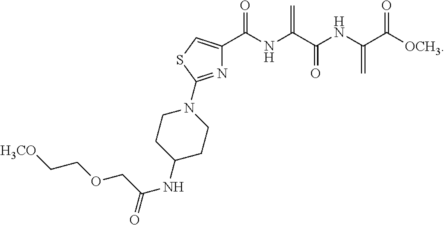 [SMILES: C=C(NC(=O)c1csc(N2CCC(NC(=O)COCCOC)CC2)n1)C(=O)NC(=C)C(=O)OC]

Using the procedure described for Compound 15, methyl O-acetyl-N—(O-(tert-butyldimethylsilyl)-N-(2-(4-(((2-methoxyethoxy)carbonyl)amino)piperidin-1-yl)thiazole-4-carbonyl)-L-seryl)-L-serinate (0.366 g, 0.543 mmole) was converted to methyl 2-(2-(2-(4-(((2-methoxyethoxy)carbonyl)amino)piperidin-1-yl)thiazole-4-carboxamido)acrylamido)acrylate as a white solid (0.124 g, 47%). ¹H NMR (400 MHz, CDCl₃) δ 9.72 (s, 1H), 8.51 (s, 1H), 7.43 (s, 1H), 7.13 (d, J=8.0 Hz, 1H), 6.72 (d, J=2.1 Hz, 1H), 6.66 (s, 1H), 6.01 (d, J=1.3 Hz, 1H), 5.44 (t, J=1.8 Hz, 1H), 4.13-4.04 (m, 1H), 4.04-3.94 (m, 2H), 3.99 (s, 2H), 3.89 (s, 3H), 3.71-3.63 (m, 2H), 3.59-3.51 (m, 2H), 3.39 (s, 3H), 3.27-3.15 (m, 2H), 2.12-2.01 (m, 2H), 1.63-1.51 (m, 2H).

### Compound 35: Methyl 2-(2-(2-(4-((2-(2-methoxyethoxy)acetamido)methyl)phenyl)thiazole-4-carboxamido)acrylamido)acrylate

### Tert-butyl (4-cyanobenzyl)carbamate (35.1)

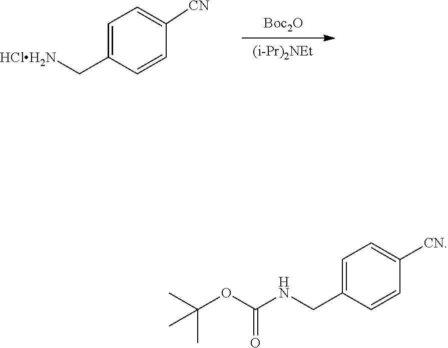

4-(Aminomethyl)benzonitrile hydrochloride (3.013 g, 17.9 mmole) was suspended in DCM (36 mL). Di-tert-butyl dicarbonate (4.100 g, 18.8 mmole) was added followed by N,N-diisopropylethylamine (6.20 mL, 35.6 mmole) and the mixture was stirred at RT for 18 hours. Water (50 mL) was added and the two layers were separated. The aqueous layer was extracted with DCM (2×15 mL) and the combined organics were dried over magnesium sulfate, filtered, and concentrated to provide tert-butyl (4-cyanobenzyl)carbamate as a white solid (4.901 g). ¹H NMR (400 MHz, CDCl₃): δ 7.66-7.58 (m, 2H), 7.39 (d, J=8.2 Hz, 2H), 5.03 (s, 1H), 4.37 (d, J=6.3 Hz, 2H), 1.46 (s, 9H)

### Tert-butyl (4-carbamothioylbenzyl)carbamate (35.2)

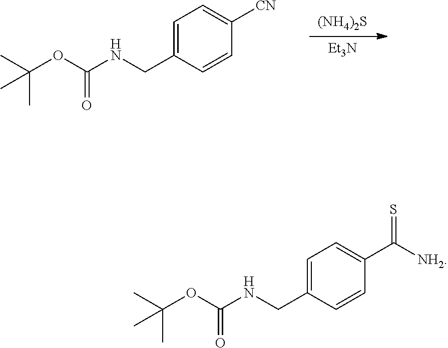 [SMILES: "Et3N" ; CC(C)(C)OC(=O)NCc1ccc(C#N)cc1 ; "(NH4)2S" ; CC(C)(C)OC(=O)NCc1ccc(C(N)=S)cc1]

Tert-butyl (4-cyanobenzyl)carbamate (4.901 g, 21.1 mmole) was dissolved in pyridine (21 mL). Triethylamine (3.30 mL, 23.5 mmole) and ammonium sulfide (40% aqueous solution, 4.40 mL, 25.8 mmole) were added and the mixture was heated to 50° C. for 4 ½ hours, then was cooled to RT and concentrated. The residue was dissolved in ethyl acetate (50 mL) and the mixture was washed sequentially with 1N aq. HCl (2×25 mL) and sat. aq. sodium chloride (1×25 mL). The organics were dried over magnesium sulfate, filtered, and concentrated to provide tert-butyl (4-carbamothioylbenzyl)carbamate as a yellow solid (5.376 g). 1H NMR (400 MHz, DMSO) δ 9.81 (s, 1H), 9.44 (s, 1H), 7.89-7.82 (m, 2H), 7.44 (q, J=6.5 Hz, 1H), 7.25 (d, J=8.3 Hz, 2H), 4.15 (d, J=6.2 Hz, 2H), 1.39 (s, 9H).

### Ethyl 2-(4-(((tert-butoxycarbonyl)amino)methyl)phenyl)thiazole-4-carboxylate (35.3)

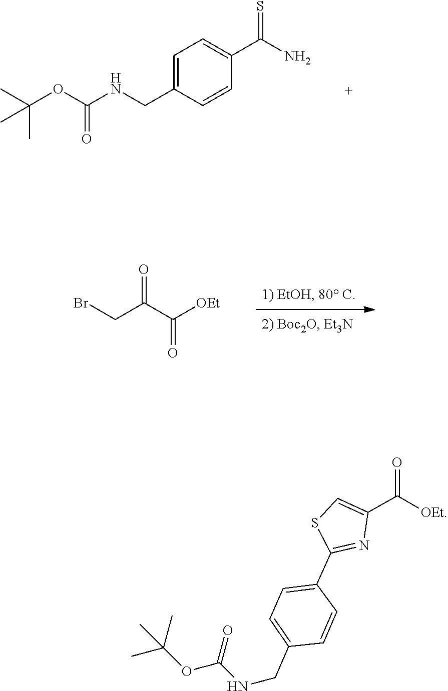 [SMILES: CC(C)(C)OC(=O)NCc1ccc(C(N)=S)cc1 ; CCOC(=O)C(=O)CBr ; CCOC(=O)c1csc(-c2ccc(CNC(=O)OC(C)(C)C)cc2)n1]

Tert-butyl (4-carbamothioylbenzyl)carbamate (5.376 g, 20.1 mmole) was dissolved in ethanol (40 mL). Ethyl bromopyruvate (2.80 mL, 22.3 mmole) was added and the mixture was heated to 80° C. for 18 hours, then was cooled to RT and concentrated. The residue was suspended in DCM (40 mL) and triethylamine (5.70 mL, 40.7 mmole) and di-tert-butyl dicarbonate (4.857 g, 22.3 mmole) were added. The mixture was stirred at RT for 18 hours and water (100 mL) was added. The two layers were separated and the aqueous layer was extracted with DCM (2×20 mL). The combined organics were dried over magnesium sulfate, filtered, and concentrated. The crude residue was purified by silica gel chromatography to provide ethyl 2-(4-(((tert-butoxycarbonyl)amino)methyl)phenyl)thiazole-4-carboxylate as an orange solid (5.003 g, 69%). ¹H NMR (400 MHz, CDCl₃): δ 8.14 (s, 1H), 8.01-7.92 (m, 2H), 7.36 (d, J=8.2 Hz, 2H), 5.02 (d, J=29.7 Hz, 1H), 4.45 (q, J=7.1 Hz, 2H), 4.36 (d, J=6.0 Hz, 2H), 1.46 (d, J=3.8 Hz, 9H), 1.43 (t, J=7.1 Hz, 3H).

### Methyl (2-(4-(((tert-butoxycarbonyl)amino)methyl)phenyl)thiazole-4-carbonyl)-L-serinate (35.4)

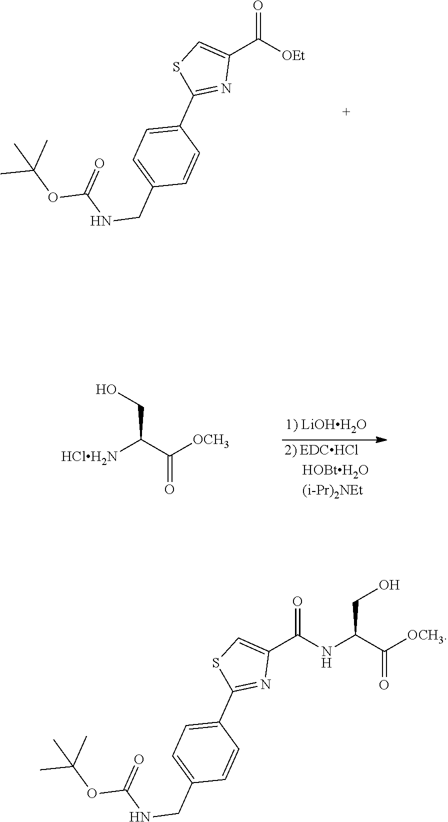

Ethyl 2-(4-(((tert-butoxycarbonyl)amino)methyl)phenyl)thiazole-4-carboxylate (5.003 g, 13.8 mmole) was dissolved in 4/1/1 THF/methanol/water (28 mL). Lithium hydroxide monohydrate (1.160 g, 27.6 mmole) was added and the mixture was stirred at RT for 3 hours. Water (50 mL) was added and the solution was treated with 1N aq. HCl to pH=4. The mixture was extracted with ethyl acetate (3×15 mL) and the combined organics were dried over magnesium sulfate, filtered, and concentrated. The residue was suspended in DCM (28 mL) and L-serine methyl ester hydrochloride (2.589 g, 16.6 mmole) was added. N,N-Diisopropylethylamine (4.80 mL, 27.6 mmole), HOBt·H₂O (2.554 g, 16.7 mmole), and EDC·HCl (3.187 g, 16.6 mmole) were added and the mixture was stirred at RT for 18 hours. Water (50 mL) was added and the two layers were separated. The aqueous layer was extracted with DCM (2×20 mL) and the combined organics were dried over magnesium sulfate, filtered, and concentrated. The crude residue was purified by silica gel chromatography to provide methyl (2-(4-(((tert-butoxycarbonyl)amino)methyl)phenyl)thiazole-4-carbonyl)-L-serinate as a pale yellow solid (3.793 g, 63%). ¹H NMR (400 MHz, CDCl₃): δ 8.25 (d, J=7.5 Hz, 1H), 8.09 (s, 1H), 7.93-7.85 (m, 2H), 7.35 (d, J=7.9 Hz, 2H), 5.00 (s, 1H), 4.87 (dt, J=7.5, 3.8 Hz, 1H), 4.36 (d, J=6.0 Hz, 2H), 4.17-4.04 (m, 2H), 3.84 (s, 3H), 2.95 (t, J=6.1 Hz, 1H), 1.48 (s, 9H).

### Methyl O-(tert-butyldimethylsilyl)-N-(2-(4-((2-(2-methoxyethoxy)acetamido)methyl)phenyl)thiazole-4-carbonyl)-L-serinate (35.5)

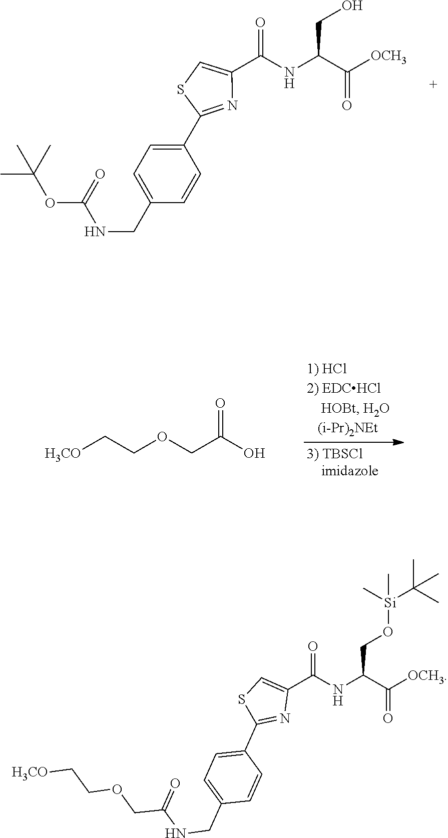 [SMILES: COC(=O)[C@H](CO)NC(=O)c1csc(-c2ccc(CNC(=O)OC(C)(C)C)cc2)n1 ; COCCOCC(=O)O ; COCCOCC(=O)NCc1ccc(-c2nc(C(=O)N[C@@H](CO[Si](C)(C)C(C)(C)C)C(=O)OC)cs2)cc1]

Methyl (2-(4-(((tert-butoxycarbonyl)amino)methyl)phenyl)thiazole-4-carbonyl)-L-serinate (0.700 g, 1.61 mmole) was dissolved in DCM (3.2 mL). HCl (4M solution in 1,4-dioxane, 1.60 mL, 6.40 mmole) was added and the mixture was stirred at RT for 2 hours, then was concentrated. The residue was suspended in DCM (3.2 mL) and 2-(2-methoxyethoxy)acetic acid (0.220 mL, 1.94 mmole) was added. N,N-Diisopropylethylamine (0.560 mL, 3.22 mmole), HOBt·H₂O (0.297 g, 1.94 mmole), and EDC·HCl (0.377 g, 1.97 mmole) were added and the mixture was stirred at RT for 3 days. Water (25 mL) was added and the mixture was extracted with DCM (3×10 mL). The combined organics were dried over magnesium sulfate, filtered, and concentrated. The residue was dissolved in DCM (3.2 mL) and imidazole (0.129 g, 1.89 mmole) and tert-butyldimethylchlorosilane (0.273, 1.81 mmole) were added. The mixture was stirred at RT for 18 hours and water (25 mL) was added. The mixture was extracted with DCM (3×10 mL). The combined organics were dried over magnesium sulfate, filtered, and concentrated. The crude residue was purified by silica gel chromatography to provide methyl O-(tert-butyldimethylsilyl)-N-(2-(4-((2-(2-methoxyethoxy)acetamido)methyl)phenyl)thiazole-4-carbonyl)-L-serinate as a colorless oil (0.838 g, 92%). ¹H NMR (400 MHz, CDCl₃): δ 8.22 (d, J=8.7 Hz, 1H), 8.10 (s, 1H), 7.98-7.89 (m, 2H), 7.48 (s, 1H), 7.43-7.35 (m, 2H), 4.86 (dt, J=8.7, 3.0 Hz, 1H), 4.54 (d, J=5.9 Hz, 2H), 4.21 (dd, J=10.1, 2.6 Hz, 1H), 4.08 (s, 2H), 3.95 (dd, J=10.1, 3.4 Hz, 1H), 3.79 (s, 3H), 3.74-3.66 (m, 2H), 3.56-3.49 (m, 2H), 3.28 (s, 3H), 0.92 (s, 9H), 0.08 (s, 3H), 0.06 (s, 3H).

### Methyl O-acetyl-N—(O-(tert-butyldimethylsilyl)-N-(2-(4-((2-(2-methoxyethoxy)acetamido)methyl)phenyl)thiazole-4-carbonyl)-L-seryl)-L-serinate (35.6)

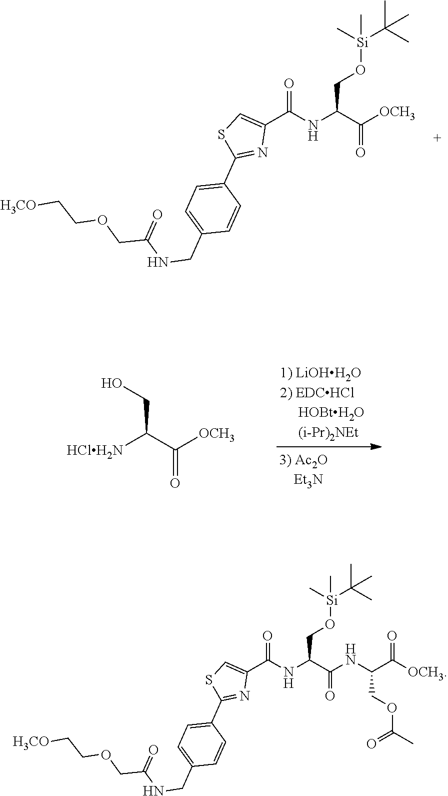

Methyl O-(tert-butyldimethylsilyl)-N-(2-(4-((2-(2-methoxyethoxy)acetamido)methyl)phenyl)thiazole-4-carbonyl)-L-serinate (0.838 g, 1.48 mmole) was dissolved in 4/1/1 THF/methanol/water (3 mL). Lithium hydroxide monohydrate (0.125 g, 2.98 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered, and concentrated. The residue was dissolved in DCM (3 mL) and L-serine methyl ester hydrochloride (0.282 g, 1.81 mmole) was added. N,N-Diisopropylethylamine (0.520 mL, 2.99 mmole), HOBt·H₂O (0.277 g, 1.81 mmole), and EDC·HCl (0.343 g, 1.79 mmole) were added and the mixture was stirred at RT for 18 hours. Water (25 mL) was added and the mixture was extracted with DCM (3×10 mL). The combined organics were dried over magnesium sulfate, filtered, and concentrated. The residue was dissolved in DCM (3 mL) and triethylamine (0.230 mL, 1.64 mmole) and acetic anhydride (0.155 mL, 1.64 mmole) were added. The mixture was stirred at RT for 5 hours and water (25 mL) was added. The mixture was extracted with DCM (3×10 mL) and the combined organics were dried over magnesium sulfate, filtered, and concentrated. The crude residue was purified by silica gel chromatography to provide methyl O-acetyl-N—(O-(tert-butyldimethylsilyl)-N-(2-(4-((2-(2-methoxyethoxy)acetamido)methyl)phenyl)thiazole-4-carbonyl)-L-seryl)-L-serinate as a colorless oil (0.593 g, 58%). ¹H NMR (400 MHz, CDCl₃): δ 8.29-8.22 (m, 1H), 8.11 (d, J=2.4 Hz, 1H), 7.95 (dt, J=8.5, 2.0 Hz, 2H), 7.48 (t, J=6.4 Hz, 2H), 7.44-7.36 (m, 2H), 4.90 (tt, J=7.4, 3.8 Hz, 1H), 4.71-4.63 (m, 1H), 4.54 (d, J=6.0 Hz, 2H), 4.49 (ddd, J=11.0, 7.1, 3.9 Hz, 1H), 4.34 (dd, J=11.4, 3.6 Hz, 1H), 4.23 (dt, J=9.9, 3.4 Hz, 1H), 4.09 (d, J=4.9 Hz, 2H), 3.85-3.80 (m, 1H), 3.77 (d, J=4.2 Hz, 3H), 3.73-3.66 (m, 2H), 3.57-3.49 (m, 2H), 3.28 (s, 3H), 2.01 (s, 3H), 0.96 (s, 9H), 0.19-0.12 (m, 6H).

### Methyl 2-(2-(2-(4-((2-(2-methoxyethoxy)acetamido)methyl)phenyl)thiazole-4-carboxamido)acrylamido)acrylate (35)

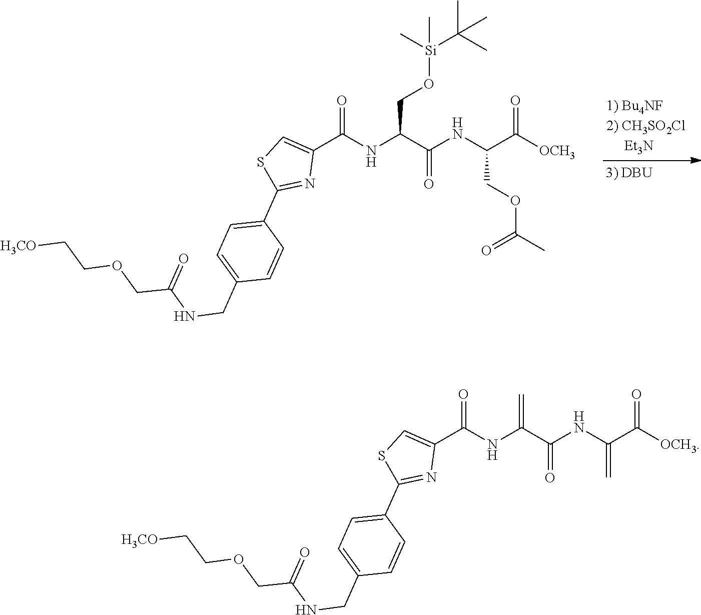 [SMILES: COCCOCC(=O)NCc1ccc(-c2nc(C(=O)N[C@@H](CO[Si](C)(C)C(C)(C)C)C(=O)N[C@@H](COC(C)=O)C(=O)OC)cs2)cc1 ; C=C(NC(=O)c1csc(-c2ccc(CNC(=O)COCCOC)cc2)n1)C(=O)NC(=C)C(=O)OC]

Methyl O-acetyl-N-(0-(tert-butyldimethylsilyl)-N-(2-(4-((2-(2-methoxyethoxy)acetamido)methyl)phenyl)thiazole-4-carbonyl)-L-seryl)-L-serinate (0.593 g, 0.853 mmole) was dissolved in THE (3.2 mL) and TBAF (1M solution in THF, 2.10 mL, 2.10 mmole) was added. The solution was stirred at RT for 90 minutes and water (25 mL) was added. The mixture was extracted with ethyl acetate (3×10 mL) and the combined organics were dried over magnesium sulfate, filtered, and concentrated. The residue was dissolved in DCM (3.2 mL) and cooled to 0° C. Triethylamine (0.180 mL, 1.28 mmole) and methanesulfonyl chloride (0.099 mL, 1.28 mmole) were added and the mixture was stirred at 0° for 60 minutes. Water (25 mL) was added and the mixture was extracted with DCM (3×10 mL). The combined organics were dried over magnesium sulfate, filtered, and concentrated. The residue was dissolved in THE (3.2 mL) and cooled to 0° C. DBU (0.180 mL, 1.20 mmole) was added and the mixture was stirred at 0° for 90 minutes. Water (25 mL) was added and the mixture was extracted with ethyl acetate (3×10 mL). The combined organics were dried over magnesium sulfate, filtered, and concentrated. The crude residue was purified by silica gel chromatography to provide methyl 2-(2-(2-(4-((2-(2-methoxyethoxy)acetamido)methyl)phenyl)thiazole-4-carboxamido)acrylamido)acrylate as a white solid (0.180 g, 42%). ¹H NMR (400 MHz, CDCl₃): δ 10.02 (s, 1H), 8.55 (s, 1H), 8.15 (s, 1H), 8.04-7.94 (m, 2H), 7.48 (s, 1H), 7.44-7.38 (m, 2H), 6.79 (d, J=2.2 Hz, 1H), 6.71 (s, 1H), 6.04 (d, J=1.3 Hz, 1H), 5.51 (t, J=1.9 Hz, 1H), 4.54 (d, J=6.0 Hz, 2H), 4.08 (s, 2H), 3.91 (s, 3H), 3.74-3.67 (m, 2H), 3.59-3.50 (m, 2H), 3.28 (s, 3H).

### Compound 36: Methyl 2-(2-(2-(4-((tetrahydro-2H-pyran-4-carboxamido)methyl)phenyl)thiazole-4-carboxamido)acrylamido)acrylate

### Methyl O-(tert-butyldimethylsilyl)-N-(2-(4-((tetrahydro-2H-pyran-4-carboxamido)methyl)phenyl)thiazole-4-carbonyl)-L-serinate (36.1)

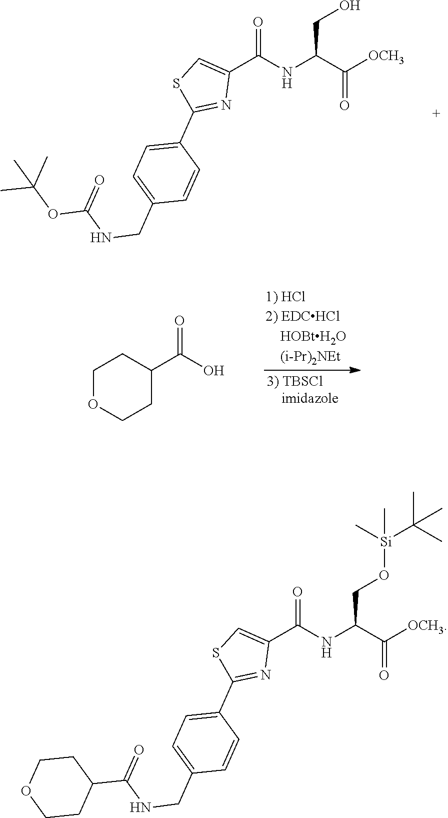 [SMILES: COC(=O)[C@H](CO)NC(=O)c1csc(-c2ccc(CNC(=O)OC(C)(C)C)cc2)n1 ; O=C(O)C1CCOCC1 ; COC(=O)[C@H](CO[Si](C)(C)C(C)(C)C)NC(=O)c1csc(-c2ccc(CNC(=O)C3CCOCC3)cc2)n1]

Methyl (2-(4-(((tert-butoxycarbonyl)amino)methyl)phenyl)thiazole-4-carbonyl)-L-serinate (Example 35.4, 0.603 g, 1.38 mmole) was dissolved in DCM (2.8 mL). HCl (4M solution in 1,4-dioxane) was added and the mixture was stirred at RT for 2 hours, then was concentrated. The residue was suspended in DCM (2.8 mL) and tetrahydro-2H-pyran-4-carboxylic acid (0.218 g, 1.40 mmole) was added. N,N-Diisopropylethylamine (0.480 mL, 2.76 mmole), HOBt·H₂O (0.256 g, 1.67 mmole), and EDC·HCl (0.324 g, 1.69 mmole) were added and the mixture was stirred at RT for 18 hours. Water (25 mL) was added and the mixture was extracted with DCM (3×10 mL). The combined organics were dried over magnesium sulfate, filtered, and concentrated. The residue was dissolved in DCM (5.6 mL) and imidazole (0.112 g, 1.65 mmole) and tert-butyldimethylchlorosilane (0.237 g, 1.57 mmole) were added. The mixture was stirred at RT for 5 hours and water (25 mL) was added. The mixture was extracted with DCM (3×10 mL) and the combined organics were dried over magnesium sulfate, filtered, and concentrated. The crude residue was purified by silica gel chromatography to provide methyl O-(tert-butyldimethylsilyl)-N-(2-(4-((tetrahydro-2H-pyran-4-carboxamido)methyl)phenyl)thiazole-4-carbonyl)-L-serinate as a pale yellow solid (0.535 g, 69%). ¹H NMR (400 MHz, CDCl₃): δ 8.22 (d, J=8.6 Hz, 1H), 8.08 (s, 1H), 7.96-7.88 (m, 2H), 7.38-7.31 (m, 2H), 5.94 (s, 1H), 4.89-4.80 (m, 1H), 4.51 (d, J=5.8 Hz, 2H), 4.21 (dd, J=10.1, 2.6 Hz, 1H), 4.03 (ddd, J=11.5, 4.0, 2.2 Hz, 2H), 3.95 (dd, J=10.1, 3.4 Hz, 1H), 3.79 (s, 3H), 3.43 (td, J=11.4, 2.9 Hz, 2H), 2.42 (tt, J=11.1, 4.5 Hz, 1H), 1.94-1.74 (m, 4H), 0.92 (s, 9H), 0.07 (s, 3H), 0.06 (s, 3H).

Methyl O-acetyl-N—(O-(tert-butyldimethylsilyl)-N-(2-(4-((tetrahydro-2H-pyran-4-carboxamido)methyl)phenyl)thiazole-4-carbonyl)-L-seryl)-L-serinate (36.2)

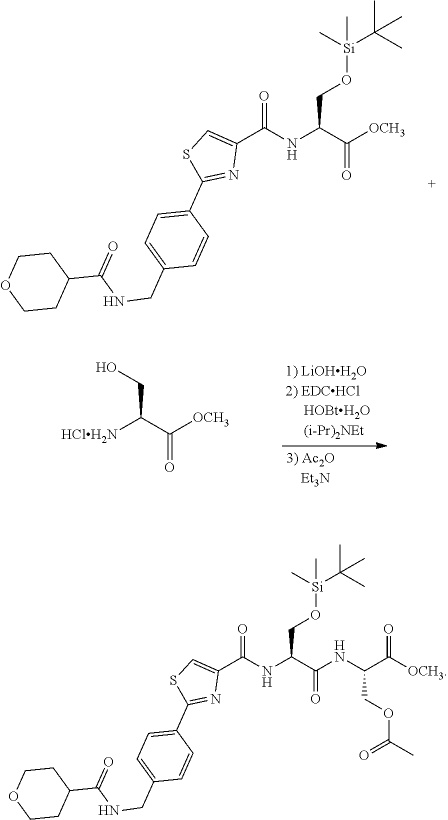

Methyl O-(tert-butyldimethylsilyl)-N-(2-(4-((tetrahydro-2H-pyran-4-carboxamido)methyl)phenyl)thiazole-4-carbonyl)-L-serinate (0.535 g, 0.952 mmole) was dissolved in 4/1/1 THF/methanol/water (2 mL). Lithium hydroxide monohydrate (0.063 g, 1.60 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered, and concentrated. The residue was dissolved in DCM (4 mL) and L-serine methyl ester hydrochloride (0.182 g, 1.17 mmole) was added. N,N-Diisopropylethylamine (0.340 mL, 1.95 mmole), HOBt·H₂O (0.175 g, 1.14 mmole), and EDC·HCl (0.223 g, 1.16 mmole) were added and the mixture was stirred at RT for 3 days. Water (25 mL) was added and the mixture was extracted with DCM (3×10 mL). The combined organics were dried over magnesium sulfate, filtered, and concentrated. The residue was dissolved in DCM (4 mL) and triethylamine (0.150 mL, 1.07 mmole) and acetic anhydride (0.099 mL, 1.09 mmole) were added. The mixture was stirred at RT for 3 hours and water (25 mL) was added. The mixture was extracted with DCM (3×10 mL) and the combined organics were dried over magnesium sulfate, filtered, and concentrated. The crude residue was purified by silica gel chromatography to provide methyl O-acetyl-N-(0-(tert-butyldimethylsilyl)-N-(2-(4-((tetrahydro-2H-pyran-4-carboxamido)methyl)phenyl)thiazole-4-carbonyl)-L-seryl)-L-serinate as a colorless oil (0.441 g, 88%). ¹H NMR (400 MHz, CDCl₃): δ 8.26 (d, J=7.0 Hz, 1H), 8.10 (d, J=2.4 Hz, 1H), 7.97-7.90 (m, 2H), 7.48 (d, J=7.8 Hz, 1H), 7.38-7.30 (m, 2H), 5.90 (t, J=5.9 Hz, 1H), 4.90 (dq, J =7.0, 3.5 Hz, 1H), 4.66 (td, J=7.3, 3.7 Hz, 1H), 4.51 (d, J=5.8 Hz, 2H), 4.47 (dd, J=6.7, 4.0 Hz, 1H), 4.34 (dd, J=11.4, 3.6 Hz, 1H), 4.23 (dd, J=9.8, 3.7 Hz, 1H), 4.04 (ddd, J=11.5, 4.2, 2.3 Hz, 2H), 3.84-3.79 (m, 1H), 3.77 (s, 3H), 3.43 (td, J=11.5, 2.9 Hz, 2H), 2.42 (tt, J=11.0, 4.4 Hz, 1H), 2.01 (s, 3H), 1.92-1.75 (m, 4H), 0.96 (s, 9H), 0.17 (s, 3H), 0.16 (s, 3H).

### Methyl 2-(2-(2-(4-((tetrahydro-2H-pyran-4-carboxamido)methyl)phenyl)thiazole-4-carboxamido)acrylamido)acrylate (36)

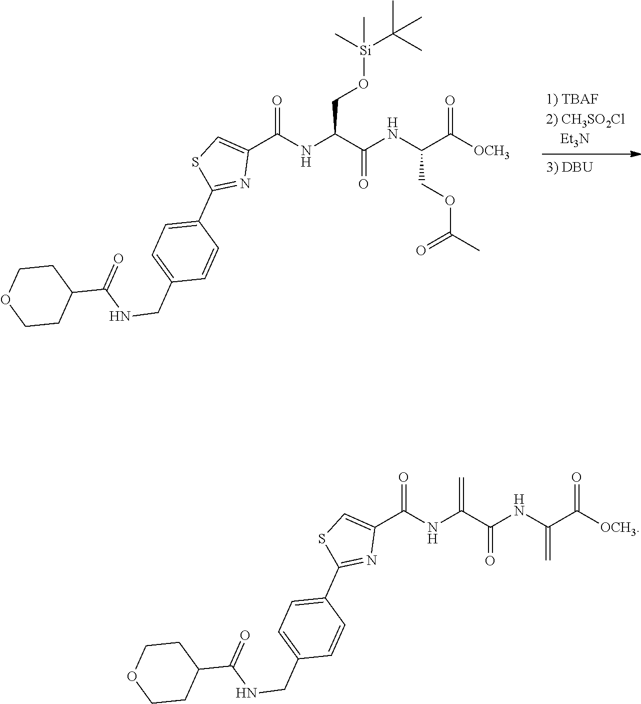 [SMILES: COC(=O)[C@H](COC(C)=O)NC(=O)[C@H](CO[Si](C)(C)C(C)(C)C)NC(=O)c1csc(-c2ccc(CNC(=O)C3CCOCC3)cc2)n1 ; C=C(NC(=O)c1csc(-c2ccc(CNC(=O)C3CCOCC3)cc2)n1)C(=O)NC(=C)C(=O)OC]

Methyl O-acetyl-N-(0-(tert-butyldimethylsilyl)-N-(2-(4-((tetrahydro-2H-pyran-4-carboxamido)methyl)phenyl)thiazole-4-carbonyl)-L-seryl)-L-serinate (0.441 g, 0.638 mmole) was dissolved in THF (1.3 mL) and TBAF (1M solution in THF, 1.60 mL, 1.60 mmole) was added. The mixture was stirred at RT for 2 hours and water (25 mL) was added. The mixture was extracted with ethyl acetate (3×10 mL) and the combined organics were dried over magnesium sulfate, filtered, and concentrated. The residue was dissolved in DCM (2.6 mL) and cooled to 0° C. Triethylamine (0.135 mL, 0.963 mmole) and methanesulfonyl chloride (0.075 mL, 0.969 mmole) were added and the mixture was stirred at 0° for 60 minutes. Water (25 mL) was added and the mixture was extracted with DCM (3×10 mL). The combined organics were dried over magnesium sulfate, filtered, and concentrated. The residue was dissolved in THF (2.6 mL) and cooled to 0° C. DBU (0.150 mL, 1.00 mmole) was added and the mixture was stirred at 0° for 45 minutes. Water (25 mL) was added and the mixture was extracted with ethyl acetate (3×10 mL). The combined organics were filtered, then dried over magnesium sulfate, filtered, and concentrated. The residue was suspended in a small amount of ethyl acetate and the solid was collected by filtration. The two crops were combined to provide methyl 2-(2-(2-(4-((tetrahydro-2H-pyran-4-carboxamido)methyl)phenyl)thiazole-4-carboxamido)acrylamido)acrylate as a white solid (0.101 g, 32%). ¹H NM/R (400 MHz, CDCl₃): δ 10.01 (s, 1H), 8.56 (s, 1H), 8.14 (s, 1H), 8.02-7.92 (m, 2H), 7.36 (d, J=8.0 Hz, 2H), 6.78 (d, J=2.2 Hz, 1H), 6.70 (s, 1H), 6.04 (d, J 1.3 Hz, 1H), 5.85 (s, 1H), 5.51 (t, J 1.9 Hz, 1H), 4.52 (d, J 5.8 Hz, 2H), 4.04 (ddd, J 11.6, 4.3, 2.4 Hz, 2H), 3.91 (s, 3H), 3.43 (td,J=11.4, 3.0 Hz, 2H), 2.41 (tt,J=11.0, 4.6 Hz, 1H), 1.93-1.74 (in, 4H).

### Compound 37: Methyl 2-(2-(2-(4-((6-chlorohexanamido)methyl)phenyl)thiazole-4-carboxamido)acrylamido)acrylate

### Methyl O-(tert-butyldimethylsilyl)-N-(2-(4-((6-chlorohexanamido)methyl)phenyl)thiazole-4-carbonyl)-L-serinate (37.1)

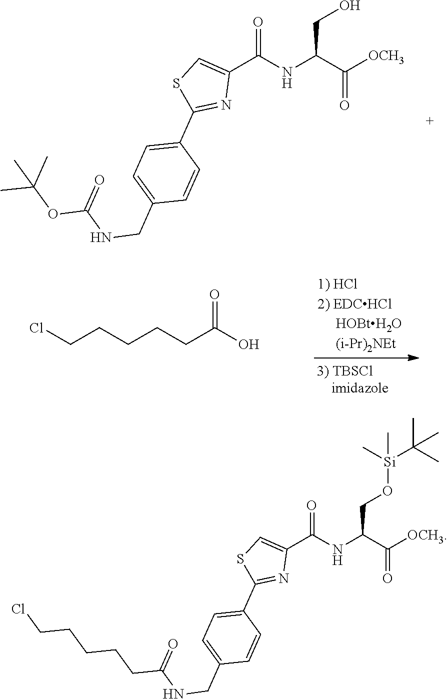 [SMILES: COC(=O)[C@H](CO)NC(=O)c1csc(-c2ccc(CNC(=O)OC(C)(C)C)cc2)n1 ; O=C(O)CCCCCCl ; COC(=O)[C@H](CO[Si](C)(C)C(C)(C)C)NC(=O)c1csc(-c2ccc(CNC(=O)CCCCCCl)cc2)n1]

Methyl (2-(4-(((tert-butoxycarbonyl)amino)methyl)phenyl)thiazole-4-carbonyl)-L-serinate (Example 35.4, 0.603 g, 1.38 mmole) was dissolved in DCM (2.8 mL). HCl (4M solution in 1,4-dioxane, 1.40 mL, 5.60 mmole) was added and the mixture was stirred at RT for 3 hours, then was concentrated. The residue was suspended in DCM (2.8 mL) and a solution of 6-chlorohexanoic acid (0.249 g, 1.65 mmole) in DCM (0.5 mL) was added. N,N-Diisopropylethylamine (0.480 mL, 2.76 mmole), HOBt·H₂O (0.254 g, 1.66 mmole), and EDC·HCl (0.320 g, 1.67 mmole) were added and the mixture was stirred at RT for 3 days. Water (25 mL) was added and the mixture was extracted with DCM (3×10 mL). The combined organics were dried over magnesium sulfate, filtered, and concentrated. The residue was dissolved in DCM (2.8 mL) and imidazole (0.114 g, 1.67 mmole) and tert-butyldimethylchlorosilane (0.231 g, 1.53 mmole) were added. The mixture was stirred at RT for 3 hours and water (25 mL) was added. The mixture was extracted with DCM (3×10 mL) and the combined organics were dried over magnesium sulfate, filtered, and concentrated. The crude residue was purified by silica gel chromatography to provide methyl O-(tert-butyldimethylsilyl)-N-(2-(4-((6-chlorohexanamido)methyl)phenyl)thiazole-4-carbonyl)-L-serinate as a white solid (0.612 g, 76%). ¹H NMR (400 MHz, CDCl₃): δ 8.21 (d, J=8.6 Hz, 1H), 8.07 (s, 1H), 7.95-7.89 (m, 2H), 7.39-7.31 (m, 2H), 5.93 (t, J=6.0 Hz, 1H), 4.89-4.81 (m, 1H), 4.50 (d, J=5.8 Hz, 2H), 4.21 (dd, J=10.1, 2.7 Hz, 1H), 3.95 (dd, J=10.1, 3.4 Hz, 1H), 3.79 (s, 3H), 3.54 (t, J=6.6 Hz, 2H), 2.28 (t, J=7.5 Hz, 2H), 1.80 (dt, J=8.1, 6.7 Hz, 2H), 1.73 (tt, J=8.3, 6.4 Hz, 2H), 1.56-1.47 (m, 2H), 0.92 (s, 9H), 0.08 (s, 3H), 0.06 (s, 3H).

### Methyl O-acetyl-N—(O-(tert-butyldimethylsilyl)-N-(2-(4-((6-chlorohexanamido)methyl)phenyl)thiazole-4-carbonyl)-L-seryl)-L-serinate (37.2)

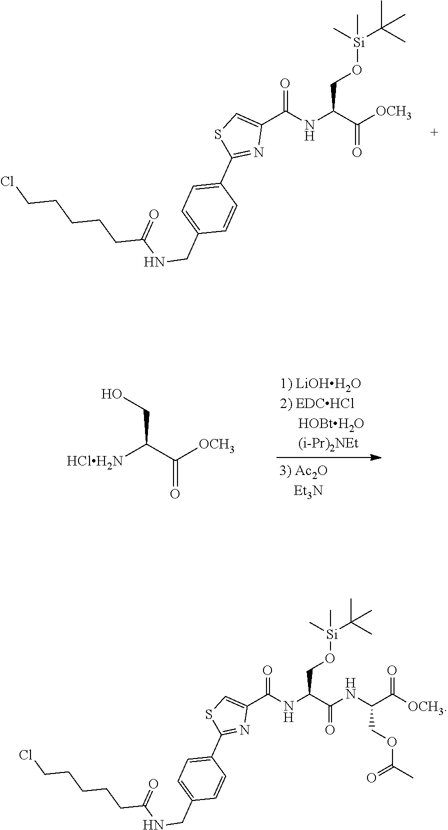

Methyl O-(tert-butyldimethylsilyl)-N-(2-(4-((6-chlorohexanamido)methyl)phenyl)thiazole-4-carbonyl)-L-serinate (0.612 g, 1.05 mmole) was dissolved in 4/1/1 THF/methanol/water (2 mL). Lithium hydroxide monohydrate (0.065 g, 1.55 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered, and concentrated. The residue was dissolved in DCM (2 mL) and L-serine methyl ester hydrochloride (0.192 g, 1.23 mmole) was added. N,N-Diisopropylethylamine (0.370 mL, 2.12. mmole), HOBt·H₂O (0.199 g, 1.30 mmole), and EDC·HCl (0.240 g, 1.25 mmole) were added and the mixture was stirred at RT for 18 hours. Water (25 mL) was added and the mixture was extracted with DCM (3×10 mL). The combined organics were dried over magnesium sulfate, filtered, and concentrated. The residue was dissolved in DCM (4 mL) and triethylamine (0.165 mL, 1.18 mmole) and acetic anhydride (0.110 mL, 1.16 mmole) were added. The mixture was stirred at RT for 3 hours and water (25 mL) was added. The mixture was extracted with DCM (3×10 mL) and the combined organics were dried over magnesium sulfate, filtered, and concentrated. The crude residue was purified by silica gel chromatography to provide methyl O-acetyl-N—(O-(tert-butyldimethylsilyl)-N-(2-(4-((6-chlorohexanamido)methyl)phenyl)thiazole-4-carbonyl)-L-seryl)-L-serinate as a colorless oil (0.288 g, 39%). ¹H NMR (400 MHz, CDCl₃): δ 8.26 (d, J=7.2 Hz, 1H), 8.10 (d, J=2.3 Hz, 1H), 7.98-7.88 (m, 2H), 7.47 (d, J=7.8 Hz, 1H), 7.39-7.31 (m, 2H), 5.84 (t, J=5.6 Hz, 1H), 4.90 (dt, J=7.5, 3.7 Hz, 1H), 4.66 (td, J=7.3, 3.7 Hz, 1H), 4.54-4.47 (m, 3H), 4.34 (dd, J=11.4, 3.6 Hz, 1H), 4.22 (dt, J=9.8, 3.4 Hz, 1H), 3.84-3.79 (m, 1H), 3.77 (d, J=4.1 Hz, 3H), 3.55 (t, J=6.6 Hz, 2H), 2.27 (t, J=7.5 Hz, 2H), 2.05 (s, 3H), 1.86-1.77 (m, 2H), 1.77-1.66 (m, 2H), 1.56-1.48 (m, 2H), 0.96 (s, 9H), 0.16 (t, J=4.8 Hz, 6H).

### Methyl 2-(2-(2-(4-((6-chlorohexanamido)methyl)phenyl)thiazole-4-carboxamido)acrylamido)acrylate (37)

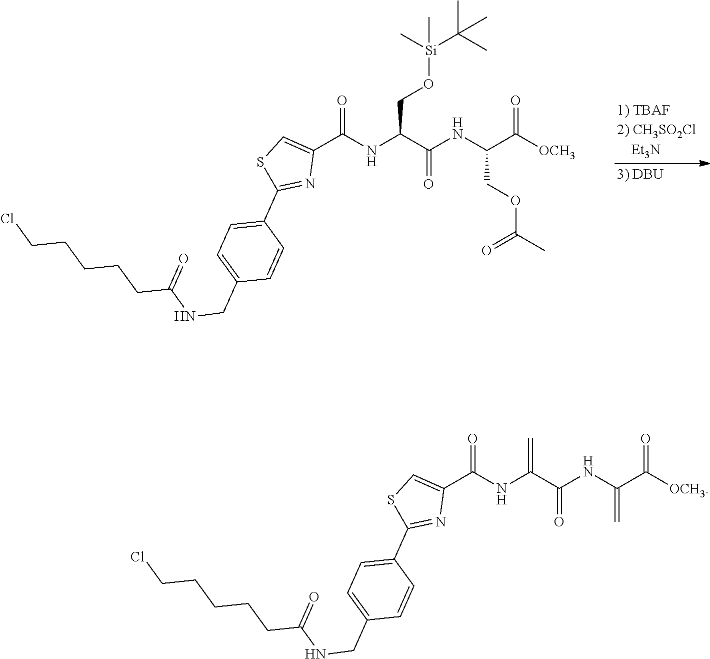 [SMILES: COC(=O)[C@H](COC(C)=O)NC(=O)[C@H](CO[Si](C)(C)C(C)(C)C)NC(=O)c1csc(-c2ccc(CNC(=O)CCCCCCl)cc2)n1 ; C=C(NC(=O)c1csc(-c2ccc(CNC(=O)CCCCCCl)cc2)n1)C(=O)NC(=C)C(=O)OC]

Methyl O-acetyl-N—(O-(tert-butyldimethylsilyl)-N-(2-(4-((6-chlorohexanamido)methyl)phenyl)thiazole-4-carbonyl)-L-seryl)-L-serinate (0.288 g, 0.405 mmole) was dissolved in THE (1.6 mL). TBAF (1M solution in THF, 1.00 mL, 1.00 mmole) was added and the solution was stirred at RT for 2 hours. Water (25 mL) was added and the mixture was extracted with ethyl acetate (3×10 mL). The combined organics were dried over magnesium sulfate, filtered, and concentrated. The residue was dissolved in DCM (1.6 mL) and cooled to 0° C. Triethylamine (0.086 mL, 0.614 mmole) and methanesulfonyl chloride (0.048 mL, 0.620 mmole) were added and the mixture was stirred at 0° for 45 minutes. Water (25 mL) was added and the mixture was extracted with DCM (3×10 mL). The combined organics were dried over magnesium sulfate, filtered, and concentrated. The residue was dissolved in THE (1.6 mL) and cooled to 0° C. DBU (0.091 mL, 0.608 mmole) was added and the mixture was stirred at 0° for 30 minutes. Water (25 mL) was added and the mixture was extracted with ethyl acetate (3×10 mL). The combined organics were dried over magnesium sulfate, filtered, and concentrated. The crude residue was purified by silica gel chromatography to provide methyl 2-(2-(2-(4-((6-chlorohexanamido)methyl)phenyl)thiazole-4-carboxamido)acrylamido)acrylate as a white solid (0.100 g, 48%). ¹H NMR (400 MHz, CDCl₃): δ 10.03 (s, 1H), 8.58 (s, 1H), 8.15 (s, 1H), 8.04-7.94 (m, 2H), 7.44-7.34 (m, 2H), 6.80 (d, J=2.3 Hz, 1H), 6.73 (s, 1H), 6.06 (d, J=1.3 Hz, 1H), 5.86 (s, 1H), 5.53 (t, J=1.9 Hz, 1H), 4.53 (d, J=5.8 Hz, 2H), 3.93 (s, 3H), 3.57 (t, J=6.6 Hz, 2H), 2.30 (t, J=7.5 Hz, 2H), 1.90-1.79 (m, 2H), 1.75 (tt, J=8.3, 6.4 Hz, 2H), 1.58-1.49 (m, 2H).

### Compound 38: Methyl 2-(2-(2-(4-((3-((tert-butoxycarbonyl)amino)propanamido)methyl) phenyl)thiazole-4-carboxamido)acrylamido)acrylate

### Methyl N-(2-(4-((3-((tert-butoxycarbonyl)amino)propanamido)methyl)phenyl)thiazole-4-carbonyl)-O-(tert-butyldimethylsilyl)-L-serinate (38.1)

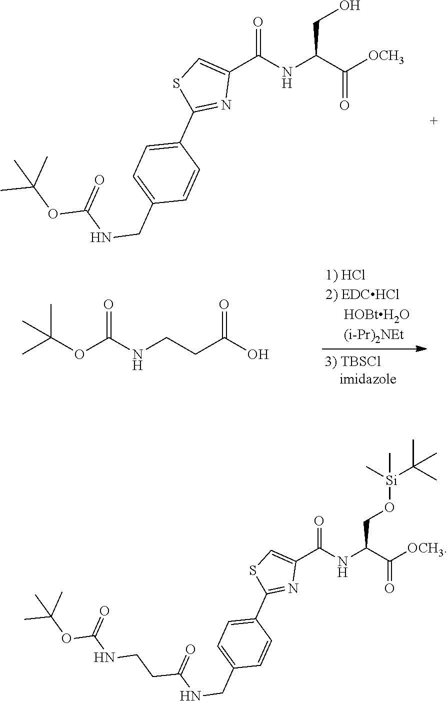 [SMILES: COC(=O)[C@H](CO)NC(=O)c1csc(-c2ccc(CNC(=O)OC(C)(C)C)cc2)n1 ; CC(C)(C)OC(=O)NCCC(=O)O ; COC(=O)[C@H](CO[Si](C)(C)C(C)(C)C)NC(=O)c1csc(-c2ccc(CNC(=O)CCNC(=O)OC(C)(C)C)cc2)n1]

Methyl (2-(4-(((tert-butoxycarbonyl)amino)methyl)phenyl)thiazole-4-carbonyl)-L-serinate (Example 35.4, 0.612 g, 1.41 mmole) was dissolved in DCM (5.6 mL). HCl (4M solution in 1,4-dioxane, 1.40 mL, 5.60 mmole) was added and the mixture was stirred at RT for 3 hours, then was concentrated. The residue was suspended in DCM (5.6 mL) and (tert-butoxycarbonyl)-β-alanine (0.317 g, 1.68 mmole) was added. N,N-Diisopropylethylamine (0.480 mL, 2.76 mmole), HOBt·H₂O (0.260 g, 1.70 mmole), and EDC·HCl (0.320 g, 1.67 mmole) were added and the mixture was stirred at RT for 3 days. Water (25 mL) was added and the mixture was extracted with DCM (3×10 mL). The combined organics were dried over magnesium sulfate, filtered, and concentrated. The residue was dissolved in DCM (5.6 mL) and imidazole (0.112 g, 1.65 mmole) and tert-butyldimethylchlorosilane (0.231 g, 1.53 mmole) were added. The mixture was stirred at RT for 3 hours and water (25 mL) was added. The mixture was extracted with DCM (3×10 mL) and the combined organics were dried over magnesium sulfate, filtered, and concentrated. The crude residue was purified by silica gel chromatography to provide methyl N-(2-(4-((3-((tert-butoxycarbonyl)amino)propanamido)methyl)phenyl)thiazole-4-carbonyl)-O-(tert-butyldimethylsilyl)-L-serinate as a colorless solid (0.626 g, 72%). ¹H NMR (400 MHz, CDCl₃): δ 8.21 (d, J=8.7 Hz, 1H), 8.09 (s, 1H), 7.97-7.88 (m, 2H), 7.40-7.32 (m, 2H), 6.15 (s, 1H), 5.15 (s, 1H), 4.86 (dt, J=8.7, 3.1 Hz, 1H), 4.50 (d, J=5.8 Hz, 2H), 4.21 (dd, J=10.0, 2.7 Hz, 1H), 3.95 (dd, J=10.1, 3.4 Hz, 1H), 3.79 (s, 3H), 3.45 (q, J=6.2 Hz, 2H), 2.49 (t, J=6.0 Hz, 2H), 1.42 (s, 9H), 0.92 (s, 9H), 0.07 (s, 3H), 0.06 (s, 3H).

### Methyl O-acetyl-N—(N-(2-(4-((3-((tert-butoxycarbonyl)amino)propanamido)methyl)phenyl) thiazole-4-carbonyl)-O-(tert-butyldimethylsilyl)-L-seryl)-L-serinate (38.2)

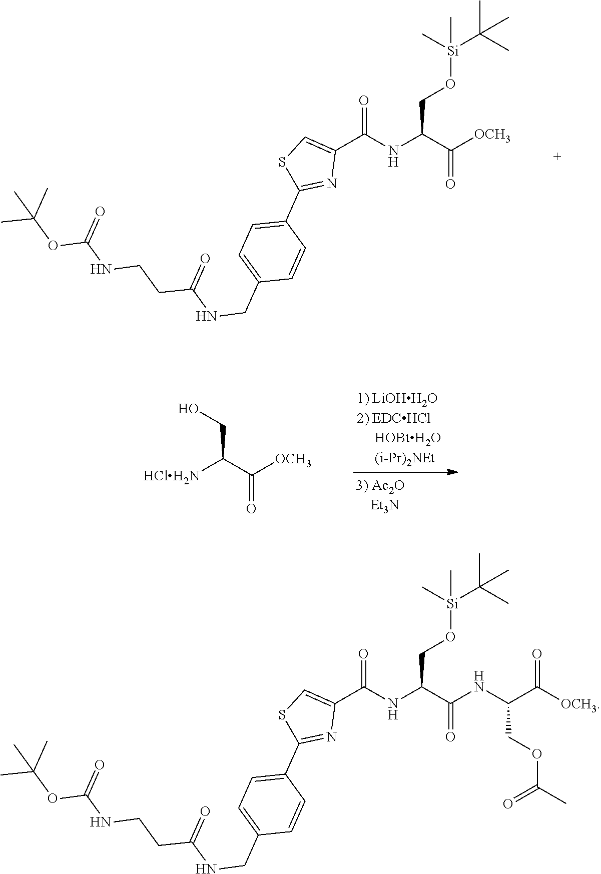

Methyl N-(2-(4-((3-((tert-butoxycarbonyl)amino)propanamido)methyl)phenyl)thiazole-4-carbonyl)-O-(tert-butyldimethylsilyl)-L-serinate (0.626 g, 1.01 mmole) was dissolved in 4/1/1 THF/methanol/water (2 mL). Lithium hydroxide monohydrate (0.066 g, 1.57 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered, and concentrated. The residue was dissolved in DCM (4 mL). L-Serine methyl ester hydrochloride (0.188 g, 1.21 mmole) was added followed by N,N-diisopropylethylamine (0.350 mL, 2.01 mmole), HOBt·H₂O (0.192 g, 1.25 mmole), and EDC·HCl (0.232 g, 1.21 mmole). The mixture was stirred at RT for 18 hours and water (25 mL) was added. The mixture was extracted with DCM (3×10 mL) and the combined organics were dried over magnesium sulfate, filtered, and concentrated. The residue was dissolved in DCM (4 mL) and triethylamine (0.160 mL, 1.14 mmole) and acetic anhydride (0.105 mL, 1.11 mmole) were added. The mixture was stirred at RT for 18 hours and water (25 mL) was added. The mixture was extracted with DCM (3×10 mL) and the combined organics were dried over magnesium sulfate, filtered, and concentrated. The crude residue was purified by silica gel chromatography to provide methyl O-acetyl-N—(N-(2-(4-((3-((tert-butoxycarbonyl)amino)propanamido)methyl)phenyl)thiazole-4-carbonyl)-O-(tert-butyldimethylsilyl)-L-seryl)-L-serinate as a colorless oil (0.315 g, 42%). ¹H NMR (400 MHz, CDCl₃): δ 8.25 (dd, J=7.2, 3.4 Hz, 1H), 8.09 (d, J=4.6 Hz, 1H), 7.97-7.89 (m, 2H), 7.48 (d, J=7.8 Hz, 1H), 7.39-7.31 (m, 2H), 6.26 (s, 1H), 5.18 (s, 1H), 4.89 (tt, J=6.6, 3.8 Hz, 1H), 4.66 (td, J=7.3, 3.8 Hz, 1H), 4.53-4.45 (m, 3H), 4.34 (dd, J=11.4, 3.6 Hz, 1H), 4.22 (dt, J=9.9, 3.8 Hz, 1H), 3.84-3.78 (m, 1H), 3.77 (s, 3H), 3.45 (q, J=6.2 Hz, 2H), 2.49 (t, J=6.0 Hz, 2H), 2.01 (s, 3H), 1.42 (s, 9H), 0.96 (s, 9H), 0.16 (s, 3H), 0.15 (s, 3H).

### Methyl 2-(2-(2-(4-((3-((tert-butoxycarbonyl)amino)propanamido)methyl)phenyl)thiazole-4-carboxamido)acrylamido)acrylate (38)

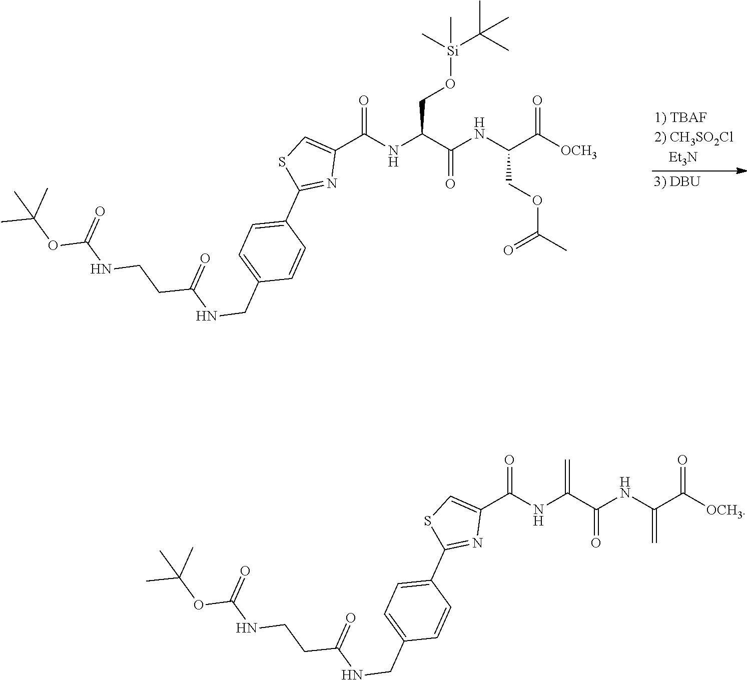 [SMILES: COC(=O)[C@H](COC(C)=O)NC(=O)[C@H](CO[Si](C)(C)C(C)(C)C)NC(=O)c1csc(-c2ccc(CNC(=O)CCNC(=O)OC(C)(C)C)cc2)n1 ; C=C(NC(=O)c1csc(-c2ccc(CNC(=O)CCNC(=O)OC(C)(C)C)cc2)n1)C(=O)NC(=C)C(=O)OC]

Methyl O-acetyl-N—(N-(2-(4-((3-((tert-butoxycarbonyl)amino)propanamido)methyl)phenyl)thiazole-4-carbonyl)-O-(tert-butyldimethylsilyl)-L-seryl)-L-serinate (0.315 g, 0.420 mmole) was dissolved in THE (1.6 mL). TBAF (1M solution in THF, 1.05 mL, 1.05 mmole) was added and the solution was stirred at RT for 2 hours. Water (25 mL) was added and the mixture was extracted with ethyl acetate (3×10 mL). The combined organics were dried over magnesium sulfate, filtered, and concentrated. The residue was dissolved in DCM (1.6 mL) and cooled to 0° C. Triethylamine (0.089 mL, 0.635 mmole) and methanesulfonyl chloride (0.049 mL, 0.633 mmole) were added and the mixture was stirred at 0° for 2 hours, then at RT for 18 hours. Water (25 mL) was added and the mixture was extracted with DCM (3×10 mL). The combined organics were dried over magnesium sulfate, filtered, and concentrated. The residue was dissolved in THE (1.6 mL) and cooled to 0° C. DBU (0.100 mL, 0.669 mmole) was added and the mixture was stirred at 0° for 30 minutes. Water (25 mL) was added and the mixture was extracted with ethyl acetate (3×10 mL). The combined organics were dried over magnesium sulfate, filtered, and concentrated. The crude residue was purified by silica gel chromatography to provide methyl 2-(2-(2-(4-((3-((tert-butoxycarbonyl)amino)propanamido)methyl)phenyl)thiazole-4-carboxamido)acrylamido)acrylate as a white solid (0.096 g, 41%). ¹H NMR (400 MHz, CDCl₃): δ 10.01 (s, 1H), 8.55 (s, 1H), 8.14 (s, 1H), 8.01-7.93 (m, 2H), 7.41-7.33 (m, 2H), 6.78 (d, J=2.2 Hz, 1H), 6.71 (s, 1H), 6.12 (s, 1H), 6.03 (d, J=1.3 Hz, 1H), 5.50 (t, J=1.9 Hz, 1H), 5.15 (s, 1H), 4.50 (d, J=5.9 Hz, 2H), 3.90 (s, 3H), 3.46 (q, J=6.2 Hz, 2H), 2.49 (t, J=6.0 Hz, 2H), 1.43 (s, 9H).

### Compound 39: Methyl 2-(2-(2-(4-((6-azidohexanamido)methyl)phenyl)thiazole-4-carboxamido)acrylamido)acrylate

### Methyl N-(2-(4-((6-bromohexanamido)methyl)phenyl)thiazole-4-carbonyl)-O-(tert-butyldimethylsilyl)-L-serinate (39.1)

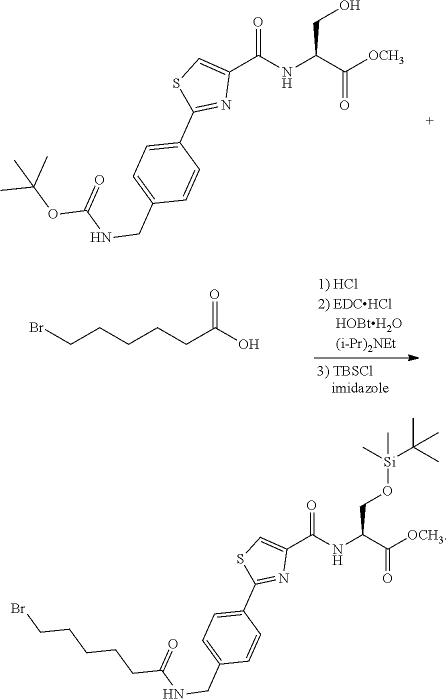 [SMILES: COC(=O)[C@H](CO)NC(=O)c1csc(-c2ccc(CNC(=O)OC(C)(C)C)cc2)n1 ; O=C(O)CCCCCBr ; COC(=O)[C@H](CO[Si](C)(C)C(C)(C)C)NC(=O)c1csc(-c2ccc(CNC(=O)CCCCCBr)cc2)n1]

Methyl (2-(4-(((tert-butoxycarbonyl)amino)methyl)phenyl)thiazole-4-carbonyl)-L-serinate (Example 35.4, 1.202 g, 2.76 mmole) was dissolved in DCM (20 mL). HCl (4M solution in 1,4-dioxane, 2.80 mL, 11.2 mmole) was added and the mixture was stirred at RT for 5 hours, then was concentrated. The residue was suspended in DCM (10 mL) and 6-bromohexanoic acid (0.673 g, 3.45 mmole) was added. N,N-Diisopropylethylamine (0.960 mL, 5.51 mmole), HOBt·H₂O (0.572 g, 3.74 mmole), and EDC·HCl (0.638 g, 3.33 mmole) were added and the mixture was stirred at RT for 18 hours. Water (25 mL) was added and the mixture was extracted with DCM (3×10 mL). The combined organics were dried over magnesium sulfate, filtered, and concentrated. The residue was dissolved in DCM (10 mL) and imidazole (0.214 g, 3.14 mmole) and tert-butyldimethylchlorosilane (0.474 g, 3.14 mmole) were added. The mixture was stirred at RT for 3 hours and water (25 mL) was added. The mixture was extracted with DCM (3×10 mL) and the combined organics were dried over magnesium sulfate, filtered, and concentrated. The crude residue was purified by silica gel chromatography to provide methyl N-(2-(4-((6-bromohexanamido)methyl)phenyl)thiazole-4-carbonyl)-O-(tert-butyldimethylsilyl)-L-serinate as a white solid (0.996 g, 58%). 1H NMR (400 MHz, CDCl₃) δ 8.21 (d, J=8.7 Hz, 1H), 8.05 (s, 1H), 7.94-7.85 (m, 2H), 7.39-7.30 (m, 2H), 6.19-6.01 (m, 1H), 4.84 (dt, J=8.7, 3.1 Hz, 1H), 4.49 (d, J=5.8 Hz, 2H), 4.21 (dd, J=10.1, 2.7 Hz, 1H), 3.95 (dd, J=10.1, 3.4 Hz, 1H), 3.79 (s, 3H), 3.41 (t, J=6.7 Hz, 2H), 2.28 (t, J=7.5 Hz, 2H), 1.88 (dq, J=8.1, 6.8 Hz, 2H), 1.72 (ddd, J=15.3, 7.9, 6.4 Hz, 2H), 1.56-1.45 (m, 2H), 0.92 (s, 9H), 0.08 (s, 3H), 0.06 (s, 3H).

### Methyl N-(2-(4-((6-azidohexanamido)methyl)phenyl)thiazole-4-carbonyl)-O-(tert-butyldimethylsilyl)-L-serinate (39.2)

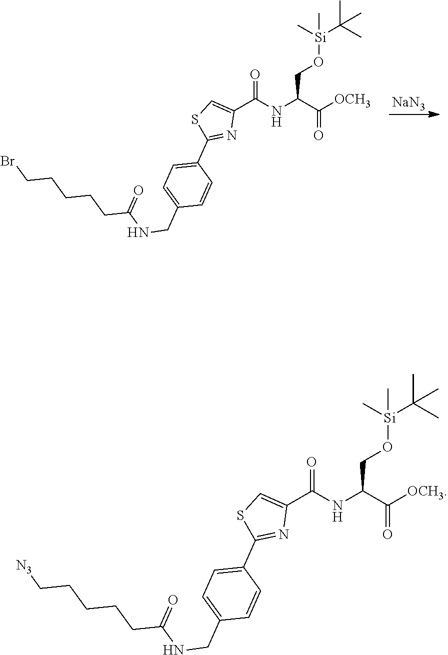 [SMILES: COC(=O)[C@H](CO[Si](C)(C)C(C)(C)C)NC(=O)c1csc(-c2ccc(CNC(=O)CCCCCBr)cc2)n1 ; COC(=O)[C@H](CO[Si](C)(C)C(C)(C)C)NC(=O)c1csc(-c2ccc(CNC(=O)CCCCCN=[N+]=[N-])cc2)n1 ; "NaN3"]

Methyl N-(2-(4-((6-bromohexanamido)methyl)phenyl)thiazole-4-carbonyl)-O-(tert-butyldimethylsilyl)-L-serinate (0.996 g, 1.59 mmole) was dissolved in DMF (3.2 mL). Sodium azide (0.123 g, 1.89 mmole) was added and the mixture was heated to 80° C. for 4 ½hours, then was cooled to RT and diluted with ethyl acetate (25 mL). The mixture was washed with water (3×20 mL) and the organics were dried over magnesium sulfate, filtered, and concentrated. The crude residue was purified by silica gel chromatography to provide methyl N-(2-(4-((6-azidohexanamido)methyl)phenyl)thiazole-4-carbonyl)-O-(tert-butyldimethylsilyl)-L-serinate as a white solid (0.634 g, 68%). ¹H NMR (400 MHz, CDCl₃): δ 8.21 (d, J=8.7 Hz, 1H), 8.08 (s, 1H), 7.97-7.89 (m, 2H), 7.39-7.32 (m, 2H), 5.87 (s, 1H), 4.86 (dt, J=8.7, 2.9 Hz, 1H), 4.50 (d, J=5.8 Hz, 2H), 4.21 (dd, J=10.0, 2.7 Hz, 1H), 3.99-3.91 (m, 1H), 3.79 (s, 3H), 3.28 (t, J=6.8 Hz, 2H), 2.27 (t, J=7.5 Hz, 2H), 1.73 (tt, J=8.2, 6.5 Hz, 2H), 1.68-1.58 (m, 2H), 1.50-1.39 (m, 2H), 0.92 (s, 9H), 0.08 (s, 3H), 0.06 (s, 3H).

### Methyl O-acetyl-N—(N-(2-(4-((6-azidohexanamido)methyl)phenyl)thiazole-4-carbonyl)-O-(tert-butyldimethylsilyl)-L-seryl)-L-serinate (39.3)

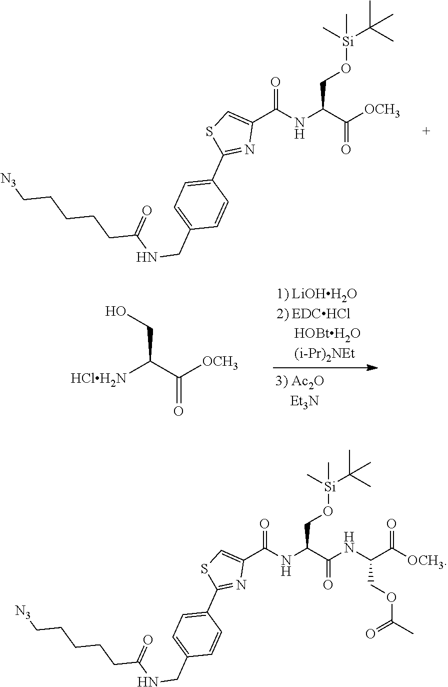

Methyl N-(2-(4-((6-azidohexanamido)methyl)phenyl)thiazole-4-carbonyl)-O-(tert-butyldimethylsilyl)-L-serinate (0.634 g, 1.08 mmole) was dissolved in 4/1/1 THF/methanol/water (2.2 mL). Lithium hydroxide monohydrate (0.070 g, 1.67 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered, and concentrated. The residue was dissolved in DCM (4.4 mL). L-Serine methyl ester hydrochloride (0.202 g, 1.30 mmole) was added followed by N,N-diisopropylethylamine (0.380 mL, 2.18 mmole), HOBt·H₂O (0.199 g, 1.30 mmole), and EDC·HCl (0.250 g, 1.30 mmole). The mixture was stirred at RT for 18 hours and water (25 mL) was added. The mixture was extracted with DCM (3×10 mL) and the combined organics were dried over magnesium sulfate, filtered, and concentrated. The residue was dissolved in DCM (4.4 mL) and triethylamine (0.170 mL, 1.21 mmole) and acetic anhydride (0.155 mL, 1.22 mmole) were added. The mixture was stirred at RT for 18 hours and water (25 mL) was added. The mixture was extracted with DCM (3×10 mL) and the combined organics were dried over magnesium sulfate, filtered, and concentrated. The crude residue was purified by silica gel chromatography to provide methyl O-acetyl-N—(N-(2-(4-((6-azidohexanamido)methyl)phenyl)thiazole-4-carbonyl)-O-(tert-butyldimethylsilyl)-L-seryl)-L-serinate as a colorless oil (0.538 g, 69%). ¹H NMR (400 MHz, CDCl₃): δ 8.27 (d, J=7.3 Hz, 1H), 8.12 (d, J=2.4 Hz, 1H), 7.96 (dd, J=8.2, 1.6 Hz, 2H), 7.49 (d, J=7.8 Hz, 1H), 7.40-7.33 (m, 2H), 5.87 (t, J=5.8 Hz, 1H), 4.91 (tt, J=7.2, 3.8 Hz, 1H), 4.68 (td, J=7.3, 3.8 Hz, 1H), 4.51 (ddt, J=10.5, 6.6, 3.5 Hz, 3H), 4.36 (dd, J=11.4, 3.6 Hz, 1H), 4.24 (ddd, J=9.8, 3.8, 2.8 Hz, 1H), 3.87-3.80 (m, 1H), 3.79 (s, 3H), 3.30 (t, J=6.8 Hz, 2H), 2.29 (t, J=7.5 Hz, 2H), 2.03 (s, 3H), 1.74 (tt, J=8.3, 6.5 Hz, 2H), 1.69-1.59 (m, 2H), 1.51-1.41 (m, 2H), 0.98 (s, 9H), 0.19 (s, 3H), 0.17 (s, 3H).

### Methyl 2-(2-(2-(4-((6-azidohexanamido)methyl)phenyl)thiazole-4-carboxamido)acrylamido)acrylate (39)

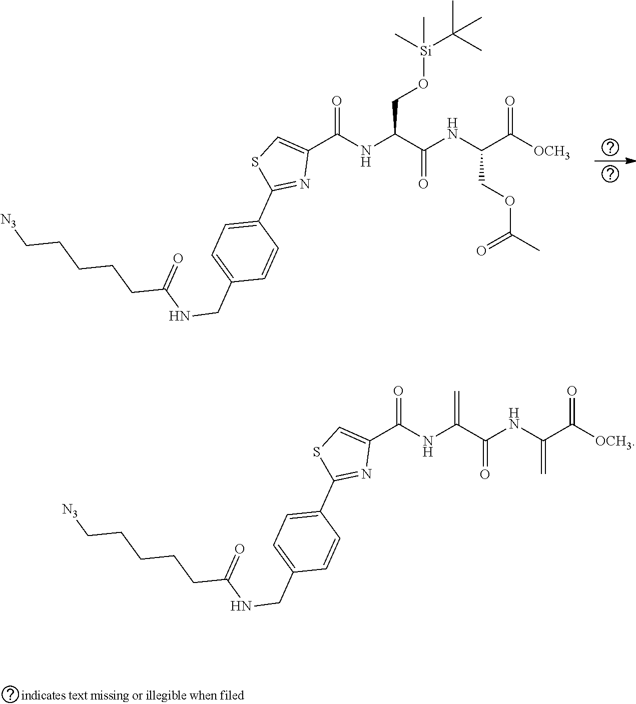 [SMILES: COC(=O)[C@H](COC(C)=O)NC(=O)[C@H](CO[Si](C)(C)C(C)(C)C)NC(=O)c1csc(-c2ccc(CNC(=O)CCCCCN=[N+]=[N-])cc2)n1 ; C=C(NC(=O)c1csc(-c2ccc(CNC(=O)CCCCCN=[N+]=[N-])cc2)n1)C(=O)NC(=C)C(=O)OC]

Methyl O-acetyl-N—(N-(2-(4-((6-azidohexanamido)methyl)phenyl)thiazole-4-carbonyl)-O-(tert-butyldimethylsilyl)-L-seryl)-L-serinate (0.538 g, 0.749 mmole) was dissolved in THF (3 mL). TBAF (1M solution in THF, 1.90 mL, 1.90 mmole) was added and the solution was stirred at RT for 2 hours. Water (25 mL) was added and the mixture was extracted with ethyl acetate (3×10 mL). The combined organics were dried over magnesium sulfate, filtered, and concentrated. The residue was dissolved in DCM (3 mL) and cooled to 0° C. Triethylamine (0.160 mL, 1.14 mmole) and methanesulfonyl chloride (0.087 mL, 1.12 mmole) were added and the mixture was stirred at 0° for 2 hours. Water (25 mL) was added and the mixture was extracted with DCM (3×10 mL). The combined organics were dried over magnesium sulfate, filtered, and concentrated. The residue was dissolved in THE (3 mL) and cooled to 0° C. DBU (0.170 mL, 1.14 mmole) was added and the mixture was stirred at 0° for 30 minutes. Water (25 mL) was added and the mixture was extracted with ethyl acetate (3×10 mL). The combined organics were dried over magnesium sulfate, filtered, and concentrated. The crude residue was purified by silica gel chromatography to provide methyl 2-(2-(2-(4-((6-azidohexanamido)methyl)phenyl)thiazole-4-carboxamido)acrylamido)acrylate as a white solid (0.160 g, 41%). ¹H NMR (400 MHz, CDCl₃): δ 10.09-9.96 (m, 1H), 8.57 (s, 1H), 8.16 (s, 1H), 8.04-7.94 (m, 2H), 7.44-7.34 (m, 2H), 6.79 (s, 1H), 6.72 (s, 1H), 6.05 (s, 1H), 5.90 (s, 1H), 5.52 (s, 1H), 4.52 (d, J=5.9 Hz, 2H), 3.92 (s, 3H), 3.30 (t, J=6.8 Hz, 2H), 2.29 (t, J=7.5 Hz, 2H), 1.80-1.69 (m, 2H), 1.68-1.59 (m, 2H), 1.43 (t, J=7.2 Hz, 2H).

### Compound 40: Methyl 2-(2-(2-(4-((6-chlorohexanamido)methyl)piperidin-1-yl)thiazole-4-carboxamido)acrylamido)acrylate

### Ethyl 2-(4-(((tert-butoxycarbonyl)amino)methyl)piperidin-1-yl)thiazole-4-carboxylate (40.1)

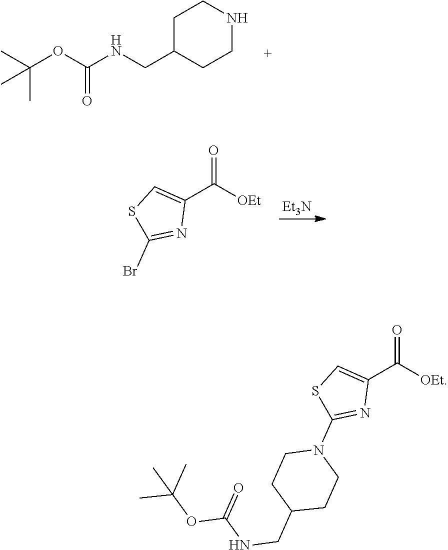 [SMILES: "Et3N" ; CC(C)(C)OC(=O)NCC1CCNCC1 ; CCOC(=O)c1csc(Br)n1 ; CCOC(=O)c1csc(N2CCC(CNC(=O)OC(C)(C)C)CC2)n1]

Tert-butyl (piperidin-4-ylmethyl)carbamate (2.500 g, 11.7 mmole) was dissolved in DMA (12 mL). Ethyl 2-bromothiazole-4-carboxylate (3.027 g, 12.8 mmole) and triethylamine (2.00 mL, 14.3 mmole) were added and the mixture was heated to 80° C. for 20 hours, then was cooled to RT and diluted with ethyl acetate (40 mL). The mixture was washed with water (3×25 mL) and the organics were dried over magnesium sulfate, filtered, and concentrated. The crude residue was purified by silica gel chromatography to provide ethyl 2-(4-(((tert-butoxycarbonyl)amino) methyl)piperidin-1-yl)thiazole-4-carboxylate as a pale yellow oil (4.194 g). ¹H NMR (400 MHz, CDCl₃): δ 7.42 (s, 1H), 4.69 (d, J=6.4 Hz, 1H), 4.35 (q, J=7.1 Hz, 2H), 4.10-4.01 (m, 2H), 3.09-2.94 (m, 4H), 1.86-1.76 (m, 2H), 1.45 (s, 9H), 1.37 (t, J=7.1 Hz, 3H), 1.34-1.28 (m, 2H).

### Methyl (2-(4-(((tert-butoxycarbonyl)amino)methyl)piperidin-1-yl)thiazole-4-carbonyl)-L-serinate (40.2)

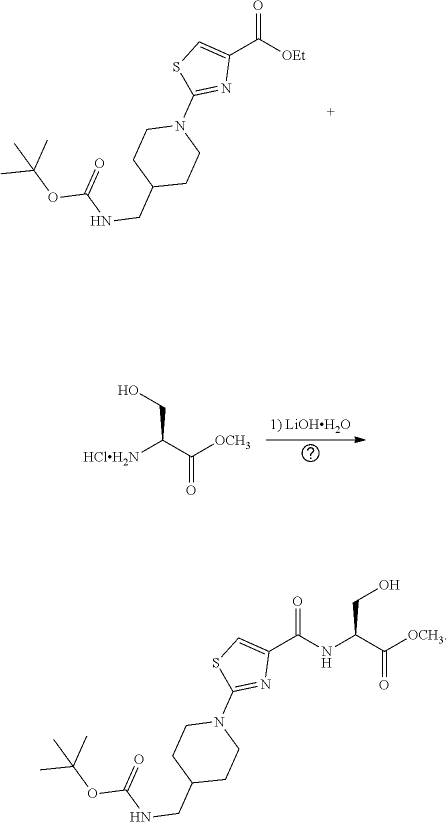

Ethyl 2-(4-(((tert-butoxycarbonyl)amino)methyl)piperidin-1-yl)thiazole-4-carboxylate (4.194 g, 11.4 mmole) was dissolved in 4/1/1 THF/methanol/water (22 mL). Lithium hydroxide monohydrate (0.715 g, 17.0 mmole) was added and the mixture was stirred at RT for 3 hours. Water (50 mL) was added and the solution was treated with 1N aq. HCl to pH=4. The mixture was extracted with ethyl acetate (3×25 mL) and the combined organics were dried over magnesium sulfate, filtered, and concentrated. The residue was dissolved in DCM (22 mL). L-Serine methyl ester hydrochloride (2.128 g, 13.7 mmole) was added followed by N,N-diisopropylethylamine (4.00 mL, 23.0 mmole), HOBt·H₂O (2.106 g, 13.8 mmole), and EDC·HCl (2.617 g, 13.7 mmole). The mixture was stirred at RT for 2¹2 days and water (50 mL) was added. The two layers were separated and the aqueous layer was extracted with DCM (2×10 mL). The combined organics were dried over magnesium sulfate, filtered, and concentrated and the crude residue was purified by silica gel chromatography to provide methyl (2-(4-(((tert-butoxycarbonyl)amino)methyl)piperidin-1-yl)thiazole-4-carbonyl)-L-serinate as a white solid (5.150 g, 99%). ¹H NMR (400 MHz, CDCl₃): δ 8.01 (d, J=7.4 Hz, 1H), 7.38 (s, 1H), 4.81 (dt, J =7.5, 3.8 Hz, 1H), 4.71 (s, 1H), 4.09-3.97 (m, 4H), 3.83 (s, 3H), 3.12-2.98 (m, 4H), 2.95 (t, J=6.2 Hz, 1H), 1.88-1.78 (m, 2H), 1.47 (s, 9H), 1.40-1.29 (m, 2H).

### Methyl O-(tert-butyldimethylsilyl)-N-(2-(4-((6-chlorohexanamido)methyl)piperidin-1-yl)thiazole-4-carbonyl)-L-serinate (40.3)

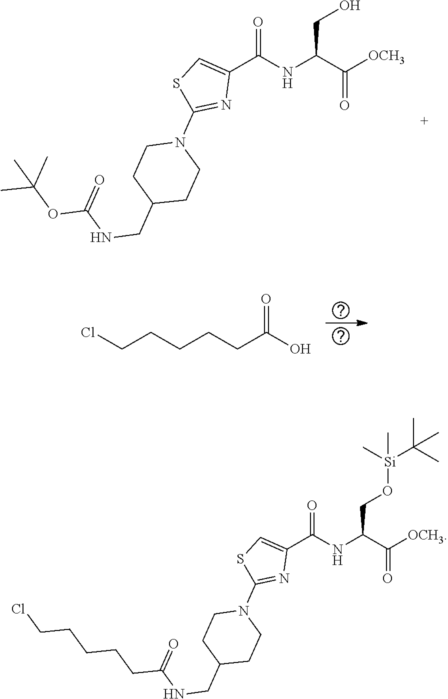 [SMILES: COC(=O)[C@H](CO)NC(=O)c1csc(N2CCC(CNC(=O)OC(C)(C)C)CC2)n1 ; O=C(O)CCCCCCl ; COC(=O)[C@H](CO[Si](C)(C)C(C)(C)C)NC(=O)c1csc(N2CCC(CNC(=O)CCCCCCl)CC2)n1]

Methyl (2-(4-(((tert-butoxycarbonyl)amino)methyl)piperidin-1-yl)thiazole-4-carbonyl)-L-serinate (0.610 g, 1.38 mmole) was dissolved in DCM (4 mL). HCl (4M solution in 1,4-dioxane, 2.70 mL, 10.8 mmole) was added and the mixture was stirred at RT for 4 hours, then was concentrated. The residue was suspended in DCM (4 mL) and 6-chlorohexanoic acid (0.251 g, 1.67 mmole) was added. N,N-Diisopropylethylamine (0.480 mL, 2.76 mmole) was added followed by HOBt·H₂O (0.253 g, 1.65 mmole) and EDC·HCl (0.315 g, 1.64 mmole), and the mixture was stirred at RT for 18 hours. Water (25 mL) was added and the mixture was extracted with DCM (3×10 mL). The combined organics were dried over magnesium sulfate, filtered, and concentrated. The residue was dissolved in DCM (4 mL). Imidazole (0.107 g, 1.57 mmole) and tert-butyldimethylchlorosilane (0.235 g, 1.56 mmole) were added and the mixture was stirred at RT for 3 days. Water (25 mL) was added and the mixture was extracted with DCM (3×10 mL). The combined organics were dried over magnesium sulfate, filtered, and concentrated. The crude residue was purified by silica gel chromatography to provide methyl O-(tert-butyldimethylsilyl)-N-(2-(4-((6-chlorohexanamido)methyl)piperidin-1-yl)thiazole-4-carbonyl)-L-serinate as a colorless oil (0.416 g, 51%). ¹H NMR (400 MHz, CDCl₃): δ 7.95 (d, J=8.8 Hz, 1H), 7.34 (s, 1H), 5.71 (t, J=6.3 Hz, 1H), 4.81-4.72 (m, 1H), 4.15 (d, J=10.2 Hz, 1H), 4.04-3.92 (m, 2H), 3.87 (dd, J=10.0, 3.4 Hz, 1H), 3.74 (s, 3H), 3.53 (t, J=6.6 Hz, 2H), 3.18 (t, J=6.2 Hz, 2H), 2.97 (tdd, J=12.7, 8.2, 2.6 Hz, 2H), 2.20 (t, J=7.5 Hz, 2H), 1.82-1.71 (m, 5H), 1.67 (tt, J =8.5, 6.5 Hz, 2H), 1.52-1.42 (m, 2H), 1.38-1.27 (m, 2H), 0.87 (s, 9H), 0.03 (s, 3H), 0.01 (s, 3H).

### Methyl O-acetyl-N—(O-(tert-butyldimethylsilyl)-N-(2-(4-((6-chlorohexanamido)methyl) piperidin-1-yl)thiazole-4-carbonyl)-L-seryl)-L-serinate (40.4)

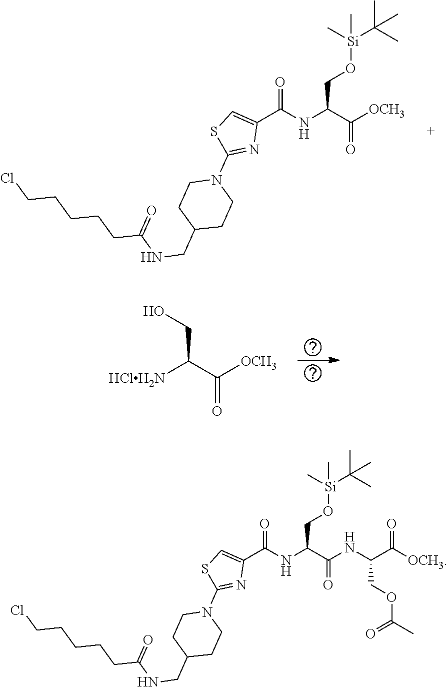

Methyl O-(tert-butyldimethylsilyl)-N-(2-(4-((6-chlorohexanamido)methyl)piperidin-1-yl)thiazole-4-carbonyl)-L-serinate (0.416 g, 0.706 mmole) was dissolved in 4/1/1 THF/methanol/water (2.8 mL). Lithium hydroxide monohydrate (0.053 g, 1.26 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered, and concentrated. The residue was dissolved in DCM (2.8 mL) and L-serine methyl ester hydrochloride (0.135 g, 0.868 mmole) was added. N,N-Diisopropylethylamine (0.250 mL, 1.44 mmole) was added followed by HOBt·H₂O (0.136 g, 0.888 mmole) and EDC·HCl (0.165 g, 0.861 mmole) and the mixture was stirred at RT for 18 hours. Water (25 mL) was added and the mixture was extracted with DCM (3×10 mL). The combined organics were dried over magnesium sulfate, filtered, and concentrated. The residue was dissolved in DCM (2.8 mL) and triethylamine (0.110 mL, 0.785 mmole) and acetic anhydride (0.074 mL, 0.783 mmole) were added. The solution was stirred at RT for 3 hours and water (25 mL) was added. The mixture was extracted with DCM (3×10 mL) and the combined organics were dried over magnesium sulfate, filtered, and concentrated. The crude residue was purified by silica gel chromatography to provide methyl O-acetyl-N—(O-(tert-butyldimethylsilyl)-N-(2-(4-((6-chlorohexanamido)methyl)piperidin-1-yl)thiazole-4-carbonyl)-L-seryl)-L-serinate as a thick, colorless oil (0.299 g, 59%). ¹H NMR (400 MHz, CDCl₃): δ 7.98 (d, J=7.1 Hz, 1H), 7.43 (d, J=7.8 Hz, 1H), 7.37 (d, J=2.7 Hz, 1H), 5.57 (t, J=6.1 Hz, 1H), 4.86 (dq, J=8.2, 4.0 Hz, 1H), 4.57 (ddd, J=7.2, 6.0, 3.6 Hz, 1H), 4.46 (ddd, J=11.8, 7.9, 4.0 Hz, 1H), 4.31 (dd, J=11.4, 3.7 Hz, 1H), 4.22-4.15 (m, 1H), 4.00 (d, J=13.0 Hz, 2H), 3.76 (d, J=2.3 Hz, 3H), 3.75-3.70 (m, 1H), 3.54 (t, J=6.6 Hz, 2H), 3.20 (t, J=6.2 Hz, 2H), 2.99 (tdd, J=12.8, 5.0, 2.7 Hz, 2H), 2.21 (t, J=7.5 Hz, 2H), 2.02 (d, J=2.7 Hz, 3H), 1.85-1.73 (m, 5H), 1.73-1.64 (m, 2H), 1.55-1.43 (m, 2H), 1.40-1.27 (m, 2H), 0.92 (s, 9H), 0.14 (s, 3H), 0.12 (s, 3H).

### Methyl 2-(2-(2-(4-((6-chlorohexanamido)methyl)piperidin-1-yl)thiazole-4-carboxamido)acrylamido)acrylate (40)

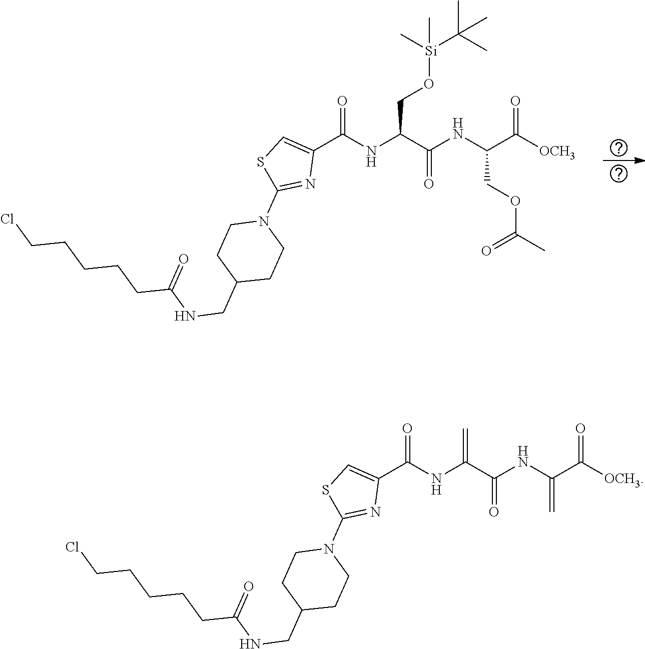 [SMILES: COC(=O)[C@H](COC(C)=O)NC(=O)[C@H](CO[Si](C)(C)C(C)(C)C)NC(=O)c1csc(N2CCC(CNC(=O)CCCCCCl)CC2)n1 ; C=C(NC(=O)c1csc(N2CCC(CNC(=O)CCCCCCl)CC2)n1)C(=O)NC(=C)C(=O)OC]

Methyl O-acetyl-N—(O-(tert-butyldimethylsilyl)-N-(2-(4-((6-chlorohexanamido)methyl) piperidin-1-yl)thiazole-4-carbonyl)-L-seryl)-L-serinate (0.299 g, 0.416 mmole) was dissolved in THF (1.6 mL). TBAF (1M solution in THF, 1.05 mL, 1.05 mmole) was added and the solution was stirred at RT for 2 hours. Water (25 mL) was added and the mixture was extracted with ethyl acetate (3×10 mL). The combined organics were dried over magnesium sulfate, filtered, and concentrated. The residue was dissolved in DCM (1.6 mL) and cooled to 0° C. Triethylamine (0.088 mL, 0.628 mmole) and methanesulfonyl chloride (0.049 mL, 0.633 mmole) were added and the mixture was stirred at 0° for 90 minutes. Water (25 mL) was added and the mixture was extracted with DCM (3×10 mL). The combined organics were dried over magnesium sulfate, filtered, and concentrated. The residue was dissolved in THE (1.6 mL) and cooled to 0° C. DBU (0.095 mL, 0.635 mmole) was added and the mixture was stirred at 0° for 60 minutes. Water (25 mL) was added and the mixture was extracted with ethyl acetate (3×10 mL). The combined organics were dried over magnesium sulfate, filtered, and concentrated. The crude residue was purified by silica gel chromatography to provide methyl 2-(2-(2-(4-((6-chlorohexanamido) methyl)piperidin-1-yl)thiazole-4-carboxamido)acrylamido)acrylate as a colorless oil (0.154 g, 70%). ¹H NMR (400 MHz, CDCl₃): δ 9.72 (s, 1H), 8.51 (s, 1H), 7.40 (s, 1H), 6.71 (d, J=2.1 Hz, 1H), 6.65 (s, 1H), 6.00 (d, J=1.3 Hz, 1H), 5.55 (t, J=6.2 Hz, 1H), 5.43 (t, J=1.9 Hz, 1H), 4.04 (d, J=12.8 Hz, 2H), 3.89 (s, 3H), 3.54 (t, J=6.6 Hz, 2H), 3.20 (t, J=6.2 Hz, 2H), 3.01 (td, J=12.6, 2.6 Hz, 2H), 2.21 (t, J=7.5 Hz, 2H), 1.79 (dtd, J=14.8, 8.2, 7.4, 3.9 Hz, 5H), 1.74-1.63 (m, 2H), 1.49 (tt, J=9.7, 5.9 Hz, 2H), 1.35 (td, J=12.2, 11.7, 4.3 Hz, 2H).

### Compound 41: Methyl 2-(2-(2-(4-((2-(2-methoxyethoxy)acetamido)methyl)piperidin-1-yl)thiazole-4-carboxamido)acrylamido)acrylate

### Methyl O-(tert-butyldimethylsilyl)-N-(2-(4-((2-(2-methoxyethoxy)acetamido)methyl)piperidin-1-yl)thiazole-4-carbonyl)-L-serinate (41.1)

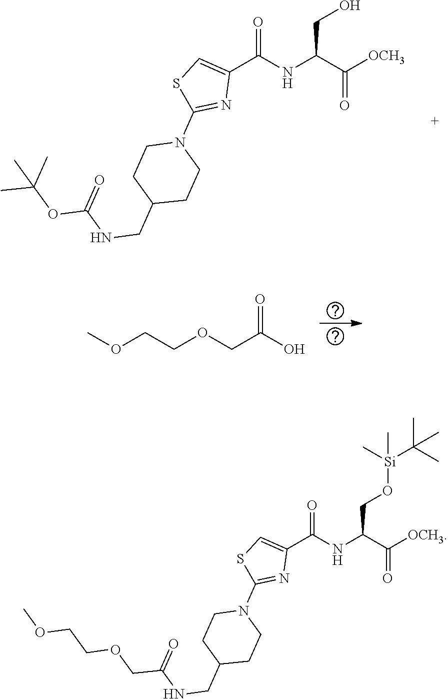

Methyl (2-(4-(((tert-butoxycarbonyl)amino)methyl)piperidin-1-yl)thiazole-4-carbonyl)-L-serinate (Example 40.2, 0.614 g, 1.39 mmole) was dissolved in DCM (2.7 mL). HCl (4M solution in 1,4-dioxane, 2.70 mL, 10.8 mmole) was added and the mixture was stirred at RT for 4 hours, then was concentrated. The residue was suspended in DCM (4 mL) and 2-(2-methoxyethoxy)acetic acid (0.235 g, 1.75 mmole) was added. N,N-Diisopropylethylamine (0.480 mL, 2.76 mmole) was added followed by HOBt·H₂O (0.252 g, 1.65 mmole) and EDC·HCl (0.319 g, 1.66 mmole), and the mixture was stirred at RT for 18 hours. Water (25 mL) was added and the mixture was extracted with DCM (3×10 mL). The combined organics were dried over magnesium sulfate, filtered, and concentrated. The residue was dissolved in DCM (2.7 mL). Imidazole (0.104 g, 1.53 mmole) and tert-butyldimethylchlorosilane (0.226 g, 1.50 mmole) were added and the mixture was stirred at RT for 18 hours. Water (25 mL) was added and the mixture was extracted with DCM (3×10 mL). The combined organics were dried over magnesium sulfate, filtered, and concentrated. The crude residue was purified by silica gel chromatography to provide methyl O-(tert-butyldimethylsilyl)-N-(2-(4-((2-(2-methoxyethoxy)acetamido)methyl)piperidin-1-yl)thiazole-4-carbonyl)-L-serinate as a thick, colorless gel (0.363 g, 46%). ¹H NMR (400 MHz, CDCl₃): δ 7.96 (d, J=8.8 Hz, 1H), 7.36 (s, 1H), 7.17 (t, J=6.2 Hz, 1H), 4.83-4.73 (m, 1H), 4.15 (dd, J=10.0, 2.6 Hz, 1H), 4.01 (s, 4H), 3.88 (dd, J=10.0, 3.4 Hz, 1H), 3.75 (s, 3H), 3.72-3.65 (m, 2H), 3.61-3.52 (m, 2H), 3.41 (s, 3H), 3.24 (t, J=6.3 Hz, 2H), 2.99 (tdd, J=12.5, 7.1, 2.6 Hz, 2H), 1.87-1.70 (m, 3H), 1.36 (dp, J=18.8, 7.2, 6.1 Hz, 2H), 0.88 (s, 9H), 0.04 (s, 3H), 0.03 (s, 3H).

### Methyl O-acetyl-N—(O-(tert-butyldimethylsilyl)-N-(2-(4-((2-(2-methoxyethoxy)acetamido)methyl)piperidin-1-yl)thiazole-4-carbonyl)-L-seryl)-L-serinate (41.2)

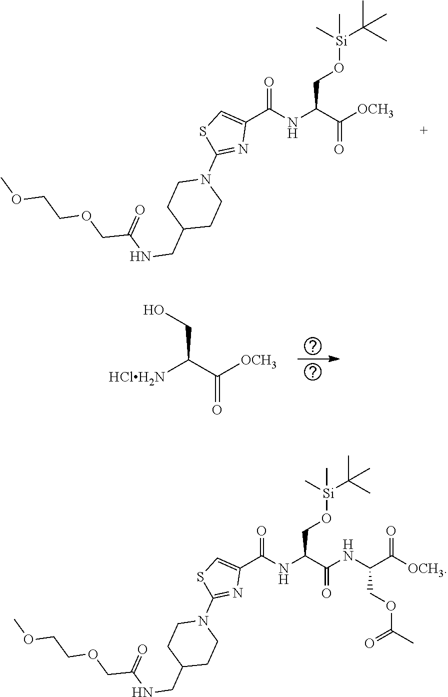

Methyl 0-(tert-butyldimethylsilyl)-N-(2-(4-((2-(2-methoxyethoxy)acetamido)methyl)piperidin-1-yl)thiazole-4-carbonyl)-L-serinate (0.363 g, 0.634 mmole) was dissolved in 4/1/1 THF/methanol/water (2.6 mL). Lithium hydroxide monohydrate (0.043 g, 1.02 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered, and concentrated. The residue was dissolved in DCM (2.6 mL) and L-serine methyl ester hydrochloride (0.125 g, 0.803 mmole) was added. N,N-Diisopropylethylamine (0.220 mL, 1.26 mmole) was added followed by HOBt·H₂O (0.122 g, 0.797 mmole) and EDC·HCl (0.149 g, 0.777 mmole) and the mixture was stirred at RT for 18 hours. Water (25 mL) was added and the mixture was extracted with DCM (3×10 mL). The combined organics were dried over magnesium sulfate, filtered, and concentrated. The residue was dissolved in DCM (2.6 mL) and triethylamine (0.100 mL, 0.714 mmole) and acetic anhydride (0.066 mL, 0.698 mmole) were added. The solution was stirred at RT for 3 hours and water (25 mL) was added. The mixture was extracted with DCM (3×10 mL) and the combined organics were dried over magnesium sulfate, filtered, and concentrated. The crude residue was purified by silica gel chromatography to provide methyl O-acetyl-N-(0-(tert-butyldimethylsilyl)-N-(2-(4-((2-(2-methoxyethoxy)acetamido)methyl)piperidin-1-yl)thiazole-4-carbonyl)-L-seryl)-L-serinate as a thick, colorless gel (0.231 g, 52%). ¹H NMR (400 MHz, CDCl₃): δ 7.99 (d, J=7.1 Hz, 1H), 7.43 (d, J=7.8 Hz, 1H), 7.37 (d, J=2.8 Hz, 1H), 7.17 (t, J=6.1 Hz, 1H), 4.87 (dt, J=7.7, 3.8 Hz, 1H), 4.58 (td, J=7.2, 3.7 Hz, 1H), 4.52-4.43 (m, 1H), 4.31 (dd, J=11.4, 3.7 Hz, 1H), 4.18 (ddd, J=9.8, 3.7, 1.7 Hz, 1H), 4.06-3.97 (m, 2H), 4.01 (s, 2H), 3.76 (s, 3H), 3.75-3.71 (m, 1H), 3.71-3.65 (m, 2H), 3.61-3.53 (m, 2H), 3.41 (s, 3H), 3.24 (t, J=6.3 Hz, 2H), 3.07-2.93 (m, 2H), 2.02 (d, J=2.5 Hz, 3H), 1.88-1.71 (m, 3H), 1.36 (p, J=12.4, 10.6 Hz, 2H), 0.92 (s, 9H), 0.14 (s, 3H), 0.12 (s, 3H).

### Methyl 2-(2-(2-(4-((2-(2-methoxyethoxy)acetamido)methyl)piperidin-1-yl)thiazole-4-carboxamido)acrylamido)acrylate (41)

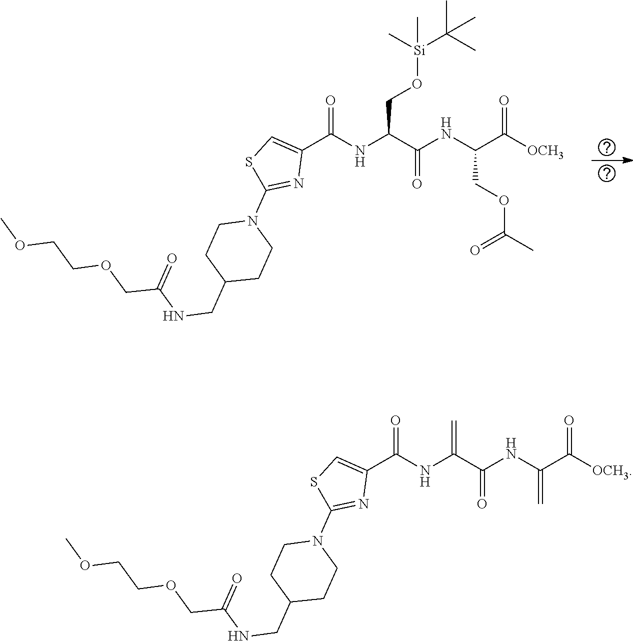 [SMILES: COCCOCC(=O)NCC1CCN(c2nc(C(=O)N[C@@H](CO[Si](C)(C)C(C)(C)C)C(=O)N[C@@H](COC(C)=O)C(=O)OC)cs2)CC1 ; C=C(NC(=O)c1csc(N2CCC(CNC(=O)COCCOC)CC2)n1)C(=O)NC(=C)C(=O)OC]

Methyl O-acetyl-N-(0-(tert-butyldimethylsilyl)-N-(2-(4-((2-(2-methoxyethoxy)acetamido)methyl)piperidin-1l-yl)thiazole-4-carbonyl)-L-seryl)-L-seri nate (0.231 g, 0.329 mmole) was dissolved in THF (1.2 mL). TBAF (1M solution in THF, 0.820 mL, 0.820 mmole) was added and the solution was stirred at RT for 2 hours. Water (25 mL) was added and the mixture was extracted with ethyl acetate (3×10 mL). The combined organics were dried over magnesium sulfate, filtered, and concentrated. The residue was dissolved in DCM (1.2 mL) and cooled to 0° C. Triethylamine (0.070 mL, 0.499 mmole) and methanesulfonyl chloride (0.039 mL, 0.504 mmole) were added and the mixture was stirred at 0° for 90 minutes. Water (25 mL) was added and the mixture was extracted with DCM (3×10 mL). The combined organics were dried over magnesium sulfate, filtered, and concentrated. The residue was dissolved in THF (1.2 mL) and cooled to 0° C. DBU (0.074 mL, 0.495 mmole) was added and the mixture was stirred at 0° for 60 minutes. Water (25 mL) was added and the mixture was extracted with ethyl acetate (3×10 mL). The combined organics were dried over magnesium sulfate, filtered, and concentrated. The crude residue was purified by silica gel chromatography to provide methyl 2-(2-(2-(4-((2-(2-methoxyethoxy)acetamido)methyl)piperidin-1l-yl)thiazole-4-carboxamido)acrylamido)acrylate as a colorless oil (0.154 g). ¹H NMR (400 MHz, CDCl₃): δ 9.75 (s, 1H), 8.53 (s, 1H), 7.43 (s, 1H), 7.20 (d, J=6.4 Hz, 1H), 6.74 (d, J=2.1 Hz, 1H), 6.68 (s, 1H), 6.02 (d, J=1.3 Hz, 1H), 5.45 (t, J=1.9 Hz, 1H), 4.07 (d, J=12.9 Hz, 2H), 4.04 (s, 2H), 3.91 (s, 3H), 3.75-3.67 (m, 2H), 3.63-3.56 (m, 2H), 3.44 (s, 3H), 3.27 (t, J=6.4 Hz, 2H), 3.04 (td, J=12.6, 2.7 Hz, 2H), 1.88 (d, J=18.9 Hz, 2H), 1.83-1.73 (m, 1H), 1.46-1.33 (m, 2H).

### Compound 42: Methyl 2-(2-(2-(4-((((3-methoxypropyl)carbamoyl)oxy)methyl)phenyl)thiazole-4-carboxamido)acrylamido)acrylate

### 4-Cyanobenzyl (3-methoxypropyl)carbamate (42.1)

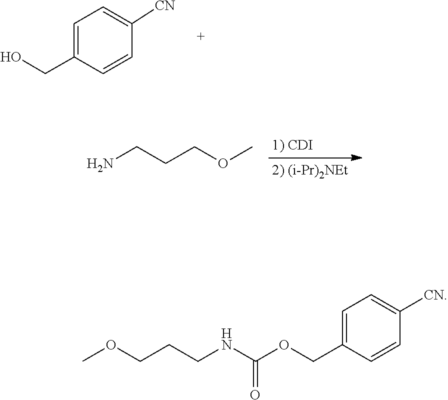

4-(Hydroxymethyl)benzonitrile (1.504 g, 11.3 mmole) was dissolved in THE (22 mL). N,N′-Carbonyldiimidazole (2.177 g, 13.4 mmole) was added and the solution was stirred at RT for 18 hours. Water (50 mL) was added and the mixture was extracted with ethyl acetate (3×15 mL). The combined organics were washed sequentially with water (2×25 mL) and sat. aq. sodium chloride (1×25 mL), then were dried over magnesium sulfate, filtered, and concentrated. The residue was dissolved in DMF (22 mL). 3-Methoxypropan-1-amine (1.30 mL, 12.7 mmole) and N,N-diisopropylethylamine (2.15 mL, 12.3 mmole) were added and the solution was heated to 70° C. for 3 hours. The mixture was cooled to RT and water (50 mL) was added. The mixture was extracted with ethyl acetate (3×15 mL) and the combined organics were washed sequentially with water (2×25 mL) and sat. aq. sodium chloride (1×25 mL). The organics were dried over magnesium sulfate, filtered and concentrated to provide 4-cyanobenzyl (3-methoxypropyl)carbamate as a pale yellow oil (2.774 g, 99%). ¹H NMR (400 MHz, CDCl₃): δ 7.71-7.61 (m, 2H), 7.46 (d, J=8.0 Hz, 2H), 5.30 (s, 1H), 5.15 (s, 2H), 3.48 (t, J=5.8 Hz, 2H), 3.39-3.28 (m, 5H), 1.80 (p, J=6.0 Hz, 2H).

### 4-Carbamothioylbenzyl (3-methoxypropyl)carbamate (42.2)

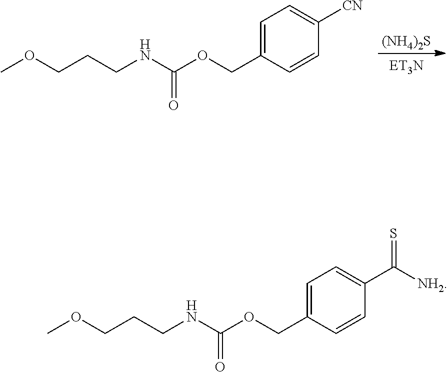 [SMILES: COCCCNC(=O)OCc1ccc(C#N)cc1 ; "(NH4)2S" ; COCCCNC(=O)OCc1ccc(C(N)=S)cc1]

4-Cyanobenzyl (3-methoxypropyl)carbamate (2.774 g, 11.2 mmole) was dissolved in pyridine (11 mL). Triethylamine (1.70 mL, 12.1 mmole) and ammonium sulfide (40% aqueous solution, 2.30 mL, 13.5 mmole) were added and the mixture was warmed to 50° C. for 90 minutes, then was cooled to RT and concentrated. The residue was dissolved in ethyl acetate (15 mL) and water (25 mL) and 1N aq. HCl (20 mL) were added. The two layers were separated and the aqueous layer was extracted with ethyl acetate (2×15 mL). The combined organics were dried over magnesium sulfate, filtered, and concentrated to provide 4-carbamothioylbenzyl (3-methoxypropyl)carbamate as a yellow solid (3.371 g). ¹H NMR (400 MHz, CDCl₃): δ 9.86 (s, 1H), 9.49 (s, 1H), 7.93-7.83 (m, 2H), 7.40-7.32 (m, 2H), 7.29 (t, J=5.7 Hz, 1H), 5.05 (s, 2H), 3.31 (t, J=6.3 Hz, 2H), 3.21 (s, 3H), 3.04 (q, J=6.8 Hz, 2H), 1.63 (p, J=6.6 Hz, 2H).

### Ethyl 2-(4-((((3-methoxypropyl)carbamoyl)oxy)methyl)phenyl)thiazole-4-carboxylate (42.3)

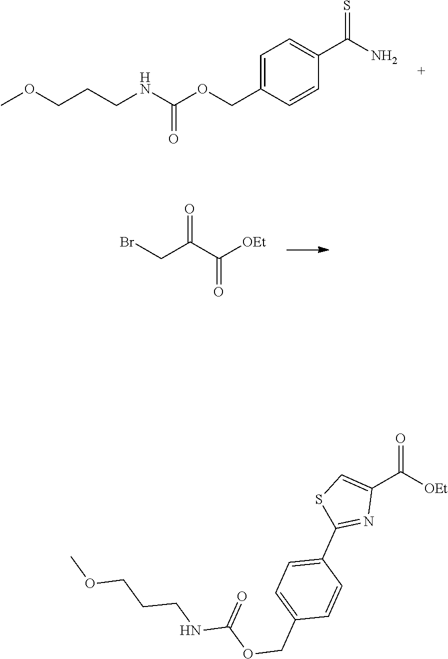 [SMILES: COCCCNC(=O)OCc1ccc(C(N)=S)cc1 ; CCOC(=O)C(=O)CBr ; CCOC(=O)c1csc(-c2ccc(COC(=O)NCCCOC)cc2)n1]

4-Carbamothioylbenzyl (3-methoxypropyl)carbamate (3.371 g, 11.9 mmole) was dissolved in ethanol (24 mL). Ethyl bromopyruvate (1.80 mL, 14.3 mmole) was added and the mixture was heated to 80° C. for 3 hours, then was cooled to RT and concentrated. The crude residue was purified by silica gel chromatography to provide ethyl 2-(4-((((3-methoxypropyl)carbamoyl)oxy)methyl)phenyl)thiazole-4-carboxylate as a yellow solid (1.435 g, 32%). ¹H NMR (400 MHz, CDCl₃): δ 8.15 (s, 1H), 8.03-7.92 (m, 2H), 7.43 (d, J=8.2 Hz, 2H), 5.21 (s, 1H), 5.12 (s, 2H), 4.44 (q, J=7.1 Hz, 2H), 3.45 (t, J=5.9 Hz, 2H), 3.36-3.27 (m, 2H), 3.32 (s, 3H), 1.79 (q, J=6.1 Hz, 2H), 1.42 (t, J=7.1 Hz, 3H).

### Methyl O-(tert-butyldimethylsilyl)-N-(2-(4-((((3-methoxypropyl)carbamoyl)oxy)methyl)phenyl)thiazole-4-carbonyl)-L-serinate (42.4)

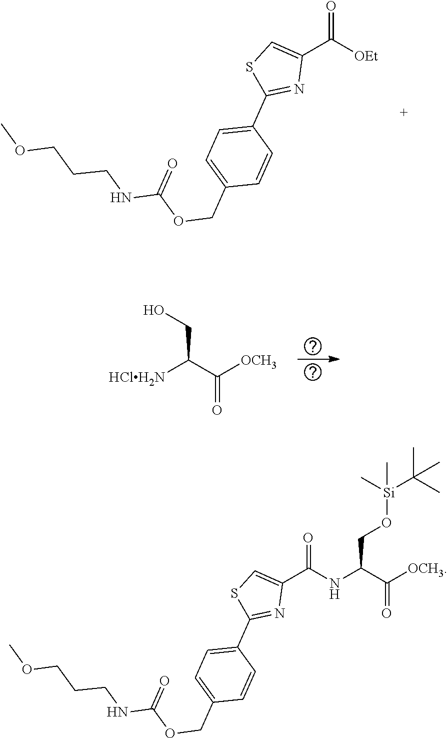

Ethyl 2-(4-((((3-methoxypropyl)carbamoyl)oxy)methyl)phenyl)thiazole-4-carboxylate (0.702 g, 1.85 mmole) was dissolved in 4/1/1 THF/methanol/water (3.6 mL). Lithium hydroxide monohydrate (0.118 g, 2.81 mmole) was added and the mixture was stirred at RT for 3 hours. Water (25 mL) was added and the solution was treated with 1N aq. HCl to pH=4. The mixture was extracted with ethyl acetate (3×20 mL) and the combined organics were dried over magnesium sulfate, filtered and concentrated. The residue was suspended in DCM (7.2 mL) and L-serine methyl ester hydrochloride (0.357 g, 2.29 mmole) was added. N,N-diisopropylethylamine (0.650 mL, 3.73 mmole) and pyBOP (1.064 g, 2.04 mmole) were added and the mixture was stirred at RT for 18 hours. Water (25 mL) was added and the mixture was extracted with DCM (3×10 mL). The combined organics were dried over magnesium sulfate, filtered, and concentrated. The residue was dissolved in DCM (7.2 mL) and imidazole (0.145 g, 2.13 mmole) and tert-butyldimethylchlorosilane (0.317 g, 2.10 mmole) were added. The mixture was stirred at RT for 18 hours and water (25 mL) was added. The mixture was extracted with DCM (3×10 mL) and the combined organics were dried over magnesium sulfate, filtered, and concentrated. The crude residue was purified by silica gel chromatography to provide methyl O-(tert-butyldimethylsilyl)-N-(2-(4-((((3-methoxypropyl)carbamoyl)oxy)methyl)phenyl)thiazole-4-carbonyl)-L-serinate as a yellow oil (0.842 g, 80%). ¹H NMR (400 MHz, CDCl₃): δ 8.22 (d, J=8.7 Hz, 1H), 8.10 (s, 1H), 7.98-7.90 (m, 2H), 7.47-7.39 (m, 2H), 5.21 (d, J=20.6 Hz, 1H), 5.13 (s, 2H), 4.85 (dt, J=8.7, 3.0 Hz, 1H), 4.20 (dd, J=10.1, 2.6 Hz, 1H), 3.94 (dd, J=10.1, 3.4 Hz, 1H), 3.78 (s, 3H), 3.46 (t, J=5.8 Hz, 2H), 3.32 (d, J=4.8 Hz, 5H), 1.83-1.72 (m, 2H), 0.91 (s, 9H), 0.07 (s, 3H), 0.06 (s, 3H).

### Methyl O-acetyl-N—(O-(tert-butyldimethylsilyl)-N-(2-(4-((((3-methoxypropyl)carbamoyl)oxy)methyl)phenyl)thiazole-4-carbonyl)-L-seryl)-L-serinate (42.5)

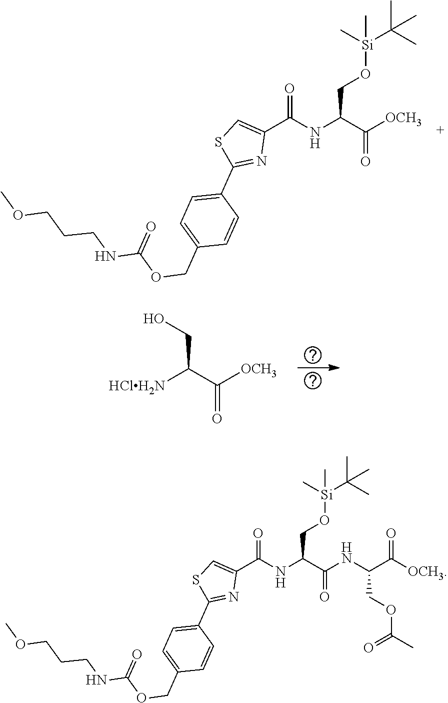

Methyl O-(tert-butyldimethylsilyl)-N-(2-(4-((((3-methoxypropyl)carbamoyl)oxy)methyl)phenyl)thiazole-4-carbonyl)-L-serinate (0.842 g, 1.49 mmole) was dissolved in 4/1/1 THF/methanol/water (3 mL). Lithium hydroxide monohydrate (0.131 g, 3.12 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered, and concentrated. The residue was dissolved in DCM (3 mL) and L-serine methyl ester hydrochloride (0.284 g, 1.83 mmole) was added, followed by N,N-diisopropylethylamine (0.520 mL, 2.99 mmole) and EDC·HCl (0.342 g, 1.78 mmole). The mixture was stirred at RT for 18 hours and water (25 mL) was added. The mixture was extracted with DCM (3×10 mL) and the combined organics were dried over magnesium sulfate, filtered, and concentrated. The residue was dissolved in DCM (3 mL) and triethylamine (0.230 ml, 1.64 mmole) and acetic anhydride (0.170 mL, 1.80 mmole) were added. The mixture was stirred at RT for 20 hours and water (25 mL) was added. The mixture was extracted with DCM (3×10 mL) and the combined organics were dried over magnesium sulfate, filtered, and concentrated. The crude residue was purified by silica gel chromatography to provide methyl O-acetyl-N-(0-(tert-butyldimethylsilyl)-N-(2-(4-((((3-methoxypropyl)carbamoyl)oxy)methyl)phenyl)thiazole-4-carbonyl)-L-seryl)-L-serinate as a colorless oil (0.693 g, 67%). ¹H NMR (400 MHz, CDCl₃): δ 8.30-8.21 (m, 1H), 8.11 (d, J=2.5 Hz, 1H), 7.95 (dq, J=8.6, 1.9 Hz, 2H), 7.44 (dd, J=11.6, 7.9 Hz, 3H), 5.22 (s, 1H), 5.14 (s, 2H), 4.89 (dq, J=7.2, 3.6 Hz, 1H), 4.66 (td, J=7.3, 3.7 Hz, 1H), 4.48 (ddd, J=11.1, 7.1, 4.0 Hz, 1H), 4.33 (dd, J=11.4, 3.6 Hz, 1H), 4.22 (dt, J=9.9, 3.6 Hz, 1H), 3.84-3.78 (m, 1H), 3.76 (d, J=4.0 Hz, 3H), 3.46 (t, J=5.8 Hz, 2H), 3.32 (d, J=5.1 Hz, 5H), 2.00 (s, 3H), 1.79 (p, J=6.0 Hz, 2H), 0.95 (s, 9H), 0.18-0.12 (m, 6H).

### Methyl 2-(2-(2-(4-((((3-methoxypropyl)carbamoyl)oxy)methyl)phenyl)thiazole-4-carboxamido)acrylamido)acrylate (42)

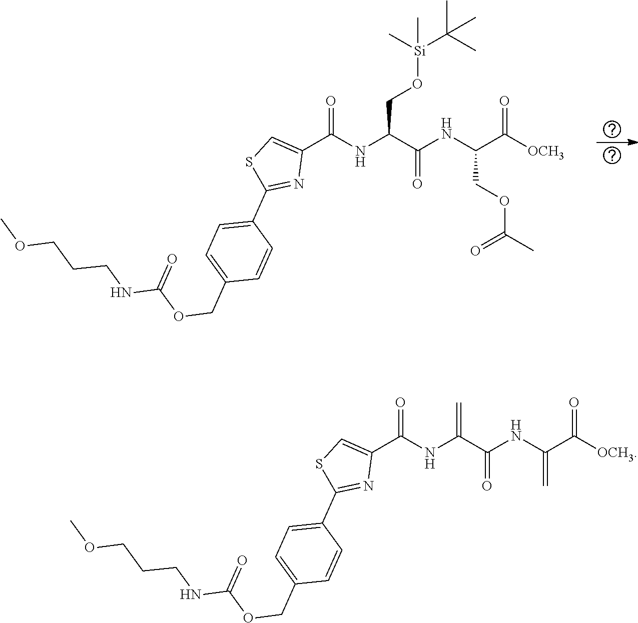 [SMILES: COCCCNC(=O)OCc1ccc(-c2nc(C(=O)N[C@@H](CO[Si](C)(C)C(C)(C)C)C(=O)N[C@@H](COC(C)=O)C(=O)OC)cs2)cc1 ; C=C(NC(=O)c1csc(-c2ccc(COC(=O)NCCCOC)cc2)n1)C(=O)NC(=C)C(=O)OC]

Methyl O-acetyl-N-(0-(tert-butyldimethylsilyl)-N-(2-(4-((((3-methoxypropyl)carbamoyl)oxy)methyl)phenyl)thiazole-4-carbonyl)-L-seryl)-L-serinate (0.693 g, 0.997 mmole) was dissolved in THE (2 mL). TBAF (1M solution in THF, 2.50 mL, 2.50 mmole) was added and the mixture was stirred at RT for 3 hours. Water (25 mL) was added and the mixture was extracted with ethyl acetate (3×10 mL). The combined organics were dried over magnesium sulfate, filtered and concentrated. The residue was dissolved in DCM (2 mL) and cooled to 0° C. Triethylamine (0.210 mL, 1.50 mmole) and methanesulfonyl chloride (0.115 mL, 1.49 mmole) were added and the mixture was stirred at 0° for 45 minutes. Water (25 mL) was added and the mixture was extracted with DCM (3×10 mL). The combined organics were dried over magnesium sulfate, filtered and concentrated. The residue was dissolved in THE (2 mL) and cooled to 0° C. DBU (0.220 mL, 1.47 mmole) was added and the mixture was stirred at 0° C. for 45 minutes. Water (25 mL) was added and the mixture was extracted with ethyl acetate (3×10 mL). The combined organics were dried over magnesium sulfate, filtered and concentrated. The crude residue was purified by silica gel chromatography to provide methyl 2-(2-(2-(4-((((3-methoxypropyl)carbamoyl)oxy)methyl)phenyl)thiazole-4-carboxamido)acrylamido)acrylate as a white solid (0.193 g, 39%). ¹H NMR (400 MHz, CDCl₃): δ 10.02 (s, 1H), 8.55 (s, 1H), 8.15 (s, 1H), 8.05-7.93 (m, 2H), 7.51-7.41 (m, 2H), 6.78 (d, J=2.2 Hz, 1H), 6.71 (s, 1H), 6.03 (d, J=1.4 Hz, 1H), 5.50 (t, J=1.9 Hz, 1H), 5.21 (s, 1H), 5.14 (s, 2H), 3.90 (s, 3H), 3.47 (t, J=5.8 Hz, 2H), 3.33 (d, J=5.5 Hz, 5H), 1.79 (p, J=6.1 Hz, 2H).

### Compound 43: Methyl 2-(2-(2-(3-(((tert-butoxycarbonyl)amino)methyl)phenyl)thiazole-4-carboxamido)acrylamido)acrylate

### 3-((1,3-Dioxoisoindolin-2-yl)methyl)benzonitrile (43.1)

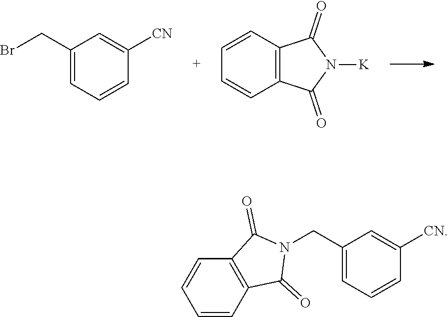

3-Cyanobenzyl bromide (2.003 g, 10.2 mmole) was dissolved in DMF (10 mL) and potassium phthalimide (2.084 g, 11.3 mmole) was added. The mixture was stirred at RT for 4 hours and water (50 mL) was added. The precipitated solid was collected by filtration, washed with water, and dried under vacuum to provide 3-((1,3-dioxoisoindolin-2-yl)methyl)benzonitrile as a white solid (2.511 g, 94%). ¹H NMR (400 MHz, CDCl₃): δ 7.87 (dd, J=5.5, 3.1 Hz, 2H), 7.79-7.69 (m, 3H), 7.67 (dt, J=7.8, 1.5 Hz, 1H), 7.57 (dt, J=7.7, 1.4 Hz, 1H), 7.44 (t, J=7.8 Hz, 1H), 4.86 (s, 2H).

### 3-((1,3-Dioxoisoindolin-2-yl)methyl)benzothioamide (43.2)

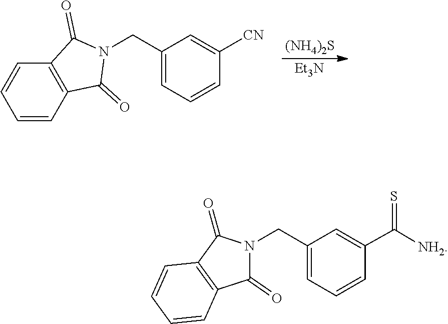

3-((1,3-Dioxoisoindolin-2-yl)methyl)benzonitrile (2.511 g, 9.57 mmole) was dissolved in pyridine. Triethylamine (1.50 mL, 10.7 mmole) and ammonium sulfide (40% aqueous solution, 2.00 mL, 11.7 mmole) were added and the mixture was warmed to 50° C. for 60 minutes, then was cooled to RT and concentrated. The residue was washed with 0.5N aq. HCl (40 mL) and the solid was collected by filtration, washed with water and diethyl ether, and allowed to air dry to provide 3-((1,3-dioxoisoindolin-2-yl)methyl)benzothioamide as a yellow solid (3.145 g). ¹H NMR (400 MHz, d₆-DMSO): δ 9.82 (s, 1H), 9.51 (s, 1H), 8.59 (m, 1H), 7.91-7.83 (m, 3H), 7.79-7.74 (m, 2H), 7.48-7.30 (m, 2H), 4.78 (s, 2H).

### Ethyl 2-(3-((1,3-dioxoisoindolin-2-yl)methyl)phenyl)thiazole-4-carboxylate (43.3)

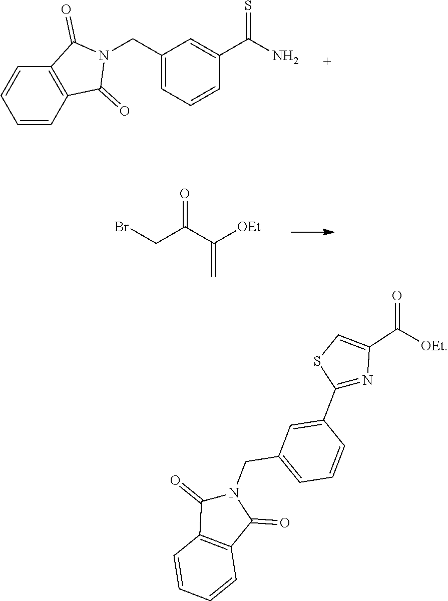

3-((1,3-Dioxoisoindolin-2-yl)methyl)benzothioamide (3.145 g, 10.6 mmole) was dissolved in ethanol (21 mL). Ethyl bromopyruvate (1.60 mL, 12.8 mmole) was added and the mixture was heated to 80° C. for 3 hours, then was cooled to RT. The precipitated solid was collected by filtration to provide ethyl 2-(3-((1,3-dioxoisoindolin-2-yl)methyl)phenyl)thiazole-4-carboxylate as a white solid (2.928 g, 78% from TLS-023-10-1). ¹H NMR (400 MHz, d₆-DMSO): δ8.57 (s, 1H), 8.01-7.95 (m, 1H), 7.95-7.82 (m, 5H), 7.55-7.43 (m, 2H), 4.88 (s, 2H), 4.34 (q, J=7.1 Hz, 2H), 1.33 (t, J=7.1 Hz, 3H).

### Ethyl 2-(3-(((tert-butoxycarbonyl)amino)methyl)phenyl)thiazole-4-carboxylate (43.4)

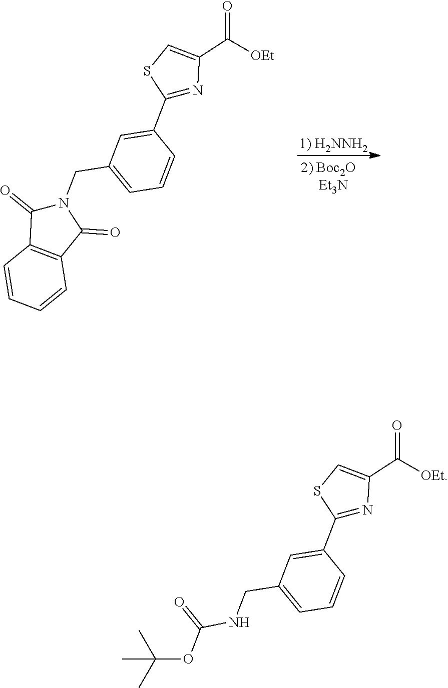 [SMILES: CCOC(=O)c1csc(-c2cccc(CN3C(=O)c4ccccc4C3=O)c2)n1 ; CCOC(=O)c1csc(-c2cccc(CNC(=O)OC(C)(C)C)c2)n1]

Ethyl 2-(3-((1,3-dioxoisoindolin-2-yl)methyl)phenyl)thiazole-4-carboxylate (2.928 g, 7.46 mmole) was suspended in ethanol (15 mL). Hydrazine (1M solution in THF, 15.0 mL, 15.0 mmole) was added and the mixture was heated to 80° C. for 2 hours, then was cooled to RT. The precipitated solid was filtered and washed with ethanol and the combined filtrates were concentrated. The residue was dissolved in DCM (15 mL) and triethylamine (1.25 mL, 8.92 mmole) and di-tert-butyl dicarbonate (1.788 g, 8.19 mmole) were added. The mixture was stirred at RT for 4 hours and water (50 mL) was added. The two layers were separated and the aqueous layer was extracted with DCM (2×10 mL) and the combined organics were dried over magnesium sulfate, filtered, and concentrated. The crude residue was purified by silica gel chromatography to provide ethyl 2-(3-(((tert-butoxycarbonyl)amino)methyl)phenyl)thiazole-4-carboxylate as a white solid (1.900 g, 70%). ¹H NMR (400 MHz, CDCl₃): δ 8.15 (s, 1H), 7.94 (d, J=2.0 Hz, 1H), 7.87 (dt, J=7.0, 2.0 Hz, 1H), 7.45-7.35 (m, 2H), 4.95 (s, 1H), 4.45 (q, J=7.1 Hz, 2H), 4.38 (d, J=6.1 Hz, 2H), 1.47 (s, 9H), 1.43 (t, J=7.1 Hz, 3H).

### Methyl N-(2-(3-(((tert-butoxycarbonyl)amino)methyl)phenyl)thiazole-4-carbonyl)-O-(tert-butyldimethylsilyl)-L-serinate (43.5)

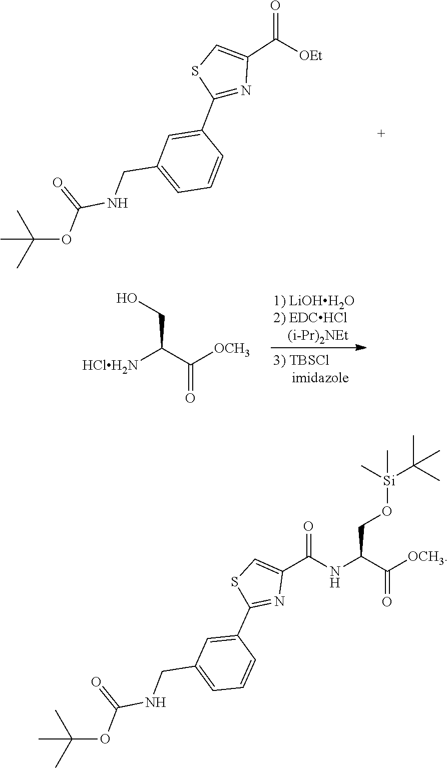

Ethyl 2-(3-(((tert-butoxycarbonyl)amino)methyl)phenyl)thiazole-4-carboxylate (0.500 g, 1.38 mmole) was dissolved in 4/1/1 THF/methanol/water (2.8 mL). Lithium hydroxide monohydrate (0.118 g, 2.81 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered, and concentrated. The residue was dissolved in DCM (5.6 mL) and L-serine methyl ester hydrochloride (0.260 g, 1.67 mmole) was added, followed by N,N-diisopropylethylamine (0.480 mL, 2.76 mmole) and EDC·HCl (0.325 g, 1.70 mmole). The mixture was stirred at RT for 3 hours and water (25 mL) was added. The mixture was extracted with DCM (3×10 mL) and the combined organics were dried over magnesium sulfate, filtered, and concentrated. The residue was dissolved in DCM (5.6 mL) and imidazole (0.108 g, 1.59 mmole) and tert-butyldimethylchlorosilane (0.230 g, 1.53 mmole) were added. The mixture was stirred at RT for 3 hours and water (25 mL) was added. The mixture was extracted with DCM (3×10 mL) and the combined organics were dried over magnesium sulfate, filtered, and concentrated. The crude residue was purified by silica gel chromatography to provide methyl N-(2-(3-(((tert-butoxycarbonyl)amino)methyl)phenyl)thiazole-4-carbonyl)-O-(tert-butyldimethylsilyl)-L-serinate as a colorless oil (0.522 g, 69%). ¹H NMR (400 MHz, CDCl₃): δ 8.19 (d, J=8.8 Hz, 1H), 8.10 (s, 1H), 7.88 (dt, J=6.7, 2.0 Hz, 1H), 7.83 (dq, J=1.8, 0.9 Hz, 1H), 7.45-7.38 (m, 2H), 4.94 (s, 1H), 4.89-4.81 (m, 1H), 4.38 (d, J=6.0 Hz, 2H), 4.20 (dd, J=10.0, 2.7 Hz, 1H), 3.95 (dd, J=10.1, 3.4 Hz, 1H), 3.78 (s, 3H), 1.47 (s, 9H), 0.91 (s, 9H), 0.07 (s, 3H), 0.05 (s, 3H).

### Methyl O-acetyl-N—(N-(2-(3-(((tert-butoxycarbonyl)amino)methyl)phenyl)thiazole-4-carbonyl)-O-(tert-butyldimethylsilyl)-L-seryl)-L-serinate (43.6)

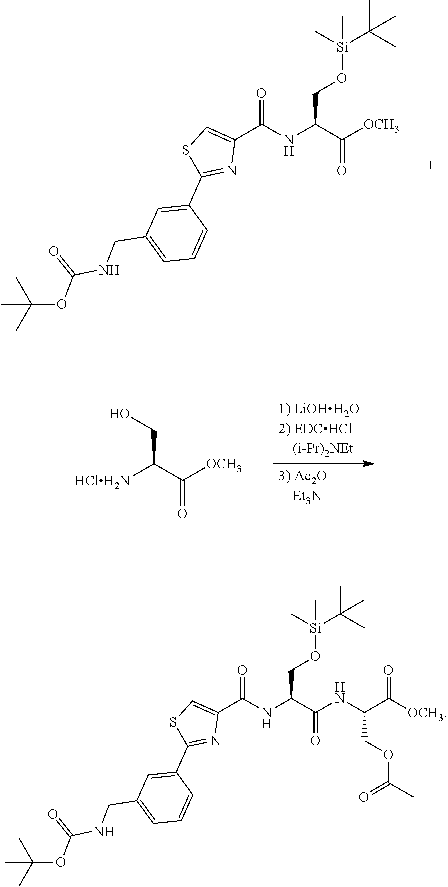

Methyl N-(2-(3-(((tert-butoxycarbonyl)amino)methyl)phenyl)thiazole-4-carbonyl)-O-(tert-butyldimethylsilyl)-L-serinate (0.522 g, 0.950 mmole) was dissolved in 4/1/1 THF/methanol/water (3 mL). Lithium hydroxide monohydrate (0.063 g, 1.50 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered, and concentrated. The residue was dissolved in DCM (3 mL). L-Serine methyl ester hydrochloride (0.177 g, 1.14 mmole) was added followed by N,N-diisopropylethylamine (0.330 mL, 1.89 mmole) and EDC·HCl (0.218 g, 1.14 mmole). The mixture was stirred at RT for 18 hours and water (25 mL) was added. The mixture was extracted with DCM (3×10 mL) and the combined organics were dried over magnesium sulfate, filtered and concentrated. The residue was dissolved in DCM (3 mL). Triethylamine (0.150 mL, 1.07 mmole) and acetic anhydride (0.100 mL, 1.06 mmole) were added and the mixture was stirred at RT for 3 hours. Water (25 mL) was added and the mixture was extracted with DCM (3×10 mL). The combined organics were dried over magnesium sulfate, filtered and concentrated. The crude residue was purified by silica gel chromatography to provide methyl O-acetyl-N—(N-(2-(3-(((tert-butoxycarbonyl)amino)methyl)phenyl)thiazole-4-carbonyl)-O-(tert-butyldimethylsilyl)-L-seryl)-L-serinate as a white solid (0.266 g, 41%). H NMR (400 MHz, CDCl₃): δ 8.23 (dd, J=7.2, 2.6 Hz, 1H), 8.12 (d, J=2.1 Hz, 1H), 7.89 (dt, J=6.4, 2.1 Hz, 1H), 7.86-7.81 (m, 1H), 7.50-7.44 (m, 1H), 7.41 (td, J=6.7, 5.4, 3.2 Hz, 2H), 4.95 (s, 1H), 4.90 (dq, J=6.9, 3.5 Hz, 1H), 4.67 (td, J=7.3, 3.8 Hz, 1H), 4.53-4.44 (m, 1H), 4.43-4.37 (m, 2H), 4.33 (dd, J=11.4, 3.6 Hz, 1H), 4.21 (ddd, J=9.9, 3.8, 2.4 Hz, 1H), 3.84-3.79 (m, 1H), 3.77 (s, 3H), 2.01 (s, 3H), 1.47 (s, 9H), 0.95 (s, 9H), 0.16 (s, 3H), 0.15 (s, 3H).

### Methyl2-(2-(2-(3-(((tert-butoxycarbonyl)amino)methyl)phenyl)thiazole-4-carboxamido)acrylamido)acrylate (43)

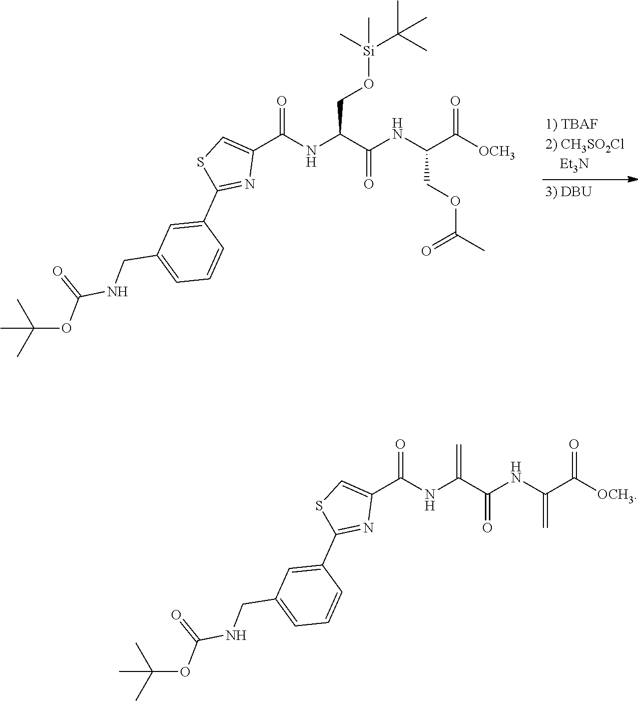 [SMILES: COC(=O)[C@H](COC(C)=O)NC(=O)[C@H](CO[Si](C)(C)C(C)(C)C)NC(=O)c1csc(-c2cccc(CNC(=O)OC(C)(C)C)c2)n1 ; C=C(NC(=O)c1csc(-c2cccc(CNC(=O)OC(C)(C)C)c2)n1)C(=O)NC(=C)C(=O)OC]

Methyl O-acetyl-N—(N-(2-(3-(((tert-butoxycarbonyl)amino)methyl)phenyl)thiazole-4-carbonyl)-O-(tert-butyldimethylsilyl)-L-seryl)-L-serinate (0.266 g, 0.392 mmole) was dissolved in THE (2 mL). TBAF (1M solution in THF, 0.980 mL, 0.980 mmole) was added and the solution was stirred at RT for 3 hours. Water (25 mL) was added and the mixture was extracted with ethyl acetate (3×10 mL). The combined organics were dried over magnesium sulfate, filtered and concentrated. The residue was dissolved in DCM (2 mL) and cooled to 0° C. Triethylamine (0.083 mL, 0.592 mmole) and methanesulfonyl chloride (0.046 mL, 0.594 mmole) were added and the mixture was stirred at 0° for 60 minutes. Water (25 mL) was added and the mixture was extracted with DCM (3×10 mL). The combined organics were dried over magnesium sulfate, filtered and concentrated. The residue was dissolved in THE (2 mL) and cooled to 0° C. DBU (0.090 mL, 0.602 mmole) was added and the mixture was stirred at 0° for 60 minutes. Water (25 mL) was added and the mixture was extracted with ethyl acetate (3×10 mL). The combined organics were dried over magnesium sulfate, filtered and concentrated. The crude residue was purified by silica gel chromatography to provide methyl 2-(2-(2-(3-(((tert-butoxycarbonyl)amino)methyl)phenyl)thiazole-4-carboxamido)acrylamido)acrylate as a white solid (0.088 g, 46%). ¹H NMR (400 MHz, CDCl₃): δ 9.98 (s, 1H), 8.55 (s, 1H), 8.16 (s, 1H), 7.94 (dt, J=7.3, 1.7 Hz, 1H), 7.86 (td, J=1.8, 0.8 Hz, 1H), 7.48-7.38 (m, 2H), 6.78 (d, J=2.2 Hz, 1H), 6.72 (s, 1H), 6.03 (d, J=1.3 Hz, 1H), 5.51 (t, J=1.9 Hz, 1H), 4.97 (s, 1H), 4.41 (d, J=6.1 Hz, 2H), 3.90 (s, 3H), 1.48 (s, 9H).

### Compound 44: Methyl 2-(2-(2-(4-((6-((tert-butoxycarbonyl)amino)hexanamido)methyl)phenyl)thiazole-4-carboxamido)acrylamido)acrylate

### Methyl N-(2-(4-((6-((tert-butoxycarbonyl)amino)hexanamido)methyl)phenyl)thiazole-4-carbonyl)-O-(tert-butyldimethylsilyl)-L-serinate (44.1)

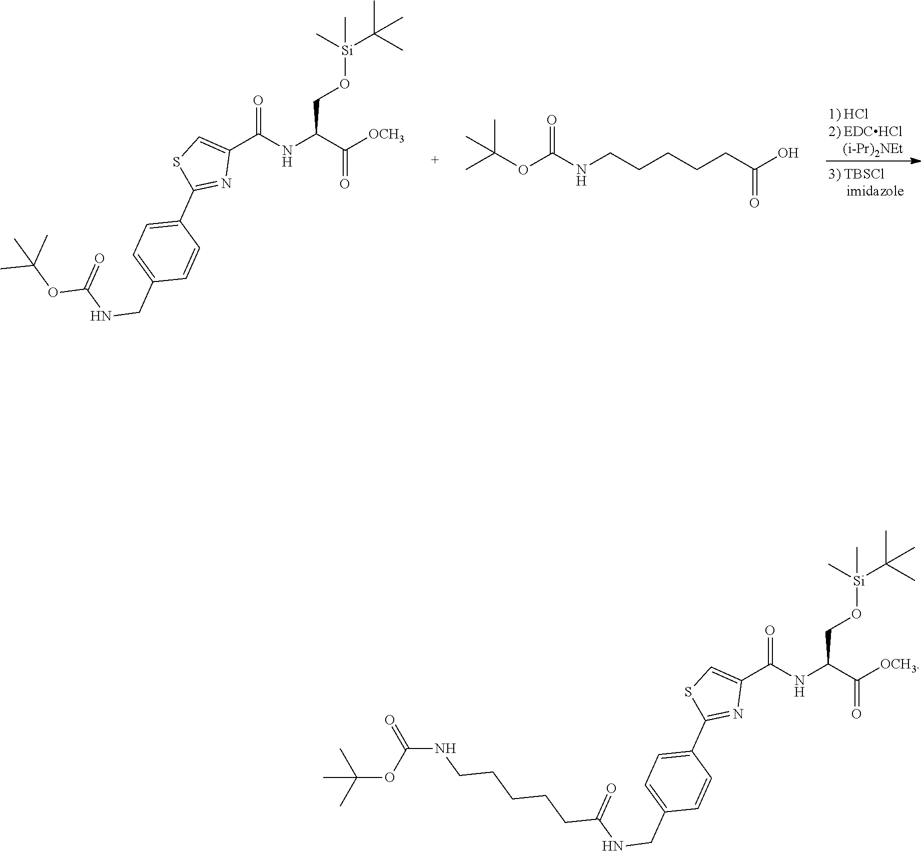 [SMILES: COC(=O)[C@H](CO[Si](C)(C)C(C)(C)C)NC(=O)c1csc(-c2ccc(CNC(=O)OC(C)(C)C)cc2)n1 ; CC(C)(C)OC(=O)NCCCCCC(=O)O ; COC(=O)[C@H](CO[Si](C)(C)C(C)(C)C)NC(=O)c1csc(-c2ccc(CNC(=O)CCCCCNC(=O)OC(C)(C)C)cc2)n1]

Methyl (2-(4-(((tert-butoxycarbonyl)amino)methyl)phenyl)thiazole-4-carbonyl)-L-serinate (Example 35.4, 1.440 g, 3.31 mmole) was dissolved in DCM (6.5 mL). HCl (4M solution in 1,4-dioxane, 3.30 mL, 13.2 mmole) was added and the mixture was stirred at RT for 2 hours, then was concentrated. The residue was suspended in DCM (6.5 mL) and 6-((tert-butoxycarbonyl)amino)hexanoic acid (0.993 g, 4.29 mmole) was added. N,N-Diisopropylethylamine (1.15 mL, 6.60 mmole) was added followed by EDC·HCl (0.822 g, 4.29 mmole) and the mixture was stirred at RT for 18 hours. Water (25 mL) was added and the mixture was extracted with DCM (3×10 mL). The combined organics were dried over magnesium sulfate, filtered and concentrated. The residue was dissolved in DCM (13 mL) and imidazole (0.249 g, 3.66 mmole) and tert-butyldimethylchlorosilane (0.560 g, 3.72 mmole) were added. The mixture was stirred at RT for 2 hours before water (25 mL) was added. The mixture was extracted with DCM (3×10 mL) and the combined organics were dried over magnesium sulfate, filtered and concentrated. The crude residue was purified by silica gel chromatography to provide methyl N-(2-(4-((6-((tert-butoxycarbonyl)amino)hexanamido)methyl)phenyl)thiazole-4-carbonyl)-O-(tert-butyldimethylsilyl)-L-serinate as a colorless oil (1.781 g, 81%). ¹H NMR (400 MHz, CDCl₃): δ 8.21 (d, J=8.7 Hz, 1H), 8.07 (s, 1H), 7.95-7.88 (m, 2H), 7.39-7.31 (m, 2H), 6.01 (s, 1H), 4.89-4.81 (m, 1H), 4.57 (s, 1H), 4.49 (d, J=5.9 Hz, 2H), 4.21 (dd, J=10.1, 2.7 Hz, 1H), 3.95 (dd, J=10.1, 3.4 Hz, 1H), 3.79 (s, 3H), 3.11 (q, J=6.7 Hz, 2H), 2.26 (t, J=7.5 Hz, 2H), 1.75-1.66 (m, 3H), 1.57-1.47 (m, 2H), 1.43 (s, 9H), 1.40-1.32 (m, 2H), 0.92 (s, 9H), 0.08 (s, 3H), 0.06 (s, 3H).

### Methyl O-acetyl-N—(N-(2-(4-((6-((tert-butoxycarbonyl)amino)hexanamido)methyl)phenyl)thiazole-4-carbonyl)-O-(tert-butyldimethylsilyl)-L-seryl)-L-serinate (44.2)

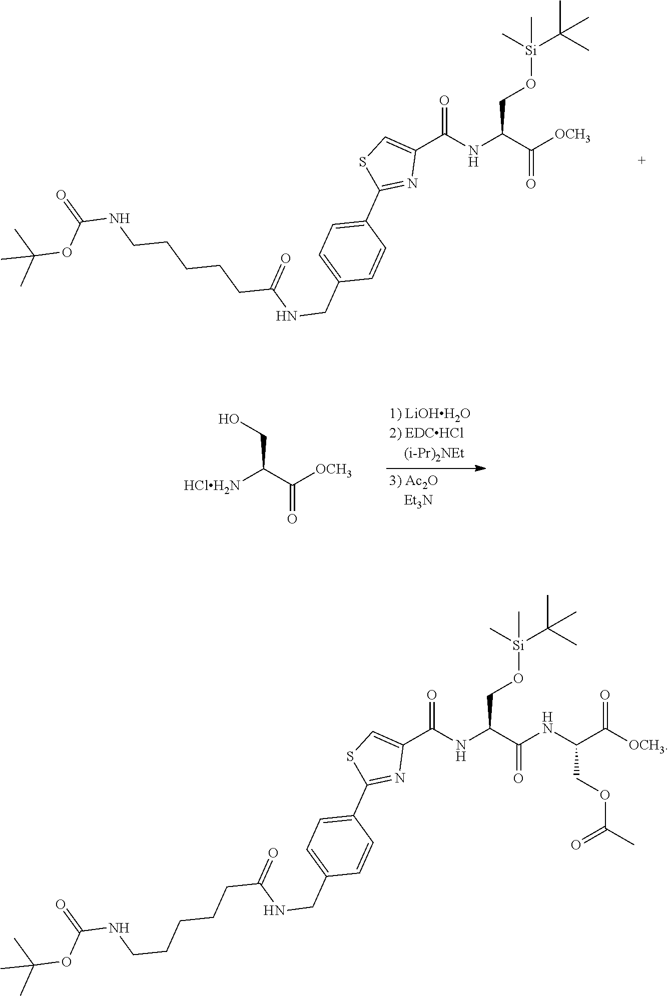

Methyl N-(2-(4-((6-((tert-butoxycarbonyl)amino)hexanamido)methyl)phenyl)thiazole-4-carbonyl)-O-(tert-butyldimethylsilyl)-L-serinate (1.781 g, 2.69 mmole) was dissolved in 4/1/1 THF/methanol/water (5.4 mL). Lithium hydroxide monohydrate (0.233 g, 5.55 mmole) was added and the mixture was stirred at RT for 3 hours and water (25 mL) was added. The mixture was treated with 1N aq. HCl to pH=4 and extracted with ethyl acetate (3×10 mL). The combined organics were dried over magnesium sulfate, filtered and concentrated. The residue was dissolved in DCM (5.4 mL) and L-serine methyl ester hydrochloride (0.538 g, 3.46 mmole) was added. N,N-Diisopropylethylamine (0.940 mL, 5.40 mmole) and EDC·HCl (0.677 g, 3.53 mmole) were added and the mixture was stirred at RT for 20 hours. Water (25 mL) was added and the two layers were separated. The aqueous layer was extracted with DCM (2×10 mL) and the combined organics were dried over magnesium sulfate, filtered and concentrated. The residue was dissolved in DCM (5.4 mL) and triethylamine (0.420 mL, 3.00 mmole) and acetic anhydride (0.280 mL, 2.96 mmole) were added. The mixture was stirred at RT for 20 hours and water (25 mL) was added. The two layers were separated and the aqueous layer was extracted with DCM (2×10 mL). The combined organics were dried over magnesium sulfate, filtered and concentrated. The crude residue was purified by silica gel chromatography to provide methyl O-acetyl-N—(N-(2-(4-((6-((tert-butoxycarbonyl)amino)hexanamido)methyl)phenyl)thiazole-4-carbonyl)-O-(tert-butyldimethylsilyl)-L-seryl)-L-serinate as a colorless oil (1.092 g, 51%). ¹H NMR (400 MHz, CDCl₃): δ 8.26 (d, J=7.1 Hz, 1H), 8.10 (d, J=2.7 Hz, 1H), 7.97-7.89 (m, 2H), 7.48 (d, J=7.7 Hz, 1H), 7.37-7.31 (m, 2H), 6.11-5.90 (m, 1H), 4.90 (dq, J=6.9, 3.5 Hz, 1H), 4.66 (td, J=7.3, 3.7 Hz, 1H), 4.58 (d, J=7.5 Hz, 1H), 4.53-4.47 (m, 3H), 4.44 (td, J=6.2, 4.1 Hz, 1H), 4.34 (dd, J=11.4, 3.6 Hz, 1H), 4.22 (dt, J=9.9, 3.5 Hz, 1H), 3.85-3.80 (m, 1H), 3.79-3.76 (m, 3H), 3.11 (q, J=6.7 Hz, 2H), 2.26 (t, J=7.5 Hz, 3H), 2.02 (d, J=6.8 Hz, 3H), 1.71 (p, J=7.5 Hz, 2H), 1.51 (p, J=7.3 Hz, 2H), 1.43 (s, 9H), 1.40-1.32 (m, 2H), 0.96 (s, 9H), 0.17 (s, 3H), 0.15 (s, 3H).

### Methyl 2-(2-(2-(4-((6-((tert-butoxycarbonyl)amino)hexanamido)methyl)phenyl)thiazole-4-carboxamido)acrylamido)acrylate (44)

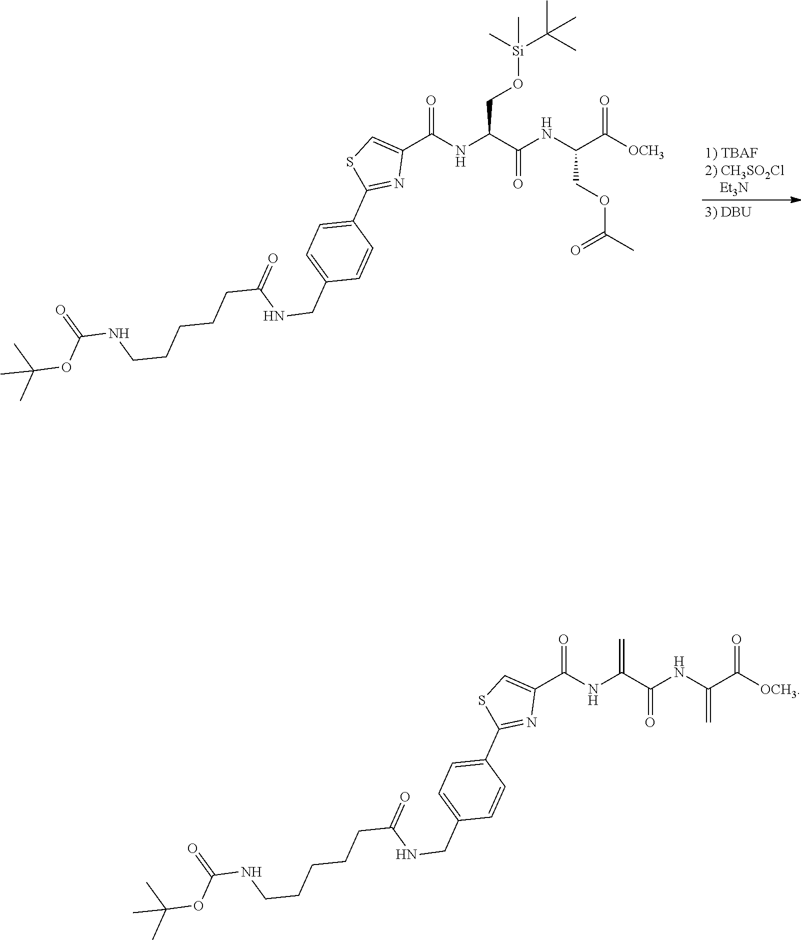 [SMILES: COC(=O)[C@H](COC(C)=O)NC(=O)[C@H](CO[Si](C)(C)C(C)(C)C)NC(=O)c1csc(-c2ccc(CNC(=O)CCCCCNC(=O)OC(C)(C)C)cc2)n1 ; C=C(NC(=O)c1csc(-c2ccc(CNC(=O)CCCCCNC(=O)OC(C)(C)C)cc2)n1)C(=O)NC(=C)C(=O)OC]

Methyl O-acetyl-N—(N-(2-(4-((6-((tert-butoxycarbonyl)amino)hexanamido)methyl)phenyl)thiazole-4-carbonyl)-O-(tert-butyldimethylsilyl)-L-seryl)-L-serinate (1.092 g, 1.38 mmole) was dissolved in THE (2.8 mL). TBAF (1M solution in THF, 3.50 mL, 3.50 mmole) was added and the solution was stirred at RT for 2 hours. Water (25 mL) was added and the mixture was extracted with ethyl acetate (3×10 mL). The combined organics were dried over magnesium sulfate, filtered and concentrated. The residue was dissolved in DCM (5.6 mL) and cooled to 0° C. Triethylamine (0.290 mL, 2.07 mmole) and methanesulfonyl chloride (0.160 mL, 2.07 mmole) were added and the mixture was stirred at 0° for 60 minutes. Water (25 mL) was added and the mixture was extracted with DCM (3×10 mL). The combined organics were dried over magnesium sulfate, filtered and concentrated. The residue was dissolved in THE (5.6 mL) and cooled to 0° C. DBU (0.310 mL, 2.07 mmole) was added and the mixture was stirred at 0° for 60 minutes. Water (25 mL) was added and the mixture was extracted with ethyl acetate (3×10 mL). The combined organics were dried over magnesium sulfate, filtered and concentrated. The crude residue was purified by silica gel chromatography to provide methyl 2-(2-(2-(4-((6-((tert-butoxycarbonyl)amino)hexanamido)methyl)phenyl)thiazole-4-carboxamido)acrylamido)acrylate as a white solid (0.255 g, 31%). ¹H NMR (400 MHz, CDCl₃): δ 10.01 (s, 1H), 8.55 (s, 1H), 8.14 (s, 1H), 8.03-7.91 (m, 2H), 7.37 (d, J=8.2 Hz, 2H), 6.78 (d, J=2.2 Hz, 1H), 6.71 (s, 1H), 6.09-6.00 (m, 1H), 5.86 (s, 1H), 5.50 (t, J=1.9 Hz, 1H), 4.55 (s, 1H), 4.50 (d, J=5.8 Hz, 2H), 3.91 (s, 3H), 3.12 (q, J=6.7 Hz, 2H), 2.26 (t, J=7.5 Hz, 2H), 1.71 (p, J=7.6 Hz, 2H), 1.51 (q, J=7.3 Hz, 2H), 1.43 (s, 9H), 1.40-1.32 (m, 2H).

### Compound 45: Methyl 2-(2-(2-(4-(((((tetrahydro-2H-pyran-4-yl)methoxy)carbonyl)amino)methyl)phenyl)thiazole-4-carboxamido)acrylamido)acrylate

### Ethyl 2-(4-(aminomethyl)phenyl)thiazole-4-carboxylate hydrobromide (45.1)

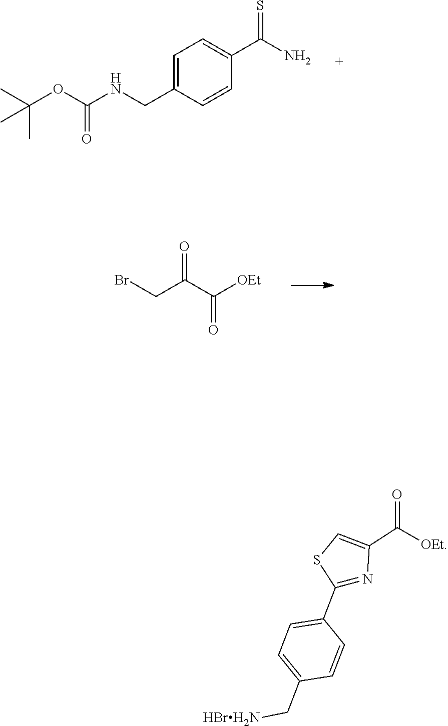

Tert-butyl (4-carbamothioylbenzyl)carbamate (Example 35.2, 7.744 g, 29.1 mmole) was dissolved in ethanol (60 mL) and ethyl bromopyruvate (4.40 mL, 35.1 mmole) was added. The mixture was stirred at 80° C. for 3 hours and was cooled to RT. The precipitated solid was collected by filtration, washed with a small amount of ethanol and allowed to air dry to provide ethyl 2-(4-(aminomethyl)phenyl)thiazole-4-carboxylate hydrobromide as a pale yellow solid (7.650 g, 77%). ¹H NMR (400 MHz, d₆-DMSO): δ 8.61 (s, 1H), 8.44-8.24 (m, 3H), 8.11-8.01 (m, 2H), 7.69-7.61 (m, 2H), 4.35 (q, J=7.1 Hz, 2H), 4.17-4.09 (m, 2H), 1.34 (t, J=7.1 Hz, 3H).

### Ethyl 2-(4-(((((tetrahydro-2H-pyran-4-yl)methoxy)carbonyl)amino)methyl)phenyl)thiazole-4-carboxylate (45.2)

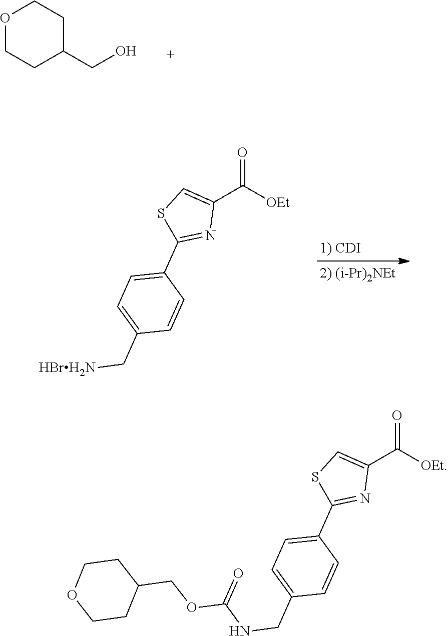

(Tetrahydro-2H-pyran-4-yl)methanol (0.279 g, 2.40 mmole) was dissolved in THE (4.2 mL) and CDI (0.408 g, 2.52 mmole) was added slowly. The mixture was stirred at RT for 18 hours. Water (25 mL) was added and the mixture was extracted with ethyl acetate (3×10 mL). The combined organics were dried over magnesium sulfate, filtered and concentrated. The residue was dissolved in DMF (4.2 mL) and ethyl 2-(4-(aminomethyl)phenyl)thiazole-4-carboxylate hydrobromide (0.753 g, 2.19 mmole) and N,N-diisopropylethylamine (0.460 mL, 2.64 mmole) were added. The mixture was warmed to 50° C. for 18 hours, then was cooled to RT and diluted with ethyl acetate (25 mL). The mixture was washed sequentially with water (3×25 mL) and sat. aq. sodium chloride (1×25 mL), then dried over magnesium sulfate, filtered and concentrated. The crude residue was purified by silica gel chromatography to provide ethyl 2-(4-(((((tetrahydro-2H-pyran-4-yl)methoxy)carbonyl)amino)methyl)phenyl)thiazole-4-carboxylate as an orange solid (0.613 g, 69%). ¹H NMR (400 MHz, CDCl₃): δ 8.15 (s, 1H), 8.03-7.94 (m, 2H), 7.37 (d, J=7.9 Hz, 2H), 5.09 (s, 1H), 4.50-4.35 (m, 4H), 3.97 (t, J=5.5 Hz, 4H), 3.39 (t, J=11.1 Hz, 2H), 1.61 (s, 2H), 1.43 (t, J=7.1 Hz, 3H), 1.40-1.29 (m, 2H).

### Methyl O-(tert-butyldimethylsilyl)-N-(2-(4-(((((tetrahydro-2H-pyran-4-yl)methoxy)carbonyl)amino)methyl)phenyl)thiazole-4-carbonyl)-L-serinate (45.3)

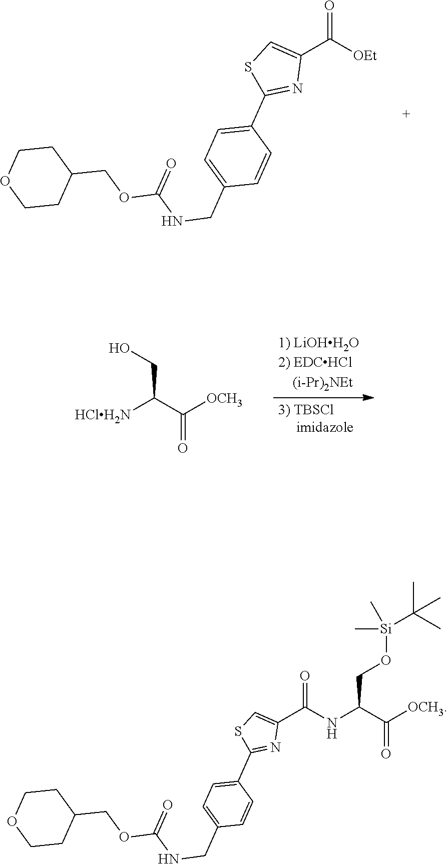

Ethyl 2-(4-(((((tetrahydro-2H-pyran-4-yl)methoxy)carbonyl)amino)methyl)phenyl)thiazole-4-carboxylate (0.613 g, 1.52 mmole) was dissolved in 4/1/1 THF/methanol/water (3 mL). Lithium hydroxide monohydrate (0.097 g, 2.31 mmole) was added and the mixture was stirred at RT for 3 hours. Water (25 mL) was added and the solution was treated with 1N aq. HCl to pH=4. The mixture was extracted with ethyl acetate (3×10 mL). The combined organics were dried over magnesium sulfate, filtered and concentrated. The residue was suspended in DCM (6 mL). L-Serine methyl ester hydrochloride (0.307 g, 1.97 mmole) was added followed by N,N-diisopropylethylamine (0.530 mL, 3.04 mmole) and EDC·HCl (0.385 g, 2.01 mmole). The mixture was stirred at RT for 2 days. Water (25 mL) was added and the mixture was extracted with DCM (3×10 mL). The combined organics were dried over magnesium sulfate, filtered and concentrated. The residue was dissolved in DCM (3 mL) and imidazole (0.120 g, 1.76 mmole) and tert-butyldimethylchlorosilane (0.250 g, 1.66 mmole) were added. The mixture was stirred at RT for 3 hours and water (25 mL) was added. The mixture was extracted with DCM (3×10 mL) and the combined organics were dried over magnesium sulfate, filtered and concentrated. The crude residue was purified by silica gel chromatography to provide methyl O-(tert-butyldimethylsilyl)-N-(2-(4-(((((tetrahydro-2H-pyran-4-yl)methoxy)carbonyl)amino)methyl)phenyl)thiazole-4-carbonyl)-L-serinate as a thick, colorless oil (0.329 g, 37%). ¹H NMR (400 MHz, CDCl₃): δ 8.21 (d, J=8.7 Hz, 1H), 8.10 (s, 1H), 7.99-7.88 (m, 2H), 7.38 (d, J=7.8 Hz, 2H), 5.09 (s, 1H), 4.86 (dt, J=8.7, 3.0 Hz, 1H), 4.43 (d, J=6.1 Hz, 2H), 4.21 (dd, J=10.0, 2.6 Hz, 1H), 4.04-3.92 (m, 5H), 3.79 (s, 3H), 3.39 (t, J=11.8 Hz, 2H), 1.90 (q, J=7.9, 5.7 Hz, 1H), 1.61 (s, 3H), 1.39 (tt, J=14.2, 6.9 Hz, 2H), 0.92 (s, 9H), 0.07 (s, 3H), 0.06 (s, 3H).

### Methyl O-acetyl-N-(0-(tert-butyldimethylsilyl)-N-(2-(4-(((((tetrahydro-2H-pyran-4-yl)methoxy)carbonyl)amino)methyl)phenyl)thiazole-4-carbonyl)-L-seryl)-L-serinate (45.4)

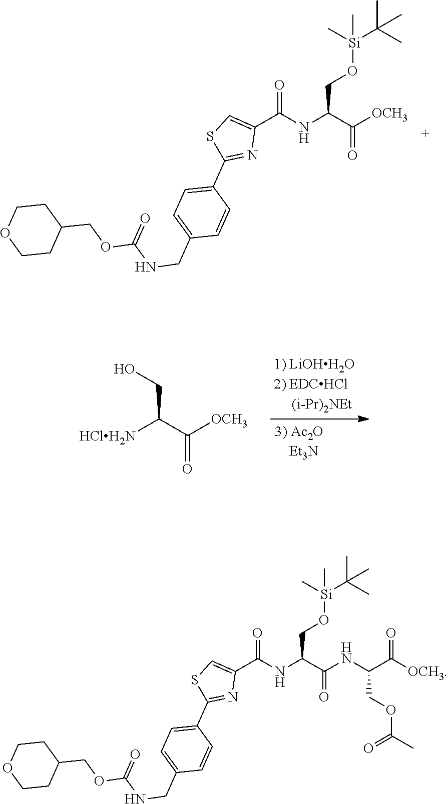

Methyl O-(tert-butyldimethylsilyl)-N-(2-(4-(((((tetrahydro-2H-pyran-4-yl)methoxy)carbonyl)amino)methyl)phenyl)thiazole-4-carbonyl)-L-serinate (0.329 g, 0.556 mmole) was dissolved in 4/1/1 THF/methanol/water (2.2 mL). Lithium hydroxide monohydrate (0.039 g, 0.929 mmole) was added and the mixture was stirred at RT for 3 hours. Water (25 mL) was added and the solution was treated with 1N aq. HCl to pH=4. The mixture was extracted with ethyl acetate (6×10 mL) and the combined organics were dried over magnesium sulfate, filtered and concentrated. The residue was suspended in DCM (2.2 mL). L-Serine methyl ester hydrochloride (0.113 g, 0.726 mmole) was added followed by N,N-diisopropylethylamine (0.195 mL, 1.12 mmole) and EDC·HCl (0.146 g, 0.762 mmole). The mixture was stirred at RT for 18 hours and water (25 mL) was added. The mixture was extracted with DCM (3×10 mL) and the combined organics were dried over magnesium sulfate, filtered and concentrated. The residue was dissolved in DCM (2.2 mL) and triethylamine (0.086 mL, 0.614 mmole) and acetic anhydride (0.058 mL, 0.614 mmole) were added. The solution was stirred at RT for 2 hours and water (25 mL) was added. The mixture was extracted with DCM (3×10 mL) and the combined organics were dried over magnesium sulfate, filtered and concentrated. The crude residue was purified by silica gel chromatography to provide methyl O-acetyl-N—(O-(tert-butyldimethylsilyl)-N-(2-(4-(((((tetrahydro-2H-pyran-4-yl)methoxy)carbonyl)amino)methyl)phenyl)thiazole-4-carbonyl)-L-seryl)-L-serinate as a thick oil (0.223 g, 56%). ¹H NMR (400 MHz, CDCl₃): δ 8.26 (d, J=7.0 Hz, 1H), 8.11 (d, J=2.3 Hz, 1H), 7.95 (dd, J=8.3, 1.8 Hz, 2H), 7.47 (d, J=7.8 Hz, 1H), 7.37 (d, J=8.0 Hz, 2H), 5.06 (s, 1H), 4.90 (tt, J=7.4, 3.8 Hz, 1H), 4.66 (td, J=7.3, 3.7 Hz, 1H), 4.54-4.45 (m, 1H), 4.45-4.38 (m, 2H), 4.34 (dd, J=11.4, 3.6 Hz, 1H), 4.23 (ddd, J=9.8, 3.8, 2.7 Hz, 1H), 3.99 (d, J=6.5 Hz, 4H), 3.82 (dd, J=10.6, 3.1 Hz, 1H), 3.77 (s, 3H), 3.39 (t, J=11.7 Hz, 2H), 2.01 (s, 2H), 1.98-1.83 (m, 1H), 1.63 (d, J=13.3 Hz, 2H), 1.39 (td, J=14.7, 13.4, 6.6 Hz, 2H), 0.96 (s, 9H), 0.17 (s, 3H), 0.15 (s, 3H).

### Methyl 2-(2-(2-(4-(((((tetrahydro-2H-pyran-4-yl)methoxy)carbonyl)amino)methyl)phenyl)thiazole-4-carboxamido)acrylamido)acrylate (45)

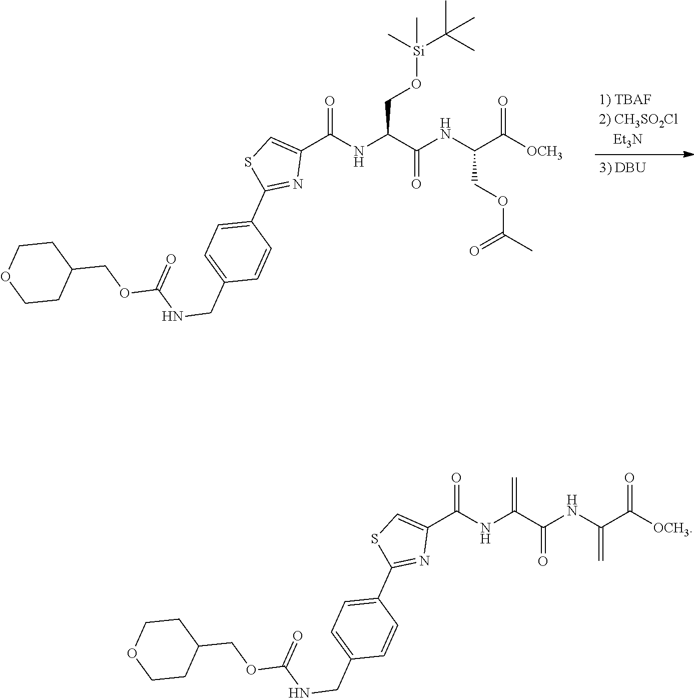 [SMILES: COC(=O)[C@H](COC(C)=O)NC(=O)[C@H](CO[Si](C)(C)C(C)(C)C)NC(=O)c1csc(-c2ccc(CNC(=O)OCC3CCOCC3)cc2)n1 ; C=C(NC(=O)c1csc(-c2ccc(CNC(=O)OCC3CCOCC3)cc2)n1)C(=O)NC(=C)C(=O)OC]

Methyl O-acetyl-N—(O-(tert-butyldimethylsilyl)-N-(2-(4-(((((tetrahydro-2H-pyran-4-yl)methoxy)carbonyl)amino)methyl)phenyl)thiazole-4-carbonyl)-L-seryl)-L-serinate (0.223 g, 0.309 mmole) was dissolved in THE (2 mL). TBAF (1M solution in THF, 0.780 mL, 0.780 mmole) was added and the solution was stirred at RT for 2 hours. Water (25 mL) and sat. aq. sodium chloride (10 mL) were added and the mixture was extracted with ethyl acetate (3×10 mL). The combined organics were dried over magnesium sulfate, filtered and concentrated. The residue was dissolved in DCM (2 mL) and cooled to 0° C. Triethylamine (0.065 mL, 0.464 mmole) and methanesulfonyl chloride (0.036 mL, 0.465 mmole) were added and the mixture was stirred at 0° for 60 minutes and water (25 mL) was added. The mixture was extracted with DCM (3×10 mL) and the combined organics were dried over magnesium sulfate, filtered and concentrated. The residue was dissolved in THE (2 mL) and cooled to 0° C. DBU (0.070 mL, 0.468 mmole) was added and the mixture was stirred at 0° for 45 minutes. Water (25 mL) was added and the mixture was extracted with DCM (3×10 mL). The combined organics were dried over magnesium sulfate, filtered and concentrated. The crude residue was purified by silica gel chromatography to provide methyl 2-(2-(2-(4-(((((tetrahydro-2H-pyran-4-yl)methoxy)carbonyl)amino)methyl)phenyl)thiazole-4-carboxamido)acrylamido)acrylate as a white solid (0.086 g, 53%). ¹H NMR (400 MHz, CDCl₃): δ 10.02 (s, 1H), 8.55 (s, 1H), 8.14 (s, 1H), 8.02-7.94 (m, 2H), 7.39 (d, J=7.9 Hz, 2H), 6.78 (d, J=2.2 Hz, 1H), 6.74-6.66 (m, 1H), 6.04 (d, J=1.2 Hz, 1H), 5.50 (t, J=1.9 Hz, 1H), 5.08 (s, 1H), 4.43 (d, J=6.1 Hz, 2H), 3.99 (d, J=6.5 Hz, 4H), 3.91 (s, 3H), 3.46-3.34 (m, 2H), 2.00-1.84 (m, 1H), 1.68-1.57 (m, 2H), 1.38 (qd, J=11.9, 4.0 Hz, 2H).

### Compound 46: Methyl 2-(2-(2-(4-(tetrahydro-2H-pyran-4-carboxamido)piperidin-1-yl)thiazole-4-carboxamido)acrylamido)acrylate

### Ethyl 2-(4-((tert-butoxycarbonyl)amino)piperidin-1-yl)thiazole-4-carboxylate (46.1)

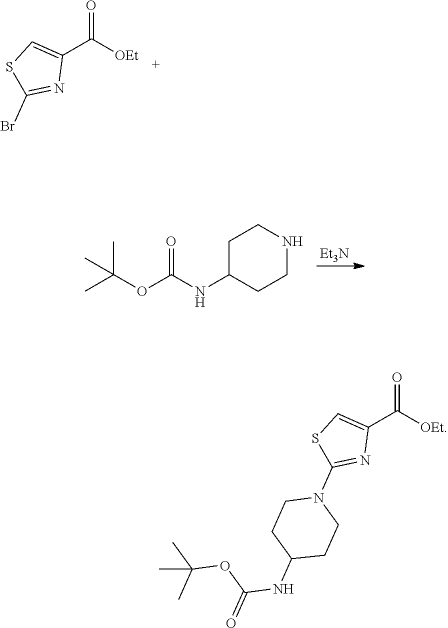 [SMILES: "Et3N" ; CCOC(=O)c1csc(Br)n1 ; CC(C)(C)OC(=O)NC1CCNCC1 ; CCOC(=O)c1csc(N2CCC(NC(=O)OC(C)(C)C)CC2)n1]

Ethyl 2-bromothiazole-4-carboxylate (3.021 g, 12.8 mmole) and tert-butyl piperidin-4-ylcarbamate (3.065 g, 15.3 mmole) were dissolved in DMA (13 mL). Triethylamine (2.15 mL, 15.3 mmole) was added and the mixture was heated to 80° C. for 18 hours, then was cooled to RT and diluted with ethyl acetate (50 mL). The mixture was washed with water (3×25 mL) and the organics were dried over magnesium sulfate, filtered, and concentrated. The crude residue was purified by silica gel chromatography to provide ethyl 2-(4-((tert-butoxycarbonyl)amino)piperidin-1-yl)thiazole-4-carboxylate as a white solid (4.073 g, 90%). ¹H NMR (400 MHz, CDCl₃): δ 7.42 (s, 1H), 4.48 (d, J=7.9 Hz, 1H), 4.34 (q, J=7.1 Hz, 2H), 4.03-3.94 (m, 2H), 3.67 (s, 1H), 3.14 (ddd, J=13.3, 11.6, 3.0 Hz, 2H), 2.09-1.98 (m, 2H), 1.55-1.46 (m, 2H), 1.44 (s, 9H), 1.36 (t, J=7.1 Hz, 3H).

### Methyl (2-(4-((tert-butoxycarbonyl)amino)piperidin-1-yl)thiazole-4-carbonyl)-L-serinate (46.2)

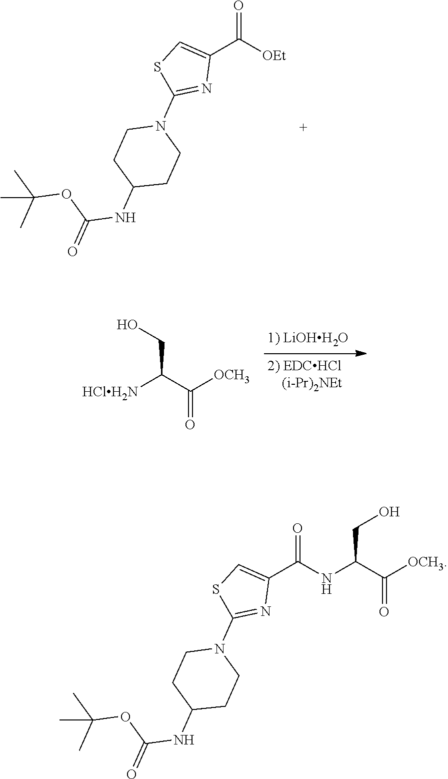

Ethyl 2-(4-((tert-butoxycarbonyl)amino)piperidin-1-yl)thiazole-4-carboxylate (3.371 g, 10.5 mmole) was dissolved in 4/1/1 THF/methanol/water (21 mL). Lithium hydroxide monohydrate (0.667 g, 15.9 mmole) was added and the mixture was stirred at RT for 2 hours. Water (50 mL) was added and the solution was treated with 1N aq. HCl to pH=4. The mixture was extracted with ethyl acetate (3×15 mL) and the combined organics were dried over magnesium sulfate, filtered and concentrated. The residue was suspended in DCM (21 mL) and L-serine methyl ester hydrochloride (2.119 g, 13.6 mmole) was added. N,N-Diisopropylethylamine (3.70 mL, 21.2 mmole) and EDC·HCl (2.629 g, 13.7 mmole) were added and the mixture was stirred at RT for 18 hours. Water (50 mL) was added and the two layers were separated. The aqueous layer was extracted with DCM (2×15 mL) and the combined organics were dried over magnesium sulfate, filtered and concentrated. The crude residue was purified by silica gel chromatography to provide methyl (2-(4-((tert-butoxycarbonyl)amino)piperidin-1-yl)thiazole-4-carbonyl)-L-serinate as a white solid (2.566 g, 57%). ¹H NMR (400 MHz, CDCl₃): δ 7.98 (d, J=7.4 Hz, 1H), 7.39 (s, 1H), 4.79 (dt, J=7.6, 3.9 Hz, 1H), 4.53 (s, 1H), 4.04 (ddd, J=6.2, 3.8, 1.0 Hz, 2H), 3.95 (dq, J=12.7, 3.6, 2.8 Hz, 2H), 3.82 (s, 3H), 3.66 (d, J=25.2 Hz, 1H), 3.21-3.08 (m, 2H), 2.87 (t, J=6.1 Hz, 1H), 2.11-1.99 (m, 2H), 1.56-1.48 (m, 2H), 1.46 (s, 9H).

### Methyl O-(tert-butyldimethylsilyl)-N-(2-(4-(tetrahydro-2H-pyran-4-carboxamido)piperidin-1-yl)thiazole-4-carbonyl)-L-serinate (46.3)

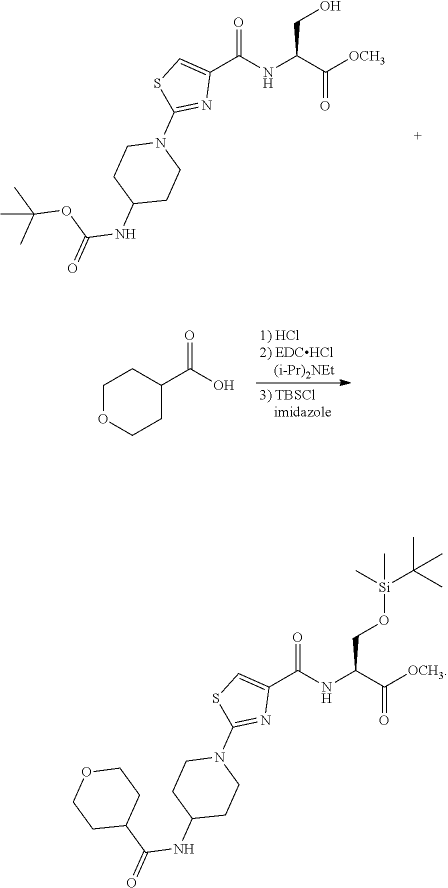 [SMILES: COC(=O)[C@H](CO)NC(=O)c1csc(N2CCC(NC(=O)OC(C)(C)C)CC2)n1 ; O=C(O)C1CCOCC1 ; COC(=O)[C@H](CO[Si](C)(C)C(C)(C)C)NC(=O)c1csc(N2CCC(NC(=O)C3CCOCC3)CC2)n1]

Methyl (2-(4-((tert-butoxycarbonyl)amino)piperidin-1-yl)thiazole-4-carbonyl)-L-serinate (0.510 g, 1.19 mmole) was dissolved in DCM (2.3 mL). HCl (4M solution in 1,4-dioxane, 1.20 mL, 4.80 mmole) was added and the mixture was stirred at RT for 4 hours, then was concentrated. The residue was suspended in DCM (2.3 mL) and tetrahydro-2H-pyran-4-carboxylic acid (0.195 g, 1.51 mmole) was added. N,N-Diisopropylethylamine (0.410 mL, 2.35 mmole) and EDC·HCl (0.294 g, 1.53 mmole) were added and the mixture was stirred at RT for 18 hours. Water (25 mL) was added and the mixture was extracted with DCM (3×10 mL). The combined organics were dried over magnesium sulfate, filtered and concentrated. The residue was dissolved in DCM (3 mL) and imidazole (0.090 g, 1.32 mmole) and tert-butyldimethylchlorosilane (0.195 g, 1.29 mmole) were added. The mixture was stirred at RT for 3 hours and water (25 mL) was added. The mixture was extracted with DCM (3×10 mL) and the combined organics were dried over magnesium sulfate, filtered and concentrated. The crude residue was purified by silica gel chromatography to provide methyl O-(tert-butyldimethylsilyl)-N-(2-(4-(tetrahydro-2H-pyran-4-carboxamido)piperidin-1-yl)thiazole-4-carbonyl)-L-serinate as a white solid (0.419 g, 63%). ¹H NMR (400 MHz, CDCl₃): δ 7.95 (d, J=8.8 Hz, 1H), 7.38 (s, 1H), 5.39 (d, J=7.9 Hz, 1H), 4.83-4.74 (m, 1H), 4.18-4.13 (m, 1H), 4.08-3.98 (m, 4H), 3.98-3.90 (m, 1H), 3.88 (dd, J=10.0, 3.4 Hz, 1H), 3.76 (s, 3H), 3.41 (td, J=11.4, 2.9 Hz, 2H), 3.22-3.08 (m, 2H), 2.32 (tt, J=11.0, 4.5 Hz, 1H), 2.04-1.98 (m, 2H), 1.87-1.70 (m, 4H), 1.50 (ddt, J=15.3, 12.0, 7.8 Hz, 2H), 0.88 (s, 9H), 0.05 (s, 3H), 0.03 (s, 3H).

### Methyl O-acetyl-N—(O-(tert-butyldimethylsilyl)-N-(2-(4-(tetrahydro-2H-pyran-4-carboxamido)piperidin-1-yl)thiazole-4-carbonyl)-L-seryl)-L-serinate (46.4)

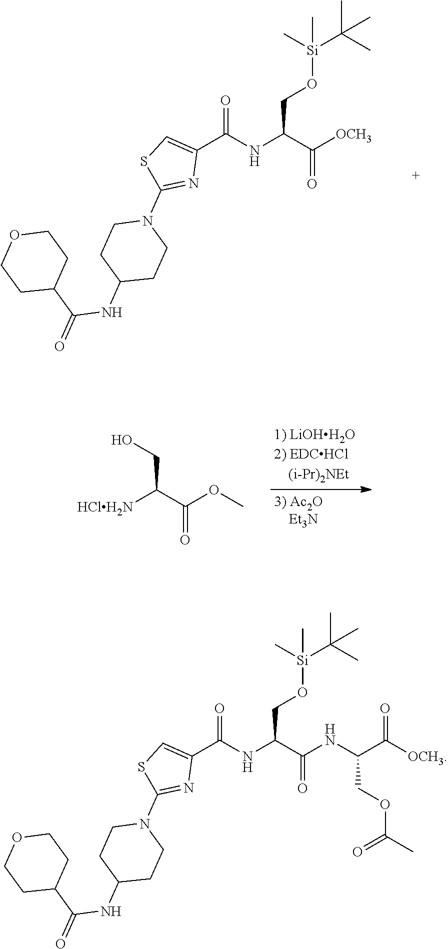

Methyl O-(tert-butyldimethylsilyl)-N-(2-(4-(tetrahydro-2H-pyran-4-carboxamido)piperidin-1I-yl)thiazole-4-carbonyl)-L-serinate (0.419 g, 0.755 mmole) was dissolved in 4/1/1 THF/methanol/water (3.2 mL). Lithium hydroxide monohydrate (0.050 g, 1.19 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was suspended in DCM (3.2 mL). L-Serine methyl ester hydrochloride (0.152 g, 0.977 mmole) was added followed by N,N-diisopropylethylamine (0.265 mL, 1.52 mmole) and EDC·HC (0.185 g, 0.965 mmole). The mixture was stirred at RT for 18 hours and water (25 mL) was added. The mixture was extracted with DCM (3×10 mL) and the combined organics were dried over magnesium sulfate, filtered and concentrated. The residue was dissolved in DCM (3.2 mL) and triethylamine (0.120 mL, 0.856 mmole) and acetic anhydride (0.080 mL, 0.846 mmole) were added. The solution was stirred at RT for 2 hours and water (25 mL) was added. The mixture was extracted with DCM (3×10 mL) and the combined organics were dried over magnesium sulfate, filtered and concentrated. The crude residue was purified by silica gel chromatography to provide methyl O-acetyl-N-(0-(tert-butyldimethylsilyl)-N-(2-(4-(tetrahydro-2H-pyran-4-carboxamido)piperidin-1-yl)thiazole-4-carbonyl)-L-seryl)-L-serinate as a colorless oil (0.212 g, 41%). ¹H NMR (400 MHz, CDCl₃): δ 7.98 (d, J=7.1 Hz, 1H), 7.44 (d, J=7.8 Hz, 1H), 7.40 (s, 1H), 5.38 (d, J=7.9 Hz, 1H), 4.87 (dt, J=7.6, 3.8 Hz, 1H), 4.58 (td, J=7.2, 3.7 Hz, 1H), 4.51-4.43 (m, 1H), 4.32 (dd, J=11.4, 3.7 Hz, 1H), 4.18 (dd, J=9.8, 3.7 Hz, 1H), 4.02 (ddd, J=11.5, 4.2, 2.5 Hz, 4H), 3.94 (dd, J=9.9, 6.4 Hz, 1H), 3.77 (s, 3H), 3.75-3.68 (m, 1H), 3.41 (td, J=11.4, 2.9 Hz, 2H), 3.21-3.05 (m, 2H), 2.32 (tt, J=11.0, 4.5 Hz, 1H), 2.02 (d, J=3.4 Hz, 4H), 1.84-1.71 (m, 4H), 1.50 (qq, J=11.6, 5.2 Hz, 2H), 0.92 (s, 9H), 0.14 (s, 3H), 0.13 (s, 3H).

### Methyl 2-(2-(2-(4-(tetrahydro-2H-pyran-4-carboxamido)piperidin-1-yl)thiazole-4-carboxamido)acrylamido)acrylate (46)

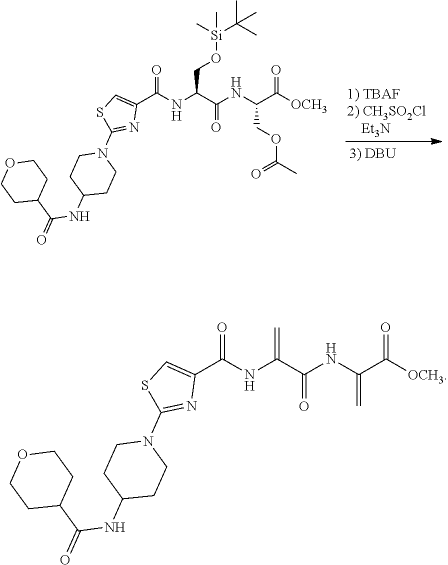 [SMILES: COC(=O)[C@H](COC(C)=O)NC(=O)[C@H](CO[Si](C)(C)C(C)(C)C)NC(=O)c1csc(N2CCC(NC(=O)C3CCOCC3)CC2)n1 ; C=C(NC(=O)c1csc(N2CCC(NC(=O)C3CCOCC3)CC2)n1)C(=O)NC(=C)C(=O)OC]

Methyl O-acetyl-N-(0-(tert-butyldimethylsilyl)-N-(2-(4-(tetrahydro-2H-pyran-4-carboxamido)piperidin-1l-yl)thiazole-4-carbonyl)-L-seryl)-L-serinate (0.212 g, 0.310 mmole) was dissolved in THF (2 mL). TBAF (1M solution in THF, 0.780 mL, 0.780 mmole) was added and the solution was stirred at RT for 2 hours. Water (25 mL) and sat. aq. sodium chloride (10 mL) were added and the mixture was extracted with ethyl acetate (3×10 mL). The combined organics were dried over magnesium sulfate, filtered and concentrated. The residue was dissolved in DCM (2 mL) and cooled to 0° C. Triethylamine (0.066 mL, 0.471 mmole) and methanesulfonyl chloride (0.036 mL, 0.465 mmole) were added and the mixture was stirred at 0° for 60 minutes and water (25 mL) was added. The mixture was extracted with DCM (3×10 mL) and the combined organics were dried over magnesium sulfate, filtered and concentrated. The residue was dissolved in THF (2 mL) and cooled to 0° C. DBU (0.070 mL, 0.468 mmole) was added and the mixture was stirred at 0° for 45 minutes. Water (25 mL) was added and the mixture was extracted with DCM (3×10 mL). The combined organics were dried over magnesium sulfate, filtered and concentrated. The crude residue was purified by silica gel chromatography to provide methyl 2-(2-(2-(4-(tetrahydro-2H-pyran-4-carboxamido)piperidin-1-yl)thiazole-4-carboxamido)acrylamido)acrylate as a white solid (0.043 g, 28%). ¹H NMR (400 MHz, CDCl₃): δ 9.71 (s, 1H), 8.51 (s, 1H), 7.43 (s, 1H), 6.72 (d, J=2.1 Hz, 1H), 6.65 (s, 1H), 6.01 (d, J=1.3 Hz, 1H), 5.44 (t, J=1.9 Hz, 1H), 5.37 (d, J=7.8 Hz, 1H), 4.08-3.96 (m, 5H), 3.89 (s, 3H), 3.41 (td, J=11.4, 2.9 Hz, 2H), 3.17 (ddd, J=13.3, 11.7, 2.9 Hz, 2H), 2.32 (tt, J=11.0, 4.5 Hz, 1H), 2.10-2.02 (m, 2H), 1.88-1.70 (m, 4H), 1.57-1.45 (m, 2H).

### Compound 47: Methyl 2-(2-(2-(4-(2-(2-methoxyethoxy)acetyl)piperazin-1-yl)thiazole-4-carboxamido)acrylamido)acrylate

### Ethyl 2-(4-(tert-butoxycarbonyl)piperazin-1-yl)thiazole-4-carboxylate (47.1)

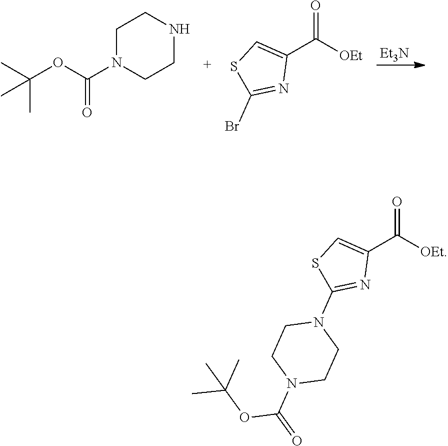 [SMILES: "Et3N" ; CC(C)(C)OC(=O)N1CCNCC1 ; CCOC(=O)c1csc(Br)n1 ; CCOC(=O)c1csc(N2CCN(C(=O)OC(C)(C)C)CC2)n1]

Tert-butyl piperazine-1-carboxylate (1.440 g, 7.73 mmole) was dissolved in DMA (8 mL). Ethyl 2-bromothiazole-4-carboxylate (1.998 g, 8.46 mmole) was added followed by triethylamine (1.30 mL, 9.28 mmole) and the mixture was heated to 80° C. for 18 hours, then was cooled to RT and diluted with ethyl acetate (50 mL). The mixture was washed sequentially with water (3×25 mL) and sat. aq. sodium chloride (1×25 mL). The organics were dried over magnesium sulfate, filtered and concentrated. The crude residue was purified by silica gel chromatography to provide ethyl 2-(4-(tert-butoxycarbonyl)piperazin-1-yl)thiazole-4-carboxylate as a white solid (1.877 g, 71%). ¹H NMR (400 MHz, CDCl₃): δ 7.47 (s, 1H), 4.36 (q, J=7.1 Hz, 2H), 3.61-3.47 (m, 8H), 1.48 (s, 9H), 1.37 (t, J=7.1 Hz, 3H)

### Tert-butyl (S)-4-(4-((3-hydroxy-1-methoxy-1-oxopropan-2-yl)carbamoyl)thiazol-2-yl)piperazine-1-carboxylate (47.2)

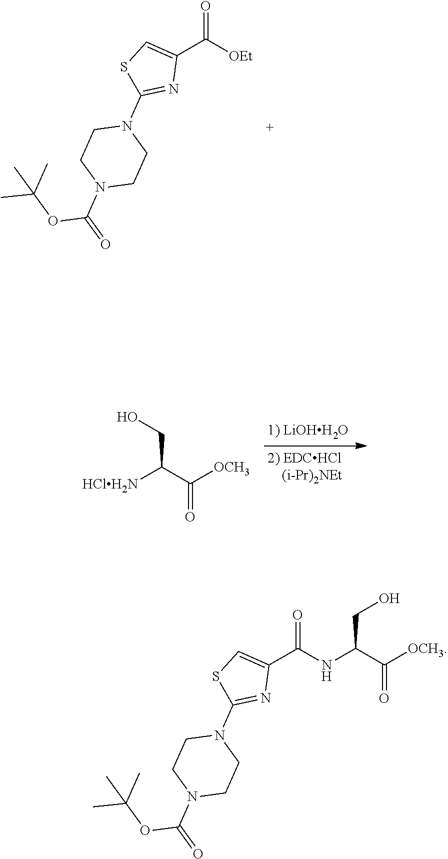

Ethyl 2-(4-(tert-butoxycarbonyl)piperazin-1-yl)thiazole-4-carboxylate (1.877 g, 5.50 mmole) was dissolved in 4/1/1 THF/methanol/water (11 mL). Lithium hydroxide monohydrate (0.344 g, 8.20 mmole) was added and the mixture was stirred at RT for 3 hours. Water (25 mL) was added and the solution was treated with 1N aq. HCl to pH=4. The mixture was extracted with ethyl acetate (6×10 mL) and the combined organics were dried over magnesium sulfate, filtered and concentrated. The residue was suspended in DCM (11 mL). L-Serine methyl ester hydrochloride (1.108 g, 7.12 mmole) was added followed by N,N-diisopropylethylamine (1.90 mL, 10.9 mmole) and EDC·HCl (1.372 g, 7.16 mmole). The mixture was stirred at RT for 18 hours and water (50 mL) was added. The two layers were separated and the aqueous layer was extracted with DCM (2×15 mL). The combined organics were dried over magnesium sulfate, filtered and concentrated. The crude residue was purified by silica gel chromatography to provide tert-butyl (S)-4-(4-((3-hydroxy-1-methoxy-1-oxopropan-2-yl)carbamoyl)thiazol-2-yl)piperazine-1-carboxylate as a white solid (1.229 g, 54%). ¹H NMR (400 MHz, CDCl₃): δ 7.99 (d, J=7.4 Hz, 1H), 7.43 (s, 1H), 4.80 (dt, J=7.6, 3.9 Hz, 1H), 4.09-4.01 (m, 2H), 3.82 (s, 3H), 3.57 (dd, J=6.6, 3.7 Hz, 4H), 3.49 (dd, J=6.2, 3.6 Hz, 4H), 2.82 (t, J=6.0 Hz, 1H), 1.49 (s, 9H).

### Methyl O-(tert-butyldimethylsilyl)-N-(2-(4-(2-(2-methoxyethoxy)acetyl)piperazin-1-yl)thiazole-4-carbonyl)-L-serinate (47.3)

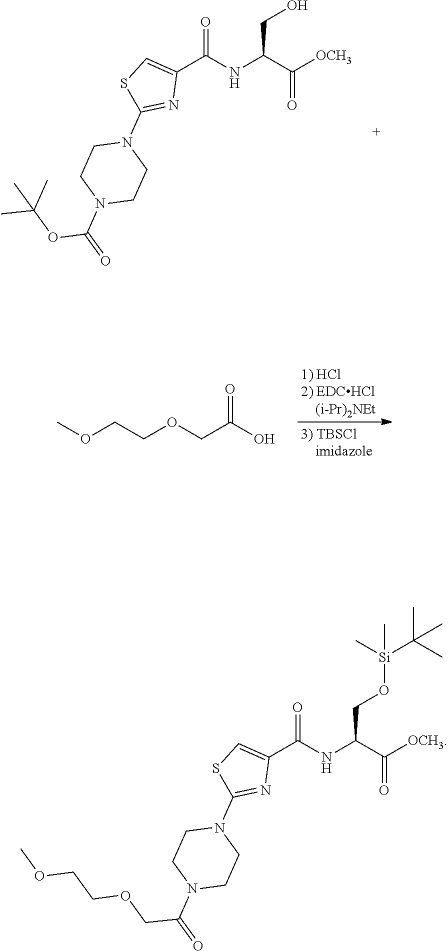

Tert-butyl (S)-4-(4-((3-hydroxy-1-methoxy-1-oxopropan-2-yl)carbamoyl)thiazol-2-yl)piperazine-1-carboxylate (1.229 g, 2.97 mmole) was dissolved in DCM (6 mL). HCl (4M solution in 1,4-dioxane, 3.00 mL, 12.0 mmole) was added and the mixture was stirred at RT for 3 hours, then was concentrated. The residue was suspended in DCM (6 mL) and 2-(2-methoxyethoxy)acetic acid (0.410 mL, 3.61 mmole) was added. N,N-Diisopropylethylamine (1.05 mL, 6.03 mmole) and EDC·HCl (0.686 g, 3.58 mmole) were added and the mixture was stirred at RT for 18 hours. Water (25 mL) was added and the mixture was extracted with DCM (3×10 mL). The combined organics were dried over magnesium sulfate, filtered and concentrated. The residue was dissolved in DCM (6 mL) and imidazole (0.225 g, 3.30 mmole) and tert-butyldimethylchlorosilane (0.500 g, 3.32 mmole) were added. The solution was stirred at RT for 2 hours and water (25 mL) was added. The mixture was extracted with DCM (3×10 mL) and the combined organics were dried over magnesium sulfate, filtered and concentrated. The crude residue was purified by silica gel chromatography to provide methyl O-(tert-butyldimethylsilyl)-N-(2-(4-(2-(2-methoxyethoxy)acetyl)piperazin-1-yl)thiazole-4-carbonyl)-L-serinate as a yellow oil (0.904 g, 56%). ¹H NMR (400 MHz, CDCl₃): δ 7.92 (d, J=8.7 Hz, 1H), 7.44 (s, 1H), 4.79 (dt, J=8.7, 3.0 Hz, 1H), 4.26 (s, 2H), 4.20-4.14 (m, 1H), 3.93-3.84 (m, 1H), 3.79-3.72 (m, 2H), 3.77 (s, 3H), 3.73-3.65 (m, 4H), 3.61-3.55 (m, 2H), 3.53 (t, J=6.3 Hz, 4H), 3.39 (s, 3H), 0.89 (s, 9H), 0.05 (s, 3H), 0.03 (s, 3H).

### Methyl O-acetyl-N—(O-(tert-butyldimethylsilyl)-N-(2-(4-(2-(2-methoxyethoxy)acetyl)piperazin-1-yl)thiazole-4-carbonyl)-L-seryl)-L-serinate (47.4)

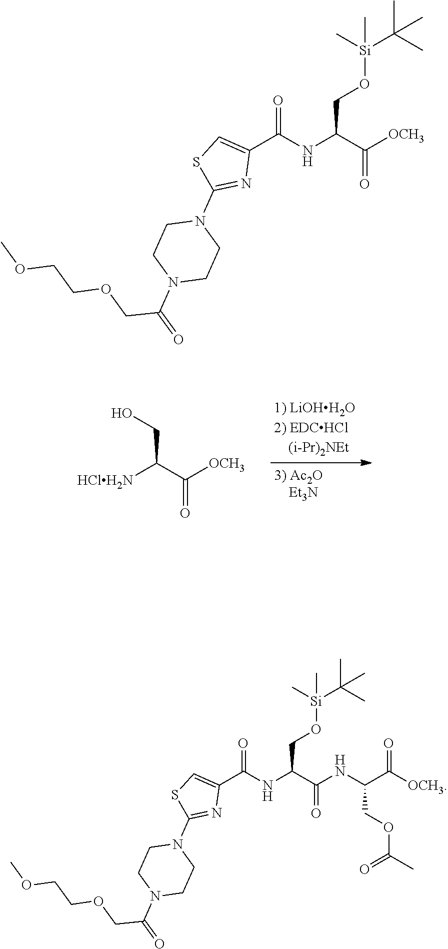

Methyl O-(tert-butyldimethylsilyl)-N-(2-(4-(2-(2-methoxyethoxy)acetyl)piperazin-1-yl)thiazole-4-carbonyl)-L-serinate (0.436 g, 0.800 mmole) was dissolved in 4/1/1 THF/methanol/water (1.6 mL). Lithium hydroxide monohydrate (0.061 g, 1.45 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in DCM (1.6 mL) and L-serine methyl ester hydrochloride (0.168 g, 1.08 mmole) was added. N,N-Diisopropylethylamine (0.290 mL, 1.66 mmole) and EDC·HCl (0.221 g, 1.15 mmole) were added and the mixture was stirred at RT for 18 hours. Water (25 mL) was added and the mixture was extracted with DCM (3×10 mL). The combined organics were dried over magnesium sulfate, filtered and concentrated. The residue was dissolved in DCM (1.6 mL) and triethylamine (0.130 mL, 0.928 mmole) and acetic anhydride (0.086 mL, 0.910 mmole) were added. The mixture was stirred at RT for 3 hours and water (25 mL) was added. The mixture was extracted with DCM (3×10 mL) and the combined organics were dried over magnesium sulfate, filtered and concentrated. The crude residue was purified by silica gel chromatography to provide methyl O-acetyl-N—(O-(tert-butyldimethylsilyl)-N-(2-(4-(2-(2-methoxyethoxy)acetyl)piperazin-1-yl)thiazole-4-carbonyl)-L-seryl)-L-serinate as a colorless oil (0.120 g, 22%). ¹H NMR (400 MHz, CDCl₃): δ 7.96 (d, J=7.0 Hz, 1H), 7.49-7.42 (m, 2H), 4.87 (dt, J=7.7, 3.8 Hz, 1H), 4.58 (td, J=7.3, 3.7 Hz, 1H), 4.48 (dd, J=11.4, 3.9 Hz, 1H), 4.32 (dd, J=11.4, 3.6 Hz, 1H), 4.25 (s, 2H), 4.22-4.15 (m, 1H), 3.77 (s, 3H), 3.76-3.72 (m, 2H), 3.72-3.65 (m, 4H), 3.61-3.46 (m, 6H), 3.39 (s, 3H), 2.02 (s, 3H), 0.93 (s, 9H), 0.15 (s, 3H), 0.14 (s, 3H).

### Methyl 2-(2-(2-(4-(2-(2-methoxyethoxy)acetyl)piperazin-1-yl)thiazole-4-carboxamido)acrylamid

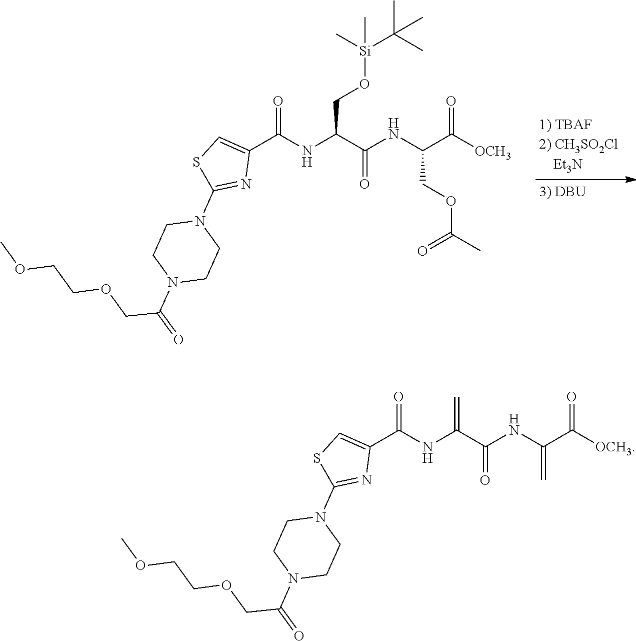 [SMILES: COCCOCC(=O)N1CCN(c2nc(C(=O)N[C@@H](CO[Si](C)(C)C(C)(C)C)C(=O)N[C@@H](COC(C)=O)C(=O)OC)cs2)CC1 ; C=C(NC(=O)c1csc(N2CCN(C(=O)COCCOC)CC2)n1)C(=O)NC(=C)C(=O)OC]

Methyl O-acetyl-N—(O-(tert-butyldimethylsilyl)-N-(2-(4-(2-(2-methoxyethoxy)acetyl)piperazin-1-yl)thiazole-4-carbonyl)-L-seryl)-L-serinate (0.120 g, 0.178 mmole) was dissolved in THE (1.8 mL). TBAF (1M solution in THF, 0.450 mL, 0.450 mmole) was added and the solution was stirred at RT for 2 hours. Water (25 mL) and sat. aq. sodium chloride (10 mL) were added and the mixture was extracted with ethyl acetate (3×10 mL). The combined organics were dried over magnesium sulfate, filtered and concentrated. The residue was dissolved in DCM (1.8 mL) and cooled to 0° C. Triethylamine (0.040 mL, 0.285 mmole) and methanesulfonyl chloride (0.026 mL, 0.336 mmole) were added and the mixture was stirred at 0° for 60 minutes and water (25 mL) was added. The mixture was extracted with DCM (3×10 mL) and the combined organics were dried over magnesium sulfate, filtered and concentrated. The residue was dissolved in THE (2 mL) and cooled to 0° C. DBU (0.040 mL, 0.267 mmole) was added and the mixture was stirred at 0° for 45 minutes. Water (25 mL) was added and the mixture was extracted with DCM (3×10 mL). The combined organics were dried over magnesium sulfate, filtered and concentrated. The crude residue was purified by silica gel chromatography to provide methyl 2-(2-(2-(4-(2-(2-methoxyethoxy)acetyl)piperazin-1-yl)thiazole-4-carboxamido)acrylamido)acrylate as a pale yellow solid (0.026 g, 30%). ¹H NMR (400 MHz, CDCl₃): δ 9.74 (s, 1H), 8.52 (s, 1H), 7.48 (s, 1H), 6.73 (d, J=2.1 Hz, 1H), 6.66 (s, 1H), 6.01 (d, J=1.3 Hz, 1H), 5.44 (t, J=1.9 Hz, 1H), 4.26 (s, 2H), 3.90 (s, 3H), 3.77 (s, 2H), 3.74-3.67 (m, 4H), 3.63-3.56 (m, 4H), 3.49 (d, J=12.0 Hz, 2H), 3.39 (s, 3H).

### Compound 48: Methyl 2-(2-(2-(4-(6-chlorohexanamido)phenyl)thiazole-4-carboxamido)acrylamido)acrylate

### Tert-butyl (4-carbamothioylphenyl)carbamate (48.1)

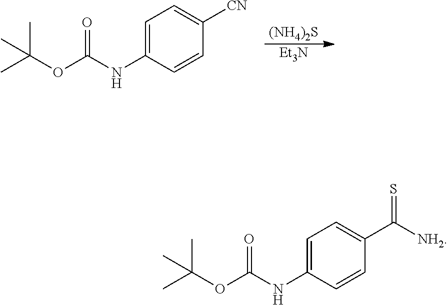 [SMILES: "Et3N" ; CC(C)(C)OC(=O)Nc1ccc(C#N)cc1 ; "(NH4)2S" ; CC(C)(C)OC(=O)Nc1ccc(C(N)=S)cc1]

Tert-butyl (4-cyanophenyl)carbamate (5.024 g, 23.0 mmole) was dissolved in pyridine (23 mL). Triethylamine (3.60 mL, 25.7 mmole) and ammonium sulfide (40% aqueous solution, 4.70 mL, 27.5 mmole) were added and the mixture was heated to 50° C. for 24 hours, then was cooled to RT and concentrated. The residue was dissolved in ethyl acetate (50 mL) and the solution was washed sequentially with 1N aq. HCl (2×25 mL), water (1×25 mL) and sat. aq. sodium chloride (1×25 mL). The organics were dried over magnesium sulfate, filtered and concentrated. The residue was suspended in diethyl ether (15 mL) and the solid was collected by filtration, then allowed to air dry to provide tert-butyl (4-carbamothioylphenyl)carbamate as a yellow solid (4.826 g, 83%). ¹H NMR (400 MHz, d₆-DMSO): δ 9.64 (d, J=4.5 Hz, 2H), 9.30 (s, 1H), 7.93-7.82 (m, 2H), 7.51-7.40 (m, 2H), 1.48 (s, 9H).

### Ethyl 2-(4-aminophenyl)thiazole-4-carboxylate (48.2)

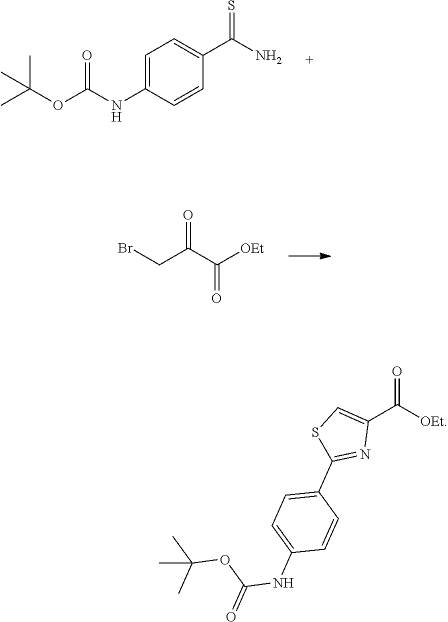 [SMILES: CC(C)(C)OC(=O)Nc1ccc(C(N)=S)cc1 ; CCOC(=O)C(=O)CBr ; CCOC(=O)c1csc(-c2ccc(NC(=O)OC(C)(C)C)cc2)n1]

Tert-butyl (4-carbamothioylphenyl)carbamate (4.826 g, 19.1 mmole) was dissolved in ethanol (38 mL). Ethyl bromopyruvate (2.90 mL, 23.1 mmole) was added and the mixture was heated to 80° C. for 4 hours. The mixture was cooled to RT and concentrated. The residue was suspended in DCM (38 mL) and triethylamine (5.40 mL, 38.5 mmole) and di-tert-butyl dicarbonate (4.608 g, 21.1 mmole) were added. The mixture was stirred at RT for 18 hours and water (75 mL) was added. The two layers were separated and the aqueous layer was extracted with DCM (2×15 mL). The combined organics were dried over magnesium sulfate, filtered and concentrated. The crude residue was purified by silica gel chromatography to provide ethyl 2-(4-((tert-butoxycarbonyl)amino)phenyl)thiazole-4-carboxylate as a yellow solid (0.983 g, 15%) and ethyl 2-(4-aminophenyl)thiazole-4-carboxylate (Compound 48.2) as an orange solid (2.646 g, 56%). ¹H NMR (400 MHz, CDCl₃): δ 8.03 (s, 1H), 7.85-7.78 (m, 2H), 6.74-6.65 (m, 2H), 4.43 (q, J=7.1 Hz, 2H), 3.97 (s, 2H), 1.42 (t, J=7.1 Hz, 3H).

### Ethyl 2-(4-(6-chlorohexanamido)phenyl)thiazole-4-carboxylate (48.3)

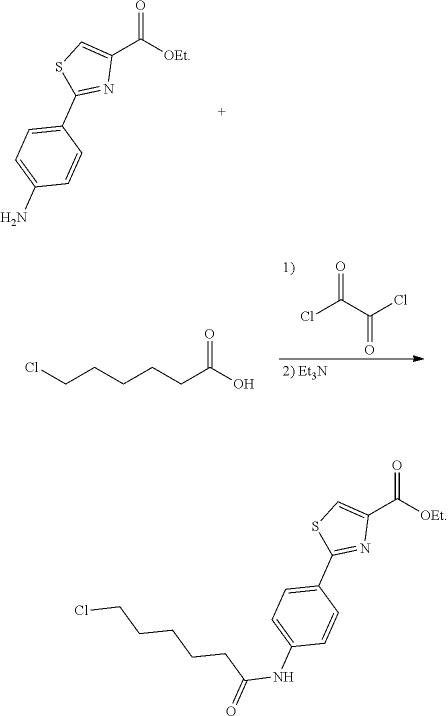 [SMILES: CCOC(=O)c1csc(-c2ccc(N)cc2)n1 ; O=C(O)CCCCCCl ; O=C(Cl)C(=O)Cl ; CCOC(=O)c1csc(-c2ccc(NC(=O)CCCCCCl)cc2)n1]

6-Chlorohexanoic acid 1.573 g, 10.4 mmole) was dissolved in DCM (16 mL) and DMF (1 drop) was added. Oxalyl chloride (0.910 mL, 10.4 mmole) was added dropwise (gas evolution was observed) and the solution was stirred at RT for 3 hours, then was concentrated. Ethyl 2-(4-aminophenyl)thiazole-4-carboxylate (1.986 g, 8.00 mmole) was dissolved in DCM (8 mL) and cooled to 0° C. Triethylamine (1.70 mL, 12.1 mmole) was added followed by a solution of the acid chloride generated above in DCM (8 mL). The resulting mixture was allowed to warm to RT and stir for 18 hours. Water (50 mL) was added and the two layers were separated. The aqueous layer was extracted with DCM (2×15 mL) and the combined organics were dried over magnesium sulfate, filtered and concentrated. The crude residue was purified by silica gel chromatography to provide ethyl 2-(4-(6-chlorohexanamido)phenyl)thiazole-4-carboxylate as a yellow solid (2.441 g, 80%). ¹H NMR (400 MHz, CDCl₃): δ 8.12 (s, 1H), 8.01-7.92 (m, 2H), 7.68 (d, J=8.4 Hz, 2H), 7.59 (s, 1H), 4.45 (q, J=7.1 Hz, 2H), 3.55 (t, J=6.6 Hz, 2H), 2.42 (t, J=7.4 Hz, 2H), 1.79 (tdd, J=15.4, 10.9, 6.9 Hz, 4H), 1.60-1.47 (m, 2H), 1.43 (t, J=7.1 Hz, 3H).

### Methyl O-(tert-butyldimethylsilyl)-N-(2-(4-(6-chlorohexanamido)phenyl)thiazole-4-carbonyl)-L-serinate (48.4)

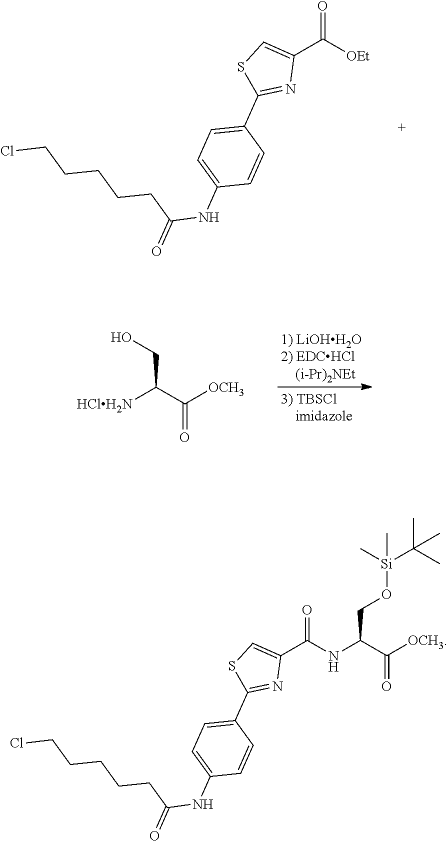

Ethyl 2-(4-(6-chlorohexanamido)phenyl)thiazole-4-carboxylate (2.441 g 6.41 mmole) was dissolved in 4/1/1 THF/methanol/water (13 mL). Lithium hydroxide monohydrate (0.414 g, 9.87 mmole) was added and the mixture was stirred at RT for 2 hours. Water (50 mL) was added and the solution was treated with 1N aq. HCl to pH=4. The mixture was extracted with ethyl acetate (3×15 mL). The combined organics were filtered to remove the precipitated acid, and the filtrate was dried over magnesium sulfate, filtered and concentrated. The residue was combined with the filtered solid and suspended in DCM (13 mL). L-Serine methyl ester hydrochloride (1.306 g, 8.39 mmole) was added followed by N,N-diisopropylethylamine (2.30 mL, 13.2 mmole) and EDC·HCl (1.597 g, 8.33 mmole). The mixture was stirred at RT for 2 days and water (50 mL) was added. The two layers were separated and the aqueous layer was extracted with DCM (2×15 mL). The combined organics were dried over magnesium sulfate, filtered and concentrated. The residue was dissolved in DCM (13 mL) and imidazole (0.479 g, 7.04 mmole) and tert-butyldimethylchlorosilane (1.059 g, 7.03 mmole) were added. The mixture was stirred at RT for 3 hours and water (50 mL) was added. The two layers were separated and the aqueous layer was extracted with DCM (2×10 mL). The combined organics were dried over magnesium sulfate, filtered and concentrated. The crude residue was purified by silica gel chromatography to provide methyl O-(tert-butyldimethylsilyl)-N-(2-(4-(6-chlorohexanamido)phenyl)thiazole-4-carbonyl)-L-serinate as a yellow oil (1.991 g, 55%). ¹H NMR (400 MHz, CDCl₃): δ 8.21 (d, J=8.7 Hz, 1H), 8.04 (s, 1H), 7.93-7.84 (m, 2H), 7.67-7.56 (m, 3H), 4.85 (dt, J=8.7, 3.0 Hz, 1H), 4.22 (dd, J=10.0, 2.7 Hz, 1H), 3.95 (dd, J=10.1, 3.4 Hz, 1H), 3.79 (s, 3H), 3.56 (t, J=6.6 Hz, 2H), 2.41 (t, J=7.4 Hz, 2H), 1.89-1.72 (m, 4H), 1.60-1.50 (m, 2H), 0.92 (s, 9H), 0.08 (s, 3H), 0.06 (s, 3H).

### Methyl O-acetyl-N—(O-(tert-butyldimethylsilyl)-N-(2-(4-(6-chlorohexanamido)phenyl)thiazole-4-carbonyl)-L-seryl)-L-serinate (48.5)

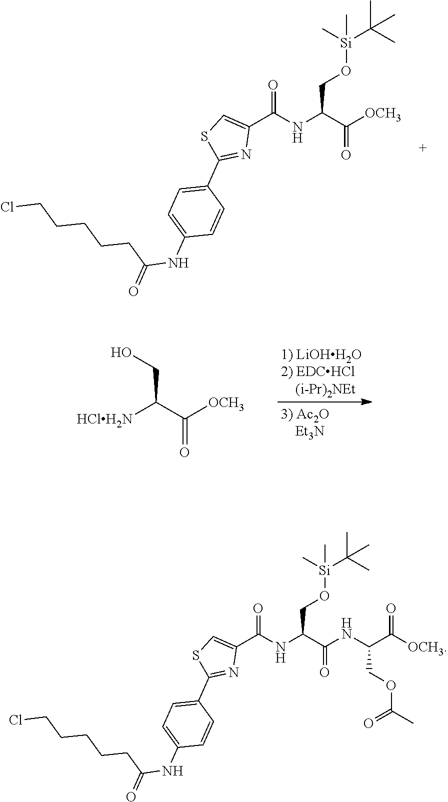

Methyl O-(tert-butyldimethylsilyl)-N-(2-(4-(6-chlorohexanamido)phenyl)thiazole-4-carbonyl)-L-serinate (1.991 g, 3.50 mmole) was dissolved in 4/1/1 THF/methanol/water (7 mL). Lithium hydroxide monohydrate (0.223 g, 5.31 mmole) was added and the mixture was stirred at RT for 4 hours. Water (50 mL) was added and the solution was treated with 1N aq. HCl to pH=4. The mixture was extracted with ethyl acetate (3×15 mL) and the combined organics were dried over magnesium sulfate, filtered and concentrated. The residue was dissolved in DCM (7 mL) and L-serine methyl ester hydrochloride (0.707 g, 4.54 mmole) was added. N,N-Diisopropylethylamine (1.25 mL, 7.18 mmole) and EDC·HCl (0.878 g, 4.58 mmole) were added and the mixture was stirred at RT for 18 hours. Water (25 mL) was added and the mixture was extracted with DCM (3×10 mL). The combined organics were dried over magnesium sulfate, filtered and concentrated. The residue was dissolved in DCM (7 mL) and triethylamine (0.540 mL, 3.85 mmole) and acetic anhydride (0.370 mL, 3.91 mmole) were added. The mixture was stirred at RT for 3 hours and water (25 mL) was added. The mixture was extracted with DCM (3×10 mL) and the combined organics were dried over magnesium sulfate, filtered and concentrated. The crude residue was purified by silica gel chromatography to provide methyl 0-acetyl-N-(0-(tert-butyldimethylsilyl)-N-(2-(4-(6-chlorohexanamido)phenyl)thiazole-4-carbonyl)-L-seryl)-L-serinate as a pale yellow oil (1.004 g, 41%). ¹H NMR (400 MHz, CDCl₃): δ 8.25 (d, J=7.0 Hz, 1H), 8.07 (d, J=3.7 Hz, 1H), 7.94-7.87 (m, 2H), 7.63 (d, J=8.5 Hz, 2H), 7.53 (d, J=10.2 Hz, 1H), 7.48 (d, J=7.7 Hz, 1H), 4.89 (tt, J=6.2, 3.7 Hz, 1H), 4.67 (td, J=7.2, 3.7 Hz, 1H), 4.53-4.46 (m, 1H), 4.34 (dd, J=11.4, 3.6 Hz, 1H), 4.22 (dt, J=9.9, 3.8 Hz, 1H), 3.84-3.79 (m, 1H), 3.77 (d, J=3.3 Hz, 3H), 3.56 (t, J=6.6 Hz, 2H), 2.42 (t, J=7.4 Hz, 2H), 2.01 (s, 3H), 1.88-1.72 (m, 4H), 1.61-1.49 (m, 2H), 0.96 (s, 9H), 0.16 (s, 3H), 0.15 (s, 3H).

### Methyl 2-(2-(2-(4-(6-chlorohexanamido)phenyl)thiazole-4-carboxamido)acrylamido)acrylate (48)

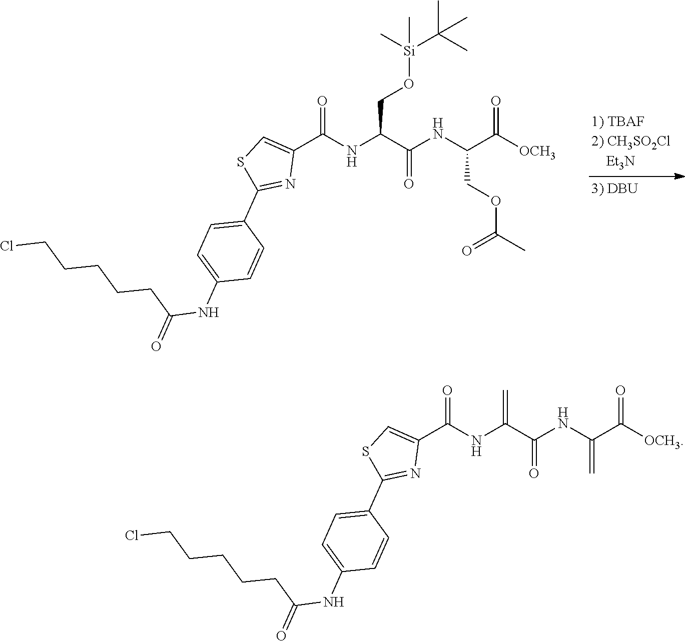 [SMILES: COC(=O)[C@H](COC(C)=O)NC(=O)[C@H](CO[Si](C)(C)C(C)(C)C)NC(=O)c1csc(-c2ccc(NC(=O)CCCCCCl)cc2)n1 ; C=C(NC(=O)c1csc(-c2ccc(NC(=O)CCCCCCl)cc2)n1)C(=O)NC(=C)C(=O)OC]

Methyl 0-acetyl-N-(0-(tert-butyldimethylsilyl)-N-(2-(4-(6-chlorohexanamido)phenyl)thiazole-4-carbonyl)-L-seryl)-L-serinate (TLS-025-5-1) was dissolved in THF. TBAF (1M solution in THF) was added and the solution was stirred at RT for 2 hours. Water (25 mL) was added and the mixture was extracted with ethyl acetate (3×10 mL). The combined organics were dried over magnesium sulfate, filtered and concentrated. The residue was dissolved in DCM and cooled to 0° C. Triethylamine and methanesulfonyl chloride were added and the mixture was stirred at 0° C. for 90 minutes. Water (25 mL) was added and the mixture was extracted with DCM (3×10 mL). The combined organics were dried over magnesium sulfate, filtered and concentrated. The residue was dissolved in THE (2.9 mL) and cooled to 0° C. DBU was added and the mixture was stirred at 0° C. for 60 minutes. Water (25 mL) was added and the mixture was extracted with ethyl acetate (3×10 mL). The combined organics were dried over magnesium sulfate, filtered and concentrated. The crude residue was purified by silica gel chromatography (Isco CombiPrep, 12 g RediSep column, 20-50% ethyl acetate/hexane gradient) to provide methyl 2-(2-(2-(4-(6-chlorohexanamido)phenyl)thiazole-4-carboxamido)acrylamido)acrylate (TLS-025-11-1) as a white solid (0.356 g, 49%).

### Compound 49: Methyl 2-(2-(2-(4-(13,13-dimethyl-3,11-dioxo-4,7,12-trioxa-2,10-diazatetradecyl)phenyl)thiazole-4-carboxamido)acrylamido)acrylate

### Ethyl 2-(4-(13,13-dimethyl-3,11-dioxo-4,7,12-trioxa-2,10-diazatetradecyl)phenyl)thiazole-4-carboxylate (49.1)

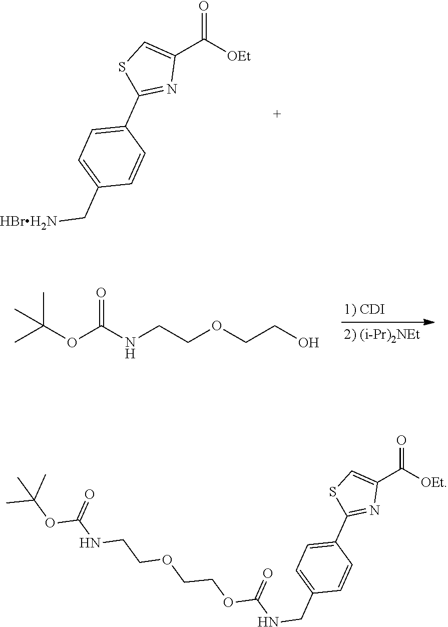

Tert-butyl (2-(2-hydroxyethoxy)ethyl)carbamate (0.496 g, 2.42 mmole) was dissolved in THE (4.4 mL). CDI (0.418 g, 2.58 mmole) was added slowly and the solution was stirred at RT for 4 hours, then was diluted with ethyl acetate (10 mL). Water (25 mL) was added and the two layers were separated. The aqueous layer was extracted with ethyl acetate (2×10 mL) and the combined organics were dried over magnesium sulfate, filtered and concentrated. The residue was dissolved in DMF (4.4 mL) and ethyl 2-(4-(aminomethyl)phenyl)thiazole-4-carboxylate hydrobromide (Example 45.1, 0.752 g, 2.19 mmole) was added. N,N-Diisopropylethylamine (0.460 mL, 2.64 mmole) was added and the mixture was warmed to 50° C. for 18 hours, then was cooled to RT and diluted with ethyl acetate (25 mL). The mixture was washed with water (3×15 mL) and the organics were dried over magnesium sulfate, filtered and concentrated. The crude residue was purified by silica gel chromatography to provide ethyl 2-(4-(13,13-dimethyl-3,11-dioxo-4,7,12-trioxa-2,10-diazatetradecyl)phenyl)thiazole-4-carboxylate as an orange solid (0.873 g, 81%). ¹H NMR (400 MHz, CDCl₃): δ 8.15 (s, 1H), 7.97 (d, J=8.3 Hz, 2H), 7.38 (d, J=8.0 Hz, 2H), 5.33 (s, 1H), 4.98 (s, 1H), 4.50-4.38 (m, 4H), 4.31-4.23 (m, 2H), 3.71-3.62 (m, 2H), 3.62-3.50 (m, 2H), 3.32 (d, J=5.8 Hz, 2H), 1.49-1.38 (m, 12H).

### Methyl O-(tert-butyldimethylsilyl)-N-(2-(4-(13,13-dimethyl-3,11-dioxo-4,7,12-trioxa-2,10-diazatetradecyl)phenyl)thiazole-4-carbonyl)-L-serinate (49.2)

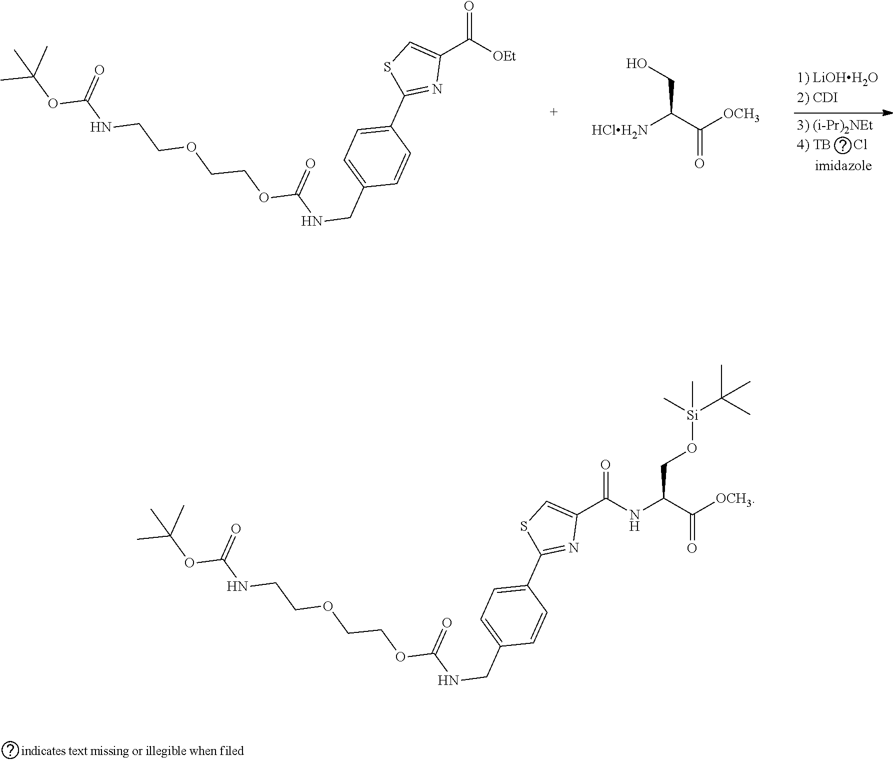

Ethyl 2-(4-(13,13-dimethyl-3,11-dioxo-4,7,12-trioxa-2,10-diazatetradecyl)phenyl)thiazole-4-carboxylate (0.873 g, 1.77 mmole) was dissolved in 4/1/1 THF/methanol/water (3.5 mL). Lithium hydroxide monohydrate (0.111 g, 2.65 mmole) was added and the mixture was stirred at RT for 4 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was suspended in DCM (3.5 mL) and CDI (0.317 g, 1.95 mmole) was added portionwise (mixture became homogeneous and gas evolution was observed). The solution was stirred for 60 minutes and L-serine methyl ester hydrochloride (0.365 g, 2.35 mmole) and N,N-diisopropylethylamine (0.620 mL, 3.56 mmole) were added. The mixture was stirred at RT for 2 ½ days and imidazole (0.136 g, 2.00 mmole) and tert-butyldimethylchlorosilane (0.307 g, 2.04 mmole) were added. The mixture was stirred at RT for 4 hours and water (25 mL) was added. The mixture was extracted with DCM (3×10 mL) and the combined organics were dried over magnesium sulfate, filtered and concentrated. The crude residue was purified by silica gel chromatography to provide methyl O-(tert-butyldimethylsilyl)-N-(2-(4-(13,13-dimethyl-3,11-dioxo-4,7,12-trioxa-2,10-diazatetradecyl)phenyl)thiazole-4-carbonyl)-L-serinate as a colorless oil (0.614 g, 51%). ¹H NMR (400 MHz, CDCl₃): δ 8.21 (d, J=8.7 Hz, 1H), 8.10 (s, 1H), 7.97-7.88 (m, 2H), 7.42-7.33 (m, 2H), 5.31 (s, 1H), 4.97 (s, 1H), 4.86 (dt, J=8.7, 3.1 Hz, 1H), 4.44 (d, J=6.1 Hz, 2H), 4.32-4.24 (m, 2H), 4.21 (dd, J=10.1, 2.7 Hz, 1H), 4.00-3.91 (m, 1H), 3.79 (s, 3H), 3.71-3.64 (m, 2H), 3.55 (q, J=7.6, 6.5 Hz, 2H), 3.38-3.25 (m, 2H), 1.43 (s, 9H), 0.92 (s, 8H), 0.08 (s, 3H), 0.06 (s, 3H).

### Methyl O-acetyl-N—(O-(tert-butyldimethylsilyl)-N-(2-(4-(13,13-dimethyl-3,11-dioxo-4,7,12-trioxa-2,10-diazatetradecyl)phenyl)thiazole-4-carbonyl)-L-seryl)-L-serinate (49.3)

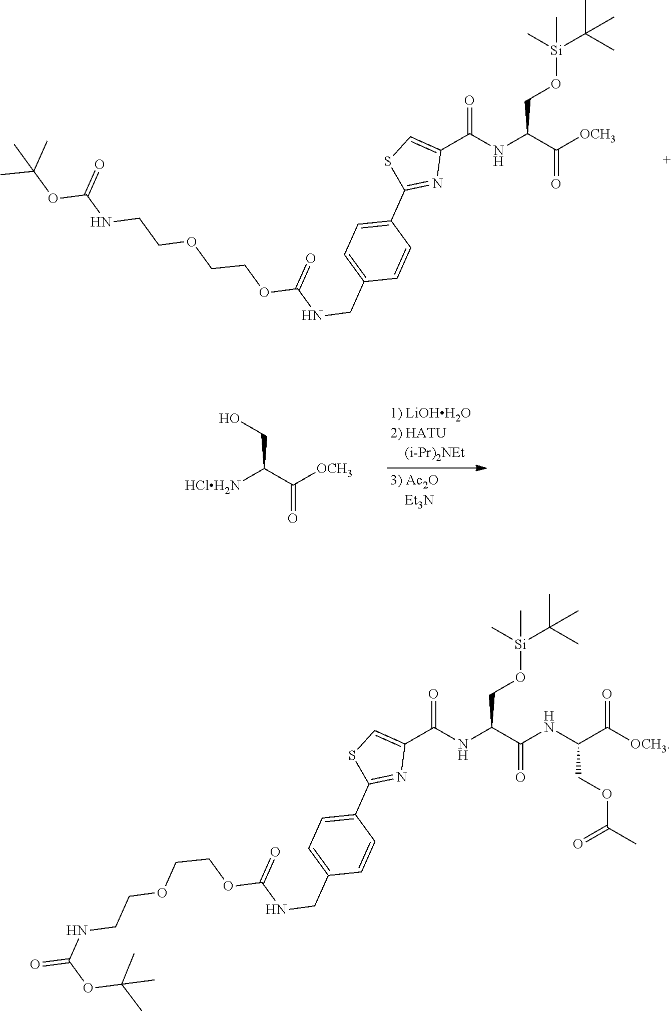

Methyl O-(tert-butyldimethylsilyl)-N-(2-(4-(13,13-dimethyl-3,11-dioxo-4,7,12-trioxa-2,10-diazatetradecyl)phenyl)thiazole-4-carbonyl)-L-serinate (0.614 g, 0.902 mmole) was dissolved in 4/1/1 THF/methanol/water (2 mL). Lithium hydroxide monohydrate (0.066 g, 1.57 mmole) was added and the mixture was stirred at RT for 90 minutes. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in DCM (2 mL) and L-serine methyl ester hydrochloride (0.187 g, 1.20 mmole) was added. N,N-Diisopropylethylamine (0.320 mL, 1.84 mmole) and HATU (0.410 g, 1.08 mmole) were added and the mixture was stirred at RT for 18 hours. Water (25 mL) was added and the mixture was extracted with DCM (3×10 mL). The combined organics were dried over magnesium sulfate, filtered and concentrated. The residue was dissolved in DCM (2 mL). Triethylamine (0.140 mL, 0.999 mmole) and acetic anhydride (0.094 mL, 0.994 mmole) were added and the mixture was stirred at RT for 4 hours. Water (25 mL) was added and the mixture was extracted with DCM (3×10 mL). The combined organics were dried over magnesium sulfate, filtered and concentrated. The crude residue was purified by silica gel chromatography to provide methyl O-acetyl-N—(O-(tert-butyldimethylsilyl)-N-(2-(4-(13,13-dimethyl-3,11-dioxo-4,7,12-trioxa-2,10-diazatetradecyl)phenyl)thiazole-4-carbonyl)-L-seryl)-L-serinate as a pale yellow oil (0.530 g, 73%). ¹H NMR (400 MHz, CDCl₃): δ 8.26 (dd, J=7.2, 2.5 Hz, 1H), 8.24-8.19 (m, 1H), 8.11 (d, J=2.5 Hz, 1H), 7.94 (dd, J=8.4, 1.8 Hz, 2H), 7.48 (d, J=7.8 Hz, 1H), 7.38 (d, J=8.0 Hz, 2H), 6.54-6.45 (m, 1H), 4.97 (s, 1H), 4.90 (dq, J=7.0, 3.5 Hz, 1H), 4.67 (td, J=7.3, 3.7 Hz, 1H), 4.50 (dd, J=11.4, 3.9 Hz, 1H), 4.43 (d, J=6.3 Hz, 2H), 4.34 (dd, J=11.4, 3.6 Hz, 1H), 4.30-4.25 (m, 2H), 4.25-4.18 (m, 1H), 3.85-3.79 (m, 1H), 3.77 (d, J=4.3 Hz, 3H), 3.68 (dd, J=5.7, 3.6 Hz, 2H), 3.56 (t, J=5.3 Hz, 2H), 3.33 (d, J=6.0 Hz, 2H), 2.02 (s, 3H), 1.43 (s, 9H), 0.96 (s, 9H), 0.17 (s, 3H), 0.15 (s, 3H).

### Methyl 2-(2-(2-(4-(13,13-dimethyl-3,11-dioxo-4,7,12-trioxa-2,10-diazatetradecyl)phenyl)thiazole-4-carboxamido)acrylamido)acrylate (49)

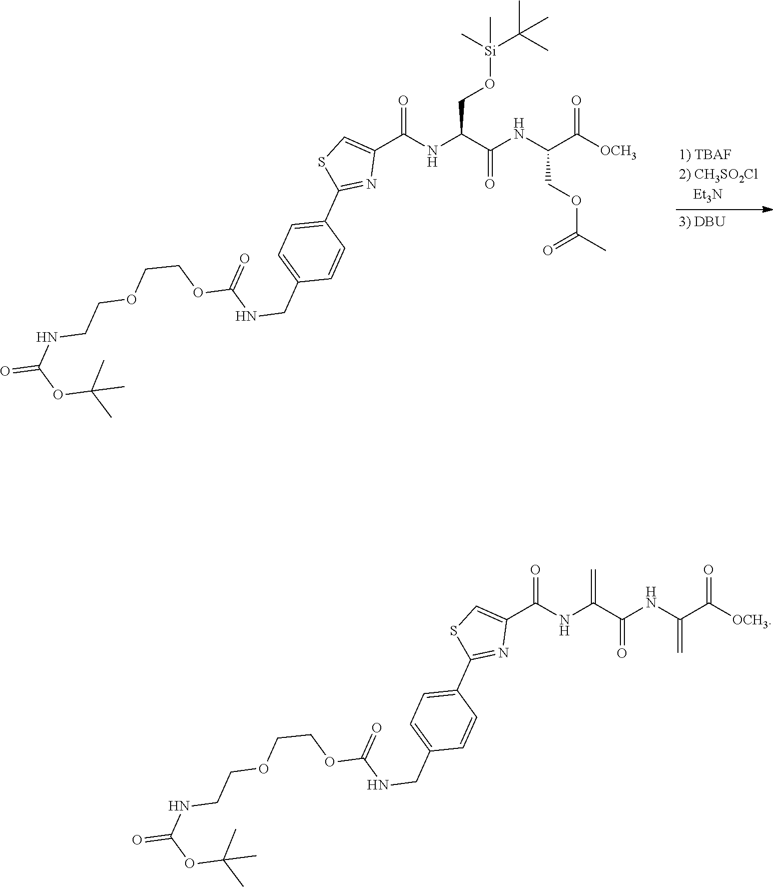 [SMILES: COC(=O)[C@H](COC(C)=O)NC(=O)[C@H](CO[Si](C)(C)C(C)(C)C)NC(=O)c1csc(-c2ccc(CNC(=O)OCCOCCNC(=O)OC(C)(C)C)cc2)n1 ; C=C(NC(=O)c1csc(-c2ccc(CNC(=O)OCCOCCNC(=O)OC(C)(C)C)cc2)n1)C(=O)NC(=C)C(=O)OC]

Methyl O-acetyl-N-(0-(tert-butyldimethylsilyl)-N-(2-(4-(13,13-dimethyl-3,11-dioxo-4,7,12-trioxa-2,10-diazatetradecyl)phenyl)thiazole-4-carbonyl)-L-seryl)-L-serinate (0.530 g, 0.654 mmoles) was dissolved in THF (1.3 mL). TBAF (1M solution in THF, 1.65 mL, 1.65 mmole) was added and the solution was stirred at RT for 2 hours. Water (25 mL) was added and the mixture was extracted with ethyl acetate (3×10 mL). The combined organics were dried over magnesium sulfate, filtered and concentrated. The residue was dissolved in DCM (2.6 mL) and cooled to 0° C. Triethylamine (0.140 mL, 0.999 mmole) and methanesulfonyl chloride (0.076 mL, 0.982 mmole) were added and the mixture was stirred at 0° for 90 minutes. Water (25 mL) was added and the mixture was extracted with DCM (3×10 mL). The combined organics were dried over magnesium sulfate, filtered and concentrated. The residue was dissolved in THE (1.3 mL) and cooled to 0° C. DBU (0.150 mL, 1.00 mmole) was added and the mixture was stirred at 0° for 60 minutes. Water (25 mL) was added and the mixture was extracted with ethyl acetate (3×10 mL). The combined organics were dried over magnesium sulfate, filtered and concentrated. The crude residue was purified by silica gel chromatography to provide methyl 2-(2-(2-(4-(13,13-dimethyl-3,11-dioxo-4,7,12-trioxa-2,10-diazatetradecyl)phenyl)thiazole-4-carboxamido)acrylamido)acrylate as a white solid (0.121 g, 30%). ¹H NMR (400 MHz, CDCl₃): δ 10.01 (s, 1H), 8.55 (s, 1H), 8.14 (s, 1H), 8.03-7.93 (m, 2H), 7.40 (d, J=8.2 Hz, 2H), 6.78 (d, J=2.2 Hz, 1H), 6.71 (s, 1H), 6.03 (d, J=1.3 Hz, 1H), 5.50 (t, J=1.9 Hz, 1H), 5.27 (s, 1H), 4.96 (s, 1H), 4.44 (d, J=6.1 Hz, 2H), 4.33-4.23 (m, 2H), 3.91 (s, 3H), 3.73-3.62 (m, 2H), 3.56 (t, J=5.2 Hz, 2H), 3.39-3.23 (m, 2H), 1.43 (s, 9H).

### Compound 50: Methyl 2-(2-(2-(4-(((((6-azidohexyl)oxy)carbonyl)amino)methyl)phenyl)thiazole-4-carboxamido)acrylamido)acrylate

### Ethyl 2-(4-(((((6-azidohexyl)oxy)carbonyl)amino)methyl)phenyl)thiazole-4-carboxylate (50.1)

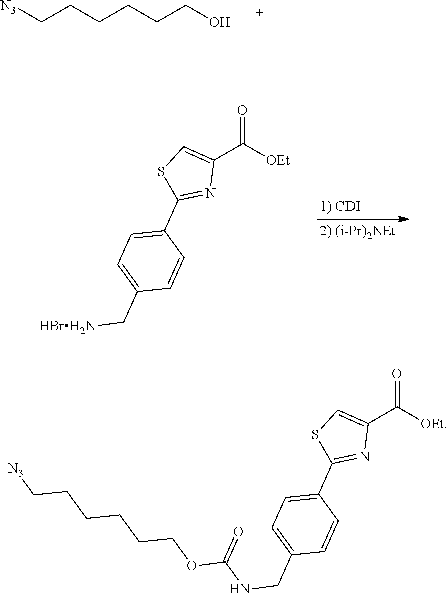

6-Azidohexan-1-ol (0.794 g, 5.55 mmole) was dissolved in THE (11 mL). CDI (0.904 g, 5.58 mmole) was added slowly and the solution was stirred at RT for 18 hours. Water (25 mL) was added and the mixture was extracted with ethyl acetate (3×10 mL). The combined organics were dried over magnesium sulfate, filtered and concentrated. The residue was dissolved in DMF (6 mL) and ethyl 2-(4-(aminomethyl)phenyl)thiazole-4-carboxylate hydrobromide (Example 45.1, 1.992 g, 5.80 mmole) was added. N,N-Diisopropylethylamine (1.20 mL, 6.89 mmole) was added and the mixture was warmed to 50° C. and stirred for 5 hours, then was cooled to RT and diluted with ethyl acetate (50 mL). The mixture was washed with water (3×20 mL) and the organics were dried over magnesium sulfate, filtered and concentrated. The crude residue was purified by silica gel chromatography to provide ethyl 2-(4-(((((6-azidohexyl)oxy)carbonyl)amino)methyl)phenyl)thiazole-4-carboxylate as a yellow solid (1.125 g, 47%). ¹H NMR (400 MHz, CDCl₃): δ 8.15 (s, 1H), 8.02-7.94 (m, 2H), 7.37 (d, J=8.2 Hz, 2H), 5.10 (s, 1H), 4.50-4.35 (m, 4H), 4.15-4.06 (m, 2H), 3.28 (dt, J=10.9, 6.9 Hz, 2H), 1.72-1.52 (m, 4H), 1.42 (q, J=6.3, 5.5 Hz, 7H).

### Methyl N-(2-(4-(((((6-azidohexyl)oxy)carbonyl)amino)methyl)phenyl)thiazole-4-carbonyl)-O-(tert-butyldimethylsilyl)-L-serinate (50.2)

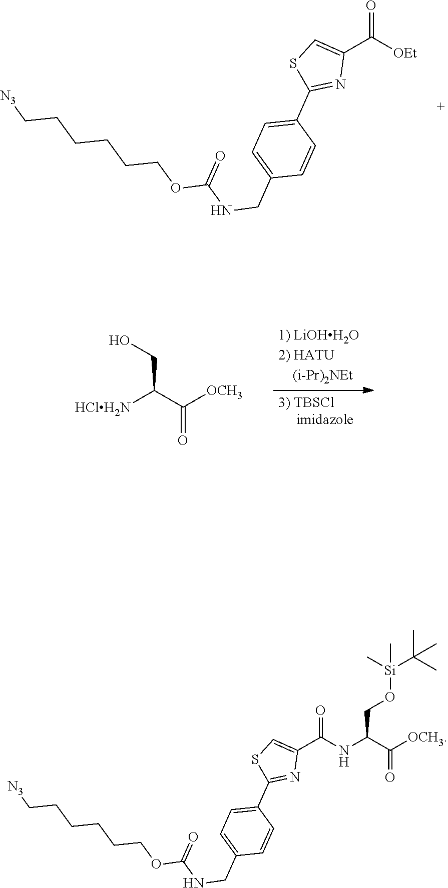

Ethyl 2-(4-(((((6-azidohexyl)oxy)carbonyl)amino)methyl)phenyl)thiazole-4-carboxylate (1.125 g, 2.61 mmole) was dissolved in 4/1/1 THF/methanol/water (5.2 mL). Lithium hydroxide monohydrate (0.171, 4.08 mmole) was added and the mixture was stirred at RT for 3 hours. Water (25 mL) was added and the mixture was treated with 1N aq. HCl to pH=4. The mixture was extracted with ethyl acetate (3×15 mL) and the combined organics were dried over magnesium sulfate, filtered and concentrated. The residue was dissolved in DCM (5.2 mL) and L-serine methyl ester hydrochloride (0.525 g, 3.37 mmole) was added. N,N-Diisopropylethylamine (0.910 mL, 5.22 mmole) and HATU (1.186 g, 3.12 mmole) were added and the mixture was stirred at RT for 18 hours. Water (25 mL) was added and the mixture was extracted with DCM (3×10 mL). The combined organics were dried over magnesium sulfate, filtered and concentrated. The residue was dissolved in DCM (5.2 mL) and imidazole (0.194 g, 2.85 mmole) and tert-butyldimethylchlorosilane (0.444 g, 2.95 mmole) were added. The mixture was stirred at RT for 3 hours and water (25 mL) was added. The mixture was extracted with DCM (3×10 mL) and the combined organics were dried over magnesium sulfate, filtered and concentrated. The crude residue was purified by silica gel chromatography to provide methyl N-(2-(4-(((((6-azidohexyl)oxy)carbonyl)amino)methyl)phenyl)thiazole-4-carbonyl)-O-(tert-butyldimethylsilyl)-L-serinate as a pale yellow oil (1.330 g, 82%). ¹H NMR (400 MHz, CDCl₃): δ 8.21 (d, J=8.7 Hz, 1H), 8.09 (s, 1H), 7.98-7.89 (m, 2H), 7.38 (d, J=7.9 Hz, 2H), 5.08 (s, 1H), 4.86 (dt, J=8.7, 3.0 Hz, 1H), 4.43 (d, J=6.1 Hz, 2H), 4.21 (dd, J=10.0, 2.7 Hz, 1H), 4.12 (td, J=6.9, 2.7 Hz, 2H), 3.95 (dd, J=10.1, 3.4 Hz, 1H), 3.79 (s, 3H), 3.27 (t, J=5.8 Hz, 2H), 1.66 (d, J=6.9 Hz, 4H), 1.41 (s, 4H), 0.92 (s, 9H), 0.08 (s, 3H), 0.06 (s, 3H).

### Methyl O-acetyl-N—(N-(2-(4-(((((6-azidohexyl)oxy)carbonyl)amino)methyl)phenyl)thiazole-4-carbonyl)-O-(tert-butyldimethylsilyl)-L-seryl)-L-serinate (50.3)

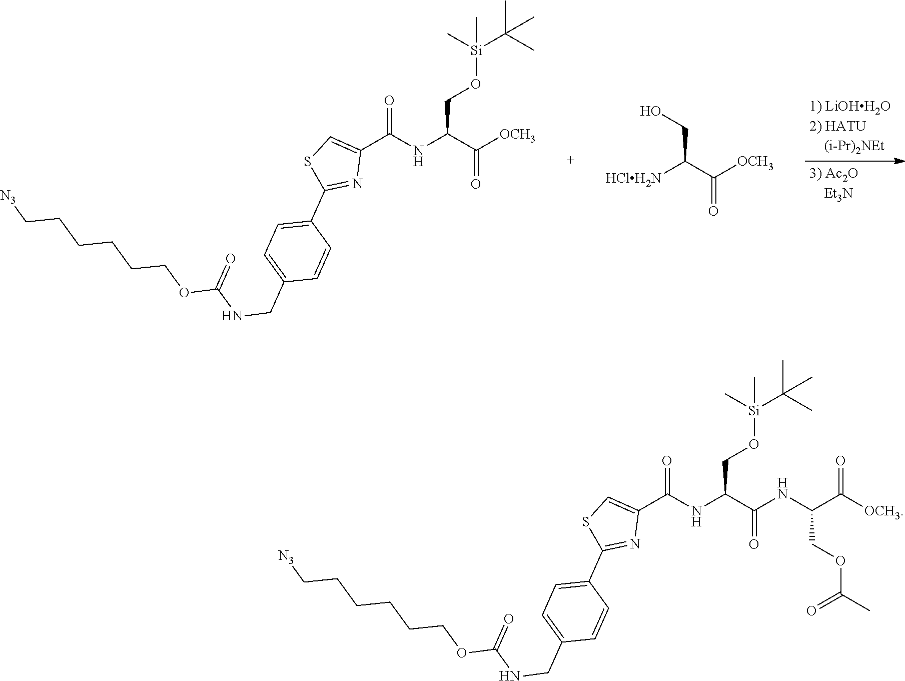

Methyl N-(2-(4-(((((6-azidohexyl)oxy)carbonyl)amino)methyl)phenyl)thiazole-4-carbonyl)-O-(tert-butyldimethylsilyl)-L-serinate (1.330 g, 2.14 mmole) was dissolved in 4/1/1 THF/methanol/water (4.3 mL). Lithium hydroxide monohydrate (0.138 g, 3.29 mmole) was added and the mixture was stirred at RT for 3 hours. Water (25 mL) was added and the mixture was treated with 1N aq. HCl to pH=4. The mixture was extracted with ethyl acetate (3×10 mL) and the combined organics were dried over magnesium sulfate, filtered and concentrated. The residue was dissolved in DCM (4.3 mL) and L-serine methyl ester hydrochloride (0.434 g, 2.79 mmole) was added followed by N,N-diisopropylethylamine (0.750 mL, 4.31 mmole) and HATU (0.980 g, 2.58 mmole). The mixture was stirred at RT for 18 hours and water (25 mL) was added. The mixture was extracted with DCM (3×10 mL) and the combined organics were dried over magnesium sulfate, filtered and concentrated. The residue was dissolved in DCM (4.3 mL) and triethylamine (0.340 mL, 2.43 mmole) and acetic anhydride (0.230 mL, 2.43 mmole) were added. The mixture was stirred at RT for 18 hours and water (25 mL) was added. The mixture was extracted with DCM (3×10 mL) and the combined organics were dried over magnesium sulfate, filtered and concentrated. The crude residue was purified by silica gel chromatography to provide methyl O-acetyl-N—(N-(2-(4-(((((6-azidohexyl)oxy)carbonyl)amino)methyl)phenyl)thiazole-4-carbonyl)-O-(tert-butyldimethylsilyl)-L-seryl)-L-serinate as a pale yellow oil (0.920 g, 57%). ¹H NMR (400 MHz, CDCl₃): δ 8.26 (d, J=7.1 Hz, 1H), 8.11 (d, J=2.6 Hz, 1H), 7.94 (dd, J=8.3, 1.7 Hz, 2H), 7.47 (d, J=7.8 Hz, 1H), 7.37 (d, J=8.0 Hz, 2H), 5.10 (s, 1H), 4.90 (dq, J=6.9, 3.5 Hz, 1H), 4.66 (td, J=7.2, 3.7 Hz, 1H), 4.49 (ddd, J=10.9, 6.9, 4.0 Hz, 1H), 4.45-4.37 (m, 2H), 4.34 (dd, J=11.4, 3.6 Hz, 1H), 4.23 (dt, J=9.8, 3.5 Hz, 1H), 4.10-4.10 (m, 2H), 3.84-3.79 (m, 1H), 3.77 (s, 3H), 3.27 (t, J=7.0 Hz, 2H), 2.02 (s, 3H), 1.67-1.54 (m, 4H), 1.49-1.32 (m, 4H), 0.96 (s, 9H), 0.17 (s, 3H), 0.15 (s, 3H).

### Methyl 2-(2-(2-(4-(((((6-azidohexyl)oxy)carbonyl)amino)methyl)phenyl)thiazole-4-carboxamido)acrylamido)acrylate (50)

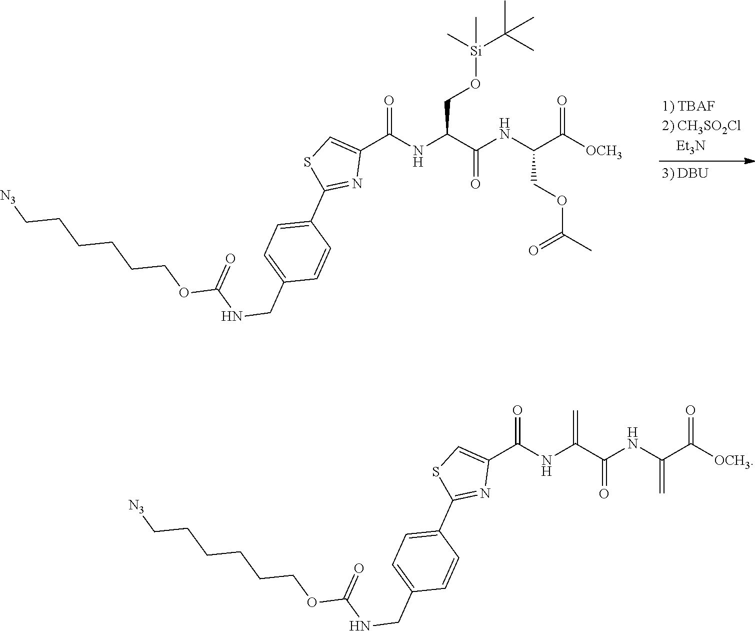 [SMILES: COC(=O)[C@H](COC(C)=O)NC(=O)[C@H](CO[Si](C)(C)C(C)(C)C)NC(=O)c1csc(-c2ccc(CNC(=O)OCCCCCCN=[N+]=[N-])cc2)n1 ; C=C(NC(=O)c1csc(-c2ccc(CNC(=O)OCCCCCCN=[N+]=[N-])cc2)n1)C(=O)NC(=C)C(=O)OC]

Methyl O-acetyl-N—(N-(2-(4-(((((6-azidohexyl)oxy)carbonyl)amino)methyl)phenyl)thiazole-4-carbonyl)-O-(tert-butyldimethylsilyl)-L-seryl)-L-serinate (0.920 g, 1.23 mmole) was dissolved in THE (2.5 mL). TBAF (1M solution in THF, 3.00 mL, 3.00 mmole) was added and the solution was stirred at RT for 2 hours. Water (25 mL) was added and the mixture was extracted with ethyl acetate (3×10 mL). The combined organics were dried over magnesium sulfate, filtered and concentrated. The residue was dissolved in DCM (2.5 mL) and cooled to 0° C. Triethylamine (0.260 mL, 1.45 mmole) and methanesulfonyl chloride (0.145 mL, 1.87 mmole) were added and the mixture was stirred at 0° for 90 minutes. Water (25 mL) was added and the mixture was extracted with DCM (3×10 mL). The combined organics were dried over magnesium sulfate, filtered and concentrated. The residue was dissolved in THE (2.5 mL) and cooled to 0° C. DBU (0.280 mL, 1.87 mmole) was added and the mixture was stirred at 0° for 60 minutes. Water (25 mL) was added and the mixture was extracted with ethyl acetate (3×10 mL). The combined organics were dried over magnesium sulfate, filtered and concentrated. The crude residue was purified by silica gel chromatography to provide methyl 2-(2-(2-(4-(((((6-azidohexyl)oxy)carbonyl)amino)methyl)phenyl)thiazole-4-carboxamido)acrylamido)acrylate as a white solid (0.308 g, 45%). ¹H NMR (400 MHz, CDCl₃): δ 10.01 (s, 1H), 8.55 (s, 1H), 8.13 (s, 1H), 8.03-7.91 (m, 2H), 7.38 (d, J=7.9 Hz, 2H), 6.78 (d, J=2.3 Hz, 1H), 6.71 (s, 1H), 6.03 (d, J=1.3 Hz, 1H), 5.50 (t, J=1.9 Hz, 1H), 5.12 (s, 1H), 4.42 (d, J=6.1 Hz, 2H), 4.11 (td, J=6.9, 1.8 Hz, 2H), 3.90 (s, 3H), 3.27 (t, J=6.8 Hz, 2H), 1.64 (dd, J=14.0, 7.2 Hz, 4H), 1.41 (s, 4H).

### Compound 51: Methyl 2-(2-(2-(4-(2-(tetrahydro-2H-pyran-4-yl)acetamido)piperidin-1-yl)thiazole-4-carboxamido)acrylamido)acrylate

### Methyl O-(tert-butyldimethylsilyl)-N-(2-(4-(2-(tetrahydro-2H-pyran-4-yl)acetamido)piperidin-1-yl)thiazole-4-carbonyl)-L-serinate (51.1)

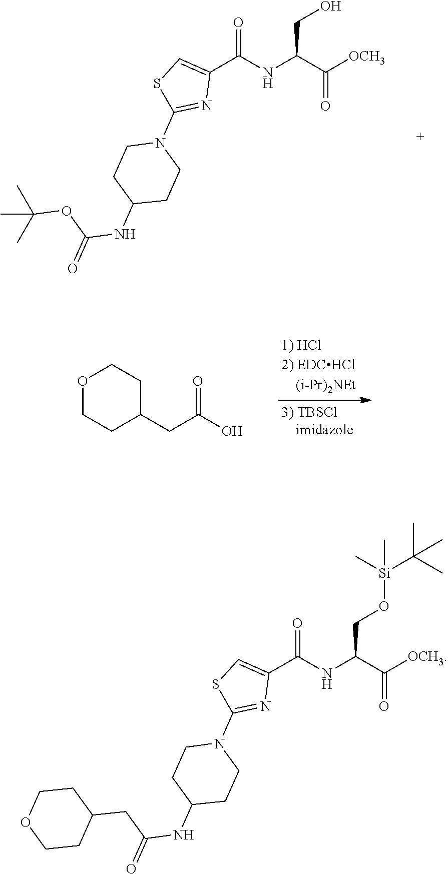 [SMILES: COC(=O)[C@H](CO)NC(=O)c1csc(N2CCC(NC(=O)OC(C)(C)C)CC2)n1 ; O=C(O)CC1CCOCC1 ; COC(=O)[C@H](CO[Si](C)(C)C(C)(C)C)NC(=O)c1csc(N2CCC(NC(=O)CC3CCOCC3)CC2)n1]

Methyl (2-(4-((tert-butoxycarbonyl)amino)piperidin-1-yl)thiazole-4-carbonyl)-L-serinate (Example 46.2, 0.450 g, 1.05 mmole) was dissolved in DCM (2 mL). HCl (4M solution in 1,4-dioxane, 1.00 mL, 4.00 mmole) was added and the mixture was stirred at RT for 3 hours and was concentrated. The residue was suspended in DCM (2 mL) and 2-(tetrahydro-2H-pyran-4-yl)acetic acid (0.227 g, 1.57 mmole) was added. EDC·HCl (0.243 g, 1.27 mmole) and N,N-diisopropylethylamine (0.370 mL, 2.12 mmole) were added and the mixture was stirred at RT for 18 hours. Water (25 mL) was added and the mixture was extracted with DCM (3×10 mL). The combined organics were dried over magnesium sulfate, filtered and concentrated. The residue was suspended in DCM (4 mL) and imidazole (0.085 g, 1.25 mmole) and tert-butyldimethylchlorosilane (0.181 g, 1.20 mmole) were added. The mixture was stirred at RT for 90 minutes and water (25 mL) was added. The mixture was extracted with DCM (3×10 mL) and the combined organics were dried over magnesium sulfate, filtered and concentrated. The crude residue was purified by silica gel chromatography to provide methyl O-(tert-butyldimethylsilyl)-N-(2-(4-(2-(tetrahydro-2H-pyran-4-yl)acetamido)piperidin-1-yl)thiazole-4-carbonyl)-L-serinate as a thick, colorless gel (0.470 g, 79%). ¹H NMR (400 MHz, CDCl₃): δ 7.94 (d, J=8.8 Hz, 1H), 7.38 (s, 1H), 5.39 (d, J=7.9 Hz, 1H), 4.78 (ddd, J=8.8, 3.3, 2.5 Hz, 1H), 4.20-4.13 (m, 1H), 4.08-3.98 (m, 2H), 3.98-3.91 (m, 3H), 3.88 (dd, J=10.0, 3.4 Hz, 1H), 3.76 (s, 3H), 3.41 (td, J=11.8, 2.1 Hz, 2H), 3.15 (dtd, J=13.7, 11.4, 2.8 Hz, 2H), 2.09 (dd, J=2.7, 1.5 Hz, 3H), 2.04-1.97 (m, 2H), 1.68-1.61 (m, 2H), 1.50 (dddd, J=21.5, 13.3, 7.0, 2.8 Hz, 2H), 1.39-1.28 (m, 2H), 0.88 (s, 9H), 0.05 (s, 3H), 0.03 (s, 3H).

### Methyl O-acetyl-N—(O-(tert-butyldimethylsilyl)-N-(2-(4-(2-(tetrahydro-2H-pyran-4-yl)acetamido)piperidin-1-yl)thiazole-4-carbonyl)-L-seryl)-L-serinate (51.2)

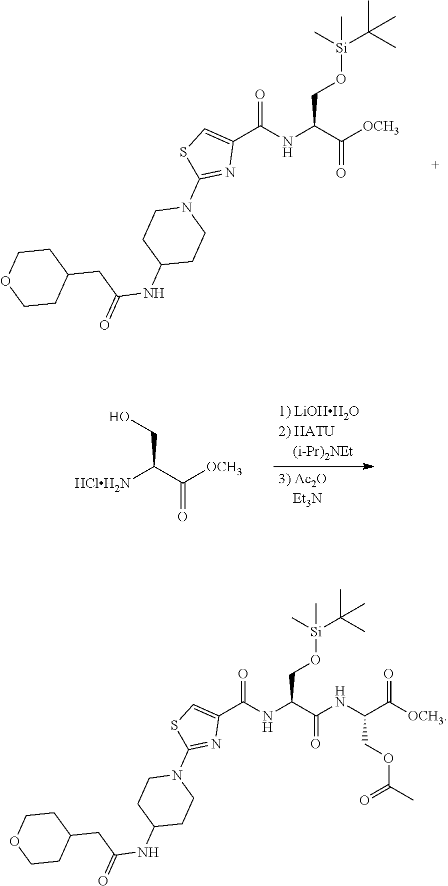

Methyl O-(tert-butyldimethylsilyl)-N-(2-(4-(2-(tetrahydro-2H-pyran-4-yl)acetamido)piperidin-1-yl)thiazole-4-carbonyl)-L-serinate (0.470 g, 0.826 mmole) was dissolved in 4/1/1 THF/methanol/water (1.7 mL). Lithium hydroxide monohydrate (0.070 g, 1.67 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in DCM (1.7 mL). L-Serine methyl ester hydrochloride (0.176 g, 1.13 mmole) was added followed by HATU (0.383 g, 1.01 mmole) and N,N-diisopropylethylamine (0.290 ml, 1.66 mmole). The mixture was stirred at RT for 18 hours and water (25 mL) was added. The mixture was extracted with DCM (3×10 mL) and the combined organics were dried over magnesium sulfate, filtered and concentrated. The residue was dissolved in DCM (1.7 mL). Triethylamine (0.130 mL, 0.928 mmole) and acetic anhydride (0.086 mL, 0.910 mmole) were added and the solution was stirred at RT for 2 hours. Water (25 mL) was added and the mixture was extracted with DCM (3×10 mL). The combined organics were dried over magnesium sulfate, filtered and concentrated. The crude residue was purified by silica gel chromatography to provide methyl O-acetyl-N—(O-(tert-butyldimethylsilyl)-N-(2-(4-(2-(tetrahydro-2H-pyran-4-yl)acetamido)piperidin-1-yl)thiazole-4-carbonyl)-L-seryl)-L-serinate as a colorless gel (0.275 g, 48%). ¹H NMR (400 MHz, CDCl₃): δ 7.97 (d, J=7.1 Hz, 1H), 7.48-7.41 (m, 1H), 7.40 (d, J=2.6 Hz, 1H), 5.41 (d, J=8.0 Hz, 1H), 4.86 (dq, J=8.1, 4.0 Hz, 1H), 4.63-4.55 (m, 1H), 4.51-4.42 (m, 1H), 4.32 (dd, J=11.4, 3.7 Hz, 1H), 4.22-4.16 (m, 1H), 4.07-3.98 (m, 2H), 3.98-3.91 (m, 3H), 3.76 (d, J=2.1 Hz, 3H), 3.75-3.68 (m, 1H), 3.41 (td, J=11.9, 2.1 Hz, 2H), 3.21-3.08 (m, 2H), 2.08 (dd, J=2.6, 1.4 Hz, 3H), 2.02 (d, J=3.4 Hz, 4H), 1.71-1.59 (m, 4H), 1.51 (tq, J=11.6, 6.1, 5.5 Hz, 2H), 1.39-1.29 (m, 2H), 0.92 (d, J=1.0 Hz, 8H), 0.14 (s, 3H), 0.12 (s, 3H).

### Methyl 2-(2-(2-(4-(2-(tetrahydro-2H-pyran-4-yl)acetamido)piperidin-1-yl)thiazole-4-carboxamido)acrylamido)acrylate (51)

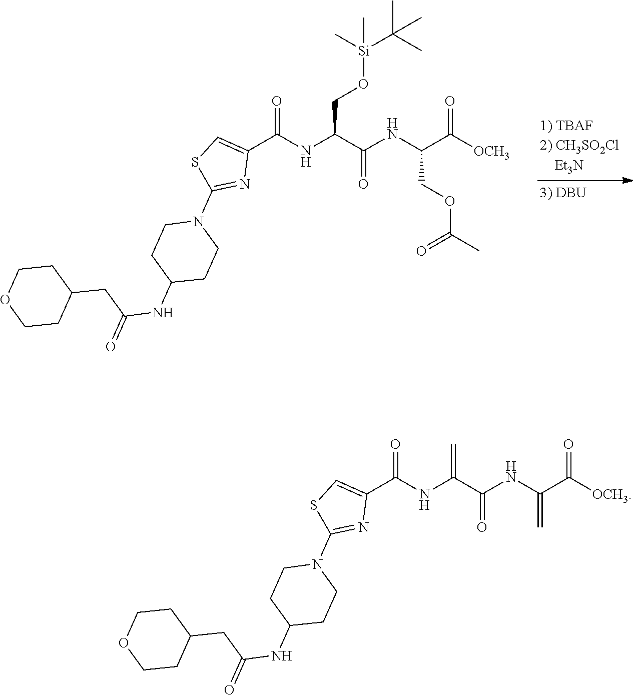 [SMILES: COC(=O)[C@H](COC(C)=O)NC(=O)[C@H](CO[Si](C)(C)C(C)(C)C)NC(=O)c1csc(N2CCC(NC(=O)CC3CCOCC3)CC2)n1 ; C=C(NC(=O)c1csc(N2CCC(NC(=O)CC3CCOCC3)CC2)n1)C(=O)NC(=C)C(=O)OC]

Methyl O-acetyl-N-(0-(tert-butyldimethylsilyl)-N-(2-(4-(2-(tetrahydro-2H-pyran-4-yl)acetamido)piperidin-1l-yl)thiazole-4-carbonyl)-L-seryl)-L-serinate (0.275 g, 0.394 mmole) was dissolved in THF (2 mL). TBAF (1M solution in THF, 0.990 mL, 0.990 mmole) was added and the solution was stirred at RT for 18 hours. Water (25 mL) and sat. aq. sodium chloride (15 mL) were added and the mixture was extracted with ethyl acetate (3×10 mL). The combined organics were dried over magnesium sulfate, filtered and concentrated. The residue was dissolved in DCM (2 mL) and cooled to 0° C. Triethylamine (0.084 mL, 0.599 mmole) and methanesulfonyl chloride (0.046 mL, 0.594 mmole) were added and the mixture was stirred at 0° for 2 hours. Water (25 mL) was added and the mixture was extracted with DCM (3×10 mL). The combined organics were dried over magnesium sulfate, filtered and concentrated. The residue was dissolved in THF (2 mL) and cooled to 0° C. DBU (0.090 mL, 0.602 mmole) was added and the mixture was stirred at 0° for 60 minutes. Water (25 mL) and sat. aq. sodium chloride (15 mL) were added and the mixture was extracted with ethyl acetate (3×10 mL). The combined organics were dried over magnesium sulfate, filtered and concentrated. The crude residue was purified by silica gel chromatography to provide methyl 2-(2-(2-(4-(2-(tetrahydro-2H-pyran-4-yl)acetamido)piperidin-1l-yl)thiazole-4-carboxamido)acrylamido)acrylate as a white solid (0.067 g, 34%). ¹H NMR (400 MHz, CDCl₃): δ 9.71 (s, 1H), 8.51 (s, 1H), 7.43 (s, 1H), 6.72 (d, J=2.1 Hz, 1H), 6.00 (d, J=1.3 Hz, 1H), 5.44 (t, J=1.9 Hz, 1H), 5.3 5 (d, J=7.9 Hz, 1H), 4.10-3.92 (m, 5H), 3.89 (s, 3H), 3.41 (td, J=11.8, 2.1 Hz, 2H), 3.25-3.12 (m, 2H), 2.13-2.00 (m, 5H), 1.64 (d, J=13.6 Hz, 2H), 1.55-1.43 (m, 2H), 1.40-1.22 (m, 2H).

### Compound 52: methyl 2-(2-(2-(4-((tetrahydro-2H-pyran-4-carboxamido)methyl)piperidin-1-yl)thiazole-4-carboxamido)acrylamido)acrylate

### Ethyl 2-(4-(((tert-butoxycarbonyl)amino)methyl)piperidin-1-yl)thiazole-4-carboxylate (52.1)

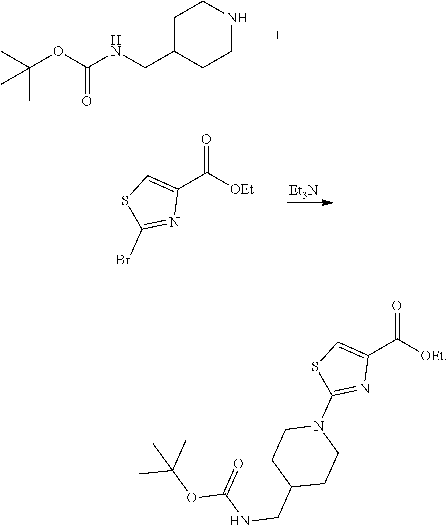 [SMILES: "Et3N" ; CC(C)(C)OC(=O)NCC1CCNCC1 ; CCOC(=O)c1csc(Br)n1 ; CCOC(=O)c1csc(N2CCC(CNC(=O)OC(C)(C)C)CC2)n1]

Tert-butyl (piperidin-4-ylmethyl)carbamate (2.500 g, 11.7 mmole) was dissolved in DMA (12 mL). Ethyl 2-bromothiazole-4-carboxylate (3.027 g, 12.8 mmole) and triethylamine (2.00 mL, 14.3 mmole) were added and the mixture was heated to 80° C. for 20 hours, then was cooled to RT and diluted with ethyl acetate (40 mL). The mixture was washed with water (3×25 mL) and the organics were dried over magnesium sulfate, filtered, and concentrated. The crude residue was purified by silica gel chromatography to provide ethyl 2-(4-(((tert-butoxycarbonyl)amino)methyl)piperidin-1-yl)thiazole-4-carboxylate as a pale yellow oil (4.194 g). ¹H NMR (400 MHz, CDCl₃): δ 7.42 (s, 1H), 4.69 (d, J=6.4 Hz, 1H), 4.35 (q, J=7.1 Hz, 2H), 4.10-4.01 (m, 2H), 3.09-2.94 (m, 4H), 1.86-1.76 (m, 2H), 1.45 (s, 9H), 1.37 (t, J=7.1 Hz, 3H), 1.34-1.28 (m, 2H).

### Methyl (2-(4-(((tert-butoxycarbonyl)amino)methyl)piperidin-1-yl)thiazole-4-carbonyl)-L-serinate (52.2)

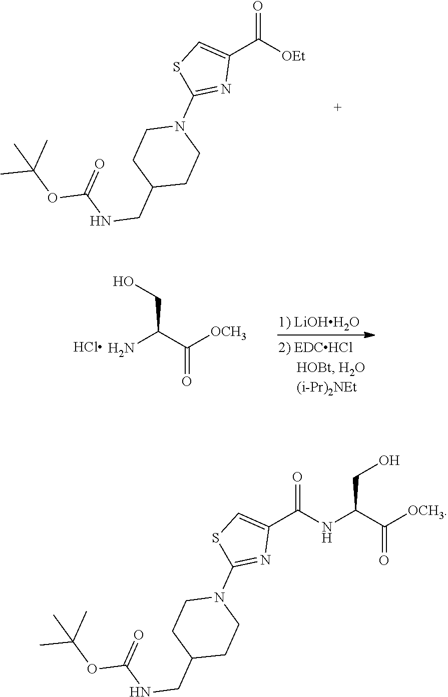 [SMILES: CCOC(=O)c1csc(N2CCC(CNC(=O)OC(C)(C)C)CC2)n1 ; COC(=O)[C@@H](N)CO ; COC(=O)[C@H](CO)NC(=O)c1csc(N2CCC(CNC(=O)OC(C)(C)C)CC2)n1]

Ethyl 2-(4-(((tert-butoxycarbonyl)amino)methyl)piperidin-1-yl)thiazole-4-carboxylate (4.194 g, 11.4 mmole) was dissolved in 4/1/1 THF/methanol/water (22 mL). Lithium hydroxide monohydrate (0.715 g, 17.0 mmole) was added and the mixture was stirred at RT for 3 hours. Water (50 mL) was added and the solution was treated with 1N aq. HCl to pH=4. The mixture was extracted with ethyl acetate (3×25 mL) and the combined organics were dried over magnesium sulfate, filtered, and concentrated. The residue was dissolved in DCM (22 mL). L-Serine methyl ester hydrochloride (2.128 g, 13.7 mmole) was added followed by N,N-diisopropylethylamine (4.00 mL, 23.0 mmole), HOBt·H₂O (2.106 g, 13.8 mmole), and EDC·HCl (2.617 g, 13.7 mmole). The mixture was stirred at RT for 2 ½ days and water (50 mL) was added. The two layers were separated and the aqueous layer was extracted with DCM (2×10 mL). The combined organics were dried over magnesium sulfate, filtered, and concentrated and the crude residue was purified by silica gel chromatography to provide methyl (2-(4-(((tert-butoxycarbonyl)amino)methyl)piperidin-1-yl)thiazole-4-carbonyl)-L-serinate as a white solid (5.150 g, 99%). ¹H NMR (400 MHz, CDCl₃): δ 8.01 (d, J=7.4 Hz, 1H), 7.38 (s, 1H), 4.81 (dt, J =7.5, 3.8 Hz, 1H), 4.71 (s, 1H), 4.09-3.97 (m, 4H), 3.83 (s, 3H), 3.12-2.98 (m, 4H), 2.95 (t, J=6.2 Hz, 1H), 1.88-1.78 (m, 2H), 1.47 (s, 9H), 1.40-1.29 (m, 2H).

### Methyl O-(tert-butyldimethylsilyl)-N-(2-(4-((tetrahydro-2H-pyran-4-carboxamido)methyl)piperidin-1-yl)thiazole-4-carbonyl)-L-serinate (52.3)

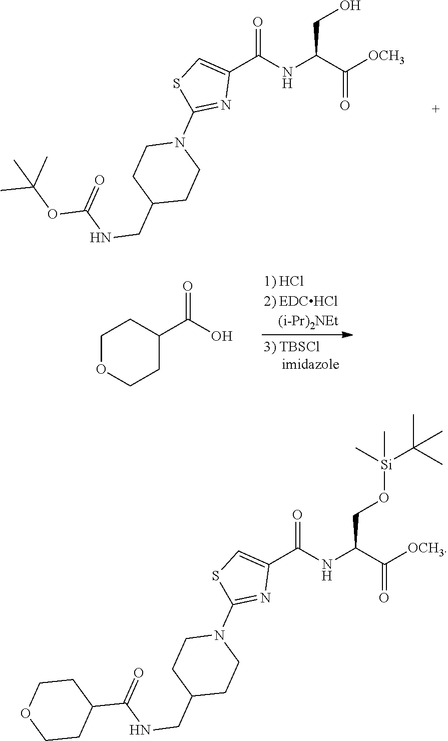 [SMILES: COC(=O)[C@H](CO)NC(=O)c1csc(N2CCC(CNC(=O)OC(C)(C)C)CC2)n1 ; O=C(O)C1CCOCC1 ; COC(=O)[C@H](CO[Si](C)(C)C(C)(C)C)NC(=O)c1csc(N2CCC(CNC(=O)C3CCOCC3)CC2)n1]

Methyl (2-(4-(((tert-butoxycarbonyl)amino)methyl)piperidin-1-yl)thiazole-4-carbonyl)-L-serinate (0.601 g, 1.36 mmole) was dissolved in DCM (2.8 mL). HCl (4M solution in 1,4-dioxane, 1.40 mL, 5.60 mmole) was added and the mixture was stirred at RT for 3 hours, then was concentrated. Tetrahydro-2H-pyran-4-carboxylic acid (0.256 g, 1.97 mmole) was added followed by EDC·HCl (0.333 g, 1.74 mmole) and N,N-diisopropylethylamine (0.500 mL, 2.87 mmole) and the mixture was stirred at RT for 18 hours. Water (25 mL) was added and the mixture was extracted with DCM (3×10 mL). The combined organics were dried over magnesium sulfate, filtered and concentrated. The residue was dissolved in DCM (2.8 mL). Imidazole (0.108 g, 1.59 mmole) and tert-butyldimethylchlorosilane (0.29 g, 1.59 mmole) were added and the solution was stirred at RT for 2 hours. Water (25 mL) was added and the mixture was extracted with DCM (3×10 mL). The combined organics were dried over magnesium sulfate, filtered and concentrated. The crude residue was purified by silica gel chromatography to provide methyl O-(tert-butyldimethylsilyl)-N-(2-(4-((tetrahydro-2H-pyran-4-carboxamido)methyl)piperidin-1-yl)thiazole-4-carbonyl)-L-serinate as a colorless oil (0.570 g, 74%). ¹H NMR (400 MHz, CDCl₃): δ 7.96 (d, J=8.8 Hz, 1H), 7.36 (s, 1H), 5.72 (t, J=6.1 Hz, 1H), 4.82-4.73 (m, 1H), 4.19-4.15 (m, 1H), 4.06-3.93 (m, 4H), 3.88 (dd, J=10.0, 3.4 Hz, 1H), 3.76 (s, 3H), 3.42 (td, J=11.5, 2.9 Hz, 2H), 3.21 (t, J=6.2 Hz, 2H), 3.05-2.91 (m, 2H), 2.36 (tt, J=11.1, 4.4 Hz, 1H), 1.90-1.71 (m, 8H), 1.37-1.28 (m, 1H), 0.88 (s, 9H), 0.05 (s, 3H), 0.03 (s, 3H).

### Methyl O-acetyl-N—(O-(tert-butyldimethylsilyl)-N-(2-(4-((tetrahydro-2H-pyran-4-carboxamido)methyl)piperidin-1-yl)thiazole-4-carbonyl)-L-seryl)-L-serinate (52.4)

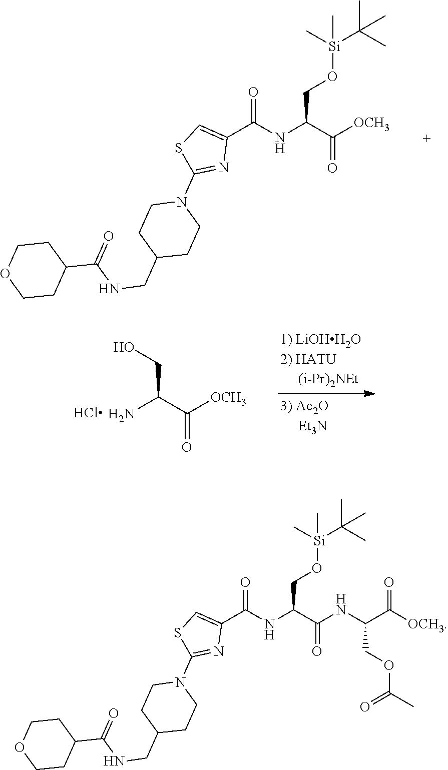 [SMILES: COC(=O)[C@H](CO[Si](C)(C)C(C)(C)C)NC(=O)c1csc(N2CCC(CNC(=O)C3CCOCC3)CC2)n1 ; COC(=O)[C@@H](N)CO ; COC(=O)[C@H](COC(C)=O)NC(=O)[C@H](CO[Si](C)(C)C(C)(C)C)NC(=O)c1csc(N2CCC(CNC(=O)C3CCOCC3)CC2)n1]

Methyl O-(tert-butyldimethylsilyl)-N-(2-(4-((tetrahydro-2H-pyran-4-carboxamido)methyl)piperidin-1-yl)thiazole-4-carbonyl)-L-serinate (0.570 g, 1.00 mmole) was dissolved in 4/1/1 THF/methanol/water (2 mL). Lithium hydroxide monohydrate (0.082 g, 1.95 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in DCM (2 mL). L-Serine methyl ester hydrochloride (0.205 g, 1.32 mmole) was added followed by HATU (0.474 g, 1.25 mmole) and N,N-diisopropylethylamine (0.350 mL, 2.01 mmole). The mixture was stirred at RT for 18 hours and water (25 mL) was added. The mixture was extracted with DCM (3×10 mL) and the combined organics were dried over magnesium sulfate, filtered and concentrated. The residue was dissolved in DCM (2 mL). Triethylamine (0.160 mL, 1.14 mmole) and acetic anhydride (0.105 mL, 0.952 mmole) were added and the solution was stirred at RT for 2 hours. Water (25 mL) was added and the mixture was extracted with DCM (3×10 mL). The combined organics were dried over magnesium sulfate, filtered and concentrated. The crude residue was purified by silica gel chromatography to provide methyl O-acetyl-N—(O-(tert-butyldimethylsilyl)-N-(2-(4-((tetrahydro-2H-pyran-4-carboxamido)methyl)piperidin-1-yl)thiazole-4-carbonyl)-L-seryl)-L-serinate as a white solid (0.509 g, 73%). ¹H NMR (400 MHz, CDCl₃): δ 7.99 (d, J=7.0 Hz, 1H), 7.44 (d, J=7.8 Hz, 1H), 7.41-7.36 (m, 1H), 5.65 (t, J=6.1 Hz, 1H), 4.87 (dq, J=8.3, 4.1 Hz, 1H), 4.58 (td, J=7.2, 3.7 Hz, 1H), 4.51-4.43 (m, 1H), 4.31 (dd, J=11.4, 3.7 Hz, 1H), 4.22-4.15 (m, 1H), 4.02 (ddt, J=11.6, 8.7, 3.6 Hz, 5H), 3.76 (d, J=2.1 Hz, 4H), 3.74-3.70 (m, 1H), 3.42 (td, J=11.4, 2.9 Hz, 2H), 3.20 (q, J=7.6, 6.9 Hz, 3H), 3.05-2.94 (m, 3H), 2.36 (tt, J=11.0, 4.5 Hz, 1H), 2.02 (d, J=2.7 Hz, 3H), 1.87-1.73 (m, 8H), 1.42 (s, 3H), 1.39-1.28 (m, 2H), 0.92 (s, 9H), 0.14 (s, 3H), 0.12 (s, 3H).

### Methyl 2-(2-(2-(4-((tetrahydro-2H-pyran-4-carboxamido)methyl)piperidin-1-yl)thiazole-4-carboxamido)acrylamido)acrylate (52)

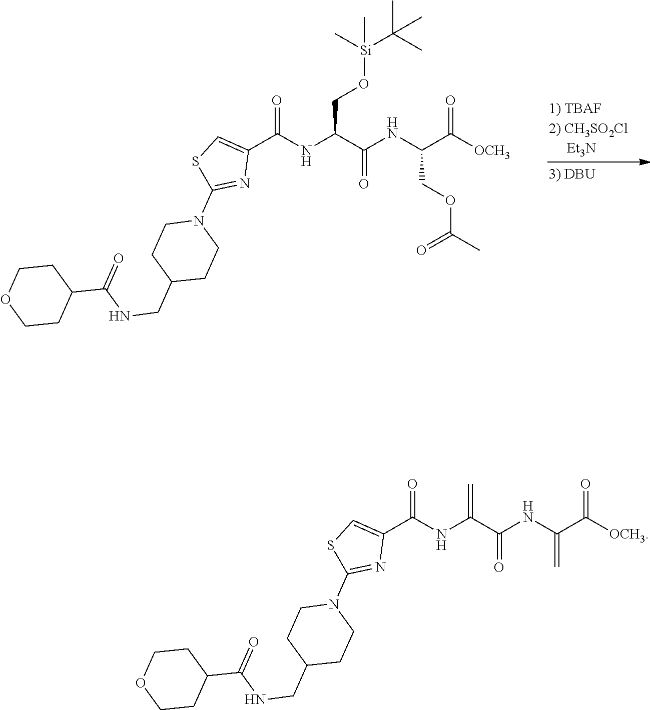 [SMILES: COC(=O)[C@H](COC(C)=O)NC(=O)[C@H](CO[Si](C)(C)C(C)(C)C)NC(=O)c1csc(N2CCC(CNC(=O)C3CCOCC3)CC2)n1 ; C=C(NC(=O)c1csc(N2CCC(CNC(=O)C3CCOCC3)CC2)n1)C(=O)NC(=C)C(=O)OC]

Methyl O-acetyl-N—(O-(tert-butyldimethlysilyl)-N-(2-(4-((tetrahydro-2H-pyran-4-carboxamido)methyl)piperdin-1-yl)thiazole-4-carbonyl)-L-seryl)-L-serinate (0.509 g, 0.729 mmole) was dissolved in THF (2 mL). TBAF (1M solution in THF, 1.80 mL, 1.80 mmole) was added and the solution was stirred at RT for 18 hours. Water (25 mL) and sat. aq. sodium chloride (15 mL) were added and the mixture was extracted with ethyl acetate (3×10 mL). The combined organics were dried over magnesium sulfate, filtered and concentrated. The residue was dissolved in DCM (2 mL) and cooled to 0° C. Triethylamine (0.160 mL, 1.14 mmole) and methanesulfonyl chloride (0.086 mL, 1.11 mmole) were added and the mixture was stirred at 0° for 2 hours. Water (25 mL) was added and the mixture was extracted with DCM (3×10 mL). The combined organics were dried over magnesium sulfate, filtered and concentrated. The residue was dissolved in THE (2 mL) and cooled to 0° C. DBU (0.170 mL, 1.14 mmole) was added and the mixture was stirred at 0° for 60 minutes. Water (25 mL) and sat. aq. sodium chloride (15 mL) were added and the mixture was extracted with ethyl acetate (3×10 mL). The combined organics were dried over magnesium sulfate, filtered and concentrated. The crude residue was purified by silica gel chromatography to provide methyl 2-(2-(2-(4-((tetrahydro-2H-pyran-4-carboxamido)methyl)piperidin-1-yl)thiazole-4-carboxamido)acrylamido)acrylate as a white solid (0.122 g, 33%). ¹H NMR (400 MHz, CDCl₃): δ 9.73 (s, 1H), 8.51 (s, 1H), 7.41 (s, 1H), 6.72 (d, J=2.1 Hz, 1H), 6.65 (s, 1H), 6.00 (d, J=1.3 Hz, 1H), 5.64-5.53 (m, 1H), 5.43 (t, J=1.9 Hz, 1H), 4.10-3.97 (m, 4H), 3.89 (s, 3H), 3.42 (td, J=11.4, 3.0 Hz, 2H), 3.22 (t, J=6.2 Hz, 2H), 3.02 (td, J=12.7, 2.6 Hz, 2H), 2.35 (tt, J=10.9, 4.6 Hz, 1H), 1.89-1.70 (m, 7H), 1.41-1.28 (m, 2H).

### Compound 53: Methyl 2-(2-(2-(4-((tetrahydro-2H-pyran-4-yl)carbamoyl)piperidin-1-yl)thiazole-4-carboxamido)acrylamido)acrylate

### 1-Benzyl 4-(tert-butyl) piperidine-1,4-dicarboxylate (53.1)

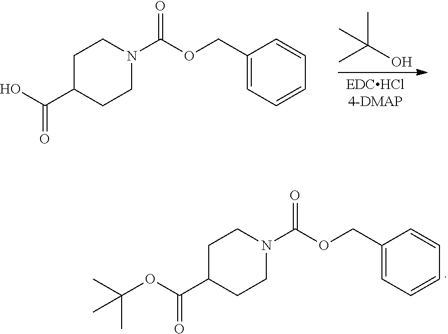

1-((Benzyloxy)carbonyl)piperidine-4-carboxylic acid (3.014 g, 11.4 mmole) was dissolved in DCM (22 mL) and cooled in an ice/salt bath. Tert-butanol (2.80 mL, 29.3 mmole) and 4-DMAP (0.695 g, 5.69 mmole) were added followed by EDC·HCl (2.185 g, 11.4 mmole). The mixture was stirred at 0° C. for 2 hours, then was warmed to RT and stirred for 18 hours. 1N aq. HCl (50 mL) was added and the two layers were separated. The aqueous layer was extracted with DCM (2×15 mL) and the combined organics were dried over magnesium sulfate, filtered and concentrated. The crude residue was purified by silica gel chromatography to provide 1-benzyl 4-(tert-butyl) piperidine-1,4-dicarboxylate as a colorless oil (2.997 g, 82%). ¹H NMR (400 MHz, CDCl₃): δ 7.41-7.29 (m, 5H), 5.12 (s, 2H), 4.11-4.00 (m, 2H), 2.92 (t, J=12.1 Hz, 2H), 2.36 (tt, J=10.8, 3.9 Hz, 1H), 1.86 (d, J=13.4 Hz, 2H), 1.66-1.55 (m, 2H), 1.44 (s, 9H).

### Ethyl 2-(4-(tert-butoxycarbonyl)piperidin-1-yl)thiazole-4-carboxylate (53.2)

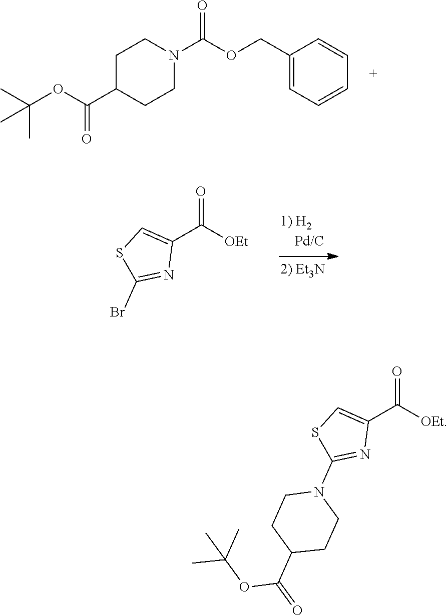 [SMILES: CC(C)(C)OC(=O)C1CCN(C(=O)OCc2ccccc2)CC1 ; CCOC(=O)c1csc(Br)n1 ; CCOC(=O)c1csc(N2CCC(C(=O)OC(C)(C)C)CC2)n1]

A clean, dry flask was charged with 10% palladium on carbon (0.500 g, 0.470 mmole). Enough methanol was added dropwise to wet the catalyst. 1-Benzyl 4-(tert-butyl) piperidine-1,4-dicarboxylate (2.997 g, 9.38 mmole) was dissolved in the remaining methanol (18 mL) and added. The atmosphere was changed to hydrogen (balloon) and the mixture was vigorously stirred at RT for 2 hours. The mixture was filtered through a pad of Celite and the filter cake was washed with methanol. The combined filtrates were concentrated. The residue was dissolved in DMA (10 mL) and ethyl 2-bromothiazole-4-carboxylate (2.224 g, 9.42 mmole) and triethylamine (1.60 mL, 11.4 mmole) were added. The mixture was heated to 80° C. for 18 hours and was cooled to RT. The mixture was diluted with ethyl acetate (50 mL) and washed with water (3×25 mL). The organics were dried over magnesium sulfate, filtered and concentrated. The crude residue was purified by silica gel chromatography to provide ethyl 2-(4-(tert-butoxycarbonyl)piperi din-1l-yl)thiazole-4-carboxylate as a yellow solid (2.340 g, 73%). ¹H NMR (400 MHz, CDCl₃): δ 7.44 (s, 1H), 4.35 (q, J=7.0 Hz, 2H), 3.97 (dt, J=13.0, 3.7 Hz, 2H), 3.21-3.07 (m, 2H), 2.43 (tt, J=10.7, 3.0 Hz, 1H), 2.03-1.92 (m, 2H), 1.87-1.72 (m, 2H), 1.46 (s, 9H), 1.37 (t, J=7.1 Hz, 3H)

### Tert-butyl (S)-1-(4-((3-hydroxy-1-methoxy-1-oxopropan-2-yl)carbamoyl)thiazol-2-yl)piperidine-4-carboxylate (53.3)

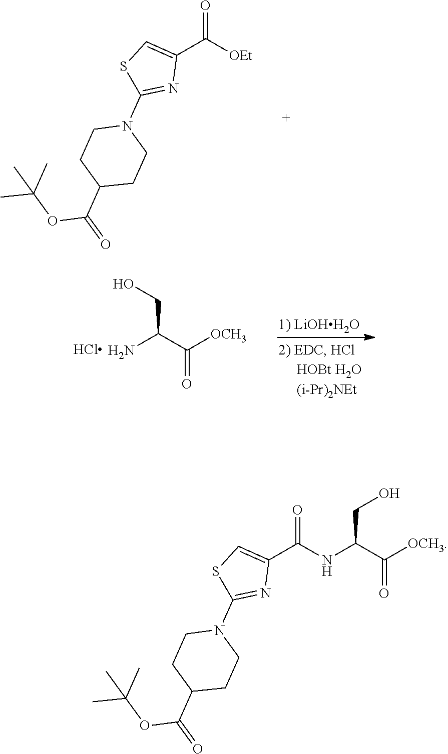

Ethyl 2-(4-(tert-butoxycarbonyl)piperidin-1-yl)thiazole-4-carboxylate (2.340 g, 6.87 mmole) was dissolved in 4/1/1 THF/methanol/water (14 mL). Lithium hydroxide monohydrate (0.434 g, 10.3 mmole) was added and the mixture was stirred at RT for 90 minutes. Water (25 mL) was added and the solution was treated with 1N aq. HCl to pH=4. The mixture was extracted with ethyl acetate (3×15 mL) and the combined organics were dried over magnesium sulfate, filtered and concentrated. The residue was dissolved in DCM (14 mL) and L-serine methyl ester hydrochloride (1.429 g, 9.18 mmole) was added. N,N-Diisopropylethylamine (2.40 mL, 13.8 mmole) and EDC·HCl (1.586, 8.27 mmole) were added and the mixture was stirred at RT for 18 hours. Water (25 mL) and 1N aq. HCl (25 mL) were added and the two layers were separated. The aqueous layer was extracted with DCM (2×15 mL) and the combined organics were dried over magnesium sulfate, filtered and concentrated. The crude residue was purified by silica gel chromatography to provide tert-butyl (5)-1-(4-((3-hydroxy-1-methoxy-1-oxopropan-2-yl)carbamoyl)thiazol-2-yl)piperidine-4-carboxylate as a white solid (1.783 g, 63%). ¹H NMR (400 MHz, CDCl₃): δ 8.00 (d, J=7.4 Hz, 1H), 7.38 (s, 1H), 4.79 (dt, J=7.6, 3.9 Hz, 1H), 4.04 (d, J=3.9 Hz, 2H), 3.97-3.87 (m, 2H), 3.81 (s, 3H), 3.13 (ddd, J=13.1, 11.1, 3.2 Hz, 2H), 3.00 (br s, 1H), 2.44 (tt, J=10.6, 3.8 Hz, 1H), 2.03-1.95 (m, 2H), 1.87-1.72 (m, 2H), 1.46 (s, 9H).

### Methyl O-(tert-butyldimethylsilyl)-N-(2-(4-((tetrahydro-2H-pyran-4-yl)carbamoyl)piperidin-1-yl)thiazole-4-carbonyl)-L-serinate (53.4)

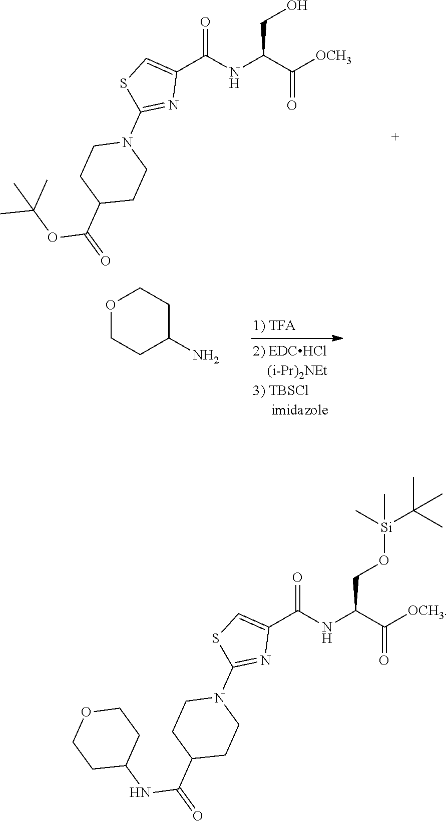

Tert-butyl (S)-1-(4-((3-hydroxy-1-methoxy-1-oxopropan-2-yl)carbamoyl)thiazol-2-yl)piperidine-4-carboxylate (0.753 g, 1.82 mmole) was dissolved in DCM (4 mL). Trifluoroacetic acid (2.00 mL, 26.0 mmole) was added and the mixture was stirred at RT for 24 hours, then was concentrated. The residue was suspended in toluene (5 mL) and concentrated. DCM (4 mL) and N,N-diisopropylethylamine (0.640 mL, 3.67 mmole) were added. Tetrahydro-2H-pyran-4-amine (0.250 mL, 2.41 mmole) and EDC·HCl (0.421 g, 2.20 mmole) were added and the mixture was stirred at RT for 18 hours and water (25 mL) was added. The mixture was extracted with DCM (3×10 mL) and the combined organics were dried over magnesium sulfate, filtered and concentrated. The residue was dissolved in DCM (4 mL) and imidazole (0.139 g, 2.04 mmole) and tert-butyldimethylchlorosilane (0.304 g, 2.02 mmole) were added. The mixture was stirred at RT for 18 hours and water (25 mL) was added. The mixture was extracted with DCM (3×10 mL) and the combined organics were dried over magnesium sulfate, filtered and concentrated. The crude residue was purified by silica gel chromatography to provide methyl O-(tert-butyldimethylsilyl)-N-(2-(4-((tetrahydro-2H-pyran-4-yl)carbamoyl)piperidin-1-yl)thiazole-4-carbonyl)-L-serinate as a thick, pale yellow oil (0.300 g, 30%). ¹H NMR (400 MHz, CDCl₃): δ 7.96 (d, J=8.8 Hz, 1H), 7.38 (s, 1H), 5.39 (d, J=7.9 Hz, 1H), 4.83-4.74 (m, 1H), 4.16 (dd, J=10.0, 2.5 Hz, 1H), 4.08-3.92 (m, 5H), 3.92-3.85 (m, 1H), 3.76 (s, 3H), 3.48 (td, J=11.8, 2.1 Hz, 2H), 3.05 (dddd, J=12.8, 11.4, 4.9, 3.4 Hz, 2H), 2.29 (tt, J=11.3, 3.9 Hz, 1H), 1.97-1.79 (m, 6H), 1.46 (dtd, J=12.9, 11.4, 4.5 Hz, 2H), 0.88 (s, 9H), 0.05 (s, 3H), 0.03 (s, 3H).

### Methyl O-acetyl-N-(0-(tert-butyldimethylsilyl)-N-(2-(4-((tetrahydro-2H-pyran-4-yl)carbamoyl)piperidin-1-yl)thiazole-4-carbonyl)-L-seryl)-L-serinate (53.5)

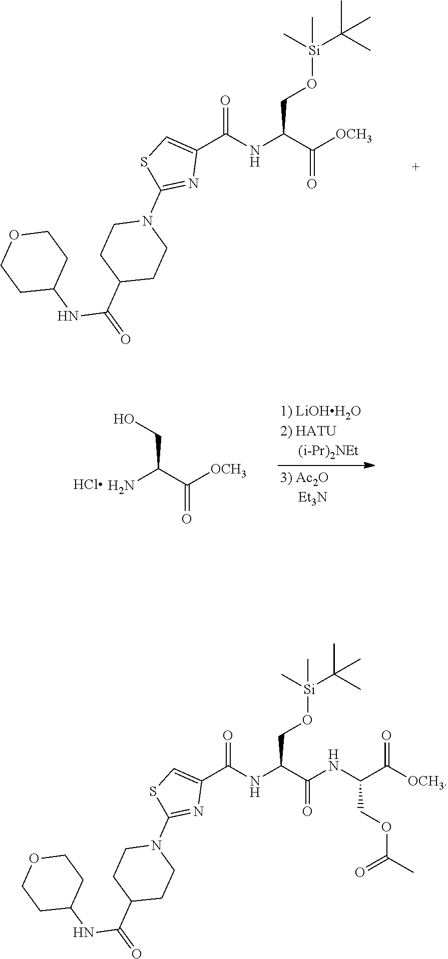 [SMILES: COC(=O)[C@H](CO[Si](C)(C)C(C)(C)C)NC(=O)c1csc(N2CCC(C(=O)NC3CCOCC3)CC2)n1 ; COC(=O)[C@@H](N)CO ; COC(=O)[C@H](COC(C)=O)NC(=O)[C@H](CO[Si](C)(C)C(C)(C)C)NC(=O)c1csc(N2CCC(C(=O)NC3CCOCC3)CC2)n1]

Methyl O-(tert-butyldimethylsilyl)-N-(2-(4-((tetrahydro-2H-pyran-4-yl)carbamoyl)piperidin-1-yl)thiazole-4-carbonyl)-L-serinate (0.300 g, 0.541 mmole) was dissolved in 4/1/1 THF/methanol/water (2 mL). Lithium hydroxide monohydrate (0.045 g, 1.07 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in DCM (2 mL). L-Serine methyl ester hydrochloride (0.117 g, 0.752 mmole) was added followed by HATU (0.245 g, 0.644 mmole) and N,N-diisopropylethylamine (0.190 mL, 1.09 mmole). The mixture was stirred at RT for 18 hours and water (25 mL) was added. The mixture was extracted with DCM (3×10 mL) and the combined organics were dried over magnesium sulfate, filtered and concentrated. The residue was dissolved in DCM (2 mL). Triethylamine (0.084 mL, 0.599 mmole) and acetic anhydride (0.057 mL, 0.603 mmole) were added and the solution was stirred at RT for 18 hours. Water (25 mL) was added and the mixture was extracted with DCM (3×10 mL). The combined organics were dried over magnesium sulfate, filtered and concentrated. The crude residue was purified by silica gel chromatography to provide methyl O-acetyl-N—(O-(tert-butyldimethylsilyl)-N-(2-(4-((tetrahydro-2H-pyran-4-yl)carbamoyl)piperidin-1-yl)thiazole-4-carbonyl)-L-seryl)-L-serinate as a thick, colorless gel (0.227 g, 61%). ¹H NMR (400 MHz, CDCl₃): δ 7.96 (d, J=7.1 Hz, 1H), 7.45-7.39 (m, 1H), 7.37 (d, J=2.7 Hz, 1H), 5.34 (d, J=7.9 Hz, 1H), 4.84 (dq, J=8.4, 4.2 Hz, 1H), 4.56 (ddd, J=7.3, 6.1, 3.6 Hz, 1H), 4.50-4.39 (m, 1H), 4.29 (dd, J=11.4, 3.7 Hz, 1H), 4.20-4.12 (m, 1H), 4.07-3.89 (m, 5H), 3.74 (d, J=2.5 Hz, 3H), 3.72-3.65 (m, 1H), 3.46 (td, J=11.8, 2.2 Hz, 2H), 3.08-2.98 (m, 2H), 2.26 (tt, J=11.2, 3.9 Hz, 1H), 2.00 (s, 3H), 1.95-1.75 (m, 6H), 1.51-1.36 (m, 2H), 0.90 (s, 9H), 0.12 (s, 3H), 0.11 (s, 3H).

### Methyl 2-(2-(2-(4-((tetrahydro-2H-pyran-4-yl)carbamoyl)piperidin-1-yl)thiazole-4-carboxamido)acrylamido)acrylate (53)

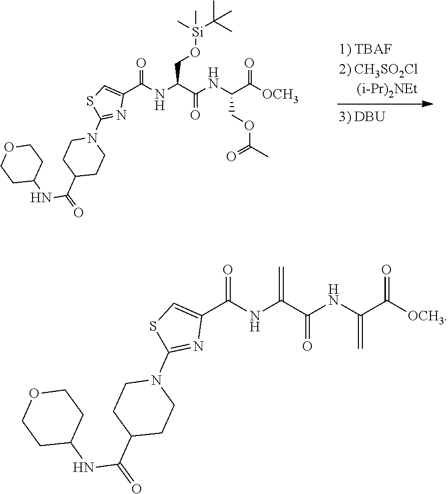 [SMILES: COC(=O)[C@H](COC(C)=O)NC(=O)[C@H](CO[Si](C)(C)C(C)(C)C)NC(=O)c1csc(N2CCC(C(=O)NC3CCOCC3)CC2)n1 ; C=C(NC(=O)c1csc(N2CCC(C(=O)NC3CCOCC3)CC2)n1)C(=O)NC(=C)C(=O)OC]

Methyl O-acetyl-N—(O-(tert-butyldimethylsilyl)-N-(2-(4-((tetrahydro-2H-pyran-4-yl)carbamoyl)piperidin-1l-yl)thiazole-4-carbonyl)-L-seryl)-L-serinate (0.227 g, 0.332 mmole) was dissolved in THE (2 mL). TBAF (1M solution in THF, 0.830 mL, 0.830 mmole) was added and the solution was stirred at RT for 18 hours. Water (25 mL) was added and the mixture was extracted with ethyl acetate (3×10 mL). The combined organics were dried over magnesium sulfate, filtered and concentrated. The residue was dissolved in DCM (2 mL) and cooled to 0° C. Triethylamine (0.070 mL, 0.499 mmole) and methanesulfonyl chloride (0.039 mL, 0.504 mmole) were added and the mixture was stirred at 0° for 90 minutes. Water (25 mL) was added and the mixture was extracted with DCM (3×10 mL). The combined organics were dried over magnesium sulfate, filtered and concentrated. The residue was dissolved in THE (2 mL) and cooled to 0° C. DBU (0.080 mL, 0.535 mmole) was added and the mixture was stirred at 0° for 90 minutes. Water (25 mL) was added and the mixture was extracted with ethyl acetate (3×10 mL). The combined organics were dried over magnesium sulfate, filtered and concentrated. The crude residue was purified by silica gel chromatography to provide methyl 2-(2-(2-(4-((tetrahydro-2H-pyran-4-yl)carbamoyl)piperidin-1-yl)thiazole-4-carboxamido)acrylamido)acrylate as a white solid (0.074 g, 45%). ¹H NMR (400 MHz, CDCl₃): δ 9.72 (s, 1H), 8.51 (s, 1H), 7.43 (s, 1H), 6.72 (d, J=2.1 Hz, 1H), 6.65 (s, 1H), 6.00 (d, J=1.3 Hz, 1H), 5.44 (t, J=1.9 Hz, 1H), 5.40 (d, J=8.0 Hz, 1H), 4.10-4.04 (m, 2H), 4.04-3.92 (m, 3H), 3.89 (s, 3H), 3.48 (td, J=11.8, 2.1 Hz, 2H), 3.08 (ddd, J=13.0, 11.6, 3.2 Hz, 2H), 2.29 (tt, J=11.3, 3.8 Hz, 1H), 2.00-1.78 (m, 6H), 1.46 (dtd, J=12.9, 11.4, 4.5 Hz, 2H).

### Compound 54: methyl (S)-2-(2-(2-(3-(tetrahydro-2H-pyran-4-carboxamido)piperidin-1-yl)thiazole-4-carboxamido)acrylamido)acrylate

### Ethyl (S)-2-(3-((tert-butoxycarbonyl)amino)piperidin-1-yl)thiazole-4-carboxylate (54.1)

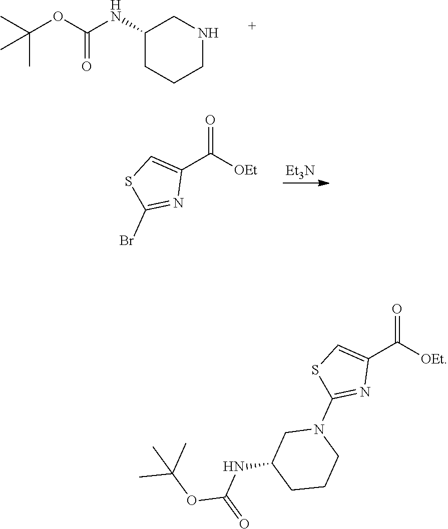 [SMILES: "Et3N" ; CC(C)(C)OC(=O)N[C@H]1CCCNC1 ; CCOC(=O)c1csc(Br)n1 ; CCOC(=O)c1csc(N2CCC[C@H](NC(=O)OC(C)(C)C)C2)n1]

Tert-butyl (S)-piperidin-3-ylcarbamate (2.033 g, 10.2 mmole) was dissolved in DMA (10 mL). Ethyl 2-bromothiazole-4-carboxylate (2.398 g, 10.2 mmole) and triethylamine (1.70 mL, 12.1 mmole) were added and the mixture was heated to 80° C. for 18 hours. The mixture was cooled to RT and diluted with ethyl acetate (50 mL). The mixture was washed with water (3×25 mL) and the organics were dried over magnesium sulfate, filtered and concentrated. The crude residue was purified by silica gel chromatography to provide ethyl (S)-2-(3-((tert-butoxycarbonyl)amino)piperidin-1-yl)thiazole-4-carboxylate as an orange solid (3.087 g, 85%). ¹H NMR (400 MHz, CDCl₃): δ 7.44 (s, 1H), 4.70 (s, 1H), 4.35 (q, J=7.1 Hz, 2H), 3.80 (s, 1H), 3.70 (d, J=12.5 Hz, 1H), 3.61 (s, 1H), 3.46 (s, 1H), 3.29 (t, J=9.9 Hz, 1H), 1.90 (qd, J=7.5, 4.0 Hz, 1H), 1.82 (dtt, J=10.9, 7.3, 3.5 Hz, 1H), 1.75-1.64 (m, 1H), 1.60 (s, 1H), 1.45 (s, 9H), 1.37 (t, J=7.1 Hz, 3H).

### Methyl (2-((S)-3-((tert-butoxycarbonyl)amino)piperidin-1-yl)thiazole-4-carbonyl)-L-serinate (54.2)

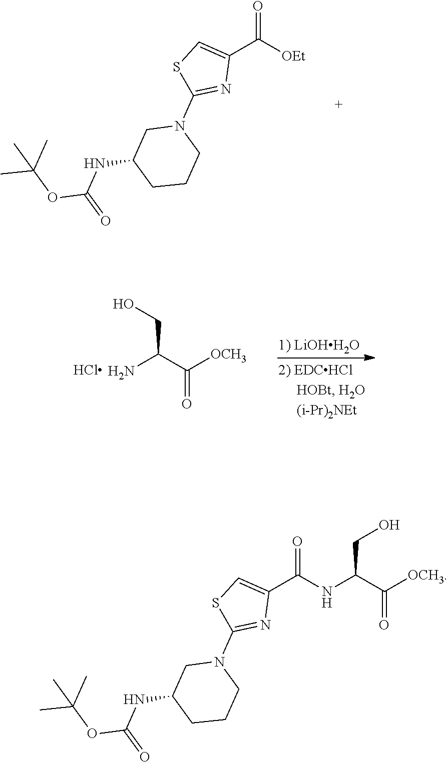 [SMILES: CCOC(=O)c1csc(N2CCC[C@H](NC(=O)OC(C)(C)C)C2)n1 ; COC(=O)[C@@H](N)CO ; COC(=O)[C@H](CO)NC(=O)c1csc(N2CCC[C@H](NC(=O)OC(C)(C)C)C2)n1]

Ethyl (S)-2-(3-((tert-butoxycarbonyl)amino)piperidin-1-yl)thiazole-4-carboxylate (3.087 g, 8.68 mmole) was dissolved in 4/1/1 THF/methanol/water (15 mL). Lithium hydroxide monohydrate (0.545 g, 13.0 mmole) was added and the mixture was stirred at RT for 2 hours. Water (75 mL) was added and the solution was treated with 1N aq. HCl to pH=4. The mixture was extracted with ethyl acetate (3×25 mL) and the combined organics were dried over magnesium sulfate, filtered and concentrated. The residue was suspended in DCM (15 mL) and L-serine methyl ester hydrochloride (1.762 g, 11.3 mmole) was added. N,N-Diisopropylethylamine (3.00 mL, 17.2 mmole) and EDC·HCl (2.013 g, 10.5 mmole) were added and the mixture was stirred at RT for 18 hours. Water (50 mL) was added and the two layers were separated. The aqueous layer was extracted with DCM (2×15 mL) and the combined organics were dried over magnesium sulfate, filtered and concentrated. The crude residue was purified by silica gel chromatography to provide methyl (2-((S)-3-((tert-butoxycarbonyl)amino)piperidin-1-yl)thiazole-4-carbonyl)-L-serinate as a white solid (1.619 g, 44%). ¹H NMR (400 MHz, CDCl₃): δ 7.99 (d, J=7.5 Hz, 1H), 7.40 (s, 1H), 4.85-4.66 (m, 2H), 4.05 (dt, J=7.2, 3.8 Hz, 2H), 3.82 (s, 4H), 3.74 (dd, J=12.8, 3.5 Hz, 1H), 3.56 (d, J=13.0 Hz, 1H), 3.41 (s, 1H), 3.25 (d, J=9.9 Hz, 1H), 2.96 (s, 1H), 1.92 (ddt, J=11.8, 7.5, 3.4 Hz, 1H), 1.83 (dh, J=10.5, 3.4 Hz, 1H), 1.60 (dtd, J=11.9, 8.0, 3.5 Hz, 1H), 1.45 (s, 9H).

### Methyl O-(tert-butyldimethylsilyl)-N-(2-((S)-3-(tetrahydro-2H-pyran-4-carboxamido)piperidin-1-yl)thiazole-4-carbonyl)-L-serinate (54.3)

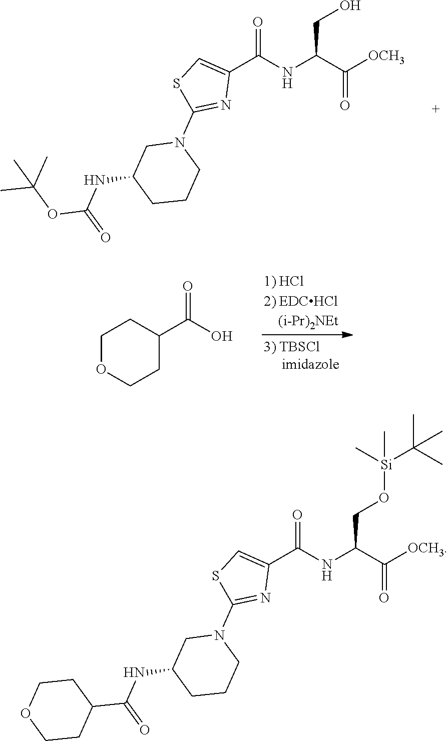 [SMILES: COC(=O)[C@H](CO)NC(=O)c1csc(N2CCC[C@H](NC(=O)OC(C)(C)C)C2)n1 ; O=C(O)C1CCOCC1 ; COC(=O)[C@H](CO[Si](C)(C)C(C)(C)C)NC(=O)c1csc(N2CCC[C@H](NC(=O)C3CCOCC3)C2)n1]

Methyl (2-((,S)-3-((tert-butoxycarbonyl)amino)piperidin-1l-yl)thiazole-4-carbonyl)-L-serinate (0.505 g, 1.18 mmole) was dissolved in DCM (4.6 mL). HCl (4M solution in 1,4-dioxane, 1.20 mL, 4.80 mmole) was added and the mixture was stirred at RT for 4 hours, then was concentrated. The residue was suspended in DCM (2.3 mL) and tetrahydro-2H-pyran-4-carboxylic acid (0.202 g, 1.55 mmole) was added. EDC·HCl (0.279 g, 1.46 mmole) and N,N-diisopropylethyl amine (0.300 mL, 1.72 mmole) were added and the mixture was stirred at RT for 4 days. Water (25 mL) was added and the mixture was extracted with DCM (3×10 mL). The combined organics were dried over magnesium sulfate, filtered and concentrated. The residue was dissolved in DCM (2.3 mL). Imidazole (0094 g, 1.38 mmole) and tert-butyldimethylchlorosilane (0.192 g, 1.27 mmole) were added and the mixture was stirred at RT for 18 hours. Water (25 mL) was added and the mixture was extracted with DCM (3×10 mL). The combined organics were dried over magnesium sulfate, filtered and concentrated. The crude residue was purified by silica gel chromatography to provide methyl O-(tert-butyldimethylsilyl)-N-(2-((S)-3-(tetrahydro-2H-pyran-4-carboxamido)piperidin-1-yl)thiazole-4-carbonyl)-L-serinate as a thick, colorless oil (0.301 g, 46%). DH NMR (400 MHz, CDCl₃): s 7.92 (d, J=8.8 Hz, 1H), 7.40 (s, 1H), 5.71 (d, J=7.7 Hz, 1H), 4.79 (ddd, J=8.8, 3.4, 2.6 Hz, 1H), 4.16 (dd, J=10.0, 2.6 Hz, 2H), 4.00 (ddt, J=11.7, 3.9, 1.5 Hz, 2H), 3.89 (dd, J=10.0, 3.4 Hz, 1H), 3.77 (s, 3H), 3.67-3.5 9 (m, 1H), 3.5 5 (dd, J=12.8, 3.4 Hz, 1H), 3.4 8-3.3 5 (m, 4H), 2.3 2 (tt, J=11.2, 4.3 Hz, 1H), 1.88-1.67 (m, 8H), 0.88 (s, 9H), 0.05 (s, 3H), 0.03 (s, 3H).

### Methyl O-acetyl-N-(-(tert-butyldimethylsilyl)-N-(2-((S)-3-(tetrahydro-2H-pyran-4-carboxamido)piperidin-1-yl)thiazole-4-carbonyl)-L-seryl)-L-serinate (54.4)

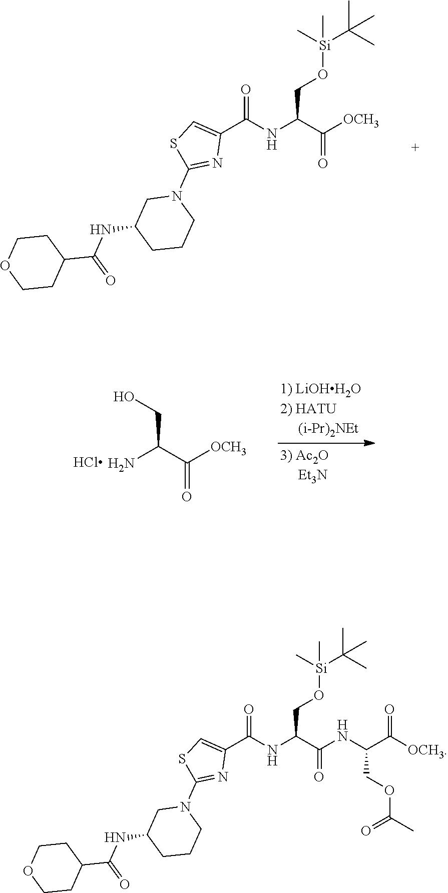 [SMILES: COC(=O)[C@H](CO[Si](C)(C)C(C)(C)C)NC(=O)c1csc(N2CCC[C@H](NC(=O)C3CCOCC3)C2)n1 ; COC(=O)[C@@H](N)CO ; COC(=O)[C@H](COC(C)=O)NC(=O)[C@H](CO[Si](C)(C)C(C)(C)C)NC(=O)c1csc(N2CCC[C@H](NC(=O)C3CCOCC3)C2)n1]

Methyl O-(tert-butyldimethylsilyl)-N-(2-((S′)-3-(tetrahydro-2H-pyran-4-carboxamido)piperidin-1l-yl)thiazole-4-carbonyl)-L-serinate (0.301 g, 0.543 mmole) was dissolved in 4/1/1 THF/methanol/water (2 mL). Lithium hydroxide monohydrate (0.054 g, 1.29 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in DCM (2 mL). L-Serine methyl ester hydrochloride (0.114 g, 0.733 mmole) was added followed by HATU (0.248 g, 0.652 mmole) and N,N-diisopropylethyl amine (0.190 mL, 1.09 mmole). The mixture was stirred at RT for 18 hours and water (25 mL) was added. The mixture was extracted with DCM (3×10 mL) and the combined organics were dried over magnesium sulfate, filtered and concentrated. The residue was dissolved in DCM (2 mL). Triethylamine (0.084 mL, 0.599 mmole) and acetic anhydride (0.057 mL, 0.603 mmole) were added and the solution was stirred at RT for 3 hours. Water (25 mL) was added and the mixture was extracted with DCM (3×10 mL). The combined organics were dried over magnesium sulfate, filtered and concentrated. The crude residue was purified by silica gel chromatography to provide methyl O-acetyl-N-(-(tert-butyldimethylsilyl)-N-(2-((S)-3-(tetrahydro-2H-pyran-4-carboxamido)piperidin-yl)thiazole-4-carbonyl)-L-seryl)-L-serinate as a thick, colorless oil (0.182 g, 49%). 2H NMR (400 MHz, CDCl₃): e 7.92 (d, J=7.2 Hz, 1H), 7.43 (d, J=7.7 Hz, 1H), 7.39 (s, 1H), 5.68 (t, J=7.7 Hz, 1H), 4.84 (dq, J=7.2, 3.6 Hz, 1H), 4.56 (td, J=7.3, 3.8 Hz, 1H), 4.45 (ddd, J=10.6, 6.7, 3.9 Hz, 1H), 4.30 (dd, J=11.4, 3.7 Hz, 1H), 4.19-4.07 (m, 2H), 4.03-3.92 (m, 2H), 3.74 (d, J=3.1 Hz, 3H), 3.73-3.67 (m, 1H), 3.56 (ddd, J=19.7, 11.1, 4.1 Hz, 2H), 3.48-3.31 (m, 4H), 2.29 (tt, J=11.1, 4.5 Hz, 1H), 2.01 (d, J=2.7 Hz, 3H), 1.86-1.65 (m, 8H), 0.90 (s, 9H), 0.12 (s, 3H), 0.10 (s, 3H).

### Methyl (S)-2-(2-(2-(3-(tetrahydro-2H-pyran-4-carboxamido)piperidin-1-yl)thiazole-4-carboxamido)acrylamido)acrylate (54)

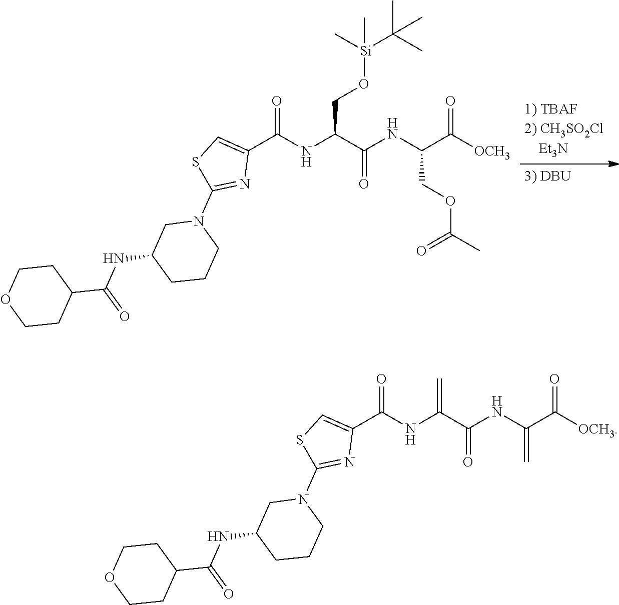 [SMILES: COC(=O)[C@H](COC(C)=O)NC(=O)[C@H](CO[Si](C)(C)C(C)(C)C)NC(=O)c1csc(N2CCC[C@H](NC(=O)C3CCOCC3)C2)n1 ; C=C(NC(=O)c1csc(N2CCC[C@H](NC(=O)C3CCOCC3)C2)n1)C(=O)NC(=C)C(=O)OC]

Methyl O-acetyl-N-(0-(tert-butyldimethylsilyl)-N-(2-((S′)-3-(tetrahydro-2H-pyran-4-carboxamido)piperidin-1l-yl)thiazole-4-carbonyl)-L-seryl)-L-serinate (0.182 g, 0.266 mmole) was dissolved in THF (2 mL). TBAF (1M solution in THF, 0.670 mL, 0.670 mmole) was added and the solution was stirred at RT for 18 hours. Water (25 mL) was added and the mixture was extracted with ethyl acetate (3×10 mL). The combined organics were dried over magnesium sulfate, filtered and concentrated. The residue was dissolved in DCM (2 mL) and cooled to 0° C. Triethylamine (0.056 mL, 0.400 mmole) and methanesulfonyl chloride (0.031 mL, 0.401 mmole) were added and the mixture was stirred at 0° for 90 minutes. Water (25 mL) was added and the mixture was extracted with DCM (3×10 mL). The combined organics were dried over magnesium sulfate, filtered and concentrated. The residue was dissolved in THF (2 mL) and cooled to 0° C. DBU (0.060 mL, 0.401 mmole) was added and the mixture was stirred at 0° for 90 minutes. Water (25 mL) was added and the precipitated solid was collected by filtration to provide methyl (S)-2-(2-(2-(3-(tetrahydro-2H-pyran-4-carboxamido)piperidin-1l-yl)thiazole-4-carboxamido)acrylamido)acrylate as a white solid (0.045 g, 34%). ¹H NMR (400 MHz, CDCl₃): δ 9.69 (s, 1H), 8.51 (s, 1H), 7.45 (s, 1H), 6.72 (d, J=2.1 Hz, 1H), 6.65 (s, 1H), 6.00 (d, J=1.2 Hz, 1H), 5.78 (d, J=7.5 Hz, 1H), 5.44 (t, J=1.9 Hz, 1H), 4.17 (s, 1H), 4.04-3.94 (m, 2H), 3.89 (s, 3H), 3.67-3.53 (m, 3H), 3.53-3.43 (m, 1H), 3.38 (tt, J=11.4, 3.3 Hz, 2H), 2.32 (tt, J =11.1, 4.4 Hz, 1H), 1.91-1.66 (m, 8H).

### Compound 55: Methyl 2-(2-(2-(4-(2-methoxyacetamido)piperidin-1l-yl)thiazole-4-carboxamido)acrylamido)acrylate

### Methyl O-(tert-butyldimethylysilyl)-N-(2-(4-(2-methoxyacetamido)piperidin-4-yl)thiazole-4-carbonyl)-L-serinate (55.1)

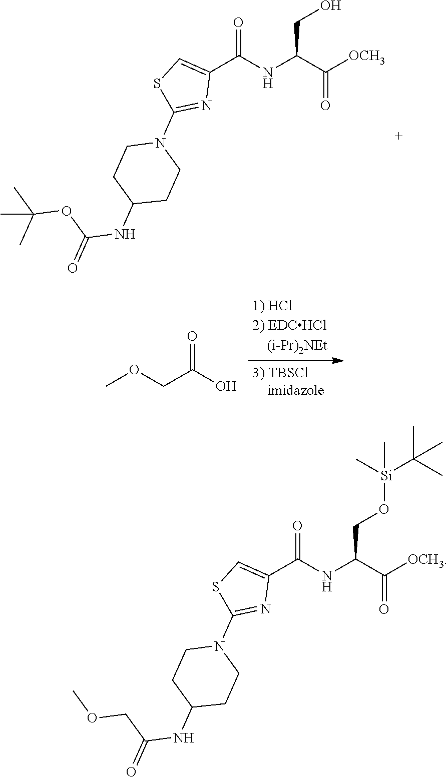

Methyl (2-(4-((tert-butoxycarbonyl)amino)piperidin-1-yl)thiazole-4-carbonyl)-L-serinate (Example 46.2, 0.449 g, 1.05 mmole) was dissolved in DCM (4.2 mL). HCl (4M solution in 1,4-dioxane, 1.05 mL, 4.20 mmole) was added and the mixture was stirred at RT for 4 hours, then was concentrated. The residue was suspended in DCM (2.1 mL) and 2-methoxyacetic acid (0.105 mL, 1.37 mmole) was added. EDC·HCl (0.245 g, 1.28 mmole) and N,N-diisopropylethylamine (0.370 mL, 2.12 mmole) were added and the mixture was stirred at RT for 18 hours. Water (25 mL) was added and the mixture was extracted with DCM (3×10 mL). The combined organics were dried over magnesium sulfate, filtered and concentrated. The residue was dissolved in DCM (2.1 mL) and imidazole (0.082 g, 1.20 mmole) and tert-butyldimethylchlorosilane (0.175 g, 1.16 mmole) were added. The mixture was stirred at RT for 18 hours and water (25 mL) was added. The mixture was extracted with DCM (3×10 mL) and the combined organics were dried over magnesium sulfate, filtered and concentrated. The crude residue was purified by silica gel chromatography to provide methyl O-(tert-butyldimethylsilyl)-N-(2-(4-(2-methoxyacetamido)piperidin-1-yl)thiazole-4-carbonyl)-L-serinate as a white solid (0.325 g, 60%). ¹H NMR (400 MHz, CDCl₃): δ 7.95 (d, J=8.8 Hz, 1H), 7.39 (s, 1H), 6.47 (d, J=8.2 Hz, 1H), 4.78 (ddd, J=8.9, 3.3, 2.6 Hz, 1H), 4.20-4.14 (m, 1H), 4.10-4.05 (m, 1H), 4.05-3.92 (m, 2H), 3.91-3.86 (m, 1H), 3.89 (s, 2H), 3.76 (s, 3H), 3.43 (s, 3H), 3.17 (dddd, J=13.2, 11.3, 8.1, 2.9 Hz, 2H), 2.07-1.99 (m, 2H), 1.57 (tddd, J=11.5, 10.2, 5.8, 4.4 Hz, 2H), 0.88 (s, 9H), 0.05 (s, 3H), 0.03 (s, 3H).

### Methyl O-acetyl-N—(O-(tert-butyldimethylsilyl)-N-(2-(4-(2-methoxyacetamido)piperidin-1-yl)thiazole-4-carbonyl)-L-seryl)-L-serinate (55.2)

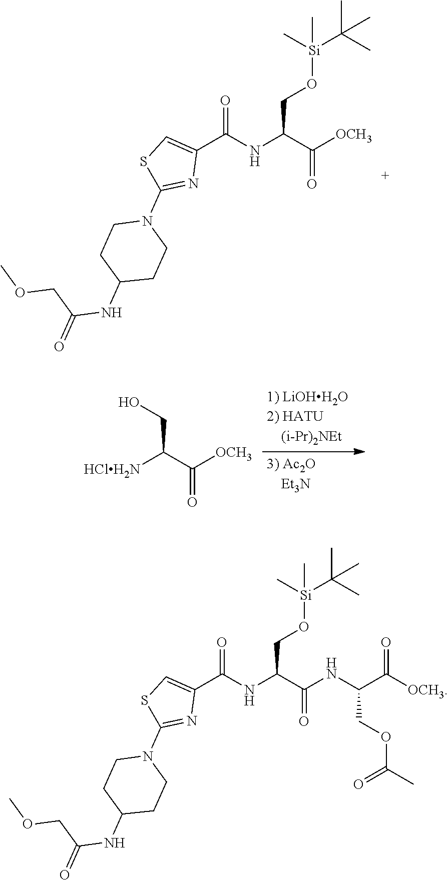

Methyl O-(tert-butyldimethylsilyl)-N-(2-(4-(2-methoxyacetamido)piperidin-1-yl)thiazole-4-carbonyl)-L-serinate (0.325 g, 0.631 mmole) was dissolved in 4/1/1 THF/methanol/water (2 mL). Lithium hydroxide monohydrate (0.057 g, 1.36 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in DCM (2 mL). L-Serine methyl ester hydrochloride (0.133 g, 0.855 mmole) was added followed by HATU (0.291 g, 0.765 mmole) and N,N-diisopropylethylamine (0.220 mL, 1.26 mmole). The mixture was stirred at RT for 18 hours and water (25 mL) was added. The mixture was extracted with DCM (3×10 mL) and the combined organics were dried over magnesium sulfate, filtered and concentrated. The residue was dissolved in DCM (2 mL). Triethylamine (0.098 mL, 0.699 mmole) and acetic anhydride (0.066 mL, 0.698 mmole) were added and the solution was stirred at RT for 18 hours. Water (25 mL) was added and the mixture was extracted with DCM (3×10 mL). The combined organics were dried over magnesium sulfate, filtered and concentrated. The crude residue was purified by silica gel chromatography to provide methyl 0-acetyl-N-(0-(tert-butyldimethylsilyl)-N-(2-(4-(2-methoxyacetamido)piperidin-1-yl)thiazole-4-carbonyl)-L-seryl)-L-serinate as a thick, pale yellow gel (0.254 g, 63%). ¹H NMR (400 MHz, CDCl₃): δ 7.96 (d, J=7.1 Hz, 1H), 7.41 (d, J=7.7 Hz, 1H), 7.38 (d, J=2.8 Hz, 1H), 6.44 (d, J=8.2 Hz, 1H), 4.85 (dt, J=7.7, 3.8 Hz, 1H), 4.56 (td, J=7.2, 3.6 Hz, 1H), 4.50-4.40 (m, 1H), 4.29 (dd, J=11.4, 3.7 Hz, 1H), 4.20-4.13 (m, 1H), 4.13-4.01 (m, 1H), 3.96 (t, J=14.4 Hz, 2H), 3.87 (s, 2H), 3.74 (d, J=2.1 Hz, 3H), 3.72-3.65 (m, 1H), 3.40 (s, 3H), 3.14 (tdd, J=13.0, 6.1, 3.0 Hz, 2H), 2.06-1.97 (m, 2H), 2.00 (s, 3H), 1.57-1.47 (m, 2H), 0.90 (s, 9H), 0.11 (s, 3H), 0.10 (s, 3H).

### Methyl 2-(2-(2-(4-(2-methoxyacetamido)piperidin-1-yl)thiazole-4-carboxamido)acrylamido)acrylate (55)

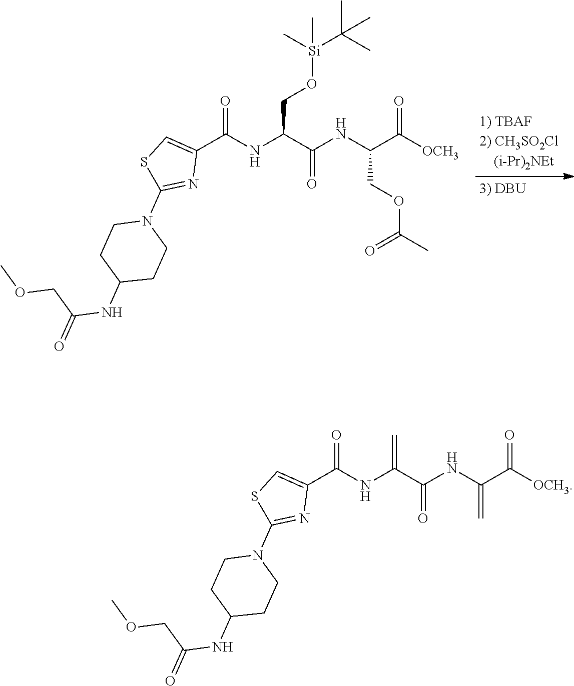 [SMILES: COCC(=O)NC1CCN(c2nc(C(=O)N[C@@H](CO[Si](C)(C)C(C)(C)C)C(=O)N[C@@H](COC(C)=O)C(=O)OC)cs2)CC1 ; C=C(NC(=O)c1csc(N2CCC(NC(=O)COC)CC2)n1)C(=O)NC(=C)C(=O)OC]

Methyl O-acetyl-N-(O-(tert-butyldimethylsilyl)-N-(2-(4 (2-methoxyacetamido)piperidin-yl)thiazole-4-carbonyl)-L-seryl)-L-serinate (0.254 g, 0.395 mmole) was dissolved in THE (2 mL). TBAF (1M solution in THF, 1.00 mL, 1.00 mmole) was added and the solution was stirred at RT for 18 hours. Water (25 mL) was added and the mixture was extracted with ethyl acetate (3×10 mL). The combined organics were dried over magnesium sulfate, filtered and concentrated. The residue was dissolved in DCM (2 mL) and cooled to 0° C. Triethylamine (0.083 mL, 0.592 mmole) and methanesulfonyl chloride (0.046 mL, 0.594 mmole) were added and the mixture was stirred at 00 for 90 minutes. Water (25 mL) was added and the mixture was extracted with DCM (3×10 mL). The combined organics were dried over magnesium sulfate, filtered and concentrated. The residue was dissolved in THE (2 mL) and cooled to 0° C. DBU (0.090 mL, 0.602 mmole) was added and the mixture was stirred at 0° for 90 minutes. Water (25 mL) was added and the mixture was extracted with ethyl acetate (3×10 mL). The combined organics were dried over magnesium sulfate, filtered and concentrated. The crude residue was purified by silica gel chromatography to provide methyl 2-(2-(2-(4-(2-methoxyacetamido)piperidin-1-yl)thiazole-4-carboxamido)acrylamido)acrylate as a white solid (0.093 g, 52%). ¹H NMR (400 MHz, CDCl₃): δ 9.72 (s, 1H), 8.51 (s, 1H), 7.43 (s, 1H), 6.72 (d, J=2.1 Hz, 1H), 6.66 (s, 1H), 6.47 (d, J=8.3 Hz, 1H), 6.01 (d, J=1.3 Hz, 1H), 5.44 (t, J=1.8 Hz, 1H), 4.10 (ddd, J=14.2, 7.3, 3.5 Hz, 1H), 4.01 (dt, J=13.4, 3.9 Hz, 2H), 3.90 (s, 2H), 3.89 (s, 3H), 3.42 (s, 3H), 3.20 (ddd, J=13.3, 11.7, 3.0 Hz, 2H), 2.12-2.00 (m, 2H), 1.66-1.52 (m, 2H).

### Compound 56: Tert-butyl (cis)-4-(4-((3-((3-methoxy-3-oxoprop-1-en-2-yl)amino)-3-oxoprop-1-en-2-yl)carbamoyl)thiazol-2-yl)-2,6-dimethylpiperazine-1-carboxylate

### Ethyl 2-((cis)-4-(tert-butoxycarbonyl)-3,5-dimethylpiperazin-1-yl)thiazole-4-carboxylate (56.1)

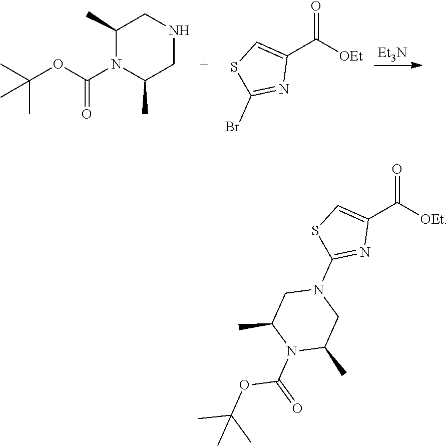 [SMILES: "Et3N" ; C[C@@H]1CNC[C@H](C)N1C(=O)OC(C)(C)C ; CCOC(=O)c1csc(Br)n1 ; CCOC(=O)c1csc(N2C[C@@H](C)N(C(=O)OC(C)(C)C)[C@@H](C)C2)n1]

Tert-butyl (cis)-2,6-dimethylpiperazine-1-carboxylate (1.033 g, 4.82 mmole) was dissolved in DMA (5 mL). Ethyl 2-bromothiazole-4-carboxylate (1.109 g, 4.70 mmole) and triethylamine (0.720 mL, 5.14 mmole) were added and the mixture was heated to 80° C. for 24 hours, then was cooled to RT and diluted with ethyl acetate. The organics were washed with water (3×15 mL) and dried over magnesium sulfate, then filtered and concentrated. The crude residue was purified by silica gel chromatography to provide ethyl 2-((cis)-4-(tert-butoxycarbonyl)-3,5-dimethylpiperazin-1-yl)thiazole-4-carboxylate as a pale yellow solid (0.699 g, 39%). ¹H NMR (400 MHz, CDCl₃): δ 7.45 (s, 1H), 4.35 (q, J=7.1 Hz, 2H), 4.29 (tdt, J=6.9, 4.7, 1.9 Hz, 2H), 3.86-3.76 (m, 2H), 3.26 (dd, J=12.7, 4.7 Hz, 2H), 1.49 (s, 9H), 1.37 (t, J=7.1 Hz, 3H), 1.31 (d, J=6.8 Hz, 6H)

### Tert-butyl (cis)-4-(4-(((S)-3-((tert-butyldimethylsilyl)oxy)-1-methoxy-1-oxopropan-2-yl)carbamoyl)thiazol-2-yl)-2,6-dimethylpiperazine-1-carboxylate (56.2)

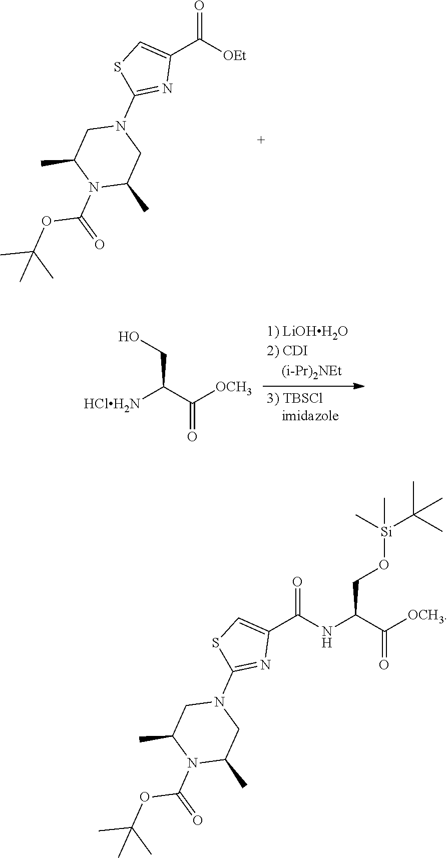

Ethyl 2-((cis)-4-(tert-butoxycarbonyl)-3,5-dimethylpiperazin-1-yl)thiazole-4-carboxylate (0.699 g, 1.89 mmole) was dissolved in 4/1/1 THF/methanol/water (3 mL). Lithium hydroxide monohydrate (0.167 g, 3.98 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in THE (4 mL) and CDI (0.330 g, 2.04 mmole) was added portionwise (gas evolution was observed). The solution was stirred at RT for 60 minutes and L-serine methyl ester hydrochloride (0.382 g, 2.46 mmole) was added, followed by N,N-diisopropylethylamine (0.660 mL, 3.79 mmole). The mixture was stirred at RT for 3 days and water (25 mL) was added. The mixture was extracted with ethyl acetate (3×10 mL) and the combined organics were dried over magnesium sulfate, filtered and concentrated. The residue was dissolved in DCM (4 mL). Imidazole (0.150 g, 2.20 mmole) and tert-butyldimethylchlorosilane (0.336 g, 2.23 mmole) were added and the mixture was stirred at RT for 3 hours. Water (25 mL) was added and the mixture was extracted with DCM (3×10 mL). The combined organics were dried over magnesium sulfate, filtered and concentrated. The crude residue was purified by silica gel chromatography to provide tert-butyl (cis)-4-(4-(((S)-3-((tert-butyldimethylsilyl)oxy)-1-methoxy-1-oxopropan-2-yl)carbamoyl)thiazol-2-yl)-2,6-dimethylpiperazine-1-carboxylate as a colorless oil (0.849 g, 81%). ¹H NMR (400 MHz, CDCl₃): δ 7.93 (d, J=8.7 Hz, 1H), 7.39 (s, 1H), 4.79 (dt, J=8.8, 3.0 Hz, 1H), 4.29 (qdd, J=6.8, 4.8, 1.5 Hz, 2H), 4.20-4.14 (m, 1H), 3.89 (dd, J=10.1, 3.3 Hz, 1H), 3.85-3.71 (m, 2H), 3.77 (s, 3H), 3.24 (dd, J=12.7, 4.6 Hz, 2H), 1.50 (s, 9H), 1.35-1.27 (m, 6H), 0.88 (s, 9H), 0.06 (s, 3H), 0.03 (s, 3H)

### Tert-butyl 4-(4-(((cis)-9-(methoxycarbonyl)-2,2,3,3-tetramethyl-7,12-dioxo-4,11-dioxa-8-aza-3-silatridecan-6-yl)carbamoyl)thiazol-2-yl)-2,6-dimethylpiperazine-1-carboxylate (56.3)

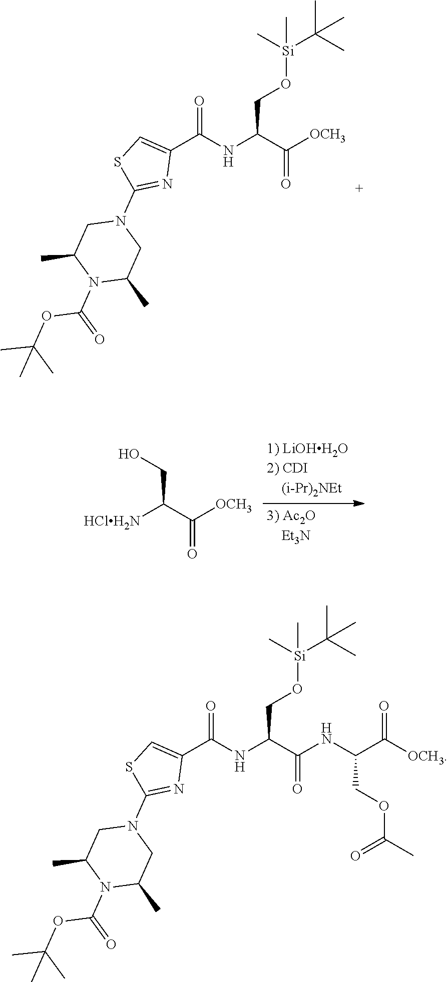

Tert-butyl (cis)-4-(4-(((S)-3-((tert-butyldimethylsilyl)oxy)-1-methoxy-1-oxopropan-2-yl)carbamoyl)thiazol-2-yl)-2,6-dimethylpiperazine-1-carboxylate (0.849 g, 1.52 mmole) was dissolved in 4/1/1 THF/methanol/water (3 mL). Lithium hydroxide monohydrate (0.126 g, 3.00 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in THE (3 mL) and CDI (0.263 g, 1.62 mmole) was added portionwise (gas evolution was observed). The solution was stirred at RT for 60 minutes before L-serine methyl ester hydrochloride (0.306 g, 1.97 mmole) and N,N-diisopropylethylamine (0.530 mL, 3.04 mmole) were added. The mixture was stirred at RT for 18 hours and water (25 mL) was added. The mixture was extracted with DCM (3×10 mL). The combined organics were dried over magnesium sulfate, filtered and concentrated. The residue was dissolved in DCM (3 mL) and triethylamine (0.235 mL, 1.68 mmole) and acetic anhydride (0.160 mL, 1.69 mmole) were added. The mixture was stirred at RT for 18 hours and water (25 mL) was added. The mixture was extracted with DCM (3×10 mL) and the combined organics were dried over magnesium sulfate, filtered and concentrated. The crude residue was purified by silica gel chromatography to provide tert-butyl 4-(4-(((cis)-9-(methoxycarbonyl)-2,2,3,3-tetramethyl-7,12-dioxo-4,11-dioxa-8-aza-3-silatridecan-6-yl)carbamoyl)thiazol-2-yl)-2,6-dimethylpiperazine-1-carboxylate as a thick, colorless oil (0.593 g, 57%). ¹H NMR (400 MHz, CDCl₃): δ 7.96 (d, J=7.2 Hz, 1H), 7.43 (d, J=7.7 Hz, 1H), 7.41 (s, 1H), 4.87 (tt, J=6.6, 3.8 Hz, 1H), 4.63-4.53 (m, 1H), 4.52-4.45 (m, 1H), 4.30 (ddt, J=12.5, 7.1, 4.6 Hz, 3H), 4.19 (ddd, J=9.9, 3.7, 1.7 Hz, 1H), 3.80 (dd, J=4.7, 2.1 Hz, 2H), 3.76 (s, 3H), 3.75-3.69 (m, 1H), 3.24 (ddd, J=13.0, 4.7, 2.0 Hz, 2H), 2.02 (s, 3H), 1.49 (s, 9H), 1.31 (dt, J=6.9, 2.0 Hz, 6H), 0.93 (s, 9H), 0.14 (s, 3H), 0.12 (s, 3H)

### Tert-butyl (cis)-4-(4-((3-((3-methoxy-3-oxoprop-1-en-2-yl)amino)-3-oxoprop-1-en-2-yl)carbamoyl)thiazol-2-yl)-2,6-dimethylpiperazine-1-carboxylate (56)

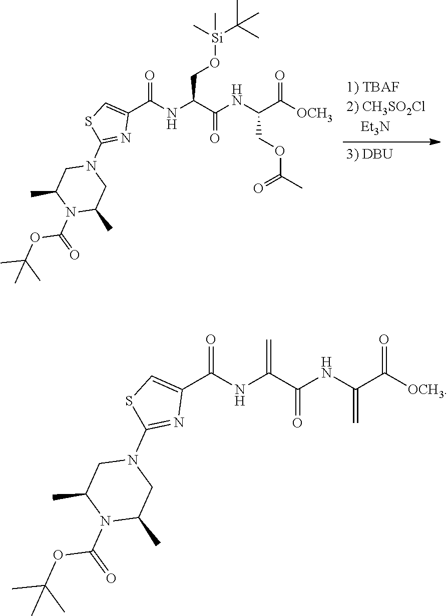 [SMILES: COC(=O)[C@H](COC(C)=O)NC(=O)[C@H](CO[Si](C)(C)C(C)(C)C)NC(=O)c1csc(N2C[C@@H](C)N(C(=O)OC(C)(C)C)[C@@H](C)C2)n1 ; C=C(NC(=O)c1csc(N2C[C@@H](C)N(C(=O)OC(C)(C)C)[C@@H](C)C2)n1)C(=O)NC(=C)C(=O)OC]

Tert-butyl 4-(4-(((cis)-9-(methoxycarbonyl)-2,2,3,3-tetramethyl-7,12-dioxo-4,11-dioxa-8-aza-3-silatridecan-6-yl)carbamoyl)thiazol-2-yl)-2,6-dimethylpiperazine-1-carboxylate (0.593 g, 0.865 mmole) was dissolved in THE (1.8 mL). TBAF (1M solution in THF, 2.20 mL, 2.20 mmole) was added and the solution was stirred at RT for 18 hours. Water (25 mL) was added and the mixture was extracted with ethyl acetate (3×10 mL). The combined organics were dried over magnesium sulfate, filtered and concentrated. The residue was dissolved in DCM (1.8 mL) and cooled to 0° C. Triethylamine (0.185 mL, 1.32 mmole) was added followed by methanesulfonyl chloride (0.100 mL, 1.29 mmole) and the mixture was stirred at 0° for 60 minutes. Water (25 mL) was added and the mixture was extracted with DCM (3×10 mL). The combined organics were dried over magnesium sulfate, filtered and concentrated. The residue was dissolved in THE (1.8 mL) and cooled to 0° C. DBU (0.200 mL, 1.34 mmole) was added and the mixture was stirred at 0° for 60 minutes. Water (25 mL) was added and the mixture was extracted with ethyl acetate (3×10 mL). The combined organics were dried over magnesium sulfate, filtered and concentrated. The crude residue was purified by silica gel chromatography to provide tert-butyl (cis)-4-(4-((3-((3-methoxy-3-oxoprop-1-en-2-yl)amino)-3-oxoprop-1-en-2-yl)carbamoyl)thiazol-2-yl)-2,6-dimethylpiperazine-1-carboxylate as a white solid (0.172 g, 40%). ¹H NMR (400 MHz, CDCl₃): δ 9.72 (s, 1H), 8.51 (s, 1H), 7.43 (s, 1H), 6.72 (d, J=2.2 Hz, 1H), 6.67 (s, 1H), 6.01 (d, J=1.3 Hz, 1H), 5.44 (t, J=1.9 Hz, 1H), 4.38-4.25 (m, 2H), 3.89 (s, 3H), 3.86-3.79 (m, 2H), 3.27 (dd, J=12.9, 4.6 Hz, 2H), 1.46 (s, 9H), 1.32 (d, J=6.9 Hz, 6H).

### Compound 57: Tert-butyl (S)-4-(4-((3-((3-methoxy-3-oxoprop-1-en-2-yl)amino)-3-oxoprop-1-en-2-yl)carbamoyl)thiazol-2-yl)-2-methylpiperazine-1-carboxylate

### Ethyl (S)-2-(4-(tert-butoxycarbonyl)-3-methylpiperazin-1-yl)thiazole-4-carboxylate (57.1)

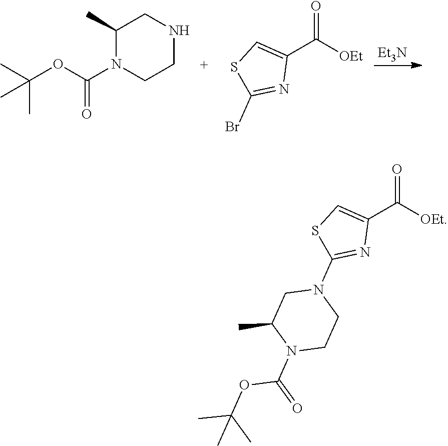 [SMILES: "Et3N" ; C[C@H]1CNCCN1C(=O)OC(C)(C)C ; CCOC(=O)c1csc(Br)n1 ; CCOC(=O)c1csc(N2CCN(C(=O)OC(C)(C)C)[C@@H](C)C2)n1]

Tert-butyl (S)-2-methylpiperazine-1-carboxylate (1.501 g, 7.49 mmole) was dissolved in DMA (7.5 mL). Ethyl 2-bromothiazole-4-carboxylate (1.864 g, 7.90 mmole) and triethylamine (1.30 mL, 9.28 mmole) were added and the mixture was stirred at 80° C. for 20 hours. The mixture was cooled to RT and diluted with ethyl acetate (25 mL). The mixture was washed with water (3×20 mL) and the organics were dried over magnesium sulfate, filtered and concentrated. The crude residue was purified by silica gel chromatography to provide ethyl (S)-2-(4-(tert-butoxycarbonyl)-3-methylpiperazin-1-yl)thiazole-4-carboxylate as an orange oil (1.842 g, 69%). ¹H NMR (400 MHz, CDCl₃): δ 7.45 (s, 1H), 4.42-4.34 (m, 1H), 4.35 (q, J=7.1 Hz, 2H), 4.04-3.91 (m, 2H), 3.68 (dt, J=12.8, 1.8 Hz, 1H), 3.38-3.30 (m, 1H), 3.24 (ddd, J=13.3, 11.9, 3.5 Hz, 1H), 3.09 (td, J=12.1, 3.7 Hz, 1H), 1.48 (s, 9H), 1.37 (t, J=7.1 Hz, 3H), 1.25-1.19 (m, 3H)

### Tert-butyl (S)-4-(4-(((S)-3-((tert-butyldimethylsilyl)oxy)-1-methoxy-1-oxopropan-2-yl)carbamoyl)thiazol-2-yl)-2-methylpiperazine-1-carboxylate (57.2)

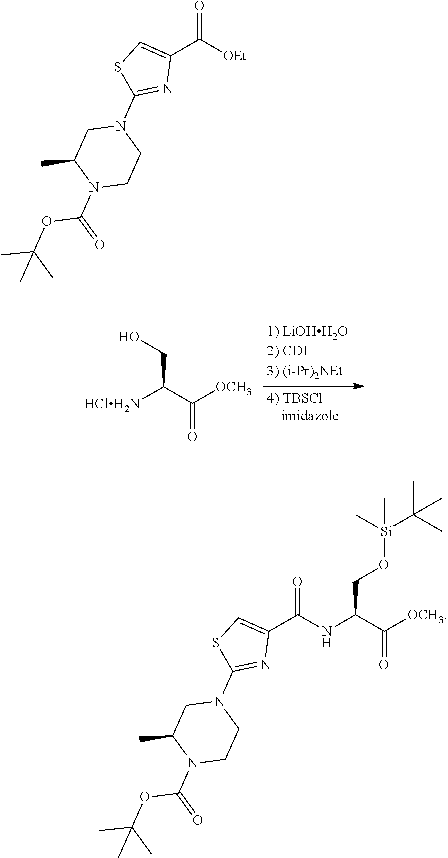

Ethyl (S)-2-(4-(tert-butoxycarbonyl)-3-methylpiperazin-1-yl)thiazole-4-carboxylate (0.605 g, 1.70 mmole) was dissolved in 4/1/1 THF/methanol/water (3.4 mL). Lithium hydroxide monohydrate (0.143 g, 3.41 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in THF (3.4 mL) and CDI (0.291 g, 1.79 mmole) was added portionwise (gas evolution was observed). The solution was stirred at RT for 60 minutes before L-serine methyl ester hydrochloride (0.343 g, 2.20 mmole) and N,N-diisopropylethyl amine (0.350 mL, 2.01 mmole) were added. The mixture was stirred at RT for 18 hours and water (25 mL) was added. The mixture was extracted with ethyl acetate (3×10 mL) and the combined organics were dried over magnesium sulfate, filtered and concentrated. The residue was dissolved in DCM (3.4 mL) and imidazole (0.126 g, 1.85 mmole) and tert-butyldimethylchlorosilane (0.285 g, 1.89 mmole) were added. The mixture was stirred at RT for 90 minutes and water (25 mL) was added. The mixture was extracted with DCM (3×10 mL) and the combined organics were dried over magnesium sulfate, filtered and concentrated. The crude residue was purified by silica gel chromatography to provide tert-butyl (S)-4-(4-(((S)-3-((tert-butyldimethylsilyl)oxy)-1-methoxy-1-oxopropan-2-yl)carbamoyl)thiazol-2-yl)-2-methylpiperazine-1-carboxylate as a thick, pale yellow gel (0.636 g, 69%). ¹H NMR (400 MHz, CDCl₃): δ 7.94 (d, J=8.7 Hz, 1H), 7.39 (s, 1H), 4.78 (ddd, J=8.8, 3.4, 2.6 Hz, 1H), 4.38 (s, 1H), 4.16 (dd, J=10.0, 2.6 Hz, 1H), 4.00-3.92 (m, 1H), 3.89 (dd, J=10.0, 3.4 Hz, 2H), 3.76 (s, 3H), 3.68 (dt, J=12.7, 1.9 Hz, 1H), 3.34-3.26 (m, 1H), 3.26-3.16 (m, 1H), 3.09 (td, J=12.1, 3.6 Hz, 1H), 1.49 (s, 9H), 1.25 (dd, J=7.0, 4.4 Hz, 3H), 0.89 (s, 9H), 0.05 (s, 3H), 0.03 (s, 3H)

### Tert-butyl (S)-4-(4-(((6S,9S)-9-(methoxycarbonyl)-2,2,3,3-tetramethyl-7,12-dioxo-4,11-dioxa-8-aza-3-silatridecan-6-yl)carbamoyl)thiazol-2-yl)-2-methylpiperazine-1-carboxylate (57.3)

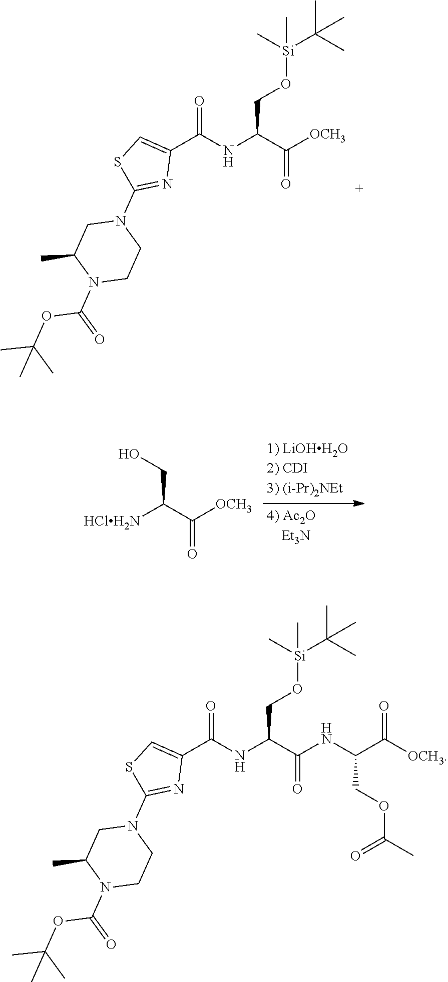

Tert-butyl (S)-4-(4-(((S)-3-((tert-butyldimethylsilyl)oxy)-1-methoxy-1-oxopropan-2-yl)carbamoyl)thiazol-2-yl)-2-methylpiperazine-1-carboxylate (0.636 g, 1.17 mmole) was dissolved in 4/1/1 THF/methanol/water (2.4 mL). Lithium hydroxide monohydrate (0.102 g, 2.43 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was suspended in THE (2.4 mL). CDI (0.202 g, 1.25 mmole) was added portionwise (gas evolution was observed) and the solution was stirred at RT for 45 minutes. L-Serine methyl ester hydrochloride (0.239 g, 1.54 mmole) was added followed by N,N-diisopropylethylamine (0.270 mL, 1.55 mmole) and the mixture was stirred at RT for 18 hours. Water (25 mL) was added and the mixture was extracted with ethyl acetate (3×10 mL). The combined organics were dried over magnesium sulfate, filtered and concentrated. The residue was dissolved in DCM (2.4 mL), then triethylamine (0.180 mL, 1.28 mmole) and acetic anhydride (0.120 mL, 1.27 mmole) were added. The mixture was stirred at RT for 3 hours and water (25 mL) was added. The mixture was extracted with DCM (3×10 mL) and the combined organics were dried over magnesium sulfate, filtered and concentrated. The crude residue was purified by silica gel chromatography to provide tert-butyl (S)-4-(4-(((6S,9S)-9-(methoxycarbonyl)-2,2,3,3-tetramethyl-7,12-dioxo-4,11-dioxa-8-aza-3-silatridecan-6-yl)carbamoyl)thiazol-2-yl)-2-methylpiperazine-1-carboxylate as a pale yellow oil (0.471 g, 60%). ¹H NMR (400 MHz, CDCl₃): δ 7.98 (d, J=7.0 Hz, 1H), 7.44 (d, J=7.8 Hz, 1H), 7.41 (d, J=2.8 Hz, 1H), 4.88 (dt, J=7.6, 3.7 Hz, 1H), 4.58 (td, J=7.2, 3.6 Hz, 1H), 4.47 (ddd, J=11.4, 10.1, 4.0 Hz, 1H), 4.42-4.35 (m, 1H), 4.31 (dd, J=11.4, 3.6 Hz, 1H), 4.19 (ddd, J=9.8, 3.6, 2.1 Hz, 1H), 3.99-3.87 (m, 2H), 3.83-3.70 (m, 1H), 3.77 (s, 3H), 3.68 (dd, J=12.8, 2.0 Hz, 1H), 3.30 (dd, J=12.9, 4.1 Hz, 1H), 3.28-3.17 (m, 1H), 3.13-3.04 (m, 1H), 2.02 (s, 3H), 1.49 (s, 9H), 1.25-1.21 (m, 3H), 0.93 (s, 9H), 0.14 (s, 3H), 0.12 (s, 3H)

### Tert-butyl (S)-4-(4-((3-((3-methoxy-3-oxoprop-1-en-2-yl)amino)-3-oxoprop-1-en-2-yl)carbamoyl)thiazol-2-yl)-2-methylpiperazine-1-carboxylate (57)

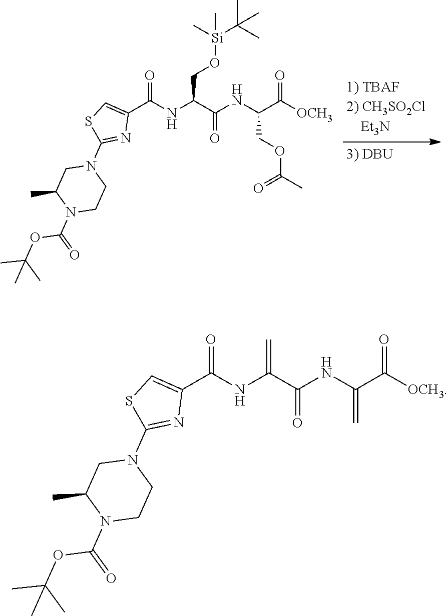 [SMILES: COC(=O)[C@H](COC(C)=O)NC(=O)[C@H](CO[Si](C)(C)C(C)(C)C)NC(=O)c1csc(N2CCN(C(=O)OC(C)(C)C)[C@@H](C)C2)n1 ; C=C(NC(=O)c1csc(N2CCN(C(=O)OC(C)(C)C)[C@@H](C)C2)n1)C(=O)NC(=C)C(=O)OC]

Tert-butyl (S)-4-(4-(((6S,9S)-9-(methoxycarbonyl)-2,2,3,3-tetramethyl-7,12-dioxo-4,11-dioxa-8-aza-3-silatridecan-6-yl)carbamoyl)thiazol-2-yl)-2-methylpiperazine-1-carboxylate (0.471 g, 0.701 mmole) was dissolved in THE (2.8 mL). TBAF (1M solution in THF, 1.80 mL, 1.80 mmole) was added and the solution was stirred at RT for 18 hours. Water (25 mL) was added and the mixture was extracted with ethyl acetate (3×10 mL). The combined organics were dried over magnesium sulfate, filtered and concentrated. The residue was dissolved in DCM (2.8 mL) and cooled to 0° C. Triethylamine (0.150 mL, 1.07 mmole) was added followed by methanesulfonyl chloride (0.082 mL, 1.06 mmole) and the mixture was stirred at 0° for 60 minutes. Water (25 mL) was added and the mixture was extracted with DCM (3×10 mL). The combined organics were dried over magnesium sulfate, filtered and concentrated. The residue was dissolved in THE (2.8 mL) and cooled to 0° C. DBU (0.160 mL, 1.07 mmole) was added and the mixture was stirred at 0° for 60 minutes. Water (25 mL) was added and the mixture was extracted with ethyl acetate (3×10 mL). The combined organics were dried over magnesium sulfate, filtered and concentrated. The crude residue was purified by silica gel chromatography to provide tert-butyl (S)-4-(4-((3-((3-methoxy-3-oxoprop-1-en-2-yl)amino)-3-oxoprop-1-en-2-yl)carbamoyl)thiazol-2-yl)-2-methylpiperazine-1-carboxylate as a white solid (0.115 g, 34%). ¹H NMR (400 MHz, CDCl₃): δ 9.72 (s, 1H), 8.51 (s, 1H), 7.44 (s, 1H), 6.72 (d, J=2.1 Hz, 1H), 6.67 (s, 1H), 6.01 (d, J=1.3 Hz, 1H), 5.44 (t, J=1.9 Hz, 1H), 4.41 (s, 1H), 4.04-3.93 (m, 2H), 3.89 (s, 3H), 3.70 (dt, J=12.8, 1.8 Hz, 1H), 3.32 (dd, J=12.7, 4.1 Hz, 1H), 3.24 (ddd, J=13.5, 11.9, 3.6 Hz, 1H), 3.11 (td, J=12.2, 3.8 Hz, 1H), 1.49 (s, 9H), 1.25 (d, J=7.0, 3H).

### Compound 58: Methyl 2-(2-(2-(4-(1,4-dioxane-2-carboxamido)piperidin-1-yl)thiazole-4-carboxamido)acrylamido)acrylate

### Methyl N-(2-(4-(1,4-dioxane-2-carboxamido)piperidin-1-yl)thiazole-4-carbonyl)-O-(tert-butyldimethylsilyl)-L-serinate (58.1)

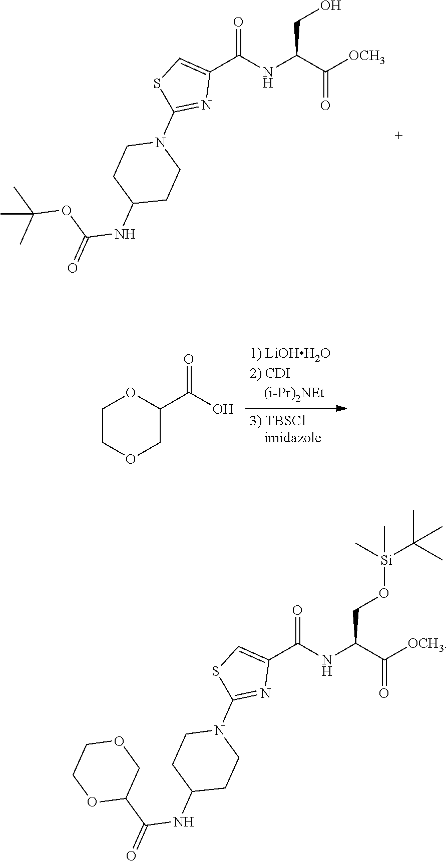 [SMILES: COC(=O)[C@H](CO)NC(=O)c1csc(N2CCC(NC(=O)OC(C)(C)C)CC2)n1 ; O=C(O)C1COCCO1 ; COC(=O)[C@H](CO[Si](C)(C)C(C)(C)C)NC(=O)c1csc(N2CCC(NC(=O)C3COCCO3)CC2)n1]

Methyl (2-(4-((tert-butoxycarbonyl)amino)piperidin-1-yl)thiazole-4-carbonyl)-L-serinate (Example 46.2, 0.507 g, 1.18 mmole) was dissolved in DCM (5 ML). HCl (4M solution in 1,4-dioxane, 1.20 mL, 4.80 mmole) was added and the mixture was stirred at RT for 2 hours, then was concentrated. The residue was suspended in DCM (2.4 mL) and 1,4-dioxane-2-carboxylic acid (0.199 g, 1.51 mmole) was added. EDC·HCl (0.289 g, 1.51 mmole) and N,N-diisopropylethylamine (0.270 mL, 1.55 mmole) were added and the mixture was stirred at RT for 3 days. Water (25 mL) was added and the mixture was extracted with DCM (3×10 mL). The combined organics were dried over magnesium sulfate, filtered and concentrated. The residue was dissolved in DCM (2.4 mL). Imidazole (0.090 g, 1.32 mmole) and tert-butyldimethylchlorosilane (0.196 g, 1.30 mmole) were added and the mixture was stirred at RT for 3 hours. Water (25 mL) was added and the mixture was extracted with DCM (3×10 mL). The combined organics were dried over magnesium sulfate, filtered and concentrated. The crude residue was purified by silica gel chromatography to provide methyl N-(2-(4-(1,4-dioxane-2-carboxamido)piperidin-1-yl)thiazole-4-carbonyl)-O-(tert-butyldimethylsilyl)-L-serinate as a thick, pale yellow oil (0.321 g, 49%). ¹H NMR (400 MHz, CDCl₃): δ 7.95 (d, J=8.8 Hz, 1H), 7.39 (s, 1H), 6.48 (d, J=8.2 Hz, 1H), 4.79 (dt, J=8.9, 3.0 Hz, 1H), 4.18 (dd, J=4.9, 2.9 Hz, 2H), 4.12-4.07 (m, 1H), 4.01 (ddt, J=12.4, 8.6, 3.8 Hz, 2H), 3.97-3.90 (m, 1H), 3.90-3.83 (m, 2H), 3.83-3.71 (m, 2H), 3.76 (s, 3H), 3.66-3.55 (m, 1H), 3.42 (dd, J=11.5, 10.1 Hz, 1H), 3.23-3.10 (m, 2H), 2.03-1.96 (m, 2H), 1.63-1.49 (m, 2H), 0.88 (s, 9H), 0.05 (s, 3H), 0.03 (s, 3H).

### Methyl N—(N-(2-(4-(1,4-dioxane-2-carboxamido)piperidin-1-yl)thiazole-4-carbonyl)-O-(tert-butyldimethylsilyl)-L-seryl)-O-acetyl-L-serinate (58.2)

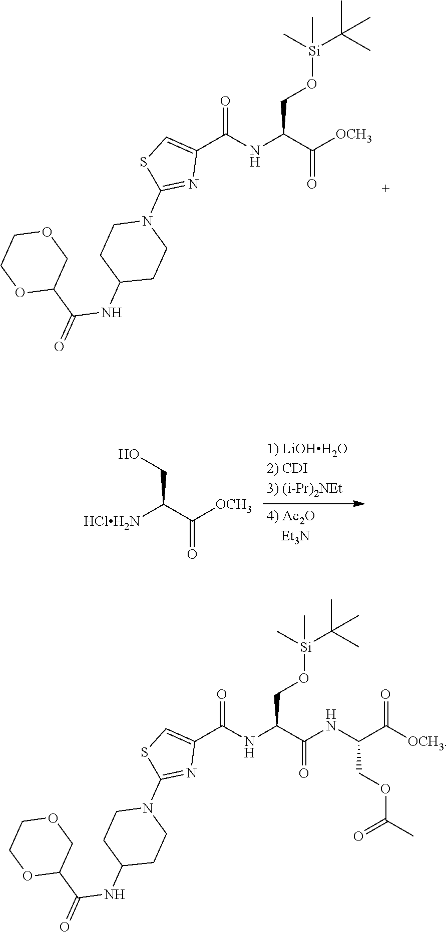

Methyl N-(2-(4-(1,4-dioxane-2-carboxamido)piperidin-1-yl)thiazole-4-carbonyl)-O-(tert-butyldimethylsilyl)-L-serinate (0.321 g, 0.577 mmole) was dissolved in 4/1/1 THF/methanol/water (2.4 mL). Lithium hydroxide monohydrate (0.049 g, 1.17 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in THE (2.4 mL). CDI (0.103 g, 0.635 mmole) was added portionwise and the mixture was stirred at RT for 60 minutes. L-Serine methyl ester hydrochloride (0.124 g, 0.797 mmole) was added followed by N,N-diisopropylethylamine (0.130 mL, 0.746 mmole). The mixture was stirred at RT for 18 hours and water (25 mL) was added. The mixture was extracted with ethyl acetate (3×10 mL) and the combined organics were dried over magnesium sulfate, filtered and concentrated. The residue was dissolved in DCM (2.4 mL). Triethylamine (0.090 mL, 0.642 mmole) and acetic anhydride (0.060 mL, 0.635 mmole) were added and the solution was stirred at RT for 18 hours. Water (25 mL) was added and the mixture was extracted with DCM (3×10 mL). The combined organics were dried over magnesium sulfate, filtered and concentrated. The crude residue was purified by silica gel chromatography to provide methyl N—(N-(2-(4-(1,4-dioxane-2-carboxamido)piperidin-1-yl)thiazole-4-carbonyl)-O-(tert-butyldimethylsilyl)-L-seryl)-O-acetyl-L-serinate as a thick, pale yellow oil (0.197 g, 50%). ¹H NMR (400 MHz, CDCl₃): δ 7.98 (d, J=7.1 Hz, 1H), 7.44 (d, J=7.8 Hz, 1H), 7.40 (d, J=2.5 Hz, 1H), 6.48 (d, J=8.2 Hz, 1H), 4.87 (dq, J=8.4, 4.6, 4.1 Hz, 1H), 4.58 (td, J=7.3, 3.6 Hz, 1H), 4.47 (ddd, J=11.1, 9.0, 4.0 Hz, 1H), 4.32 (dd, J=11.4, 3.7 Hz, 1H), 4.22-4.14 (m, 2H), 4.11 (dd, J=7.1, 2.9 Hz, 1H), 4.07-3.90 (m, 3H), 3.90-3.78 (m, 2H), 3.76 (s, 3H), 3.74-3.69 (m, 2H), 3.60 (td, J=11.5, 3.1 Hz, 1H), 3.42 (dd, J=11.5, 10.1 Hz, 1H), 3.24-3.09 (m, 2H), 2.06-1.97 (m, 2H), 2.02 (s, 3H), 1.65-1.48 (m, 2H), 0.92 (s, 9H), 0.14 (s, 3H), 0.12 (s, 3H).

### Methyl 2-(2-(2-(4-(1,4-dioxane-2-carboxamido)piperidin-1-yl)thiazole-4-carboxamido)acrylamido)acrylate (58)

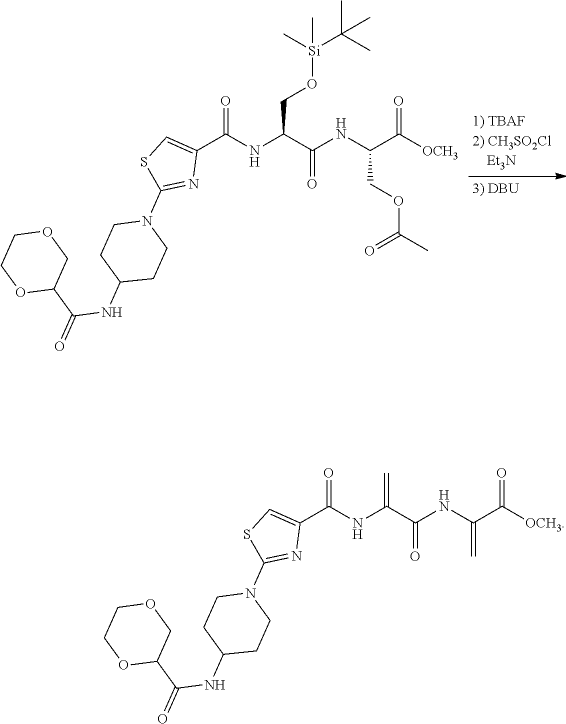 [SMILES: COC(=O)[C@H](COC(C)=O)NC(=O)[C@H](CO[Si](C)(C)C(C)(C)C)NC(=O)c1csc(N2CCC(NC(=O)C3COCCO3)CC2)n1 ; C=C(NC(=O)c1csc(N2CCC(NC(=O)C3COCCO3)CC2)n1)C(=O)NC(=C)C(=O)OC]

Methyl N—(N-(2-(4-(1,4-dioxane-2-carboxamido)piperidin-1-yl)thiazole-4-carbonyl)-O-(tert-butyldimethylsilyl)-L-seryl)-O-acetyl-L-serinate (0.197 g, 0.287 mmole) was dissolved in THF (2 mL). TBAF (1M solution in THF, 0.720 mL, 0.720 mmole) was added and the solution was stirred at RT for 18 hours. Water (25 mL) and sat. aq. sodium chloride (15 mL) were added and the mixture was extracted with ethyl acetate (3×10 mL). The combined organics were dried over magnesium sulfate, filtered and concentrated. The residue was dissolved in DCM (2 mL) and cooled to 0° C. Triethylamine (0.060 mL, 0.428 mmole) and methanesulfonyl chloride (0.034 mL, 0.439 mmole) were added and the mixture was stirred at 0° for 60 minutes. Water (25 mL) was added and the mixture was extracted with DCM (3×10 mL). The combined organics were dried over magnesium sulfate, filtered and concentrated. The residue was dissolved in THE (2 mL) and cooled to 0° C. DBU was added and the mixture was stirred at 0° for 60 minutes. Water (25 mL) and sat. aq. sodium chloride (15 mL) were added and the mixture was extracted with ethyl acetate (3×10 mL). The combined organics were dried over magnesium sulfate, filtered and concentrated. The crude residue was purified by silica gel chromatography to provide methyl 2-(2-(2-(4-(1,4-dioxane-2-carboxamido)piperidin-1-yl)thiazole-4-carboxamido)acrylamido)acrylate as a white solid (0.047 g, 33%). ¹H NMR (400 MHz, CDCl₃): δ 9.71 (s, 1H), 8.51 (s, 1H), 7.44 (s, 1H), 6.72 (d, J=2.1 Hz, 1H), 6.66 (s, 1H), 6.48 (d, J=8.2 Hz, 1H), 6.01 (d, J=1.3 Hz, 1H), 5.44 (t, J=1.9 Hz, 1H), 4.14 (ddd, J=22.7, 10.8, 3.1 Hz, 2H), 4.02 (dddd, J=22.7, 11.8, 5.6, 2.6 Hz, 3H), 3.89 (s, 3H), 3.86-3.71 (m, 3H), 3.66-3.53 (m, 1H), 3.42 (dd, J=11.5, 10.1 Hz, 1H), 3.20 (ddd, J=13.4, 11.6, 3.0 Hz, 2H), 2.10-1.98 (m, 2H), 1.65-1.51 (m, 2H).

### Compound 59: Methyl 2-(2-(2-(4-(tetrahydro-2H-pyran-4-carbonyl)piperazin-1-yl)thiazole-4-carboxamido)acrylamido)acrylate

### Methyl O-(tert-butyldimethylsilyl)-N-(2-(4-(tetrahydro-2H-pyran-4-carbonyl)piperazin-1-yl)thiazole-4-carbonyl)-L-serinate (59.1)

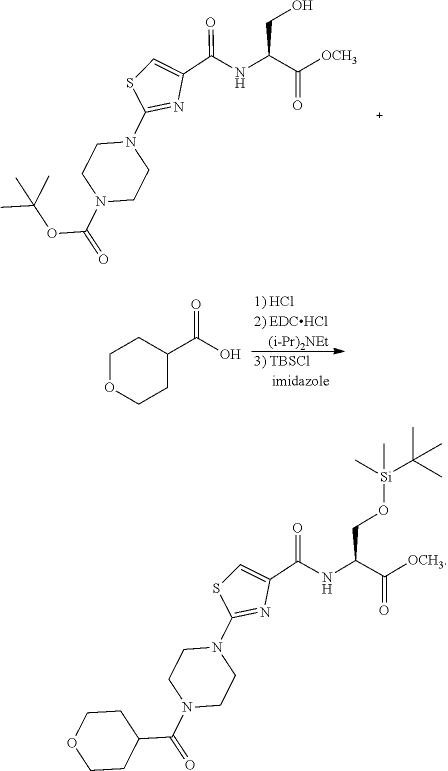 [SMILES: COC(=O)[C@H](CO)NC(=O)c1csc(N2CCN(C(=O)OC(C)(C)C)CC2)n1 ; O=C(O)C1CCOCC1 ; COC(=O)[C@H](CO[Si](C)(C)C(C)(C)C)NC(=O)c1csc(N2CCN(C(=O)C3CCOCC3)CC2)n1]

Tert-butyl (S′)-4-(4-((3-hydroxy-1-methoxy-1-oxopropan-2-yl)carbamoyl)thiazol-2-yl)piperazine-1-carboxylate (Example 47.2, 0.749 g, 1.81 mmole) was dissolved in DCM (3.6 mL). HCl (4M solution in 1,4-dioxane, 1.80 mL, 7.20 mmole) was added and the mixture was stirred at RT for 2 hours, then was concentrated. The residue was suspended in DCM (3.6 mL) and tetrahydro-2H-pyran-4-carboxylic acid (0.315 g, 2.42 mmole) was added. EDC·HCl (0.455 g, 2.37 mmole) was added, followed by N,N-diisopropylethyl amine (0.630 mL, 3.62 mmole) and the mixture was stirred at RT for 2 days. Water (25 mL) was added and the mixture was extracted with DCM (3×10 mL). The combined organics were dried over magnesium sulfate, filtered and concentrated. The residue was dissolved in DCM (3.6 mL) and imidazole (0.135 g, 1.98 mmole) was added, followed by tert-butyldimethylchlorosilane (0.300 g, 1.99 mmole). The mixture was stirred at RT for 18 hours and water (25 mL) was added. The mixture was extracted with DCM (3×10 mL) and the combined organics were dried over magnesium sulfate, filtered and concentrated. The crude residue was purified by silica gel chromatography to provide methyl 0-(tert-butyldimethylsilyl)-N-(2-(4-(tetrahydro-2H-pyran-4-carbonyl)piperazin-1-yl)thiazole-4-carbonyl)-L-serinate as a thick, pale yellow oil (0.543 g, 55%). ¹H NMR (400 MHz, CDCl₃): δ 7.93 (d, J=8.7 Hz, 1H), 7.45 (s, 1H), 4.79 (dt, J=8.7, 3.0 Hz, 1H), 4.16 (dd, J=8.3, 1.8 Hz, 1H), 4.05 (ddd, J=11.8, 4.3, 2.3 Hz, 2H), 3.90 (dd, J=10.0, 3.3 Hz, 1H), 3.82-3.71 (m, 2H), 3.77 (s, 3H), 3.66 (s, 2H), 3.59-3.42 (m, 6H), 2.76 (tt, J=11.3, 3.8 Hz, 1H), 2.02-1.88 (m, 2H), 1.67-1.59 (m, 2H), 0.88 (s, 9H), 0.05 (s, 3H), 0.03 (s, 3H).

### Methyl O-acetyl-N-(0-(tert-butyldimethylsilyl)-N-(2-(4-(tetrahydro-2H-pyran-4-carbonyl)piperazin-1-yl)thiazole-4-carbonyl)-L-seryl)-L-serinate (59.2)

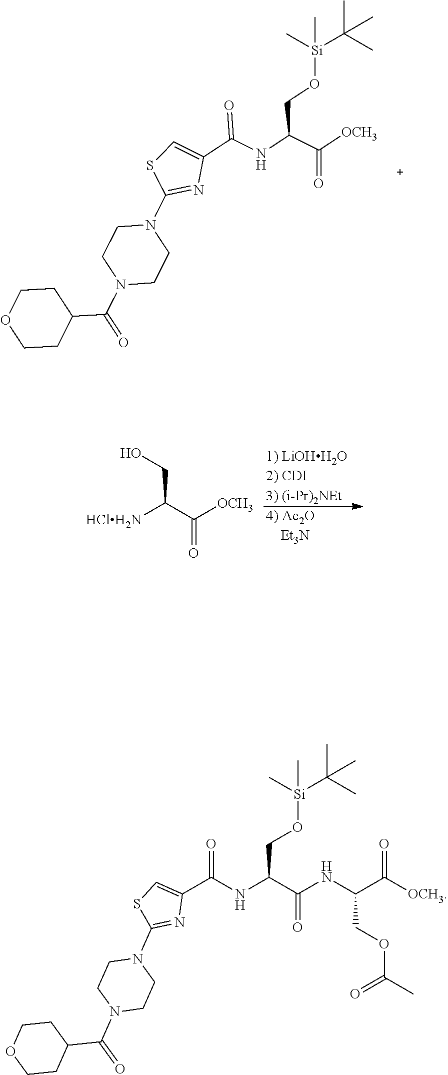

Methyl O-(tert-butyldimethylsilyl)-N-(2-(4-(tetrahydro-2H-pyran-4-carbonyl)piperazin-1-yl)thiazole-4-carbonyl)-L-serinate (0.543 g, 1.00 mmole) was dissolved in 4/1/1 THF/methanol/water (2 mL). Lithium hydroxide monohydrate (0.069 g, 1.54 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in THF (2 mL) and CDI (0.172 g, 1.06 mmole) was added portionwise (gas evolution was observed). The solution was stirred at RT for 60 minutes and L-serine methyl ester hydrochloride (0.205 g, 1.32 mmole) was added, followed by N,N-diisopropylethyl amine (0.230 mL, 1.32 mmole). The solution was stirred at RT for 18 hours and water (25 mL) was added. The mixture was extracted with ethyl acetate (3×10 mL) and the combined organics were dried over magnesium sulfate, filtered and concentrated. The residue was dissolved in DCM (2 mL) and triethylamine (0.155 mL, 1.11 mmole) and acetic anhydride (0.105 mL, 1.11 mmole) were added. The solution was stirred at RT for 18 hours and water (25 mL) was added. The mixture was extracted with DCM (3×10 mL) and the combined organics were dried over magnesium sulfate, filtered and concentrated. The crude residue was purified by silica gel chromatography (Isco CombiPrep, 12 g Redi Sep column, 70-100% ethyl acetate/hexane gradient)

### Methyl 2-(2-(2-(4-(tetrahydro-2H-pyran-4-carbonyl)piperazin-1-yl)thiazole-4-carboxamido)acrylamido)acrylate (59)

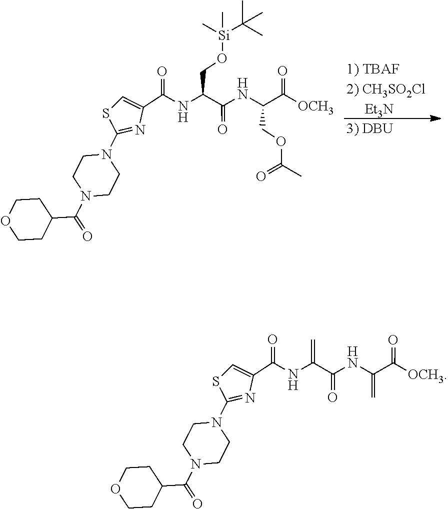 [SMILES: COC(=O)[C@H](COC(C)=O)NC(=O)[C@H](CO[Si](C)(C)C(C)(C)C)NC(=O)c1csc(N2CCN(C(=O)C3CCOCC3)CC2)n1 ; C=C(NC(=O)c1csc(N2CCN(C(=O)C3CCOCC3)CC2)n1)C(=O)NC(=C)C(=O)OC]

Methyl O-acetyl-N-(0-(tert-butyldimethylsilyl)-N-(2-(4-(tetrahydro-2H-pyran-4-carbonyl)piperazin-1-yl)thiazole-4-carbonyl)-L-seryl)-L-serinate (0.353 g, 0.527 mmole) was dissolved in THF. TBAF (1M solution in THF, 1.30 mL, 1.30 mmole) was added and the solution was stirred at RT for 18 hours. Water (25 mL) and sat. aq. sodium chloride (15 mL) were added and the mixture was extracted with ethyl acetate (3×10 mL). The combined organics were dried over magnesium sulfate, filtered and concentrated. The residue was dissolved in DCM (2 mL) and cooled to 0° C. Triethylamine 0.110 mL, 0.785 mmole) and methanesulfonyl chloride (0.061 mL, 0.788 mmole) were added and the mixture was stirred at 0° for 60 minutes. Water (25 mL) was added and the mixture was extracted with DCM (3×10 mL). The combined organics were dried over magnesium sulfate, filtered and concentrated. The residue was dissolved in THF (2 mL) and cooled to 0° C. DBU (0.120 mL, 0.802 mmole) was added and the mixture was stirred at 0° for 60 minutes. Water (25 mL) and sat. aq. sodium chloride (15 mL) were added and the mixture was extracted with ethyl acetate (3×10 mL). The combined organics were dried over magnesium sulfate, filtered and concentrated. The crude residue was purified by silica gel chromatography to provide methyl 2-(2-(2-(4-(tetrahydro-2H-pyran-4-carbonyl)piperazin-1-yl)thiazole-4-carboxamido)acrylamido)acrylate as a white solid (0.097 g, 39%). ¹H NMR (400 MHz, CDCl₃): δ 9.79-9.69 (m, 1H), 8.53 (s, 1H), 7.49 (s, 1H), 6.73 (d, J=2.2 Hz, 1H), 6.65 (s, 1H), 6.01 (d, J=1.2 Hz, 1H), 5.45 (t, J=1.9 Hz, 1H), 4.05 (ddd, J=11.7, 4.3, 2.2 Hz, 2H), 3.90 (s, 3H), 3.79 (s, 2H), 3.73-3.58 (m, 4H), 3.47 (td, J=11.8, 2.1 Hz, 4H), 2.76 (tt, J=11.3, 3.8 Hz, 1H), 1.95 (dtd, J=13.7, 11.7, 4.4 Hz, 2H), 1.67-1.61 (m, 2H).

### Compound 60: Tert-butyl (R)-4-(4-((3-((3-methoxy-3-oxoprop-1-en-2-yl)amino)-3-oxoprop-1-en-2-yl)carbamoyl)thiazol-2-yl)-2-methylpiperazine-1-carboxylate

### Ethyl (R)-2-(4-(tert-butoxycarbonyl)-3-methylpiperazin-1-yl)thiazole-4-carboxylate (60.1)

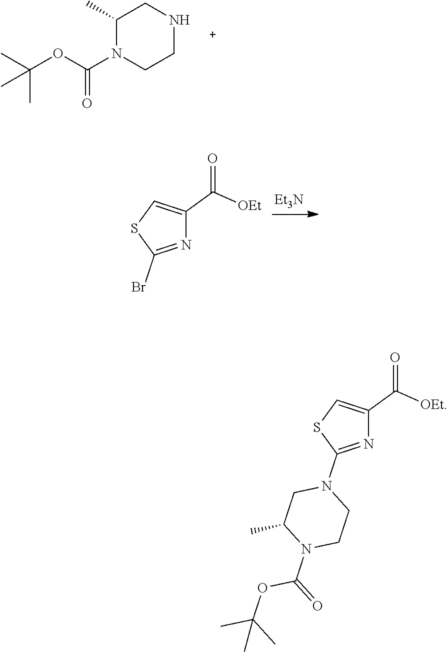 [SMILES: "Et3N" ; C[C@@H]1CNCCN1C(=O)OC(C)(C)C ; CCOC(=O)c1csc(Br)n1 ; CCOC(=O)c1csc(N2CCN(C(=O)OC(C)(C)C)[C@H](C)C2)n1]

Tert-butyl (R)-2-methylpiperazine-1-carboxylate (1.499 g, 7.48 mmole) was dissolved in DMA (7.5 mL). Ethyl 2-bromothiazole (1.780 g, 7.54 mmole) was added followed by triethylamine (1.30 mL, 9.28 mmole). The mixture was heated to 80° C. for 28 hours, then was cooled to RT and diluted with ethyl acetate (25 mL). The mixture was washed with water (3×25 mL) and the organics were dried over magnesium sulfate, filtered and concentrated. The crude residue was purified by silica gel chromatography to provide ethyl (R)-2-(4-(tert-butoxycarbonyl)-3-methylpiperazin-1-yl)thiazole-4-carboxylate as a thick, orange gel (1.515 g, 57%). ¹H NMR (400 MHz, CDCl₃): δ 7.45 (s, 1H), 4.42-4.35 (m, 1H), 4.35 (q, J=7.1 Hz, 2H), 4.03-3.90 (m, 2H), 3.68 (dt, J=12.8, 1.9 Hz, 1H), 3.34 (dd, J=12.7, 4.1 Hz, 1H), 3.24 (ddd, J=13.3, 11.9, 3.5 Hz, 1H), 3.09 (td, J=12.1, 3.6 Hz, 1H), 1.48 (s, 9H), 1.37 (t, J=7.1 Hz, 3H), 1.23 (d, J=6.8 Hz, 3H)

### Tert-butyl (R)-4-(4-(((S)-3-((tert-butyldimethylsilyl)oxy)-1-methoxy-1-oxopropan-2-yl)carbamoyl)thiazol-2-yl)-2-methylpiperazine-1-carboxylate (60.2)

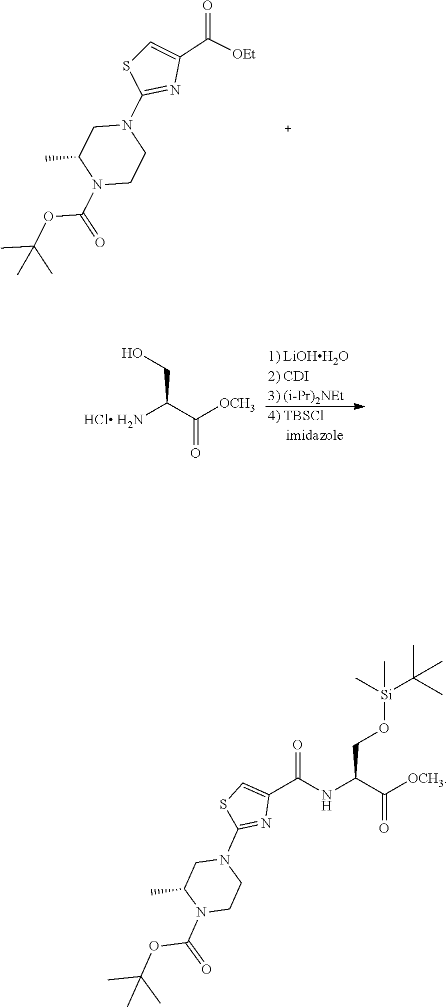

Ethyl (R)-2-(4-(tert-butoxycarbonyl)-3-methylpiperazin-1-yl)thiazole-4-carboxylate (0.605 g, 1.70 mmole) was dissolved in 4/1/1 THF/methanol/water (3.4 mL). Lithium hydroxide monohydrate (0.143 g, 3.41 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in THE (3.4 mL). CDI (0.291 g, 1.79 mmole) was added portionwise (gas evolution was observed) and the solution was stirred at RT for 45 minutes. L-Serine methyl ester hydrochloride (0.341 g, 2.19 mmole) was added followed by N,N-diisopropylethylamine (0.390 mL, 2.24 mmole) and the mixture was stirred at RT for 18 hours. Water (25 mL) was added and the mixture was extracted with ethyl acetate (3×10 mL). The combined organics were dried over magnesium sulfate, filtered and concentrated. The residue was dissolved in DCM (3.4 mL), then imidazole (0.129 g, 1.89 mmole) and tert-butyldimethylchlorosilane (0.280 g, 1.86 mmole) were added. The mixture was stirred at RT for 3 hours and water (25 mL) was added. The mixture was extracted with DCM (3×10 mL) and the combined organics were dried over magnesium sulfate, filtered and concentrated. The crude residue was purified by silica gel chromatography to provide tert-butyl (R)-4-(4-(((S)-3-((tert-butyldimethylsilyl)oxy)-1-methoxy-1-oxopropan-2-yl)carbamoyl)thiazol-2-yl)-2-methylpiperazine-1-carboxylate as a yellow oil (0.797 g, 86%). ¹H NMR (400 MHz, CDCl₃): δ 7.94 (d, J=8.7 Hz, 1H), 7.39 (s, 1H), 4.82-4.74 (m, 1H), 4.38 (s, 1H), 4.22-4.13 (m, 2H), 4.00-3.85 (m, 4H), 3.77 (d, J=5.0 Hz, 4H), 3.68 (dt, J=12.8, 1.8 Hz, 1H), 3.30 (dd, J=12.8, 4.1 Hz, 1H), 3.23 (ddd, J=13.3, 11.9, 3.5 Hz, 1H), 3.07 (td, J=12.1, 3.7 Hz, 1H), 1.49 (s, 9H), 1.26-1.21 (m, 3H), 0.88 (s, 9H), 0.05 (s, 3H), 0.03 (s, 3H)

### Tert-butyl (R)-4-(4-(((6S,9S)-9-(methoxycarbonyl)-2,2,3,3-tetramethyl-7,12-dioxo-4,11-dioxa-8-aza-3-silatridecan-6-yl)carbamoyl)thiazol-2-yl)-2-methylpiperazine-1-carboxylate (60.3)

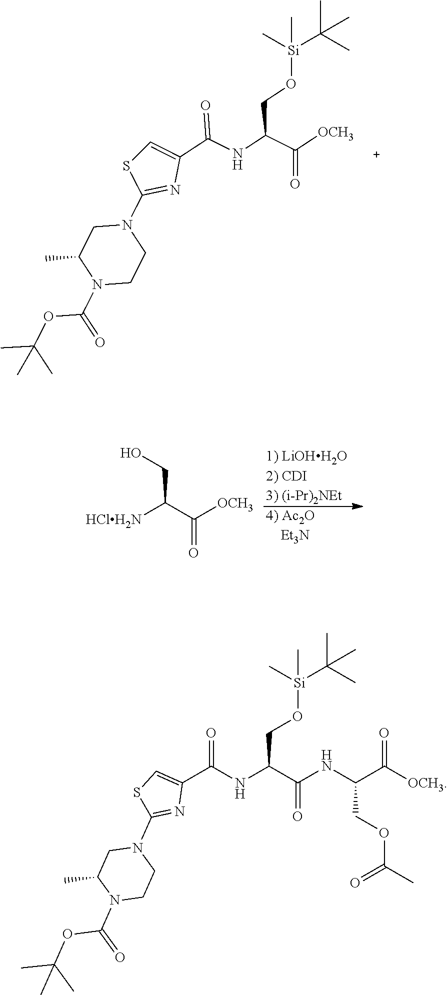

Tert-butyl (R)-4-(4-(((S)-3-((tert-butyldimethylsilyl)oxy)-1-methoxy-1-oxopropan-2-yl)carbamoyl)thiazol-2-yl)-2-methylpiperazine-1-carboxylate (0.797 g, 1.47 mmole) was dissolved in 4/1/1 THF/methanol/water (3 mL). Lithium hydroxide monohydrate (0.131 g, 3.12 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was suspended in THF (3 mL). CDI (0.251 g, 1.55 mmole) was added portionwise (gas evolution was observed) and the solution was stirred at RT for 45 minutes. L-Serine methyl ester hydrochloride (0.296 g, 1.90 mmole) was added followed by N,N-diisopropylethylamine (0.330 mL, 1.89 mmole) and the mixture was stirred at RT for 18 hours. Water (25 mL) was added and the mixture was extracted with ethyl acetate (3×10 mL). The combined organics were dried over magnesium sulfate, filtered and concentrated. The residue was dissolved in DCM (3 mL), then triethylamine (0.230 mL, 1.64 mmole) and acetic anhydride (0.60 mL, 1.69 mmole) were added. The mixture was stirred at RT for 3 hours and water (25 mL) was added. The mixture was extracted with DCM (3×10 mL) and the combined organics were dried over magnesium sulfate, filtered and concentrated. The crude residue was purified by silica gel chromatography to provide tert-butyl (R)-4-(4-(((6S,9S)-9-(methoxycarbonyl)-2,2,3,3-tetramethyl-7,12-dioxo-4,11-dioxa-8-aza-3-silatridecan-6-yl)carbamoyl)thiazol-2-yl)-2-methylpiperazine-1-carboxylate as a thick, pale yellow oil (0.463 g, 47%). ¹H NMR (400 MHz, CDCl₃): δ 7.97 (d, J=7.0 Hz, 1H), 7.44 (d, J=7.7 Hz, 1H), 7.41 (s, 1H), 4.88 (dt, J=7.7, 3.8 Hz, 1H), 4.58 (tt, J=6.7, 3.3 Hz, 1H), 4.48 (dd, J=11.3, 3.9 Hz, 1H), 4.39 (t, J=5.8 Hz, 1H), 4.32 (dd, J=11.4, 3.7 Hz, 1H), 4.19 (dd, J=9.8, 3.6 Hz, 1H), 3.93 (dd, J=18.1, 13.4 Hz, 2H), 3.82 (d, J=14.0 Hz, 1H), 3.76 (d, J=3.4 Hz, 3H), 3.75-3.71 (m, 1H), 3.68 (dd, J=13.2, 6.2 Hz, 1H), 3.35-3.18 (m, 2H), 3.07 (tt, J=12.3, 6.6 Hz, 1H), 2.03 (d, J=1.2 Hz, 3H), 1.49 (s, 9H), 1.25-1.20 (m, 4H), 0.93 (s, 9H), 0.15 (s, 3H), 0.13 (s, 3H)

### Tert-butyl (R)-4-(4-((3-((3-methoxy-3-oxoprop-1-en-2-yl)amino)-3-oxoprop-1-en-2-yl)carbamoyl)thiazol-2-yl)-2-methylpiperazine-1-carboxylate (60)

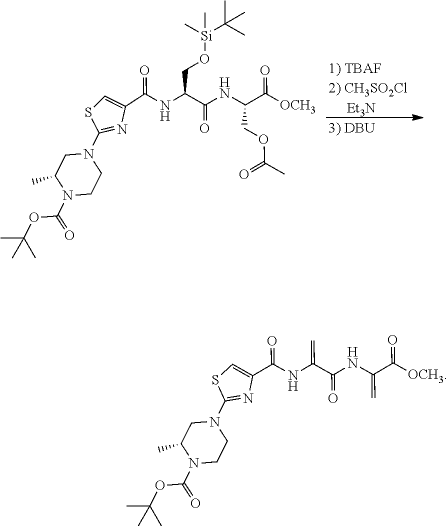 [SMILES: COC(=O)[C@H](COC(C)=O)NC(=O)[C@H](CO[Si](C)(C)C(C)(C)C)NC(=O)c1csc(N2CCN(C(=O)OC(C)(C)C)[C@H](C)C2)n1 ; C=C(NC(=O)c1csc(N2CCN(C(=O)OC(C)(C)C)[C@H](C)C2)n1)C(=O)NC(=C)C(=O)OC]

Tert-butyl (R)-4-(4-(((6S,9S)-9-(methoxycarbonyl)-2,2,3,3-tetramethyl-7,12-dioxo-4,11-dioxa-8-aza-3-silatridecan-6-yl)carbamoyl)thiazol-2-yl)-2-methylpiperazine-1-carboxylate (0.463 g, 0.689 mmole) was dissolved in THE (2 mL). TBAF (1M solution in THF, 1.70 mL, 1.70 mmole) was added and the solution was stirred at RT for 24 hours. Water (25 mL) and sat. aq. sodium chloride (15 mL) were added and the mixture was extracted with ethyl acetate (3×10 mL). The combined organics were dried over magnesium sulfate, filtered and concentrated. The residue was dissolved in DCM (2 mL) and cooled to 0° C. Triethylamine (0.150 mL, 1.07 mmole) and methanesulfonyl chloride (0.080 mL, 1.03 mmole) were added and the mixture was stirred at 0° for 60 minutes. Water (25 mL) was added and the mixture was extracted with DCM (3×10 mL). The combined organics were dried over magnesium sulfate, filtered and concentrated. The residue was dissolved in THE (2 mL) and cooled to 0° C. DBU (0.155 mL, 1.04 mmole) was added and the mixture was stirred at 0° for 60 minutes. Water (25 mL) and sat. aq. sodium chloride (15 mL) were added and the mixture was extracted with ethyl acetate (3×10 mL). The combined organics were dried over magnesium sulfate, filtered and concentrated. The crude residue was purified by silica gel chromatography to provide tert-butyl (R)-4-(4-((3-((3-methoxy-3-oxoprop-1-en-2-yl)amino)-3-oxoprop-1-en-2-yl)carbamoyl)thiazol-2-yl)-2-methylpiperazine-1-carboxylate as a white solid (0.150 g, 45%). ¹H NMR (400 MHz, CDCl₃): δ 9.72 (s, 1H), 8.51 (s, 1H), 7.44 (s, 1H), 6.72 (d, J=2.1 Hz, 1H), 6.67 (s, 1H), 6.01 (d, J=1.2 Hz, 1H), 5.44 (t, J=1.9 Hz, 1H), 4.41 (s, 1H), 3.98 (ddd, J=12.5, 7.9, 5.5 Hz, 2H), 3.89 (s, 3H), 3.70 (dt, J=12.8, 1.8 Hz, 1H), 3.32 (dd, J=12.8, 4.0 Hz, 1H), 3.24 (ddd, J=13.6, 11.9, 3.6 Hz, 1H), 3.11 (td, J=12.2, 3.8 Hz, 1H), 1.49 (s, 9H), 1.25 (dd, J=6.9, 1.5 Hz, 3H).

The following Compounds 61-65 are synthesized with procedures similar to those provided above.

### Compound 61: Methyl 2-(2-(2-(4-((3-methoxypropyl)carbamoyl)piperidin-1-yl)thiazole-4-carboxamido)acrylamido)acrylate

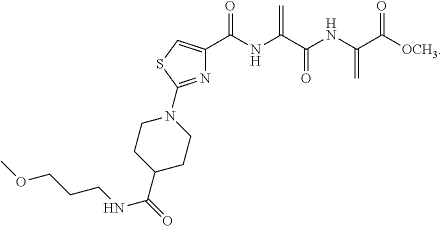 [SMILES: C=C(NC(=O)c1csc(N2CCC(C(=O)NCCCOC)CC2)n1)C(=O)NC(=C)C(=O)OC]

### Compound 62: Methyl 2-(2-(2-(4-(2-((3-methoxypropyl)amino)-2-oxoethyl)piperidin-1-yl)thiazole-4-carboxamido)acrylamido)acrylate

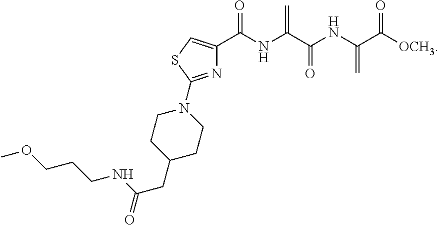 [SMILES: C=C(NC(=O)c1csc(N2CCC(CC(=O)NCCCOC)CC2)n1)C(=O)NC(=C)C(=O)OC]

### Compound 63: Methyl 2-(2-(2-(4-(3-methoxypropyl)-3-oxopiperazin-1-yl)thiazole-4-carboxamido)acrylamido)acrylate

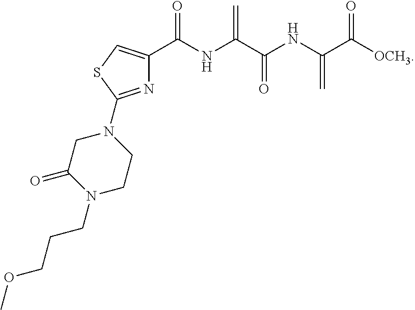 [SMILES: C=C(NC(=O)c1csc(N2CCN(CCCOC)C(=O)C2)n1)C(=O)NC(=C)C(=O)OC]

### Compound 64: Methyl (S)-2-(2-(2-(3-(tetrahydro-2H-pyran-4-carboxamido)pyrrolidin-1-yl)thiazole-4-carboxamido)acrylamido)acrylate

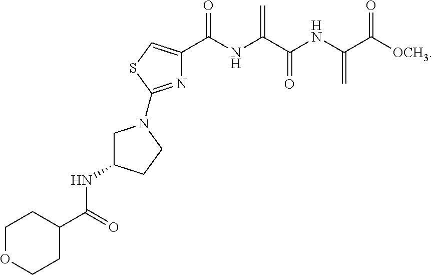 [SMILES: C=C(NC(=O)c1csc(N2CC[C@H](NC(=O)C3CCOCC3)C2)n1)C(=O)NC(=C)C(=O)OC]

### Compound 65: Methyl (R)-2-(2-(2-(3-(tetrahydro-2H-pyran-4-carboxamido)pyrrolidin-1-yl)thiazole-4-carboxamido)acrylamido)acrylate

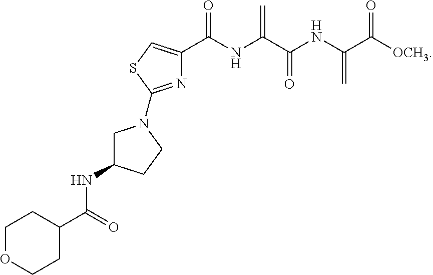 [SMILES: C=C(NC(=O)c1csc(N2CC[C@@H](NC(=O)C3CCOCC3)C2)n1)C(=O)NC(=C)C(=O)OC]

### Assay Description and Protocols

### Description of Activity Assays to Measure Prx3 Inhibition

The compounds described herein generally react with two essential cysteine residues in the mitochondrial peroxidase PRX3, leading to the formation of a non-reducible, covalent crosslink of two PRX3 monomers. This crosslink inactivates PRX3. Increased PRX3 crosslink is associated with mitochondrial stress, increased cell death in cell models of malignant mesothelioma and is associated with decreased tumor volume in a mouse model. Cunniff et al. (2015) PloS one 10, e0127310.

PRX3 uses an essential reduced cysteine residue to reduce hydrogen peroxide. During this process, PRX3 becomes oxidized forming a reversible disulfide bond that links two PRX3 monomers. In the cell, this disulfide can be reduced by the combined activity of thioredoxin 2 (TRX2), thioredoxin reductase 2, and NADPH. The disulfide can also be reduced by small molecule reductants such as dithiothreitol (DTT). In contrast, the compound-crosslink with PRX3 is irreversible and cannot be broken by the addition of reductants.

To test the activity of compounds, two complimentary assays were used as described below. The Biochemical PRX3 Inhibition Assay tests the ability of each compound to crosslink PRX3 in a simple, in vitro system. In the Cellular Activity Assay, we are testing the ability of compounds to kill SK—OV-3 ovarian cancer cells.

### Biochemical Prx3 Inhibition Assay

This assay tests the ability of each compound to form a covalent adduct with PRX3. The assay is performed as described in Nelson et al. (2021) Antioxidants (Basel) 10, 150; and Cunniff et al. (2015) PloS one 10, e0127310, and follows the appearance of non-reducible PRX3 crosslinks by SDS PAGE gel electrophoresis and follows the appearance of single PRX3 adducts by mass spectrometry. For this assay, purified human PRX3, hydrogen peroxide (the PRX3 substrate) and all the components required to enable PRX3 to catalytically cycle (thioredoxin-thioredoxin reductase-NAPDH system) are included (details below).

The biochemical PRX3 Inhibition assay contains 100 μM PRX3, 50 μM human TRX2, 0.5 μM mouse thioredoxin reductase, and a NADPH regenerating system composed of 3.2 mM glucose 6-phosphate, 3.2 U/ml glucose 6-phosphate dehydrogenase and 0.4 mM NADPH. Samples are incubated for 1-2 hr at 37° C. with either 0.2 mM TS (positive control), compounds or an equivalent volume of DMSO (negative control). During this incubation, hydrogen peroxide is added to induce turnover of PRX3. Reactions are stopped by the addition of a buffer containing 100 mM dithiothreitol (to break disulfide bonds) and SDS (detergent to denature proteins). NADPH, Glucose 6-Phosphate, and glucose 6-phosphate dehydrogenase were purchased from Sigma Aldrich. PRX3, thioredoxin, and thioredoxin reductase were all purified to >98% purity in the Lowther laboratory according to protocols referenced in Nelson et al. (2021) Antioxidants (Basel) 10, 150; and Cunniff et al. (2015) PloS one 10, e0127310.

To measure the amount of PRX3 crosslink, proteins in the reaction were separated by SDS-polyacrylamide gel electrophoresis and stained for total protein using GelCode Blue (Life Technologies). The amount of unmodified PRX3 and TS-PRX3 crosslink was measured by densiometric analysis of the signal for the PRX3 band running at the MW of a PRX3 crosslink (˜46 kDa) compared to the PRX3 signal at the MW of the un-modified PRX3 (−23 kD).

To measure covalent adduct formation on a single PRX3 monomer, each reaction was exchanged into a mass spectrometry compatible buffer containing 40 mM ammonium citrate, pH 8.3 made in HPLC water. Sample was mixed 1:1 with a matrix solution containing 30 mg/mL sinapinic acid in 70% (vol/vol) acetonitrile, 0.2% formic acid and spotted to onto the sample plate. PRX3 mass was measured by MALDI-TOF MS analysis on a Bruker Daltonics MALDI-TOF MS spectrometer. Spectra were analyzed in FLEXAnalysis Software. The intensity of the reduced PRX3 peak (SH) and intensity of the analog adduct peak was determined and corrected for background signal at the adduct peak in the DMSO control. The fraction of single analog adduct in the monomer peak was determined by dividing the intensity of the adduct peak by the summed intensity of the SH and adduct peaks.

### Cellular Activity Assay

The Cellular Activity Assay measures the EC₅₀ of each compound in human SK—OV-3 ovarian cancer cells TS to be taken into the cell, transported to the mitochondria, and crosslink PRX3.

The cytotoxicity of compounds was measured as described in Nelson et al. (2021) Antioxidants (Basel) 10, 150; Cunniff et al. (2015) PloS one 10, e0127310; and Newick et al. (2012) PloS one 7, e39404. SK—OV-3 is an adherent, epithelial, adenocarcinoma cell line obtained from ATCC (ref #: HTB-77). SK—OV-3 cells are resistant to the commonly used chemotherapeutics, cis-platinum and doxorubicin. For this PRX3 crosslinking assay, SK—OV-3 cells were plated in a 96-well plate. After 24 h recovery, the cells were treated for 48 hr with multiple concentrations of each compound ranging from 0.1-100 μM.

Cells were washed with PBS to remove dead cells and the remaining live cells were fixed with 3.0% formaldehyde and stained with 0.1% crystal violet. The amount of crystal violet was determined by reading the absorbance at 540 nm (crystal violet dye dissolved in 100% methanol) using a plate reader. The signal for crystal violet dye is proportional to the biomass of remaining live cells. GraphPad Prism9 software was used to calculate the effective inhibitory concentration (EC₅₀) of test compounds. Results for each compound, are normalized to the amount of cells in control wells treated with the equivalent concentration of DMSO (negative control). TS or Compound 4 were included in each set of assays as a positive control.

### Solubility Assay

A 20 mM stock solution was made for each compound in DMSO. To measure solubility, 0.03-0.05 mL of the stock was added to 0.27 mL 20 mM HEPES, 100 mM NaCl, pH 7.5. The solution was mixed and rotated 18-24 hours at ambient temperature. The next day, solutions were centrifuged for 10 min at 20,000×g at 24 HC to remove insoluble, solid material. Next, 100 μL of the supernatant containing the soluble compound was added to 900 μL DMSO and High Performance Liquid Chromatography was performed as described in Table 1 and either HIPLC gradient 1 (Table 2) or HIPLC gradient 2 (Table 3). For each analog, a 200 μM standard and a 20 μM standard were prepared and run in parallel (standards were dissolved completely in 10000 DM). The amount ofLcompound in the experimental sample was calculated by comparing the area under the curve for compound peak to the area for the closest standard and correcting for dilution.

TABLE 1
HPLC Instrument Parameters for Solubility Assay

| Instrument | Agilent 1260 Infinity II HPLC System | |
|---|---|---|
| Software to Process | OpenLab CDS Chemstation Edition | |
| data | Software for LC and LC/MS Systems. | |
| | Rev. C.01.09 (144) from Agilent | |
| | Technologies | |
| Column | 100 mM Poroshell 120EC-C18 | |
| | 4.6 × 100 mm × 2.7 μm | |
| | Agilent Product #: 695975-9027. | |
| | Serial #: USCFS26515 | |
| | Batch #: B19136 | |
| Buffer A | Water 0.1% Trifluoroacetic acid | |
| | (TFA) | |
| Buffer B | Acetonitrile 0.1% TFA | |
| Flow Rate | 0.8 | mL/min |
| wavelength | 254 | nm |
| Col Temp | 30° | C. |
| Autosampler Temp | 23° | C. |
| Seal wash solvent | 50% Acetonitirle: 50% Water | |
| Needle wash solvent | 50% Acetonitrile: 50% Water | |
| Diluent | DMSO | |
| Sample Preparation | 0.1 mL sample to 0.9 mL DMSO | |
| Dilution Factor | 10 | |
| Injection vol | 10 | μl |
| Time | 14.1 | min |

TABLE 2
HPLC Gradient Program 1

| Time (min) | % H2O | % CH3CN | ml/min | max pressure |
|---|---|---|---|---|
| 0 | 70 | 30 | 1.0 | 400 |
| 2 | 0 | 98 | 1.0 | 400 |
| 8 | 0 | 98 | 1.0 | 400 |
| 10 | 70 | 30 | 1.0 | 400 |
| 11 | 70 | 30 | 1.0 | 400 |
| 14 | 70 | 30 | 1.0 | 400 |

TABLE 3
Alternate HPLC Gradient 2

| Time (min) | % H2O | % CH3CN | ml/min | max pressure |
|---|---|---|---|---|
| 0 | 70 | 30 | 1.0 | 400 |
| 2 | 0 | 98 | 1.0 | 400 |
| 8 | 0 | 98 | 1.0 | 400 |
| 10 | 70 | 30 | 1.0 | 400 |
| 11 | 70 | 30 | 1.0 | 400 |
| 14 | 70 | 30 | 1.0 | 400 |

### Antimicrobial Activity Assay

To determine the minimal inhibitory concentration (MIC) of TS with Enterococcus hirae (ATCC #10541), a frozen stock of E. hirae was streaked onto a plate containing BHI growth media (7.8 g/L brain extract, 2.0 g/L dextrose, 2.5 g/L disodium phosphate, 9.7 g/L heart extract, 10 g/L proteose peptone, 5 g/L sodium chloride supplemented with 0.01% (v/v) polysorbate 80, 10 g/mL dextrose, and 10 g/L agar. A single colony of E. hirae was used to inoculate 10 mL of media 41 (described in USP37<81>: 9 g/L pancreatic digest of casein, 5 g/L yeast extract, 20 g/L dextrose, 10 g/L sodium citrate, 1 g/L monobasic potassium phosphate, and 1 g/L dibasic potassium phosphate and grown overnight at 37° C. The overnight growth was then diluted into growth media to make a single solution of E. hirae with a final optical density at 600 nm=0.02 AU. A 20 mM stock solution of Thiostrepton in DMSO was made and used to make multiple dilutions of TS in DMSO. Each TS dilution (0.1 mL) was then added to a test tube containing 10 mL of the diluted E. hirae and grown at 37° C. overnight while shaking at 180 rpm. The next morning, each tube was visually inspected to determine if the cells grew. Final concentrations tested were 0.120, 0.090, 0.075, 0.060, 0.045, 0.030, and 0.015 μM TS. Complete growth was observed at TS concentrations <0.030 μM TS and no growth was observed at all TS concentrations >0.045 μM. Three replicates were made on three separate days (n=9). No intermediate growth was observed, and all replicates showed the same growth profile. For TS analogs, the dilute E. hirae was treated with 20 μM final concentration of each analog (three replicates each). No growth inhibition was observed for any analog. DMSO was used as a positive control (complete cell growth) and 20 μM thiostrepton was used as a negative control.

### Aqueous Stability Assay

To measure aqueous stability after 24 hours, samples were prepared and analyzed by HPLC as described for the Solubility Assay. % Purity was calculated for the control sample (compound in DMSO) and the assay sample (compound in aqueous buffer) according to the following equations

$$\% Purity = \frac{Area Compound Peak}{Total Area of all peaks} \times 100 .$$

Diluent peaks and solvent peaks are excluded from the calculation. Relative stability was calculated by dividing the Purity of the Assay Sample by the Purity of the Standard sample.

### Assay Results

TABLE 4
Key

| | | | PRX3 | | | | | |
|---|---|---|---|---|---|---|---|---|
| | | | Inhibition | | | | | |
| | | | (Crosslink + | | | | | |
| | | | Mono- | | | | Anti- | |
| | EC50 | | adduct) | Solubility | | Stability | microbial | |
| ++++ | <1 | μM | | | | | | |
| +++ | 1-10 | μM | >90% | >1 | mM | | 0.02-0.2 | μM |
| ++ | 10-25 | μM | 50-90% | 0.1-1 | mM | >0.95 | 0.2-2 | μM |
| | | | | | | after | | |
| | | | | | | 24 | | |
| | | | | | | hours | | |
| + | 25-100 | μM | 5-50% | <0.1 | mM | >0.90 | 2-20 | μM |
| | | | | | | after | | |
| | | | | | | 24 | | |
| | | | | | | hours | | |

| | | | | | | |
|---|---|---|---|---|---|---|
| − | >100 | μM | polymerized | <0.90 | >20 | μM |

| | |
|---|---|
| N.D. | not determined |

TABLE 4
Assay Results for Each Compound

| | | PRX3 | | Aqueous | Antimicrobial |
|---|---|---|---|---|---|
| Compound | EC₅₀ | Inhibition | Solubility | Stability | Activity |
| 1 | +++ | ++ | ++ | − | − |
| 2 | +++ | ++ | + | ++ | − |
| 3 | ++ | ++ | ++ | − | − |
| 4 | +++ | ++ | + | ++ | − |
| 5 | +++ | +++ | + | ++ | − |
| 6 | +++ | +++ | + | − | − |
| 7 | +++ | ++ | + | ++ | − |
| 8 | +++ | +++ | + | ++ | − |
| 9 | +++ | +++ | + | − | − |
| 10 | +++ | +++ | + | + | − |
| 11 | +++ | +++ | + | ++ | − |
| 12 | +++ | +++ | + | ++ | − |
| 13 | +++ | +++ | ++ | ++ | − |
| 14 | + | +++ | + | − | − |
| 15 | +++ | +++ | + | − | − |
| 16 | ++ | +++ | + | − | − |
| 17 | ++ | +++ | ++ | ++ | − |
| 18 | + | + | ++ | ++ | − |
| 19 | +++ | +++ | + | ++ | − |
| 20 | +++ | +++ | + | ++ | − |
| 21 | +++ | +++ | + | ++ | − |
| 22 | +++ | +++ | + | ++ | − |
| 23 | +++ | +++ | + | ++ | − |
| 24 | +++ | ++ | + | ++ | − |
| 25 | ++ | ++ | + | ++ | − |
| 26 | +++ | ++ | ++ | ++ | − |
| 27 | +++ | +++ | + | − | − |
| 28 | +++ | +++ | + | − | − |
| 29 | +++ | +++ | ++ | ++ | − |
| 30 | + | + | + | ++ | − |
| 31 | + | + | +++ | − | − |
| 32 | + | ++ | ++ | − | − |
| 33 | + | +++ | +++ | − | − |
| 34 | +++ | +++ | +++ | ++ | − |
| 35 | +++ | +++ | ++ | − | N.D. |
| 36 | +++ | +++ | + | − | N.D. |
| 37 | +++ | + | + | − | N.D. |
| 38 | +++ | ++ | + | + | N.D. |
| 39 | +++ | ++ | + | ++ | N.D. |
| 40 | ++ | +++ | ++ | + | N.D. |
| 41 | ++ | +++ | +++ | ++ | N.D. |
| 42 | +++ | +++ | + | ++ | N.D. |
| 43 | +++ | +++ | + | ++ | N.D. |
| 44 | +++ | +++ | + | ++ | N.D. |
| 45 | +++ | ++ | + | ++ | N.D. |
| 46 | +++ | +++ | ++ | ++ | N.D. |
| 47 | + | ++ | +++ | + | N.D. |
| 48 | +++ | ++ | + | ++ | N.D. |
| 49 | +++ | +++ | + | ++ | N.D. |
| 50 | +++ | + | + | ++ | N.D. |
| 51 | ++ | +++ | + | ++ | N.D. |
| 52 | ++ | +++ | + | ++ | N.D. |
| 53 | ++ | +++ | + | ++ | N.D. |
| 54 | + | ++ | + | ++ | N.D. |
| 55 | ++ | ++ | + | ++ | N.D. |
| 56 | +++ | ++ | + | ++ | N.D. |
| 57 | +++ | +++ | + | ++ | N.D. |
| 58 | N.D. | +++ | ++ | + | N.D. |
| 59 | N.D. | +++ | ++ | ++ | N.D. |
| 60 | N.D. | +++ | + | ++ | N.D. |

### REFERENCES

1. Cairns, R. A., Harris, I. S., And Mak, T. W. (2011) Regulation Of Cancer Cell Metabolism. Nat Rev Cancer 11, 85-95
2. Weinberg, F., and Chandel, N. S. (2009) Reactive oxygen species-dependent signaling regulates cancer. Cell Mol Life Sci 66, 3663-3673
3. Weinberg, F., and Chandel, N. S. (2009) Mitochondrial metabolism and cancer. Ann N Y Acad Sci 1177, 66-73
4. Weinberg, F., Hamanaka, R., Wheaton, W. W., Weinberg, S., Joseph, J., Lopez, M., Kalyanaraman, B., Mutlu, G. M., Budinger, G. R., and Chandel, N. S. (2010) Mitochondrial metabolism and ROS generation are essential for Kras-mediated tumorigenicity. Proc Natl Acad Sci USA 107, 8788-8793
5. Park, H. J., Carr, J. R., Wang, Z., Nogueira, V., Hay, N., Tyner, A. L., Lau, L. F., Costa, R. H., and Raychaudhuri, P. (2009) FoxM1, a critical regulator of oxidative stress during oncogenesis. EMBO J28, 2908-2918
6. Nonn, L., Berggren, M., and Powis, G. (2003) Increased expression of mitochondrial peroxiredoxin-3 (thioredoxin peroxidase-2) protects cancer cells against hypoxia and drug-induced hydrogen peroxide-dependent apoptosis. Mol Cancer Res 1, 682-689
7. Cox, A. G., Winterbourn, C. C., and Hampton, M. B. (2009) Mitochondrial peroxiredoxin involvement in antioxidant defence and redox signalling. Biochem J 425, 313-325
8. Tang, Z., Kang, B., Li, C., Chen, T., and Zhang, Z. (2019) GEPIA2: an enhanced web server for large-scale expression profiling and interactive analysis. Nucleic Acids Res 47, W556-W560
9. Nelson, K. J., Messier, T., Milczarek, S., Saaman, A., Beuschel, S., Gandhi, U., Heintz, N., Smalley, T. L., Lowther, W. T., and Cunniff, B. (2021) Unique Cellular and Biochemical Features of Human Mitochondrial Peroxiredoxin 3 Establish the Molecular Basis for Its Specific Reaction with Thiostrepton. Antioxidants (Basel) 10
10. Cunniff, B., Newick, K., Nelson, K. J., Wozniak, A. N., Beuschel, S., Leavitt, B., Bhave, A., Butnor, K., Koenig, A., Chouchani, E. T., James, A. M., Haynes, A. C., Lowther, W. T., Murphy, M. P., Shukla, A., and Heintz, N. H. (2015) Disabling Mitochondrial Peroxide Metabolism via Combinatorial Targeting of Peroxiredoxin 3 as an Effective Therapeutic Approach for Malignant Mesothelioma. PloS one 10, e0127310
11. Myers, C. R. (2016) Enhanced targeting of mitochondrial peroxide defense by the combined use of thiosemicarbazones and inhibitors of thioredoxin reductase. Free Radic Biol Med 91, 81-92
12. Yoshikawa, N., Kajiyama, H., Nakamura, K., Utsumi, F., Niimi, K., Mitsui, H., Sekiya, R., Suzuki, S., Shibata, K., Callen, D., and Kikkawa, F. (2016) PRIMA-1MET induces apoptosis through accumulation of intracellular reactive oxygen species irrespective of p53 status and chemo-sensitivity in epithelial ovarian cancer cells. Oncol Rep 35, 2543-2552
13. Wang, X. Y., Wang, H. J., and Li, X. Q. (2013) Peroxiredoxin III protein expression is associated with platinum resistance in epithelial ovarian cancer. Tumour Biol 34, 2275-2281
14. Li, S., Hu, X., Ye, M., and Zhu, X. (2018) The prognostic values of the peroxiredoxins family in ovarian cancer. Biosci Rep 38
15. Li, L., Shoji, W., Takano, H., Nishimura, N., Aoki, Y., Takahashi, R., Goto, S., Kaifu, T., Takai, T., and Obinata, M. (2007) Increased susceptibility of MER5 (peroxiredoxin III) knockout mice to LPS-induced oxidative stress. Biochem Biophys Res Commun 355, 715-721
16. Lee, Y. J. (2020) Knockout Mouse Models for Peroxiredoxins. Antioxidants (Basel) 9
17. Corsello, S. M., Nagari, R. T., Spangler, R. D., Rossen, J., Kocak, M., Bryan, J. G., Humeidi, R., Peck, D., Wu, X., Tang, A. A., Wang, V. M., Bender, S. A., Lemire, E., Narayan, R., Montgomery, P., Ben-David, U., Garvie, C. W., Chen, Y., Rees, M. G., Lyons, N. J., McFarland, J. M., Wong, B. T., Wang, L., Dumont, N., O'Hearn, P. J., Stefan, E., Doench, J. G., Harrington, C. N., Greulich, H., Meyerson, M., Vazquez, F., Subramanian, A., Roth, J. A., Bittker, J. A., Boehm, J. S., Mader, C. C., Tsherniak, A., and Golub, T. R. (2020) Discovering the anticancer potential of non-oncology drugs by systematic viability profiling. Nature Cancer
18. Newick, K., Cunniff, B., Preston, K., Held, P., Arbiser, J., Pass, H., Mossman, B., Shukla, A., and Heintz, N. (2012) Peroxiredoxin 3 is a redox-dependent target of thiostrepton in malignant mesothelioma cells. PloS one 7, e39404
19. Hegde, N. S., Sanders, D. A., Rodriguez, R., and Balasubramanian, S. (2011) The transcription factor FOXM1 is a cellular target of the natural product thiostrepton. Nat Chem 3, 725-731
20. Bhat, U. G., Halasi, M., and Gartel, A. L. (2009) FoxM1 is a general target for proteasome inhibitors. PloS one 4, e6593
21. Bird, K. E., Xander, C., Murcia, S., Schmalstig, A. A., Wang, X., Emanuele, M. J., Braunstein, M., and Bowers, A. A. (2020) Thiopeptides Induce Proteasome-Independent Activation of Cellular Mitophagy. ACS Chem Biol 15, 2164-2174
22. Zhang, L., Ging, N. C., Komoda, T., Hanada, T., Suzuki, T., and Watanabe, K. (2005) Antibiotic susceptibility of mammalian mitochondrial translation. FEBS Lett 579, 6423-6427
23. Harms, J. M., Wilson, D. N., Schluenzen, F., Connell, S. R., Stachelhaus, T., Zaborowska, Z., Spahn, C. M., and Fucini, P. (2008) Translational regulation via L11: molecular switches on the ribosome turned on and off by thiostrepton and micrococcin. Mol Cell 30, 26-38
24. Livnat-Levanon, N., Kevei, E., Kleifeld, O., Krutauz, D., Segref, A., Rinaldi, T., Erpapazoglou, Z., Cohen, M., Reis, N., Hoppe, T., and Glickman, M. H. (2014) Reversible 26S proteasome disassembly upon mitochondrial stress. Cell Rep 7, 1371-1380
25. Segref, A., Kevei, E., Pokrzywa, W., Schmeisser, K., Mansfeld, J., Livnat-Levanon, N., Ensenauer, R., Glickman, M. H., Ristow, M., and Hoppe, T. (2014) Pathogenesis of human mitochondrial diseases is modulated by reduced activity of the ubiquitin/proteasome system. Cell Metab 19, 642-652
26. Ayida, B. K., Simonsen, K. B., Vourloumis, D., and Hermann, T. (2005) Synthesis of dehydroalanine fragments as thiostrepton side chain mimetics. Bioorg Med Chem Lett 15, 2457-2460
27. Vaillard, V., Gonzalez, M., Perotti, J., Frau, R., and Vaillard, S. (2014) Method for the synthesis of N-alkyl-O-alkyl carbamates. RSC Advances 4, 13012-13017

### EXEMPLARY EMBODIMENT

Thiostrepton is a cyclic oligopeptide antibiotic that is also known by other names such as Bryamycin, Thiactin, alaninamide, HR4S203Y18, etc Recent studies have shown that thiostrepton also has promising anticancer activity There remains a need for thiostrepton derivatives having beneficial pharmacological properties.

In certain embodiments, the present invention provides a series of compounds having the structure of Formula (IA)

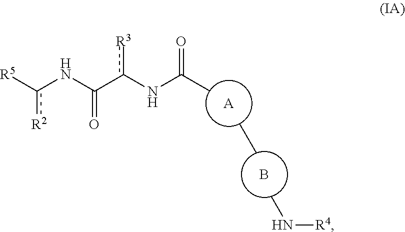

or a pharmaceutically acceptable salt thereof, wherein:
R¹ is —NH2, —NH(CH₃), —O—CH₃, or —NH—CH₂—C(O)—NH₂;
R² is —H, —CH₃, ═CH₂, or ═CH(alkyl);
R³ is —H, —CH₃, ═CH₂, or ═CH(alkyl);
R⁵ is —C(O)—R¹ or —CN;
Ring A is aryl, heteroaryl, cycloalkyl, or heterocyclyl;
Ring B is absent or present and, when present, is aryl, heteroaryl, cycloalkyl, or heterocyclyl;
R⁴ is hydrogen, a protecting group, —C(O)—CH₃, —L′, or —L—Y;
L′, when present, is a reactive linker moiety;
L, when present, is a linker moiety;
Y, when present is a mitochondrial targeting moiety;
each [symbol] is independently a single bond or a double bond; and
any hydrogen atom is optionally replaced with a deuterium.

In certain embodiments, the present invention also provides a series of compounds having the structure of Formula (IB)

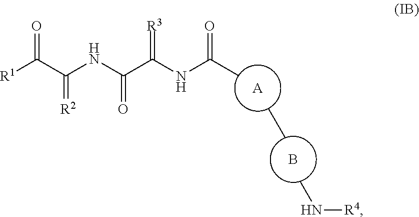

or a pharmaceutically acceptable salt thereof, wherein:
R¹ is —NH₂ or —O—CH₃;
R² is CH₂ or CH(alkyl);
R³ is CH₂ or CH(alkyl);
Ring A is heteroaryl, cycloalkyl or heterocyclyl;
Ring B is aryl, heteroaryl, cycloalkyl or heterocyclyl;
R⁴ is hydrogen, a protecting group, —C(O)—CH₃, —L′, or —L—Y;
L′, when present, is a reactive linker moiety;
L, when present, is a linker moiety;
Y, when present, is a mitochondrial targeting moiety; and
any hydrogen atom is optionally replaced with a deuterium.

Also provided herein are methods of treating cancer, comprising administering to a subject in need thereof any of the pharmaceutical compositions described herein.

### Exemplary Embodiment Pharmaceutical Compositions

The compositions and methods described herein may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound described herein and a pharmaceutically acceptable carrier. Pharmaceutically acceptable carriers are well known in the art and include, as a non-limiting example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound described herein. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound described herein. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient that can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound described herein, with the carrier and, optionally, one or more accessory ingredients. In general, the formulations are prepared by uniformly and intimately bringing into association a compound described herein with liquid carriers, or finely divided solid carriers, or both, and then, if necessary, shaping the product.

Formulations described herein suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound described herein as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound described herein to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods described herein, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors that influence the effective amount may include, but are not limited to, the severity of the patient's condition, the disorder being treated, the stability of the compound, and, if desired, another type of therapeutic agent being administered with the compound described herein. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods described herein will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds described herein may be used alone or conjointly administered with another type of therapeutic agent.

The present disclosure includes the use of pharmaceutically acceptable salts of compounds described herein in the compositions and methods described herein. In certain embodiments, contemplated salts include, but are not limited to, alkyl, dialkyl, trialkyl or tetra-alkyl ammonium salts. In certain embodiments, contemplated salts include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts include, but are not limited to, Na, Ca, K, Mg, Zn or other metal salts. In certain embodiments, contemplated salts include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d glucoheptonic acid, d gluconic acid, d glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, propionic acid, 1-pyroglutamic acid, salicylic acid, sebacic acid, stearic acid, succinic acid, sulfuric acid, 1 tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### EXEMPLARY EMBODIMENT DEFINITIONS

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification.

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., when at least 5% f drug product is detectable systemically with industry acceptable methodology, or when the two agents are simultaneously effective in the patient, which may include synergistic effects of the two agents). For example, the different therapeutic compounds can be administered either in the same formulation or in separate formulations, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic agents.

A “therapeutically effective amount” or a “therapeutically effective dose” of a compound or other agent described herein is an amount of a drug or an agent that, when administered to a subject will have the intended therapeutic effect. The full therapeutic effect does not necessarily occur by administration of one dose of such a drug or agent, and may occur only after administration of a series of doses (multiple consecutive doses). Thus, a therapeutically effective amount may be administered in one or more administrations. The precise effective amount needed for a subject will depend upon, for example, the subject's size, health and age, and the nature and extent of the condition being treated, such as cancer.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds for laboratory use, or for subsequent conversion to a pharmaceutically acceptable acid addition salt.

The term “pharmaceutically acceptable basic addition salt” as used herein means any non-toxic organic or inorganic base addition salt of any acid compounds or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in an R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure. Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce IPA or a salt thereof. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “linker” as used herein means any chemical functionality that “links,” or connects with chemical bonds, any two or more other chemical functionalities in a pharmaceutically relevant molecule. As a non-limiting example of the use of linkers in pharmaceutical settings, antibody-drug conjugations (ADCs) comprise a pharmaceutically active small-molecule, drug, or toxin connected to a large-molecule antibody by a linker.

Examples of common linker types include both cleavable and non-cleavable linkers. Cleavable linkers include chemical functionalities that can be cleaved in response to physiological stimuli such as chemical gradients, pH changes, or enzymatic activity. Non-limiting examples include acid- or base-labile functional groups, pyrophosphate diester, disulfide bonds, peptides, β-glucuronides, etc. Non-cleavable linkers comprise chemical functionalities that are generally less labile to the aforementioned physiological stimuli, and non-limiting examples include certain alkyl groups and organic polymeric functionalities.

The term “reactive linker moiety” as used herein refers to a chemical structure having a terminal moiety that can react and form a covalent bond with another moiety (such as the mitochondrial targeting moiety).

The term “mitochondrial targeting peptide,” “mitochondrial targeting sequence,” “mitochondrial targeting moiety,” as used herein are art-recognized terms referring to a chemical functionality (the peptide, sequence, or moiety), which “target,”—i.e., are readily transported to and absorbed by -mitochondrial membranes (J. Zielonka, B. Kalyanaraman, et al., 2017). As used herein, mitochondrial targeting moieties may include but are not limited to the following species: berberin cation, rhodamine cation, an indolium cation, a pyridinium cation, a tetraguanidinium cation, cyanine derivatives, a guanidinium cation, a biguanidinium cation, a triphenylphosphonium cation, a triethylammonium cation, a triphenylamine, a tetraphenylethene moiety, arylphosphonium cation, an SS peptide, a mitochondrial penetrating peptide (MPP), a mitochondrial targeting sequence (MTS) peptide, a hemigramicidin S-linked nitroxide, a Dequalinium (DQA) cation, a delocalized lipophilic cation, F16 ((E)-4-(1H-indol-3-ylvinyl)-N-methylpyridinium iodide), (L-cyclohexyl alanine-D-arginine)3, a mitochondrial-targeted nanocarrier, a DDDK peptide, glycyrrhetinic acid, α-tocopheryl succinate (a-TOS), a graphene oxide nano carrier, PEG-proapoptotic peptide (KLAKLAK)2, a Dmt-D-Arg-Phe-Lys-NH₂ peptide, pyruvaldehyde, N-Nonyl acridine orange, quinoline, styrl-azinium fluorophores, or 15d-PGJ2. Exemplary mitochondrial targeting moieties are listed in See. J Zielonka et al, Chem Rev 2017, 117, p 10043-10120; KL Horton et al, Chemistry & Biology 2008, 15, pp 375-382; G Battogtokh et al, Front Pharmacol 2018, 9:922; U.S. Pat. Nos. 9,173,952 and 9,132,198, the contents of each of which are incorporated by reference herein.

It is understood that substituents and substitution patterns on the compounds described herein can be selected by one of ordinary skilled person in the art to result in chemically stable compounds that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “C_{x-Cy}”, when used in conjunction with a chemical moiety, such as, acyl, acyloxy, alkyl, alkenyl, alkynyl, or alkoxy is meant to include groups that contain from x to y carbons in the chain. Coalkyl indicates a hydrogen where the group is in a terminal position, a bond if internal. A C₁₋₆ alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

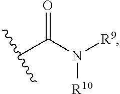

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

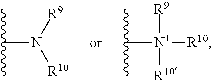

wherein R⁹, R¹⁰, and R^{10′} each independently represent a hydrogen or a hydrocarbyl group, or R9 and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” used alone or as part of a larger moiety as in “aralkyl”, “aralkoxy”, or “aryloxyalkyl”, refers to monocyclic and bicyclic ring systems having a total of five to fourteen ring members, wherein each ring atom is carbon, at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic carbocyclic rings.

The term “carbamate” is art-recognized and refers to a group

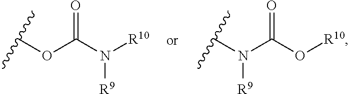

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 7L electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quatenized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridinyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl or heteroaryl rings such that the resulting bi- or multicyclic ring system as a whole is fully aromatic. Nonlimiting examples include indolyl, isoindolyl, benzothienyl, benzofuranyl, dibenzofuranyl, indazolyl, benzimidazolyl, benzothiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, and phenoxazinyl. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

As used herein, the terms “heterocycle”, “heterocyclyl”, “heterocyclic radical”, and “heterocyclic ring” are used interchangeably and refer to a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO3H, or a pharmaceutically acceptable salt thereof.

The term “sulfonamide” is art-recognized and refers to the group represented by the general formulae

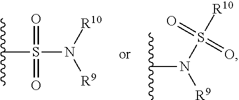

wherein R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “protecting group” is an art-recognized term referring to chemical functionalities that can modify (usually covalently) an existing labile functionality on a target molecule. This modification “protects” the labile functionality during subsequent reaction steps, and the protecting group can be removed as needed, termed “deprotection.” As a non-limiting example, the t-butyloxycarbonyl (Boc or boc) group is commonly used to covalently modify and “protect” terminal amine groups in synthetic chemistry.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfuydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

Suitable monovalent substituents on a substitutable carbon atom of an “optionally substituted” group are independently halogen; —(CH₂)₀₋₄R^{∘}; —(CH₂)₀₋₄R^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₄Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})2; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(QR^{∘})R^{∘}; —C(Q)C(O)R^{∘}, —C(Q)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}2; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{∘}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₄Ph, or a 5-7-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂═NNHC(O)R*, ═NNHC(O)OR^{●}, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃—O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}2, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)QR^{†}, —C(O)C(O)R^{†}, —C(O)CH₂C(O)R^{†}, —S(O)₂R^{†}, —S(O)₂NR₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or a substituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group and the substituted 5-6-membered saturated, partially unsaturated, or aryl ring having 0-4 heteroatoms independently selected from nitrogen, oxygen, or sulfur of RT independently halogen, —R^{●}, -(haloR^{●}), —OH, —OR^{●}, —O(haloR^{●}), —CN, —C(O)OH, —C(O)OR′, —NH₂, —NHR^{●}, —NR^{∘}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

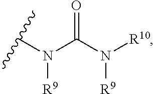

wherein R⁹ and R¹⁰ independently represent hydrogen or a hydrocarbyl.

The term “tautomer” refers to each of two or more isomers of a compound that exist together in equilibrium, and are interchanged by migration of an atom or group within the molecule, such as a hydrogen atom. Exemplary tautomers of the present disclosure include,

but are not limited to

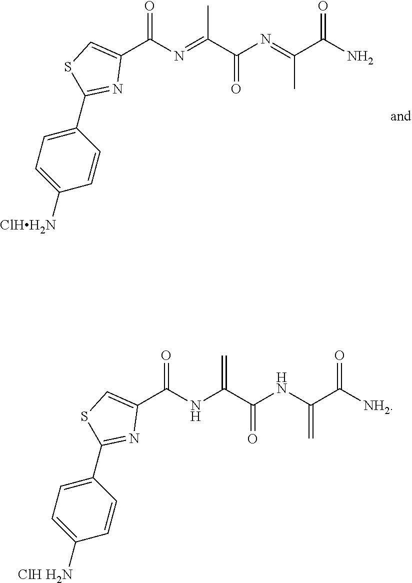

Depiction in this disclosure of one tautomer within a genus or compound species is intended to encompass the compound as drawn and all of its tautomer's. Specifically, for the example above, either of those structures also discloses

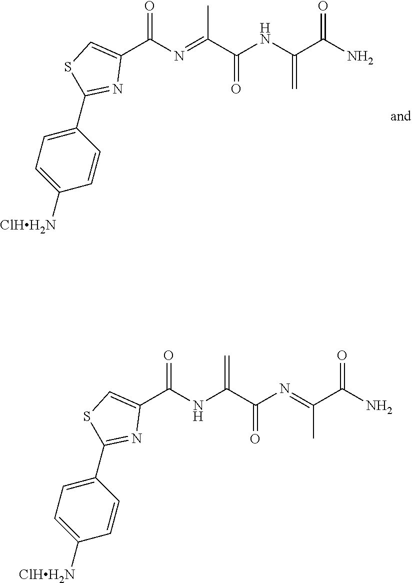

for a series of 2n distinct species where n is the number of tautomeric sites on the molecule.

For the purposes of this disclosure, any of the embodiments described herein applies to any of the generic structural formulas described herein, if properly dependent.

In certain embodiments, the present invention provides a series of compounds compound having the structure of Formula (IA)

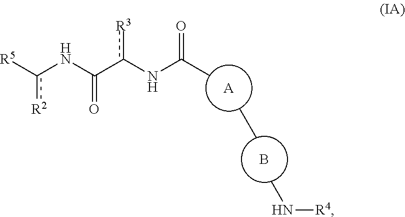

or a pharmaceutically acceptable salt thereof, wherein:
R¹ is —NH₂, —NH(CH₃), —O—CH₃, or —NH—CH₂—C(O)—NH₂;
R² is —H, —CH₃, ═CH₂, or ═CH(alkyl);
R³ is —H, —CH₃, ═CH₂, or ═CH(alkyl);
R⁵ is —C(O)—R¹ or —CN;
Ring A is aryl, heteroaryl, cycloalkyl, or heterocyclyl;
Ring B is absent or present and, when present, is aryl, heteroaryl, cycloalkyl, or heterocyclyl;
R⁴ is hydrogen, a protecting group, —C(O)—CH₃, —L′, or —L—Y;
L′, when present, is a reactive linker moiety;
L, when present, is a linker moiety;
Y, when present, is a mitochondrial targeting moiety;
each [symbol] is independently a single bond or a double bond; and any hydrogen atom is optionally replaced with a deuterium.

In certain embodiments, the present invention provides a series of compounds having the structure of Formula (2-1)

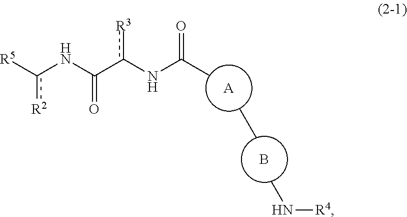

or a pharmaceutically acceptable salt thereof, wherein:
R¹ is —NH₂, —NH(CH₃), —O—CH₃, or —NH—CH₂—C(O)—NH₂;
R² is —H, —CH₃, ═CH₂, or ═CH(alkyl);
R³ is —H, —CH₃, ═CH₂, or ═CH(alkyl);
R⁵ is —C(O)—R¹ or —CN;
Ring A is aryl, heteroaryl, cycloalkyl, or heterocyclyl;
Ring B is absent or present and, when present, is aryl, heteroaryl, cycloalkyl, or heterocyclyl;
R⁴ is hydrogen, a protecting group, —C(O)—CH₃, —L′, or —L—Y;
L′, when present, is a reactive linker moiety comprising an alkyne group;
L, when present, is a linker moiety;
Y, when present, is a mitochondrial targeting moiety;
each [symbol] is independently a single bond or a double bond;
any hydrogen atom is optionally replaced with a deuterium;
provided that if Ring A is selected from unsubstituted thiazolyl, oxazolyl, cyclopentyl,
   and bicyclo[2.1.1] hexane and Ring B—NIR⁴ is

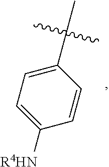

then
R⁴ IS—L′—Y or L; OR R² and R³ are not both ═CH₂ or both ═CH(alkyl).

In certain embodiments, L is

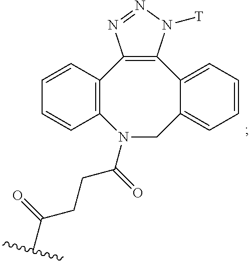

T is selected from alkyl and (—CH₂CH₂—O—)ᵥ—; v is an integer selected from 3-9; and T is bonded to Y.

In certain embodiments, the present invention also provides a series of compounds having the structure of Formula (2-2)

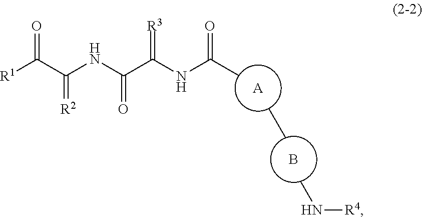

or a pharmaceutically acceptable salt thereof, wherein:
R¹ is —NH₂, or —O—CH₃;
R² is ═CH₂, or ═CH(alkyl);
R³ is ═CH₂, or ═CH(alkyl);
Ring A is heteroaryl, cycloalkyl or heterocyclyl;
Ring B is aryl, heteroaryl, cycloalkyl or heterocyclyl;
R⁴ is hydrogen, a protecting group, —C(O)—CH₃, —L′, or —L—Y;
L′, when present, is a reactive linker moiety comprising an alkyne group;
L, when present, is a linker moiety;
Y, when present, is a mitochondrial targeting moiety; and
any hydrogen atom is optionally replaced with a deuterium;
provided that if Ring A is selected from unsubstituted thiazolyl, oxazolyl, cyclopentyl, and bicyclo[2.1. 1] hexane and Ring B—NHR⁴ is

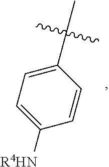

then R⁴ is —L′—Y or L, or R² and R³ are not both ═CH₂ or both ═CH(alkyl).

In certain embodiments, L is

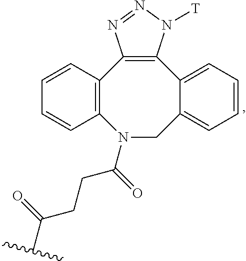

T is selected from alkyl and (—CH₂CH₂-0-)ᵥ—; v is an integer selected from 3-9; and T is bonded to Y.

In certain embodiments, the present invention also provides a series of compounds having the structure of Formula (2-3)

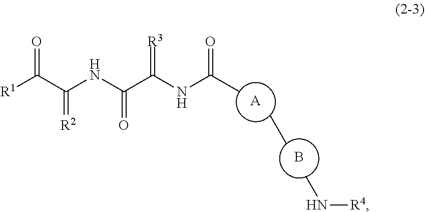

or a pharmaceutically acceptable salt thereof, wherein
R¹ is —NH₂, or —O—CH₃;
R² is ═CH₂, or ═CH(alkyl);
R³ is ═CH₂, or ═CH(alkyl);
Ring A is heteroaryl, cycloalkyl or heterocyclyl;
Ring B is aryl, heteroaryl, cycloalkyl or heterocyclyl;
R⁴ is hydrogen, a protecting group, or —C(O)—CH₃, and
any hydrogen atom is optionally replaced with a deuterium;
provided that if Ring A is selected from unsubstituted thiazolyl, oxazolyl, cyclopentyl, and bicyclo[2 1.1] hexane and Ring B—NR⁴ is

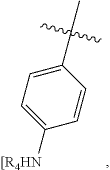

then R² and R³ are not both ═CH₂ or both ═CH(alkyl).

In certain embodiments, the present invention provides a series of compounds having the structure of Formula (2-4)

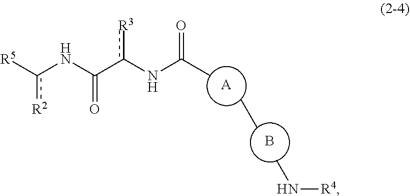

or a pharmaceutically acceptable salt thereof, wherein;
R¹ is —NH₂, —NH(CH₃), —O—CH₃, or —NH—CH₂—C(O)—NH₂;
R² is —H, —CH₃, ═CH₂, or ═CH(alkyl);
R³ is —H, —CH₃, ═CH₂, or ═CH(alkyl);
R⁵ is —C(O)—R¹ or —CN;
Ring A is selected from

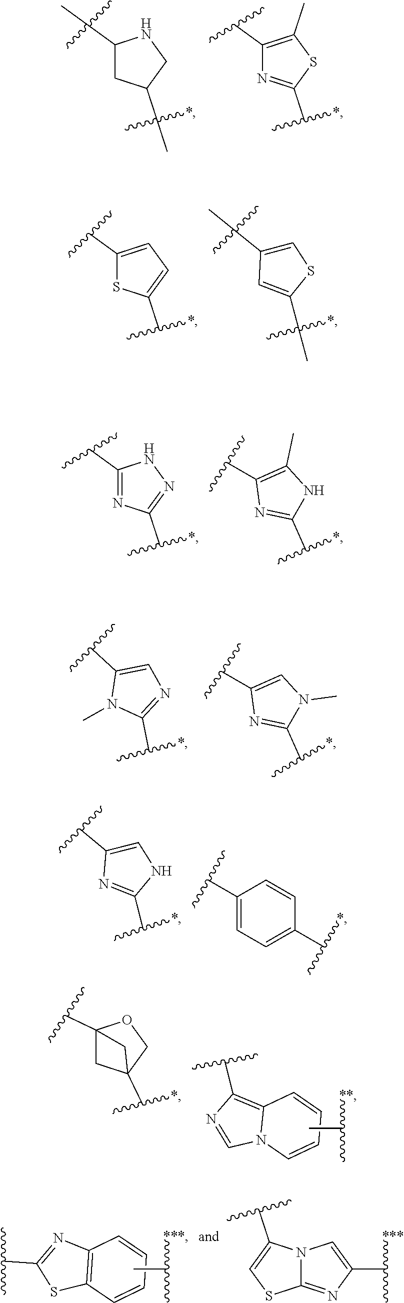

wherein * denotes a bond to Ring B and ** and *** denote —NH—R⁴;
Ring B is absent or present and, when present, is aryl, heteroaryl, cycloalkyl, or heterocyclyl;
R⁴ is hydrogen, a protecting group, —C(O)—CH₃, —L′, or —L—Y;
L′, when present, is a reactive linker moiety comprising an alkyne group;
L, when present, is a linker moiety;
Y, when present is a mitochondrial targeting moiety;
each [symbol] is independently a single bond or a double bond; and
any hydrogen atom is optionally replaced with a deuterium.

In certain embodiments, L is

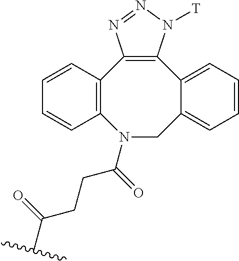

T is selected from alkyl and (—CH₂CH₂-0-)ᵥ—; v is an integer selected from 3-9; and T is bonded to Y.

In certain embodiments, the present invention provides a series of compounds having the structure of Formula (2-5)

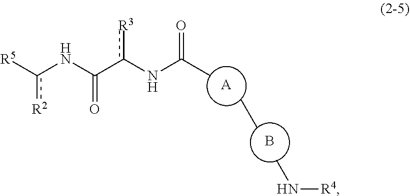

or a pharmaceutically acceptable salt thereof, wherein:
R¹ is —NH₂, —NH(CH₃), —O—CH₃, or —NH—CH₂—C(O)—NH₂;
R² is —H, —CH₃, ═CH₂, or ═CH(alkyl);
R³ is —H, —CH₃, ═CH₂, or ═CH(alkyl);
R⁵ is —C(O)—R¹ or —CN;
Ring A is aryl, heteroaryl, cycloalkyl, or heterocyclyl;
Ring, B—NHR⁴ is selected from

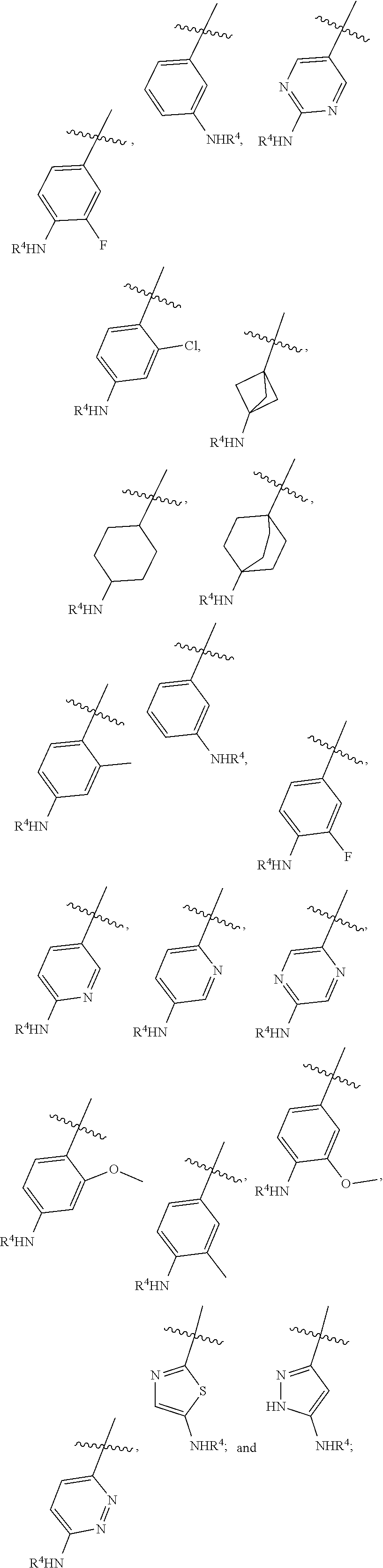

R⁴ is hydrogen, a protecting group, —C(O)—CH₃, —L′, or —L—Y;
L′, when present, is a reactive linker moiety comprising an alkyne group;
L, when present, is a linker moiety;
Y, when present, is a mitochondrial targeting moiety;
each [symbol] is independently a single bond or a double bond; and
any hydrogen atom is optionally replaced with a deuterium

In certain embodiments, L is

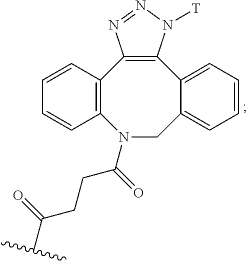

T is selected from alkyl and (—CH₂CH₂-0-)ᵥ—; v is an integer selected from 3-9; and T is bonded to Y.

In certain embodiments, the present invention provides a series of compounds having the structure of Formula (2-6):

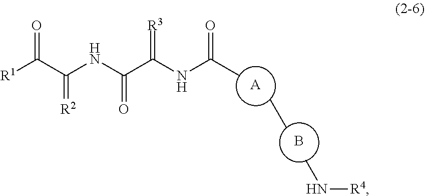

or a pharmaceutically acceptable salt thereof, wherein
R¹ is —NH₂, or —O—CH₃;
R² is ═CH₂, or ═CH(alkyl);
R³ is ═CH₂, or ═CH(alkyl);
ring A is thiazolyl substituted with alkyl or thiophenyl;
Ring B—NHR⁴ is

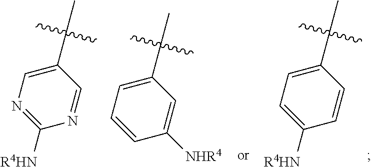

R⁴ is a protecting group or —L′,
L′, when present, is a reactive linker moiety comprising an alkyne group;
any hydrogen atom is optionally replaced with a deuterium.

In certain embodiments, R⁵ is —C(O)—R¹; and R¹ is —OCH₃. In other embodiments, R⁵ is —C(O)—R¹, and R¹.—NH₂

In certain embodiments, when Ring A is polycyclic, then Ring B is absent.

In certain embodiments, Ring A is a 5-membered ring or a 5-membered ring fused to a second ring.

In certain embodiments, Ring A is a 5-membered heteroaryl.

In some embodiments, Ring A is a bicyclic heteroaryl.

In certain embodiments, Ring A is thiazolyl, thiophenyl, oxazolyl, or imidazolyl.

In other embodiments, Ring A is thiazolyl, thiophenyl, or oxazolyl.

In some embodiments, Ring A is pyrrolidinyl.

In other embodiments, Ring A is thiazolyl.

In certain embodiments, Ring A is

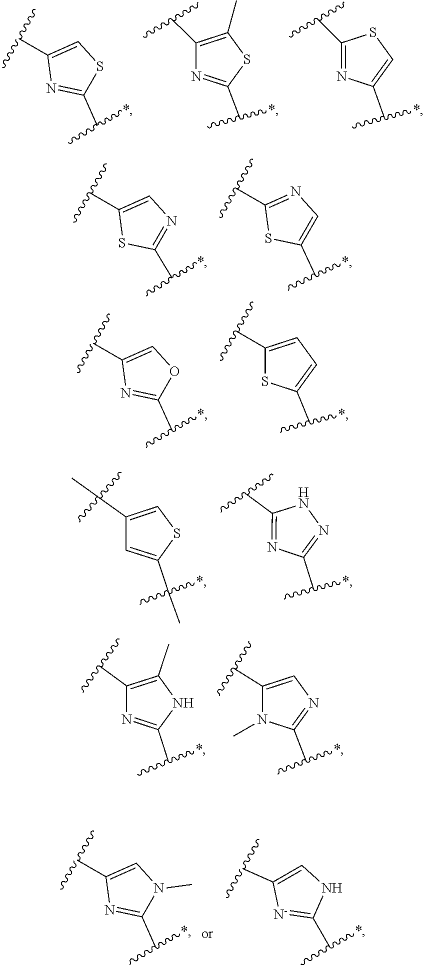

wherein * denotes a bond to Ring B.

In certain embodiments, Ring A is

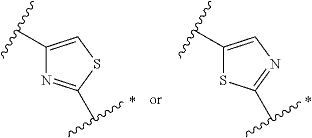

wherein * denotes a bond to Ring B.

In certain embodiments, Ring A is

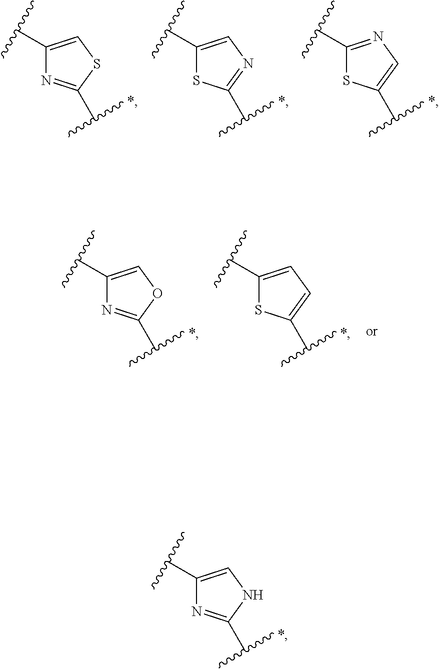

wherein * denotes a bond to Ring B.

In certain embodiments, Ring A is

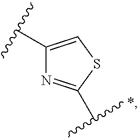 [SMILES: *c1csc(*)n1]

wherein * denotes a bond to Ring B.

In certain embodiments, Ring A is a 5-membered cycloalkyl or heterocyclyl.

In certain embodiments, Ring A is cyclopentyl or tetrahydrofuranyl.

In certain embodiments, Ring A is

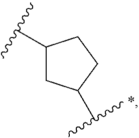 [SMILES: *C1CCC(*)C1]

wherein * denotes a bond to Ring B.

In certain embodiments, Ring A is a bridged bicyclic cycloalkyl or heterocyclyl.

In certain embodiments, Ring A is bicyclo[2.1.1]hexyl or oxabicyclo[2.1.1]hexyl.

In certain embodiments, Ring A is

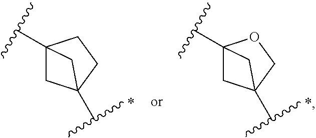

wherein * denotes a bond to Ring B.

In certain embodiments, wherein Ring A is phenyl.

In certain embodiments, Ring A is

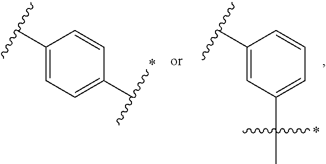 [SMILES: *c1ccc(*)cc1 ; *c1cccc(*)c1]

or wherein * denotes a bond to Ring B.

In certain embodiments, Ring A is a polycyclic aryl, heteroaryl, cycloalkyl, or heterocyclyl.

In certain embodiments, Ring A is a bicyclic heteroaryl.

In certain embodiments, Ring A is

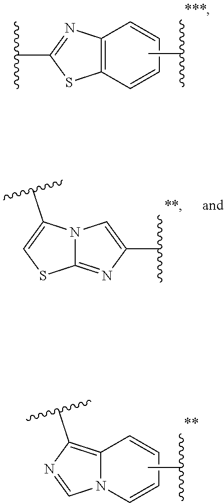

wherein *** and ** denote Ring B or —NH—R⁴.

In certain embodiments, Ring B is a 6-membered ring.

In certain embodiments, B is phenyl.

In certain embodiments, Ring B is unsubstituted phenyl. In other embodiments, Ring B is a halogen-substituted phenyl.

In certain embodiments, Ring B is

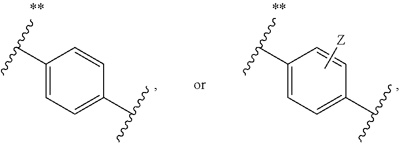

wherein Z is halo and ** denotes a bond to Ring A. In some embodiments, Z is selected from fluoro or chloro.

In certain embodiments, Ring B is

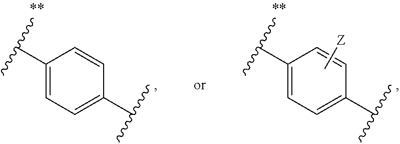

wherein Z is alkoxy, such as methoxy, or alkyl, such as methyl.

In other embodiments, Ring B is

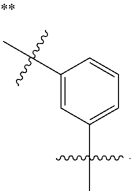 [SMILES: *c1cccc(*)c1]

In certain embodiments, Ring B is a 6-membered heteroaryl.

In certain embodiments, Ring B is pyridinyl, pyrimidinyl pyrazinyl, or pyridazinyl.

In certain embodiments, Ring B is pyridinyl, pyrimidinyl or pyrazinyl.

In certain embodiments, Ring B is

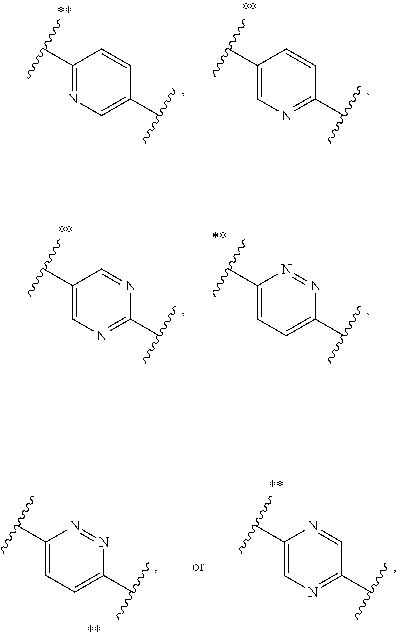 [SMILES: *c1ccc(*)nc1 ; *c1ccc(*)nc1 ; *c1cnc(*)nc1 ; *c1ccc(*)nn1 ; *c1ccc(*)nn1 ; *c1cnc(*)cn1]

wherein ** denotes a bond to Ring A.

In certain embodiments, Ring B is a bridged bicyclic cycloalkyl.

In certain embodiments, Ring A is bicyclo[2.2.2]octanyl or bicyclo [1.1.1]pentanyl.

In certain embodiments, Ring B is

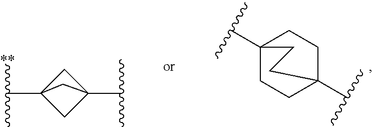

wherein Z is halo and ** denotes a bond to Ring A.

In certain embodiment, Ring B is selected from

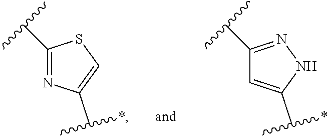

where * denotes a bond to —NHR⁴.

In certain embodiments, R⁵ is —CN.

In certain embodiments, the compound of Formula (IA) has the structure of Formula (IA-1)

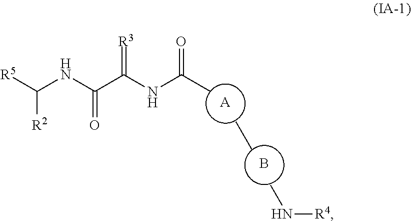

or a pharmaceutically acceptable salt thereof, wherein;
R² is —H or —CH₃; and
R³ is ═CH₂ or ═CH(alkyl); and
each of Ring A, Ring B, R¹, R⁴ and R⁵ are any of the embodiments set forth above for Formula (IA).

In certain embodiments of Formula (IA-1), R² is —H and R³ is ═CH₂, or R2 is —CH₃ and R³ is ═CH₂.

In certain embodiments, the compound of Formula (IA) has the structure of Formula (IA-2):

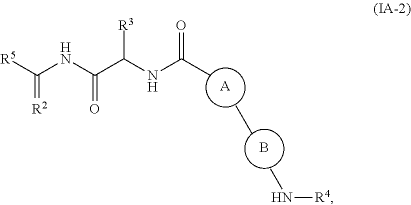

or a pharmaceutically acceptable salt thereof,

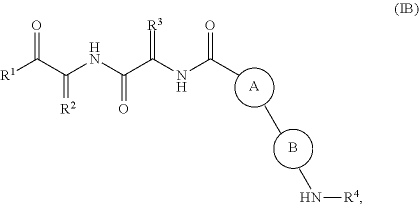

or a pharmaceutically acceptable salt
wherein;
R² is ═CH₂ or ═CH(alkyl); and
R³ is —H or —CH₃; and
each of Ring A, Ring B, R¹, R⁴ and R⁵ are any of the embodiments set forth above for Formula (IA).

In certain embodiments of Formula (IA-2), R² is ═CH₂ and R³ is —H, or R² is ═CH₂ and R³ is —CH₃.

In certain embodiments, the compound of Formula (IA) has the structure of Formula (IA-3)

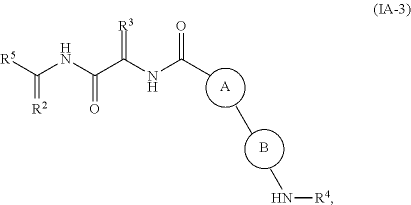

or a pharmaceutically acceptable salt thereof, wherein;
R² is ═CH₂ or ═CH(alkyl); and
R³ is ═CH₂ or ═CH(alkyl); and
each of Ring A, Ring B, R¹, R⁴ and R⁵ are any of the embodiments set forth above for Formula (IA).

In certain embodiments of Formula (IA-3), R² is ═CH₂ and R³ is ═CH₂, R² is ═CH₂ and R³ is ═CH(CH₃), or R² is ═CH(CH₃) and R³ is ═CH₂.

In certain embodiments, the compound of Formula (IA) has the structure of Formula (IB)

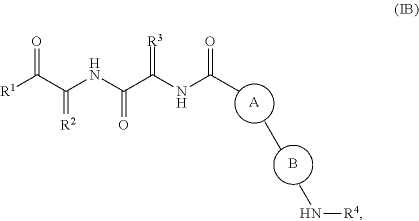

or a pharmaceutically acceptable salt thereof, wherein:
R¹ is —NH₂ or —O—CH₃;
R² is CH₂ or CH(alkyl);
R³ is CH₂ or CH(alkyl);
Ring A is heteroaryl, cycloalkyl or heterocyclyl;
Ring B is aryl, heteroaryl, cycloalkyl or heterocyclyl;
R⁴ is hydrogen, a protecting group, —C(O)—CH₃, —L′, or —L—Y;
L′, when present, is a reactive linker moiety;
L, when present, is a linker moiety;
Y, when present, is a mitochondrial targeting moiety; and
any hydrogen atom is optionally replaced with a deuterium.

In certain embodiments of Formula (IB), Ring A is a 5-membered ring or a 5-membered ring fused or bridged to a second ring.

In certain embodiments, Ring A is thiazolyl, thiophenyl, oxazolyl, cyclopentyl, or bicyclo[2.1.1]hexyl.

In certain embodiments of Formula (IB), Ring A is thiazolyl.

In certain embodiments of Formula (IB), Ring A is: N or.

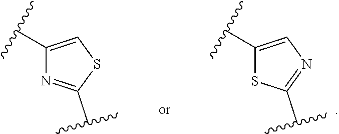 [SMILES: *c1csc(*)n1 ; *c1cnc(*)s1]

In certain embodiments of Formula (IB), Ring A is

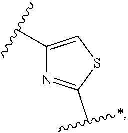

wherein * denotes a bond to Ring B.

In certain embodiments of Formula (IB), Ring B is a six-membered ring.

In certain embodiments, Ring B is phenyl or pyridyl.

In certain embodiments, Ring B is unsubstituted phenyl.

In certain embodiments of Formula (IB), Ring B is a halogen-substituted phenyl.

In certain embodiments of Formula (IB), R² and R³ are different.

In certain embodiments of Formula (IB), R² and R³ are the same.

In certain embodiments, R² is CH(Me) or —CH₂.

In certain embodiments of Formula (IB), R³ is CH(Me) or —CH₂.

In certain embodiments of Formula (IB), R² and R³ are each CH₂.

In certain embodiments of Formula (IB), R⁴ is hydrogen, a protecting group, or —C(O)—CH₃.

In certain embodiments, the compound is selected from:

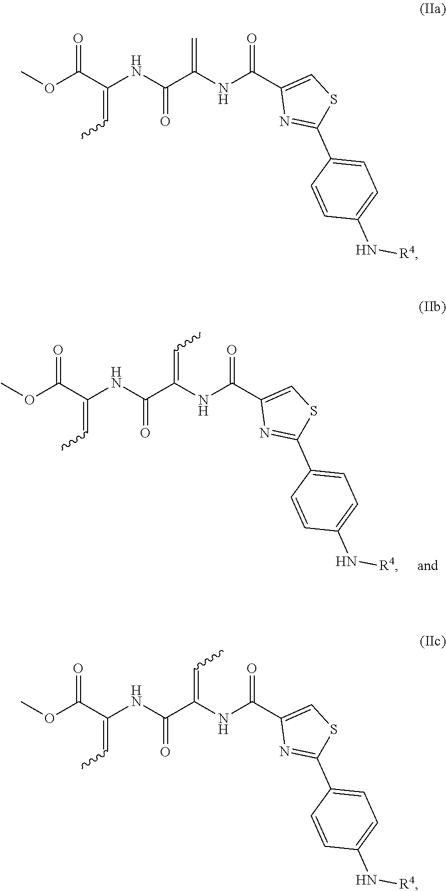

wherein R⁴ is hydrogen, a protecting group, or —C(O)—CH₃; and
wherein a bond drawn as “ [symbol] ” denotes either possible stereochemistry of the attached alkene, E or Z.

In certain embodiments, the compound is selected from

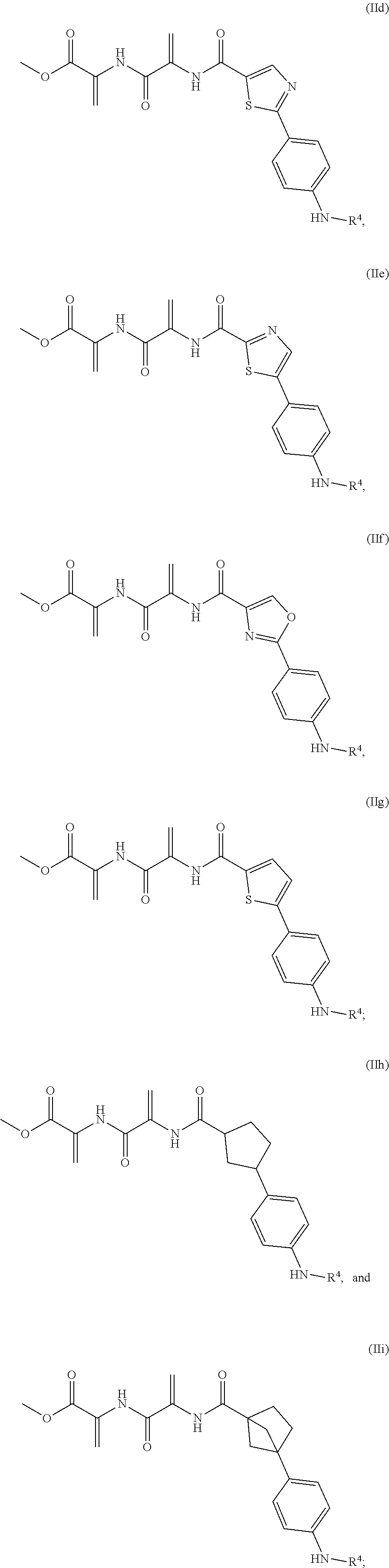

wherein R⁴ is hydrogen, a protecting group, or —C(O)—CH₃.

In certain embodiments, the compound is selected from

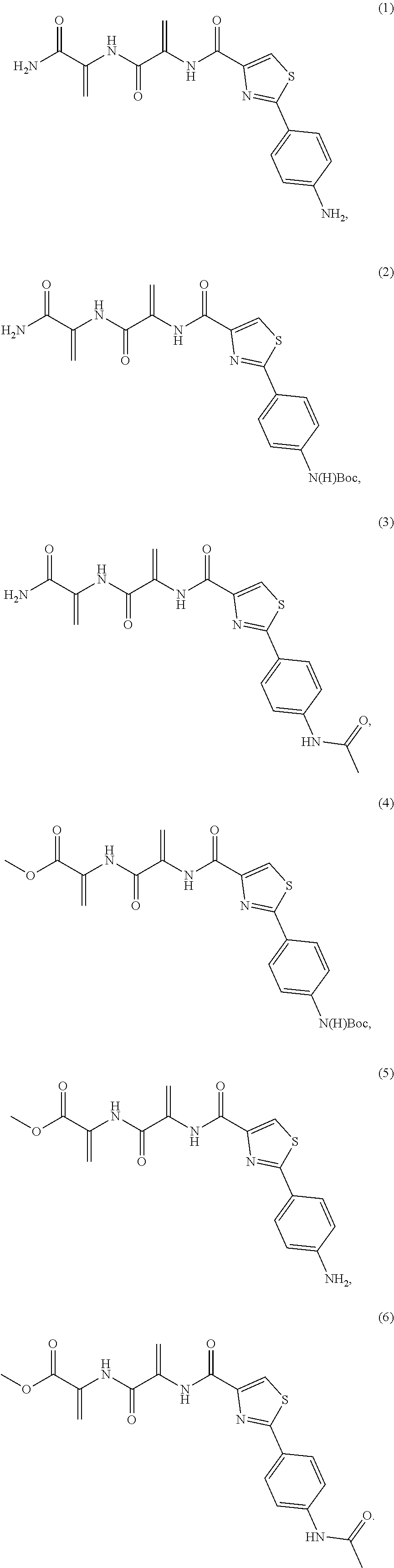

In certain embodiments, the compound is selected from

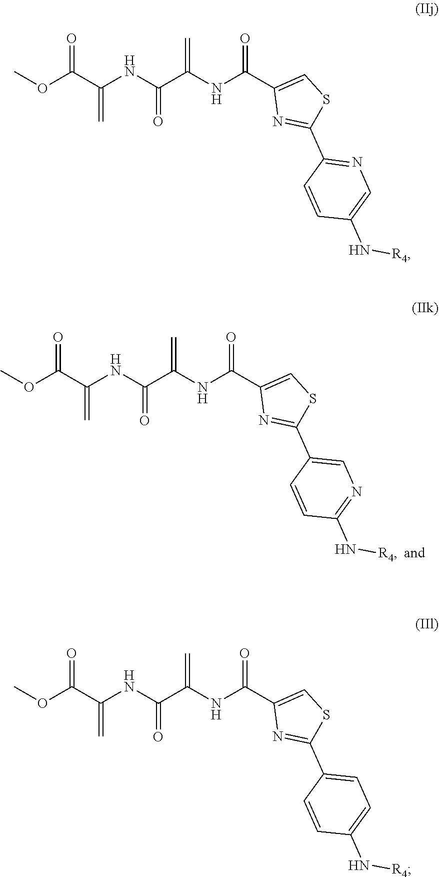

wherein R⁴ is hydrogen, a protecting group, or —C(O)—CH₃; and
wherein Z is selected from fluorine, chlorine, bromine, and iodine.

In certain embodiments, the compound is selected from:

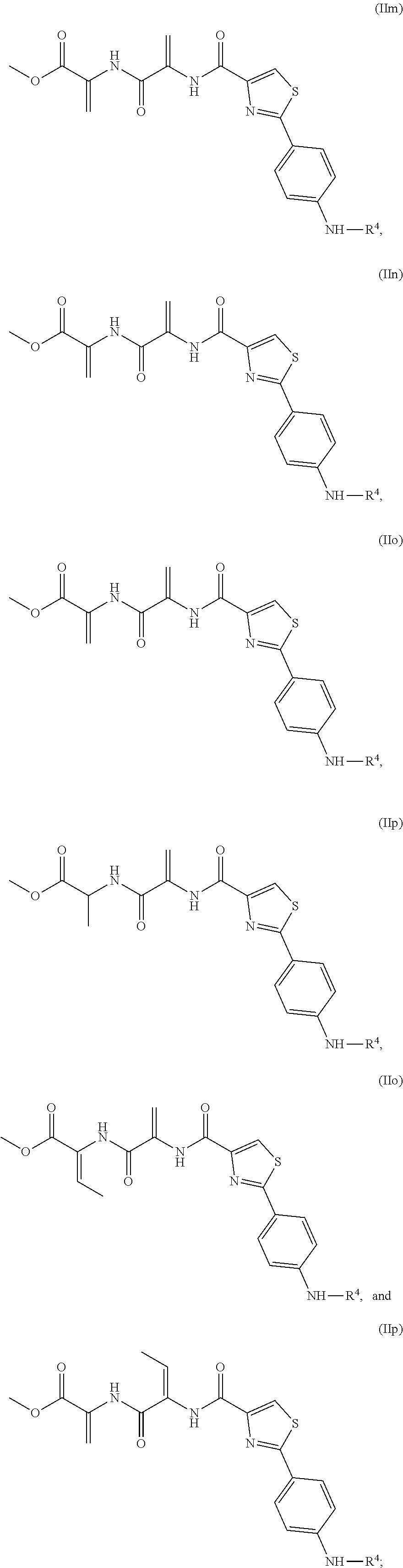

wherein R⁴ is hydrogen, a protecting group, or —C(O)—CH₃.

In certain embodiments, the compound is selected from:
wherein R⁴ is hydrogen, a protecting group, or —C(O)—CH₃.

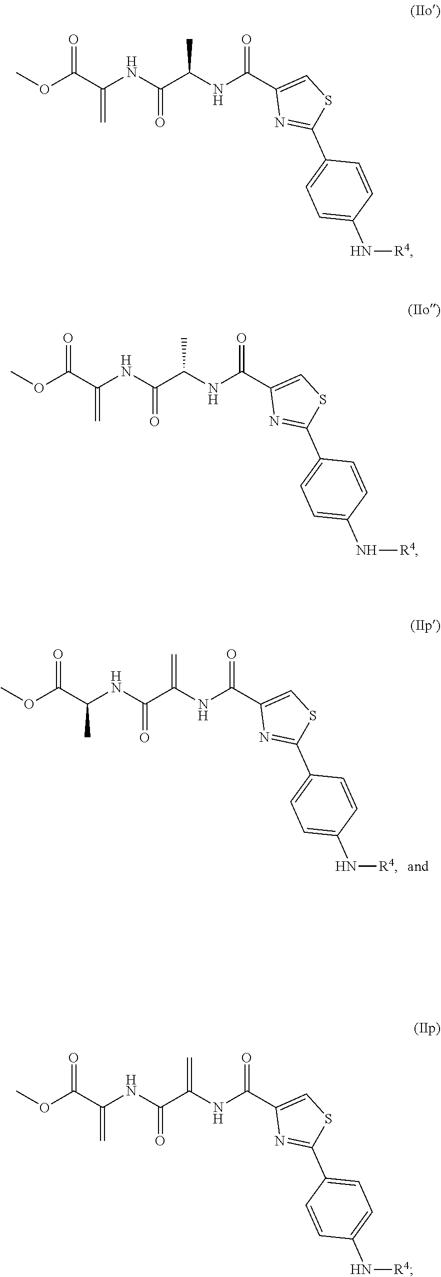

In certain embodiments, the protecting group is floe.

In certain embodiments, R⁴ is —L′.

In certain embodiments, L′ is —C(O)—X—C(O)OH or —C(O)—X—C(O)NH₁₂; X is —(CH₂)ₙ—, and n is 2, 3, 4 or 5.

In certain embodiments, the compound is selected from

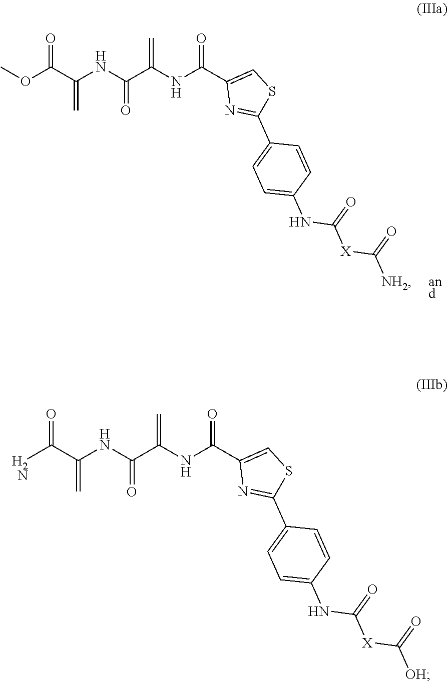

wherein X is —(CH²)ₙ—, and n is 2, 3, 4 or 5.

In certain embodiments, the compound is selected from

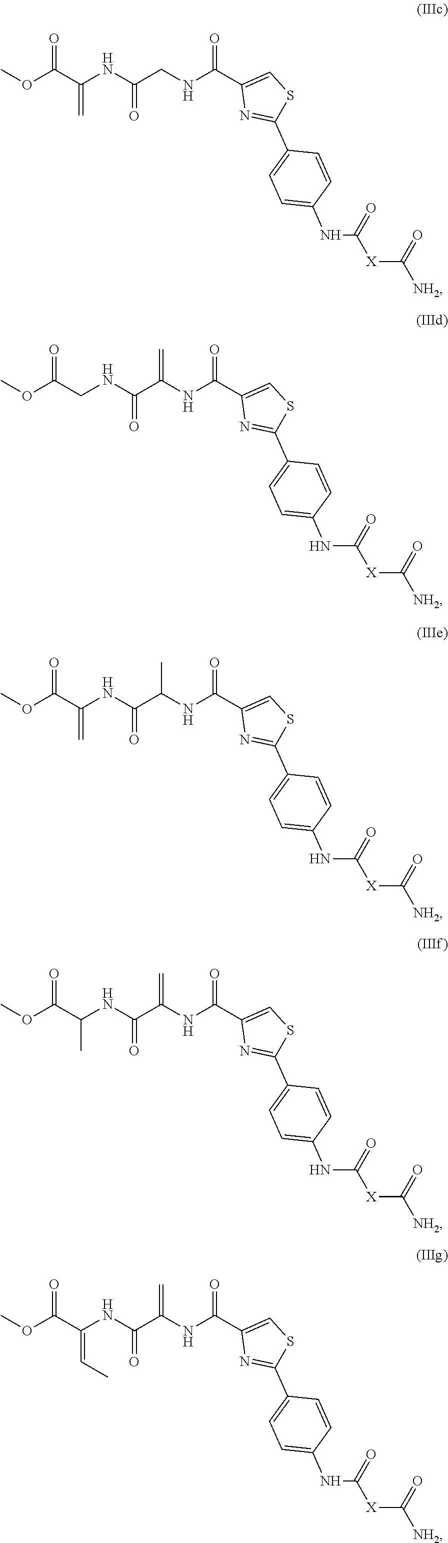

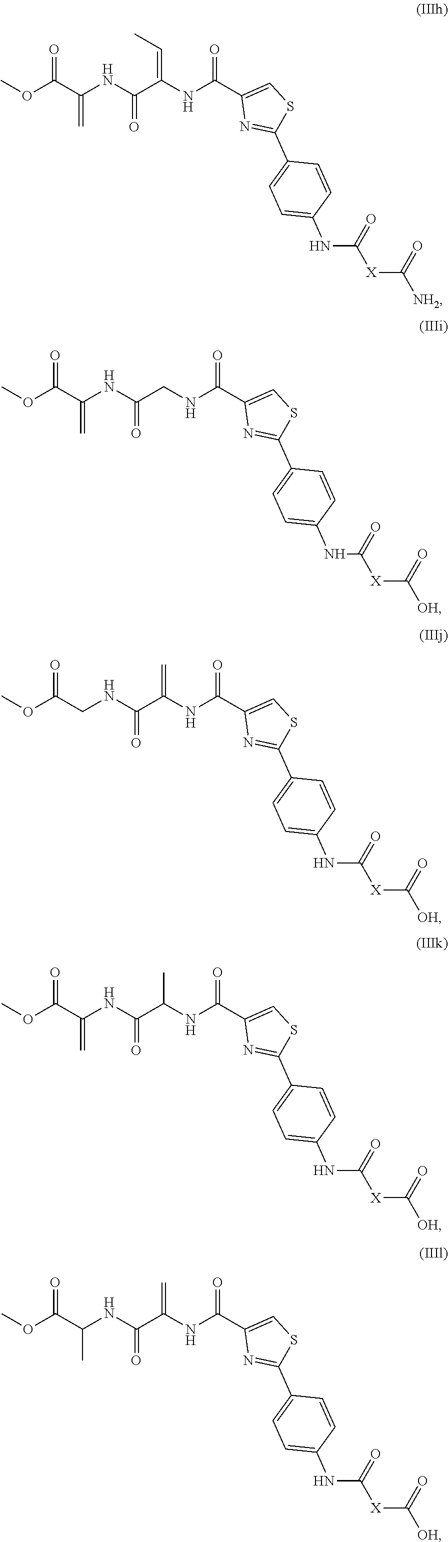

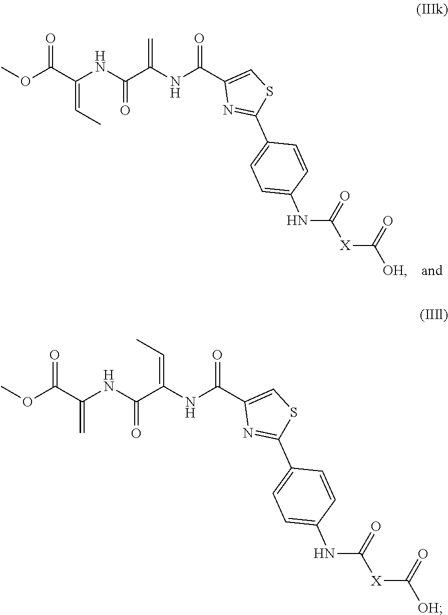

wherein X is —(CH₂)ₙ—; and
n is 2, 3,4 or 5.

In certain embodiments, R⁴ is —L—Y.

In certain embodiments, L is a cleavable linker.

In certain embodiments, L is a non-cleavable linker.

In certain embodiments, L has a chain length of about 2 to about 30 atoms.

In certain embodiments, L has a chain length of about 5 to about 20 atoms.

In certain embodiments, L is —C(O)—X—C(O)—; X is —(CH₂)ₙ—; and n is 2, 3, 4 or 5.

In certain embodiments, L is —C(O)—X—C(O)—; X is —(CH₂CH₂—O—)ₘ—(CH₂CH₂)—; and m is 2, 3, 4, 5, or 6.

In certain embodiments, R⁴ is L′ wherein the alkyne group is

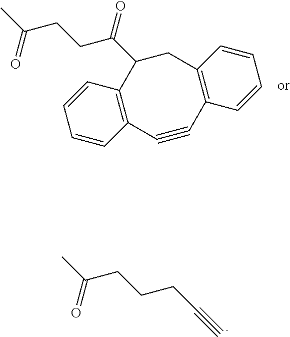

In certain embodiments, the compound is selected from

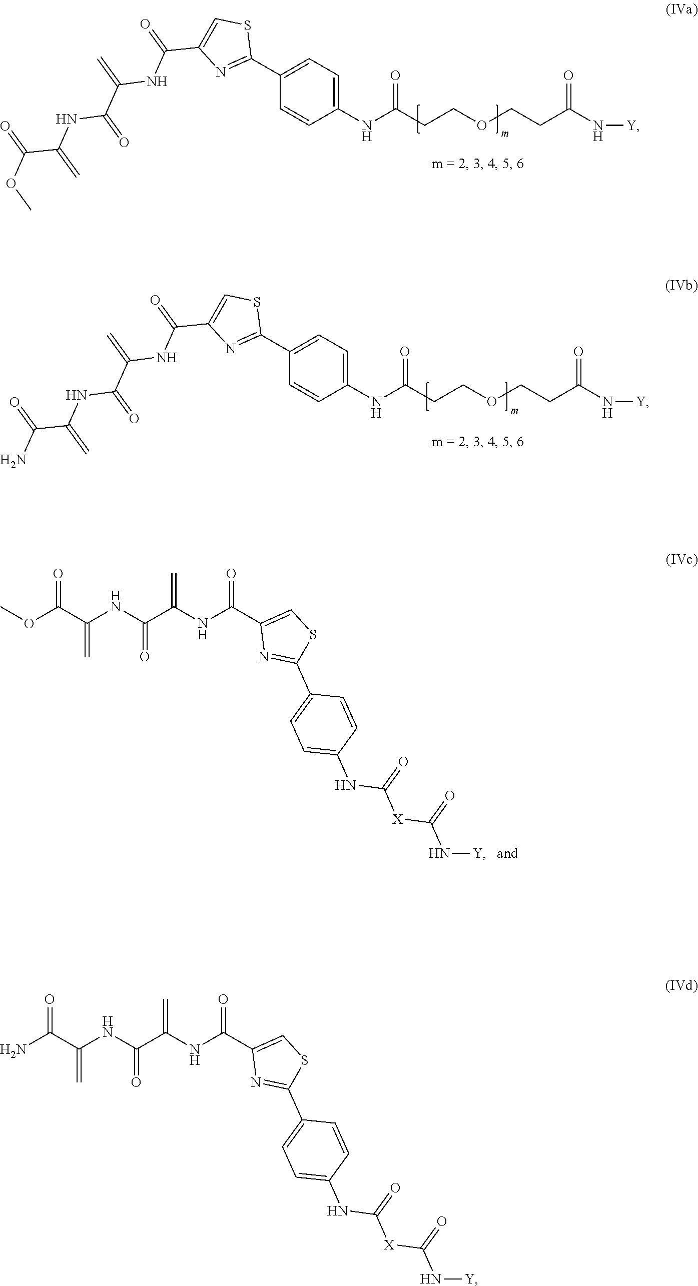

wherein X is —(CH₂)ₙ—; and n is 2, 3, 4 or 5.

In certain embodiments, the compound is selected from

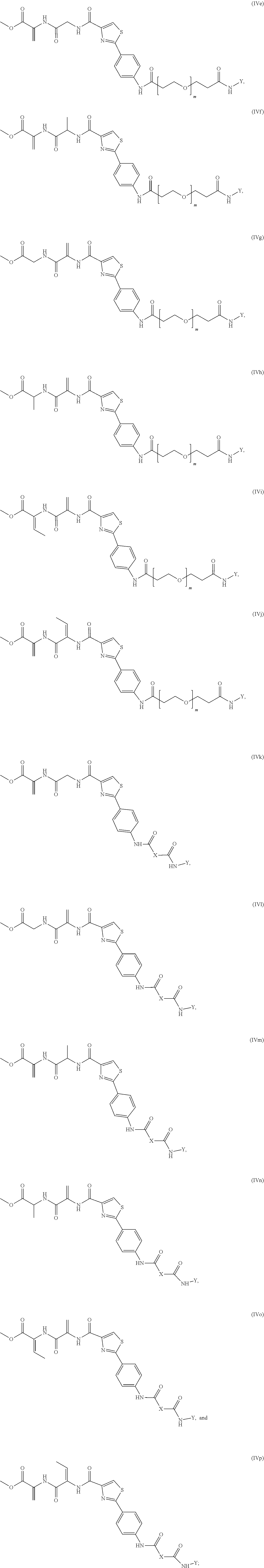

wherein X is —(CH₂)ₙ—;
m is 2, 3, 4, 5, or 6; and
n is 2, 3,4 or 5.

In certain embodiments, L′ comprises an alkynyl or azido.

In certain embodiments, wherein R⁴ is —C(O)—X′—C═CH or —C(O)—X′—N₃; X′ is —(CH₂)ₙ—; and n is 2, 3, 4 or 5.

In certain embodiments, L comprises a heteroaryl. In other embodiments, L comprises a triazolyl.

In certain embodiments, R⁴ is or

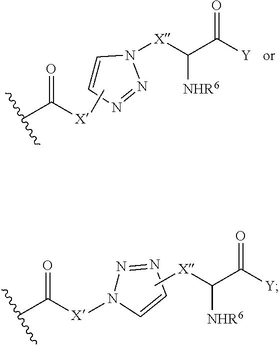

R⁶ is —H or —C(O)CH₃;
X′ is —(CH₂)ₙ—;
X” is —(CH₂)ₒ—;
n is 2, 3, 4 or 5; and
o is 2, 3, 4 or 5

In certain embodiment, R⁴ is

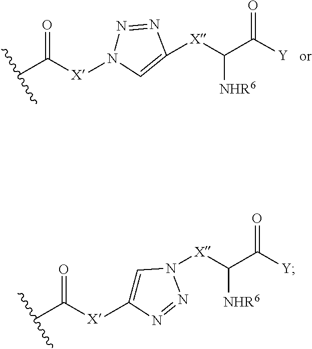

R⁶ is —H or —C(O)CH₃;
X′ is —(CH₂)ₙ—;
X″ is —(CH₂)ₒ—;
n is 2, 3, 4 or 5; and
o is 2, 3,4 or 5.

In certain embodiments, R⁴ is

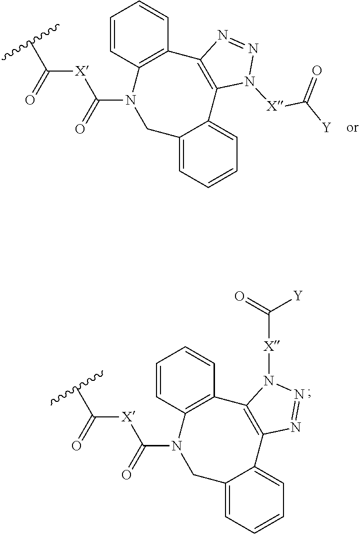

X′ is —(CH₂)ₙ—;
X″ is —(CH₂)ₒ—;
n is 2, 3, 4 or 5; and
o is 2, 3,4 or 5.

In certain embodiments, Y is a berberin cation, rhodamine cation, an indolium cation, a pyridinium cation, a tetraguanidinium cation, cyanine derivatives, a guanidinium cation, a biguanidinium cation, a triphenylphosphonium cation, a triethylammonium cation, a triphenylamine, a tetraphenylethene moiety, arylphosphonium cation, an SS peptide, a mitochondrial penetrating peptide (MPP), a mitochondrial targeting sequence (MTS) peptide, a hemigramicidin S-linked nitroxide, a Dequalinium (DQA) cation, a delocalized lipophilic cation, F16 ((E)-4-(1H-indol-3-ylvinyl)-N-methylpyridinium iodide), (Lcyclohexyl alanine-D-arginine)₃, a mitochondrial-targeted nanocarrier, a DDDK peptide, glycyrrhetinic acid, α-tocopheryl succinate (α-TOS), a graphene oxide nano carrier, PEG-proapoptotic peptide (KLAKLAK)₂, a Dmt-D-Arg-Phe-Lys-NH₂ peptide, pyruvaldehyde, N-Nonyl acridine orange, quinoline, styryl fluorophores, or 15d-PGJ2.

In certain embodiments, Y is a mitochondrial penetrating peptide.

In certain embodiments, Y has the structural formula (V):

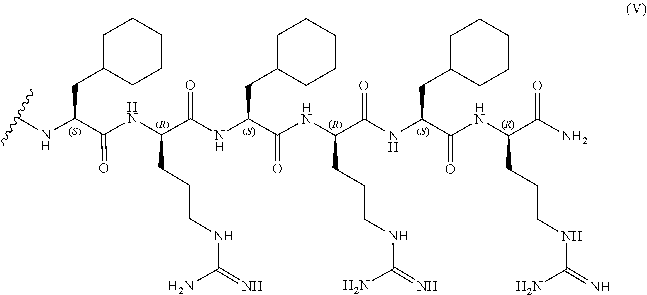

In certain embodiments, the compound is selected from

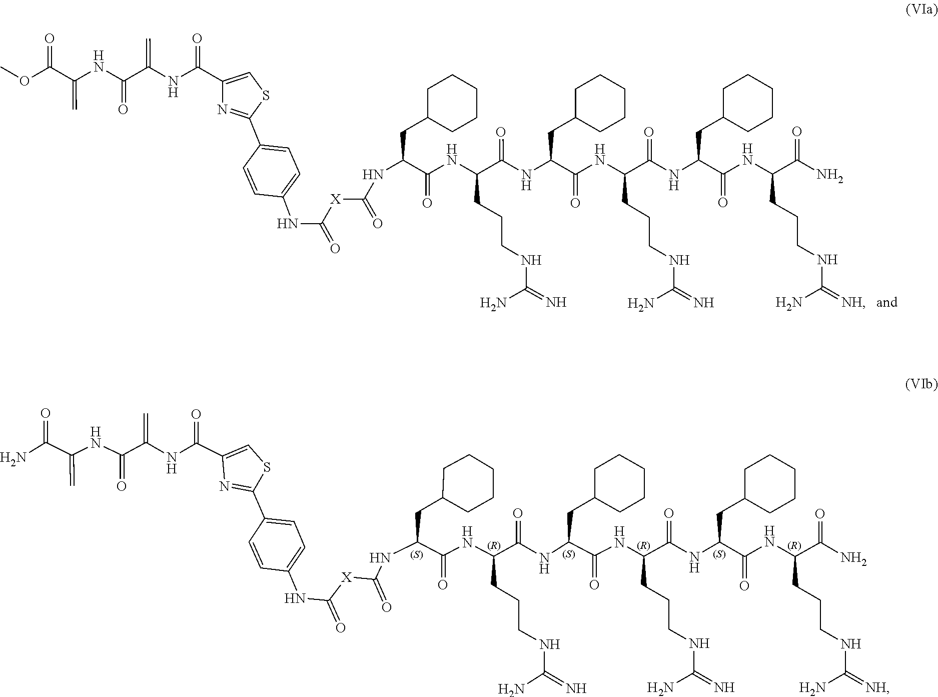

or a pharmaceutically acceptable salt thereof; wherein X is [—(CH₂)ₙ—] or [—(CH₂CH₂—O—)ₘ— (CH₂CH₂)—]; n is 3, 4 or 5; and m is 2, 3, 4, 5, or 6.

In certain embodiments, the present invention provides a pharmaceutically acceptable composition comprising any of the compounds described herein; and a pharmaceutically acceptable carrier.

In certain embodiments, the composition is formulated for oral or parenteral delivery.

In certain embodiments, the compound is contained within a nanoparticle, liposome or micelle, wherein the nanoparticle, liposome, or micelle is conjugated to a mitochondrial targeting moiety.

In certain embodiments, the present invention discloses a composition comprising a compound of Formula (IA) or any subformula thereof, wherein R⁴ is hydrogen, a protecting group, or —C(O)—CH₃, or any of the more specific embodiments thereof described herein contained within a nanoparticle, liposome or micelle, wherein the nanoparticle, liposome, or micelle is conjugated to a mitochondrial targeting moiety.

In certain embodiments, the composition is formulated for oral or parenteral delivery.

In certain embodiments, the present invention discloses a composition comprising a compound of Formula (IB), wherein R⁴ is hydrogen, a protecting group, or —C(O)—CH₃, or any of the more specific embodiments thereof described herein contained within a nanoparticle, liposome or micelle, wherein the nanoparticle, liposome, or micelle is conjugated to a mitochondrial targeting moiety.

In certain embodiments, the nanoparticle, liposome or micelle is selected from poly(ethylene glycol), poly(ε-caprolactone), polysaccharides, poly[(2-hydroxypropyl)-methacrylic acid], poly(lactic-co-glycolic acid), and any combinations of the foregoing. In certain embodiments, the present invention discloses a method of treating a cancer (e.g., solid tumor or hematological cancer) comprising administering to a subject in need thereof a therapeutically effective amount of any of the compounds described herein, or a composition of that compound. In certain embodiments, the cancer (solid tumor or hematological) is selected from lung, breast, prostate, melanoma, esophageal, leukemia, cervical, liver, colon, gastric, colorectal, glioblastoma, head and neck, pancreatic, mesothelioma, and ovarian. In certain embodiments, the cancer is selected from mesothelioma, lung, ovarian, and breast.

In certain embodiments, the present invention discloses a compound selected from:

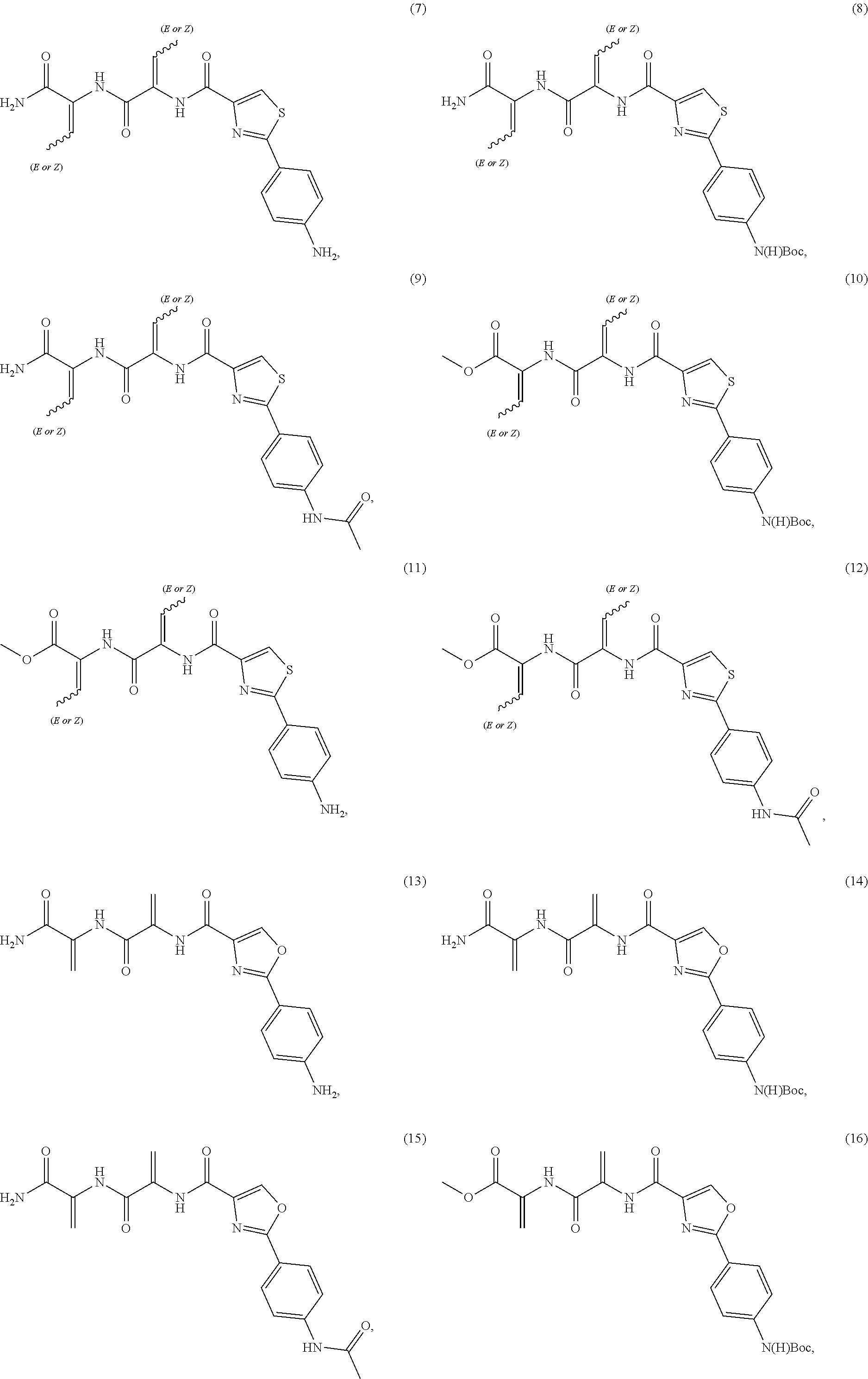

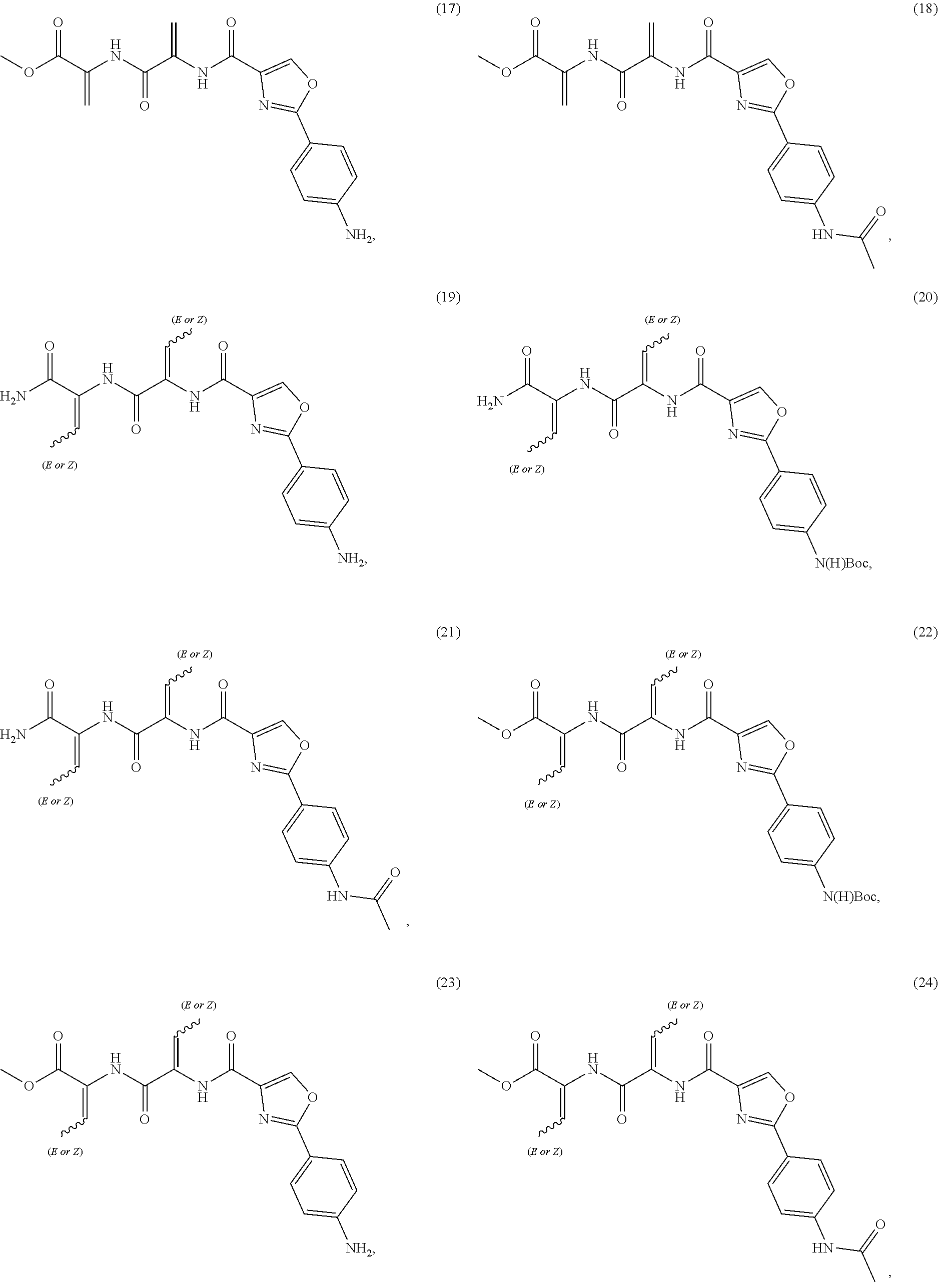

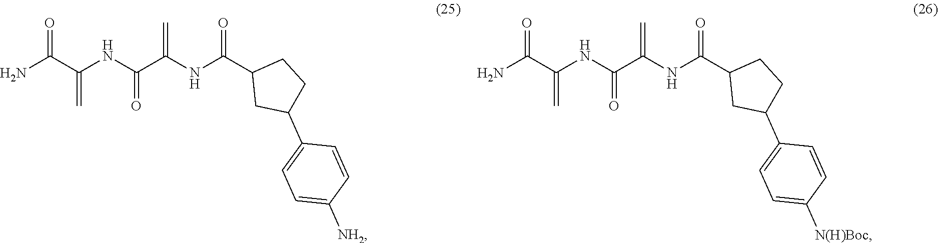

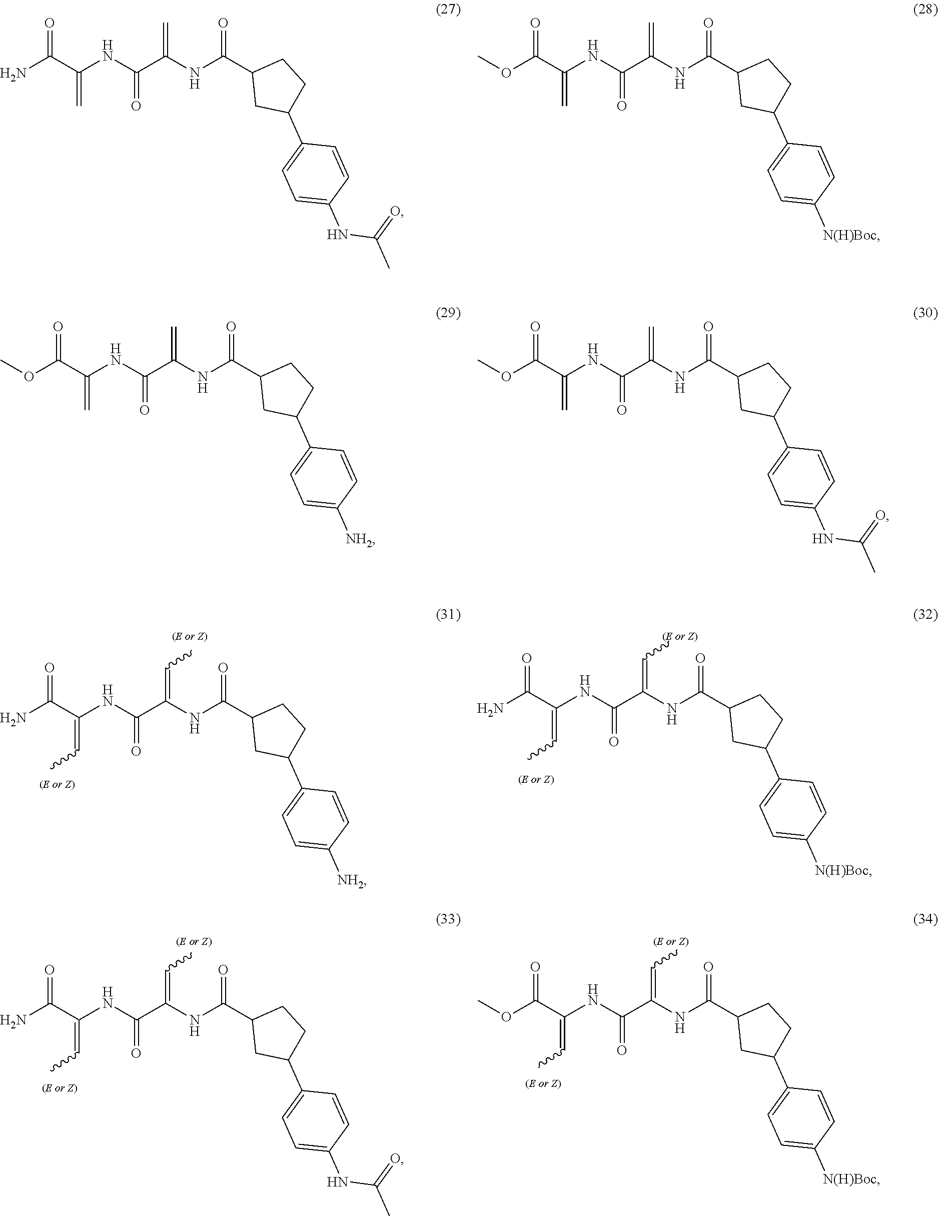

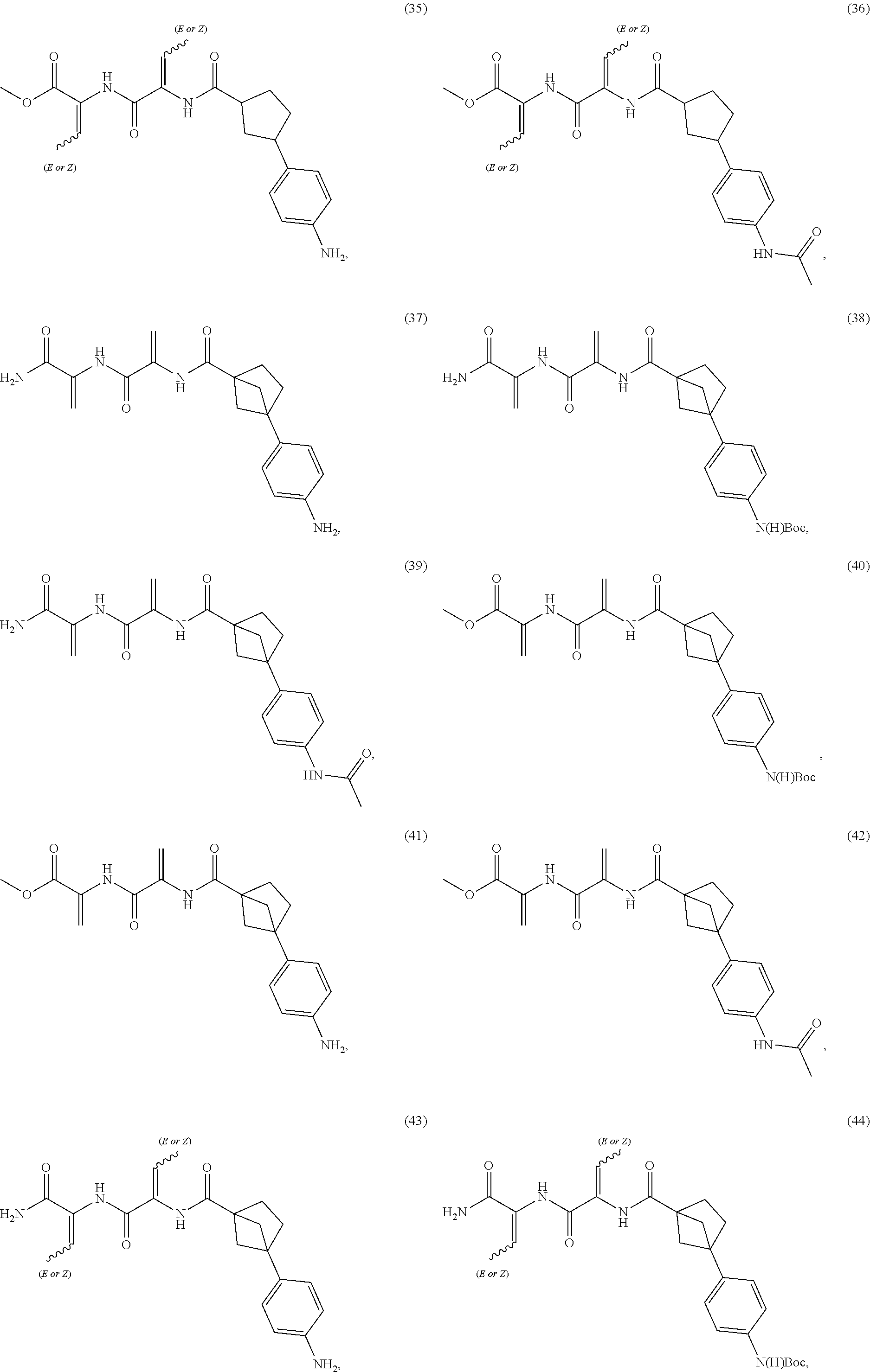

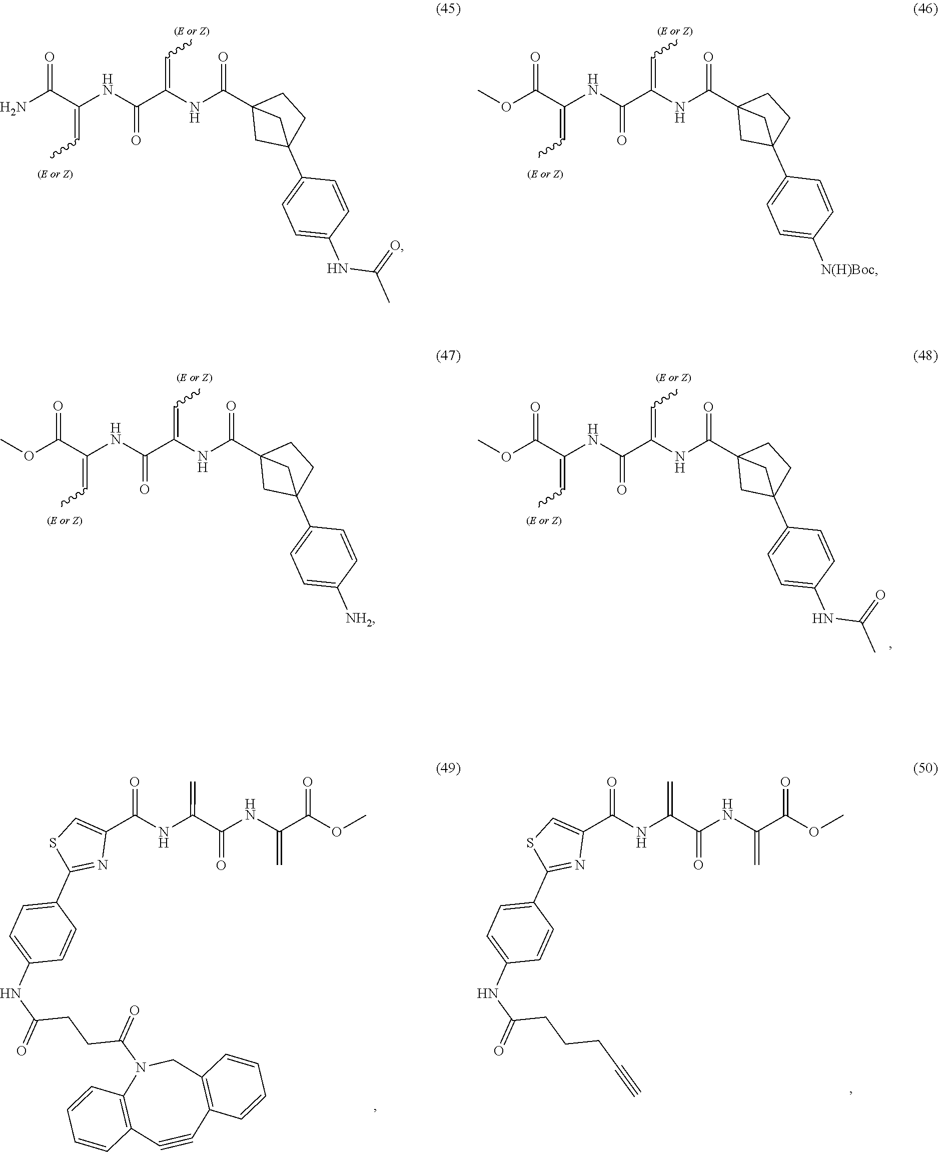

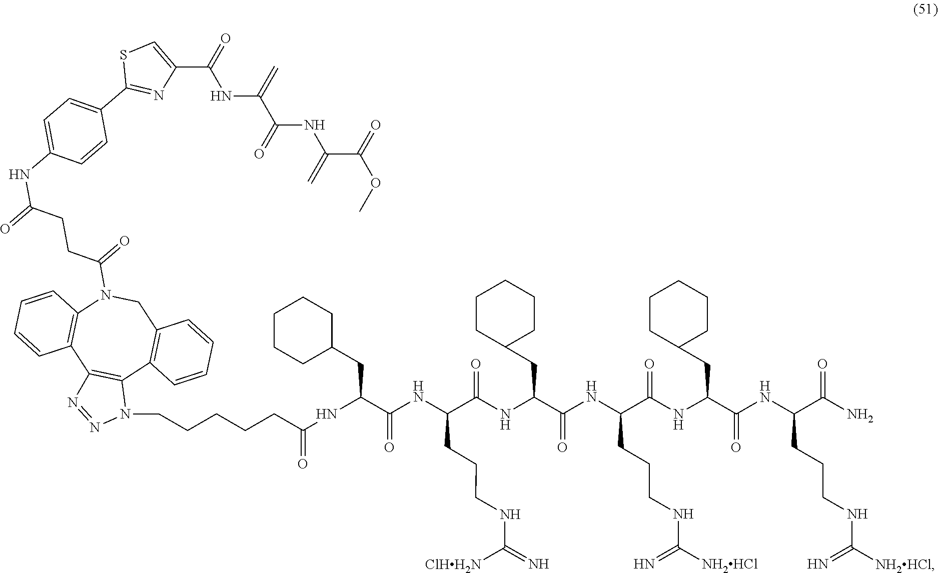

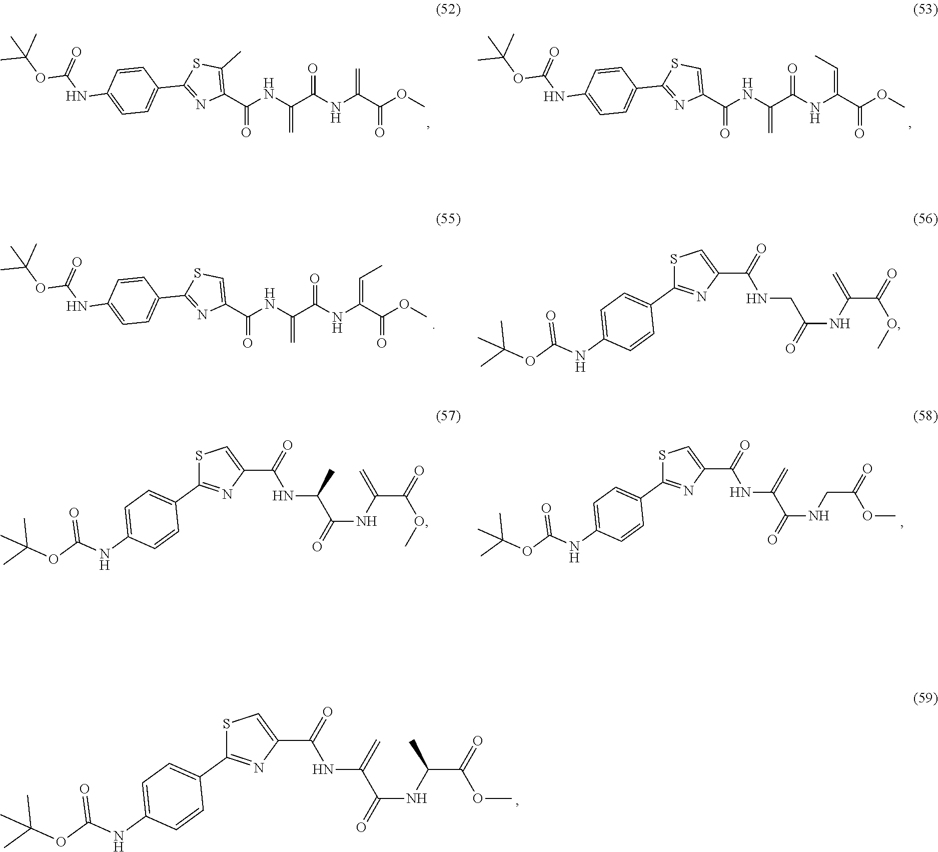

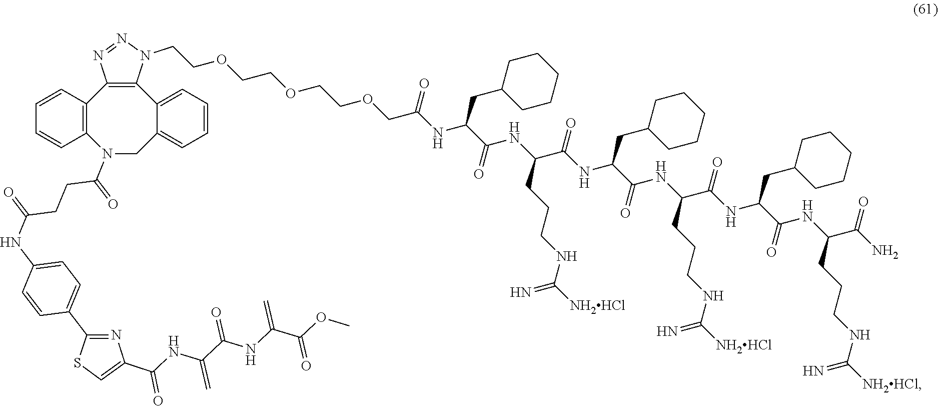

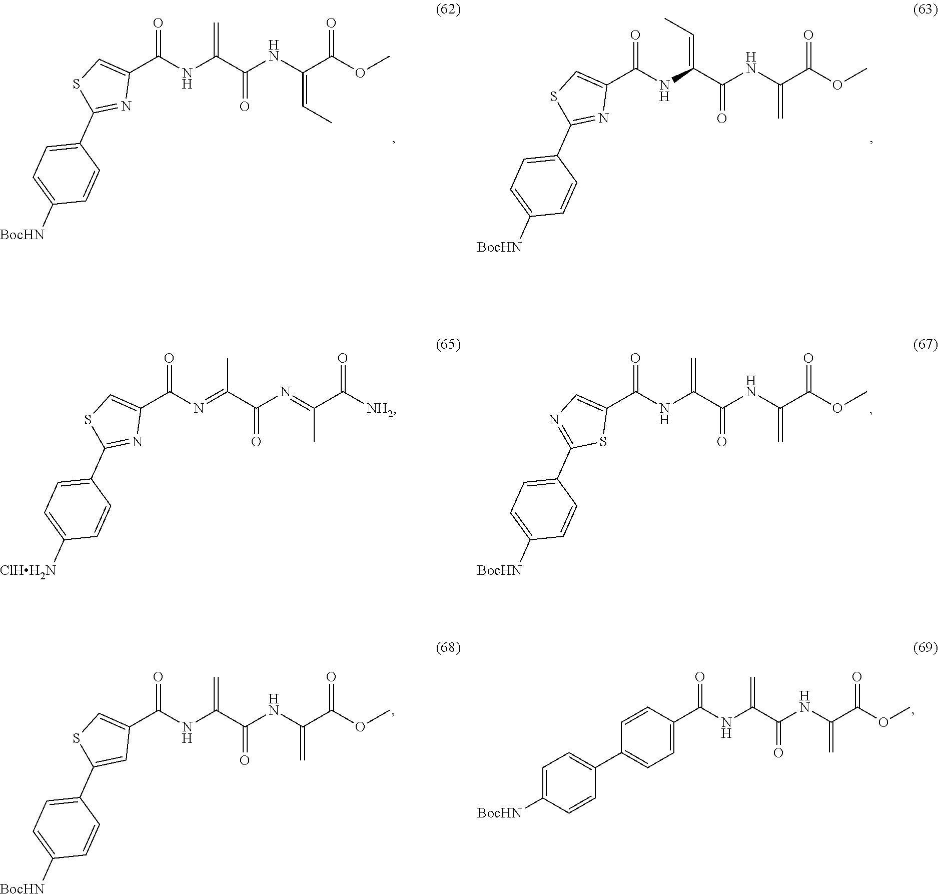

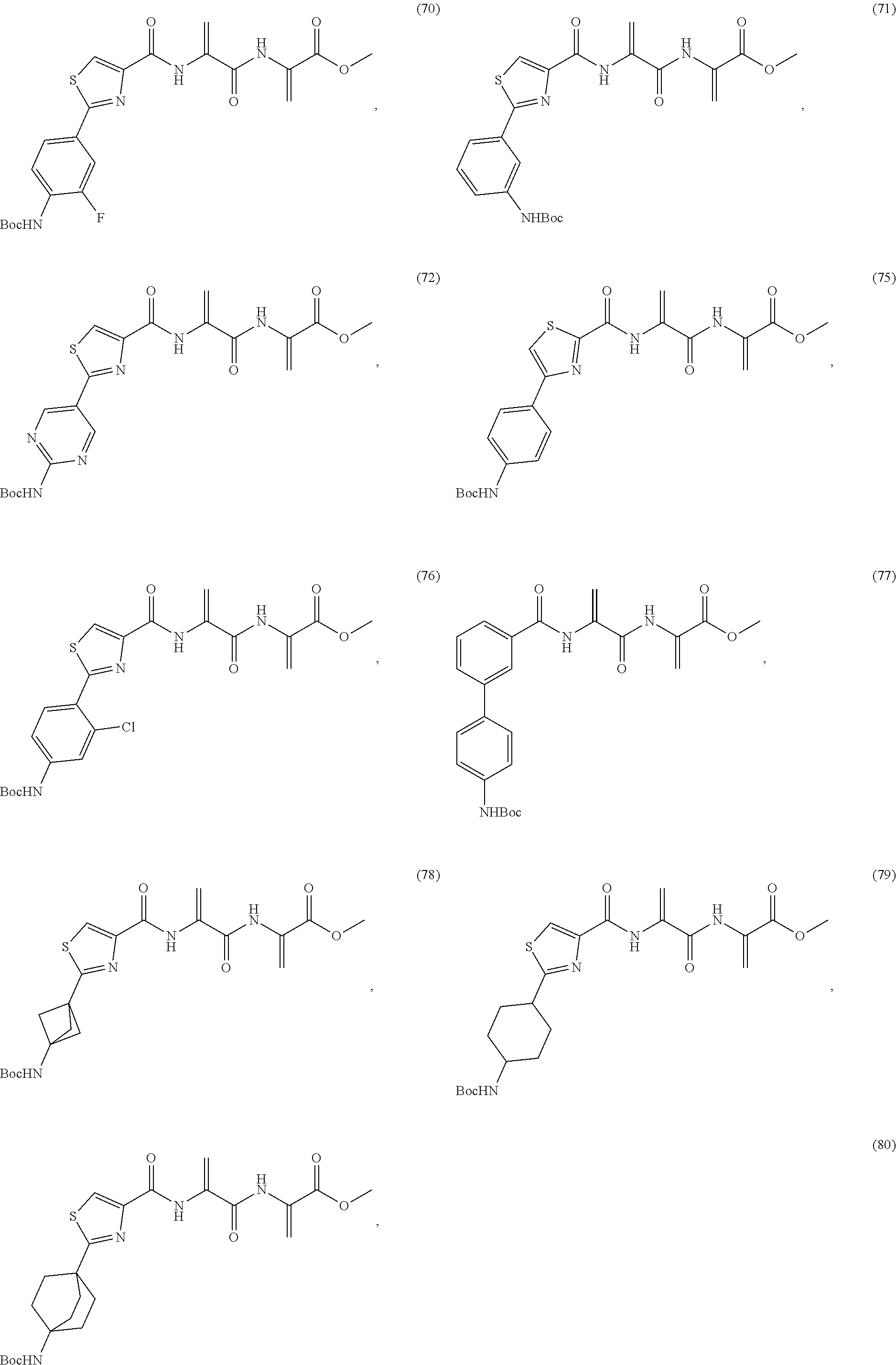

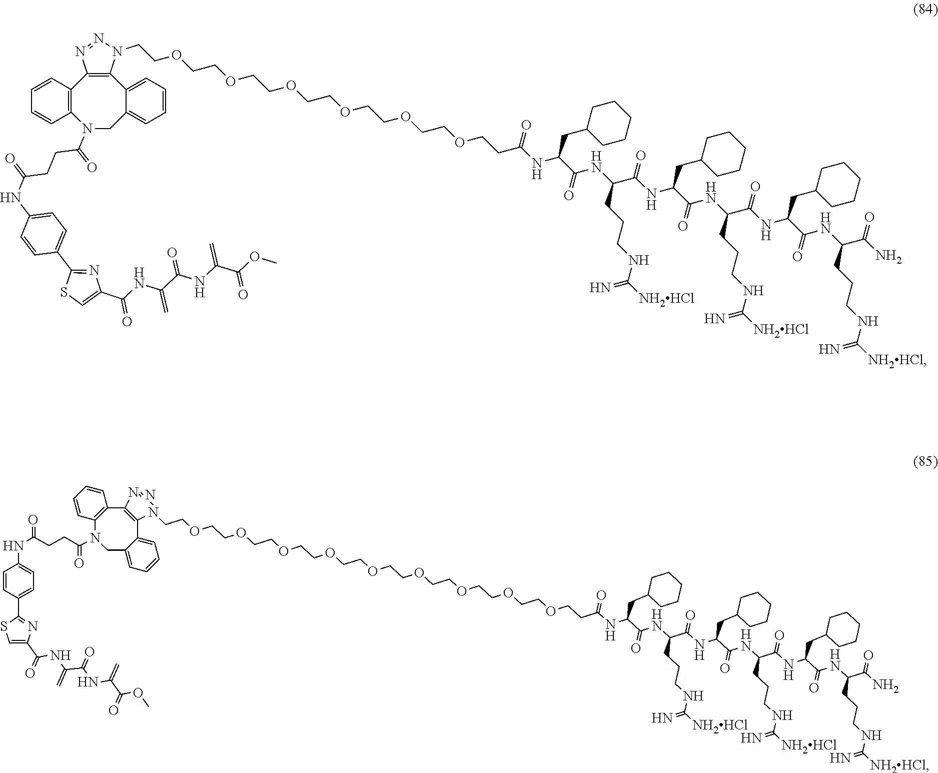

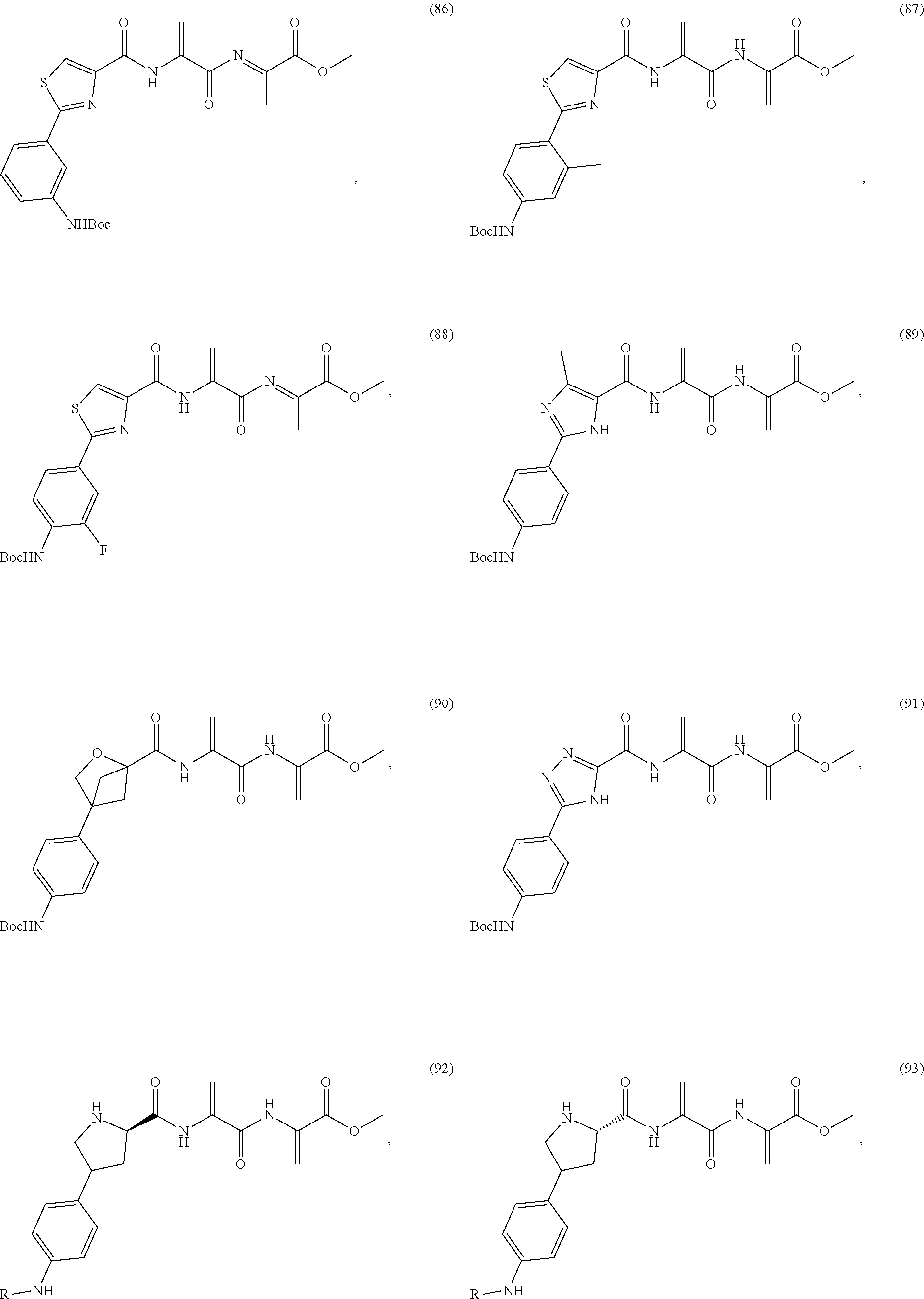

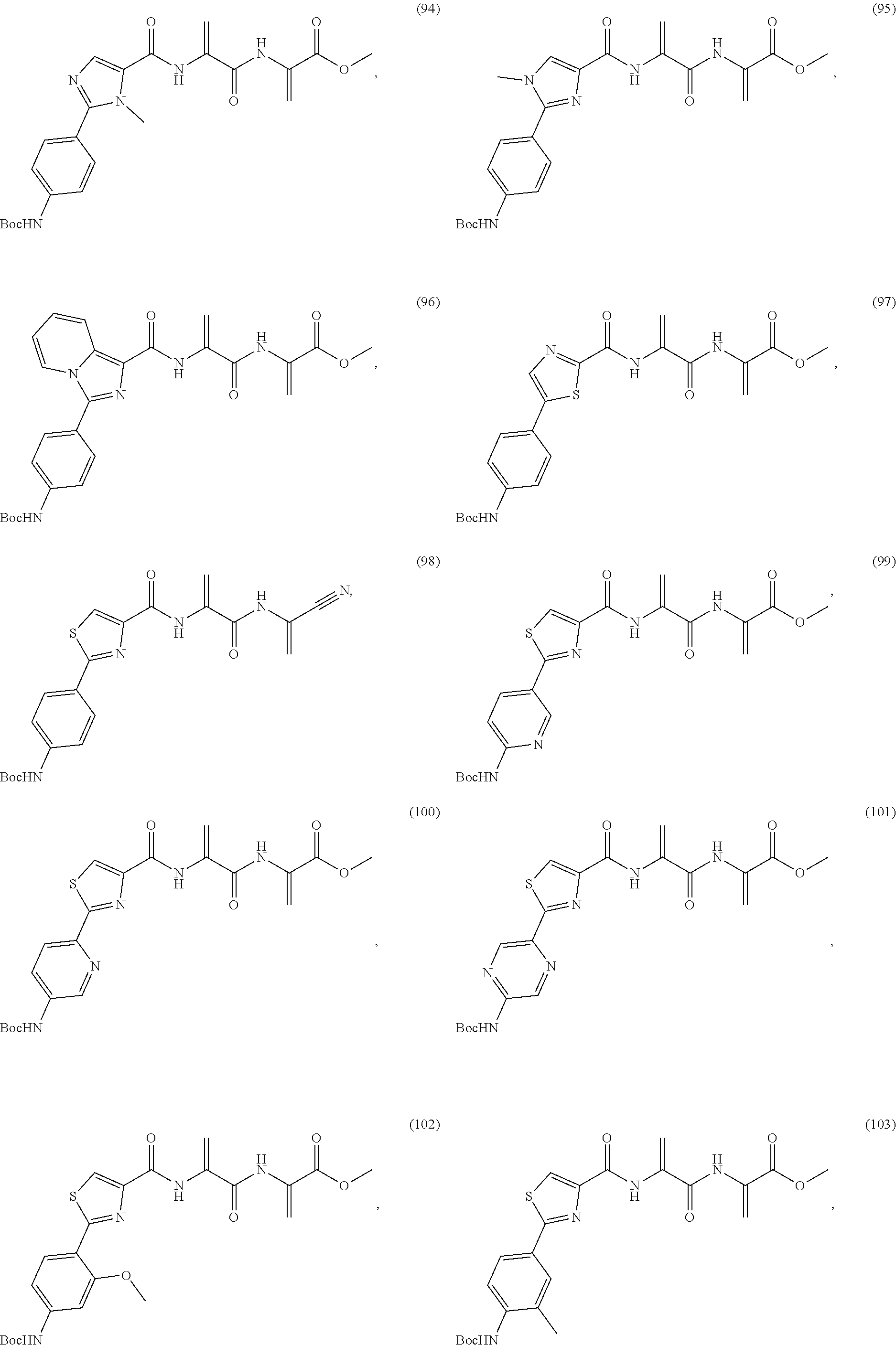

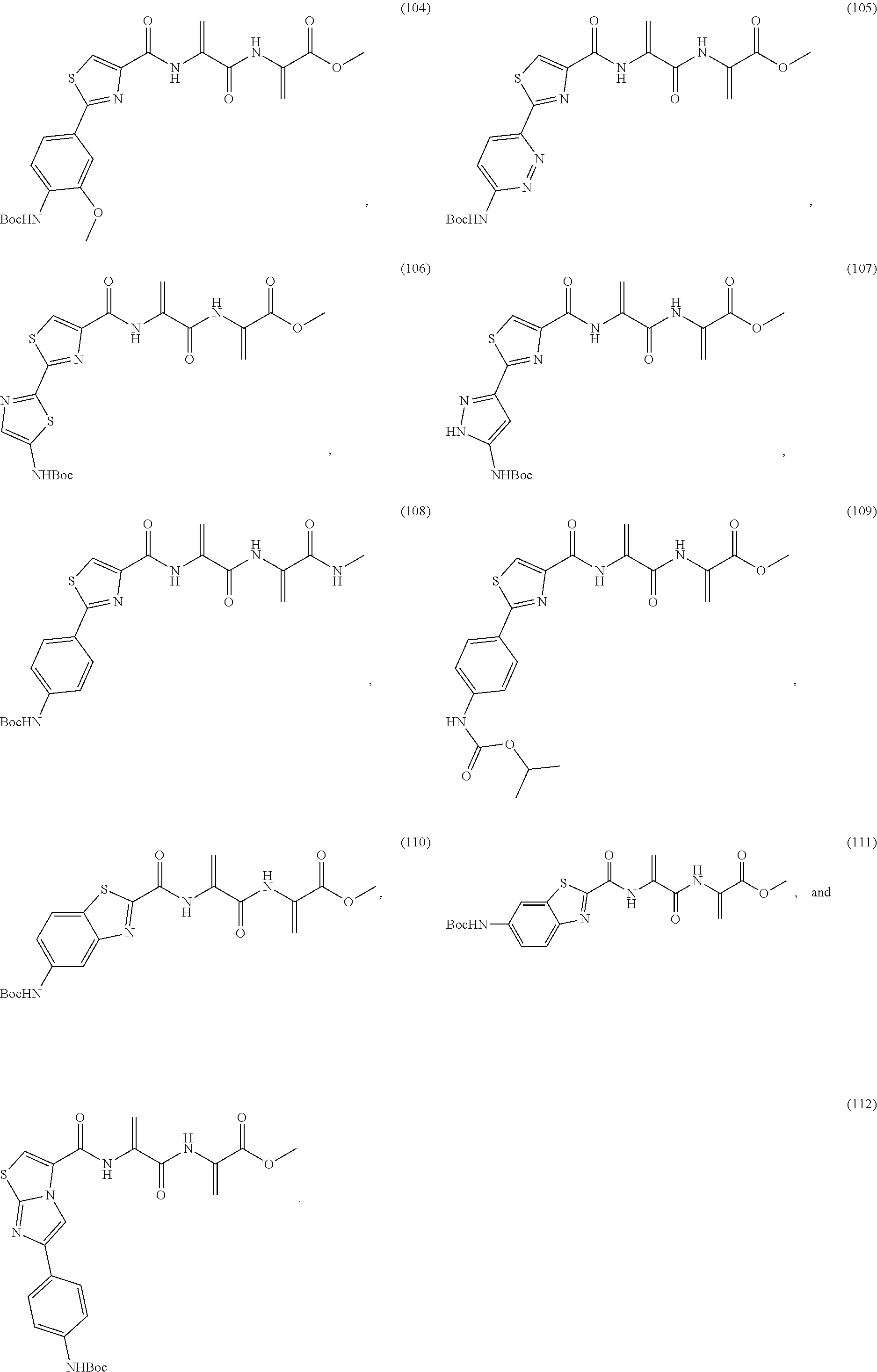

### EXEMPLARY EMBODIMENT EXAMPLES

### General Experimental Procedure 1: Suzuki Coupling

Starting boronic acid or boronic ester (1 eq.) was introduced into a flask under N₂. 1,2-Dimethoxyethane (0.1 M), bromide (1.05 Eq), sodium carbonate 2 M aqueous solution (5 eq.) were added. The mixture was then degassed with N₂ for a couple of minutes. Then palladiumtetrakis (0.05 eq.) was added and the mixture was stirred at 90° C. (ext) until full conversion (overnight). The reaction was then stopped. Water was added, and the mixture was extracted with EtOAc. Brine was added to improve separation. Water was added, and the mixtures was extracted with EtOAc. Brine was added to improve separation. The organic layer was dried over Na₂SO₄, filtered, and concentrated. The crude material was purified by automated FCC. Desired products were obtained with 40-70% yield. Note: in some reactions the hydrolyzed product was also observed. This was isolated by acidifying the aqueous phase and extracted with EtOAc twice. Combined organics were washed with brine, dried over Na₂SO₄, filtered, and concentrated to provide the hydrolyzed product.

### General Experimental Procedure 2: Ester Hydrolysis

To a solution of ester (1 eq.) in THE (0.05 M) was added Lithium hydroxide monohydrate (4 eq.) as solution in water (2M) and the mixture was stirred for 2-16 hours at room temperature. The mixture was diluted EtOAc and washed with 1 M HCl and brine (30 mL each), dried over Na₂SO₄, filtered, and concentrated to provide the desired product.

### General Experimental Procedure 3: Edci Mediated Amide Coupling

To a solution of acid (1 eq.) and amine (1 eq.) in DCM (0.05 M) were added DiPEA (3 eq.), cyanic (E)-2-(hydroxyimino)butanoic anhydride (1.3 eq.) and EDCI (1.3 eq) and the resulting mixture was stirred at room temperature for 16 hours The mixture was washed with 1M HCl, water, NaHCO₃ and brine, dried over Na₂SO₄, filtered, and concentrated. The crude material was purified by automated FCC to afford the desired product.

### General Experimental Procedure 4: Hatu Mediated Amide Coupling

To a solution of acid (1 eq.) and amine (1 eq.) in DCM (0.05 M) were added DiPEA (3 eq.) and HATU (1.2 eq.) and the resulting mixture was stirred at room temperature for 2-16 hours. The mixture was washed with 1M HCl, water, NaHCO₃ and brine, dried over Na₂SO₄, filtered, and concentrated. The crude material was purified by automated FCC to afford the desired product.

### General Experimental Procedure 5: Acetylation of Serine Sidechains

To a solution of the free alcohol (1 eq.) in MeCN (0.05 M) were added triethylamine (2 eq./alcohol), DMAP (0.2 eq) and acetic anhydride (1.1 eq/alcohol) and the resulting mixture was stirred for 2 hours. The reaction mixture was diluted with water and the MeCN was removed in vacuo. The mixture was extracted with EtOAc and the organic phase was washed with brine, dried over Na₂SO₄, filtered, and concentrated. The crude material was purified by automated FCC to afford the desired product.

### General Experimental Procedure 6: Stepwise Elimination of Mono-Tbdps Protected Bisserine

To a solution of mono-TBDPS protected bisserine derivative (1 eq.) in DCM (0.05 M) was added triethylamine (10 eq.) and methane sulfonyl chloride (1.2 eq.) and the resulting mixture was stirred at room temperature for 1 hour. The reaction was followed by LCMS. Optionally, additional triethylamine was added. Upon completion, a solution of tetrabutylammonium fluoride in THE (1 M, 2 eq.) was added and the mixture was stirred for 1 hour. The mixture was washed with 1M HCl and brine, dried over Na₂SO₄, filtered, and concentrated. The residue was taken up in CH₂Cl₂ and triethylamine (10 eq) and methane sulfonyl chloride (1.2 eq.) were added. The resulting mixture was stirred for 1 hour at room temperature. The reaction was followed by LCMS. Optionally, additional triethylamine was added. The mixture was washed with 1M HCl and brine, dried over Na₂SO₄, filtered, and concentrated. The crude material was purified by automated reverse phase FCC.

### General Experimental Procedure 7: Stepwise Elimination of Mono-Tbdps, Mono-Acetate

To a solution of mono-TBDPS mono-acetyl bisserine derivative (1 eq.) in CH₂Cl₂ (0.05 M) was added DBU (2 eq.) and the mixture was stirred at room temperature for 1-16 h. Then a solution of TBAF in THE (1 M, 2 eq.) was added and the mixture stirred for 2 hours. The mixture was concentrated and purified by automated FCC. To a solution of the mono-eliminated intermediate in CH₂Cl₂ (0.05 M) were added triethylamine (6 eq) and methanesulfonyl chloride (1.5 eq.). Optionally, additional triethylamine was added. The mixture was washed with 1M HCl and brine, dried over Na₂SO₄, filtered, and concentrated. The crude material was purified by automated reverse phase FCC.

### General Experimental Procedure 8: Elimination of Bisacetate

A solution of the bisacetate in THF/CH₂Cl₂ (1/1, v/v, 0.05 M) was cooled to 0° C. and DBU (4 eq.) was added. The mixture was stirred at 0° C. for 2 hours. The mixture was diluted with CH₂C₂ and water and HCl (aq 1M) were added to acidify the mixture to pH=4. The mixture was extracted with CH₂Cl₂ (3x) and the combined organic layers were washed with brine, dried over Na₂SO₄, filtered, and concentrated. The crude material was purified by automated reverse phase FCC.

### General Experimental Procedure 9: Preparation of Primary Amide

To a solution of carboxylic acid (1 eq.) in DMF (0.1 M) or CH₂Cl₂ (0.15 M) were added NH₄C₁ (3 eq.), DiPEA (3.5 eq) and HATU (1.1 eq). The mixture was stirred at room temperature overnight. The reaction was quenched with water. In case of clear solution, then the mixture was extracted with EtOAc. The organic layer was washed with brine, dried over Na₂SO₄, filtered and concentrated. The crude material was purified by automated FCC. In case of precipitate, then the mixture was filtered and washed with water and the solvent used in the reaction. The solid was recovered, concentrated to remove the traces of solvent, and afforded the desired primary amide.

### General Experimental Procedure 10: Thiation Primary Amide

To a solution of primary amide (1 eq.) in THE (0.2 M) was added Lawesson's reagent (0.7 eq.) and the mixture was stirred at 80° C. (ext.) until full conversion. Then the reaction was quenched with NaHCO₃sat. solution and the mixture was extracted with EtOAc. The organic layer was washed with brine, dried over Na₂SO₄, filtered and concentrated. The crude material was purified by automated FCC.

General Experimental Procedure 11: Thiazole Formation from Thioamide

To a solution of primary thioamide (1 eq.) in EtOH (0.2 M) was added ethyl bromopyruvate (1.1 eq.) and the resulting mixture was stirred at room temperature or 50° C. (ext.) until full conversion. The reaction was quenched with NaHCO₃sat. solution and the mixture was extracted with EtOAc The organic layer was washed with brine, dried over Na₂SO₄, filtered and concentrated. The crude material was purified by automated FCC.

### Analysis Methods

LCMS Method “21020335B TFA LCMS-5 C3”:
System: Agilent 1290 series with UV detector and HP 6130 MSD mass detector
Column: Waters XBridge BEH Cl8 XP (2.1×50 mm; 2.5 μm)
Mobile phase A: 0.05% TFA in Milli-Q
Mobile phase B: Acetonitrile
Pump Flow: 0.6 ml/min
UV Detection: 215.8 nm
Injection Volume: 0.1 μL
Run Time: 3.5 min
Column Temperature: 35° C.
Pump Program: Gradient

| Time (min) | % A | % B |
|---|---|---|
| 0.0 | 95 | 5 |
| 0.5 | 95 | 5 |
| 2.5 | 10 | 90 |
| 3.5 | 10 | 90 |

LCMS Method “21020335C TFA LCMS-5 C₄”:
System: Agilent 1290 series with UV detector and HP 6130 MSD mass detector
Column: Waters XBridge BEH Cl8 XP (2.1×50 mm; 2.5 μm)
Mobile phase A: 0.05% TFA in Milli-Q
Mobile phase B: Acetonitrile
Pump Flow: 0.6 ml/min
UV Detection: 215 8 nm
Injection Volume: 0.1 μL
Run Time: 3.0 min
Column Temperature: 35° C.
Pump Program: Gradient

| Time (min) | % A | % B |
|---|---|---|
| 0.0 | 80 | 20 |
| 1.5 | 10 | 90 |
| 3.0 | 10 | 90 |

LCMS Method “22010199 LCMS-5 C3”:
System: Agilent 1290 series with UV detector and HP 6130 MSD mass detector
Column: Waters XBridge BEH C₁₈ XP (2.1×50 mm; 2.5 μm)
Mobile phase A: 10 mM NH₄₀Ac (Water/Methanol/Acetonitrile 900/60/40)
Mobile phase B: 10 mM NH₄₀Ac (Water/Methanol/Acetonitrile 100/540/360)
Pump Flow: 0.6 ml/min
UV Detection: 215 8 nm
Injection Volume: 0.3 μL
Run Time: 3.5 min
Column Temperature: 35° C.
Pump Program: Gradient

| Time (min) | % A | % B |
|---|---|---|
| 0.0 | 80 | 20 |
| 1.5 | 0 | 100 |
| 3.0 | 0 | 100 |

LCMS Method “22010199A TFA LCMS-5 C₁”:
System: Agilent 1290 series with UV detector and HP 6130 MSD mass detector
Column: Waters Acquity HSS T3 (2.1×75 mm; 1.8 μm)
Mobile phase A: 0.05% TFA in Milli-Q
Mobile phase B: Acetonitrile
Pump Flow: 0.6 ml/min
UV Detection: 215 8 nm
Injection Volume: 0.8 μL
Run Time: 3.0 min
Column Temperature: 35° C.
Pump Program: Gradient

| Time (min) | % A | % B |
|---|---|---|
| 0.0 | 50 | 50 |
| 1.5 | 10 | 90 |
| 3.0 | 10 | 90 |

LCMS Method “22010199C TFA LCMS-5 C3”:
System: Agilent 1290 series with UV detector and HP 6130 MSD mass detector
Column: Waters XBridge BEH C₁₈ XP (2.1×50 mm; 2.5 μm)
Mobile phase A: 0.05% TFA in Milli-Q
Mobile phase B: Acetonitrile
Pump Flow: 0.6 ml/min
UV Detection: 215 8 nm
Injection Volume: 0.8 μL
Run Time: 3.0 min
Column Temperature: 35° C.
Pump Program: Gradient

| Time (min) | % A | % B |
|---|---|---|
| 0.0 | 80 | 20 |
| 1.5 | 10 | 90 |
| 3.0 | 10 | 90 |

LCMS Method “22010199D TFA LCMS-5 C8”:
System: Agilent 1290 series with UV detector and HP 6130 MSD mass detector
Column: Zorbax SB-C8 (2.1×50 mm; 1.8 μm)
Mobile phase A: 0.05% TFA in Milli-Q
Mobile phase B: Acetonitrile
Pump Flow: 0.6 ml/min
UV Detection: 215 8 nm
Injection Volume: 0.1 μL
Run Time: 3.0 min
Column Temperature: 35° C.
Pump Program: Gradient

| Time (min) | % A | % B |
|---|---|---|
| 0.0 | 80 | 20 |
| 1.5 | 10 | 90 |
| 3.0 | 10 | 90 |

LCMS Method “22010199D TFA LCMS-5 C₈”:
System: Agilent 1290 series with UV detector and HP 6130 MSD mass detector
Column: Zorbax SB-C₈ (2.1×50 mm; 1.8 μm)
Mobile phase A: 0.05% TFA in Milli-Q
Mobile phase B: Acetonitrile
Pump Flow: 0.6 ml/min
UV Detection: 215 8 nm
Injection Volume: 0.1 μL
Run Time: 3.0 min
Column Temperature: 35° C.
Pump Program: Gradient

| Time (min) | % A | % B |
|---|---|---|
| 0.0 | 80 | 20 |
| 1.5 | 10 | 90 |
| 3.0 | 10 | 90 |

LCMS Method “30833 LCMS-6”:
System: Agilent 1260 series with UV detector, ELSD 1260 detector and Agilent 6120 mass detector
Column: Waters XBridge BEH C18 XP (2.1×50 mm; 2.5 um)
Mobile phase A: 0.1% TFA in Milli-Q
Mobile phase B: 0.1% TFA in Acetonitrile
Pump Flow: 0.6 ml/min
UV Detection: 215.8 nm
Injection Volume: 0.6 μL
Run Time: 6.0 min
Column Temperature: 35° C.
Pump Program: Gradient

| Time (min) | % A | % B |
|---|---|---|
| 0.0 | 80 | 20 |
| 0.5 | 80 | 20 |
| 3.5 | 10 | 90 |
| 6.0 | 10 | 90 |

LCMS Method “General 3 basic”:
System: Waters Acquity UPLC with PDA detector and QDA mass detector
Column: Waters XBridge BEH C18 (2.1×50 mm; 2.5 μm)
Mobile phase A: 10 mM ammonium bicarbonate in water (pH 9.5)
Mobile phase B: Acetonitrile
Pump Flow: 0.6 ml/min
UV Detection: λmax
Injection Volume: 0.4 μL
Run Time: 2.5 min
Column Temperature: 30° C.
Pump Program: Gradient

| Time (min) | % A | % B |
|---|---|---|
| 0.0 | 80 | 20 |
| 1.5 | 5 | 95 |
| 2.5 | 5 | 95 |
| 2.6 | 85 | 15 |

LCMS Method “General 3 acidic”:
System: Waters Acquity UPLC with PDA detector and QDA mass detector
Column: Waters XBridge BEH C18 (2.1×50 mm; 2.5 μm)
Mobile phase A: 0.1% (v/v) Trifluoroacetic acid in water
Mobile phase B: Acetonitrile
Pump Flow: 0.6 ml/min
UV Detection: λmax
Injection Volume: 0.4 μl
Run Time: 2.5 min
Column Temperature: 30° C.
Pump Program: Gradient

| Time (min) | % A | % B |
|---|---|---|
| 0.0 | 80 | 20 |
| 1.5 | 5 | 95 |
| 2.5 | 5 | 95 |
| 2.6 | 85 | 15 |

### Example 1A: Exemplary Syntheses of Thiostrepton-Inspired Structure A1

### Scheme 1A: A Synthesis Towards Compound Compound A-1

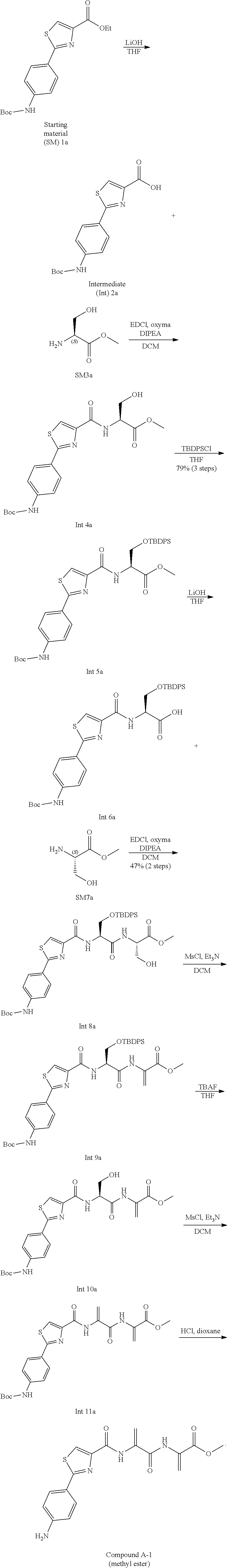

Commercially available thiazole ethyl ester SM1a was hydrolyzed using LiOH to provide carboxylic acid Int 2a. Subsequent amide coupling with serine methyl ester (SM3) provided amide Int 4a. The free hydroxyl group was protected by treatment with TBDPS-C₁ to provide compound Int 5a after column chromatography (79% ver 3 steps). Hydrolysis of the methyl ester using LiGH provided carboxylic acid derivative Int 6a, which was subsequently coupled with serine methyl ester SM7a to provide dipeptide Int 8a, purified by column chromatography (47% ver 2 steps). Elimination of the hydroxyl moiety was achieved by treatment with mesyl chloride and NEt₃ and provided dehydroalanine derivative Int 9a. TBAF mediated TBDPS-removal followed by a second elimination reaction provided bisdehydroalanine derivative Int 11a.

### Example 1B: Exemplary Syntheses of Thiostrepton-Inspired Structure A1 ((5)—HCl)

### Scheme IB: A Synthesis Towards Compound A-1

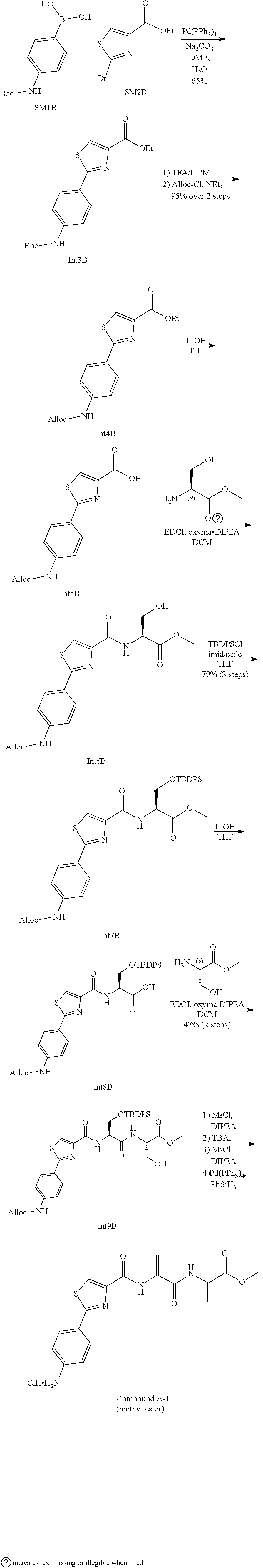

Suzuki coupling between boronic ester SM1B and bromide SM2B provided compound Int3B in moderate yield (650%). The Boc-protecting group was swapped to Alloc in 9500 yield to provide Alloc protected compound Int4B. Hydrolysis of the methyl ester and subsequent EDCI mediated coupling of serine methyl ester provided compound Int6B. The free alcohol was protected with TBDPS and after purification compound Int7B was isolated in 790% ver 3 steps. Hydrolysis ofthe methyl ester and subsequent EDCI mediated coupling with serine methyl ester provided compound Int9B (450% yield). Elimination of the hydroxyl was achieved by treatment with mesyl chloride and NEt₃. TBAF mediated TBDPS-removal followed by a second elimination reaction provided bisdehydroalanine motive. Finally, the Alloc group was removed by treatment with tetrakis in presence of a scavenger to provide Compound A-1.

### Alternate synthesis: 2-(4-((tert-butoxycarbonyl)amino)phenyl)thiazole-4-carboxylic acid Intermediate 2

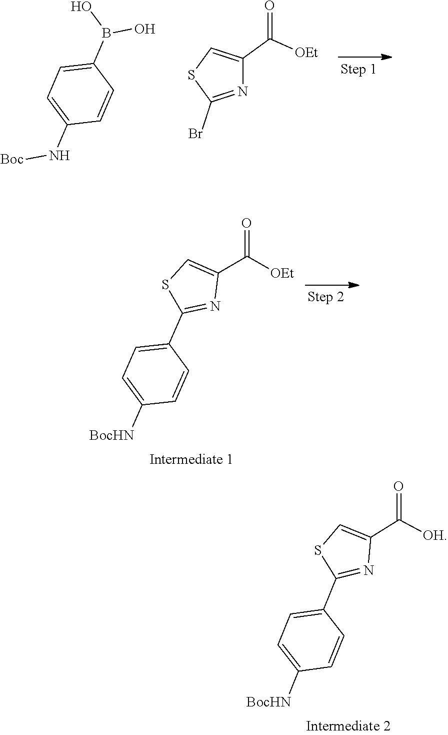 [SMILES: CC(C)(C)OC(=O)Nc1ccc(B(O)O)cc1 ; CCOC(=O)c1csc(Br)n1 ; CCOC(=O)c1csc(-c2ccc(NC(=O)OC(C)(C)C)cc2)n1 ; CC(C)(C)OC(=O)Nc1ccc(-c2nc(C(=O)O)cs2)cc1]

Step 1. Ethyl 2-(4-((tert-butoxycarbonyl)amino)phenyl)thiazole-4-carboxylate (Intermediate 1) was prepared following General experimental procedure 1.4-(tert-butoxycarbonylamino)phenylboronic acid (2.5 g, 11 mmol) and ethyl 2-bromothiazole-4-carboxylate (2.5 g, 11 mmol) gave ethyl 2-(4-((tert-butoxycarbonyl)amino)phenyl)thiazole-4-carboxylate (Intermediate 1) (2.4 g, 6.9 mmol, 65%) as an off-white solid. ¹H NMR (400 MHz, CDCl₃) δ 8.08 (s, 1H), 7.96-7.88 (m, 2H), 7.47-7.40 (m, 2H), 6.59 (s, 1H), 4.42 (q, J=7.1 Hz, 2H), 1.51 (s, 9H), 1.41 (t, J=7.1 Hz, 3H).

Step 2.2-(4-((tert-butoxycarbonyl)amino)phenyl)thiazole-4-carboxylic acid (Intermediate 2) was prepared following General experimental procedure 2.2-(4-((tert-butoxycarbonyl)amino)phenyl)thiazole-4-carboxylate (Intermediate 1) (0.93 g, 2.7 mmol) gave 2-(4-((tert-butoxycarbonyl)amino)phenyl)thiazole-4-carboxylic acid (Intermediate 2) (0.82 g, 2.6 mmol, 97%) as an off-white solid. ¹H NMR (400 MHz, CDCl₃) δ 8.09 (s, 1H), 7.89-7.82 (m, 2H), 7.45-7.34 (m, 2H), 1.47 (s, 9H), 1.24-1.13 (m, 1H).

### Methyl 2-(2-(2-(4-aminophenyl)thiazole-4-carboxamido)acrylamido)acrylate hydrochloride (Compound A1)

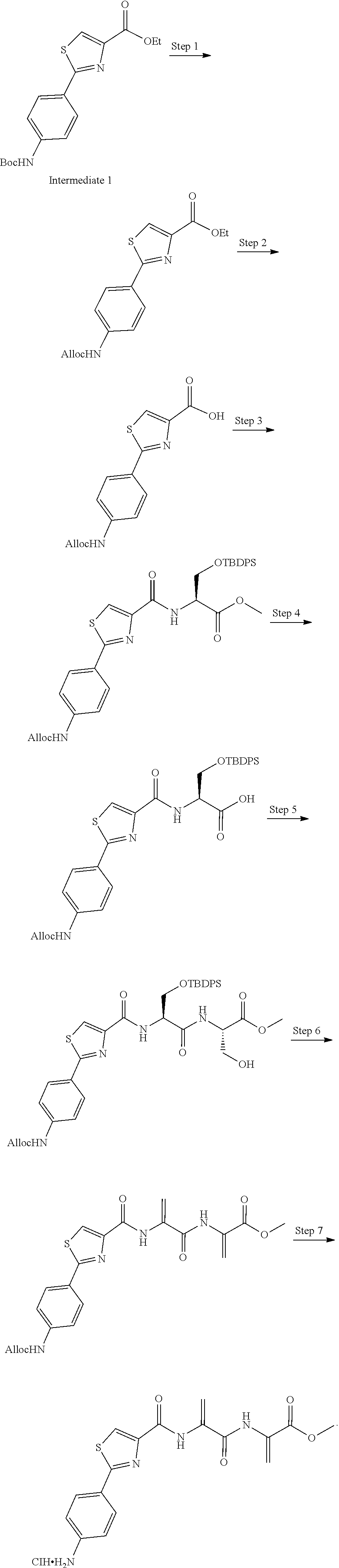

Step 1. Ethyl 2-(4-(((allyloxy)carbonyl)amino)phenyl)thiazole-4-carboxylate was prepared with the following procedure. To a solution of ethyl 2-(4-((tert-butoxycarbonyl)amino)phenyl)thiazole-4-carboxylate (Intermediate 1) (2.4 g, 1 eq., 6.9 mmol) in CH₂Cl₂ (10 mL) was added TFA (10 mL) and the resulting mixture was stirred at room temperature for 2 hours. The mixture was concentrated in vacuo and the residue was taken up in THE (50 mL). To the mixture was added pyridine (2.8 mL, 5 eq., 34 mmol) followed by allyl chloroformate (0.96 mL, 1.3 eq., 9.0 mmol) and the resulting mixture was stirred for 1 hour. The mixture was diluted with EtOAc, washed with 1 M HCl and brine, dried over Na₂SO₄, and concentrated to provide crude ethyl 2-(4-(((allyloxy)carbonyl)amino)phenyl)thiazole-4-carboxylate as a yellow solid. The crude material was used as such in the next step.

Step 2. 2-(4-(((allyloxy)carbonyl)amino)phenyl)thiazole-4-carboxylic acid was prepared following General experimental procedure 2. Crude ethyl 2-(4-(((allyloxy)carbonyl)amino)phenyl)thiazole-4-carboxylate gave 2-(4-(((allyloxy)carbonyl)amino)phenyl)thiazole-4-carboxylic acid (2.0 g, 6.6 mmol, 95%) as orange solid. ¹H NMR (400 MHz, CDCl₃) δ 8.20 (s, 1H), 7.95-7.87 (m, 2H), 7.50 (d, J=8.5 Hz, 2H), 6.83 (s, 1H), 5.96 (ddt, J=17.2, 10.4, 5.8 Hz, 1H), 5.37 (dq, J=17.2, 1.5 Hz, 1H), 5.32-5.24 (m, 1H), 4.68 (dt, J=5.8, 1.4 Hz, 2H).

Step 3. Methyl N-(2-(4-(((allyloxy)carbonyl)amino)phenyl)thiazole-4-carbonyl)-O-(tert-butyldiphenylsilyl)-L-serinate was prepared following General experimental procedure 3. 2-(4-(((allyloxy)carbonyl)amino)phenyl)thiazole-4-carboxylic acid 2-(4-(((allyloxy)carbonyl)amino)phenyl)thiazole-4-carboxylic acid (1.1 g, 1 eq., 3.6 mmol) and methyl O-(tert-butyldiphenylsilyl)-L-serinate hydrochloride (1.7 g, 1.2 eq., 4.3 mmol) gave methyl N-(2-(4-(((allyloxy)carbonyl)amino)phenyl)thiazole-4-carbonyl)-O-(tert-butyldiphenylsilyl)-L-serinate (1.9 g, 3.0 mmol, 82%).

Step 4. N-(2-(4-(((allyloxy)carbonyl)amino)phenyl)thiazole-4-carbonyl)-O-(tert-butyldiphenylsilyl)-L-serine was prepared following General experimental procedure 2. Methyl N-(2-(4-(((allyloxy)carbonyl)amino)phenyl)thiazole-4-carbonyl)-O-(tert-butyldiphenylsilyl)-L-serinate (1.9 g, 1 eq., 3.0 mmol) gave N-(2-(4-(((allyloxy)carbonyl)amino)phenyl)thiazole-4-carbonyl)-O-(tert-butyldiphenylsilyl)-L-serine (1.8 g, 2.8 mmol, 93%) as a clear oil. The crude material was used as such in the next step.

Step 5. Methyl N-(2-(4-(((allyloxy)carbonyl)amino)phenyl)thiazole-4-carbonyl)-O-(tert-butyldiphenylsilyl)-L-seryl-L-serinate was prepared following General experimental procedure 3. N-(2-(4-(((allyloxy)carbonyl)amino)phenyl)thiazole-4-carbonyl)-O-(tert-butyldiphenylsilyl)-L-serine (1.8 g, 2.8 mmol) gave methyl N-(2-(4-(((allyloxy)carbonyl)amino)phenyl)thiazole-4-carbonyl)-O-(tert-butyldiphenylsilyl)-L-seryl-L-serinate (0.75 g, 1.0 mmol, 35%) as a yellow oil.

Step 6. Methyl 2-(2-(2-(4-(((allyloxy)carbonyl)amino)phenyl)thiazole-4-carboxamido)acrylamido)acrylate was prepared following General experimental procedure 6. Methyl N-(2-(4-(((allyloxy)carbonyl)amino)phenyl)thiazole-4-carbonyl)-O-(tert-butyldiphenylsilyl)-L-seryl-L-serinate (0.75 g, 1 eq., 1.0 mmol) gave crude methyl 2-(2-(2-(4-(((allyloxy)carbonyl)amino)phenyl)thiazole-4-carboxamido)acrylamido)acrylate (0.9 g) which was used without purification in the next step.

Step 7. Methyl 2-(2-(2-(4-aminophenyl)thiazole-4-carboxamido)acrylamido)acrylate was prepared with the following procedure. To a solution of crude methyl 2-(2-(2-(4-(((allyloxy)carbonyl)amino)phenyl)thiazole-4-carboxamido)acrylamido)acrylate (0.90 g) in DCM (20 mL) was added phenylsilane (1.1 g, 1.2 mL, 5 eq., 9.9 mmol) followed by tetrakis(triphenylphosphine)-palladium(0) (0.23 g, 0.1 eq., 0.20 mmol) and the resulting mixture was stirred for 1 hour. MeCN (4 mL) was added and the CH₂Cl₂ was removed in vacuo. The mixture was directly submitted to automated reverse phase FCC to provide methyl 2-(2-(2-(4-aminophenyl)thiazole-4-carboxamido)acrylamido)acrylate (8.5 mg, 23 μmol, 1.2%) as an offwhite solid. LCMS (21020335C TFA LCMS-5 C4) RT: 1.274 min; area % (215 nm): 79.1%; m z=373.2 [M+H]⁺. ¹H NMR (400 MHz, MeOD) δ 8.38 (s, 1H), 8.33-8.17 (m, 2H), 7.57-7.52 (m, 2H), 6.60 (d, J=1.8 Hz, 1H), 6.29 (s, 1H), 6.00 (s, 1H), 5.76 (d, J=1.9 Hz, 1H), 3.86 (s, 3H).

### Example 2A: Exemplary Syntheses of Thiostrepton-Inspired Structure A2 ((1))

### Scheme 2A: A Synthesis Towards Compound A-2 Through a Dipeptide Building Block:

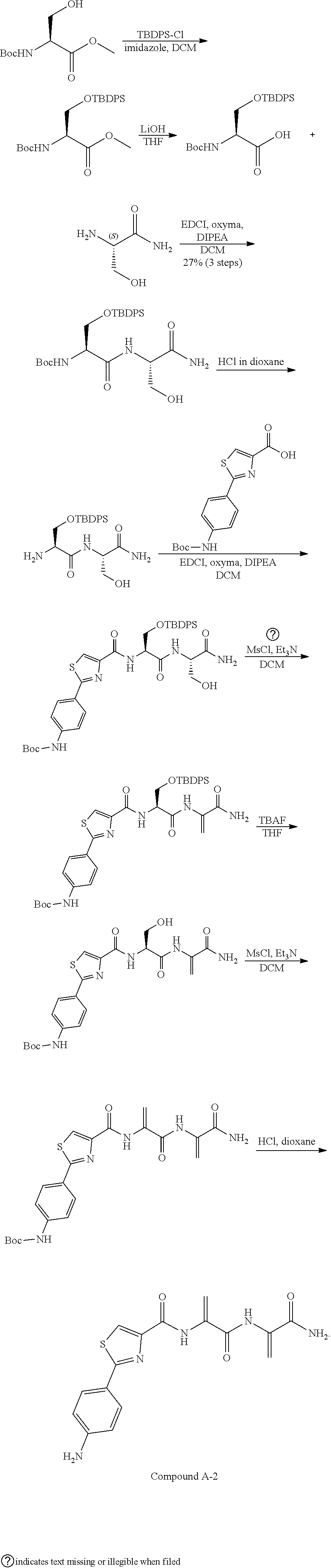

Compound A-2 was accessed by coupling between a thiazole derivative and a dipeptide. The dipeptide was synthesized starting from Boc-Ser-OMe. First the hydroxyl was protected by treatment with TBDPS-Cl to provide a serine derivative. The methyl ester was hydrolyzed by treatment with LiOH to provide a carboxylic acid, which was then coupled with serinamide to provide a dipeptide after column chromatography (27% over 3 steps). The Boc protecting group was removed by treatment with HCl and provided the di peptide. EDCI-mediated coupling of the dipeptide and carboxylic acid provided a thiazole coupled dipeptide after column chromatography (51%). Elimination of the hydroxyl, mediated by mesyl chloride and NEt₃, followed by TBDPS removal by TBAF and a second elimination reaction provided Boc-protected compound after preparative HIPLC purification. Treatment with HCl removed the Boc protecting group and provided Compound A-2.

### Example 2B: Exemplary Syntheses of Thiostrepton-inspired structure A2

Scheme 2B: A synthesis towards Compound A-2 through a dipeptide building block

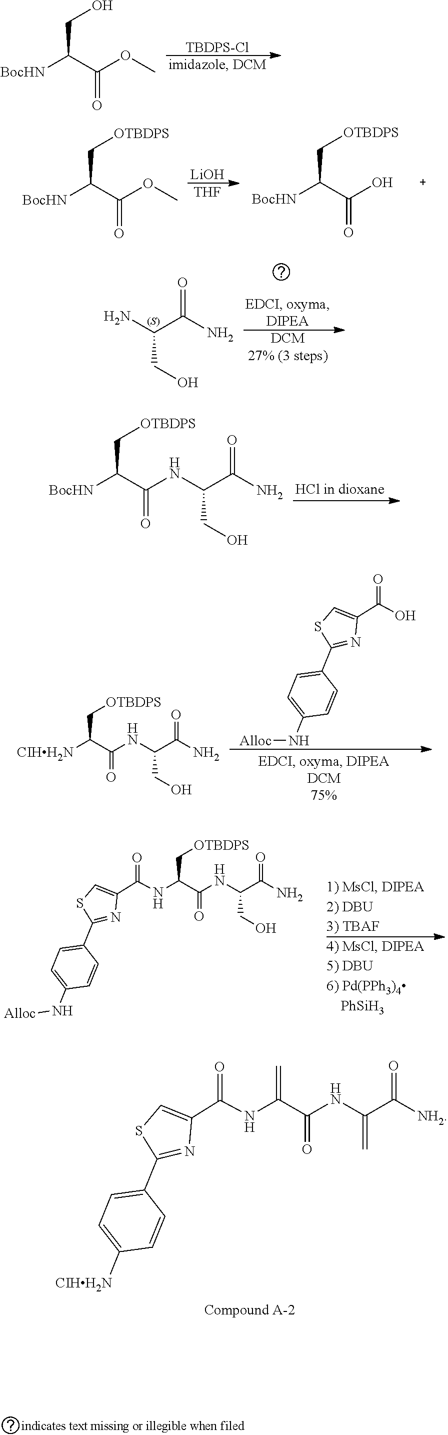

The hydroxyl-moiety of Boc-Ser-OMe was protected with TBDPS to provide Boc-Ser(TBDPS)—OMe. The methyl ester was subsequently removed by treatment with LiOH to provide an acid. EDCI-mediated peptide coupling with serinamide provided a dipeptide in 27% over 3 steps. Treatment with HCl removed the Boc-group to provide an amine. EDCI-mediated coupling between the amine and the acid, synthesis of which is shown above, provided the product in 75% over 2 steps. Elimination of the hydroxyl was achieved by treatment with mesyl chloride and NEt₃. TBAF mediated TBDPS-removal followed by a second elimination reaction provided bisdehydroalanine motive. Finally, the Alloc group was removed by treatment with palladium in presence of a scavenger to provide Compound A-2.

### Alternate synthesis of tautomer of Compound A-2/tautomer ((1)/tautomer): Methyl 2-(2-(2-(4-aminophenyl)thiazole-4-carboxamido)acrylamido)acrylate hydrochloride

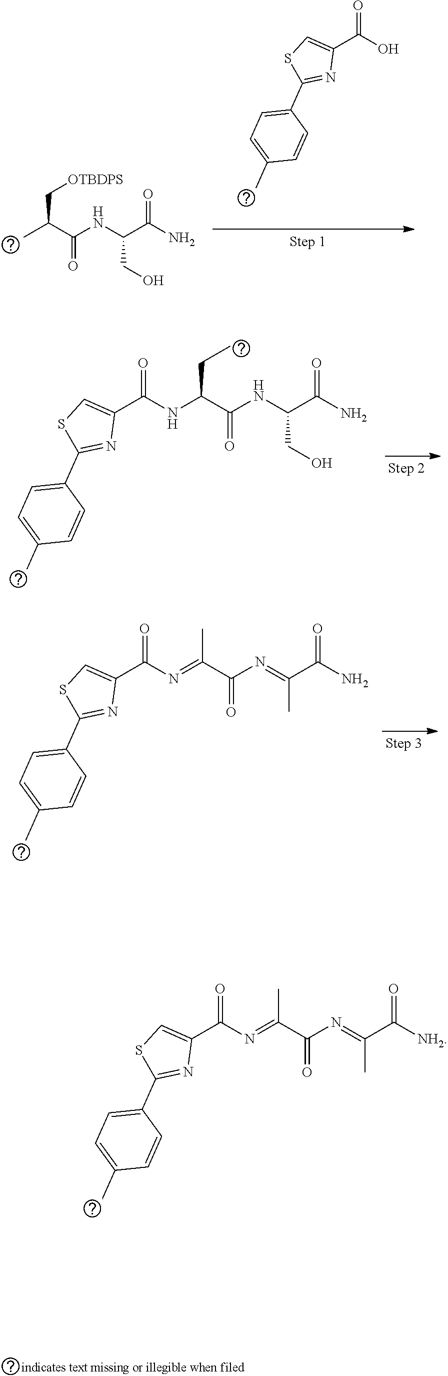

Step 1. allyl (4-(4-(((S)-1-(((S)-1-amino-3-hydroxy-1-oxopropan-2-yl)amino)-3-((tert-butyldiphenylsilyl)oxy)-1-oxopropan-2-yl)carbamoyl)thiazol-2-yl)phenyl)carbamate was prepared following General experimental procedure 3. 2-(4-(((allyloxy)carbonyl)amino)phenyl)thiazole-4-carboxylic acid (1.2 g, 1 eq., 3.8 mmol) (as prepared in step 2) and (S)-2-amino-N —((S)-1-amino-3-hydroxy-1-oxopropan-2-yl)-3-((tert-butyldiphenylsilyl)oxy)propanamide hydrochloride (as prepared in 7) (2.2 g, 1.2 eq., 4.6 mmol) gave allyl (4-(4-(((S)-1-(((S)-1-amino-3-hydroxy-1-oxopropan-2-yl)amino)-3-((tert-butyldiphenylsilyl)oxy)-1-oxopropan-2-yl)carbamoyl)thiazol-2-yl)phenyl)carbamate (1.5 g, 2.1 mmol, 55%) as a white foam. LCMS (General 3 acidic) RT: 1.44 min; area % (254 nm): 92%; m z=716.2 [M+H]⁺. ¹H NMR (400 MHz, CDCl₃) δ 8.35 (d, J=6.6 Hz, 1H), 8.06 (s, 1H), 7.89-7.81 (m, 2H), 7.65 (tt, J=6.2, 1.5 Hz, 4H), 7.38 (dddd, J=22.2, 20.7, 8.4, 6.7 Hz, 8H), 6.85 (s, 1H), 6.79 (s, 1H), 6.03-5.89 (m, 1H), 5.42-5.32 (m, 2H), 5.31-5.23 (m, 1H), 4.71-4.59 (m, 3H), 4.56 (dt, J=7.7, 4.1 Hz, 1H), 4.22 (ddd, J=17.4, 10.8, 3.3 Hz, 2H), 3.95 (dd, J=10.2, 4.9 Hz, 1H), 3.63 (dd, J=11.4, 4.5 Hz, 1H), 3.47 (s, 1H), 1.09 (s, 9H).

Step 2. allyl (4-(4-((3-((3-amino-3-oxoprop-1-en-2-yl)amino)-3-oxoprop-1-en-2-yl)carbamoyl)thiazol-2-yl)phenyl)carbamate was prepared following General experimental procedure 6. allyl (4-(4-(((S)-1-(((S)-1-amino-3-hydroxy-1-oxopropan-2-yl)amino)-3-((tert-butyldiphenylsilyl)oxy)-1-oxopropan-2-yl)carbamoyl)thiazol-2-yl)phenyl)carbamate (0.35 g, 0.49 mmol) gave crude allyl (4-(4-((3-((3-amino-3-oxoprop-1-en-2-yl)amino)-3-oxoprop-1-en-2-yl)carbamoyl)thiazol-2-yl)phenyl)carbamate (0.35 g), which was used as such in the next step.

Step 3. N-(3-((3-amino-3-oxoprop-1-en-2-yl)amino)-3-oxoprop-1-en-2-yl)-2-(4-aminophenyl)thiazole-4-carboxamide hydrochloride was prepared using the following procedure. To a solution of crude allyl (4-(4-((3-((3-amino-3-oxoprop-1-en-2-yl)amino)-3-oxoprop-1-en-2-yl)carbamoyl)thiazol-2-yl)phenyl)carbamate (0.35 g, 0.79 mmol) in DCM (10 mL) were added Silylbenzene (0.49 mL, 5 eq., 4.0 mmol) and tetrakis (92 mg, 0.1 eq., 79 μmol) and the resulting mixture was stirred for 1 hour. MeCN (4 mL) was added and the DCM was removed in vacuo. The mixture was directly submitted to automated reverse phase FCC to provide methyl 2-(2-(2-(4-aminophenyl)thiazole-4-carboxamido)acrylamido)acrylate hydrochloride as a white solid. LCMS (21020335B TFA LCMS-5 C₃) RT: 1.138 min; area: 63.7% (215 nm); m z=458.2 [M+H]⁺.

### Example 3: Exemplary Syntheses of Thiostrepton-inspired structure B

Scheme 3: Synthesis towards compounds hexapeptide Compound B

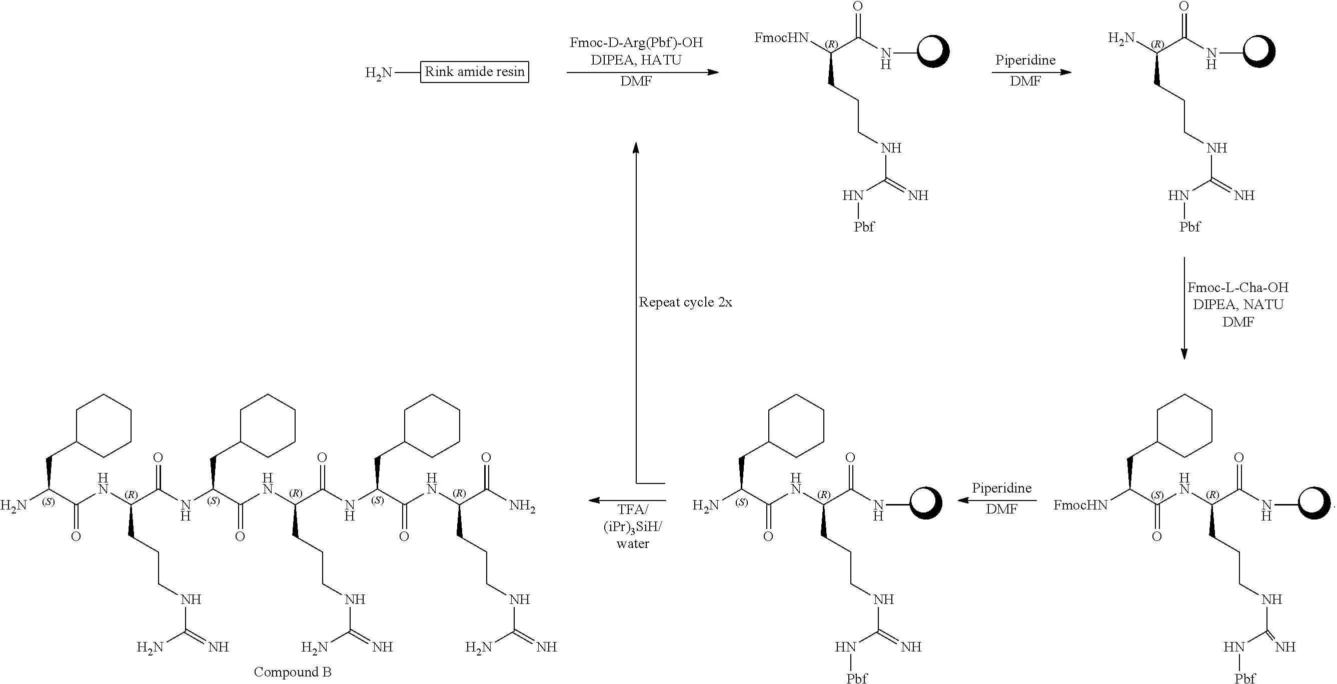

Sequential build-up SPPS ofhexapeptide compound B was started using 5 gram Rink Amide resin (0.7 mmol/g) and using standard SPPS protocols. After full deprotection and simultaneous cleavage from the resin using TFA/TIPS/water (95/2.5/2.5), the crude peptide was obtained by precipitation from MTBE:heptane (1:1) and lyophilization. The crude peptide was purified using preparative HIPLC to provide >9500 purity of Compound B as the TFA salt. To convert to the HCl salt, the purified peptide was basified with NaOH and reacidified with HCl and subsequent preparative HIPLC using HCl buffers provided the pure peptide as HCl salt.

### Alternate Synthesis:

### H-Cha-D-Arg-Cha-D-Arg-Cha-D-Arg-NH₂ HCl salt

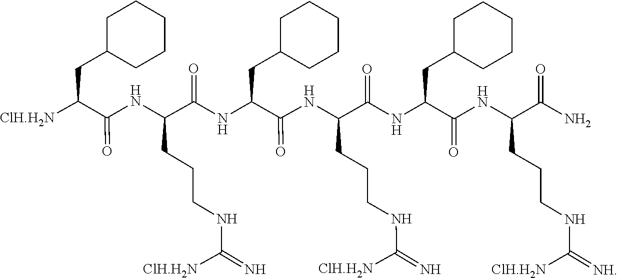

H-Cha-D-Arg-Cha-D-Arg-Cha-D-Arg-NH₂ HCl salt was prepared following General procedure for solid phase peptide synthesis on 2.8 mmol scale. Purification using HCl buffers provided the desired HCl salt form. H-Cha-D-Arg-Cha-D-Arg-Cha-D-Arg-NH₂ HCl salt (0.89 g, 0.82 mmol, 29%) was isolated as a white solid. LCMS (30833 LCMS-6): RT: 1.819 min; Area: 92.5% (215 nm), 99.7% (ELSD); m z=945.9 [M+H]+

### Example 4: Compounds 1 and 2

### tert-butyl (4-(4-((3-((3-amino-3-oxoprop-1-en-2-yl)amino)-3-oxoprop-1-en-2-yl)carbamoyl)thiazol-2-yl)phenyl)carbamate & tert-butyl (4-(4-(((E)-1-(((E)-1-amino-1-oxopropan-2-ylidene)amino)-1-oxopropan-2-ylidene)carbamoyl)thiazol-2-yl)phenyl)carbamate

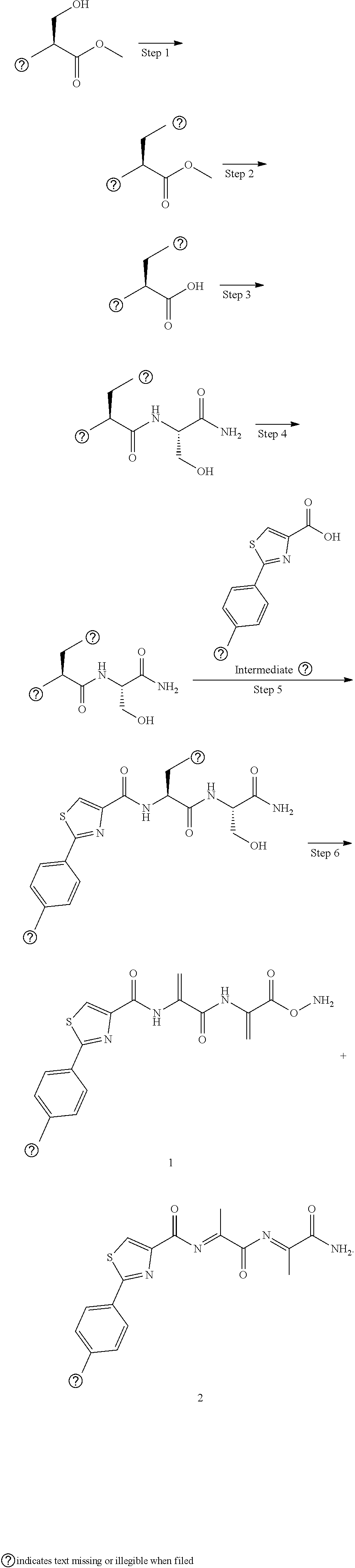

Step 1. Methyl N-(tert-butoxycarbonyl)-O-(tert-butyldiphenylsilyl)-L-serinate was prepared with the following procedure. To a solution of methyl (tert-butoxycarbonyl)-L-serinate (12 g, 1 eq., 55 mmol) in DCM (250 mL) and imidazole (8.2 g, 2.2 eq., 0.12 mol) was added TBDPS-C₁ (17 g, 15 mL, 1.1 eq., 60 mmol) and the resulting mixture was stirred at room temperature overnight. The mixture was washed with 1M HCl (200 mL) and brine (200 mL), dried over Na₂SO₄, filtered, and concentrated to give methyl N-(tert-butoxycarbonyl)-O-(tert-butyldiphenylsilyl)-L-serinate (24 g, 52 mmol, 96%) as cloudy oil, which solidified on standing. ¹H NMR (400 MHz, CDCl₃) δ 7.59 (m, 4H), 7.46-7.32 (m, 6H), 5.39 (d, J=8.8 Hz, 1H), 5.28 (d, J=1.0 Hz, 1H), 4.38 (dt, J=9.2, 3.1 Hz, 1H), 4.05 (dd, J=10.0, 3.0 Hz, 1H), 3.87 (dd, J=10.1, 3.1 Hz, 1H), 3.72 (s, 3H), 1.44 (s, 9H), 1.01 (s, 9H).

Step 2. N-(tert-butoxycarbonyl)-O-(tert-butyldiphenylsilyl)-L-serine was prepared following General experimental procedure 2. Methyl N-(tert-butoxycarbonyl)-O-(tert-butyldiphenylsilyl)-L-serinate (12 g, 26 mmol) gave N-(tert-butoxycarbonyl)-O-(tert-butyldiphenylsilyl)-L-serine (11.9 g, 26.8 mmol, quant.). The material was used in the next step without purification.

Step 3. tert-butyl ((S)-1-(((S)-1-amino-3-hydroxy-1-oxopropan-2-yl)amino)-3-((tert-butyldi phenylsilyl)oxy)-1-oxopropan-2-yl)carbamate was prepared following General experimental procedure 3. N-(tert-butoxycarbonyl)-O-(tert-butyldiphenylsilyl)-L-serine (10 g, 1 eq., 23 mmol) and L-serinamide hydrochloride (6.5 g, 2 eq., 46 mmol) gave tert-butyl ((S)-1-(((S)-1-amino-3-hydroxy-1-oxopropan-2-yl)amino)-3-((tert-butyldiphenylsilyl)oxy)-1-oxopropan-2-yl)carbamate (2.5 g, 4.7 mmol, 20%) as a slightly pink solid. ¹H NMR (400 MHz, CDCl₃) δ 7.59 (m, 4H), 7.48-7.33 (m, 6H), 7.21 (m, 1H), 6.83 (s, 1H), 5.33 (s, 1H), 5.23 (s, 1H), 4.50 (s, 1H), 4.22 (dd, J=11.3, 2.7 Hz, 1H), 4.16 (d, J=5.8 Hz, 1H), 4.09 (dd, J=10.3, 4.1 Hz, 1H), 3.83 (dd, J=10.4, 4.4 Hz, 1H), 3.60 (dd, J=11.4, 4.3 Hz, 1H), 1.43 (s, 9H), 1.04 (s, 9H).

Step 4. (S)-2-amino-N—((S)-1-amino-3-hydroxy-1-oxopropan-2-yl)-3-((tert-butyldiphenylsilyl)oxy)propanamide hydrochloride was prepared with the following procedure. Tert-butyl ((S)-1-(((S)-1-amino-3-hydroxy-1-oxopropan-2-yl)amino)-3-((tert-butyldiphenylsilyl)oxy)-1-oxopropan-2-yl)carbamate (2.5 g, 1 Eq, 4.7 mmol) was dissolved in 15 mL 4 M HCl in dioxane and the resulting mixture was stirred at room temperature for 90 minutes. The mixture was concentrated to provide (S)-2-amino-N—((S)-1-amino-3-hydroxy-1-oxopropan-2-yl)-3-((tert-butyldiphenylsilyl)oxy)propanamide hydrochloride (2.2 g, 4.6 mmol, 98%) as white solid. ¹H NMR (400 MHz, MeOD) δ 7.73-7.62 (m, 4H), 7.52-7.39 (m, 6H), 4.54 (t, J=5.4 Hz, 1H), 4.11 (t, J=4.5 Hz, 1H), 4.03 (d, J=4.5 Hz, 2H), 3.86-3.74 (m, 2H), 1.07 (s, 9H).

Step 5. tert-butyl (4-(4-(((S)-1-(((S)-1-amino-3-hydroxy-1-oxopropan-2-yl)amino)-3-((tert-butyldiphenylsilyl)oxy)-1-oxopropan-2-yl)carbamoyl)thiazol-2-yl)phenyl)carbamate was prepared following General experimental procedure 3. 2-(4-((tert-butoxycarbonyl)amino)phenyl)thiazole-4-carboxylic acid (Intermediate 2) (0.70 g, 2.2 mmol) and (S)-2-amino-N—((S)-1-amino-3-hydroxy-1-oxopropan-2-yl)-3-((tert-butyldiphenylsilyl)oxy)propanamide hydrochloride (2.0 g, 2 eq., 4.4 mmol) gave tert-butyl (4-(4-(((S)-1-(((S)-1-amino-3-hydroxy-1-oxopropan-2-yl)amino)-3-((tert-butyldiphenylsilyl)oxy)-1-oxopropan-2-yl)carbamoyl)thiazol-2-yl)phenyl)carbamate (1.3 g, 1.8 mmol, 81%). %). LCMS (General 3 acidic) RT: 1.59 min; area % (214 nm): 75%; m z=732.2 [M+H]⁺.

Step 6. tert-butyl (4-(4-((3-((3-amino-3-oxoprop-1-en-2-yl)amino)-3-oxoprop-1-en-2-yl)carbamoyl)thiazol-2-yl)phenyl)carbamate & tert-butyl (4-(4-(((E)-1-(((E)-1-amino-1-oxopropan-2-ylidene)amino)-1-oxopropan-2-ylidene)carbamoyl)thiazol-2-yl)phenyl)carbamate were prepared using the following procedure. To a solution of tert-butyl (4-(4-(((S)-1-(((S)-1-amino-3-hydroxy-1-oxopropan-2-yl)amino)-3-((tert-butyldiphenylsilyl)oxy)-1-oxopropan-2-yl)carbamoyl)thiazol-2-yl)phenyl)carbamate (0.13 g, 0.18 mmol) in CH₂C₂ (2 mL) were added triethylamine (0.12 mL, 5 eq., 0.89 mmol) and methansulfonyl chloride (21 μL, 1.5 eq., 0.27 mmol) and the resulting mixture was stirred at room temperature for 3 hours. The mixture was diluted with CH₂C₂ (25 mL) and washed with 1M HCl and brine, dried over Na₂SO₄, filtered and concentrated partially to reach a volume of roughly 10 mL. Then, DBU (54 μL, 2 eq., 0.36 mmol) was added and the mixture was stirred for 1 hour. A solution of TBAF in THE (0.36 mL, 1.0 molar, 2 eq., 0.36 mmol) was added and the mixture was stirred for 30 minutes. The mixture was diluted with CH₂C₃ (25 mL) and washed with 1M HCl and brine, dried over Na₂SO₄, filtered and concentrated partially to reach a volume of roughly 10 mL. To the resulting mixture were added triethylamine (0.12 mL, 5 eq., 0.89 mmol) and methanesulfonyl chloride (21 μL, 1.5 eq., 0.27 mmol) and the resulting mixture was stirred for 1 hour at room temperature. The mixture was diluted with CH₂C₂ (25 mL) and washed with 1M HCl and brine, dried over Na₂SO₄, filtered and concentrated partially to reach a volume of roughly 10 mL. DBU (54 μL, 2 eq., 0.36 mmol) was added and the resulting mixture was stirred for 1 hour. The mixture was diluted with CH₂Ch (25 mL) and washed with 1M HCl and brine, dried over Na₂SO₄, filtered, and concentrated. The crude material was purified by automated reverse phase FCC to provide 1 (2.0 mg, 4.4 μmol, 2.4%) and 2 (8.0 mg, 17 μmol, 9.7%).

1: LCMS (22010199A TFA LCMS-5 Cl) RT: 1.138 min; area: 63.7% (215 nm); m/z 458.2 [M+H]⁺. ¹H NMR (400 MHz, CDCl₃) δ 9.99 (s, 1H), 8.92 (s, 1H), 8.07 (d, J=5.2 Hz, 1H), 7.96-7.86 (m, 2H), 7.45 (m, 2H), 6.76 (d, J=2.2 Hz, 1H), 6.65 (d, J=2. 1 Hz, 1H), 6.62 (s, 1H), 5.53 (t, J=1.9 Hz, 1H), 5.43-5.36 (m, 1H), 1.65 (s, 13H), 1.52 (s, 9H).

2: LCMS (22010199 LCMS-5 C3) RT: 1.138 min; area: 63.7% (215 nm); m/z 458.2 [M+H]⁺. ¹H NMR (400 MHz, CDCl₃) δ 10.84 (s, 1H), 8.22 (s, 1H), 7.93-7.86 (m, 2H), 7.53-7.47 (m, 2H), 7.36 (s, 1H), 6.66 (s, 1H), 2.33 (s, 3H), 1.78 (s, 3H), 1.53 (s, 9H).

### Example 5: Compound 4

### Methyl 2-(2-(2-(4-((tert-butoxycarbonyl)amino)phenyl)thiazole-4-carboxamido)acrylamido)acrylate

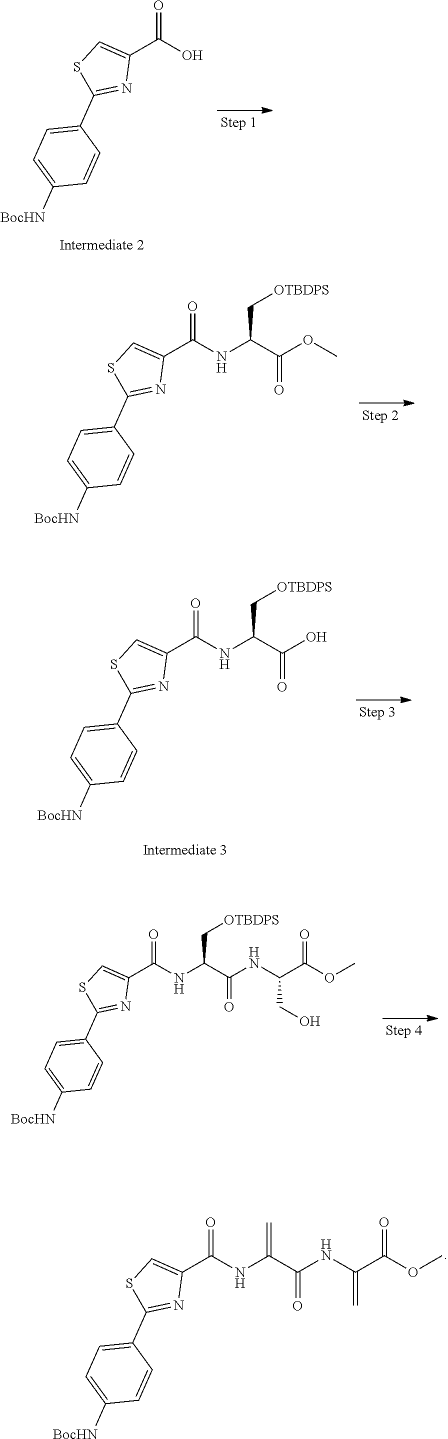

Step 1. Methyl N-(2-(4-((tert-butoxycarbonyl)amino)phenyl)thiazole-4-carbonyl)-O-(tert-butyldiphenylsilyl)-L-serinate was prepared following General experimental procedure 3.2-(4-((tert-butoxycarbonyl)amino)phenyl)thiazole-4-carboxylic acid (Intermediate 2) (2.0 g, 6.2 mmol) and methyl O-(tert-butyldiphenylsilyl)-L-serinate hydrochloride (2.5 g, 1 eq., 6.2 mmol) gave methyl N-(2-(4-((tert-butoxycarbonyl)amino)phenyl)thiazole-4-carbonyl)-O-(tert-butyldiphenylsilyl)-L-serinate (3.5 g, 5.3 mmol, 85%).

Step 2. N-(2-(4-((tert-butoxycarbonyl)amino)phenyl)thiazole-4-carbonyl)-O-(tert-butyldiphenylsilyl)-L-serine (Intermediate 3) was prepared following General experimental procedure 2. Methyl N-(2-(4-((tert-butoxycarbonyl)amino)phenyl)thiazole-4-carbonyl)-O-(tert-butyldiphenylsilyl)-L-serinate (3.5 g, 5.3 mmol) gave crude N-(2-(4-((tert-butoxycarbonyl)amino)phenyl)thiazole-4-carbonyl)-O-(tert-butyldi phenylsilyl)-L-serine (Intermediate 3) as clear oil, which was used as such in the next step.

Step 3. Methyl N-(2-(4-((tert-butoxycarbonyl)amino)phenyl)thiazole-4-carbonyl)-O-(tert-butyldiphenylsilyl)-L-seryl-L-serinate was prepared following General experimental procedure 3. Crude N-(2-(4-((tert-butoxycarbonyl)amino)phenyl)thiazole-4-carbonyl)-O-(tert-butyldiphenylsilyl)-L-serine (Intermediate 3) (as obtained from step 2) and methyl L-serinate hydrochloride (1.2 g, 1.5 eq., 8.0 mmol) gave methyl N-(2-(4-((tert-butoxycarbonyl)amino)phenyl)thiazole-4-carbonyl)-O-(tert-butyldiphenylsilyl)-L-seryl-L-serinate (1.0 g, 1.3 mmol, 25%) as a yellow oil. LCMS (General 3 acidic) RT: 1.74 min; area % (254 nm): 87%; m z=747.2 [M+H]⁺.

Step 4. Methyl 2-(2-(2-(4-((tert-butoxycarbonyl)amino)phenyl)thiazole-4-carboxamido)acrylamido)acrylate was prepared following General experimental procedure 6. Methyl N-(2-(4-((tert-butoxycarbonyl)amino)phenyl)thiazole-4-carbonyl)-O-(tert-butyldiphenylsilyl)-L-seryl-L-serinate (0.10 g, 1 eq., 0.13 mmol) gave methyl 2-(2-(2-(4-((tert-butoxycarbonyl)amino)phenyl)thiazole-4-carboxamido)acrylamido)acrylate (22 mg, 47 μmol, 35%) as a white solid. LCMS (22010199A TFA LCMS-5 Cl) RT: 1.6884 min; area % (215 nm): 97.1%; m z=473.2 [M+H]⁺. ¹H NMR (400 MHz, CDCl₃) δ 9.98 (s, 1H), 8.52 (s, 1H), 8.07 (s, 1H), 7.96-7.88 (m, 2H), 7.45 (d, J=8.7 Hz, 2H), 6.75 (d, J=2.2 Hz, 1H), 6.69 (s, 1H), 6.61 (s, 1H), 6.01 (d, J=1.3 Hz, 1H), 5.48 (t, J=1.9 Hz, 1H), 3.88 (s, 3H), 1.52 (s, 9H).

### Alternate Synthesis:

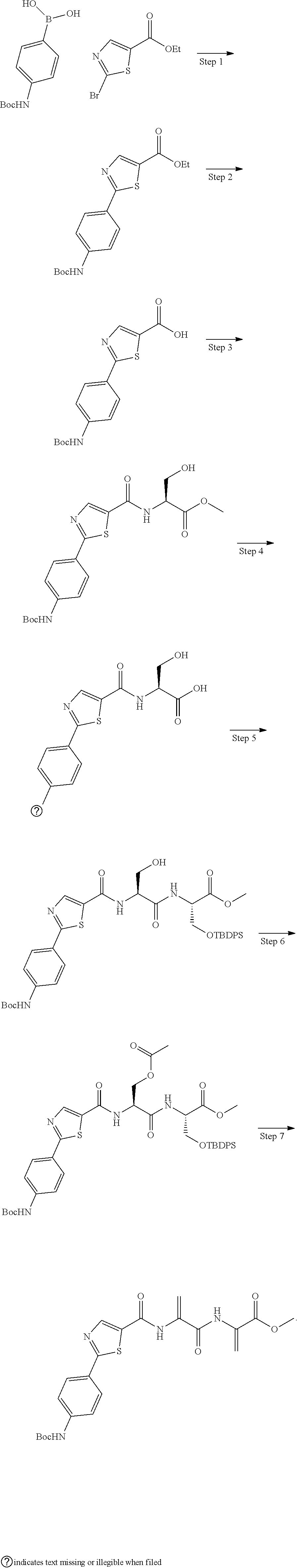

Step 1. ethyl 2-(4-((tert-butoxycarbonyl)amino)phenyl)thiazole-5-carboxylate was prepared following General experimental procedure 1. (4-((tert-butoxycarbonyl)amino)phenyl)boronic acid (2.0 g, 8.4 mmol) and ethyl 2-bromothiazole-5-carboxylate (2.0 g, 8.4 mmol) gave ethyl 2-(4-((tert-butoxycarbonyl)amino)phenyl)thiazole-5-carboxylate (1.26 g, 3.62 mmol, 43%) as a yellow solid. ¹H NMR (299 MHz, CDCl₃) δ 8.39 (s, 1H), 7.94 (d, J=8.8 Hz, 2H), 7.49 (d, J=8.6 Hz, 2H), 6.66 (s, 1H), 4.40 (q, J=7.1 Hz, 2H), 1.55 (d, J=1.0 Hz, 10H), 1.42 (t, J=7.1 Hz, 3H).

Step 2. 2-(4-((tert-butoxycarbonyl)amino)phenyl)thiazole-5-carboxylic acid was prepared following General experimental procedure 2. Ethyl 2-(4-((tert-butoxycarbonyl)amino)phenyl)thiazole-5-carboxylate (1.2 g, 3.4 mmol) gave 2-(4-((tert-butoxycarbonyl)amino)phenyl)thiazole-5-carboxylic acid (1.0 g, 3.1 mmol, 91%) as a white solid.

Step 3. methyl (2-(4-((tert-butoxycarbonyl)amino)phenyl)thiazole-5-carbonyl)-L-serinate was prepared following General experimental procedure 4. 2-(4-((tert-butoxycarbonyl)amino)phenyl)thiazole-5-carboxylic acid (1.6 g, 5.0 mmol) and H-Ser-OMe.HCl (0.93 g, 1.2 eq., 6.0 mmol) gave methyl (2-(4-((tert-butoxycarbonyl)amino)phenyl)thiazole-5-carbonyl)-L-serinate (1.4 g, 3.3 mmol, 67%) as an orange oil. LCMS (General 3 acidic) RT: 0.99 min; area % (254 nm): 88%; m/z=422.3 [M+H]⁺.

Step 4. (2-(4-((tert-butoxycarbonyl)amino)phenyl)thiazole-5-carbonyl)-L-serine was prepared following General experimental procedure 2. Methyl (2-(4-((tert-butoxycarbonyl)amino)phenyl)thiazole-5-carbonyl)-L-serinate (1.4 g, 3.3 mmol) gave (2-(4-((tert-butoxycarbonyl)amino)phenyl)thiazole-5-carbonyl)-L-serine (0.98 g, 2.4 mmol, 72%).

Step 5. Methyl N-((2-(4-((tert-butoxycarbonyl)amino)phenyl)thiazole-5-carbonyl)-L-seryl)-O-(tert-butyldiphenylsilyl)-L-serinate was prepared following General experimental procedure 4. (2-(4-((tert-butoxycarbonyl)amino)phenyl)thiazole-5-carbonyl)-L-serine (0.98 g, 2.4 mmol) and methyl O-(tert-butyldiphenylsilyl)-L-serinate hydrochloride (1.1 g, 1.2 eq. 2.9 mmol) gave crude methyl N-((2-(4-((tert-butoxycarbonyl)amino)phenyl)thiazole-5-carbonyl)-L-seryl)-O-(tert-butyldiphenylsilyl)-L-serinate (2.25 g) which was used as such in the next step

Step 6. Methyl N—(O-acetyl-N-(2-(4-((tert-butoxycarbonyl)amino)phenyl)thiazole-5-carbonyl)-L-seryl)-O-(tert-butyldiphenylsilyl)-L-serinate was prepared following General experimental procedure 5. Crude methyl N-((2-(4-((tert-butoxycarbonyl)amino)phenyl)thiazole-5-carbonyl)-L-seryl)-O-(tert-butyldiphenylsilyl)-L-serinate (2.25 g) and acetic anhydride (0.34 mL, 3.6 mmol) gave methyl N—(O-acetyl-N-(2-(4-((tert-butoxycarbonyl)amino)phenyl)thiazole-5-carbonyl)-L-seryl)-O-(tert-butyldiphenylsilyl)-L-serinate (0.40 g, 0.51 mmol, 21% ver 2 steps). LCMS (General 3 acidic) RT: 1.78 min; area % (214 nm): 100%; m z=789.6 [M+H]⁺.

Step 7. Methyl 2-(2-(2-(4-((tert-butoxycarbonyl)amino)phenyl)thiazole-5-carboxamido)acrylamido)acrylate was prepared following General experimental procedure 7. Methyl N—(O-acetyl-N-(2-(4-((tert-butoxycarbonyl)amino)phenyl)thiazole-5-carbonyl)-L-seryl)-O-(tert-butyldiphenylsilyl)-L-serinate (0.19 g, 0.24 mmol) gave methyl 2-(2-(2-(4-((tert-butoxycarbonyl)amino)phenyl)thiazole-5-carboxamido)acrylamido)acrylate (7.8 mg, 16 μmol, 27%) as a white solid LC-MS (22010199A TFA LCMS-5 Cl): RT: 1.353 min; Area 97.7% (215 nm), 96.7% (304 nm); m z=473.2 [M+H]⁺. ¹H NMR (400 MHz, CDCl₃) δ (ppm) 8.66 (s, 1H), 8.57 (s, 1H), 8.24 (s, 1H), 7.92 (d, J=8.8 Hz, 2H), 7.48 (d, J=8.8 Hz, 2H), 6.72 (d, J=2.5 Hz, 1H), 6.67 (s, 1H), 6.63 (s, 1H), 6.04 (d, J=1.0 Hz, 1H), 5.49 (dd, J=2.3 Hz, J=1.3 Hz, 1H), 3.91 (s, 1H), 1.54 (s, 9H).

### Example 6: Compound 6

### Methyl 2-(2-(2-(4-acetamidopheny 1)thiazole-4-carboxamido)acrylamido)acrylate

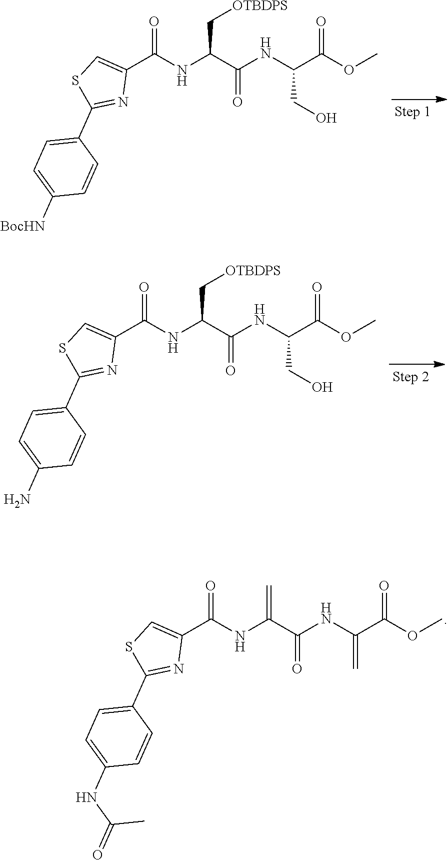

Step 1. Methyl N-(2-(4-aminophenyl)thiazole-4-carbonyl)-O-(tert-butyldiphenylsilyl)-L-seryl-L-serinate was prepared using the following procedure. Methyl N-(2-(4-((tert-butoxycarbonyl)amino)phenyl)thiazole-4-carbonyl)-O-(tert-butyldiphenylsilyl)-L-seryl-L-serinate (as prepared in step 3) (0.80 g, 1 eq., 1.1 mmol) was dissolved in CH₂C₃ (5 mL) and TFA (5 mL) was added. The resulting mixture was stirred at room temperature for 1 hour. The mixture was diluted with CH₂C₂ (50 mL) and poured into 1M NaOH (aq. 150 mL). The layers were separated, and the aqueous phase was extracted with CH₂Cl₂ (50 mL). The combined organic layers were washed with brine, dried over Na₂SO₄, filtered, and concentrated to provide methyl N-(2-(4-aminophenyl)thiazole-4-carbonyl)-O-(tert-butyldiphenylsilyl)-L-seryl-L-serinate (0.65 g, 1.0 mmol, 94%) as yellow oil.

Step 2. Methyl (S)-2-(2-(2-(4-acetamidophenyl)thiazole-4-carboxamido)-3-((methylsulfonyl)oxy)propanamido)acrylate was prepared using the following procedure. Methyl N-(2-(4-aminophenyl)thiazole-4-carbonyl)-O-(tert-butyldi phenylsilyl)-L-seryl-L-serinate (0.22 g, 1 eq., 0.34 mmol) dissolved in CH₂Cl₂ (5 mL) and DIPEA (0.18 mL, 3 eq., 1.0 mmol) and acetic acid (41 mg, 39 μL, 2 Eq, 0.68 mmol) were added. Then, COMU (0.17 g, 1.2 eq., 0.41 mmol) was added and the resulting mixture was stirred for 16 hours at room temperature. LCMS monitoring showed partial conversion to the bis acetylated product. Additional DiPEA (0.18 mL, 3 Eq, 1.0 mmol), acetic acid (39 μL, 2 eq., 0.68 mmol) and COMU (0.17 g, 1.2 eq., 0.41 mmol) were added and the mixture was stirred for 24 hours. The mixture was diluted with CH₂Cl₂ (20 mL) and washed with HCl (1M, 20 mL) NaHCO₃(aq. sat. 20 mL) and brine, dried over Na₂SO₄, filtered, and concentrated. The residue was dissolved in CH₂Cl₂ (5 mL) and DBU (0.15 mL, 3 eq., 1.0 mmol) was added. The resulting mixture was stirred for 1 hour. A solution of tetrabutylammonium fluoride in THE (0.51 mL, 1.0 M, 1.5 eq., 0.51 mmol) was added and the resulting mixture was stirred for 1 hour. The mixture was diluted with CH₂C₂ (20 mL) and washed with HCl (1M, 20 mL) and brine, dried over Na₂SO₄, filtered, and concentrated. The residue was dissolved in CH₂Cl₂(5 mL) and methane sulfonyl chloride (40 μL, 1.5 eq., 0.51 mmol) and triethylamine (0.24 mL, 5 eq., 1.7 mmol) were added. The resulting mixture was stirred for 90 minutes at room temperature after which full mesylation and partial elimination were observed by LCMS monitoring. The mixture was diluted with CH₂C₂ (20 mL) and washed with HCl (1M, 20 mL) and brine, dried over Na₂SO₄, filtered, and concentrated. The residue was dissolved in CH₂Cl₂ (5 mL) and DBU (0.16 g, 0.15 mL, 3 Eq, 1.0 mmol) was added. The resulting mixture was stirred for 90 minutes at room temperature. The mixture was diluted with CH₂C₂ (20 mL) and washed with HCl (1M, 20 mL) and brine, dried over Na₂SO₄, filtered, and concentrated. The crude material was purified by automated reverse phase FCC to provide methyl 2-(2-(2-(4-acetamidophenyl)thiazole-4-carboxamido)acrylamido)acrylate (5.2 mg, 0.34 mmol, 3.7%) as white solid. LCMS (22010199C TFA LCMS-5 C₃) RT: 1.062 min; area % (215 nm): 91.0%; m z=415.0 [M+H]⁺. ¹H NMR (400 MHz, CDCl₃) δ 9.99 (s, 1H), 8.53 (s, 1H), 8.10 (s, 1H), 8.00-7.92 (m, 2H), 7.61 (d, J=8.3 Hz, 2H), 7.29 (s, 1H), 6.76 (d, J=2.2 Hz, 1H), 6.69 (s, 1H), 6.01 (d, J=1.4 Hz, 1H), 5.48 (t, J=1.9 Hz, 1H), 3.88 (s, 3H), 2.21 (s, 3H).

### Example 7: Compound 16

### Methyl 2-(2-(2-phenylthiazole-4-carboxamido)acrylamido)acrylate

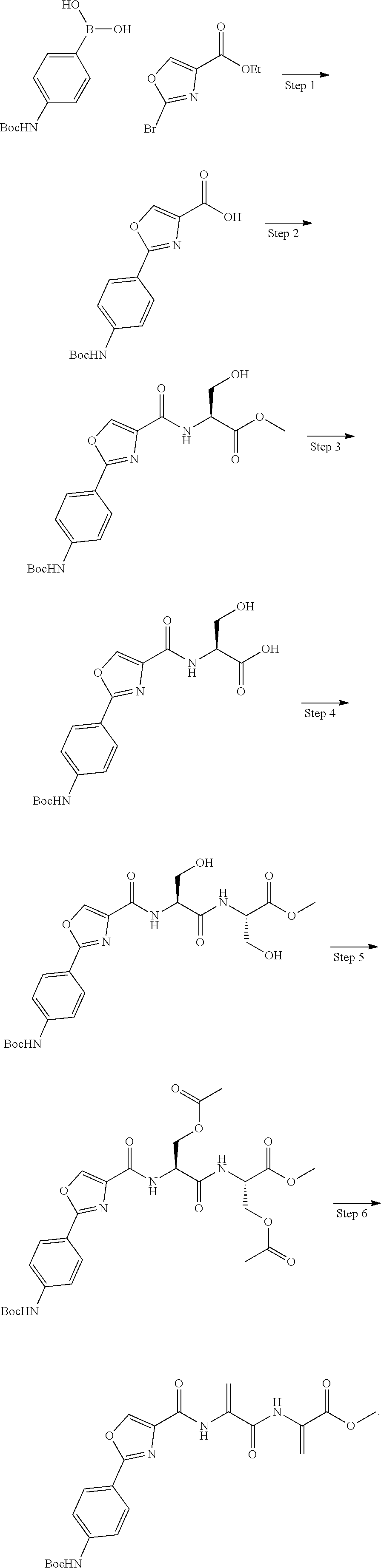

Step 1. 2-(4-((tert-butoxycarbonyl)amino)phenyl)oxazole-4-carboxylic acid was prepared using the following procedure. To a solution of (4-((tert-butoxycarbonyl)amino)phenyl)boronic acid (1.0 g, 4.2 mmol) and ethyl 2-bromooxazole-4-carboxylate (0.93 g, 4.2 mmol) in 1,2-Dimethoxyethane (25 mL) were added sodium carbonate (aq., 2 M, 21 mL, 10 eq., 42 mmol) and palladium tetrakis (0.24 g, 0.05 eq., 0.21 mmol). The resulting mixture was stirred at 80° C. for 16 hours. The mixture was cooled to room temperature and diluted with EtOAc. The mixture was washed with water. The aqueous layer was acidified by addition of 1 M HCl. The formed precipitate was isolated by filtration and dried under reduced pressure to provide 2-(4-((tert-butoxycarbonyl)amino)phenyl)oxazole-4-carboxylic acid (0.66 g, 2.2 mmol, 51%) as a white solid. ¹H NMR (299 MHz, CD30D) 8 8.52 (s, 1H), 8.00 (m, 2H), 7.60 (m, 2H), 1.55 (s, 9H).

Step 2. Methyl (2-(4-((tert-butoxycarbonyl)amino)phenyl)oxazole-4-carbonyl)-L-serinate was prepared following General experimental procedure 4. 2-(4-((tert-butoxycarbonyl)amino)phenyl)oxazole-4-carboxylic acid (0.66 g, 2.2 mmol) and methyl L-serinate hydrochloride (0.41 g, 1.2 eq., 2.6 mmol) gave methyl (2-(4-((tert-butoxycarbonyl)amino)phenyl)oxazole-4-carbonyl)-L-serinate (0.98 g, 2.4 mmol, quant.) as an orange oil.

Step 3. (2-(4-((tert-butoxycarbonyl)amino)phenyl)oxazole-4-carbonyl)-L-serine was prepared following General experimental procedure 2. Methyl (2-(4-((tert-butoxycarbonyl)amino)phenyl)oxazole-4-carbonyl)-L-serinate (0.98 g, 2.4 mmol) gave (2-(4-((tert-butoxycarbonyl)amino)phenyl)oxazole-4-carbonyl)-L-serine (0.64 g, 1.6 mmol, 68%) as an off-white solid. ¹H NMR (299 MHz, cd₃od) 6 8.43 (s, 1H), 8.05-7.96 (m, 2H), 7.60 (m, 2H), 4.71 m, 1H), 4.14-3.94 (m, 2H), 3.79-3.69 (m, 1H), 1.55 (s, 9H).

Step 4. Methyl (2-(4-((tert-butoxycarbonyl)amino)phenyl)oxazole-4-carbonyl)-L-seryl-L-serinate was prepared following General experimental procedure 4. (2-(4-((tert-butoxycarbonyl)amino)phenyl)oxazole-4-carbonyl)-L-serine (0.20 g, 0.51 mmol) and methyl L-serinate hydrochloride (95 mg, 1.2 eq., 0.61 mmol) gave methyl (2-(4-((tert-butoxycarbonyl)amino)phenyl)oxazole-4-carbonyl)-L-seryl-L-serinate (0.18 g, 0.37 mmol, 72%) as a colorless oil.

Step 5. Methyl O-acetyl-N—(O-acetyl-N-(2-(4-((tert-butoxycarbonyl)amino)phenyl)oxazole-4-carbonyl)-L-seryl)-L-serinate was prepared following General experimental procedure 5. Methyl (2-(4-((tert-butoxycarbonyl)amino)phenyl)oxazole-4-carbonyl)-L-seryl-L-serinate (0.18 g, 0.37 mmol) and acetic anhydride (90 μL, 2.6 eq., 0.95 mmol) gave methyl O-acetyl-N—(O-acetyl-N-(2-(4-((tert-butoxycarbonyl)amino)phenyl)oxazole-4-carbonyl)-L-seryl)-L-serinate (0.12 g, 0.20 mmol, 56%). ¹H NMR (299 MHz, CDCb) 6 8.26 (s, 1H), 7.98 (d, J=8.6 Hz, 2H), 7.79 (d, J=8.0 Hz, 1H), 7.51 (d, J=8.6 Hz, 2H), 7.32 (d, J=7.8 Hz, 1H)), 6.88 (s, 1H), 5.04 (m, 1H), 4.87 (m, 1H), 4.58-4.36 (m, 4H), 3.80 (m, 3H), 2.15-1.98 (m, 6H), 1.54 (s, 9H).

Step 6. Methyl (2-(2-(4-((tert-butoxycarbonyl)amino)phenyl)oxazole-4-carboxamido)acryloyl)-L-serinate was prepared following General experimental procedure 8. Methyl O-acetyl-N—(O-acetyl-N-(2-(4-((tert-butoxycarbonyl)amino)phenyl)oxazole-4-carbonyl)-L-seryl)-L-serinate (0.12 g, 0.20 mmol) gave methyl (2-(2-(4-((tert-butoxycarbonyl)amino)phenyl)oxazole-4-carboxamido)acryloyl)-L-serinate (29 mg, 64 μmol, 32%) as a white solid. LC-MS (22010199A TFA LCMS-5 Cl): rt 1.560 min; Area 99.7% (215 nm), 99.2% (304 nm); m z=[M+H]⁺=457.2 ¹H NMR (400 MHz, CDCl₃) δ (ppm) 9.57 (s, 1H), 8.54 (s, 1H), 8.25 (s, 1H), 8.02 (d, J=8.7 Hz, 2H), 7.49 (d, J=8.7 Hz, 2H), 6.75 (d, J=2.2 Hz), 6.71 (s, 1H), 6.64 (s, 1H), 6.03 (d, J=1.0 Hz, 1H), 5.49 (t, J=1.9 Hz, 1H), 3.90 (s, 3H), (s, 9H).

### Example 8: Compound 49

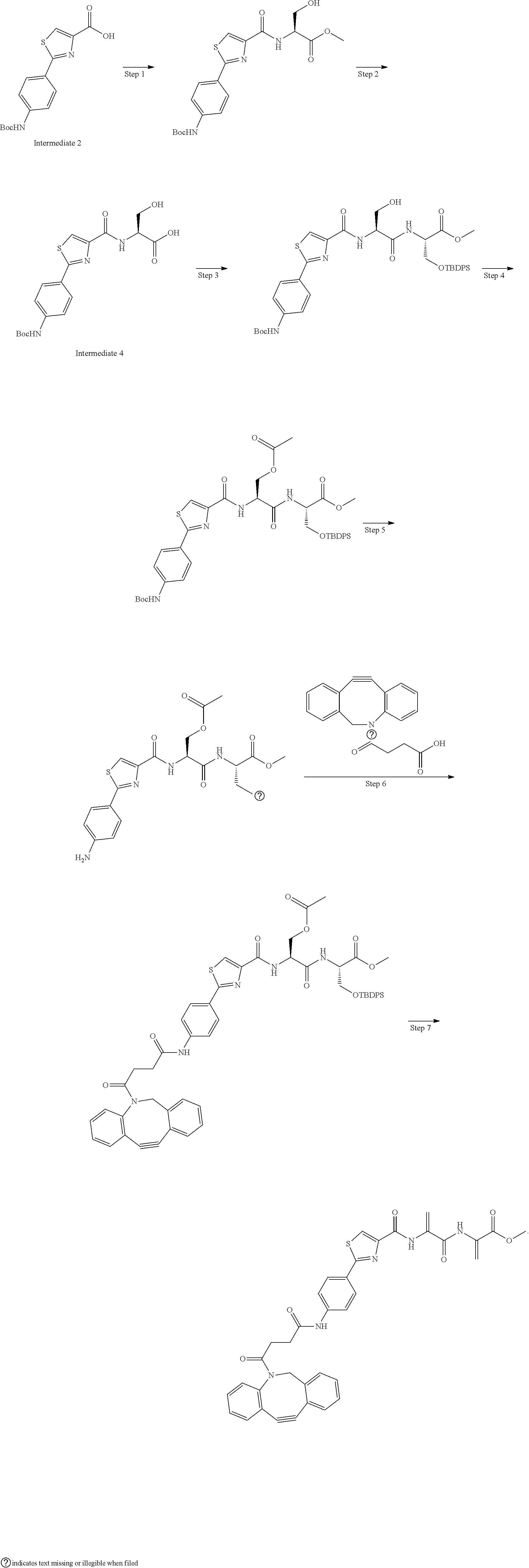

Step. 1 Methyl (2-(4-((tert-butoxycarbonyl)amino)phenyl)thiazole-4-carbonyl)-L-serinate was prepared following General experimental procedure 4. 2-(4-((tert-butoxycarbonyl)amino)phenyl)thiazole-4-carboxylic acid (Intermediate 2) (0.68 g, 2.1 mmol) and methyl L-serinate hydrochloride (0.40 g, 1.2 eq., 2.6 mmol) gave methyl (2-(4-((tert-butoxycarbonyl)amino)phenyl)thiazole-4-carbonyl)serinate (0.87 g, 2.1 mmol, 97%) as an off-white solid. LCMS (General 3 basic) RT: 1.44 min; area % (254 nm): 98%; m z=420.3 [M−H]⁻.

Step 2. (2-(4-((tert-butoxycarbonyl)amino)phenyl)thiazole-4-carbonyl)-L-serine (Intermediate 4) was prepared following General experimental procedure 2. Methyl (2-(4-((tert-butoxycarbonyl)amino)phenyl)thiazole-4-carbonyl)-L-serinate (0.88 g, 2.1 mmol) gave (2-4-((tert-butoxycarbonyl)amino)phenyl)thiazole-4-carbonyl)-L-serine (Intermediate 4) (0.84 g, 2.1 mmol, quant) as a white solid.

Step 3. Methyl N-((2-(4-((tert-butoxycarbonyl)amino)phenyl)thiazole-4-carbonyl)-L-seryl)-O-(tert-butyldiphenylsilyl)-L-serinate was prepared following General experimental procedure 4. (2-(4-((tert-butoxycarbonyl)amino)phenyl)thiazole-4-carbonyl)-L-serine (Intermediate 4) (0.87 g, 2.1 mmol) and L-Ser(OTBDPS)—OMe.HCl (1.0 g, 1.2 eq., 2.6 mmol) gave methyl N-((2-(4-((tert-butoxycarbonyl)amino)phenyl)thiazole-4-carbonyl)-L-seryl)-O-(tert-butyldiphenylsilyl)-L-serinate (1.5 g, 1.9 mmol, 91%) LCMS (General 3 acidic) RT: 1.75 min; area % (254 nm): 98%; m z=747.6 [M+H]⁺.

Step 4. Methyl N—(O-acetyl-N-(2-(4-((tert-butoxycarbonyl)amino)phenyl)thiazole-4-carbonyl)-L-seryl)-O-(tert-butyldiphenylsilyl)-L-serinate was prepared following General experimental procedure 5. Methyl N-((2-(4-((tert-butoxycarbonyl)amino)phenyl)thiazole-4-carbonyl)-L-seryl)-O-(tert-butyldiphenylsilyl)-L-serinate (1.5 g, 1.9 mmol) gave methyl N—(O-acetyl-N-(2-(4-((tert-butoxycarbonyl)amino)phenyl)thiazole-4-carbonyl)-L-seryl)-O-(tert-butyldiphenylsilyl)-L-serinate (1.6 g, 2.0 mmol, quant) as a yellow foam. LCMS (General 3 acidic) RT: 1.84 min; area % (254 nm): 98%; m z=789.6 [M+H]⁺.

Step 5. Methyl N—(O-acetyl-N-(2-(4-aminophenyl)thiazole-4-carbonyl)-L-seryl)-O-(tert-butyldiphenylsilyl)-L-serinate was prepared using the following procedure. To a solution of methyl N—(O-acetyl-N-(2-(4-((tert-butoxycarbonyl)amino)phenyl)thiazole-4-carbonyl)-L-seryl)-O-(tert-butyldiphenylsilyl)-L-serinate (1.6 g, 2.0 mmol) in CH₂Ch (30 mL) was added TFA (4.6 mL, 30 eq. 60 mmol) and the resulting mixture was stirred for 1 hour, the reaction mixture was quenched with saturated aqueous NaHCO₃until gas formation stopped and pH paper showed the mixture pH ˜7-8. The mixture was extracted with CH₂C₂ (3×100 ml) and the combined organics were dried over Na₂SO₄, filtered and concentrated to provide methyl N—(O-acetyl-N-(2-(4-aminophenyl)thiazole-4-carbonyl)-L-seryl)-O-(tert-butyldiphenylsilyl)-L-serinate (1.3 g, 1.8 mmol, 91%) LCMS (General 3 basic) RT: 2.01 min; area % (254 nm): 72%; m z=687.4 [M−H]⁻.

Step 6. The DBCO coupled intermediate was prepared using the following procedure. To a solution of DBCO acid (0.37 g, 1.2 mmol) and triethylamine (0.19 mL, 1.1 eq., 1.3 mmol) in CH₂Ch (6 mL) was added at 0° C. propyl chloroformate (0.15 mL, 1.1 Eq, 1.3 mmol) and the resulting mixture was stirred at 0° C. for 1 hour. A solution of methyl N—(O-acetyl-N-(2-(4-aminophenyl)thiazole-4-carbonyl)-L-seryl)-O-(tert-butyldi phenylsilyl)-L-serinate (0.61 g, 0.73 eq., 0.88 mmol) in CH₂Cl₂ (3 mL) was added and the resulting mixture was stirred for 2 hours at room temperature. The mixture was washed with NaHCO₃ (sat., 3×10 mL) and HCl (1M, 3x 10 mL), dried over Na₂SO₄, filtered and concentrated. The crude material was purified by automated FCC to provide the desired product (0.52 g, 0.54 mmol, 45%) of a slightly yellow foam. LCMS (General 3 acidic) RT: 1.86 min; area % (254 nm): 86%; m z=976.5 [M+H]⁺.

Step 7. DBCO coupled bisdehydroalanine compound was prepared following General experimental procedure 5. DBCO coupled intermediate (0.52 g, 0.54 mmol) gave DBCO coupled bisdehydroalanine compound (24 mg, 37 μmol, 6.9%) as a white solid. LCMS (22010199D TFA LCMS-5 C₈) RT: 1.642 min; area % (215 nm): 79.2%; m z=660.2 [M+H]⁺. ¹H NMR (400 MHz, CDCl₃) δ 10.05 (s, 1H), 8.56 (s, 1H), 8.42 (s, 1H), 8.09 (s, 1H), 7.84 (d, J=8.6 Hz, 2H), 7.71 (d, J=7.4 Hz, 1H), 7.50-7.28 (m, 8H), 7.19 (dd, J=7.4, 1.5 Hz, 1H), 6.78 (d, J=2.2 Hz, 1H), 6.73 (s, 1H), 6.03 (d, J=1.3 Hz, 1H), 5.50 (t, J=1.9 Hz, 1H), 5.19 (d, J=13.9 Hz, 1H), 3.91 (s, 3H), 3.73 (d, J=13.8 Hz, 1H), 2.86 (m, 1H), 2.45 (m, 2H), 2.07 (m, 1H).

### Example 9: Compound 50

### Methyl 2-(2-(2-(4-(hex-5-ynamido)phenyl)thiazole-4-carboxamido)acrylamido)acrylate

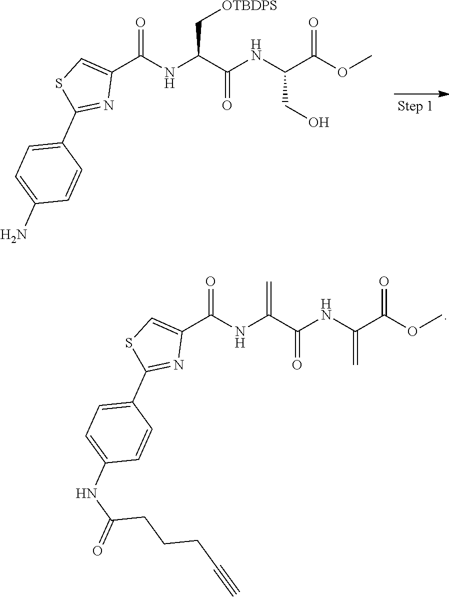

Step 1. Methyl 2-(2-(2-(4-(hex-5-ynamido)phenyl)thiazole-4-carboxamido)acrylamido)acrylate was prepared with the following procedure. Methyl N-(2-(4-aminophenyl)thiazole-4-carbonyl)-O-(tert-butyldiphenylsilyl)-L-seryl-L-serinate (as prepared in step 1) (0.22 g, 0.334 mmol) dissolved in CH₂Cl₂ (5 mL) and DiPEA (0.18 mL, 3 eq., 1.0 mmol) and hex-5-ynoic acid (56 μL, 1.5 eq., 0.51 mmol) were added. Then COMU (0.18 g, 1.2 eq., 0.41 mmol) was added and the resulting mixture was stirred for 16 hours at room temperature. LCMS monitoring showed partial conversion to the bis acylated product. Additional DiPEA (0.18 mL, 3 eq., 1.0 mmol), hex-5-ynoic acid (57 mg, 56 μL, 1.5 eq., 0.510 mmol) and COMU (0.18 mg, 1.2 eq., 0.41 mmol) were added and the resulting mixture was stirred for stirred for 24 hours. The mixture was diluted with CH₂C₂ (20 mL) and washed with HCl (1M, 20 mL) NaHCO₃(aq. sat. 20 mL) and brine, dried over Na₂SO₄, filtered, and concentrated. The residue was dissolved in CH₂C₂ (5 mL) and added DBU (0.15 mL, 3 eq., 1.0 mmol) was added. The resulting mixture was stirred for 1 hour. A solution of tetrabutylammonium fluoride in THE (0.51 mL, 1.0 M, 1.5 eq., 0.51 mmol) was added and the resulting mixture was stirred for 1 hour. The mixture was diluted with CH₂Ch₂ (20 mL) and washed with HCl (1M, 20 mL) and brine, dried over Na₂SO₄, filtered, and concentrated. The residue was dissolved in CH₂Cl₂ (5 mL) and methane sulfonyl chloride (40 μL, 1.5 eq., 0.51 mmol) and triethylamine (0.17 g, 0.24 mL, 5 eq., 1.7 mmol) were added. The resulting mixture was stirred for 90 minutes at room temperature after which full mesylation and partial elimination were observed by LCMS monitoring. The mixture was diluted with CH₂C₂ (20 mL) and washed with HCl (1M, 20 mL) and brine, dried over Na₂SO₄, filtered, and concentrated. The residue was dissolved in CH₂C₂ (5 mL) and DBU (0.16 g, 0.15 mL, 3 eq., 1.0 mmol) was added. The resulting mixture was stirred for 90 minutes at room temperature. The mixture was diluted with CH₂C₂ (20 mL) and washed with HCl (1M, 20 mL) and brine, dried over Na₂SO₄, filtered, and concentrated. The crude material was purified by automated reverse phase FCC to provide methyl 2-(2-(2-(4-(hex-5-ynamido)phenyl)thiazole-4-carboxamido)acrylamido)acrylate (4.5 mg, 9.6 μmol, 2.8%) as a white solid. LCMS (22010199C TFA LCMS-5 C₃) RT: 1.287 min; area % (215 nm): 93.0%; m z=467.2 [M+H]⁺. ¹H NMR (400 MHz, CDCl₃) δ 9.98 (s, 1H), 8.53 (s, 1H), 8.00-7.92 (m, 2H), 7.62 (d, J=8.3 Hz, 2H), 7.31 (s, 1H), 6.76 (d, J=2.2 Hz, 1H), 6.69 (s, 1H), 6.01 (d, J=1.2 Hz, 1H), 5.48 (t, J=1.9 Hz, 1H), 3.88 (s, 3H), 2.55 (t,J=7.3 Hz, 2H), 2.34 (td,J=6.7, 2.6 Hz, 2H), 2.04-1.92 (m, 3H).

### Example 10: Compound 51

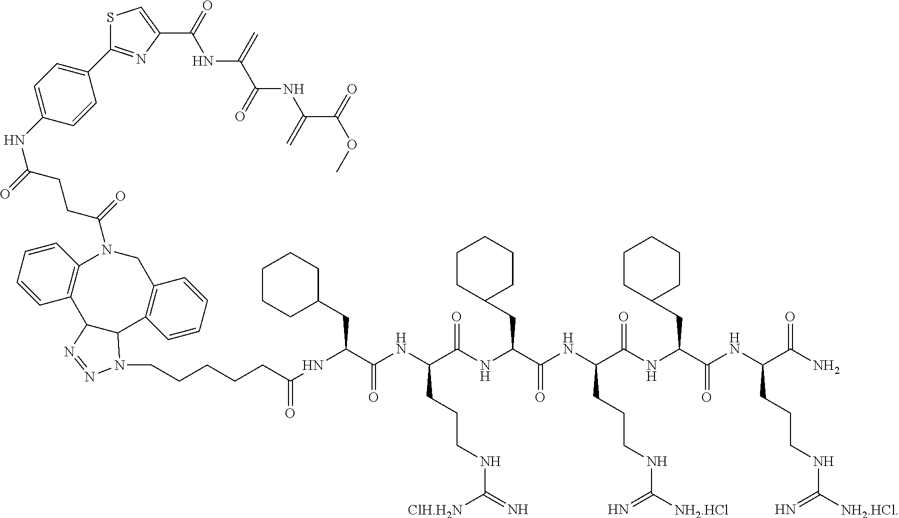

Compound 51 was prepared following General procedure for conjugation chemistry between azido-peptide and. 49 (3.0 mg, 4.5 μmol) and 81 (5.4 mg, 4.5 μmol) gave 51 (3.5 mg, 1.9 μmol, 41%) as a white solid. LCMS (30833 LCMS-6): RT: 2.788 min & 2.822 min (regioisomers); Area: 95.3% (215 nm), 99.6% (EL SD); m/z=1744.1 [M+H]+

### Example 11: Compound 52

### Methyl 2-(2-(2-(4-((tert-butoxycarbonyl)amino)phenyl)-5-methylthiazole-4-carboxamido)acrylamido)acrylate

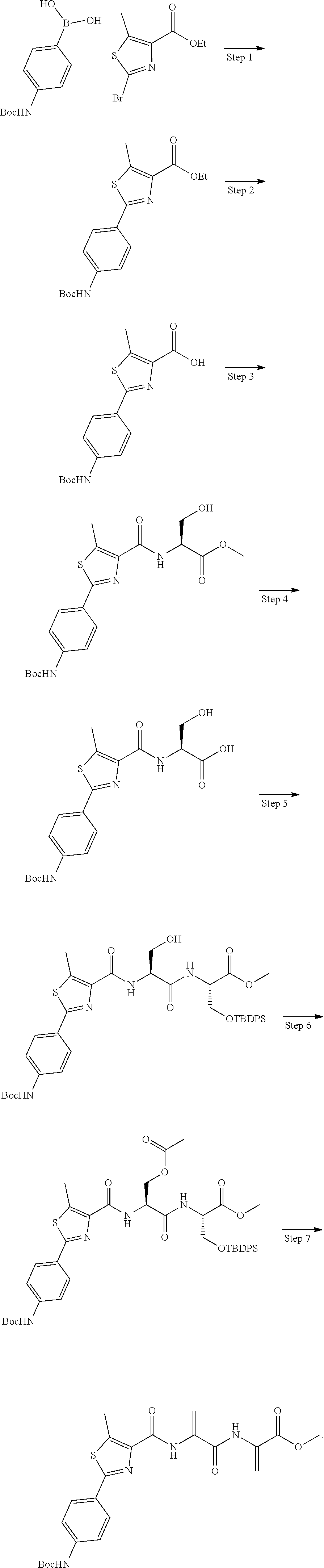

Step 1. Methyl 2-(4-((tert-butoxycarbonyl)amino)phenyl)-5-methylthiazole-4-carboxylate was prepared following General experimental procedure 1. Methyl-2-Bromo-5-methylthiazole-4-carboxylate (0.91 g, 3.8 mmol) and (4-((tertbutoxycarbonyl) amino)phenyl)boronic acid (0.86 g, 3.6 mmol) gave methyl 2-(4-((tert-10 butoxycarbonyl)amino)phenyl)-5-methylthiazole-4-carboxylate (1.1 g, 3.1 mmol 88%). LCMS (General 3 basic) RT: 1.80 min; area % (214 nm): 87%; m z=347 2 [M−H]⁻.

Step 2. 2-(4-((tert-butoxycarbonyl)amino)phenyl)-5-methylthiazole-4-carboxylic acid was prepared following General experimental procedure 2. Methyl 2-(4-((tert-butoxycarbonyl)amino)phenyl)-5-methylthiazole-4-carboxylate (1.1 g, 3.1 mmol) gave methyl 2-(4-((tert-butoxycarbonyl)amino)phenyl)-5-methylthiazole-4-carboxylate (1.0 g, 3.1 mmol, quant). %). LCMS (General 3 basic) RT: 1.17 min; area % (214 nm): 81%; m z=335.4 [M+H]⁺.

Step 3. Methyl (2-(4-((tert-butoxycarbonyl)amino)phenyl)-5-methylthiazole-4-carbonyl)serinate was prepared following General experimental procedure 3. 2-(4-((tert-butoxycarbonyl)amino)phenyl)-5-methylthiazole-4-carboxylic acid (1.1 g, 3.2 mmol) and L-Ser-OMe.HCl (0.60 g, 1.2 eq. 3.9 mmol) gave crude methyl (2-(4-((tert-butoxycarbonyl)amino)phenyl)-5-methylthiazole-4-carbonyl)serinate (2.6 g), which was used without purification in the next step. LCMS (General 3 basic) RT: 1.61 min; area % (214 nm): 69%; m z=436.2 [M+H]⁺.

Step 4. (2-(4-((tert-butoxycarbonyl)amino)phenyl)-5-methylthiazole-4-carbonyl)serine was prepared following General experimental procedure 2. Crude methyl (2-(4-((tert-butoxycarbonyl)amino)phenyl)-5-methylthiazole-4-carbonyl)serinate (2.6 g crude) gave crude (2-(4-((tert-butoxycarbonyl)amino)phenyl)-5-methylthiazole-4-carbonyl)serine (1.8 g), which was used without purification in the next step. LCMS (General 3 basic) RT: 1.06 min; area % (214 nm): 69%; m z=422.4 [M+H]⁺.

Step 5. Methyl N-((2-(4-((tert-butoxycarbonyl)amino)phenyl)-5-methylthiazole-4-carbonyl)seryl)-O-(tert-butyldiphenylsilyl)-L-serinate was prepared following General experimental procedure 4. (2-(4-((tert-butoxycarbonyl)amino)phenyl)-5-methylthiazole-4-carbonyl)serine (1.4 g, crude) and methyl O-(tert-butyldiphenylsilyl)-L-serinate hydrochloride (1.6 g, 4.0 mmol) gave methyl N-((2-(4-((tert-butoxycarbonyl)amino)phenyl)-5-methylthiazole-4-carbonyl)seryl)-O-(tert-butyldiphenylsilyl)-L-serinate (1.2 g, 1.5 mmol) as a clear yellow oil. LCMS (General 3 acidic) RT: 1.85 min; area % (214 nm): 73%; m z=761.6 [M+H]⁺.

Step 6. Methyl N—(O-acetyl-N-(2-(4-((tert-butoxycarbonyl)amino)phenyl)-5-methylthiazole-4-carbonyl)seryl)-O-(tert-butyldiphenylsilyl)-L-serinate was prepared following General experimental procedure 5. Methyl N-((2-(4-((tert-butoxycarbonyl)amino)phenyl)-5-methylthiazole-4-carbonyl)seryl)-O-(tert-butyldiphenylsilyl)-L-serinate (1.2 g, 1.5 mmol) and acetic anhydride (0.16 mL, 1.1 eq., 1.7 mmol) gave methyl N—(O-acetyl-N-(2-(4-((tert-butoxycarbonyl)amino)phenyl)-5-methylthiazole-4-carbonyl)seryl)-O-(tert-butyldiphenylsilyl)-L-serinate (1.1 g, 1.3 mmol, 87%) as a yellow viscous oil. LCMS (General 3 basic) RT: 2.31 min; area % (214 nm): 86%; m z=801.5 [M+H]⁺.

Step 7. Methyl 2-(2-(2-(4-((tert-butoxycarbonyl)amino)phenyl)-5-methylthiazole-4-carboxamido)acrylamido)acrylate was prepared following General experimental procedure 7. Methyl N—(O-acetyl-N-(2-(4-((tert-butoxycarbonyl)amino)phenyl)-5-methylthiazole-4-carbonyl)seryl)-O-(tert-butyldiphenylsilyl)-L-serinate (0.21 g, 0.26 mmol) gave methyl 2-(2-(2-(4-((tert-butoxycarbonyl)amino)phenyl)-5-methylthiazole-4-carboxamido)acrylamido)acrylate (11 mg, 23 μmol, 13%) as a white solid. LCMS (22010199C TFA LCMS-5 C₃) RT: 1.702 min; area: 91.1% (215 nm); m z 487.2 [M+H]⁺. ¹H NMR (400 MHz, CDC3) 6 10.08 (s, 1H), 8.51 (s, 1H), 7.88-7.80 (m, 2H), 7.42 (d, J=8.6 Hz, 2H), 6.72-6.66 (m, 2H), 6.57 (s, 1H), 6.00 (d, J=1.2 Hz, 1H), 5.42 (t, J=1.8 Hz, 1H), 3.88 (s, 3H), 2.85 (s, 3H), 1.52 (s, 9H).

### Example 12: Compound 53

### methyl (Z)-2-(2-(2-(4-((tert-butoxycarbonyl)amino)phenyl)thiazole-4-carboxamido)acrylamido)but-2-enoate

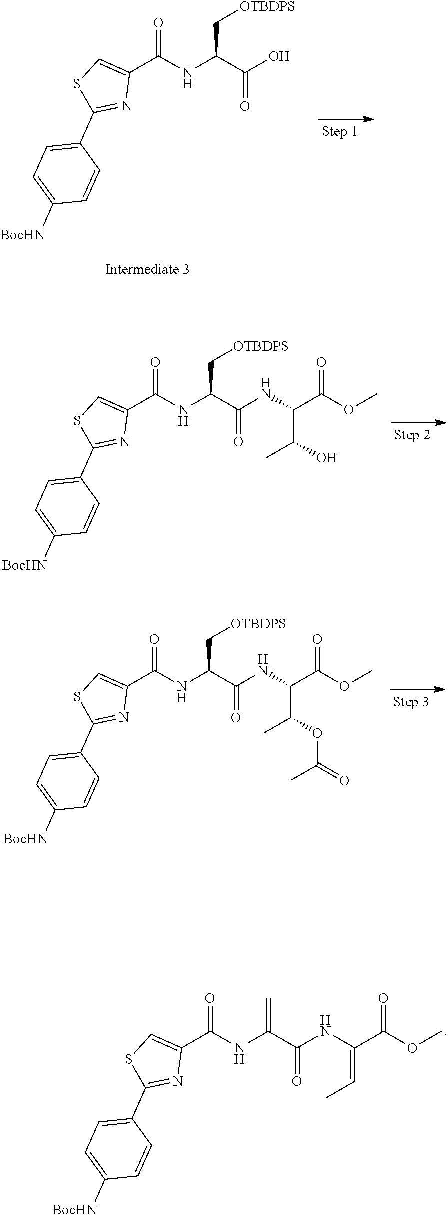

Step 1. Methyl N-(2-(4-((tert-butoxycarbonyl)amino)phenyl)thiazole-4-carbonyl)-O-(tert-butyldiphenylsilyl)seryl-L-threoninate was prepared following General experimental procedure 3. N-(2-(4-((tert-butoxycarbonyl)amino)phenyl)thiazole-4-carbonyl)-O-(tert-butyldiphenylsilyl)serine (Intermediate 3) (0.90 g, 1.4 mmol) and H-Thr-OMe.HCl (0.29 g, 1.2 eq., 1.7 mmol) gave methyl N-(2-(4-((tert-butoxycarbonyl)amino)phenyl)thiazole-4-carbonyl)-O-(tert-butyldiphenylsilyl)seryl-L-threoninate (0.29 g, 0.38 mmol, 27%) as a light yellow foam. LCMS (General 3 basic) RT: 2.20 min; area % (254 nm): 81%; m z=761.3 [M+H]⁺.

Step 2. Methyl O-acetyl-N—(N-(2-(4-((tert-butoxycarbonyl)amino)phenyl)thiazole-4-carbonyl)-O-(tert-butyldiphenylsilyl)seryl)-L-threoninate was prepared following General experimental procedure 5. Methyl N-(2-(4-((tert-butoxycarbonyl)amino)phenyl)thiazole-4-carbonyl)-O-(tert-butyldiphenylsilyl)seryl-L-threoninate (0.29 g, 0.38 mmol) and acetic anhydride (37 μL, 1.05 eq., 0.39 mmol) gave methyl O-acetyl-N—(N-(2-(4-((tert-butoxycarbonyl)amino)phenyl)thiazole-4-carbonyl)-O-(tert-butyldiphenylsilyl)seryl)-L-threoninate (0.30 g, 0.37 mmol, 98%) as a light yellow foam.

Step 3. Methyl (Z)-2-(2-(2-(4-((tert-butoxycarbonyl)amino)phenyl)thiazole-4-carboxamido)acrylamido)but-2-enoate was prepared following General experimental procedure 7. Methyl O-acetyl-N—(N-(2-(4-((tert-butoxycarbonyl)amino)phenyl)thiazole-4-carbonyl)-O-(tert-butyldiphenylsilyl)seryl)-L-threoninate (0.10 g, 0.13 mmol) gave methyl (Z)-2-(2-(2-(4-((tert-butoxycarbonyl)amino)phenyl)thiazole-4-carboxamido)acrylamido)but-2-enoate (22 mg, 45 μmol, 36%). LCMS (22010199C TFA LCMS-5 C₃) RT: 1.424 min; area % (215 nm): 92.9%; m z=487.2 [M+H]⁺. ¹H NMR (400 MHz, CDCl₃) δ 9.97 (s, 1H), 8.07 (s, 1H), 7.94-7.86 (m, 2H), 7.48 (s, 1H), 7.42 (d, J=8.6 Hz, 2H), 6.93 (q, J=7.2 Hz, 1H), 6.76 (d, J=1.8 Hz, 1H), 6.59 (s, 1H), 5.53 (t, J=1.7 Hz, 1H), 3.79 (s, 3H), 1.82 (dd, J=7.2, 0.7 Hz, 3H), 1.51 (s, 9H).

### Example 13: Compound 55

### Methyl (E)-2-(2-(2-(4-((tert-butoxycarbonyl)amino)phenyl)thiazole-4-carboxamido)acrylamido)but-2-enoate

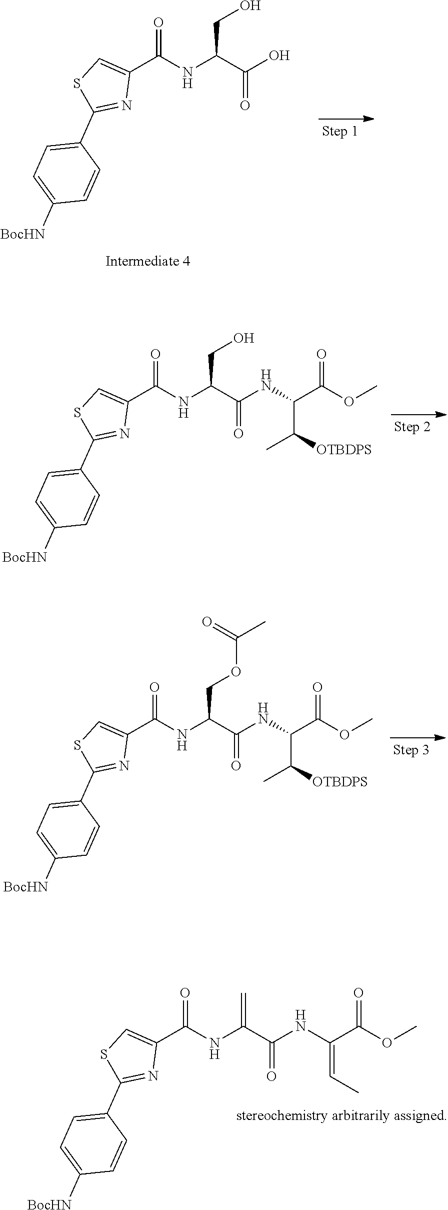

Step 1. Methyl N-((2-(4-((tert-butoxycarbonyl)amino)phenyl)thiazole-4-carbonyl)seryl)-O-(tert-butyldiphenylsilyl)-L-allothreoninate was prepared following General experimental procedure 4. (2-(4-((tert-butoxycarbonyl)amino)phenyl)thiazole-4-carbonyl)serine (Intermediate 4) (785 mg, 1.93 mmol) and methyl O-(tert-butyldiphenylsilyl)-L-allothreoninate (859 mg, 1.2 eq., 2.31 mmol) gave methyl N-((2-(4-((tert-butoxycarbonyl)amino)phenyl)thiazole-4-carbonyl)seryl)-O-(tert-butyldiphenylsilyl)-L-allothreoninate (1.54 g, 2.02 mmol, quant.) as a viscous oil. LCMS (General 3 acidic) RT: 1.81 min; area % (254 nm): 96%; m z=761.3 [M+H]⁺.

Step 2. Methyl N—(O-acetyl-N-(2-(4-((tert-butoxycarbonyl)amino)phenyl)thiazole-4-carbonyl)seryl)-O-(tert-butyldiphenylsilyl)-L-allothreoninate was prepared following General experimental procedure 5. Methyl N-((2-(4-((tert-butoxycarbonyl)amino)phenyl)thiazole-4-carbonyl)seryl)-O-(tert-butyldiphenylsilyl)-L-allothreoninate (1.6 g, 2.1 mmol) and acetic anhydride (0.22 mL, 1.1 eq., 2.3 mmol) gave methyl N—(O-acetyl-N-(2-(4-((tert-butoxycarbonyl)amino)phenyl)thiazole-4-carbonyl)seryl)-O-(tert-butyldiphenylsilyl)-L-allothreoninate (1.37 g, 1.71 mmol, 81%) as a yellow solid. LCMS (General 3 acidic) RT: 1.88 min; area % (254 nm): 75%; m z=803.5 [M+H]⁺.

Step 3. Methyl (E)-2-(2-(2-(4-((tert-butoxycarbonyl)amino)phenyl)thiazole-4-carboxamido)acrylamido)but-2-enoate was prepared following General experimental procedure 7. Methyl N—(O-acetyl-N-(2-(4-((tert-butoxycarbonyl)amino)phenyl)thiazole-4-carbonyl)seryl)-O-(tert-butyldiphenylsilyl)-L-allothreoninate (306 mg, 0.38 mmol) gave methyl methyl (E)-2-(2-(2-(4-((tert-butoxycarbonyl)amino)phenyl)thiazole-4-carboxamido)acrylamido)but-2-enoate (4.6 mg, 9.5 μmol, 2.5%) %). LCMS (22010199C TFA LCMS-5 C₃) RT: 1.470 min; area: 93.8% (215 nm); m/z 487.2 [M+H]⁺. ¹H NMR (400 MHz, CDCl₃) δ 9.98 (s, 1H), 8.17 (s, 1H), 8.06 (s, 1H), 7.95-7.88 (m, 2H), 7.44 (d, J=8.6 Hz, 2H), 7.33 (q, J=7.7 Hz, 2H), 6.70 (d, J=2.0 Hz, 1H), 6.59 (s, 1H), 5.42 (s, 1H), 3.88 (s, 3H), 2.15 (d, J=7.7 Hz, 3H), 1.52 (s, 9H).

### Example 14: Compound 56

### Methyl 2-(2-(2-(4-((tert-butoxycarbonyl)amino)phenyl)thiazole-4-carboxamido)acetamido)acrylate

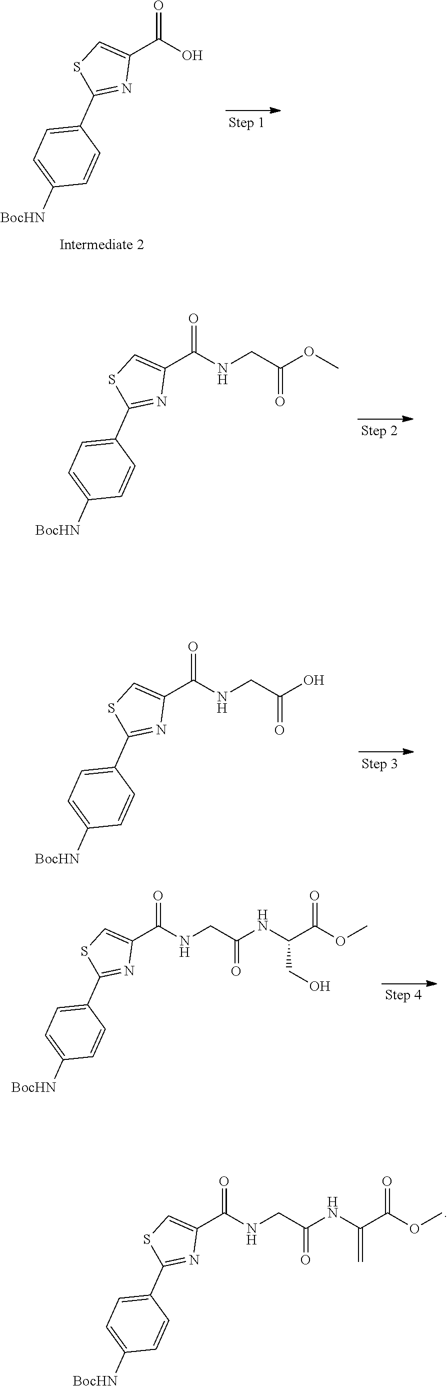 [SMILES: CC(C)(C)OC(=O)Nc1ccc(-c2nc(C(=O)O)cs2)cc1 ; COC(=O)CNC(=O)c1csc(-c2ccc(NC(=O)OC(C)(C)C)cc2)n1 ; CC(C)(C)OC(=O)Nc1ccc(-c2nc(C(=O)NCC(=O)O)cs2)cc1 ; COC(=O)[C@H](CO)NC(=O)CNC(=O)c1csc(-c2ccc(NC(=O)OC(C)(C)C)cc2)n1 ; C=C(NC(=O)CNC(=O)c1csc(-c2ccc(NC(=O)OC(C)(C)C)cc2)n1)C(=O)OC]

Step 1. Methyl (2-(4-((tert-butoxycarbonyl)amino)phenyl)thiazole-4-carbonyl)glycinate was prepared following General experimental procedure 3. 2-(4-((tert-butoxycarbonyl)amino)phenyl)thiazole-4-carboxylic acid (Intermediate 2) (0.41 g, 1 eq., 1.3 mmol) and methyl glycinate hydrochloride (0.17 g, 1 eq., 1.3 mmol) gave methyl (2-(4-((tert-butoxycarbonyl)amino)phenyl)thiazole-4-carbonyl)glycinate (0.37 g, 0.94 mmol, 74%) as a white foam. LCMS (General 3 basic) RT: 1.63 min; area % (254 nm): 92%; m/z=392.2 [M+H]⁺. ¹H NMR (400 MHz, CDCl₃) δ 8.03 (s, 1H), 7.91-7.82 (m, 3H), 7.49-7.41 (m, 2H), 6.71 (s, 1H), 4.26 (d, J=5.7 Hz, 2H), 3.78 (s, 3H), 1.51 (s, 9H).

Step 2. (2-(4-((tert-butoxycarbonyl)amino)phenyl)thiazole-4-carbonyl)glycine was prepared following General experimental procedure 2. Methyl (2-(4-((tert-butoxycarbonyl)amino)phenyl)thiazole-4-carbonyl)glycinate (0.37 mg, 1 eq., 0.94 mmol) gave (2-(4-((tert-butoxycarbonyl)amino)phenyl)thiazole-4-carbonyl)glycine (0.32 g, 0.85 mmol, 90%) as a white solid. LCMS (General 3 basic) RT: 1.12 min; area % (254 nm): 96%; m z=378.2 [M+H]⁺. ¹H NMR (400 MHz, DMSO) δ 9.69 (s, 1H), 8.71 (t, J=6.1 Hz, 1H), 8.24 (s, 1H), 7.98-7.90 (m, 2H), 7.65-7.57 (m, 2H), 3.96 (d, J=6.1 Hz, 2H), 1.49 (s, 9H).

Step 3. Methyl (2-(4-((tert-butoxycarbonyl)amino)phenyl)thiazole-4-carbonyl)glycyl-L-serinate was prepared following General experimental procedure 3. (2-(4-((tert-butoxycarbonyl)amino)phenyl)thiazole-4-carbonyl)glycine (0.31 g, 1 eq, 0.83 mol) and methyl L-serinate hydrochloride (0.16 g, 1.2 eq. 1.0 mmol) gave methyl (2-(4-((tert-butoxycarbonyl)amino)phenyl)thiazole-4-carbonyl)glycylserinate (0.29 mg, 0.60 mmol, 72%). LCMS (General 3 basic) RT: 1.47 min; area % (254 nm): 93%; m/z=479.3 [M+H]⁺. ¹H NMR (400 MHz, MeOD) δ 8.07 (s, 1H), 7.90-7.82 (m, 2H), 7.52-7.44 (m, 2H), 4.56 (t, J=4.3 Hz, 1H), 4.15 (d, J=2.1 Hz, 2H), 3.92-3.76 (m, 2H), 3.71 (s, 3H), 1.49 (s, 9H).

Step 4. Methyl 2-(2-(2-(4-((tert-butoxycarbonyl)amino)phenyl)thiazole-4-carboxamido)acetamido)acrylate was prepared using the following procedure. To a solution of methyl (2-(4-((tert-butoxycarbonyl)amino)phenyl)thiazole-4-carbonyl)glycylserinate (0.25 g, 1 eq., 0.51 mmol) in MeCN (30 mL) was added triethylamine (0.14 mL, 2 eq., 1.0 mmol) and the resulting mixture was stirred for 6 days at room temperature. The mixture was concentrated and purified by automated FCC and automated reverse phase FCC to provide methyl 2-(2-(2-(4-((tert-butoxycarbonyl)amino)phenyl)thiazole-4-carboxamido)acetamido)acrylate (28 mg, 61 μmol, 12%) as white solid. LCMS (22010199D TFA LCMS-5 C₈) RT: 1.382 min; area % (215 nm): 98.0%; m z=461.2 [M+H]⁺. ¹H NMR (400 MHz, CDCl₃) δ 8.36 (s, 1H), 8.09 (d, J=2.4 Hz, 1H), 8.00 (d, J=6.5 Hz, 1H), 7.91-7.80 (m, 2H), 7.45 (d, J=8.3 Hz, 2H), 6.61 (d, J=2.5 Hz, 2H), 5.92 (d, J=1.5 Hz, 1H), 4.24 (d, J=5.9 Hz, 2H), 3.82 (s, 3H), 1.52 (s, 9H).

### Example 15: Compound 57

### Methyl (S)-2-(2-(2-(4-((tert-butoxycarbonyl)amino)phenyl)thiazole-4-carboxamido)propanamido)acrylate

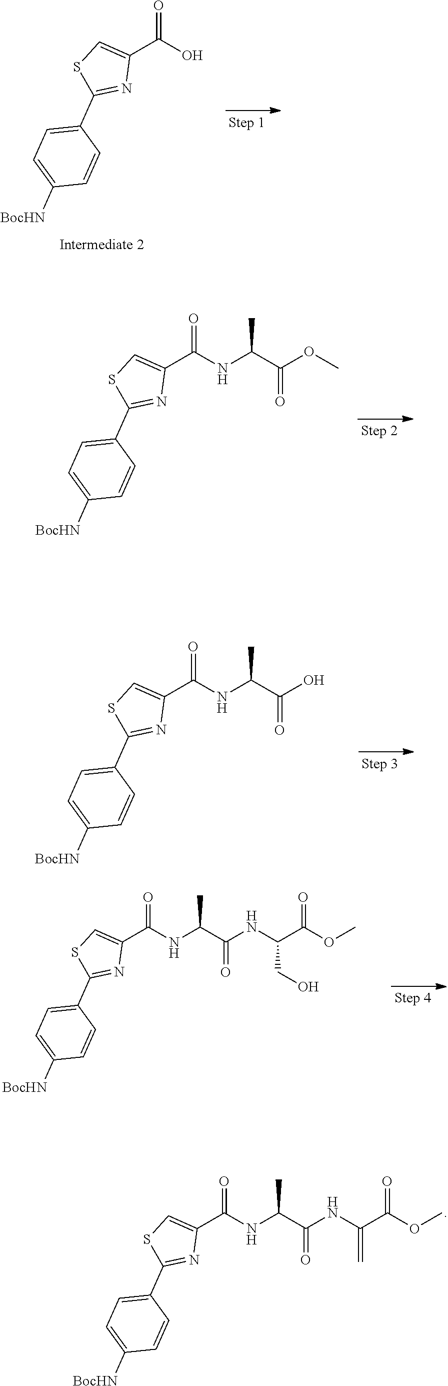 [SMILES: CC(C)(C)OC(=O)Nc1ccc(-c2nc(C(=O)O)cs2)cc1 ; COC(=O)[C@H](C)NC(=O)c1csc(-c2ccc(NC(=O)OC(C)(C)C)cc2)n1 ; C[C@H](NC(=O)c1csc(-c2ccc(NC(=O)OC(C)(C)C)cc2)n1)C(=O)O ; COC(=O)[C@H](CO)NC(=O)[C@H](C)NC(=O)c1csc(-c2ccc(NC(=O)OC(C)(C)C)cc2)n1 ; C=C(NC(=O)[C@H](C)NC(=O)c1csc(-c2ccc(NC(=O)OC(C)(C)C)cc2)n1)C(=O)OC]

Step 1. Methyl (2-(4-((tert-butoxycarbonyl)amino)phenyl)thiazole-4-carbonyl)-L-alaninate was prepared following General experimental procedure 3. 2-(4-((tert-butoxycarbonyl)amino) phenyl)thiazole-4-carboxylic acid (Intermediate 2) (0.53 g, 1.7 mmol) and H-Ala-OMe.HCl (0.27 g, 1.2 eq., 1.9 mmol) gave methyl (2-(4-((tert-butoxycarbonyl)amino)phenyl)thiazole-4-carbonyl)-L-alaninate (0.43 mg, 1.1 mmol, 630%) LCMS (General 3 basic) RT: 1.77 min; area % (254 nm): 86% ; m z=404.2 [M+H].

Step 2. (2-(4-((tert-butoxycarbonyl)amino)phenyl)thiazole-4-carbonyl)-D-alanine was prepared following General experimental procedure 2. Methyl (2-(4-((tert-butoxycarbonyl)amino) phenyl)thiazole-4-carbonyl)-D-alaninate (0.43 g, 1.1 mmol) gave (2-(4-((tert-butoxycarbonyl)amino)phenyl)thiazole-4-carbonyl)-D-alanine (0.41 g, 1.1 mmol, quant.) as a yellow foam. LCMS (General 3 basic) RT: 1.16 min; area % (254 nm): 950; m z=392.2 [M+H]⁺.

Step 3. Methyl (2-(4-((tert-butoxycarbonyl)amino)phenyl)thiazole-4-carbonyl)-L-alanyl-L-serinate was prepared following General experimental procedure 3. (2-(4-((tert-butoxycarbonyl)amino)phenyl)thiazole-4-carbonyl)-D-alanine (0.41 g, 1.1 mmol) and H-Ser-OMe.HCl (0.25 mg, 1.2 eq., 1.38 mmol) gave methyl (2-(4-((tert-butoxycarbonyl)amino)phenyl)thiazole-4-carbonyl)-L-alanyl-L-serinate (0.24 mg, 0.50 mmol 470) as a yellow foam. LCMS (General 3 basic) RT: 1.54 min; area % (254 nm): 85%0; mz=493.2 [M+H]⁺.

Step 4. Methyl (S)-2-(2-(2-(4-((tert-butoxycarbonyl)amino)phenyl)thiazole-4-carboxamido)propanamido)acrylate was prepared using the following procedure. To a solution of methyl (2-(4-((tert-butoxycarbonyl)amino)phenyl)thiazole-4-carbonyl)-L-alanylserinate (0.24 mg, 1 eq., 0.50 mmol) in MeCN (30 mL) was added triethylamine (0.14 mL, 2 eq., 1.0 mmol) and the resulting mixture was stirred for 6 days at room temperature. The mixture was concentrated and purified by automated FCC and automated reverse phase FCC to provide methyl (S)-2-(2-(2-(4-((tert-butoxycarbonyl)amino)phenyl)thiazole-4-carboxamido)propanamido)acrylate as white solid. LCMS (22010199D TFA LCMS-5 C₈) RT: 1.453 min; area % (215 nm): 94.9%; m z=475.2 [M+H]⁺. ¹H NMR (400 MHz, CDCl₃) δ 8.54 (s, 1H), 8.08 (s, 1H), 7.90-7.80 (m, 3H), 7.49-7.42 (m, 2H), 6.70 (s, 1H), 6.59 (s, 1H), 5.91 (d, J=1.4 Hz, 1H), 4.77 (p, J=7.1 Hz, 1H), 3.81 (s, 3H), 1.56 (d, J=7.0 Hz, 3H), 1.52 (s, 9H).

### Example 16: Compound 58

### Methyl (2-(2-(4-((tert-butoxy carbonyl)amino)phenyl)thiazole-4-carboxamido)acryloyl)glycinate

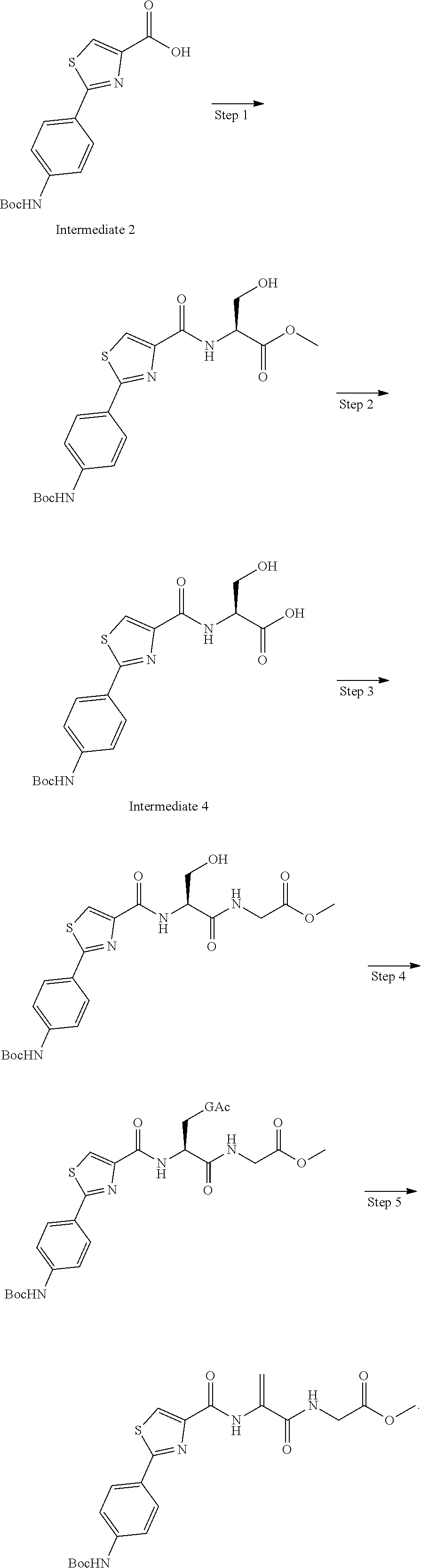

Step 1. Methyl (2-(4-((tert-butoxycarbonyl)amino)phenyl)thiazole-4-carbonyl)serinate was prepared following General experimental procedure 3.2-4 ((tert-butoxycarbonyl)amino)phenyl)thiazole-4-carboxylic acid (Intermediate 2) (1.0 g, 3.2 mmol) and L-Ser-OMe.HCl (0.59 g, 1.2 eq., 3.8 mmol) gave methyl (2-(4-((tert-butoxycarbonyl)amino)phenyl)thiazole-4-carbonyl)serinate (0.88 g, 2.1 mmol, 660%). LCMS (General 3 basic) RT: 1.54 min; area % (254 nm): 64%; m z=420.2 [M−H]⁺.

Step 2. (2-(4-((tert-butoxycarbonyl)amino)phenyl)thiazole-4-carbonyl)serine (Intermediate 4) was prepared following General experimental procedure 2. Methyl (2-(4-((tert-butoxycarbonyl)amino)phenyl)thiazole-4-carbonyl)serinate (0.88 g, 2.1 mmol) gave (2-(4-((tert-butoxycarbonyl)amino)phenyl)thiazole-4-carbonyl)serine (Intermediate 4) (0.84 mg, 2.1 mmol, 99%) as a white solid.

Step 3. Methyl (2-(4-((tert-butoxycarbonyl)amino)phenyl)thiazole-4-carbonyl)serylglycinate was prepared following General experimental procedure 3. (2-(4-((tert-butoxycarbonyl)amino)phenyl)thiazole-4-carbonyl)serine (Intermediate 4) (0.42 g, 1.0 mmol) and H-Gly-OMe.HCl (0.18 g, 1.4 mmol) gave methyl (2-(4-((tert-butoxycarbonyl)amino)phenyl)thiazole-4-carbonyl)serylglycinate (0.35 g, 0.72 mmol, 70%) as a yellow solid. ¹H NMR (400 MHz, MeOD) δ 8.16 (s, 1H), 7.98-7.88 (m, 2H), 7.59-7.51 (m, 2H), 4.71 (t, J=4.9 Hz, 1H), 4.04-3.96 (m, 3H), 3.92 (dd, J=11.3, 4.8 Hz, 1H), 3.73 (s, 3H), 1.54 (s, 9H).

Step 4. Methyl O-acetyl-N-(2-(4-((tert-butoxycarbonyl)amino)phenyl)thiazole-4-carbonyl)serylglycinate was prepared following General experimental procedure 5. Methyl (2-(4-((tert-butoxycarbonyl)amino)phenyl)thiazole-4-carbonyl)serylglycinate (0.13 mg, 0.27 mmol) and acetic anhydride (26 μL, leq., 0.27 mmol) gave methyl O-acetyl-N-(2-(4-((tert-butoxycarbonyl)amino)phenyl)thiazole-4-carbonyl)serylglycinate (153 mg, quant). LCMS (General 3 acidic) RT: 1.14 min; area % (254 nm): 92%; m z=521.5 [M+H]⁺.

Step 5. Methyl (2-(2-(4-((tert-butoxycarbonyl)amino)phenyl)thiazole-4-carboxamido)acryloyl) glycinate was prepared using the following procedure. To a solution of methyl (2-(2-(4-((tert-butoxycarbonyl)amino)phenyl)thiazole-4-carboxamido)acryloyl)glycinate (51 mg, 99 μmol) in CH₂Ch (1 mL) was added DBU (30 μL, 2 eq., 197 μmol) and the mixture was stirred at room temperature overnight. The reaction mixture was concentrated in vacuo and the crude material was purified by automated reverse phase FCC to provide methyl (2-(2-(4-((tert-butoxycarbonyl)amino)phenyl)thiazole-4-carboxamido)acryloyl)glycinate (12.5 mg, 27.1 μmol, 28%) as a white solid. LCMS (22010199D TFA LCMS-5 C8) RT: 1.403 min; area % (215 nm): 93.2%; m z=461.2 [M+H]⁺. ¹H NMR (400 MHz, CDCl₃) δ 9.98 (s, 1H), 8.06 (s, 1H), 7.94-7.82 (m, 2H), 7.48-7.39 (m, 2H), 6.73 (s, 1H), 6.71-6.66 (m, 2H), 5.43 (t, J=1.7 Hz, 1H), 4.18 (d, J=5.0 Hz, 2H), 3.79 (s, 3H), 1.52 (s, 9H).

### Example 17: Compound 59

### Methyl (2-(2-(4-((tert-butoxycarbonyl)amino)phenyl)thiazole-4-carboxamido)acryloyl)-L-alaninate

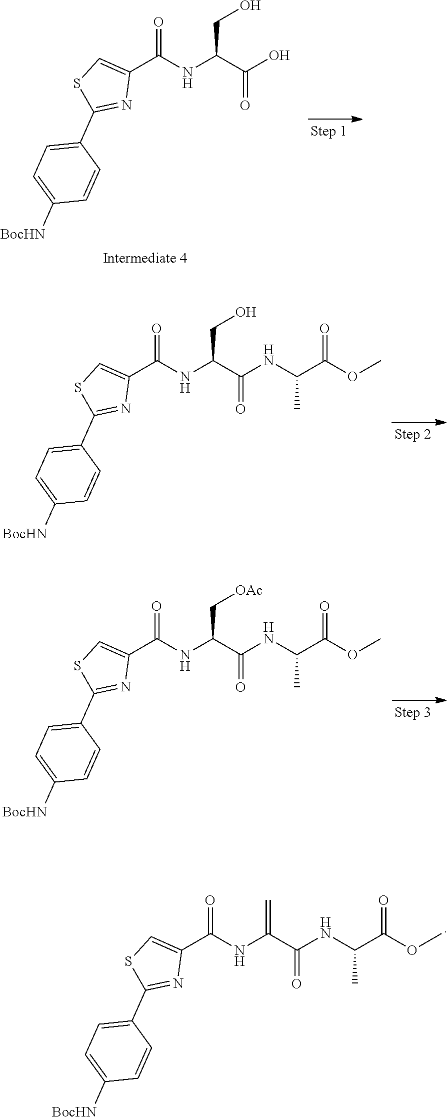 [SMILES: CC(C)(C)OC(=O)Nc1ccc(-c2nc(C(=O)N[C@@H](CO)C(=O)O)cs2)cc1 ; COC(=O)[C@H](C)NC(=O)[C@H](CO)NC(=O)c1csc(-c2ccc(NC(=O)OC(C)(C)C)cc2)n1 ; COC(=O)[C@H](C)NC(=O)[C@H](COC(C)=O)NC(=O)c1csc(-c2ccc(NC(=O)OC(C)(C)C)cc2)n1 ; C=C(NC(=O)c1csc(-c2ccc(NC(=O)OC(C)(C)C)cc2)n1)C(=O)N[C@@H](C)C(=O)OC]

Step 1. Methyl (2-(4-((tert-butoxycarbonyl)amino)phenyl)thiazole-4-carbonyl)seryl-L-alaninate was prepared following General experimental procedure 3. (2-(4-((tert-butoxycarbonyl)amino) phenyl)thiazole-4-carbonyl)serine (Intermediate 4) (0.42 g, 1.0 mmol) and H-Ala-OMe.HCl (0.19 g, 1.3 eq., 1.3 mmol) gave methyl (2-(4-((tert-butoxycarbonyl)amino)phenyl)thiazole-4-carbonyl)seryl-L-alaninate (0.24 g, 0.49 mmol, 48%) as a yellow foam. LCMS (General 3 basic) RT: 1.60 min; area % (254 nm): 81%; m z=493.2 [M+H]⁺.

Step 2. Methyl O-acetyl-N-(2-(4-((tert-butoxycarbonyl)amino)phenyl)thiazole-4-carbonyl)-L-seryl-L-alaninate was prepared following General experimental procedure 5. Methyl (2-(4-((tert-butoxycarbonyl)amino)phenyl)thiazole-4-carbonyl)-L-seryl-L-alaninate (0.20 g, 0.41 mmol) and acetic anhydride (42 μL, 0.44 mmol) gave methyl O-acetyl-N-(2-(4-((tert-butoxycarbonyl)amino)phenyl)thiazole-4-carbonyl)-L-seryl-L-alaninate (0.23 g, 0.44 mmol, quant.) as a light yellow foam. LCMS (General 3 basic) RT: 1.59 min; area % (254 nm): 86%; m z=535.2 [M+H]⁺.

Step 3. Methyl (2-(2-(4-((tert-butoxycarbonyl)amino)phenyl)thiazole-4-carboxamido)acryloyl)-L-alaninate was prepared using the following procedure. To a solution of methyl O-acetyl-N-(2-(4-((tert-butoxycarbonyl)amino)phenyl)thiazole-4-carbonyl)-L-seryl-L-alaninate (52 mg, 98 μmol) in CH₂Ch (1 mL) was added DBU (30 μL, 0.20 mmol) and the resulting mixture was stirred for 16 hours at room temperature. The reaction mixture was concentrated in vacuo and the crude material was purified by automated reverse phase FCC to provide methyl (2-(2-(4-((tert-butoxycarbonyl)amino)phenyl)thiazole-4-carboxamido)acryloyl)-L-alaninate (10.8 mg, 22.8 μmol, 23%). LCMS (22010199D TFA LCMS-5 C₈) RT: 1.472 min; area % (215 nm): 97.4%; m/z=475.2 [M+H]⁺. ¹H NMR (400 MHz, CDCl₃) δ 9.99 (s, 1H), 8.05 (s, 1H), 7.90 (d, J=8.4 Hz, 2H), 7.43 (d, J=8.3 Hz, 2H), 6.80 (d, J=7.2 Hz, 1H), 6.72 (s, 1H), 6.67 (d, J=1.8 Hz, 1H), 5.44-5.39 (m, 1H), 4.69 (p, J=7.2 Hz, 1H), 3.79 (s, 3H), 1.50 (m, 12H).

### Example 18: Compound 60

### N₃(CH₂CH₂O)₃CH₂C(O)-Cha-D-Arg-Cha-D-Arg-Cha-D-Arg-NH₂ HCl salt

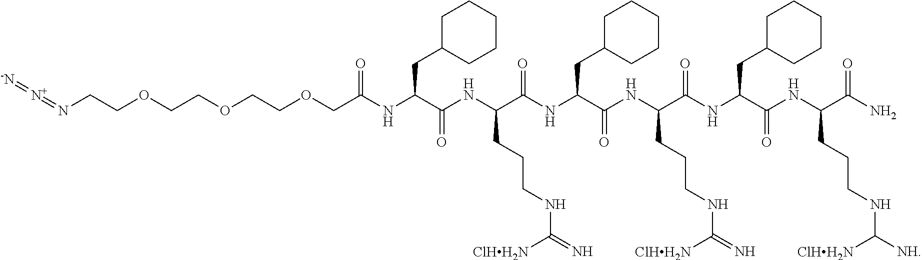

N₃(CH₂CH₂O)₃CH₂C(O)-Cha-D-Arg-Cha-D-Arg-Cha-D-Arg-NH₂ HCl salt was prepared following General procedure for solid phase peptide synthesis on 0.25 mmol scale. Purification using HCl buffers provided the desired HCl salt form.
N₃(CH₂CH₂O)₃CH₂C(O)-Cha-D-Arg-Cha-D-Arg-Cha-D-Arg-NH₂ HCl salt (53 mg, 42 μmol, 17%) was isolated as a white solid. LCMS (30833 LCMS-6): RT: 2.365 min; Area: 83.7% (215 nm), 99.8% (ELSD); m z=1160.8 [M+H]+

### Example 19: Compound 61

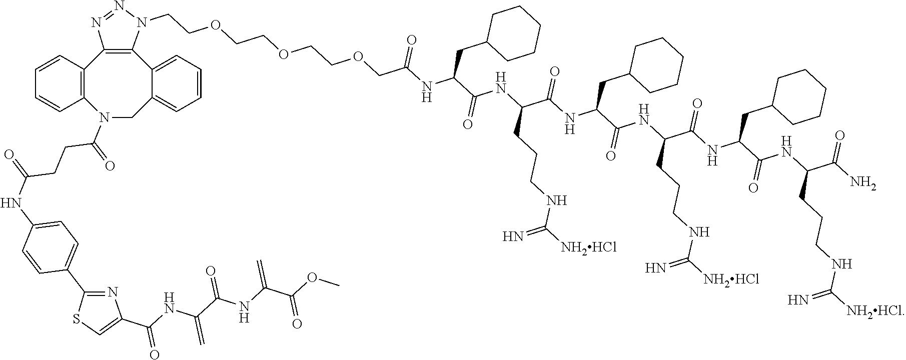

Compound 61 was prepared following General procedure for conjugation chemistry between azido-peptide and. Compound 49 (3.0 mg, 4.5 μmol) and Compound 60 (5.8 mg, 4.5 μmol) gave Compound 61 (4.5 mg, 2.3 μmol, 51%) as a white solid. LCMS (30833 LCMS-6): RT: 2.781 min; Area: 91.1% (215 nm), 99.4% (ELSD); m/z=1820.1 [M+H]⁺.

### Example 20: Compound 62

### Methyl (E)-2-(2-(2-(4-((tert-butoxycarbonyl)amino)phenyl)thiazole-4-carboxamido)acrylamido)but-2-enoate

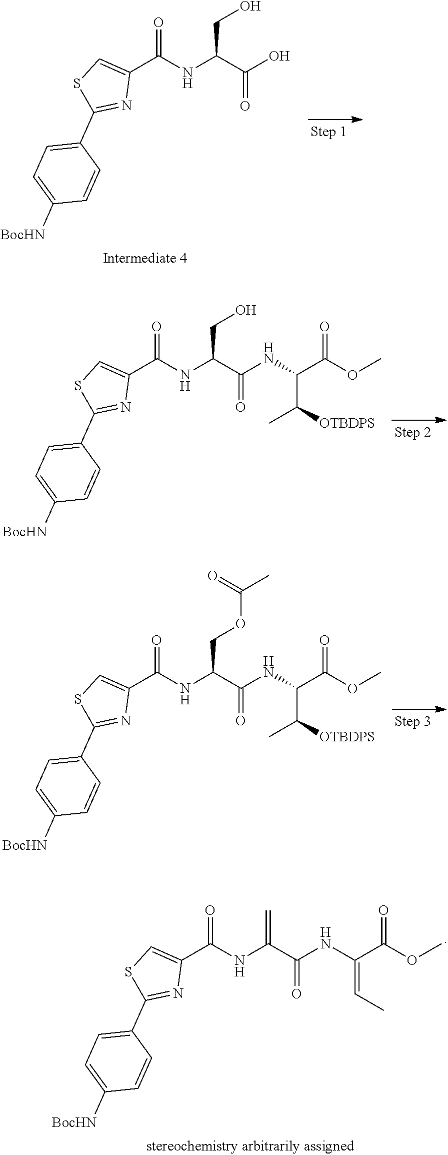

Step 1. Methyl N-((2-(4-((tert-butoxycarbonyl)amino)phenyl)thiazole-4-carbonyl)seryl)-O-(tert-butyldiphenylsilyl)-L-allothreoninate was prepared following General experimental procedure 4. (2-(4-((tert-butoxycarbonyl)amino)phenyl)thiazole-4-carbonyl)serine (Intermediate 4) (785 mg, 1.93 mmol) and methyl O-(tert-butyldiphenylsilyl)-L-allothreoninate (859 mg, 1.2 eq., 2.31 mmol) gave methyl N-((2-(4-((tert-butoxycarbonyl)amino)phenyl)thiazole-4-carbonyl)seryl)-O-(tert-butyldiphenylsilyl)-L-allothreoninate (1.54 g, 2.02 mmol, quant.) as a viscous oil. LCMS (General 3 acidic) RT: 1.81 min; area % (254 nm): 96%; m z=761.3 [M+H]⁺.

Step 2. Methyl N—(O-acetyl-N-(2-(4-((tert-butoxycarbonyl)amino)phenyl)thiazole-4-carbonyl)seryl)-O-(tert-butyldiphenylsilyl)-L-allothreoninate was prepared following General experimental procedure 5. Methyl N-((2-(4-((tert-butoxycarbonyl)amino)phenyl)thiazole-4-carbonyl)seryl)-O-(tert-butyldiphenylsilyl)-L-allothreoninate (1.6 g, 2.1 mmol) and acetic anhydride (0.22 mL, 1.1 eq., 2.3 mmol) gave methyl N—(O-acetyl-N-(2-(4-((tert-butoxycarbonyl)amino)phenyl)thiazole-4-carbonyl)seryl)-O-(tert-butyldiphenylsilyl)-L-allothreoninate (1.37 g, 1.71 mmol, 81%) as a yellow solid. LCMS (General 3 acidic) RT: 1.88 min; area % (254 nm): 75%; m z=803.5 [M+H]⁺.

Step 3. Methyl (E)-2-(2-(2-(4-((tert-butoxycarbonyl)amino)phenyl)thiazole-4-carboxamido) acrylamido)but-2-enoate was prepared following General experimental procedure 7. Methyl N—(O-acetyl-N-(2-(4-((tert-butoxycarbonyl)amino)phenyl)thiazole-4-carbonyl)seryl)-O-(tert-butyldiphenylsilyl)-L-allothreoninate (306 mg, 0.38 mmol) gave methyl methyl (E)-2-(2-(2-(4-((tert-butoxycarbonyl)amino)phenyl)thiazole-4-carboxamido)acrylamido)but-2-enoate (4.6 mg, 9.5 μmol, 2.5%) %). LCMS (22010199C TFA LCMS-5 C₃) RT: 1.470 min; area: 93.8% (215 nm); m/z 487.2 [M+H]⁺¹H NMR (400 MHz, CDCl₃) δ 9.98 (s, 1H), 8.17 (s, 1H), 8.06 (s, 1H), 7.95-7.88 (m, 2H), 7.44 (d, J=8.6 Hz, 2H), 7.33 (q, J=7.7 Hz, 2H), 6.70 (d, J=2.0 Hz, 1H), 6.59 (s, 1H), 5.42 (s, 1H), 3.88 (s, 3H), 2.15 (d, J=7.7 Hz, 3H), 1.52 (s, 9H).

### Example 21: Compound 63

### methyl (E)-2-(2-(2-(4-((tert-butoxycarbonyl)amino)phenyl)thiazole-4-carboxamido)acrylamido)but-2-enoate

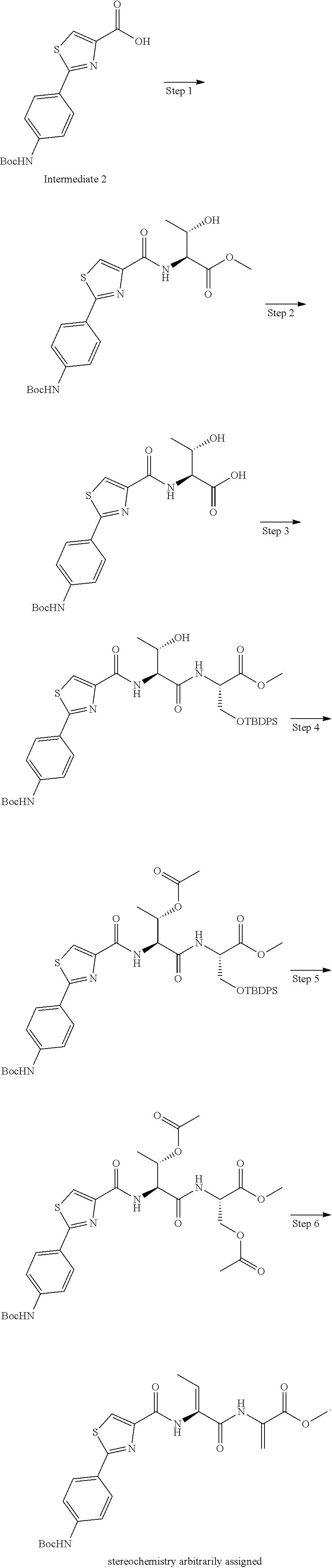

Step 1. Methyl (2-(4-((tert-butoxycarbonyl)amino)phenyl)thiazole-4-carbonyl)-L-allothreoninate was prepared following General experimental procedure 4. 2-(4-((tert-butoxycarbonyl)amino)phenyl)thiazole-4-carboxylic acid (Intermediate 2) (0.98 g, 3.1 mmol) and methyl L-allothreoninate hydrochloride (0.57 g, 1.1 eq., 3.4 mmol) gave methyl (2-(4-((tert-butoxycarbonyl)amino)phenyl)thiazole-4-carbonyl)-L-allothreoninate (1.6 g, 3.2 mmol, quant.) as a white solid.

Step 2. (2-(4-((tert-butoxycarbonyl)amino)phenyl)thiazole-4-carbonyl)-L-allothreonine was prepared following General experimental procedure 2. Methyl (2-(4-((tert-butoxycarbonyl)amino)phenyl)thiazole-4-carbonyl)-L-allothreoninate (1.6 g, 3.2 mmol) gave (2-(4-((tert-butoxycarbonyl)amino)phenyl)thiazole-4-carbonyl)-L-allothreonine (1.4 g, 3.2 mmol, quant.) as a white solid.

Step 3. Methyl N-((2-(4-((tert-butoxycarbonyl)amino)phenyl)thiazole-4-carbonyl)-L-all othreonyl)-O-(tert-butyl diphenyl silyl)-L-serinate was prepared following General experimental procedure 4. (2-(4-((tert-butoxycarbonyl)amino)phenyl)thiazole-4-carbonyl)-L-allothreonine (1.4 g, 3.2 mmol) and methyl O-(tert-butyldiphenylsilyl)-L-serinate hydrochloride (1.4 g, 1.1 eq., 3.5 mmol) gave methyl N-((2-(4-((tert-butoxycarbonyl)amino)phenyl)thiazole-4-carbonyl)-L-allothreonyl)-O-(tert-butyldiphenylsilyl)-L-serinate (2.3 g, 3.0 mmol, 93%)

Step 4. Methyl N—(O-acetyl-N-(2-(4-((tert-butoxycarbonyl)amino)phenyl)thiazole-4-carbonyl)-L-allothreonyl)-O-(tert-butyldiphenylsilyl)-L-serinate was prepared following General experimental procedure 5. Methyl N-((2-(4-((tert-butoxycarbonyl)amino)phenyl) thiazole-4-carbonyl)-L-allothreonyl)-O-(tert-butyldiphenylsilyl)-L-serinate (2.3 g, 3.0 mmol) gave methyl N—(O-acetyl-N-(2-(4-((tert-butoxycarbonyl)amino)phenyl)thiazole-4-carbonyl)-L-allothreonyl)-O-(tert-butyldiphenylsilyl)-L-serinate (2.2 g, 2.7 mmol, 90%).

Step 5. Methyl O-acetyl-N—(O-acetyl-N-(2-(4-((tert-butoxycarbonyl)amino)phenyl)thiazole-4-carbonyl)-L-allothreonyl)-L-serinate was prepared using the following procedure. To a solution of methyl N—(O-acetyl-N-(2-(4-((tert-butoxycarbonyl)amino)phenyl)thiazole-4-carbonyl)-L-all othreonyl)-O-(tert-butyldiphenylsilyl)-L-serinate (0.30 g, 0.37 mmol) in THE (6 mL) was added a solution of TBAF in THE (0.56 mL, 1M, 1.5 eq., 0.56 mmol) and the resulting mixture was stirred for 1 hour. The reaction mixture was concentrated in vacuo and the residue was dissolved in MeCN (6 mL). Triethylamine (0.13 mL, 2.5 eq., 0.93 mmol) and acetic anhydride (48 μL, 1.35 eq., 0.50 mmol) were added and the resulting mixture was stirred for 45 minutes at room temperature. Water was added and the mixture was concentrated in vacuo to remove MeCN. Then, the mixture was extracted with EtOAc. The organic layer was washed with brine, dried over Na₂SO₄, filtered and concentrated. The crude material was purified by automated FCC to provide a methyl O-acetyl-N—(O-acetyl-N-(2-(4-((tert-butoxycarbonyl)amino)phenyl)thiazole-4-carbonyl)-L-allothreonyl)-L-serinate (0.16 g, 0.27 mmol, 72%) as a white solid.

Step 6. Methyl (Z)-2-(2-(2-(4-((tert-butoxycarbonyl)amino)phenyl)thiazole-4-carboxamido)but-2-enamido)acrylate (E/Z stereochemistry arbitrarily assigned) was prepared following General experimental procedure 8. Methyl O-acetyl-N—(O-acetyl-N-(2-(4-((tert-butoxycarbonyl)amino)phenyl)thiazole-4-carbonyl)-L-allothreonyl)-L-serinate (0.15 g, 0.25 mmol) gave methyl (Z)-2-(2-(2-(4-((tert-butoxycarbonyl)amino)phenyl)thiazole-4-carboxamido)but-2-enamido)acrylate (30 mg, 62 μmol, 25%) as a white solid. %). LCMS (22010199C TFA LCMS-5 C₃) RT: 1.387 min; Area: 99.6% (215 nm), 99.3% (306 nm); m/z 487.2 [M+H]⁺. ¹H NMR (400 MHz, CDCl₃) δ 8.73 (s, 1H), 8.48 (s, 1H), 8.13 (s, 1H), 7.95-7.88 (m, 2H), 7.48 (d, J=8.5 Hz, 2H), 6.73 (q, J=7.1 Hz, 1H), 6.69 (s, 1H), 6.63 (s, 1H), 5.94 (d, J=1.4 Hz, 1H), 3.81 (s, 3H), 1.89 (d, J=7.1 Hz, 3H), 1.54 (s, 9H).

### Example 22: Compound 67

### Methyl 2-(2-(2-(4-((tert-butoxycarbonyl)amino)phenyl)thiazole-5-carboxamido)acrylamido)acrylate

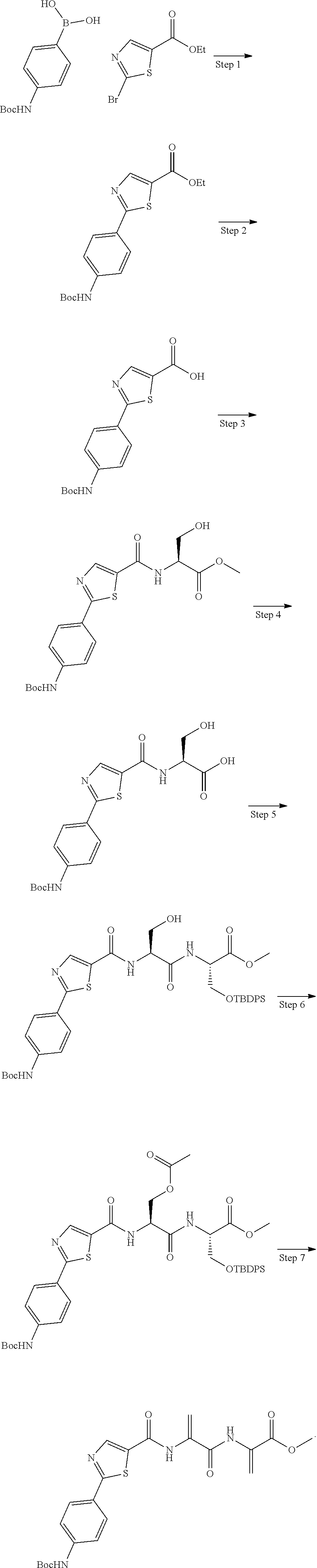

Step 1. ethyl 2-(4-((tert-butoxycarbonyl)amino)phenyl)thiazole-5-carboxylate was prepared following General experimental procedure 1. (4-((tert-butoxycarbonyl)amino)phenyl)boronic acid (2.0 g, 8.4 mmol) and ethyl 2-bromothiazole-5-carboxylate (2.0 g, 8.4 mmol) gave ethyl 2-(4-((tert-butoxycarbonyl)amino)phenyl)thiazole-5-carboxylate (1.26 g, 3.62 mmol, 43%) as a yellow solid. ¹H NMR (299 MHz, CDCl₃) δ 8.39 (s, 1H), 7.94 (d, J=8.8 Hz, 2H), 7.49 (d, J=8.6 Hz, 2H), 6.66 (s, 1H), 4.40 (q, J=7.1 Hz, 2H), 1.55 (d, J=1.0 Hz, 10H), 1.42 (t, J=7.1 Hz, 3H).

Step 2. 2-(4-((tert-butoxycarbonyl)amino)phenyl)thiazole-5-carboxylic acid was prepared following General experimental procedure 2. Ethyl 2-(4-((tert-butoxycarbonyl)amino)phenyl)thiazol e-5-carboxylmate (1.2 g, 3.4 mmol) gave 2-(4-((tert-butoxycarbonyl)amino)phenyl)thiazole-5-carboxylic acid (1.0 g, 3.1 mmol, 91%) as a white solid.

Step 3. methyl (2-(4-((tert-butoxycarbonyl)amino)phenyl)thiazole-5-carbonyl)-L-serinate was prepared following General experimental procedure 4. 2-(4-((tert-butoxycarbonyl)amino)phenyl)thiazole-5-carboxylic acid (1.6 g, 5.0 mmol) and H-Ser-OMe.HCl (0.93 g, 1.2 eq., 6.0 mmol) gave methyl (2-(4-((tert-butoxycarbonyl)amino)phenyl)thiazole-5-carbonyl)-L-serinate (1.4 g, 3.3 mmol, 67%) as an orange oil. LCMS (General 3 acidic) RT: 0.99 min; area % (254 nm): 88%; m z=422.3 [M+H]⁺.

Step 4. (2-(4-((tert-butoxycarbonyl)amino)phenyl)thiazole-5-carbonyl)-L-serine was prepared following General experimental procedure 2. Methyl (2-(4-((tert-butoxycarbonyl)amino)phenyl)thiazole-5-carbonyl)-L-serinate (1.4 g, 3.3 mmol) gave (2-(4-((tert-butoxycarbonyl)amino)phenyl)thiazole-5-carbonyl)-L-serine (0.98 g, 2.4 mmol, 72%).

Step 5. Methyl N-((2-(4-((tert-butoxycarbonyl)amino)phenyl)thiazole-5-carbonyl)-L-seryl)-O-(tert-butyldiphenylsilyl)-L-serinate was prepared following General experimental procedure 4. (2-(4-((tert-butoxycarbonyl)amino)phenyl)thiazole-5-carbonyl)-L-serine (0.98 g, 2.4 mmol) and methyl O-(tert-butyldiphenylsilyl)-L-serinate hydrochloride (1.1 g, 1.2 eq. 2.9 mmol) gave crude methyl N-((2-(4-((tert-butoxycarbonyl)amino)phenyl)thiazole-5-carbonyl)-L-seryl)-O-(tert-butyldiphenylsilyl)-L-serinate (2.25 g) which was used as such in the next step

Step 6. Methyl N—(O-acetyl-N-(2-(4-((tert-butoxycarbonyl)amino)phenyl)thiazole-5-carbonyl)-L-seryl)-O-(tert-butyldiphenylsilyl)-L-serinate was prepared following General experimental procedure 5. Crude methyl N-((2-(4-((tert-butoxycarbonyl)amino)phenyl)thiazole-5-carbonyl)-L-seryl)-O-(tert-butyldiphenylsilyl)-L-serinate (2.25 g) and acetic anhydride (0.34 mL, 3.6 mmol) gave methyl N—(O-acetyl-N-(2-(4-((tert-butoxycarbonyl)amino)phenyl)thiazole-5-carbonyl)-L-seryl)-O-(tert-butyldiphenylsilyl)-L-serinate (0.40 g, 0.51 mmol, 21% ver 2 steps). LCMS (General 3 acidic) RT: 1.78 min; area % (214 nm): 100%; m z=789.6 [M+H]⁺.

Step 7. Methyl 2-(2-(2-(4-((tert-butoxycarbonyl)amino)phenyl)thiazole-5-carboxamido) acrylamido)acrylate was prepared following General experimental procedure 7. Methyl N—(O-acetyl-N-(2-(4-((tert-butoxycarbonyl)amino)phenyl)thiazole-5-carbonyl)-seryl)-O-(tert-butyldiphenylsilyl)-L-serinate (0.19 g, 0.24 mmol) gave methyl 2-(2-(2-(4-((tert-butoxycarbonyl)amino)phenyl)thiazole-5-carboxamido)acrylamido)acrylate (7 0.8 mg, 16 μmol, 27%) as a white solid LC-MS (22010199A TFA LCMS-5 Cl): RT: 1.353 min; Area 97.7% (215 nm), 96.7% (304 nm); m/z=473.2 [M+H]⁺. ¹H NMR (400 MHz, CDCl₃) δ (ppm) 8.66 (s, 1H), 8.57 (s, 1H), 8.24 (s, 1H), 7.92 (d, J=8.8 Hz, 2H), 7.48 (d, J=8.8 Hz, 2H), 6.72 (d, J=2.5 Hz, 1H), 6.67 (s, 1H), 6.63 (s, 1H), 6.04 (d, J=1.0 Hz, 1H), 5.49 (dd, J=2.3 Hz, J=1.3 Hz, 1H), 3.91 (s, 1H), 1.54 (s, 9H).

### Example 23: Compound 68

### Methyl 2-(2-(5-(4-((tert-butoxycarbonyl)amino)phenyl)thiophene-3-carboxamido)acrylamido)acrylate

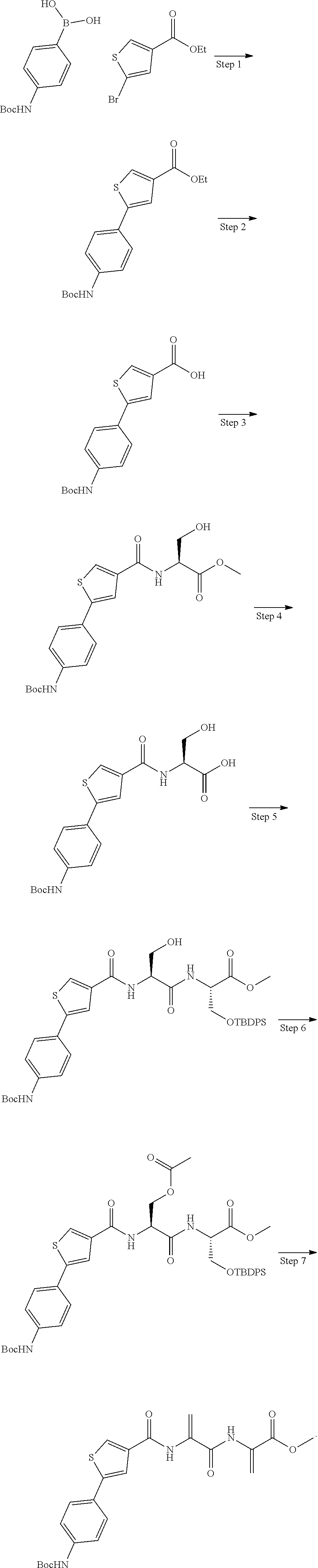

Step 1. Ethyl 5-(4-((tert-butoxycarbonyl)amino)phenyl)thiophene-3-carboxylate was prepared following General experimental procedure 1. Ethyl 5-bromothiophene-3-carboxylate (1.0 g, 4.2 mmol) and (4-((tert-butoxycarbonyl)amino)phenyl)boronic acid (1.0 g, 4.2 mmol) gave ethyl 5-(4-((tert-butoxycarbonyl)amino)phenyl)thiophene-3-carboxylate (1.1 g, 3.2 mmol, 76%) as white solid. ¹H NMR (299 MHz, CDCl₃) δ 8.00 (m, 1H), 7.65 (m, 1H), 7.55 (d, J=8.7 Hz, 2H), 7.41 (d, J=8.5 Hz, 2H), 7.28 (s, 4H), 6.54 (s, 1H), 4.37 (q, J=7.1 Hz, 2H), 1.55 (s, 9H), 1.40 (t, J=7.1 Hz, 3H).

Step 2. 5-(4-((tert-butoxycarbonyl)amino)phenyl)thiophene-3-carboxylic acid was prepared following General experimental procedure 2. Ethyl 5-(4-((tert-butoxycarbonyl)amino)phenyl)thiophene-3-carboxylate (1.1 g, 3.2 mmol) and 5-(4-((tert-butoxycarbonyl)amino)phenyl)thiophene-3-carboxylic acid (0.90 g, 2.8 mmol, 89%) as a white solid ¹H NMR (299 MHz, CD30D) 6 8.08 (m, 1H), 7.65 (m, 1H), 7.62-7.52 (m, 2H), 7.48 (d, J=8.7 Hz, 2H), 1.54 (s, 8H).

Step 3. Methyl (5-(4-((tert-butoxycarbonyl)amino)phenyl)thiophene-3-carbonyl)-L-serinate was prepared following General experimental procedure 3. 5-(4-((tert-butoxycarbonyl)amino)phenyl)thiophene-3-carboxylic acid (0.90 g, 2.8 mmol) and methyl L-serinate hydrochloride (0.44 g, 2.8 mmol) gave methyl (5-(4-((tert-butoxycarbonyl)amino)phenyl)thiophene-3-carbonyl)-L-serinate (1.0 g, 2.4 mmol, 84%) as a yellow oil. ¹H NMR (299 MHz, CDCl₃) δ 7.84 (m, 1H), 7.56-7.46 (m, 3H), 7.40 (d, J=8.5 Hz, 2H), 7.03 (d, J=7.2 Hz, 1H), 6.64 (s, 1H), 4.87 (dt, J=7.2, 3.6 Hz, 1H), 4.39 (q, J=7.1 Hz, 1H), 4.09 (t, J=4.0 Hz, 2H), 3.85 (s, 3H), 1.55 (s, 9H).

Step 4. (5-(4-((tert-butoxycarbonyl)amino)phenyl)thiophene-3-carbonyl)-L-serine was prepared following General experimental procedure 2. Methyl (5-(4-((tert-butoxycarbonyl)amino)phenyl)thiophene-3-carbonyl)-L-serinate (1.0 g, 0.24 mmol) gave (5-(4-((tert-butoxycarbonyl)amino)phenyl)thiophene-3-carbonyl)-L-serine (0.90 g, 2.2 mmol, 93%) as a yellow solid. ¹H NMR (299 MHz, CD30D) 6 9.05 (s, 1H), 8.25 (d, J=8.0 Hz, OH), 8.04 (d, J=1.4 Hz, 1H), 7.74 (d, J=1.4 Hz, 1H), 7.63-7.52 (m, 2H), 7.48 (d, J=8.6 Hz, 2H), 4.76-4.67 (m, 1H), 4.10-3.92 (m, 2H), 1.55 (s, 9H).

Step 5. Methyl N-((5-(4-((tert-butoxycarbonyl)amino)phenyl)thiophene-3-carbonyl)-L-seryl)-O-(tert-butyldiphenylsilyl)-L-serinate was prepared following General experimental procedure 4. (5-(4-((tert-butoxycarbonyl)amino)phenyl)thiophene-3-carbonyl)-L-serine (0.20 g, 0.49 mmol) and methyl O-(tert-butyldiphenylsilyl)-L-serinate hydrochloride (0.23 g, 1.2 eq., 0.59 mmol) gave crude methyl N-((5-(4-((tert-butoxycarbonyl)amino)phenyl)thiophene-3-carbonyl)-L-seryl)-O-(tert-butyldiphenylsilyl)-L-serinate (233 mg) which was used without purification in the next step.

Step 6. Methyl N—(O-acetyl-N-(5-(4-((tert-butoxycarbonyl)amino)phenyl)thiophene-3-carbonyl)-L-seryl)-O-(tert-butyldiphenylsilyl)-L-serinate was prepared following General experimental procedure 5. Crude methyl N-((5-(4-((tert-butoxycarbonyl)amino)phenyl)thiophene-3-carbonyl)-L-seryl)-O-(tert-butyldiphenylsilyl)-L-serinate (233 mg) gave methyl N—(O-acetyl-N-(5-(4-((tert-butoxycarbonyl)amino)phenyl)thiophene-3-carbonyl)-L-seryl)-O-(tert-butyldiphenylsilyl)-L-serinate (0.10 g, 0.13 mmol, 26% ver 2 steps)

Step 7. Methyl 2-(2-(5-(4-((tert-butoxycarbonyl)amino)phenyl)thiophene-3-carboxamido)acrylamido)acrylate was prepared following General experimental procedure 7. Methyl N—(O-acetyl-N-(5-(4-((tert-butoxycarbonyl)amino)phenyl)thiophene-3-carbonyl)-L-seryl)-O-(tert-butyldiphenylsilyl)-L-serinate (50 mg, 63 μmol) gave methyl 2-(2-(5-(4-((tert-butoxycarbonyl)amino)phenyl)thiophene-3-carboxamido)acrylamido)acrylate (3.9 mg, 8.3 μmol, 31%) as a white solid. LC-MS (22010199A TFA LCMS-5 Cl): RT 1.483 min; Area 84.0% (215 nm), 88.7% (304 nm); m z=472.0 [M+H]+

### Example 24: Compound 69

### Methyl 2-(2-(4′-((tert-butoxycarbonyl)amino)-[1,1′-biphenyl]-4-carboxamido)acrylamido)acrylate

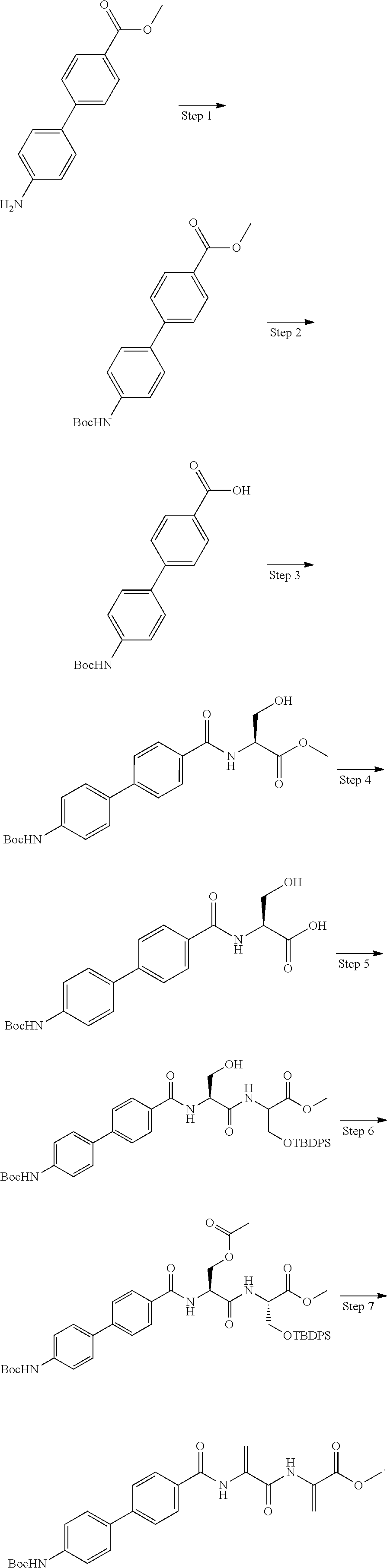

Step 1. Methyl 4′-((tert-butoxycarbonyl)amino)-[1,1′-biphenyl]-4-carboxylate was prepared using the following procedure. To a solution of methyl 4′-amino-[1,1′-biphenyl]-4-carboxylate (0.50 g, 2.2 mmol) in CH₂Cl₂ (20 mL) were added DMAP (0.54 g, 2 eq., 4.4 mmol) and Boc20 (0.48 g, 1 eq., 2.2 mmol) and the resulting mixture was stirred for 16 hours at room temperature. The mixture was filtered and HCl (1M, aq. 50 mL) was added. The mixture was extracted with EtOAc and the organic layer was dried over Na₂SO₄, filtered and concentrated. The crude material was purified by FCC to provide methyl 4′-((tert-butoxycarbonyl)amino)-[1, 1′-biphenyl]-4-carboxylate (0.30 g, 0.92 mmol, 42%). ¹H NMR (299 MHz, CDCl₃) δ 8.10 (m, 2H), 7.62 (m, 5H), 7.53-7.44 (m, 2H), 6.70 (s, 1H), 3.95 (s, 3H), 1.55 (s, 9H).

Step 2. 4′-((tert-butoxycarbonyl)amino)-[1, 1′-biphenyl]-4-carboxylic acid was prepared following General experimental procedure 2. Methyl 4′-((tert-butoxycarbonyl)amino)-[1,1′-biphenyl]-4-carboxylate (0.52 g, 1.6 mmol) gave 4′-((tert-butoxycarbonyl)amino)-[1,1′-biphenyl]-4-carboxylic acid (0.34 g, 1.1 mmol, 66%)¹H NMR (299 MHz, CD30D) 6 8.13-8.00 (m, 2H), 7.77-7.58 (m, 4H), 7.53 (m, 2H), 1.55 (s, 9H).

Step 3. Methyl (4′-((tert-butoxycarbonyl)amino)-[1,1′-biphenyl]-4-carbonyl)-L-serinate was prepared following General experimental procedure 4. 4′-((tert-butoxycarbonyl)amino)-[1,1′-biphenyl]-4-carboxylic acid (90 mg, 0.29 mmol) and methyl L-serinate hydrochloride (90 mg, 2 eq., 0.57 mmol) gave methyl (4′-((tert-butoxycarbonyl)amino)-[1,1′-biphenyl]-4-carbonyl)-L-serinate (34 mg, 82 μmol, 29%). LCMS (General 3 acidic) RT: 1.07 min; area % (254 nm): 100%; m/z=415.1 [M+H]⁺.

Step 4. (4′-((tert-butoxycarbonyl)amino)-[1, 1′-biphenyl]-4-carbonyl)-L-serine was prepared following General experimental procedure 2. Methyl (4′-((tert-butoxycarbonyl)amino)-[1,1′-biphenyl]-4-carbonyl)-L-serinate (34 mg, 82 μmol) gave crude (4′-((tert-butoxycarbonyl)amino)-[1,1′-biphenyl]-4-carbonyl)-L-serine (77 mg) which was used as such in the next step. LCMS (General 3 acidic) RT: 0.95 min; area % (254 nm): 82%; m z=401.3 [M+H]⁺.

Step 5. Methyl N-((4′-((tert-butoxycarbonyl)amino)-[1, 1′-biphenyl]-4-carbonyl)-L-seryl)-O-(tert-butyldiphenylsilyl)-L-serinate was prepared following General experimental procedure 4. (4′-((tert-butoxycarbonyl)amino)-[1, 1′-biphenyl]-4-carbonyl)-L-serine (100 mg, 0.25 mmol) and methyl O-(tert-butyldiphenylsilyl)-L-serinate hydrochloride (118 mg, 0.30 mmol) gave methyl N-((4′-((tert-butoxycarbonyl)amino)-[1, 1′-biphenyl]-4-carbonyl)-L-seryl)-O-(tert-butyldiphenylsilyl)-L-serinate (66 mg, 89 μmol, 36%). LCMS (General 3 acidic) RT: 1.72 min; area % (254 nm): 77%; m z=740.6 [M+H]⁺..

Step 6. Methyl N—(O-acetyl-N-(4′-((tert-butoxycarbonyl)amino)-[1, 1′-biphenyl]-4-carbonyl)-L-seryl)-O-(tert-butyldiphenylsilyl)-L-serinate was prepared following General experimental procedure 5. Methyl N-((4′-((tert-butoxycarbonyl)amino)-[1,1′-biphenyl]-4-carbonyl)-L-seryl)-O-(tert-butyldiphenylsilyl)-L-serinate (66 mg, 89 μmol) gave methyl N—(O-acetyl-N-(4′-((tert-butoxycarbonyl)amino)-[1,1′-biphenyl]-4-carbonyl)-L-seryl)-O-(tert-butyldiphenylsilyl)-L-serinate (44 mg, 56 μmol, 63%). LCMS (General 3 acidic) RT: 1.81 min; area % (254 nm): 100%; m/z=740.6 [M+H]⁺.

Step 7. Methyl 2-(2-(4′-((tert-butoxycarbonyl)amino)-[1,1′-biphenyl]-4-carboxamido)acrylamido)acrylate was prepared following General experimental procedure 7. Methyl N—(O-acetyl-N-(4′-((tert-butoxycarbonyl)amino)-[1, 1′-biphenyl]-4-carbonyl)-L-seryl)-O-(tert-butyldiphenylsilyl)-L-serinate (42 mg, 54 μmol) gave methyl 2-(2-(4′-((tert-butoxycarbonyl)amino)-[1, 1′-biphenyl]-4-carboxamido)acrylamido)acrylate (5.0 mg, 11 μmol, 20%). LC-MS (22010199A TFA LCMS-5 Cl): rt 1.483 min; Area 78.4% (215 nm), 83.7% (304 nm); m z=[M+H]⁺=466.2. ¹H NMR (CDCl₃) 6 (ppm) 8.92 (s, 1H), 8.60 (s, 1H), 7.92 (d, J=8.3 Hz, 2H), 7.67 (d, J=8.1 Hz, 2H), 7.57 (d, J=8.4 Hz, 2H), 7.47 (d, J=8.4 Hz, 2H), 6.82 (m, 1H), 6.67 (s, 1H), 6.56 (s, 1H), 6.03 (s, 1H), 5.49 (s, 1H), 3.91 (s, 3H), 1.54 (s, 9H).

### Example 25: Compound 70

### Methyl 2-(2-(2-(4-((tert-butoxycarbonyl)amino)-3-fluorophenyl)thiazole-4-carboxamido)acrylamido)acrylate

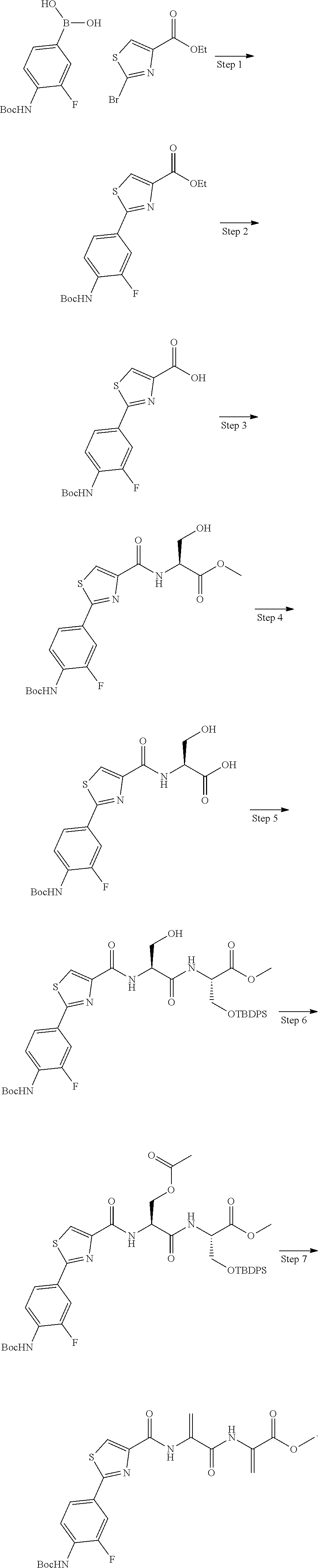

Step 1. Ethy 2-4-((tert-butoxycarboyl)amino)-3-fluorophenyl)thiazole-4-carboxylate was prepared following General experimental procedure 1. Ethyl 2-bromothiazole-4-carboxylate (0.20 g, 0.85 mmol) and (4-((tert-butoxycarbonyl)amino)-3-fluorophenyl)boronic acid (0.20 g, 0.78 mmol) gave ethyl 2-(4-((tert-butoxycarbonyl)amino)-3-fluorophenyl)thiazole-4-carboxylate (0.15 g, 0.42 mmol, 53%) ¹H NMR (400 MHz, CDCl₃) δ 8.20 (t, J=8.2 Hz, 1H), 8.10 (s, 1H), 7.79 (dd, J=11.8, 2.0 Hz, 1H), 7.68 (d, J=8.6 Hz, 1H), 6.82 (s, 1H), 4.43 (q, J=7.1 Hz, 2H), 1.52 (s, 9H), 1.41 (t, J=7.1 Hz, 3H).

Step 2. 2-(4-((tert-butoxycarbonyl)amino)-3-fluorophenyl)thiazole-4-carboxylic acid was prepared following General experimental procedure 2. Ethyl 2-(4-((tert-butoxycarbonyl)amino)-3-fluorophenyl)thiazole-4-carboxylate (0.71 g, 1.9 mmol) gave 2-(4-((tert-butoxycarbonyl)amino)-3-fluorophenyl)thiazole-4-carboxylic acid (0.63 g, 1.9 mmol, 97%). ¹H NMR (400 MHz, CDCl₃) δ 8.25 (d, J=8.3 Hz, 1H), 8.21 (s, 1H), 7.74 (dd, J=11.7, 2.0 Hz, 1H), 7.70-7.63 (m, 1H), 6.86 (d, J=3.6 Hz, 1H), 1.53 (s, 9H).

Step 3. Methyl (2-(4-((tert-butoxycarbonyl)amino)-3-fluorophenyl)thiazole-4-carbonyl)serinate was prepared following General experimental procedure 4. 2-(4-((tert-butoxycarbonyl)amino)-3-fluorophenyl)thiazole-4-carboxylic acid (0.63 g, 1.9 mmol) and methyl L-serinate hydrochloride (0.32 g, 1.1 eq., 2.1 mmol) gave methyl (2-(4-((tert-butoxycarbonyl)amino)-3-fluorophenyl)thiazole-4-carbonyl)serinate (0.87 g, purity 90%, 1.8 mmol, 96%)

Step 4. (2-(4-((tert-butoxycarbonyl)amino)-3-fluorophenyl)thiazole-4-carbonyl)serine was prepared following General experimental procedure 2. Methyl (2-(4-((tert-butoxycarbonyl)amino)-3-fluorophenyl)thiazole-4-carbonyl)serinate (0.87 g, purity 90%, 1.8 mmol) gave (2-(4-((tert-butoxycarbonyl)amino)-3-fluorophenyl)thiazole-4-carbonyl)serine (0.85 g, purity:90%, 1.8 mmol, quant.) as an off-white solid.

Step 5. Methyl N-((2-(4-((tert-butoxycarbonyl)amino)-3-fluorophenyl)thiazole-4-carbonyl)-L-seryl)-O-(tert-butyldiphenylsilyl)-L-serinaten was prepared following General experimental procedure 4. (2-(4-((tert-butoxycarbonyl)amino)-3-fluorophenyl)thiazole-4-carbonyl)serine (0.76 g, 1.8 mmol) and methyl O-(tert-butyldiphenylsilyl)-L-serinate hydrochloride (0.78 g, 1.1 eq., 2.0 mmol) gave methyl N-((2-(4-((tert-butoxycarbonyl)amino)-3-fluorophenyl)thiazole-4-carbonyl)-L-seryl)-O-(tert-butyldiphenylsilyl)-L-serinate (1.2 g, 1.5 mmol, 84%)

Step 6. Methyl N—(O-acetyl-N-(2-(4-((tert-butoxycarbonyl)amino)-3-fluorophenyl)thiazole-4-carbonyl)-L-seryl)-O-(tert-butyldiphenylsilyl)-L-serinate was prepared following General experimental procedure 5. Methyl N-((2-(4-((tert-butoxycarbonyl)amino)-3-fluorophenyl) thiazole-4-carbonyl)-L-seryl)-O-(tert-butyldiphenylsilyl)-L-serinate (1.2 g, 1.5 mmol) gave methyl N—(O-acetyl-N-(2-(4-((tert-butoxycarbonyl)amino)-3-fluorophenyl)thiazole-4-carbonyl)-L-seryl)-O-(tert-butyldiphenylsilyl)-L-serinate (1.1 g, 1.4 mmol, 93%). ¹H NMR (400 MHz, CDCl₃) δ 8.21 (t, J=8.2 Hz, 1H), 8.09 (d, J=7.7 Hz, 1H), 8.04 (s, 1H), 7.72 (dd, J=11.7, 2.0 Hz, 1H), 7.63 (d, J=8.4 Hz, 1H), 7.61-7.50 (m, 5H), 7.44-7.26 (m, 7H), 7.12 (d, J=8.1 Hz, 1H), 6.83 (d, J=3.5 Hz, 1H), 4.95-4.85 (m, 1H), 4.67 (dt, J=8.2, 2.9 Hz, 1H), 4.52 (dd, J=11.3, 5.6 Hz, 1H), 4.39 (dd, J=11.3, 6.2 Hz, 1H), 4.11 (dd, J=10.3, 2.7 Hz, 2H), 3.88 (dd, J=10.3, 3.0 Hz, 1H), 3.73 (s, 3H), 2.08 (s, 3H), 1.53 (s, 9H), 0.96 (s, 9H).

Step 7. Methyl 2-(2-(2-(4-((tert-butoxycarbonyl)amino)-3-fluorophenyl)thiazole-4-carboxamido) acrylamido)acrylate was prepared following General experimental procedure 7. Methyl N—(O-acetyl-N-(2-(4-((tert-butoxycarbonyl)amino)-3-fluorophenyl)thiazole-4-carbonyl)-L-seryl)-O-(tert-butyldiphenylsilyl)-L-serinate (0.30 g, 0.37 mmol) gave methyl 2-(2-(2-(4-((tert-butoxycarbonyl)amino)-3-fluorophenyl)thiazole-4-carboxamido)acrylamido)acrylate (30 mg, 59 μmol, 16%) as a white solid LCMS (22010199A TFA LCMS-5 Cl): RT: 1.815 min; Area: 98.1% (215 nm), 96.8% (306 nm); m/z=491.2 [M+H]⁺. ¹H NMR (400 MHz, CDCl₃) δ 9.97 (s, 1H), 8.55 (s, 1H), 8.23 (t, J=8.3 Hz, 1H), 8.12 (s, 1H), 7.81 (dd, J=11.7, 2.0 Hz, 1H), 7.68 (dd, J=8.6, 1.3 Hz, 1H), 6.86 (m, 1H), 6.77 (d, J=2.2 Hz, 1H), 6.72 (s, 1H), 6.04 (d, J=1.4 Hz, 1H), 5.51 (t, J=1.9 Hz, 1H), 3.90 (s, 3H), 1.57-1.49 (m, 9H).

### Example 26: Compound 71

### Methyl 2-(2-(2-(3-((tert-butoxycarbonyl)amino)phenyl)thiazole-4-carboxamido)acrylamido)acrylate

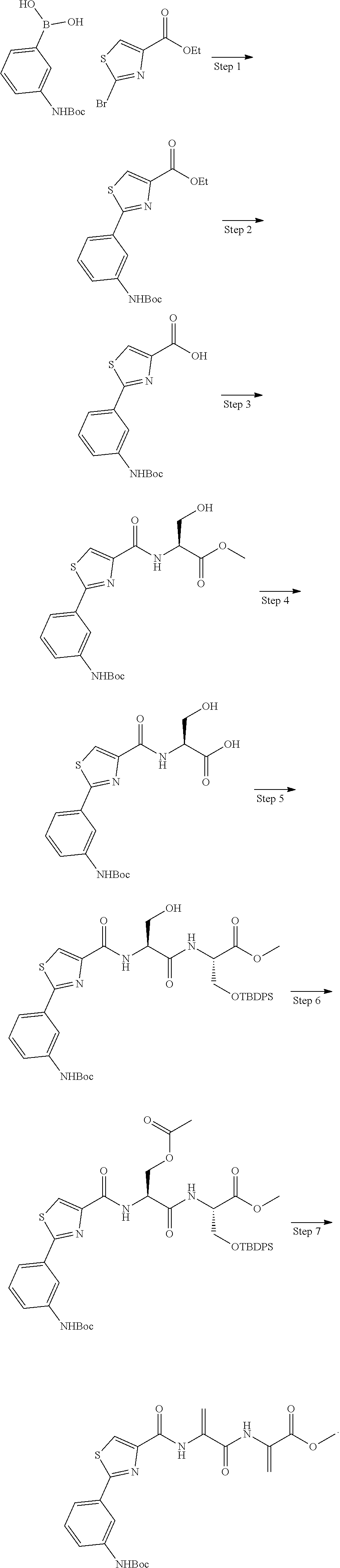

Step 1. Ethyl 2-(3-((tert-butoxycarbonyl)amino)phenyl)thiazole-4-carboxylate was prepared following General experimental procedure 1. Ethyl 2-bromothiazole-4-carboxylate (2.3 g, 1.05 eq., 9.7 mmol) and (3-((tert-butoxycarbonyl)amino)phenyl)boronic acid (2.2 g, 9.3 mmol) gave ethyl 2-(3-((tert-butoxycarbonyl)amino)phenyl)thi azole-4-carboxylate (1.4 g, 4.0 mmol, 430%) as a white solid. ¹H NMVR (400 MHUz, CDCk) 6 8.13 (s, 1H), 7.94 (t, J=2.0 Hz, 1H), 7.64 (ddd, J=7.7, 1.7, 1.0 Hz, 1H), 7.5 5 (d, J=8.2 Hz, 1H), 7.3 5 (t, J=8.0 Hz, 1H), 6.5 6 (s, 1H), 4.43 (q, J=7.1 Hz, 2H), 1.51 (s, 9H), 1.41 (t, J=7.1 Hz, 3H).

Step 2. 2-(3-((tert-butoxycarbonyl)amino)phenyl)thiazole-4-carboxylic acid was prepared following General experimental procedure 2. Ethyl 2-(3-((tert-butoxycarbonyl)amino)phenyl)thiazole-4-carboxylate (1.4 g, 4.0 mmol) gave 2-(3-((tert-butoxycarbonyl)amino)phenyl)thiazole-4-carboxylic acid (1.3 g, 3.8 mmol, 95%) as a white solid. ¹H NMR (400 MHz, CDCh) 6 8.22 (s, 1H), 8.05 (s, 1H), 7.59 (d, J=7.6 Hz, 1H), 7.46 (d, J=8.2 Hz, 1H), 7.37 (t, J=7.9 Hz, 1H), 1.53 (s, 9H).

Step 3. Methyl (2-(3-((tert-butoxycarbonyl)amino)phenyl)thiazole-4-carbonyl)serinate was prepared following General experimental procedure 4. 2-(3-((tert-butoxycarbonyl)amino)phenyl)thiazole-4-carboxylic acid (1.3 g, 3.8 mmol) and methyl L-serinate hydrochloride (0.65 g, 1.1 eq., 4.2 mmol) gave methyl (2-(3-((tert-butoxycarbonyl)amino)phenyl)thiazole-4-carbonyl)serinate (1.5 g, 3.5 mmol, 93%). ¹H NMR (299 MHz, CDCl₃) δ 8.27 (d, J=7.6 Hz, 1H), 6 8.14 (d, J=1.5 Hz, 1H), 7.98 (d, J=1.9 Hz, 1H), 7.61 (m, 2H), 7.41 (t, J=8.0 Hz, 1H), 7.28 (s, 5H), 6.66 (s, 1H), 5.00-4.84 (m, 1H), 4.12 (m, 2H), 3.87 (s, 3H), 1.57 (s, 9H).

Step 4. (2-(3-((tert-butoxycarbonyl)amino)phenyl)thiazole-4-carbonyl)serine was prepared following General experimental procedure 2. Methyl (2-(3-((tertbutoxy carbonyl)amino)phenyl)thiazole-4-carbonyl)serinate (1.5 g, 3.5 mmol) gave crude (2-(3-((tert-butoxycarbonyl)amino)phenyl)thiazole-4-carbonyl)serine (1.5 g, 3.8 mmol, quant) which was used as such in the next step.

Step 5. Methyl N-((2-(3-((tert-butoxycarbonyl)amino)phenyl)thiazole-4-carbonyl)-L-seryl)-O-(tert-butyldiphenylsilyl)-L-serinate was prepared following General experimental procedure 4. (2-(3-((tert-butoxycarbonyl)amino)phenyl)thiazole-4-carbonyl)serine (0.80 g, 2.0 mmol) and methyl O-(tert-butyldiphenylsilyl)-L-serinate hydrochloride (0.75 g, 1.9 mmol) gave methyl N-((2-(3-((tert-butoxycarbonyl)amino)phenyl)thiazole-4-carbonyl)-L-seryl)-O-(tert-butyldiphenylsilyl)-L-serinate (1.2 g, 1.6 mmol, 79%).

Step 6. Methyl N—(O-acetyl-N-(2-(3-((tert-butoxycarbonyl)amino)phenyl)thiazole-4-carbonyl)-L-seryl)-O-(tert-butyldiphenylsilyl)-L-serinate was prepared following General experimental procedure 5. Methyl N-((2-(3-((tert-butoxycarbonyl)amino)phenyl)thiazole-4-carbonyl)-L-seryl)-O-(tert-butyldiphenylsilyl)-L-serinate (1.2 g, 1.6 mmol) gave methyl N-(0-acetyl-N-(2-(3-((tert-butoxycarbonyl)amino)phenyl)thiazole-4-carbonyl)-L-seryl)-O-(tert-butyldiphenylsilyl)-L-serinate (1.1 g, 1.4 mmol, 89%).¹H NMR (400 MHz, CDC3) 6 8.11 (d, J=7.8 Hz, 1H), 8.07 (s, 1H), 7.90 (t, J=2.0 Hz, 1H), 7.62-7.50 (m, 6H), 7.44-7.26 (m, 8H), 7.13 (d, J=8.2 Hz, 1H), 6.57 (s, 1H), 4.92 (dt, J=7.9, 5.9 Hz, 1H), 4.67 (dt, J=8.1, 2.8 Hz, 1H), 4.51 (dd, J=11.3, 5.7 Hz, 1H), 4.39 (dd, J=11.3, 6.2 Hz, 1H), 4.11 (dd, J=10.3, 2.7 Hz, 1H), 3.87 (dd, J=10.3, 3.0 Hz, 1H), 3.73 (s, 3H), 2.08 (s, 3H), 1.52 (s, 9H), 0.96 (s, 9H).

Step 7. Methyl 2-(2-(2-(3-((tert-butoxycarbonyl)amino)phenyl)thiazole-4-carboxamido)acrylamido)acrylate was prepared following General experimental procedure 7. Methyl N—(O-acetyl-N-(2-(3-((tert-butoxycarbonyl)amino)phenyl)thiazole-4-carbonyl)-L-seryl)-O-(tert-butyldiphenylsilyl)-L-serinate (0.30 g, 0.38 mmol) gave methyl 2-(2-(2-(3-((tert-butoxycarbonyl)amino)phenyl)thiazole-4-carboxamido)acrylamido)acrylate (51 mg, 0.11 mmol, 57%) as a white solid. LCMS (22010199A TFA LCMS-5 Cl): RT: 1.704 min; Area: 98.9% (215 nm), 98.6% (306 nm); m/z 473.2 [M+H]⁺. xH NMR (400 MHz, CDCh) 6 10.00 (s, 1H), 8.55 (s, 1H), 8.15 (s, 1H), 7.93 (t, J=2.0 Hz, 1H), 7.67 (d, J=7.8 Hz, 1H), 7.62 (d, J=8.3 Hz, 1H), 7.40 (t, J=8.0 Hz, 1H), 6.78 (d, J=2.2 Hz, 1H), 6.71 (s, 1H), 6.66 (s, 1H), 6.03 (d, J=1.3 Hz, 1H), 5.51 (t, J=2.0 Hz, 1H), 3.91 (s, 3H), 1.55 (s, 9H).

### Example 27: Compound 72

### Methyl 2-(2-(2-(2-((tert-butoxycarbonyl)amino)pyrimidin-5-yl)thiazole-4-carboxamido)acrylamido)aerylate

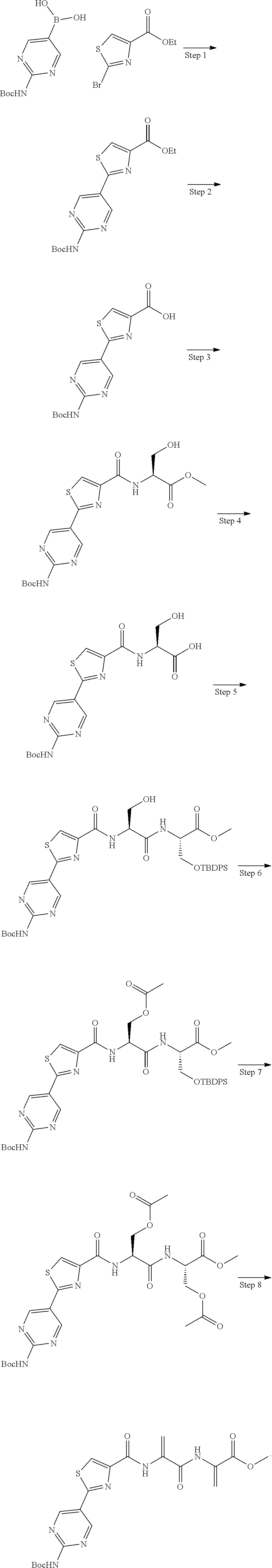

Step 1. Ethyl 2-(2-((tert-butoxycarbonyl)amino)pyrimidin-5-yl)thiazole-4-carboxylate was prepared following General experimental procedure 1. (2-((tert-butoxycarbonyl)amino)pyrimidin-5-yl)boronic acid (1.2 g, 5.2 mmol) and ethyl 2-bromothiazol e-4-carboxylate (1.4 g, 1.1 eq. 5.8 mmol) gave ethyl 2-(2-((tert-butoxycarbonyl)amino)pyrimidin-5-yl)thiazole-4-carboxylate (1.1 g, 3.1 mmol, 61%)¹H NMR (400 MHz, CDC3) 6 9.15 (s, 2H), 8.62 (s, 1H), 8.18 (s, 1H), 4.43 (q, J=7.1 Hz, 2H), 1.63 (s, 1H), 1.56 (s, 9H), 1.41 (t, J=7.1 Hz, 3H).

Step 2. 2-(2-((tert-butoxycarbonyl)amino)pyrimidin-5-yl)thiazole-4-carboxylic acid was prepared following General experimental procedure 2. Ethyl 2-(2-((tert-butoxycarbonyl)amino)pyrimidin-5-yl)thiazole-4-carboxylate (2.1 g, 5.9 mmol) gave crude 2-(2-((tert-butoxycarbonyl)amino)pyri mi din-5-yl)thiazole-4-carboxylic acid (2.0 g) which was used without purification in the next step.

Step 3. Methyl (2-(2-((tert-butoxycarbonyl)amino)pyrimidin-5-yl)thiazole-4-carbonyl)serinate was prepared following General experimental procedure 4. 2-(2-((tert-butoxycarbonyl)amino)pyrimidin-5-yl)thiazole-4-carboxylic acid (0.64 g, 2.0 mmol) and methyl L-serinate hydrochloride (0.34 g, 1.1 eq. 2.2 mmol) gave methyl (2-(2-((tert-butoxycarbonyl)amino)pyrimidin-5-yl)thiazole-4-carbonyl)serinate (0.93 g, 2.0 mmol, quant.).

Step 4. (2-(2-((tert-butoxycarbonyl)amino)pyrimidin-5-yl)thiazole-4-carbonyl)serine was prepared following General experimental procedure 2. Methyl (2-(2-((tert-butoxycarbonyl)amino)pyrimidin-5-yl)thiazole-4-carbonyl)serinate (0.84 g, 2.0 mmol) gave (2-(2-((tert-butoxycarbonyl)amino)pyrimidin-5-yl)thiazole-4-carbonyl)serine (0.55 g, 1.3 mmol, 67%). ¹H NMR (400 MHz, DMSO) δ 10.48 (s, 1H), 9.19 (s, 1H), 8.41 (s, 1H), 8.37 (d, J=8.1 Hz, 1H), 4.49 (dt, J=8.3, 4.2 Hz, 1H), 3.84 (m, 2H), 1.48 (s, 9H).

Step 5. Methyl N-((2-(2-((tert-butoxycarbonyl)amino)pyrimidin-5-yl)thiazole-4-carbonyl)-L-seryl)-O-(tert-butyldiphenylsilyl)-L-serinate was prepared following General experimental procedure 4. (2-(2-((tert-butoxycarbonyl)amino)pyrimidin-5-yl)thiazole-4-carbonyl)serine (0.54 g, 1.3 mmol) and methyl O-(tert-butyldiphenylsilyl)-L-serinate hydrochloride (0.57 g, 1.1 eq., 1.4 mmol) gave methyl N-((2-(2-((tert-butoxycarbonyl)amino)pyrimidin-5-yl)thiazole-4-carbonyl)-L-seryl)-O-(tert-butyldiphenylsilyl)-L-serinate (0.42 g, 0.56 mmol, 43%) as an off-white solid. LCMS (General 3 acidic) RT: 1.53 min; area % (254 nm): 76%; m/z=749.7 [M+H]⁺.

Step 6. Methyl N-(0-acetyl-N-(2-(2-((tert-butoxycarbonyl)amino)pyrimidin-5-yl)thiazole-4-carbonyl)-L-seryl)-O-(tert-butyldiphenylsilyl)-L-serinate was prepared following General experimental procedure 5. Methyl N-((2-(2-((tert-butoxycarbonyl)amino)pyrimidin-5-yl)thiazole-4-carbonyl)-L-seryl)-O-(tert-butyldiphenylsilyl)-L-serinate (0.40 g, 0.53 mmol) gave methyl N-(0-acetyl-N-(2-(2-((tert-butoxycarbonyl)amino)pyrimidin-5-yl)thiazole-4-carbonyl)-L-seryl)-O-(tert-butyldiphenylsilyl)-L-serinate (0.24 g, 0.30 mmol, 57%). LCMS (General 3 acidic) RT: 1.63 min; area % (254 nm): 96%; m z=691.5 [M-Boc+H]⁺.

Step 7. Methyl 0-acetyl-N-(0-acetyl-N-(2-(2-((tert-butoxycarbonyl)amino)pyrimidin-5-yl)thiazole-4-carbonyl)-L-seryl)-L-serinate was prepared using the following procedure. To a solution of methyl N-(0-acetyl-N-(2-(2-((tert-butoxycarbonyl)amino)pyrimidin-5-yl)thiazole-4-carbonyl)-L-seryl)-O-(tert-butyldiphenylsilyl)-L-serinate (0.24 g, 0.30 mmol) in THE (5 mL) was added a solution of TBAF in THE (1M, 0.45 mL, 1.5 eq., 0.45 mmol) and the resulting mixture was stirred for 1 hour. The mixture was concentrated and the residue taken up in MeCN (5 mL). Triethylamine (0.10 mL, 2.4 eq., 0.72 mmol) and acetic anhydride (38 μL, 1.3 eq., 0.40 mmol) were added and the resulting mixture was stirred at room temperature for 45 minutes. Water was added and the mixture was extracted with EtOAc. The organic layer was washed with brine, dried over Na₂SO₄, filtered, and concentrated. The crude material was purified by automated FCC to provide methyl O-acetyl-N—(O-acetyl-N-(2-(2-((tert-butoxycarbonyl)amino)pyrimidin-5-yl)thiazole-4-carbonyl)-L-seryl)-L-serinate (87 mg, 0.15 mmol, 48%).

Step 8. Methyl 2-(2-(2-(2-((tert-butoxycarbonyl)amino)pyrimidin-5-yl)thiazole-4-carboxamido)acrylamido)acrylate was prepared using the following procedure. To a solution of methyl O-acetyl-N-(0-acetyl-N-(2-(2-((tert-butoxycarbonyl)amino)pyrimidin-5-yl)thiazole-4-carbonyl)-L-seryl)-L-serinate (86 mg, 0.14 mmol) in CH₂CI2 (1.5 mL) and THE (1.5 mL) was added at 0° C. DBU (87 μL, 4 eq., 0.58 mmol) and the mixture was stirred at 0° C. for 2 hours. Water was added and the aqueous phase was adjusted to pH 4 by addition of 1M HCl. The mixture was extracted with CH₂CI2 and the organic layer was washed brine, dried over Na₂SO₄, filtered and concentrated. The crude material was purified by automated reverse phase FCC to provide methyl 2-(2-(2-(2-((tert-butoxycarbonyl)amino)pyrimidin-5-yl)thiazole-4-carboxamido)acrylamido)acrylate (27 mg, 57 μmol, 39%). LCMS (22010199A TFA LCMS-5 Cl): RT: 1.460 min; Area: 96.8% (215 nm), 96.2% (306 nm); m/z 473.0 [M−H]¹. H NMR (400 MHz, CDCh) 6 10.01 (s, 1H), 9.17 (s, 2H), 8.54 (s, 1H), 8.20 (s, 1H), 7.96 (s, 1H), 6.77 (d, J=2.3 Hz, 1H), 6.70 (s, 1H), 6.03 (d, J=1.3 Hz, 1H), 5.51 (t, J=1.9 Hz, 1H), 3.90 (s, 3H), 1.57 (s, 9H).

### Example 28: Compound 75

### Methyl 2-(2-(4-(4-((tert-butoxycarbonyl)amino)phenyl)thiazole-2-carboxamido)acrylamido)aciylate

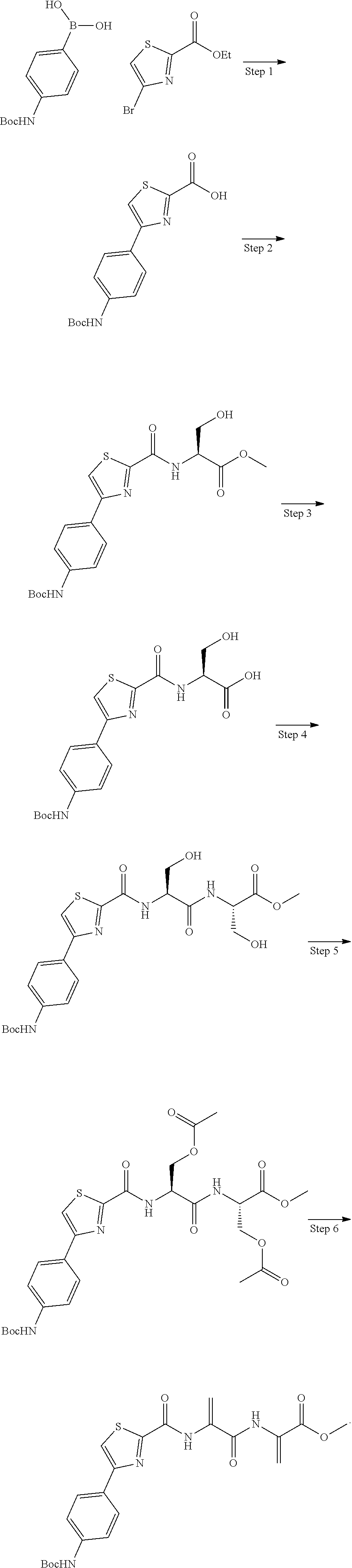

Step 1. 4-(4-((tert-butoxycarbonyl)amino)phenyl)thiazole-2-carboxylic acid was prepared using the following procedure. To a solution of ethyl 4-bromothiazole-2-carboxylate (1.0 g, 4.2 mmol) and (4-((tert-butoxycarbonyl)amino)phenyl)boronic acid in 1,2-Dimethoxy ethane (25 mL) were added sodium carbonate (aq. 2 M, 10 eq. 42 mmol) and palladium tetrakis (0.24 g, 0.05 eq., 0.21 mmol) and the resulting mixture was stirred at 80° C. for 16 hours. The mixture was cooled to room temperature, diluted with ethyl acetate and washed with water. The aqueous phase was acidified with 1M HCl solution and extracted with ethyl acetate. The organic layer was dried over Na₂SO₄, filtered, and concentrated under reduced pressure to afford 4-(4-((tert-butoxycarbonyl)amino)phenyl)thiazole-2-carboxylic acid (1.1 g, purity 43%, 1.4 mmol, 35%) as a mixture with the decarboxylated product. The mixture was used as such in the next step.

Step 2. Methyl (4-(4-((tert-butoxycarbonyl)amino)phenyl)thiazole-2-carbonyl)-L-serinate was prepared following General experimental procedure 4. Crude 4-(4-((tert-butoxycarbonyl)amino)phenyl)thiazole-2-carboxylic acid (1.1 g, purity 43%, 1.4 mmol) and methyl L-serinate hydrochloride (0.26 g, 1.2 eq. 1.7 mmol) gave methyl (4-(4-((tert-butoxycarbonyl)amino)phenyl)thiazole-2-carbonyl)-L-serinate (0.61 g 1.45 mmol, quant.).

Step 3. (4-(4-((tert-butoxycarbonyl)amino)phenyl)thiazole-2-carbonyl)-L-serine was prepared following General experimental procedure 2. Methyl (4-(4-((tert-butoxycarbonyl)amino)phenyl)thiazole-2-carbonyl)-L-serinate (0.60 g, 1.4 mmol) gave (4-(4-((tert-butoxycarbonyl)amino)phenyl)thiazole-2-carbonyl)-L-serine (0.59 g, 1.4 mmol, quant).

Step 4. Methyl (4-(4-((tert-butoxycarbonyl)amino)phenyl)thiazole-2-carbonyl)-L-seryl-L-serinate was prepared following General experimental procedure 4. (4-(4-((tert-butoxycarbonyl)amino)phenyl)thiazole-2-carbonyl)-L-serine (0.58 g, 1.4 mmol) and methyl L-serinate hydrochloride (0.27 g, 1.2 eq., 1.7 mmol) gave methyl (4-(4-((tert-butoxycarbonyl)amino)phenyl)thiazole-2-carbonyl)-L-seryl-L-serinate (0.52 g, 1.0 mmol, 72%). LCMS (General 3 acidic) RT: 0.94 min; area % (254 nm): 91%; m/z=509.4 [M+H]⁺.

Step 5. Methyl O-acetyl-N—(O-acetyl-N-(4-(4-((tert-butoxycarbonyl)amino)phenyl)thiazole-2-carbonyl)-L-seryl)-L-serinate was prepared following General experimental procedure 5. Methyl (4-(4-((tert-butoxycarbonyl)amino)phenyl)thiazole-2-carbonyl)-L-seryl-L-serinate (0.52 g, 1.0 mmol) and acetic anhydride (0.25 mL, 4 eq., 2.7 mmol) gave methyl O-acetyl-N—(O-acetyl-N-(4-(4-((tert-butoxycarbonyl)amino)phenyl)thiazole-2-carbonyl)-L-seryl)-L-serinate (0.33 g, 0.56 mmol, 55%)

Step 6. Methyl 2-(2-(4-(4-((tert-butoxycarbonyl)amino)phenyl)thiazole-2-carboxamido)acrylamido)acrylate was prepared following General experimental procedure 8. Methyl O-acetyl-N—(O-acetyl-N-(4-(4-((tert-butoxycarbonyl)amino)phenyl)thiazole-2-carbonyl)-L-seryl)-L-serinate (0.35 g, 0.59 mmol) gave methyl 2-(2-(4-(4-((tert-butoxycarbonyl)amino)phenyl)thiazole-2-carboxamido)acrylamido)acrylate (85 mg, 0.18 mmol, 30%) as a white solid. LC-MS (22010199A TFA LCMS-5 Cl): RT: 1.731 min; Area 98.6% (215 nm), 97.8% (304 nm); m z=473.2 [M+H]⁺. ¹H NMR (400 MHz, CDCh) 6 (ppm) 9.92 (s, 1H), 8.55 (s, 1H), 7.89 (d, J=8.7 Hz, 2H), 7.67 (s, 1H), 7.45 (d, J=8.6 Hz, 2H), 6.76 (d, J=2.4 Hz, 1H), 6.71 (s, 1H), 6.59 (s, 1H), 6.04 (d, J=1.0 Hz, 1H), 5.54 (dd, J=2.3 Hz, J=1.6 Hz, 1H), 3.90 (s, 3H), 1.54 (s, 9H).

### Example 29: Compound 76

### Methyl 2-(2-(2-(4-((tert-butoxycarbonyl)amino)-2-chlorophenyl)thiazole-4-carboxamido)acrylamido)acrylate

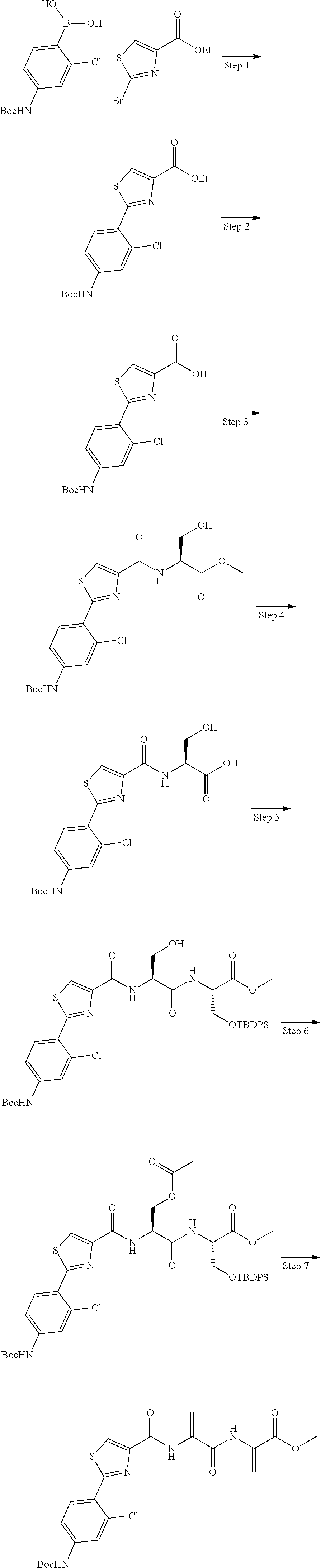

Step 1. Ethyl 2-4(tert-butoxycarbonyl)amino-2-chlorophenyl)thiazole-4-carboxylate was prepared following General experimental procedure 1. Ethyl 2-bromothiazole-4-carboxylate (0.47 g, 2.0 mmol) and tert-butyl (3-chloro-4-(4,4,5,5-tetramethyl-1,3,2,-dioxaborolan-2-yl)phenyl)carbamate (0.70 g, 2.0 mmol) gave ethyl 2-(4-((tert-butoxycarbonyl)amino)-2-chlorophenyl)thiazole-4-carboxylate (0.38 g, 1.0 mmol, 51%). LCMS (General 3 acidic) RT: 1.58 min; area% (254 nm): 96%; m/z=383.2 [M+H]⁺.

Step 2. 2-(4-((tert-butoxycarbonyl)amino)-2-chlorophenyl)thiazole-4-carboxylic acid was prepared following General experimental procedure 2. Ethyl 2-(4-((tert-butoxycarbonyl)amino-2chlorphenyl)thiazole-4-carboxylate (0.38 g, 0.99 mmol) gave 2-(4-((tert-butoxycarbonyl)amino)-2-chlorophenyl)thiazole-4-carboxylic acid (0.30 g, 0.84 mmol, 85%) ¹H NMR (299 MHz, CDCk) 6 8.36 (s, 1H), δ8.25 (d, J=8.9 Hz, 1H), 7.82 (s, 1H), 7.31, 7.22 (m, 2H), 6.78 (s, 1H), 1.56 (s, 9H).

Step 3. Methyl (2-(4-((tert-butoxycarbonyl)amino)-2-chlorophenyl)thiazole-4-carbonyl)-L-serinate was prepared following General experimental procedure 4. 2-(4-((tert-butoxycarbonyl)amino)-2-chlorophenyl)thiazole-4-carboxylic acid (0.44 g, 1.2 mmol) and methyl L-serinate hydrochloride (0.23 g, 1.2 eq., 1.5 mmol) gave methyl (2-(4-((tert-butoxycarbonyl)amino)-2-chlorophenyl)thiazole-4-carbonyl)-L-serinate (0.42 g, 0.92 mmol, 75%) %). LCMS (General 3 acidic) RT: 1.23 min; area % (254 nm): 93%; m z=456.3 [M+H]⁺.

Step 4. (2-(4-((tert-butoxycarbonyl)amino)-2-chlorophenyl)thiazole-4-carbonyl)-L-serine was prepared following. General experimental procedure 2. Methyl (2-(4-((tert-butoxycarbonyl)amino)-2-chlorophenyl)thiazole-4-carbonyl)-L-serinate (0.42 g, 0.92 mmol) gave (2-(4-((tert-butoxycarbonyl)amino)-2-chlorophenyl)thiazole-4-carbonyl)-L-serine (0.33 g, 0.75 mmol, 81%) as an orange solid.

Step 5. Methyl N-((2-(4-((tert-butoxycarbonyl)amino)-2-chlorophenyl)thiazole-4-carbonyl)-L-seryl)-O-(tert-butyldiphenylsilyl)-L-serinate was prepared following General experimental procedure 4. (2-(4-((tert-butoxycarbonyl)amino)-2-chlorophenyl)thiazole-4-carbonyl)-L-serine (0.33 g, 0.75 mmol) and methyl O-(tert-butyl diphenyl silyl)-L-serinate hydrochloride (0.35 g, 1.2 eq., 0.90 mmol) gave methyl N-((2-(4-((tert-butoxycarbonyl)amino)-2-chlorophenyl)thiazole-4-carbonyl)-L-seryl)-O-(tert-butyldiphenylsilyl)-L-serinate (0.37 g, 0.48 mmol, 64%).

Step 6. Methyl N—(O-acetyl-N-(2-(4-((tert-butoxycarbonyl)amino)-2-chlorophenyl)thiazole-4-carbonyl)-L-seryl)-O-(tert-butyldiphenylsilyl)-L-serinate was prepared following General experimental procedure 5. Methyl N-((2-(4-((tert-butoxycarbonyl)amino)-2-chlorophenyl)thiazole-4-carbonyl)-L-seryl)-O-(tert-butyldiphenylsilyl)-L-serinate (0.37 g, 0.48 mmol) and acetic anhydride (68 μL, 1.5 eq. 0.72 mmol) gave methyl N—(O-acetyl-N-(2-(4-((tert-butoxycarbonyl)amino)-2-chlorophenyl)thiazole-4-carbonyl)-L-seryl)-O-(tert-butyldiphenylsilyl)-L-serinate (0.22 g, 0.27 mmol, 57%).¹H NMR (299 MHz, CDC3) 6 8.28-8.12 (m, 3H), 7.85 (s, 1H), 7.57 (m, 4H), 7.46-7.30 (m, 6H), 7.19 (m, 2H), 6.66 (s, 1H), 4.95 (q, J=6.3 Hz, 1H), 4.70 (d, J=8.1 Hz, 1H), 4.56 (dd, J=11.3, 5.7 Hz, 1H), 4.43 (dd, J=11.3, 6.2 Hz, 1H), 4.14 (d, J=8.1 Hz, 1H), 3.96-3.86 (m, 1H), 3.77 (d, J=1.2 Hz, 3H), 2.11 (s, 3H), 1.56 (s, 9H), 1.00 (s, 9H).

Step 7. Methyl 2-(2-(2-(4-((tert-butoxycarbonyl)amino)-2-chlorophenyl)thiazole-4-carboxamido)acrylamido)acrylate was prepared following General experimental procedure 7. Methyl N—(O-acetyl-N-(2-(4-((tert-butoxycarbonyl)amino)-2-chlorophenyl)thiazole-4-carbonyl)-L-seryl)-O-(tert-butyldiphenylsilyl)-L-serinate (0.22 g, 0.27 mmol) gave methyl 2-(2-(2-(4-((tert-butoxycarbonyl)amino)-2-chlorophenyl)thiazole-4-carboxamido)acrylamido)acrylate (15 mg, 30 μmol, 29%) as a white solid. LCMS (22010199A TFA LCMS-5 Cl): rt 1.880 min; Area 96.5% (215 nm), 95.6% (304 nm); m z=507.2 [M+H]⁺.¹H NMR (400 MHz, CDCl₃) δ (ppm) 10.02 (s, 1H), 8.55 (s, 1H), 8.31 (d, J=8.7 Hz, 1H), 8.23 (s, 1H), 7.84 (d, J=1.8 Hz, 1H), 7.21 (dd, J=8.7 Hz, J=2.3 Hz, 1H), 6.78 (d, J=2.2 Hz, 1H), 6.70 (s, 1H), 6.66 (s, 1H), 6.02 (d, J=1.1 Hz, 1H), 5.48 (t, J=1.8 Hz, 1H), 3.90 (s, 3H), 1.54 (s, 9H).

### Example 30: Compound 77

### Methyl 2-(2-(4′-((tert-butoxycarbonyl)amino)-[1,1′-biphenyl]-3-carboxamido)acrylamido)acrylate

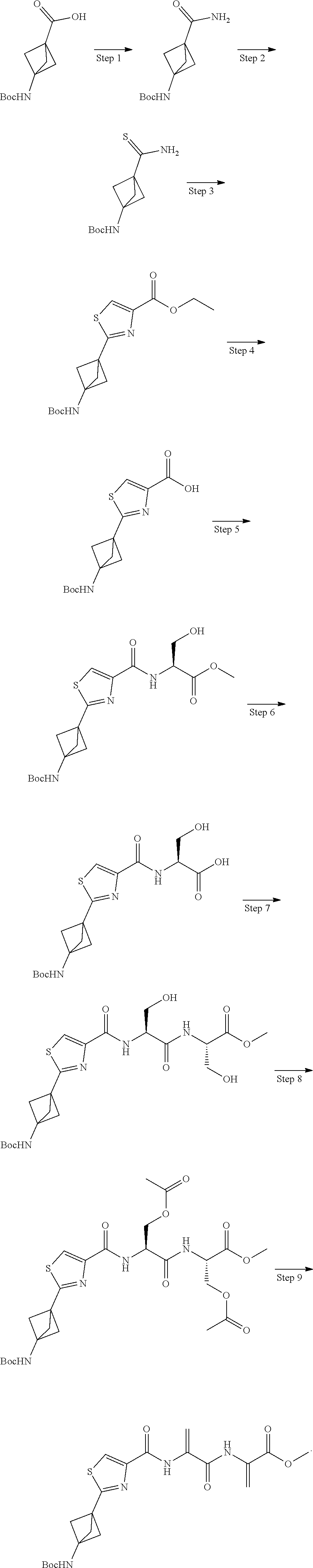

Step 1. 4′((tertbutoxycarbonyl)amino)-[1,1′biphenyl]-3-carboxylic acid was prepared using the following procedure. To a solution of methyl 4′-amino-[1,1′-biphenyl]-3-carboxylate (0.50 g, 2.2 mmol) in CH₂CI2 (20 mL) was added DMAP (0.54 g, 2 eq., 4.4 mmol). Then BOC2O (0.72 g, 1.5 eq., 3.3 mmol) was added portionwise and the mixture was stirred over 16 hours at room temperature. The mixture was washed with water and brine, dried over Na₂SO₄ and concentrated in vacuo. The crude material was dissolved in THE (20 mL) and a solution of NaOH (aq., 1M, 7eq. 15.4 mL, 15 mmol) was added and the resulting mixture was stirred for 64 hours. The mixture was quenched with 1M HCl, extracted with EtOAc. The organic phase was dried over Na₂SO₄, filtered, and concentrated in vacuo to provide 4′-((tert-butoxycarbonyl)amino)-[1,1′-biphenyl]-3-carboxylic acid (0.47 g, 1.5 mmol, 68%) as a yellow solid.

Step 2. Methyl (4′-((tert-butoxycarbonyl)amino)-[1,T-biphenyl]-3-carbonyl)-L-serinate was prepared following General experimental procedure 4. 4′-((tert-butoxycarbonyl)amino)-[1,1′-biphenyl]-3-carboxylic acid (0.45 g, 1.4 mmol) and methyl L-serinate hydrochloride (0.27 g, 1.2 eq. 1.7 mmol) gave crude methyl (4′-((tert-butoxycarbonyl)amino)-[1,T-biphenyl]-3-carbonyl)-L-serinate (0.60 g) which was used as such in the next step.

Step 3. (4′-((tert-butoxycarbonyl)amino)-[1,1′-biphenyl]-3-carbonyl)-L-serine was prepared following General experimental procedure 2. Methyl (4′-((tert-butoxycarbonyl)amino)-[1,1′-biphenyl]-3-carbonyl)-L-serinate (0.58 g, 1.4 mmol) gave (4′-((tert-butoxycarbonyl)amino)-[1,1′-biphenyl]-3-carbonyl)-L-serine (0.37 g, 0.92 mmol, 66% ver 2 steps) as a yellow oil. LCMS (General 3 acidic) RT: 0.97 min; area % (254 nm): 90%; m/z=401.3 [M+H]⁺.

Step 4. Methyl (4′-((tert-butoxycarbonyl)amino)-[1,1′-biphenyl]-3-carbonyl)-L-seryl-L-serinate was prepared following General experimental procedure 4. (4′-((tert-butoxycarbonyl)amino)-[1,1′-biphenyl]-3-carbonyl)-L-serine (0.20 g, 0.50 mmol) and methyl L-serinate hydrochloride (0.93 mg, 1.2 eq. 0.60 mmol) gave crude methyl (4′-((tert-butoxycarbonyl)amino)-[1,1′-biphenyl]-3-carbonyl)-L-seryl-L-serinate (0.16 g) which was used as such in the next step. LCMS (General 3 acidic) RT: 0.95 min; area % (254 nm): 84%; m z=502.4 [M+H]⁺.

Step 5. Methyl O-acetyl-N—(O-acetyl-N-(4′-((tert-butoxycarbonyl)amino)-[1,1′-biphenyl]-3-carbonyl)-L-seryl)-L-serinate was prepared following General experimental procedure 5. Crude methyl (4′-((tert-butoxycarbonyl)amino)-[1,1′-biphenyl]-3-carbonyl)-L-seryl-L-serinate (0.16 g) and acetic anhydride (0.12 mL, 2.6 eq. 1.3 mmol) gave methyl O-acetyl-N—(O-acetyl-N-(4′-((tert-butoxycarbonyl)amino)-[1,1′-biphenyl]-3-carbonyl)-L-seryl)-L-serinate (130 mg, 0.22 mmol, 44%) and mono-eliminated product methyl O-acetyl-N-(2-(4′-((tert-butoxycarbonyl)amino)-[1,1′-biphenyl]-3-carboxamido)acryloyl)-L-serinate (0.10 g, 0.19 mmol, 38%)

Step 6. Methyl 2-(2-(4′-((tert-butoxycarbonyl)amino)-[1,l′-biphenyl]-3-carboxamido)acrylamido)acrylate was prepared following General experimental procedure 8. A mixture of methyl O-acetyl-N—(O-acetyl-N-(4′-((tert-butoxycarbonyl)amino)-[1,1′-biphenyl]-3-carbonyl)-L-seryl)-L-serinate (130 mg, 0.22 mmol) and methyl O-acetyl-N-(2-(4′-((tert-butoxycarbonyl)amino)-[1,1′-biphenyl]-3-carboxamido)acryloyl)-L-serinate (0.10 g, 0.19 mmol) as prepared in step 5 gave methyl 2-(2-(4′-((tert-butoxycarbonyl)amino)-[l,r-biphenyl]-3-carboxamido)acrylamido)acrylate (6.6 mg, 14 μmol) as a white solid. LCMS (22010199A TFA LCMS-5 Cl): RT:1.468 min; Area 98.0% (215 nm), 97.2% (304 nm); m/z=466.2 [M+H]⁺.¹H NMR (400 MHz, CDCl₃) δ 8.95 (s, 1H), 8.61 (s, 1H), 8.08 (t, J=1.9 Hz, 1H), 7.78 (ddt, J=15.4, 7.9, 1.3 Hz, 2H), 7.62-7.45 (m, 6H), 6.85 (d, J=2.3 Hz, 1H), 6.68 (s, 1H), 6.60 (s, 1H), 6.05 (d, J=1.4 Hz, 1H), 5.53 (t, J=1.8 Hz, 1H), 3.93 (s, 3H), 1.56 (s, 9H).

### Example 31: Compound 78

### methyl 2-(2-(2-(3-((tert-butoxycarbonyl)amino)bicyclo[1.1.1]pentan-1-yl)thiazole-4-carboxamido)acrylamido)acrylate

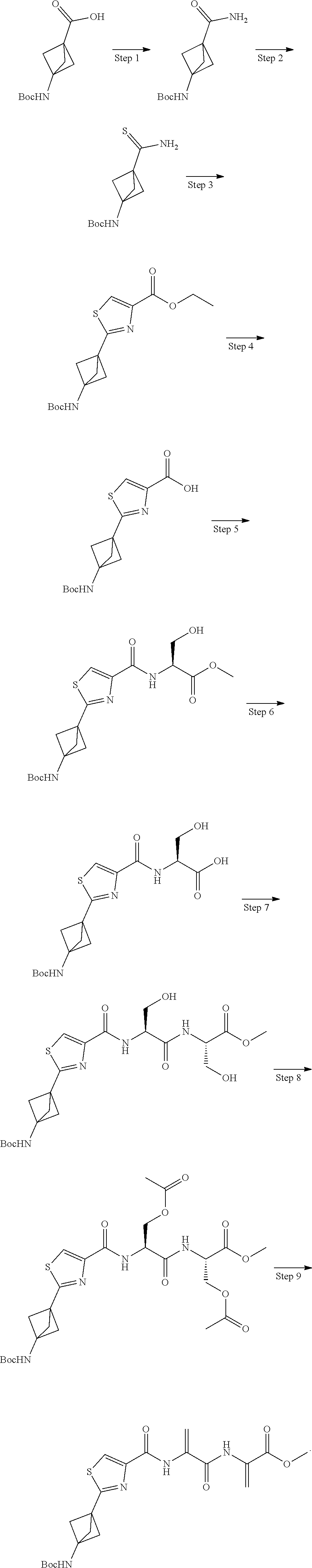

Step 1. tert-butyl (3-carbamoylbicyclo[1.1.1]pentan-1-yl)carbamate was prepared following General experimental procedure 9.3-((tert-butoxycarbonyl)amino)bicyclo[1.1.1]pentane-1-carboxylic acid (2.0 g, 8.8 mmol) gave tert-butyl (3-carbarn oylbicyclof 1.1.1]pentan-1-yl)carbamate (1.8 g, 8.1 mmol, 92% ) as a white solid.

Step 2. tert-butyl (3-carbamothioylbicyclo[1.1. 1]pentan-1-yl)carbamate was prepared following General experimental procedure 10. tert-butyl (3-carbamoylbicyclo[1.1.1]pentan-1-yl)carbamate (1.8 g, 8.1 mmol) gave tert-butyl (3-carbarnothioylbicyclo[1.1.1]pentan-1-yl)carbamate (1.5 g, 6.4 mmol, 78%) LCMS (General 3 acidic) RT: 0.79 min; area % (254 nm): 100%

Step 3. Ethyl 2-(3-((tert-butoxycarbonyl)amino)bicyclo[1.1.1]pentan-1-yl)thiazole-4-carboxylate was prepared following General experimental procedure 11. tert-butyl (3-carbamothioylbicyclo[1.1.1]pentan-1-yl)carbamate (1.5 g, 6.1 mmol) gave ethyl 2-(3-((tert-butoxycarbonyl)amino)bicyclo[1.1.1]pentan-1-yl)thiazole-4-carboxylate (1.8 g, 5.4 mmol, 88%). ¹H NMR (400 MHz, CDCl₃) δ 8.06 (s, 1H), 5.00 (s, 1H), 4.41 (q, J=7.1 Hz, 2H), 2.49 (s, 6H), 1.46 (s, 9H), 1.39 (t, J=7.1 Hz, 3H).

Step 4. 2-(3-((tert-butoxycarbonyl)amino)bicyclo[1.l. 1]pentan-1-yl)thiazole-4-carboxylic acid was prepared following General experimental procedure 2. Ethyl 2-(3-((tert-butoxycarbonyl)amino)bicyclo[1.1.1]pentan-1-yl)thiazole-4-carboxylate (1.8 g, 5.4 mmol) gave 2-(3-((tert-butoxycarbonyl)amino)bicyclo[1.1.1]pentan-1-yl)thiazole-4-carboxylic acid (1.7 g, 5.4 mmol, quant.)

Step 5. Methyl (2-(3-((tert-butoxycarbonyl)amino)bicyclo[1.1.1]pentan-1-yl)thiazole-4-carbonyl)-L-serinate was prepared following General experimental procedure 4. 2-(3-((tert-butoxycarbonyl)amino)bicyclo[1.1.1]pentan-1-yl)thiazole-4-carboxylic acid (1.7 g, 5.4 mmol) and methyl L-serinate hydrochloride (0.92 g, 1.1 eq., 5.9 mmol) gave methyl (2-(3-((tert-butoxycarbonyl)amino)bicyclo[1.1.1]pentan-1-yl)thiazole-4-carbonyl)-L-serinate (1.6 g, 90% purity, 3.6 mmol, 67%) as a white solid. ¹H NMR (400 MHz, CDCl₃) δ 8.07 (d, J=7.7 Hz, 1H), 8.00 (s, 1H), 4.83 (m, 1H), 4.13-3.98 (m, 2H), 3.81 (s, 3H), 2.44 (s, 6H), 1.45 (s, 9H).

Step 6. (2-(3-((tert-butoxycarbonyl)amino)bicyclo[1.1.1]pentan-1-yl)thiazole-4-carbonyl)-L-serine was prepared following General experimental procedure 2. Methyl (2-(3-((tert-butoxycarbonyl)amino)bicyclo[1.1.1]pentan-1-yl)thiazole-4-carbonyl)-L-serinate (1.6 g, 90% purity, 3.6 mmol) gave (2-(3-((tert-butoxycarbonyl)amino)bicyclo[1.1.1]pentan-1-yl)thiazole-4-carbonyl)-L-serine (1.5 g, 85% purity, 3.2 mmol, 89%)

Step 7. Methyl (2-(3-((tert-butoxycarbonyl)amino)bicyclo[1.1.1]pentan-1-yl)thiazole-4-carbonyl)-L-seryl-L-serinate was prepared following General experimental procedure 4. (2-(3-((tert-butoxycarbonyl)amino)bicyclo[1.1.1]pentan-1-yl)thiazole-4-carbonyl)-L-serine (1.5 g, 3.2 mmol) and methyl L-serinate hydrochloride (0.70 g, 1.4 eq., 4.5 mmol) gave methyl (2-(3-((tert-butoxycarbonyl)amino)bicyclo[1.1.1]pentan-1-yl)thiazole-4-carbonyl)-L-seryl-L-serinate (1.6 g, 3.2 mmol, 98%)

Step 8. Methyl O-acetyl-N—(O-acetyl-N-(2-(3-((tert-butoxycarbonyl)amino)bicyclo[1.1.1]pentan-1-yl)thiazole-4-carbonyl)-L-seryl)-L-serinate was prepared following General experimental procedure 5. Methyl (2-(3-((tert-butoxycarbonyl)amino)bicyclo[1.1.1]pentan-1-yl)thiazole-4-carbonyl)-L-seryl-L-serinate (1.6 g, 3.2 mmol) gave methyl O-acetyl-N—(O-acetyl-N-(2-(3-((tert-butoxycarbonyl)amino)bicyclo[1.1.1]pentan-1-yl)thiazole-4-carbonyl)-L-seryl)-L-serinate (1.2 g, 2.1 mmol, 68%) as a white solid.

Step 9. Methyl 2-(2-(2-(3-((tert-butoxycarbonyl)amino)bicyclo[1.1.1]pentan-1-yl)thiazole-4-carboxamido)acrylamido)acrylate was prepared following General experimental procedure 8. Methyl O-acetyl-N—(O-acetyl-N-(2-(3-((tertbutoxy carbonyl)amino)bicyclo[1.1.1]pentan-1-yl)thiazole-4-carbonyl)-L-seryl)-L-serinate (0.17 g, 0.32 mmol) gave methyl 2-(2-(2-(3-((tertbutoxy carbonyl)amino)bicyclo[1.1.1]pentan-1-yl)thiazole-4-carboxamido)acrylamido)acrylate (85 mg, 0.18 mmol, 58%) as a white solid. LCMS (22010199A TFA LCMS-5 Cl): rt 1.319 min; Area 99.0% (215 nm), 98.7% (304 nm); m z=463.2[M+H]⁺.¹H NMR (400 MHz, CDCl₃) δ 9.81 (s, 1H), 8.51 (s, 1H), 8.05 (s, 1H), 6.73 (d, J=2.1 Hz, 1H), 6.69 (s, 1H), 6.01 (d, J=1.3 Hz, 1H), 5.47 (t, J=1.9 Hz, 1H), 5.02 (s, 1H), 3.90 (s, 3H), 2.48 (s, 6H), 1.47 (s, 9H).

### Example 32: Compound 79

### methyl 2-(2-(2-(4-((tert-butoxycarbonyl)amino)cyclohexyl)thiazole-4-carboxamido)acrylamido)acrylate

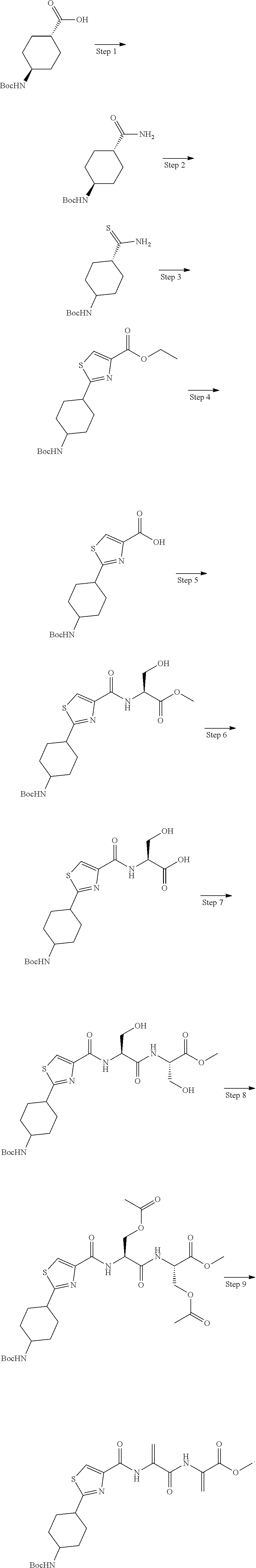

Step 1. tert-butyl (traw-4-carbamoylcyclohexyl)carbamate was prepared following General experimental procedure 9. Trans-4-((tert-butoxycarbonyl)amino)cyclohexane-1-carboxylic acid (2.0 g, 8.2 mmol) gave tert-butyl (trans-4-carbamoylcyclohexyl)carbamate (1.9 g, 7.7 mmol, 94%) as a white solid.

Step 2. tert-butyl (4-carbamothioylcyclohexyl)carbamate was prepared following General experimental procedure 10. tert-butyl (trans-4-carbamoylcyclohexyl)carbamate (1.9 g, 7.7 mmol) gave tert-butyl (4-carbamothioylcyclohexyl)carbamate (1.2 g, 4.7 mmol, 61% , trans/cis 97:3) as an off-white solid.

Step 3. Ethyl 2-(4-((tert-butoxycarbonyl)amino)cyclohexyl)thiazole-4-carboxylate was prepared following General experimental procedure 11. tert-butyl (4-carbamothioylcyclohexyl)carbamate (1.2 g, 4.7 mmol) gave ethyl 2-(4-((tert-butoxycarbonyl)amino)cyclohexyl)thiazole-4-carboxylate (1.1 g, 3.0 mmol, 6400, trans/cis 9:1).

Step 4. 2-(4-((tert-butoxycarbonyl)amino)cyclohexyl)thiazole-4-carboxylic acid was prepared following General experimental procedure 2. Ethyl 2-(4-((tert-butoxycarbonyl)amino)cyclohexyl)thiazole-4-carboxylate (1.1 g, 3.0 mmol) gave 2-(4-((tert-butoxycarbonyl)amino)cyclohexyl)thiazole-4-carboxylic acid (0.97 g, 3.0 mmol, 99%, trans cis 9:1)

Step 5. Methyl (2-(4-((tert-butoxycarbonyl)amino)cyclohexyl)thiazole-4-carbonyl)-L-serinate was prepared following General experimental procedure 4. 2-(4-((tert-butoxycarbonyl)amino)cyclohexyl)thiazole-4-carboxylic acid (0.97 g, 3.0 mmol) and methyl L-serinate hydrochloride (0.51 g, 1.1 eq., 3.3 mmol) gave methyl (2-(4-((tert-butoxycarbonyl)amino)cyclohexyl)thiazole-4-carbonyl)-L-serinate (1.1 g, 89% purity, 2.2 mmol, 75%)

Step 6. (2-(4-((tert-butoxycarbonyl)amino)cyclohexyl)thiazole-4-carbonyl)-L-serine was prepared following General experimental procedure 2. Methyl (2-(4-((tert-butoxycarbonyl)amino)cyclohexyl)thiazole-4-carbonyl)-L-serinate (1.1 g, 89% purity, 2.2 mmol) gave (2-(4-((tert-butoxycarbonyl)amino)cyclohexyl)thiazole-4-carbonyl)-L-serine (1.0 g, 88% purity, 2.1 mmol, 97%).

Step 7. Methyl (2-(4-((tert-butoxycarbonyl)amino)cyclohexyl)thiazole-4-carbonyl)-L-seryl-L-serinate was prepared following General experimental procedure 4. (2-(4-((tert-butoxycarbonyl)amino)cyclohexyl)thiazole-4-carbonyl)-L-serine (1.0 g, 88% purity, 2.1 mmol) and methyl L-serinate hydrochloride (0.36 g, 1.1 eq., 2.3 mmol) gave methyl (2-(4-((tert-butoxycarbonyl)amino)cyclohexyl)thiazole-4-carbonyl)-L-seryl-L-serinate (0.55 g, 90% purity, 0.96 mmol, 45%).

Step 8. Methyl O-acetyl-N—(O-acetyl-N-(2-(4-((tert-butoxycarbonyl)amino)cyclohexyl)thiazole-4-carbonyl)-L-seryl)-L-serinate was prepared following General experimental procedure 5. Methyl (2-(4-((tert-butoxycarbonyl)amino)cyclohexyl)thiazole-4-carbonyl)-L-seryl-L-serinate (0.55 g, 90% purity, 0.96 mmol) gave methyl O-acetyl-N—(O-acetyl-N-(2-(4-((tert-butoxycarbonyl)amino)cyclohexyl)thiazole-4-carbonyl)-L-seryl)-L-serinate (0.46 g, 0.77 mmol, 80%, trans/cis 87:13) Step 9. Methyl 2-(2-(2-(4-((tert-butoxycarbonyl)amino)cyclohexyl)thiazole-4-carboxamido)acrylamido)acrylate was prepared following General experimental procedure 8. Methyl O-acetyl-N—(O-acetyl-N-(2-(4-((tert-butoxycarbonyl)amino)cyclohexyl)thiazole-4-carbonyl)-L-seryl)-L-serinate (0.20 g, 0.33 mmol) gave two batches of methyl 2-(2-(2-(4-((tert-butoxycarbonyl)amino)cyclohexyl)thiazole-4-carboxamido)acrylamido)acrylate 79-1 and 79-2.

79-1 (81 mg, 0.17 mmol, 51%, trans cis 89: 11) LCMS (22010199A TFA LCMS-5 Cl): RT: 1.492 min; Area: 86.2% (215 nm), 88.4% (306 nm); m/z 479.2 (M−H+); RT: 1.520 min; Area: 10.80% (215 nm), 11.61% (306 nm); m/z=479.2 [M+H]⁺¹H-NMR (major isomer) (400 MHz, CDCl₃) δ 9.90 (s, 1H), 8.53 (s, 1H), 8.03 (s, 1H), 6.74 (d, J=2.2 Hz, 1H), 6.70 (s, 1H), 6.01 (d, J=1.3 Hz, 1H), 5.46 (t, J=1.9 Hz, 1H), 4.43 (s, 1H), 3.90 (s, 3H), 3.51 (s, 1H), 2.97 (tt, J=12.1, 3.6 Hz, 1H), 2.25 (d, J=13.4 Hz, 2H), 2.17 (d, =12.7 Hz, 2H), 1.66 (qd, J=13.1, 3.2 Hz, 2H), 1.46 (s, 9H), 1.27 (qd, J=12.7, 3.4 Hz, 2H).

79-2 14 mg, 29 μmol, 8.7%, trans/cis 63:37. LCMS-5 (22010199A TFA LCMS-5 Cl): RT: 1.493 min; Area: 55.3% (215 nm), 54.5% (306 nm); m/z 479.2 (M−H+); RT: 1.526 min; Area: 42.9% (215 nm), 43.6% (306 nm); m z=379.2 [M-Boc+H]⁺. ¹H NMR (400 MHz, CDCl₃) δ 9.99 (s, OH), 9.90 (s, 1H), 8.54 (m, 1H), 8.03 (m, 1H), 6.74 (d, J=2.0 Hz, 1H), 6.70 (d, J=3.7 Hz, 1H), 6.01 (s, 1H), 5.47 (m, 1H), 4.79 (s, OH), 4.43 (s, OH), 3.90 (s, 3H), 3.78 (s, OH), 3.50 (s, OH), 3.16 (s, OH), 3.02-2.91 (m, 1H), 2.25 (m, 1H), 2.17 (m, 1H), 2.01 (s, 1H), 1.97-1.74 (m, 2H), 1.74-1.59 (m, 2H), 1.45 (m, 9H), 1.37-1.20 (m, 2H).

### Example 33: Compound 80

### Methyl 2-(2-(2-(4-((tert-butoxycarbonyl)amino)bicyclo[2.2.2]octan-1-yl)thiazole-4-carboxamido)acrylamido)acrylate

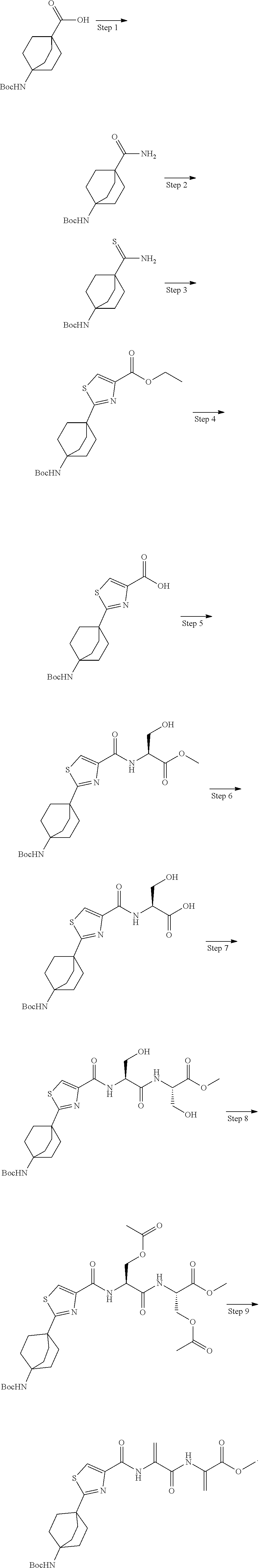

Step 1. tertbutyl (4-carbamoylbicyclo[2.222]octan-1-yl)carbamate was prepared following General experimental procedure 9. 4-((tert-butoxycarbonyl)amino)bicyclo[2.2.2]octane-1-carboxylic acid (1.0 g, 3.7 mmol) gave tert-butyl (4-carbamoylbicyclo[2.2.2]octan-1-yl)carbamate (1.2 g, 82% purity, 3.5 mmol, 95%)

Step 2. tert-butyl (4-carbamothioylbicyclo[2.2.2]octan-1-yl)carbamate was prepared following General experimental procedure 10. tert-butyl (4-carbamoylbicyclo[2.2.2]octan-1-yl)carbamate (1.2 g, 82 purity, 3.5 mmol) gave tert-butyl (4-carbamothioylbicyclo[2.2.2]octan-(-yl)carbamate (0.48 g, 1.7 mmol, 47)) as a white solid.

Step 3. Ethyl 2-(4-((tert-butoxycarbonyl)amino)bicyclo[2.2.2]octan-1-yl)thiazole-4-carboxylate was prepared following General experimental procedure 11. tert-butyl (4-carbamothioylbicyclo[2.2.2]octan-1-yl)carbamate (0.48 g, 1.7 mmol) gave ethyl 2-(4-((tert-butoxycarbonyl)amino)bicyclo[2.2.2]octan-1-yl)thiazole-4-carboxylate (0.31 g, 0.82 mmol, 490) as a white solid.¹H NMIR (400 l4z, CDCl₃) 6 8.00 (s, 1H), 4.37 (q, J=7.1 Hz, 2H), 2.12-1.99 (m, 6H), 1.99-1.91 (m, 6H), 1.41 (s, 9H), 1.36 (t, J=7.1 Hz, 3H).

Step 4. 2-(4-((tert-butoxycarbonyl)amino)bicyclo[2.2.2]octan-1-yl)thiazole-4-carboxylic acid was prepared following General experimental procedure 2. Ethyl 2-(4-((tert-butoxycarbonyl)amino)bicyclo[2.2.2]octan-1-yl)thiazole-4-carboxylate (0.31 g, 0.82 mmol) gave 2-(4-((tert-butoxycarbonyl)amino)bicyclo[2.2.2]octan-1-yl)thiazole-4-carboxylic acid (0.32 g, 90% purity, 0.82 mmol, quant.)

Step 5. methyl (2-(4-((tert-butoxycarbonyl)amino)bicyclo[2.2.2]octan-1-yl)thiazole-4-carbonyl)-L-serinate was prepared following General experimental procedure 4. 2-(4-((tert-butoxycarbonyl)amino)bicyclo[2.2.2]octan-1-yl)thiazole-4-carboxylic acid (0.32 g, 90% purity, 0.82 mmol) and methyl L-serinate hydrochloride (0.14 g, 1.1 eq., 0.90 mmol) gave methyl (2-(4-((tert-butoxycarbonyl)amino)bicyclo[2.2.2]octan-1-yl)thiazole-4-carbonyl)-L-serinate (0.33, 88% purity, 0.64 mmol, 78%)

Step 6. (2-(4-((tert-butoxycarbonyl)amino)bicyclo[2.2.2]octan-1-yl)thiazole-4-carbonyl)-L-serine was prepared following General experimental procedure 2. Methyl (2-(4-((tert-butoxycarbonyl)amino)bicyclo[2.2.2]octan-1-yl)thiazole-4-carbonyl)-L-serinate (0.33 g, 0.64 mmol) gave (2-(4-((tert-butoxycarbonyl)amino)bicyclo[2.2.2]octan-1-yl)thiazole-4-carbonyl)-L-serine (0.30 g, 0.66 mmol, quant).

Step 7. Methyl (2-(4-((tert-butoxycarbonyl)amino)bicyclo[2.2.2]octan-1-yl)thiazole-4-carbonyl)-L-seryl-D-serinate was prepared following General experimental procedure 4. (2-(4-((tert-butoxycarbonyl)amino)bicyclo[2.2.2]octan-1-yl)thiazole-4-carbonyl)-L-serine (0.30 g, 0.66 mmol) and methyl L-serinate hydrochloride (0.11 g, 1.1 eq., 0.73 mmol) gave methyl (2-(4-((tert-butoxycarbonyl)amino)bicyclo[2.2.2]octan-1-yl)thiazole-4-carbonyl)-L-seryl-D-serinate (0.30 g, 0.55 mmol, 83%).¹H NMR (400 MHz, CDCl₃) δ 8.25 (d, J=7.4 Hz, 1H), 7.96 (s, 1H), 7.67 (d, J=7.9 Hz, 1H), 4.69 (m, 2H), 4.36 (s, 1H), 4.17-4.08 (m, 1H), 4.08-3.92 (m, 2H), 3.88-3.81 (m, 1H), 3.79 (s, 3H), 3.77-3.70 (m, 1H), 3.61 (m, 1H), 2.07-1.92 (m, 12H), 1.42 (s, 9H).

Step 8. Methyl O-acetyl-N—(O-acetyl-N-(2-(4-((tert-butoxycarbonyl)amino)bicyclo[2.2.2]octan-1-yl)thiazole-4-carbonyl)-L-seryl)-L-serinate was prepared following General experimental procedure 5. Methyl (2-(4-((tert-butoxycarbonyl)amino)bicyclo[2.2.2]octan-1-yl)thiazole-4-carbonyl)-L-seryl-L-serinate (0.30 g, 0.55 mmol) and acetic anhydride (0.13 mL, 2.5 eq., 1.4 mmol) gave methyl O-acetyl-N-(0-acetyl-N-(2-(4-((tert-butoxycarbonyl)amino)bicyclo[2.2.2]octan-1-yl)thi azole-4-carbonyl)-L-seryl)-L-serinate (0.26 g, 0.41 mmol, 76%).

Step 9. Methyl 2-(2-(2-(4-((tert-butoxycarbonyl)amino)bicyclo[2.2.2]octan-1-yl)thiazole-4-carboxamido)acrylamido)acrylate was prepared following General experimental procedure 8. Methyl O-acetyl-N—(O-acetyl-N-(2-(4-((tert-butoxycarbonyl)amino)bicyclo[2.2.2]octan-1-yl)thiazole-4-carbonyl)-L-seryl)-L-serinate (0.15 g, 0.23 mmol) gave methyl 2-(2-(2-(4-((tert-butoxycarbonyl)amino)bicyclo[2.2.2]octan-1-yl)thiazole-4-carboxamido)acrylamido)acrylate (65 mg, 0.13 mmol, 55%) as a white solid. LCMS (22010199A TFA LCMS-5 Cl): RT: 1.783 min; Area: 99.9% (215 nm), 99.6% (306 nm); m z=505.2 [M+H]+

General procedure for solid phase peptide synthesis. Fmoc Rink amide AM resin (0.70 mmol/g) was swelled by washing with CH₂Cl₂ (2x 1 min, 10 mL/gram resin). The resin was washed with DMF (3x 1 min, 10 mL/gram resin) and treated with 20% piperidine in DMF (10 mL/gram resin) for 30 minutes. The resin was washed with DMF (3×1 min, 10 mL/gram resin). The resin was treated with a solution of Fmoc-AA(PG)—OH (3 eq.), HATU (3 eq.) and DiPEA (3 eq.) for 2-3 hours or 16 hours. The resin was washed with DMF (3×1 min, 10 mL/gram resin). The cycle of wash-deprotection-wash-coupling was repeated for everyone amino acid coupling. After completion of the coupling cycles the resin was treated with TFA:TIPS:water (95:2.5:2.5, 10 mL/gram resin) for 2 hours. The resin was removed by filtration and the filtrate was precipitated with MTBE:Heptane (1:1 v/v). After centrifugation (5 min, 3000 rpm), the supernatant was discarded and the residue was resuspended in MTBE:heptane (1:1 v/v) and centrifuged again (5 min, 3000 rpm). The supernatant was discarded and the pellet was taken up in water/MeCN 1:1. The solution was partially concentrated and lyophilized. The crude peptide was purified by automated reverse phase FCC.

### Example 34: Compound 81

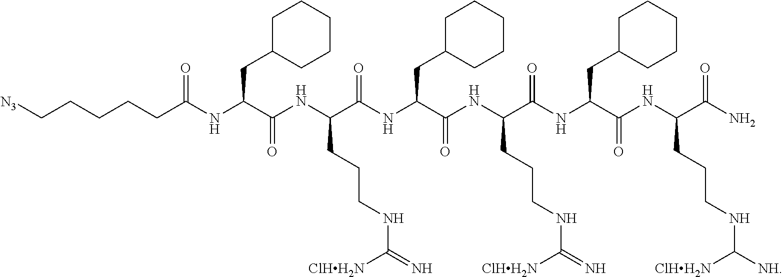

N₃(CH₂)₅C(O)-Cha-D-Arg-Cha-D-Arg-Cha-D-Arg-NH₂ was prepared following General procedure for solid phase peptide synthesis on 2.5 mmol scale. Purification using HCl buffers provided the desired HCl salt form. N₃(CH₂)sC(O)-Cha-D-Arg-Cha-D-Arg-Cha-D-Arg-NELEICI salt (0.58 g, 0.50 mmol, 20%) was isolated as a white solid. LCMS (30833 LCMS-6): RT: 2.403 min; Area: 78.0% (215 nm), 99.6% (ELSD); m z=1084.7 [M+H]+

### Example 35: Compound 82

### N₃(CH₂CH₂O)₆CH₂CH₂C(O)-Cha-D-Arg-Cha-D-Arg-Cha-D-Arg-NH₂ HCl salt

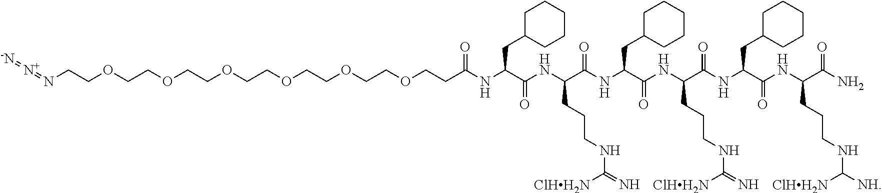

N₃(CH₂CH₂O)₆CH₂CH₂C(O)-Cha-D-Arg-Cha-D-Arg-Cha-D-Arg-NH₂ HCl salt was prepared following General procedure for solid phase peptide synthesis on 0.25 mmol scale. Purification using HCl buffers provided the desired HCl salt form.

N₃(CH₂CH₂O)₆CH₂CH₂C(O)-Cha-D-Arg-Cha-D-Arg-Cha-D-Arg-NH₂ HCl salt (39 mg, 28 μmol, 11%) was isolated as a white solid. LCMS (30833 LCMS-6): RT: 2.365 min; Area: 97.8% (215 nm), 99.6% (ELSD); m/z=1306.8 [M+H]⁺.

### Example 36: Compound 83

### N₃(CH₂CH₂O)₉CH₂CH₂C(O)-Cha-D-Arg-Cha-D-Arg-Cha-D-Arg-Mlz HCl salt

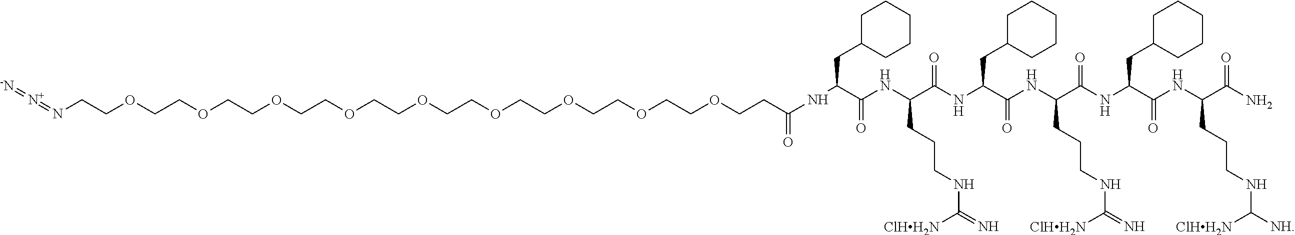

N₃(CH₂CH₂O)₉CH₂CH₂C(O)-Cha-D-Arg-Cha-D-Arg-Cha-D-Arg-NH₂ HCl salt was prepared following General procedure for solid phase peptide synthesis on 0.25 mmol scale. Purification using HCl buffers provided the desired HCl salt form.

N₃(CH₂CH₂O)₉CH₂CH₂C(O)-Cha-D-Arg-Cha-D-Arg-Cha-D-Arg-NH₂ HCl salt (34 mg, 22 μmol, 8.8%) was isolated as a white solid. LCMS (30833 LCMS-6): RT: 2.415 min; Area: 98.4% (215 nm), 99.8% (ELSD); m z=1438.9 [M+H]+

General procedure for conjugation chemistry between azido-peptide and Compound 49. To a solution of Compound 49 in DMF/water (1: 1, 2 mL) was added the azido-peptide and the resulting mixture was stirred for 3 hours. The mixture was directly purified using automated reverse phase FCC.

### Example 37: Compound 84

Compound 49 coupled to Compound 82

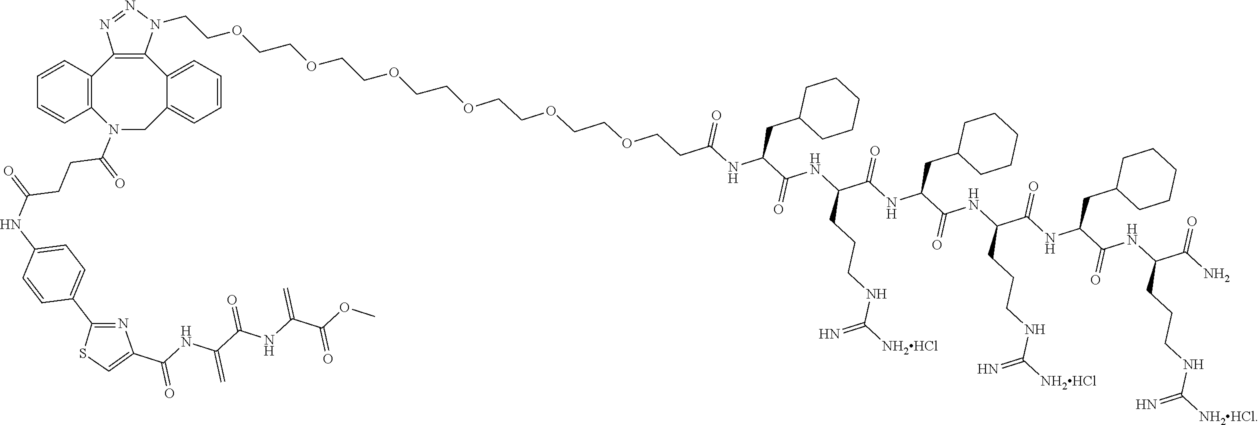

Compound 84 was prepared following General procedure for conjugation chemistry between azido-peptide and 49. Compound 49 (3.0 mg, 4.5 μmol) and Compound 82 (6.4 mg, 4.5 μmol) gave Compound 84 (4.3 mg, 2.1 μmol, 46%) as a white solid. LCMS (30833 LCMS-6): RT: 2.791 min; Area: 81.9% (215 nm), 98.1% (ELSD); m/z=1966.2 [M+H]+

### Example 38: Compound 85

Compound 49 coupled to Compound 83

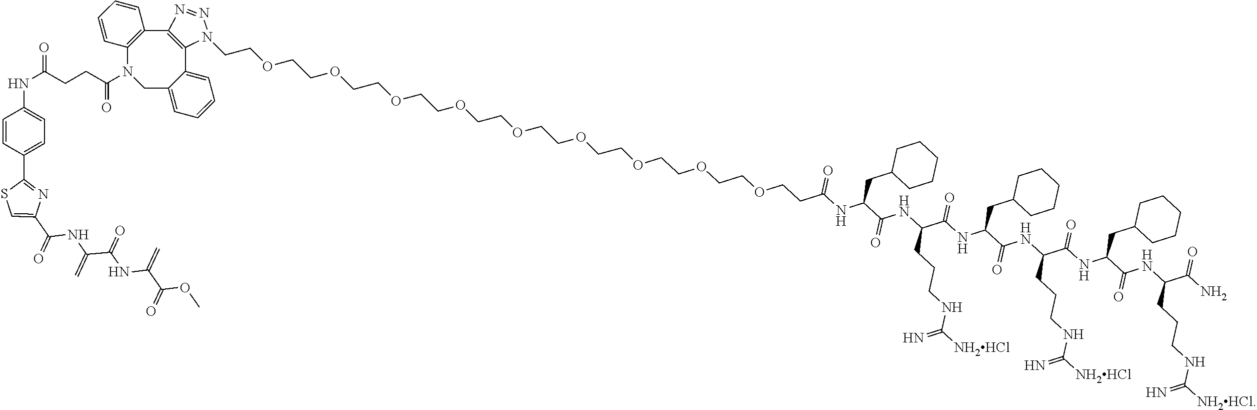

Compound 85 was prepared following General procedure for conjugation chemistry between azido-peptide and.Compound 49 (3.0 mg, 4.5 μmol) and Compound 83 (7.0 mg, 4.5 μmol) gave Compound 85 (4.3 mg, 2.0 μmol, 43%) as a white solid. LCMS (30833 LCMS-6): RT: 2.802 min; Area: 59.7% (215 nm), 85.5% (ELSD); m/z=1049.7 [M+2H]2.

### Example 39: Prophetic Assays

To test the mechanism of binding and to explore the binding target, cell treatments and western blotting will be carried out as in Cunniff et al. 2015. Covalent crosslinking of enzymes is detectable by protein western blotting using specific antibodies.

Briefly, human tumor cell lines (HMESO cell line derived from a patient with malignant mesothelioma) will be cultured in appropriate medium and treated with varying concentrations of test compounds for 24 hours (0.1 μM-100 μM). After 24 hours of exposure to test compounds, cellular lysates will be generated in standard lysis buffer (RIPA Buffer). Protein abundance will be quantified, and equal protein concentrations will be separated by SDS-Polyacrylamide Gel Electrophoresis (SDS-PAGE). Protein western blotting will be conducted using antibodies specific to proteins, such as PRX1, PRX2, PRX3 and PRX4. Covalent crosslinking modifications are detectable by the presence of an ˜46 kD antibody reactive species on the protein western blot.

Cell death assays will be conducted as in Nelson et al. 2021. Briefly, human tumor cell lines (HMESO cell line derived from a patient with malignant mesothelioma) will be cultured in 96-well plates and incubated with test compounds for 48 hours. The amount of residual cell material will be stained with crystal violet and total cell counts will conducted to determine % cell viability.

### Example 40: Cell Viability Study

Cell Lines were plated in 96-well plates (Corning, Kennebunk, ME, USA) at a density of 2500 cells per well. The following day, cells were treated with test compounds diluted in complete media followed by incubation for 48 h. Post-incubation cells were washed with PBS (Corning Cellgro, Manassas, VA, USA), fixed with 3.0% formaldehyde (Fisher BioReagents, Fair Lawn, NJ, USA) in PBS, and stained for 30 min with 0.1% crystal violet (Acros Organics, Fair Lawn, NJ, USA) in water. Crystal violet stain was removed, and plates were washed with FEO and allowed to dry. To quantify cell viability, plates were imaged using the Lionheart Plate reader (BioTek Instruments, Winooski, VT, USA) and/or analyzed by absorbance at 540 nm (crystal violet dye dissolved in 100% methanol) using the Synergy HTX plate reader (BioTek Instruments, Winooski, VT, USA). To determine the effective cytotoxic concentration (ICso) of test compounds, the data were plotted using a 4-parameter non-linear regression model using GraphPad Prism7 software (GraphPad Software, San Diego, CA, USA).

Cell viability assay data in HMESO mesothelioma cells treated with compounds (1)—HCl or (5) HCl are summarized in the below Table 2. ICso (Concentration of drug required to kill 50% f cells) is shown in pM concentration

TABLE 2

| | TS | (1)-HC1 | (5)-HC1 | DMSO |
|---|---|---|---|---|
| IC₅₀ | 1.013 | 747.8 | 1.930* | Unstable |

*95% confidence interval (CI) of 1.699-2.187

### Example 41: Crosslinking Study

Malignant Mesothelioma (MM) cells (H-MESO cell line) were plated into 6-well plates in complete tissue culture media. Cells were allowed to adhere for 24 hours before being treated with indicated concentrations of thiostrepton (TS), (1) or (5) (DMSO Stocks) for 24 hours. Cell lysates were generated using standard RIPA buffer, protein concentrations were determined using a Bradford Assay and 20 μg of total protein per sample were separated by reducing SDS-Polyacrylamide Gel Electrophoresis. Proteins were transferred to a PVDF membrane, blocked with 5% Bovine Serum Albumin (BSA) for 1 hour and incubated with PRX3 primary antibody overnight at 4° C. in IX Tris Buffered Saline with Tween (TBST). Membranes were washed 3X with IX TBST and incubated with horseradish peroxidase conjugated (HRP) secondary antibody for 1 hour at room temperature. Membranes were washed 3x in IX TBST and HRP signal was developed using Enhanced Chemiluminescence and visualized on a GE digital imager.

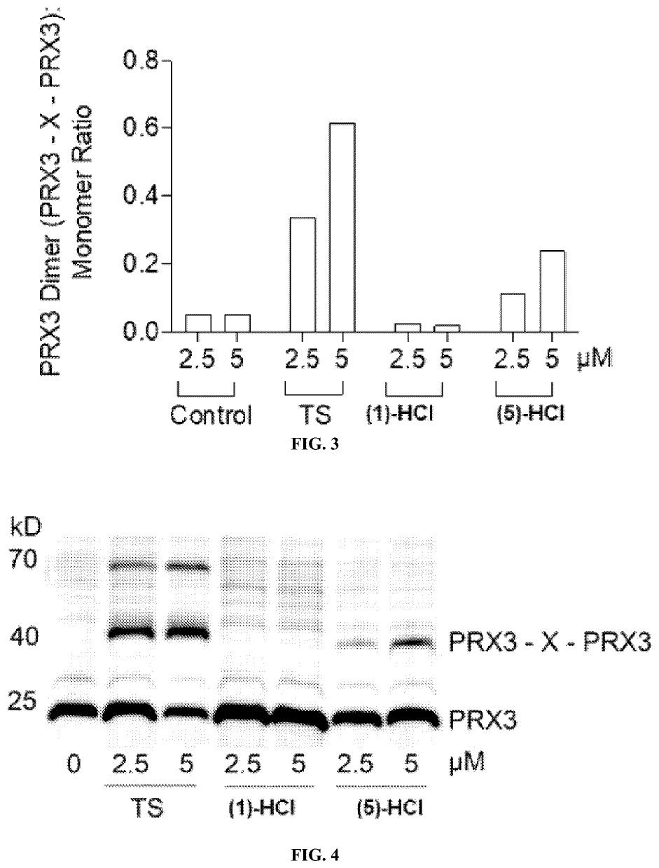

Treatment of cells with 2.5 or 5 μM of TS resulted in covalent PRX3 crosslinking (PRX3 —X —PRX3). Similar, yet less robust, results were observed for (5)-treated cells (FIG. 3). No PRX3 —X —PRX3 crosslinking was observed in cells treated with (1) (also FIG. 3).

### Example 42: Cytotoxic Activity of Test Compounds in Malignant Mesothelioma Cell Lines

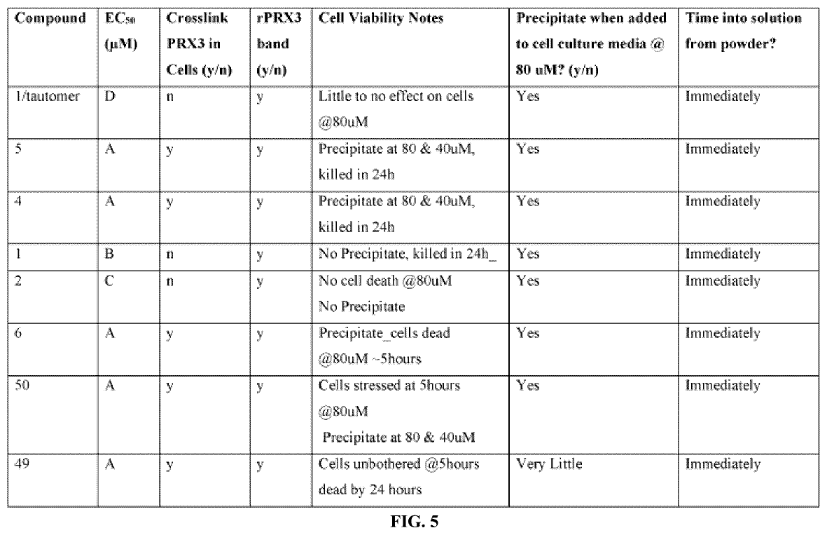

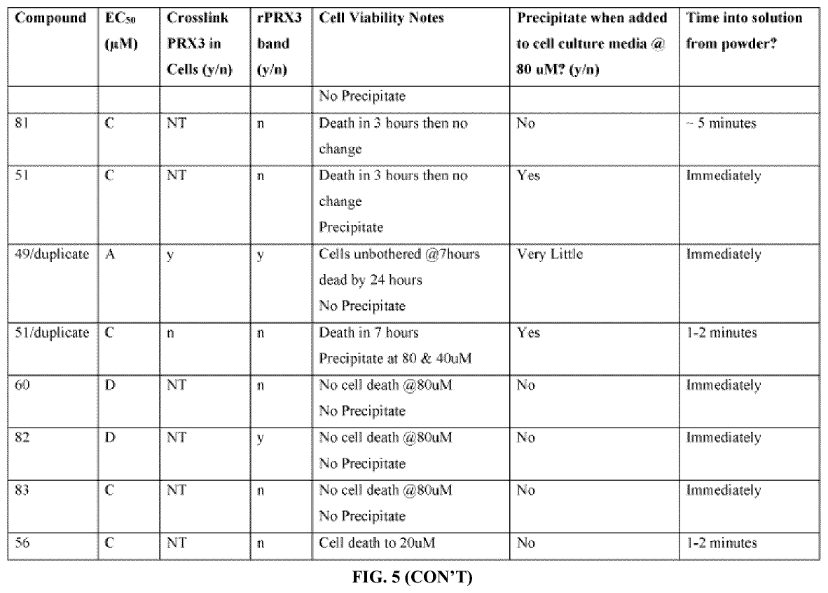

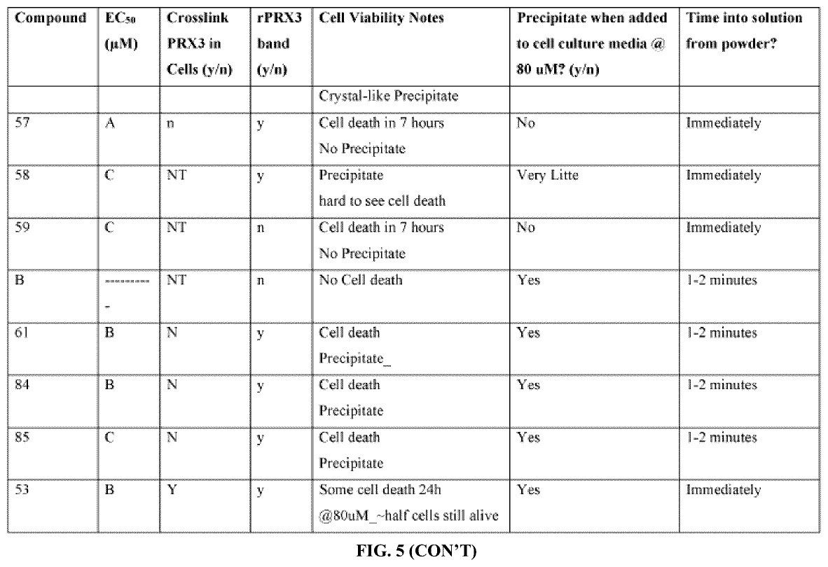

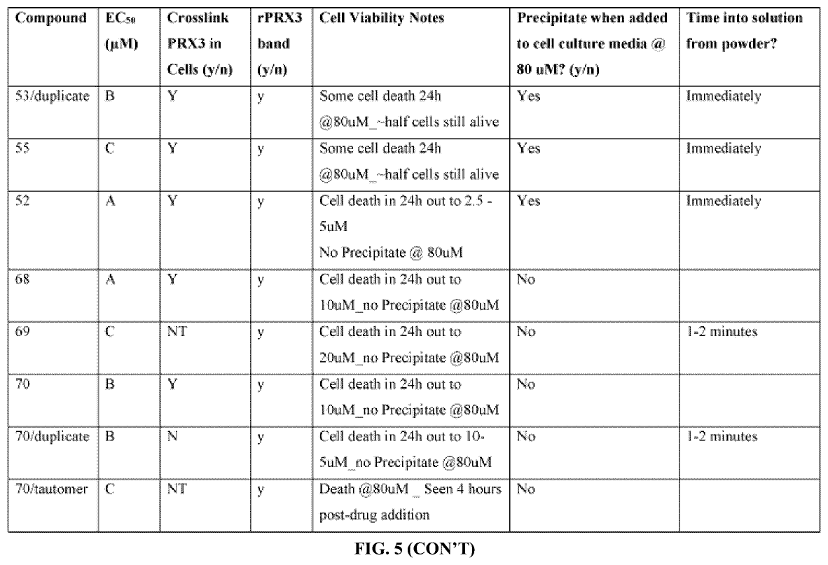

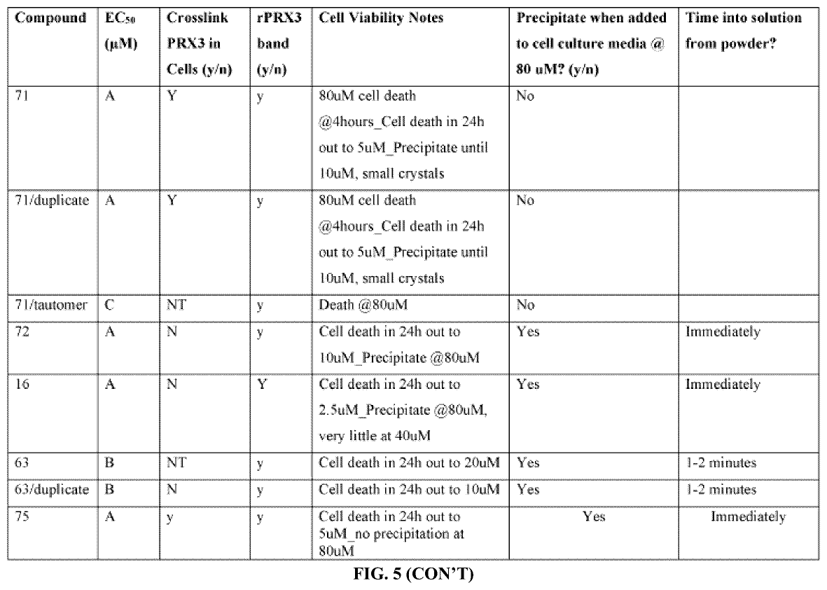

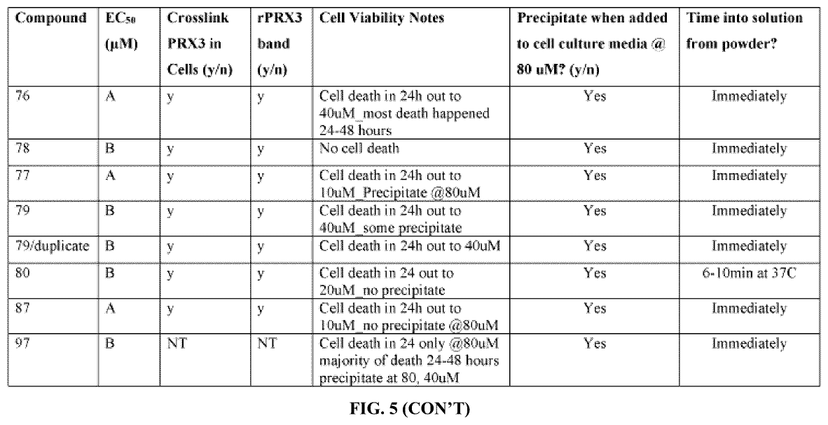

Malignant mesothelioma (HMESO cell line) cells were plated in 96-well plates (Corning, Kennebunk, ME, USA) at a density of 2500 cells per well. The following day, cells were treated with test compounds diluted in complete media followed by incubation for 48 h (in technical duplicates). Post-incubation cells were washed with PBS (Corning Cellgro, Manassas, VA, USA), fixed with 3.0% formaldehyde (Fisher BioReagents, Fair Lawn, NJ, USA) in PBS, and stained for 30 min with 0.1% crystal violet (Acros Organics, Fair Lawn, NJ, USA) in water. Crystal violet stain was removed, and plates were washed with H2O and allowed to dry. To quantify cell viability, plates were imaged using the Lionheart Plate reader (BioTek Instruments, Winooski, VT, USA) and/or analyzed by absorbance at 540 nm (crystal violet dye dissolved in 100% methanol) using the Synergy HTX plate reader (BioTek Instruments, Winooski, VT, USA). To determine the effective cytotoxic concentration (EC₅₀) of test compounds the data were plotted using a 4-parameter non-linear regression model using GraphPad Prism7 software (GraphPad Software, San Diego, CA, USA). Results are tabulated in FIG. 5.

### Example 43: Covalent Crosslinking of Recombinant Peroxiredoxin 3 (rPRX3) by Test Compounds

Master Mix reagents in Table 3 were combined for a IX reaction in an Eppendorf tube on ice. The reaction was scaled by the number of test compounds being tested. 16 μL of master mix were added to a new Eppendorf tube containing 1 μL of test compound (10 mM stock diluted in DMSO) and mixed by gentle flicking and quick centrifugation at 1,000 RPM. Reactions were incubated at 37° C. for 18 hours. Reactions were removed from incubation and quenched by addition of 2 μL of Laemmli buffer containing 0.2 M dithiothreitol (DTT) and 10% sodium dodecyl sulfate. Samples were boiled at 98° C. for 5 minutes. Samples were separated by polyacrylamide gel electrophoresis, transferred to a PVDF membrane, and subjected to protein western blotting using an anti-PRX3 antibody (AbFrontier, LF-PA0255). Membranes were incubated with ECL Reagent (ThermoScientific, Rockford, TL, USA) and visualized using a GE Amersham Imager chemiluminescent detection system. Unmodified rPRX3 was visualized as a single band at −23 kDa and rPRX3 covalently modified by test compounds runs as a −45 kDa band. This is a qualitative assay evaluating the presence or absence of the −45 kDA band. Qualitative results are tabulated in FIG. 5.

TABLE 3
Master Mix

| Reagent | uL |
|---|---|
| 20 nM Tris pH 8 | 11 |
| rPRX (1 mg/ml) | 2 |
| 0.5M TCEP | 3 |

### Example 44: Covalent Crosslinking of Peroxiredoxin 3 (PRX3) in Malignant Mesothelioma Cells by Test Compounds

Human malignant mesothelioma (HMESO cell line) cells were plated in 6 well plates at a density of 200,000 cells per well. After 24 hours, cells were treated with test compounds diluted in DMSO and cell culture media. Cell lysates were harvested at 24 hours post treatment using RIPA buffer (50 mM Tris-HCl, 150 mM NaCl, 1 mM EDTA, 1% NP-40, 0.25% Sodium deoxycholate, 0.1% sodium dodecyl sulfate, in deionized (DI) water) for reducing samples to be analyzed by reducing SDS-PAGE. Protein concentrations were determined via Bradford Assay (ThermoScientific, Rockford, IL, USA). Lysates (15 pg protein/well) were resolved by SDS-PAGE under reducing conditions on 4-12% gradient Bis-Tris Midi gel (Invitrogen, Carlsbad, CA, USA) at constant 200 V for 50 m. The gel was transferred to a PVDF membrane at constant 1 A for 50 min, blocked with 5% BSA diluted in 1*Tris-buffered saline with 1% Tween-20 (TBS-T) for a minimum of 1 hour, and incubated with anti-PRX3 antibody in 5% BSA TBS-T at 4° C. overnight. The membrane was washed with 1*TBS-T for 1 hour, incubated with appropriate secondary antibody 1 hour, and washed again with 1*TBS-T for 1 hour. Membranes were incubated with ECL Reagent (ThermoScientific, Rockford, IL, USA) and visualized using a GE Amersham Imager chemiluminescent detection system. Qualitative results are tabulated in FIG. 5.

### Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

### Equivalents

Furthermore, the invention encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permit the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

### Illustrations

As used below, any reference to a series of illustrations is to be understood as a reference to each of those examples disjunctively (e.g., “Illustrations 1-4” is to be understood as “Illustrations 1, 2, 3, or 4”).

Illustration 1 is a compound of Formula I

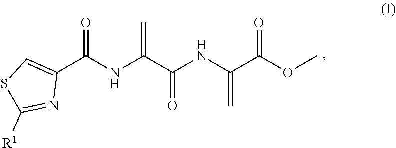

wherein R¹ is an aryl, heteroaryl, cycloalkyl or heterocycle, which aryl, heteroaryl, cycloalkyl or heterocycle is optionally substituted with one or more selected from alkyl, carboxy, carbamate, urea, amide, amino, ether, ester and halo; and
wherein when R¹ is pyridine or pyrazine, said pyridine or pyrazine is substituted with one or more selected from alkyl, carboxy, carbamate, urea, amide, amino, ether, ester and halo,
or a pharmaceutically acceptable salt thereof.

Illustration 2 is the compound of illustration 1, wherein said aryl, heteroaryl, cycloalkyl or heterocycle, is substituted with one or more selected from alkyl, carboxy, carbamate, urea, amide, amino, ether, ester and halo.

Illustration 3 is the compound of illustration 1, wherein said aryl, heteroaryl, cycloalkyl or heterocycle, is substituted with carbamate or amide.

Illustration 4 is the compound of illustration 1, wherein said aryl, heteroaryl, cycloalkyl or heterocycle, is substituted with an alkylcarbamate.

Illustration 5 is the compound of illustration 1, wherein R¹ is a group having a structure of

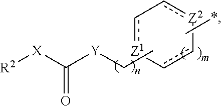

wherein:
n is 0, 1, 2 or 3;
m is 0, 1 or 2;
X is absent or is O, NR³, or CH₂;
Y is absent or is O, NR³, or CH₂;
Z¹ and Z² are each independently O, N, or C;
R² is alkyl (e.g., having from 1 to 8 carbon atoms, linear or branched), wherein said alkyl is optionally substituted (e.g., with halo, amino, ether, alkoxy, or carbamate), or heterocycle; and
R³ is H or alkyl (e.g., having from 1 to 8 carbon atoms, linear or branched),
wherein * denotes the connection of the group in the compound of Formula I,
or a pharmaceutically acceptable salt thereof.

Illustration 6 is the compound of illustration 5, wherein Z¹ and Z² are each independently N or C.

Illustration 7 is the compound of illustration 1 or illustration 5, wherein the compound is selected from the group consisting of

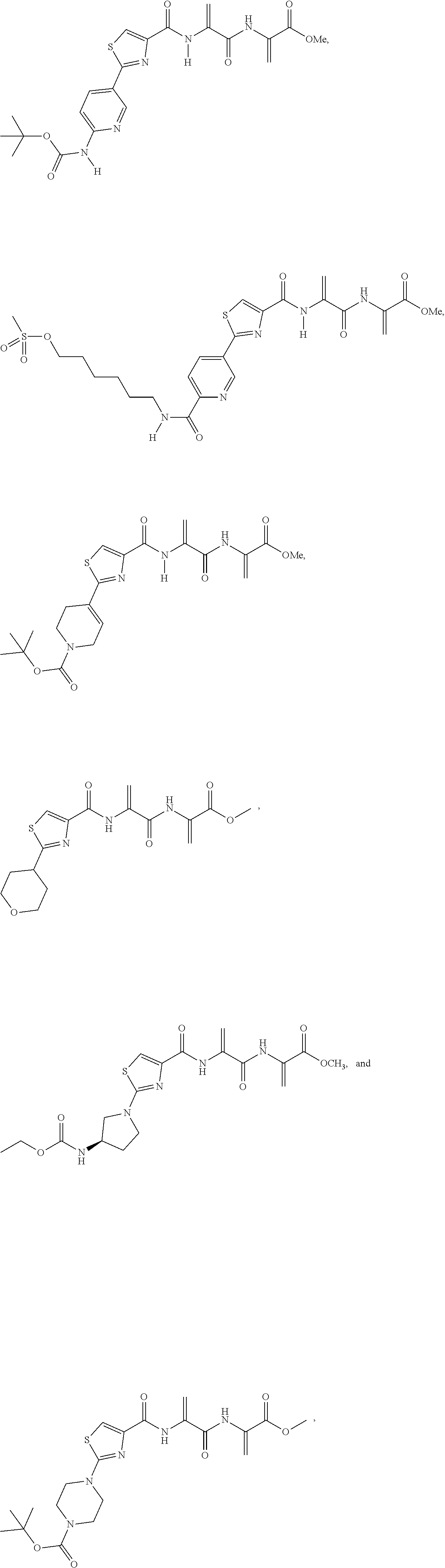

or a pharmaceutically acceptable salt thereof.

Illustration 8 is the compound of illustration 1 or illustration 5, wherein the compound is selected from the group consisting of

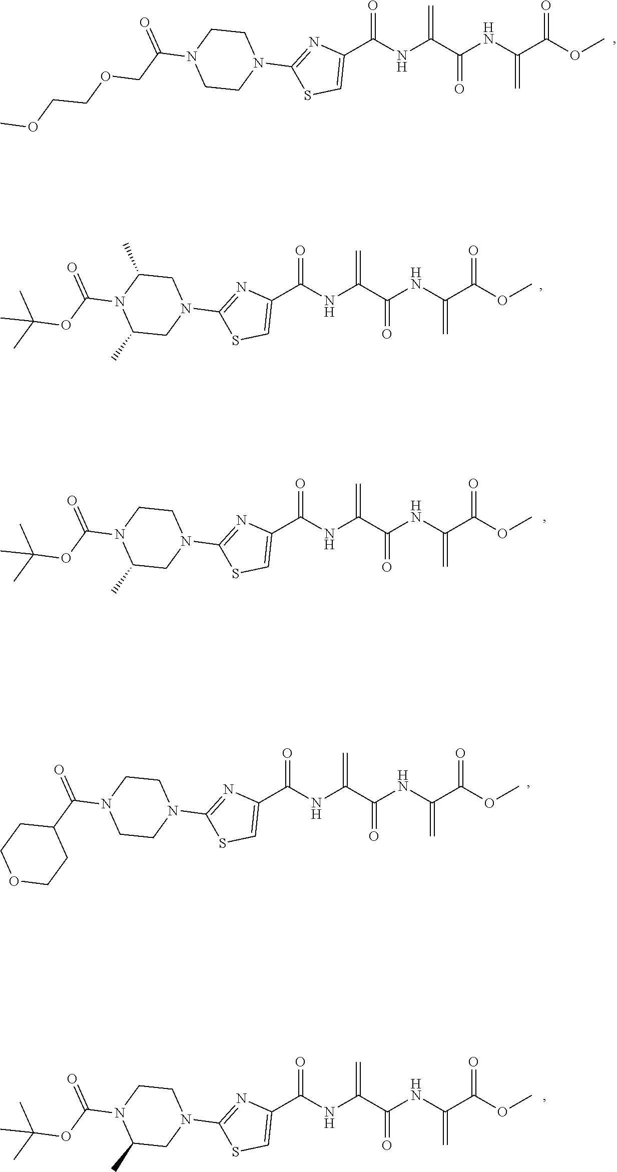

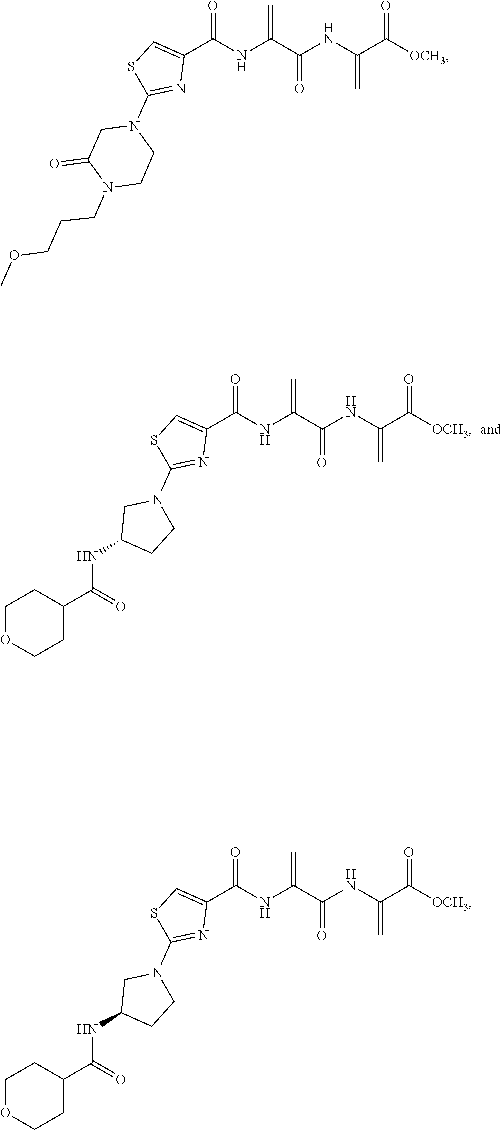 [SMILES: C=C(NC(=O)c1csc(N2CCN(CCCOC)C(=O)C2)n1)C(=O)NC(=C)C(=O)OC ; C=C(NC(=O)c1csc(N2CC[C@H](NC(=O)C3CCOCC3)C2)n1)C(=O)NC(=C)C(=O)OC ; C=C(NC(=O)c1csc(N2CC[C@@H](NC(=O)C3CCOCC3)C2)n1)C(=O)NC(=C)C(=O)OC]

or a pharmaceutically acceptable salt thereof.

Illustration 9 is the compound of illustration 1 or illustration 5, wherein R¹ is a group having a structure of

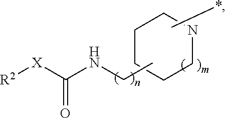

wherein:
n is 0, 1, 2 or 3;
m is 0, 1 or 2;
X is O or CH₂; and
R² is alkyl (e.g., having from 1 to 8 carbon atoms, linear or branched), wherein said alkyl is optionally substituted (e.g., with halo, amino, ether, alkoxy, or carbamate), or heterocycle; and
wherein * denotes the connection of the group in the compound of Formula I,
or a pharmaceutically acceptable salt thereof.

Illustration 10 is the compound of illustration 1 or illustration 5, wherein R¹ is a group having a structure of

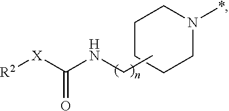

wherein:
n is 0, 1, 2 or 3;
X is O or CH₂; and
R² is alkyl (e.g., having from 1 to 8 carbon atoms, linear or branched), wherein said alkyl is optionally substituted (e.g., with halo, amino, ether, alkoxy, or carbamate), or heterocycle; and
wherein * denotes the connection of the group in the compound of Formula I,
or a pharmaceutically acceptable salt thereof.

Illustration 11 is the compound of illustration 1, 5 or 9, wherein the compound is selected from the group consisting of

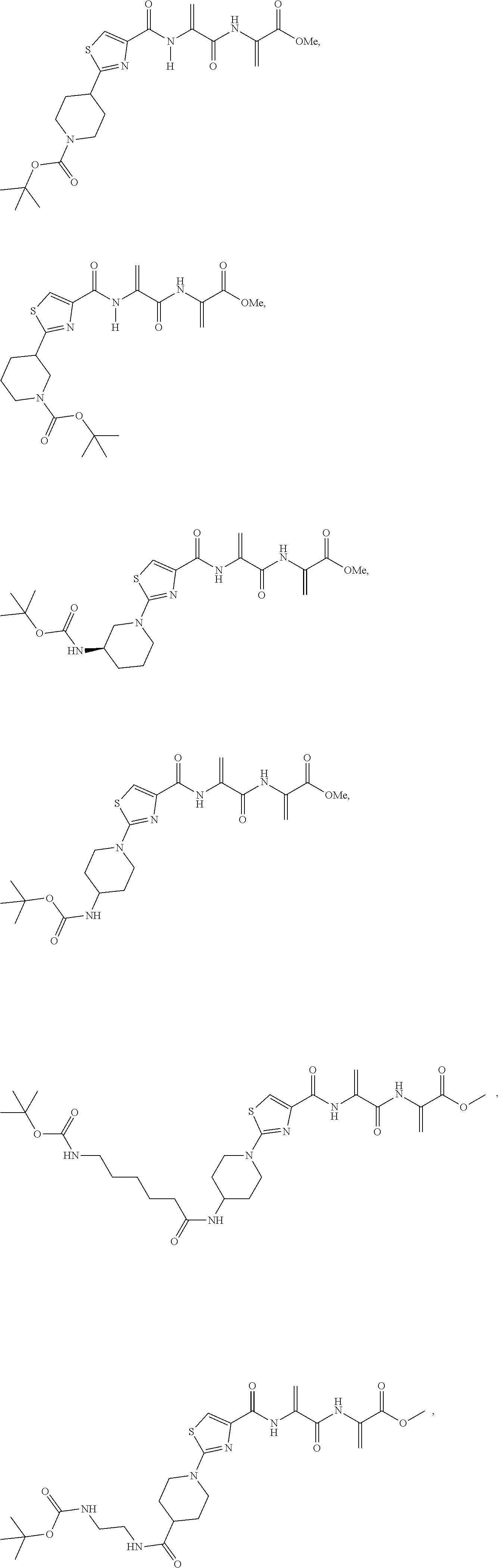

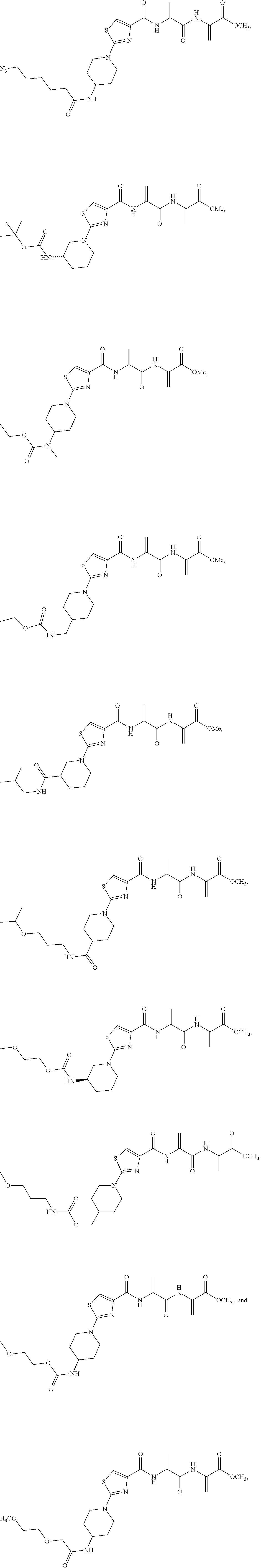

or a pharmaceutically acceptable salt thereof.

Illustration 12 is the compound of illustration 1, 5, 9 or 10, wherein the compound is selected from the group consisting of

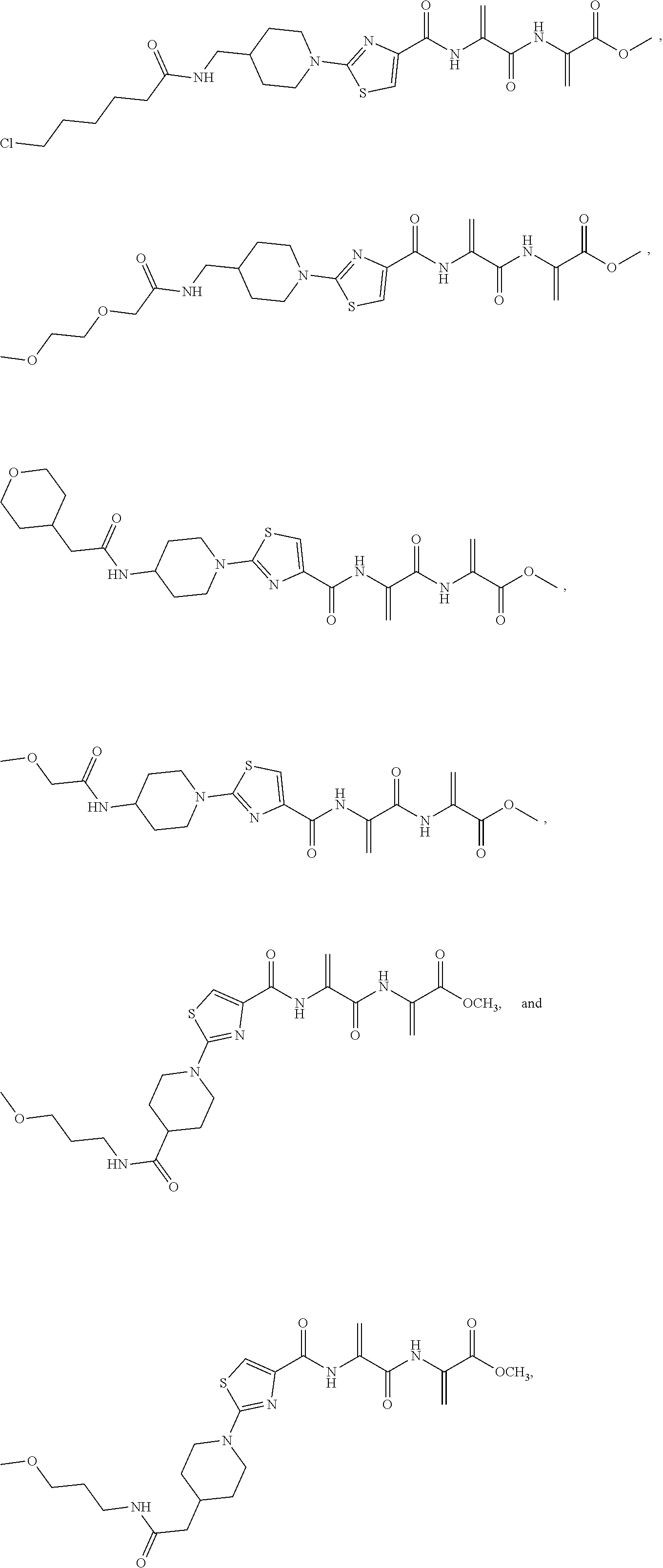

or a pharmaceutically acceptable salt thereof.

Illustration 13 is the compound of illustration 1, 5, 9 or 10, wherein the compound is selected from the group consisting of

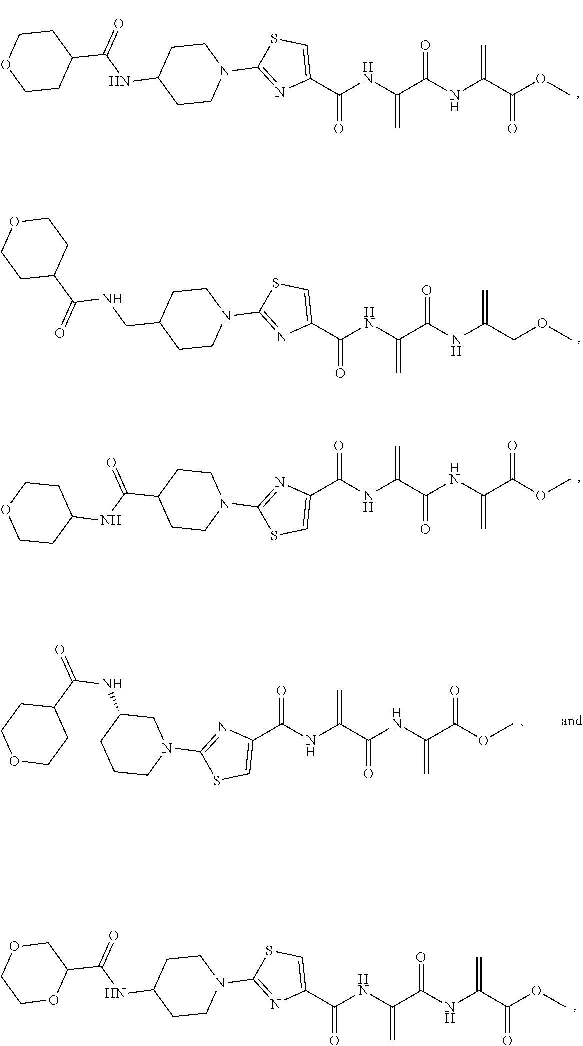

or a pharmaceutically acceptable salt thereof.

Illustration 14 is the compound of illustration 1 or 5, wherein R¹ is a group having a structure of

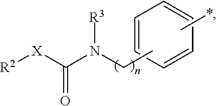

wherein:
n is 0, 1, 2 or 3;
X is O or CH₂;
R² is alkyl (e.g., having from 1 to 8 carbon atoms, linear or branched), wherein said alkyl is optionally substituted (e.g., with halo, amino, ether, alkoxy, or carbamate), or heterocycle; and
R³ is H or alkyl (e.g., having from 1 to 8 carbon atoms, linear or branched),
wherein * denotes the connection of the group in the compound of Formula I,
or a pharmaceutically acceptable salt thereof.

Illustration 15 is the compound of illustration 1, 5 or 14, wherein the compound is selected from the group consisting of

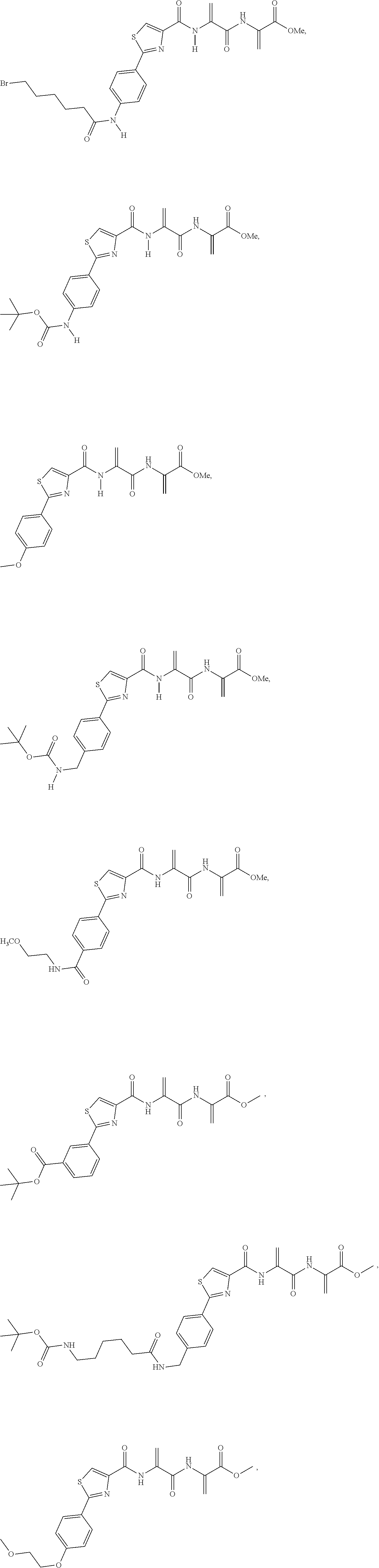

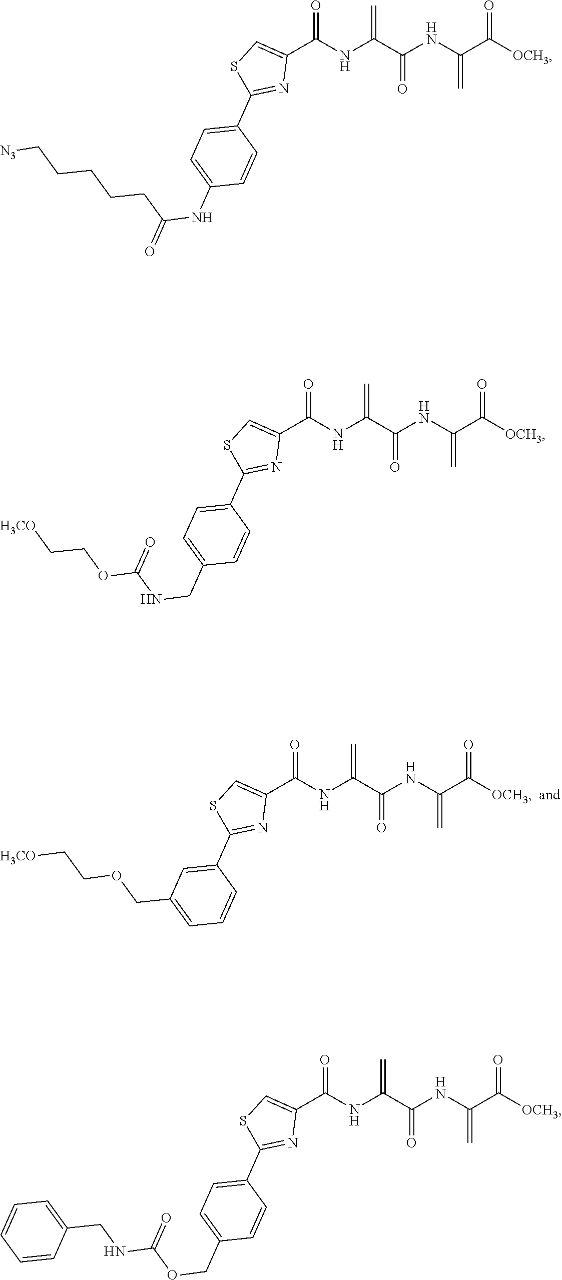

or a pharmaceutically acceptable salt thereof.

Illustration 16 is the compound of illustration 1, 5 or 14, wherein the compound is selected from the group consisting of

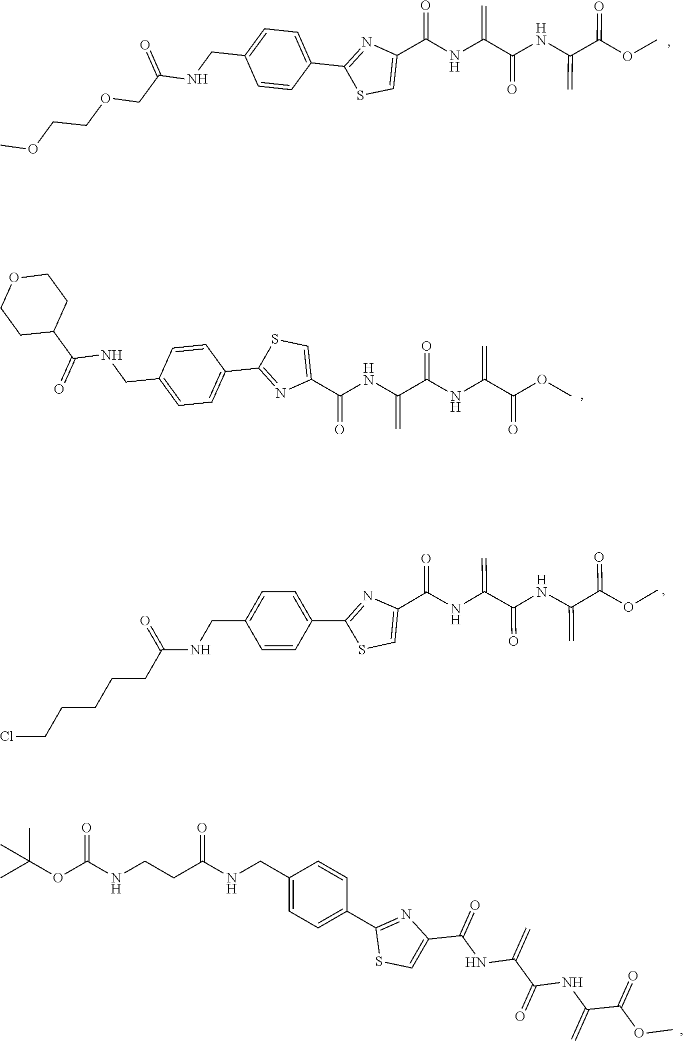

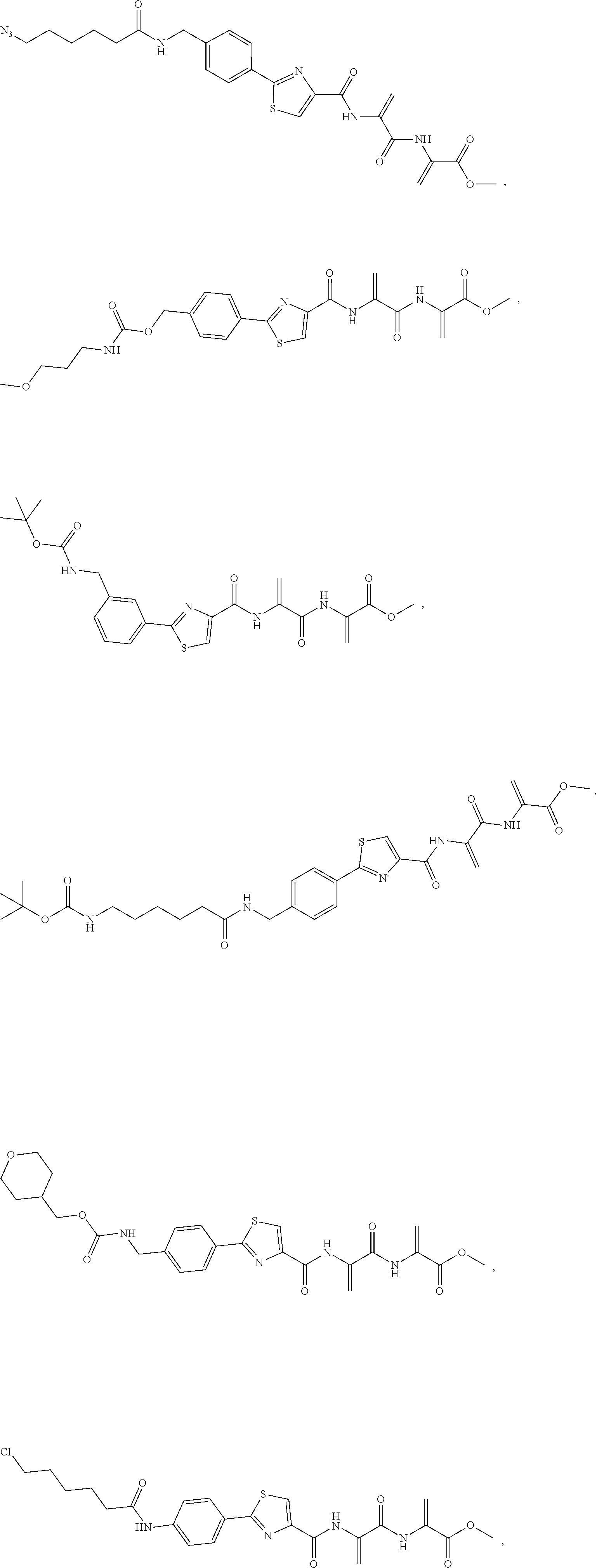

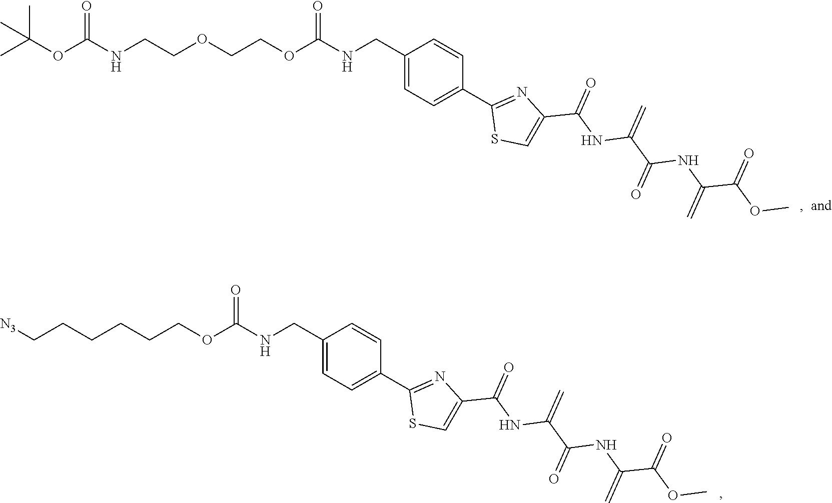 [SMILES: C=C(NC(=O)c1csc(-c2ccc(CNC(=O)OCCOCCNC(=O)OC(C)(C)C)cc2)n1)C(=O)NC(=C)C(=O)OC ; C=C(NC(=O)c1csc(-c2ccc(CNC(=O)OCCCCCCN=[N+]=[N-])cc2)n1)C(=O)NC(=C)C(=O)OC]

or a pharmaceutically acceptable salt thereof.

Illustration 17 is the compound of illustration 1 or 5, wherein the compound is selected from the group consisting of

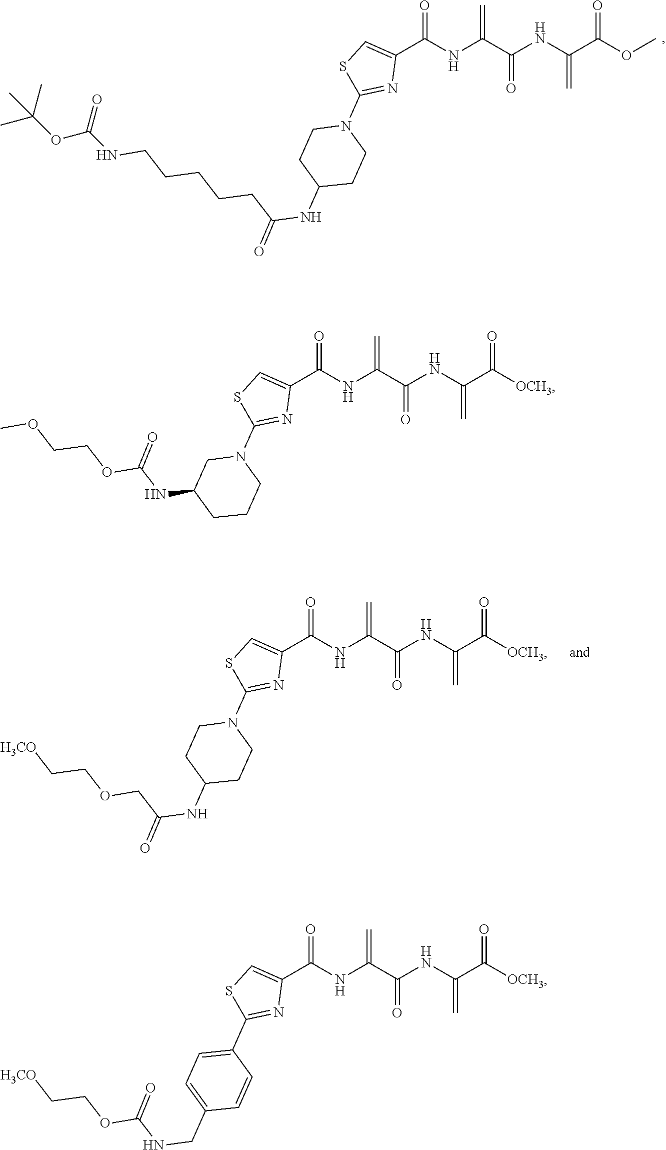

or a pharmaceutically acceptable salt thereof.

Illustration 18 is a pharmaceutical composition comprising a compound or pharmaceutically acceptable salt of any one of illustration 1-17.

Illustration 19 is the composition of illustration 18, wherein said composition is formulated for oral or parenteral (e.g. intravenous, intrapleural, intraperitoneal or intraovarian) administration.

Illustration 20 is the composition of illustration 18, wherein said composition is formulated for oral administration and is in the form of a capsule, cachet, lozenge, or tablet.

Illustration 21 is the composition of any one of illustration 18-20, wherein said formulation is provided in unit dosage form of from 1 mg to 10 grams of the compound, pharmaceutically acceptable salt or prodrug.

Illustration 22 is a method treating cancer in a subject in need thereof, comprising administering to the subject a therapeutically effective amount of a compound or pharmaceutically acceptable salt of any one of illustration 1-17.

Illustration 23 is the method of illustration 22, wherein the cancer has PRX3 expression.

Illustration 24 is the method of illustration 22 or illustration 23, wherein said subject is a human subject.

Illustration 25 is the method of illustration 22 or illustration 23, wherein said subject is a non-human animal subject (e.g. non-human mammalian subject).

Illustration 26 is the method of any one of illustration 22-25, wherein said administering is carried out by administering a pharmaceutical composition comprising said compound or pharmaceutically acceptable salt.

Illustration 27 is the method of any one of illustration 22-26, wherein said administering further comprises administering bortezomib, carboplatin, paclitaxel, an immunotherapy agent, or a combination thereof.

Illustration 28 is the method of any one of illustration 22-27, wherein said administering further comprises administering doxorubicin.

Illustration 29 is a method of inhibiting PRX3 in a subject in need thereof, comprising administering to said subject a therapeutically effective amount of a compound or pharmaceutically acceptable salt of any one of illustration 1-17.

The foregoing is illustrative of the present invention, and is not to be construed as limiting thereof. The invention is defined by the following claims, with equivalents of the claims to be included therein.

What is claimed is:

1. A compound having the structure of Formula (IA) 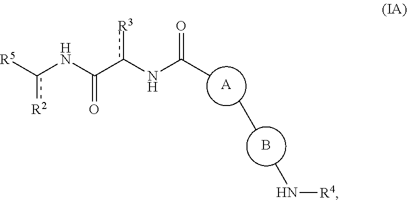
or a pharmaceutically acceptable salt thereof, wherein:
R¹ is —NH₂, —NH(CH₃), —O—CH₃, or —NH—CH₂—C(O)—NH₂;
R² is —H, —CH₃, ═CH₂, or ═CH(alkyl);
R³ is —H, —CH₃, ═CH₂, or ═CH(alkyl);
R⁵ is —C(O)—R¹ or —CN;
Ring A is aryl, heteroaryl, cycloalkyl, or heterocyclyl;
Ring B is absent or present and, when present, is aryl, heteroaryl, cycloalkyl, or heterocyclyl;
R⁴ is hydrogen, a protecting group, —C(O)—CH₃, —L′, or —L—Y;
L′, when present, is a reactive linker moiety;
L, when present, is a linker moiety;
Y, when present, is a mitochondrial targeting moiety; each [symbol] is independently a single bond or a double bond; and any hydrogen atom is optionally replaced with a deuterium.

2. The compound of claim 1 wherein R⁵ is —C(O)—R¹; and R¹ is —OCH₃.

3. The compound of claim 1, wherein R⁵ is —C(O)—R¹; and R¹ is —NH₂.

4. The compound of claim 1, wherein, when Ring A is polycyclic, then Ring B is absent.

5. The compound of claim 1 having the structure of Formula (IA-1): 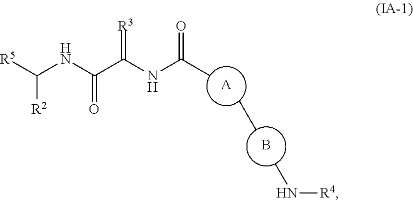
wherein
R² is —H or —CH₃; and R³ is ═CH₂ or ═CH(alkyl).

6. The compound of claim 5, wherein R² is —H and R³ is ═CH₂, or R² is —CH₃ and R³ is ═CH₂.

7. The compound of claim 1 having the structure of Formula (IA-2) 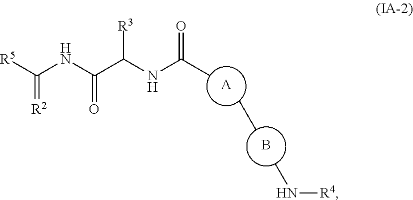
wherein
R² is ═CH₂ or ═CH(alkyl); and
R³ is —H or —CH₃.

8. The compound of claim 7, wherein R² is ═CH₂ and R³ is —H, or R² is ═CH₂ and R³ is —CH₃.

9. The compound of claim 1 having the structure of Formula (IA-3) 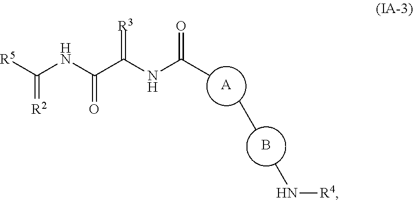
wherein
R² is ═CH₂ or ═CH(alkyl); and
R³ is ═CH₂ or ═CH(alkyl).

10. The compound of claim 9, wherein R² is ═CH₂ and R³ is ═CH₂, R² is ═CH₂ and R³ is ═CH(CH₃), or R² is ═CH(CH₃) and R³ is ═CH₂.

11. The compound of claim 1 having the structure of Formula (IB) 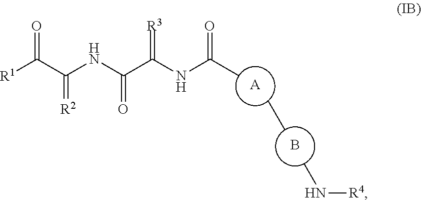
or a pharmaceutically acceptable salt thereof, wherein:
R¹ is —NH₂ or —O—CH₃;
R² is CH₂ or CH(alkyl);
R³ is CH₂ or CH(alkyl);
Ring A is heteroaryl, cycloalkyl, or heterocyclyl;
Ring B is aryl, heteroaryl, cycloalkyl, or heterocyclyl;
R⁴ is hydrogen, a protecting group, —C(O)—CH₃, —L′, or —L—Y;
L′, when present, is a reactive linker moiety;
L, when present, is a linker moiety;
Y, when present, is a mitochondrial targeting moiety; and any hydrogen atom is optionally replaced with a deuterium.

12. The compound of claim 11, wherein R² and R³ are different.

13. The compound of claim 11, wherein R² and R³ are the same.

14. The compound of claim 11, wherein R² is CH(Me) or —CH₂.

15. The compound of claim 11, wherein R³ is CH(Me) or —CH₂.

16. The compound of claim 13, wherein R² and R³ are each CH_{2.}

17. The compound of claim 1, wherein Ring A is a 5-membered ring or a 5-membered ring fused to a second ring.

18. The compound of claim 1, wherein Ring A is a 5-membered heteroaryl.

19. The compound of claim 18, wherein Ring A is thiazolyl, thiophenyl, oxazolyl, or imidazolyl.

20. The compound of claim 19, wherein Ring A is thiazolyl, thiophenyl, or oxazolyl.

21. The compound of claim 20, wherein Ring A is thiazolyl.

22. The compound of claim 18, wherein Ring A is 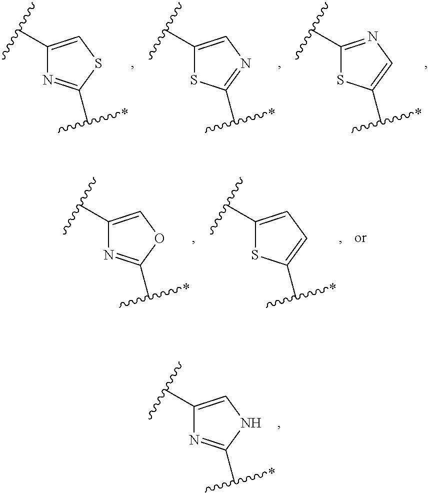 [SMILES: *c1csc(*)n1 ; *c1cnc(*)s1 ; *c1cnc(*)s1 ; *c1coc(*)n1 ; *c1ccc(*)s1 ; *c1c[nH]c(*)n1]
wherein * denotes a bond to Ring B.

23. The compound of claim 18, wherein Ring A is 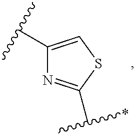 [SMILES: *c1csc(*)n1]
wherein * denotes a bond to Ring B.

24. The compound of claim 1, wherein Ring A is a 5-membered cycloalkyl or heterocyclyl.

25. The compound of claim 24, wherein Ring A is cyclopentyl or tetrahydrofuranyl.

26. The compound of claim 25, wherein Ring A is 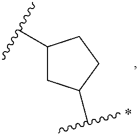 [SMILES: *C1CCC(*)C1]
wherein * denotes a bond to Ring B.

27. The compound of claim 1, wherein Ring A is a bridged bicyclic cycloalkyl or heterocyclyl.

28. The compound of claim 27, wherein Ring A is bicyclo[2.1.1]hexyl or oxabicyclo[2.1.1]hexyl.

29. The compound of claim 28, wherein Ring A is 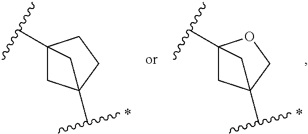 [SMILES: *C12CCC(*)(C1)C2 ; *C12COC(*)(C1)C2]
wherein * denotes a bond to Ring B.

30. The compound of claim 1, wherein Ring A is phenyl.

31. The compound of claim 30, wherein Ring A is 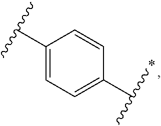 [SMILES: *c1ccc(*)cc1]
wherein * denotes a bond to Ring B.

32. The compound of claim 1, wherein Ring A is a polycyclic aryl, heteroaryl, cycloalkyl, or heterocyclyl.

33. The compound of claim 32, wherein Ring A is a bicyclic heteroaryl.

34. The compound of claim 33, wherein Ring A is 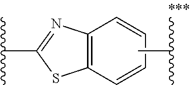
wherein *** denotes a —NH—R⁴.

35. The compound of claim 1, wherein Ring B is a 6-membered ring.

36. The compound of claim 35, wherein Ring B is phenyl.

37. The compound of claim 36, wherein Ring B is unsubstituted phenyl.

38. The compound of claim 36, wherein Ring B is a halogen-substituted phenyl.

39. The compound of claim 37, wherein Ring B is 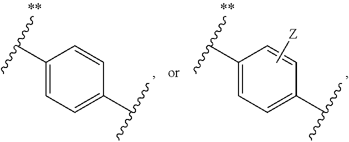
wherein Z is halo and ** denotes a bond to Ring A.

40. The compound of claim 35, wherein Ring B is a 6-membered heteroaryl.

41. The compound of claim 40, wherein Ring B is pyridinyl or pyrazinyl.

42. The compound of claim 41, wherein Ring B is 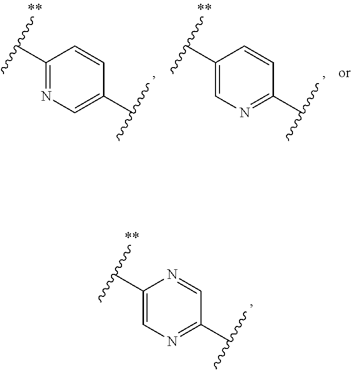 [SMILES: *c1ccc(*)nc1 ; *c1ccc(*)nc1 ; *c1cnc(*)cn1]
wherein ** denotes a bond to Ring A.

43. The compound of claim 1, wherein Ring B is a bridged bicyclic cycloalkyl.

44. The compound of claim 43, wherein Ring B is bicyclo[2.2.2]octanyl or bicyclo [1.1.1]pentanyl.

45. The compound of claim 44, wherein Ring B is 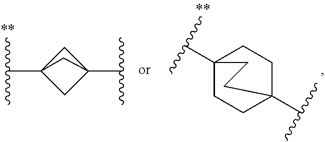
wherein Z is halo and ** denotes a bond to Ring A.

46. The compound of claim 1, wherein R⁴ is hydrogen, a protecting group, or —C(O)—CH₃.

47. The compound of claim 1 selected from: 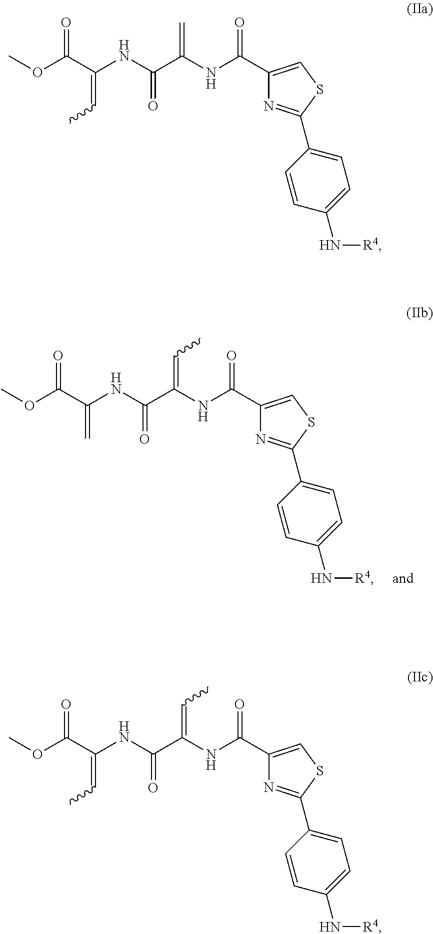
wherein R⁴ is hydrogen, a protecting group, or —C(O)—CH₃; and wherein a bond drawn as “ [symbol] ” denotes either possible stereochemistry of the attached alkene, E or Z.

48. The compound of claim 1 selected from 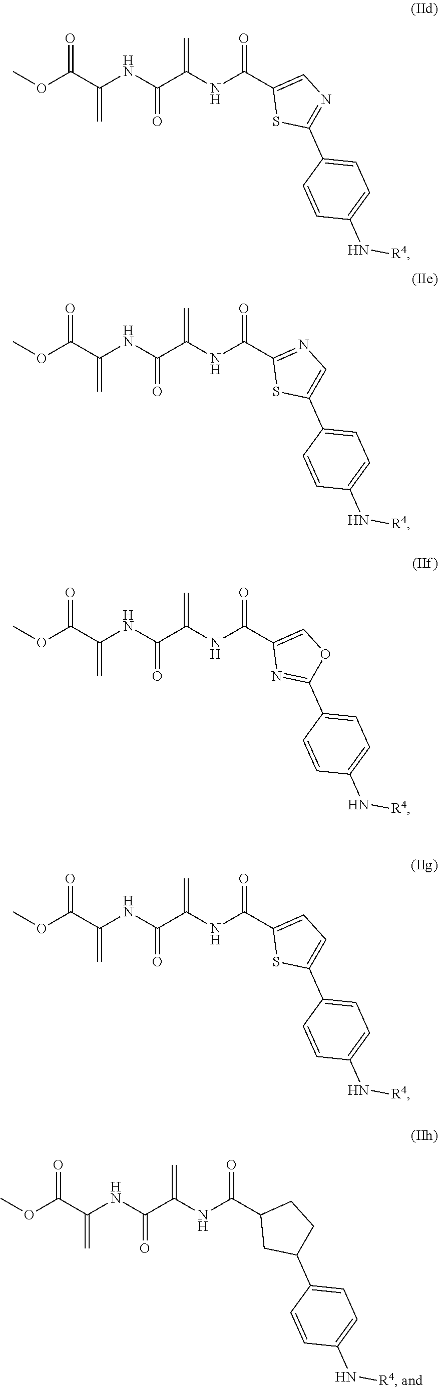 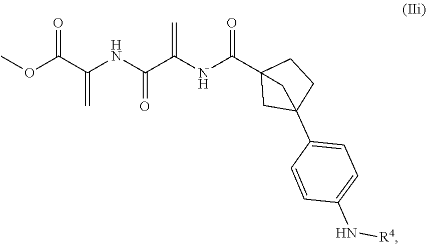
wherein R⁴ is hydrogen, a protecting group, or —C(O)—CH₃.

49. The compound of claim 1, selected from

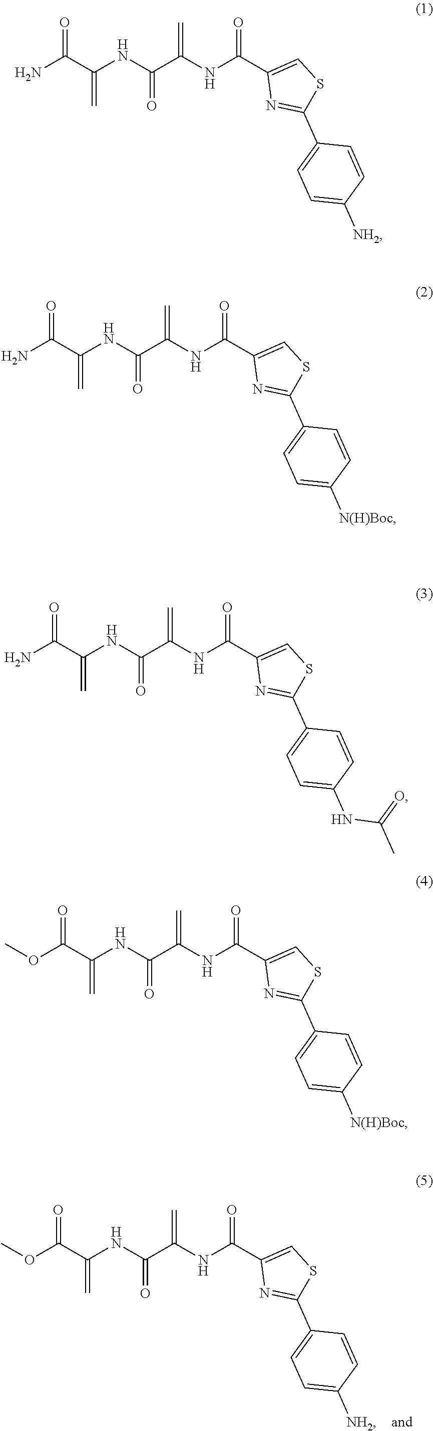

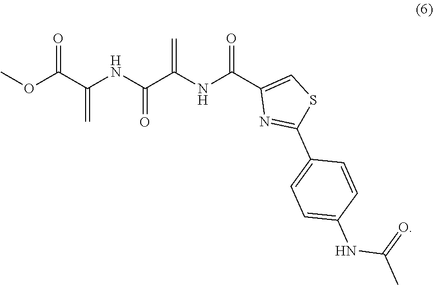 [SMILES: C=C(NC(=O)c1csc(-c2ccc(NC(C)=O)cc2)n1)C(=O)NC(=C)C(=O)OC]

50. The compound of claim 1, selected from 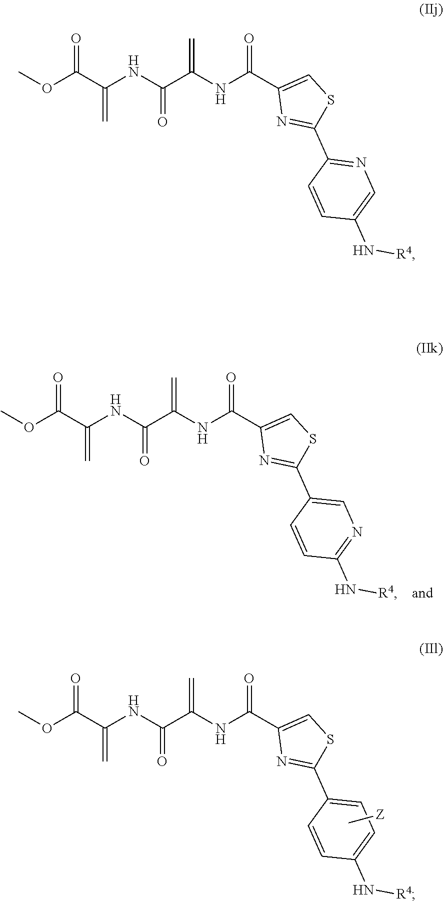
wherein R⁴ is hydrogen, a protecting group, or —C(O)—CH₃;
and wherein Z is selected from fluorine, chlorine, bromine, and iodine.

51. The compound of claim 1, selected from 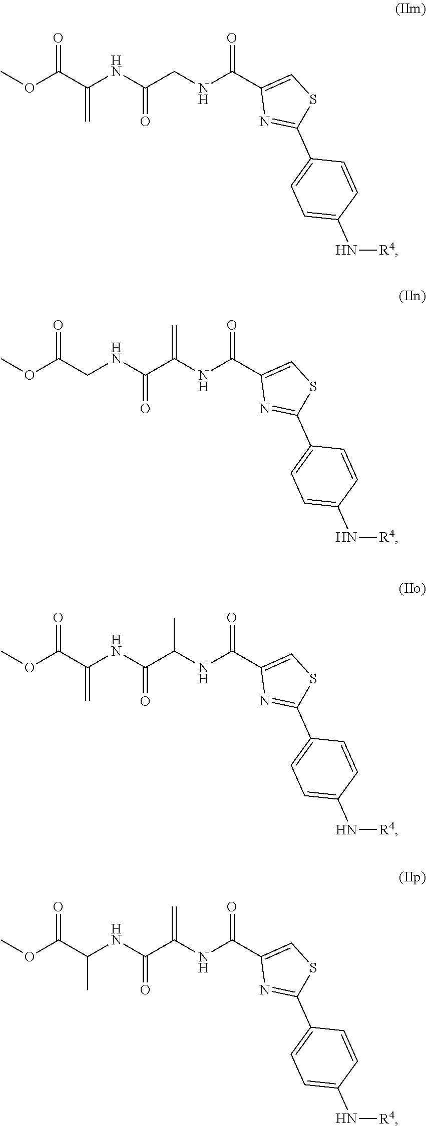 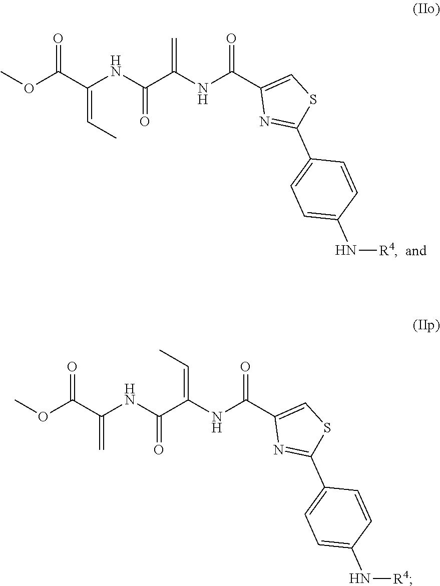
wherein R⁴ is hydrogen, a protecting group, or —C(O)—CH₃.

52. The compound of claim 51, wherein the protecting group is Boc.

53. The compound of claim 1, wherein R⁴ is —L′.

54. The compound of claim 53, wherein:
L′ is —C(O)—X—C(O)OH or —C(O)—X—C(O)NH₂,
X is —(CH₂)n—; and
n is 2, 3,4 or 5.

55. The compound of claim 54, selected from 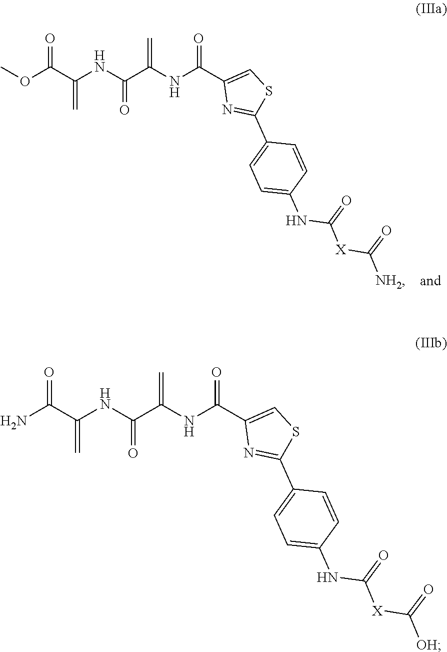
wherein X is —(CH₂)ₙ—; and n is 2, 3, 4 or 5.

56. The compound of claim 54, selected from 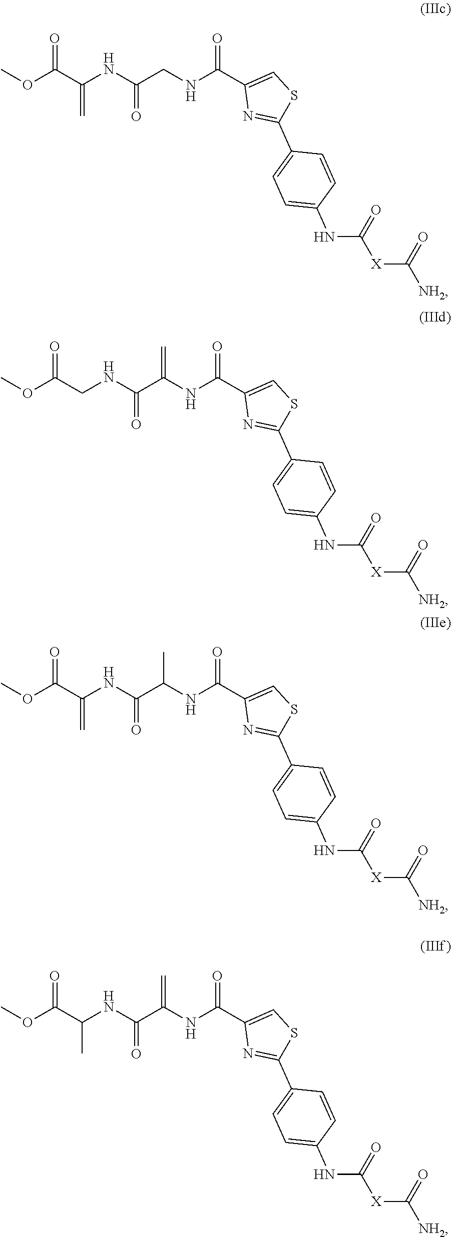 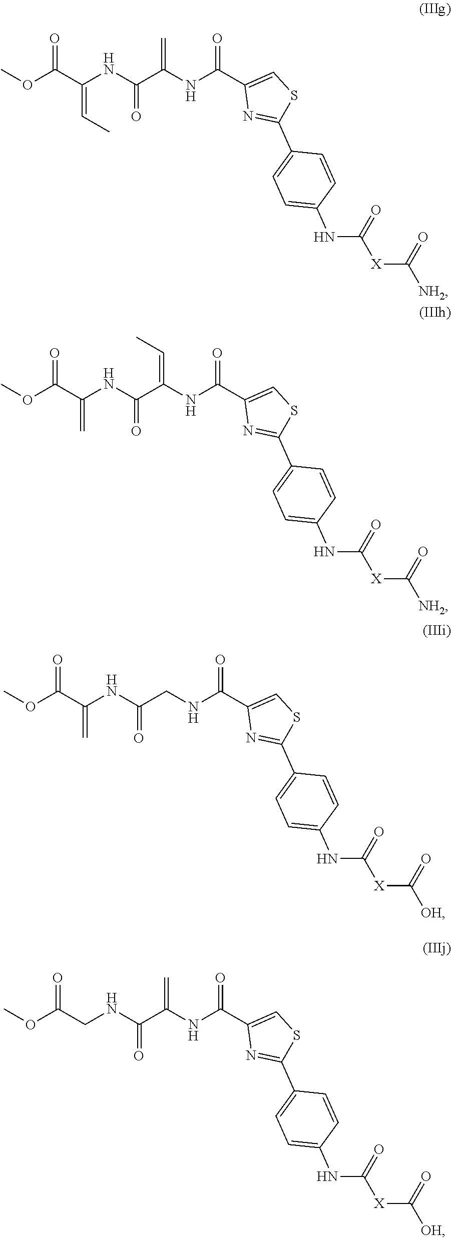 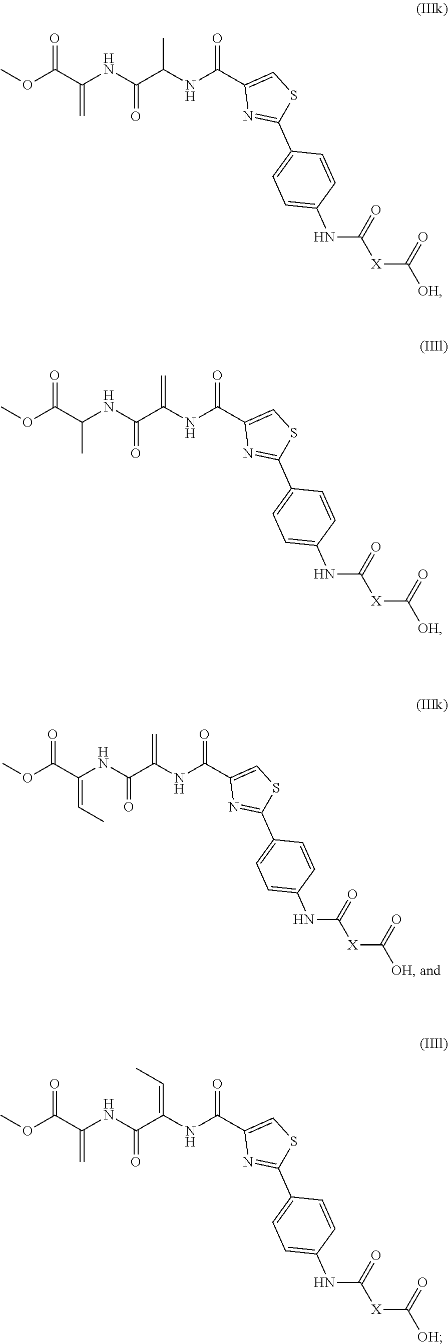
wherein X is —(CH₂)ₙ—; and
n is 2, 3,4 or 5.

57. The compound of claim 1, wherein R⁴ is —L—Y.

58. The compound of claim 57, wherein L is a cleavable linker.

59. The compound of claim 57, wherein L is a non-cleavable linker.

60. The compound of claim 57, wherein L has a chain length of about 2 to about 30 atoms.

61. The compound of claim 60, wherein L has a chain length of about 5 to about 20 atoms.

62. The compound of claim 57, wherein:
L is —C(O)—X—C(O)—;
X is —(CH₂)ₙ—; and
n is 2, 3,4 or 5.

63. The compound of claim 57, wherein:
L is —C(O)—X—C(O)—;
X is —(CH₂CH₂—O—)ₘ—(CH₂CH₂)—; and
m is 2, 3, 4, 5, or 6.

64. The compound of claim 1 selected from 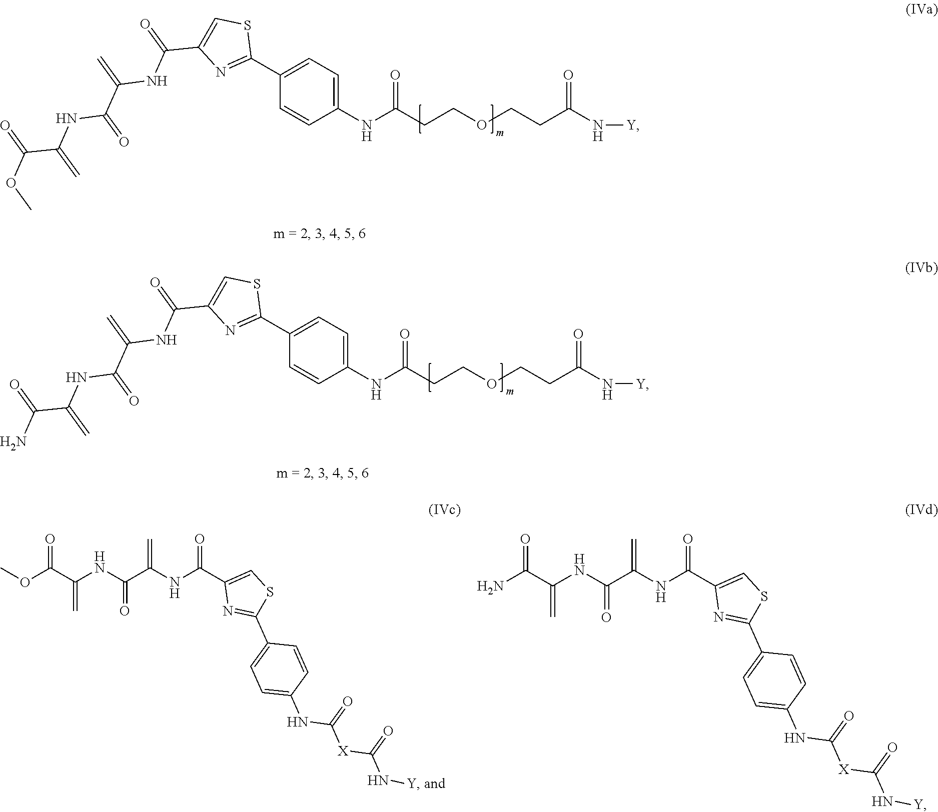
wherein X is —(CH₂)ₙ—; and
n is 2, 3, 4 or 5. 65.

65. The compound of claim 1 selected from 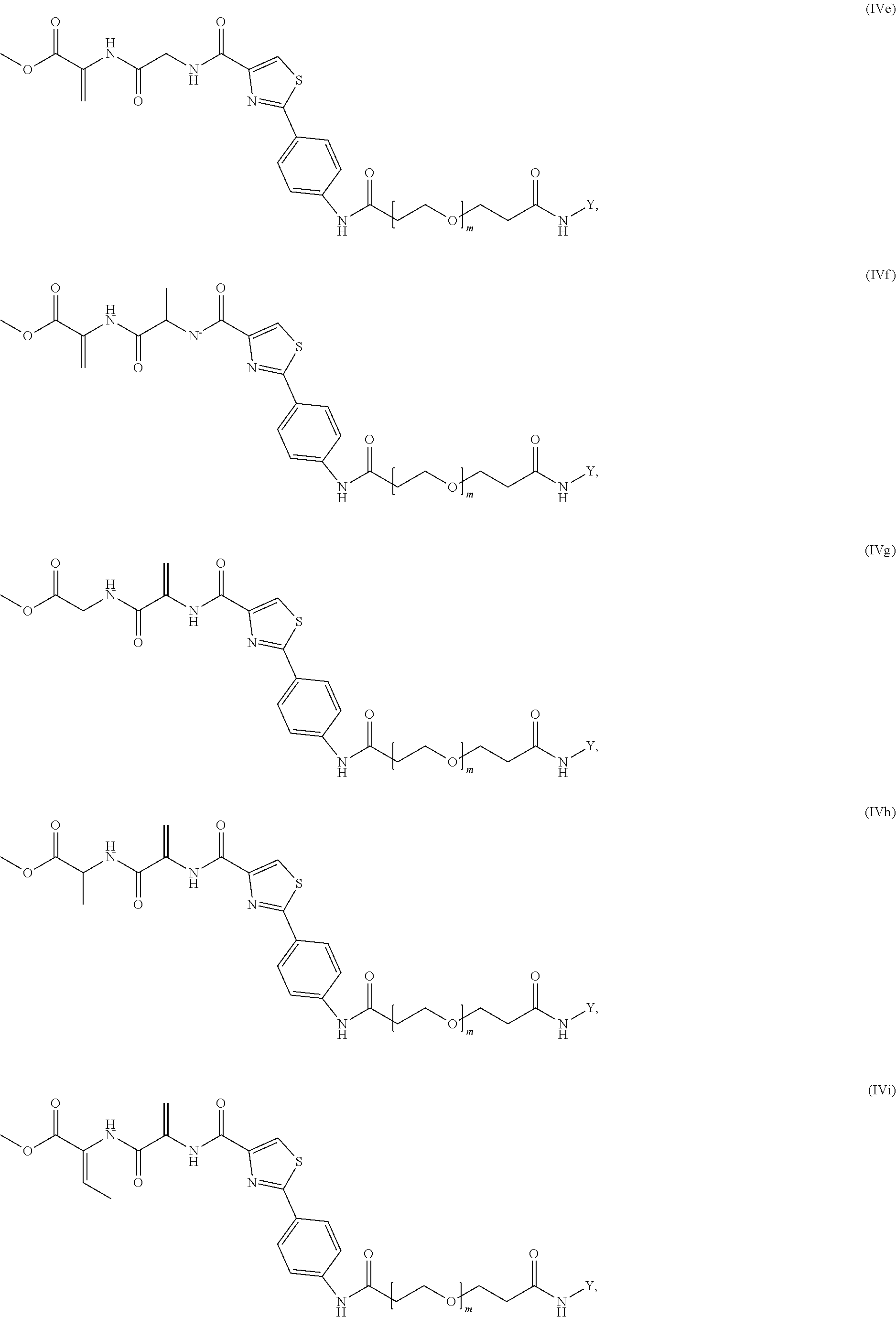 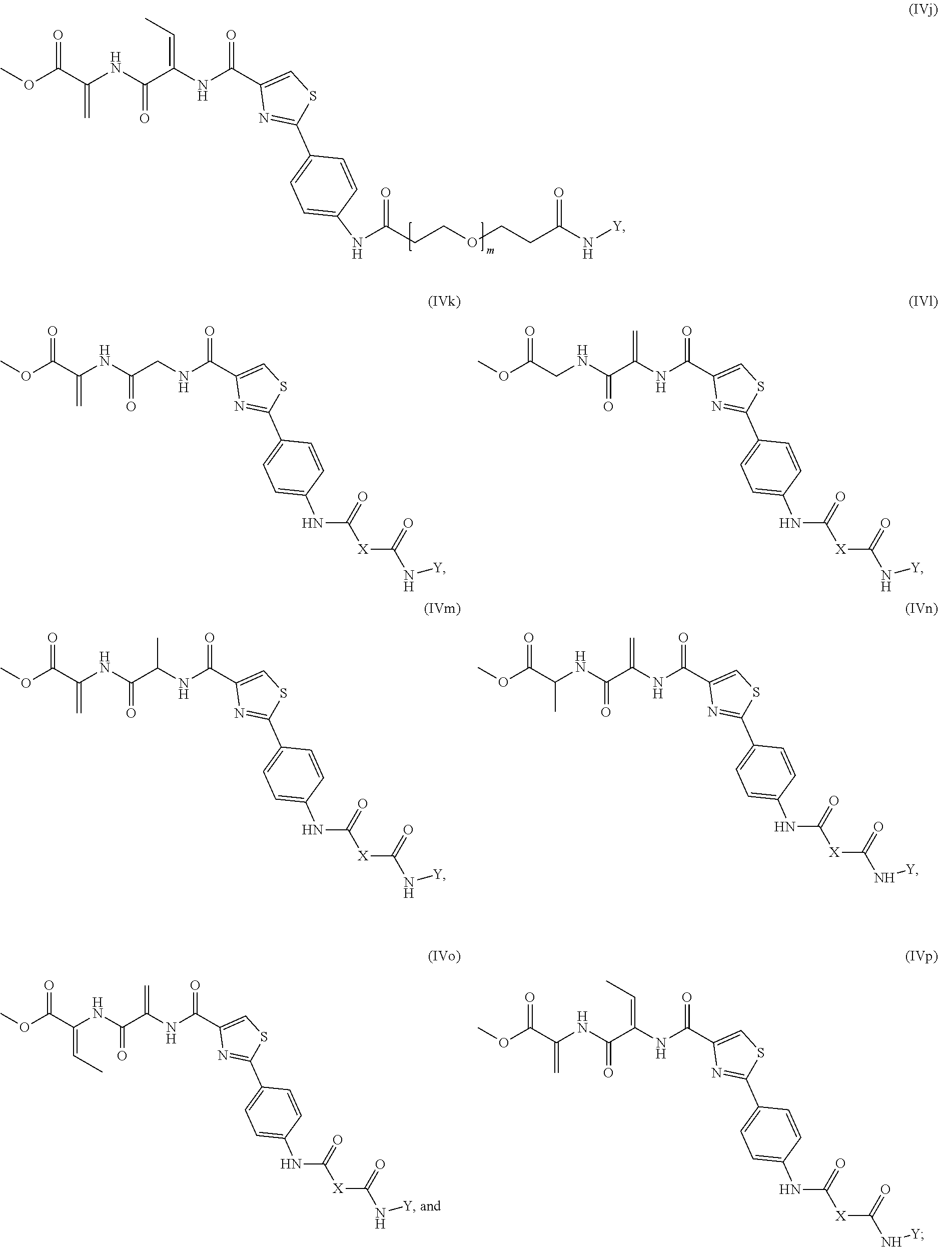
wherein X is —(CH₂)ₙ—;
m is 2, 3,4, 5, or 6; and
n is 2, 3,4 or 5.

66. The compound of claim 53, wherein L′ comprises an alkynyl or azido.

67. The compound of claim 66, wherein
R⁴ is —C(O)—X′—C═CH or —C(O)—X′—N₃;
X′ is —(CH₂)ₙ—; and
n is 2, 3,4 or 5.

68. The compound of claim 57, wherein L comprises a heteroaryl.

69. The compound of claim 68, wherein L comprises a triazolyl.

70. The compound of claim 69, wherein
R⁴ is 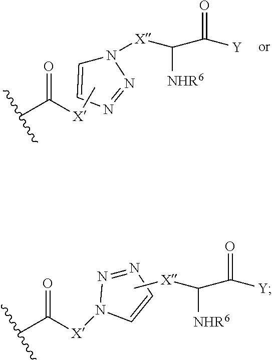
R⁶ is —H or —C(O)CH₃; X′ is —(CH₂)ₙ—;
X″ is —(CH₂)O—;
n is 2, 3, 4 or 5; and
o is 2, 3,4 or 5.

71. The compound of claim 70, wherein
R⁴is 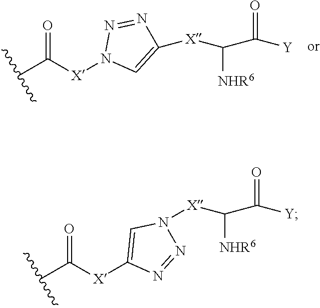
R⁶ is —H or —C(O)CH₃;
X′ is —(CH₂)ₙ—;
X″ is —(CH₂)ₒ—;
n is 2, 3, 4 or 5; and
o is 2, 3,4 or 5.

72. The compound of claim 69, wherein
R⁴ is 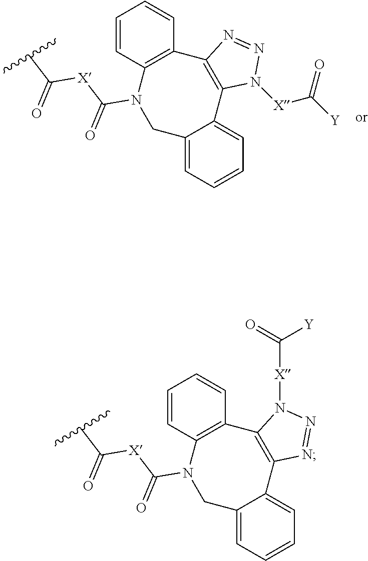
X′ is —(CH₂)ₙ—;
X″ is —(CH₂)_{O}—;
n is 2, 3, 4 or 5; and
o is 2, 3,4 or 5.

73. The compound of claim 57, wherein:
Y is a berberin cation, rhodamine cation, an indolium cation, a pyridinium cation, a tetraguanidinium cation, cyanine derivatives, a guanidinium cation, a biguanidinium cation, a triphenylphosphonium cation, a triethylammonium cation, a triphenylamine, a tetraphenyl ethene moiety, arylphosphonium cation, an SS peptide, a mitochondrial penetrating peptide (MPP), a mitochondrial targeting sequence (MTS) peptide, a hemigramicidin S-linked nitroxide, a Dequalinium (DQA) cation, a delocalized lipophilic cation, F16 ((E)-4-(1H-indol-3-ylvinyl)-N-methylpyridinium iodide), (L-cyclohexyl alanine-D-arginine)₃, a mitochondrial-targeted nanocarrier, a DDDK peptide, glycyrrhetinic acid, α-tocopheryl succinate (α-TOS), a graphene oxide nano carrier, PEG-proapoptotic peptide (KLAKLAK)₂, a Dmt-D-Arg-Phe-Lys-NH₂ peptide, pyruvaldehyde, N-Nonyl acridine orange, quinoline, styryl fluorophores, or 15d-PGJ2.

74. The compound of claim 73, wherein Y is a mitochondrial penetrating peptide (MPP).

75. The compound of claim 73, wherein Y has the structural formula (V)

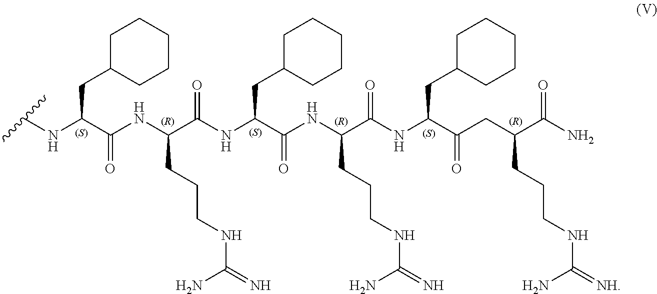

76. The compound of claim 1, selected from 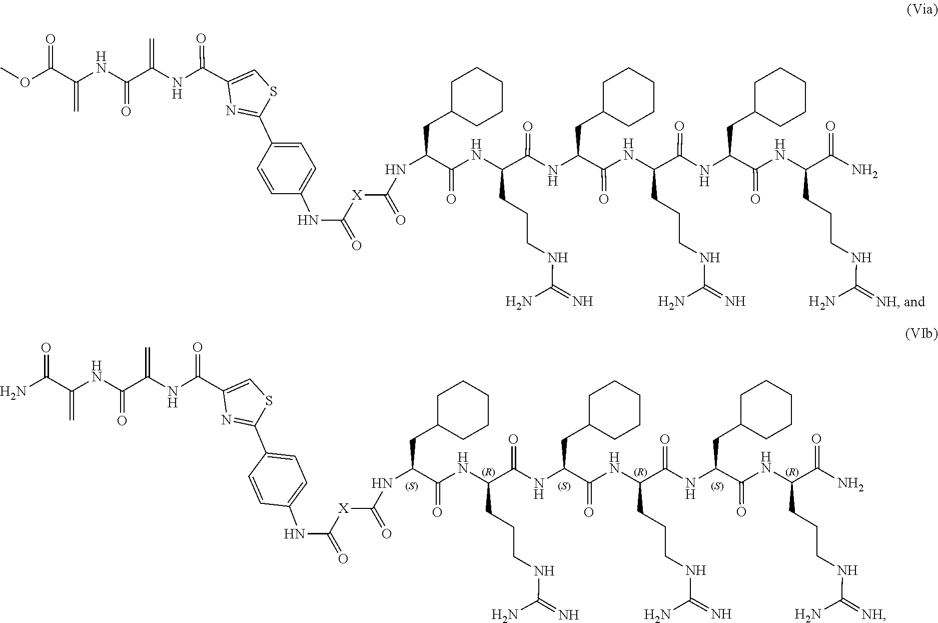
or a pharmaceutically acceptable salt thereof;
wherein X is [—(CH₂)ₙ-] or [—(CH₂CH₂—O—)ₘ—(CH₂CH₂)—];
n is 3, 4 or 5; and
m is 2, 3, 4, 5, or 6.

77. A pharmaceutically acceptable composition comprising a compound of claim 1; and a pharmaceutically acceptable carrier.

78. The composition of claim 77, formulated for oral or parenteral delivery.

79. A composition comprising a compound of claim 1, wherein the compound is contained within a nanoparticle, liposome or micelle, wherein the nanoparticle, liposome, or micelle is conjugated to a mitochondrial targeting moiety.

80. A composition comprising a compound of Formula (IA), wherein R⁴ is hydrogen, a protecting group, or —C(O)—CH₃, wherein the compound is contained within a nanoparticle, liposome or micelle, wherein the nanoparticle, liposome, or micelle is conjugated to a mitochondrial targeting moiety.

81. A composition comprising a compound of Formula (IB), wherein R⁴ is hydrogen, a protecting group, or —C(O)—CH₃, wherein the compound is contained within a nanoparticle, liposome or micelle, wherein the nanoparticle, liposome, or micelle is conjugated to a mitochondrial targeting moiety.

82. The composition of claim 79, wherein the nanoparticle, liposome or micelle is selected from polyethylene glycol), poly(ε-caprolactone), polysaccharides, poly[(2-hydroxypropyl)-methacrylic acid], poly(lactic-co-glycolic acid), and any combinations of the foregoing.

83. A method of treating a cancer (e.g., solid tumor or hematological cancer) comprising administering to a subject in need thereof a therapeutically effective amount of a compound of claim 1.

84. The method of claim 83, wherein the cancer (solid tumor or hematological) is selected from lung, breast, prostate, melanoma, esophageal, leukemia, cervical, liver, colon, gastric, colorectal, glioblastoma, head and neck, pancreatic, mesothelioma, and ovarian.

85. The method of claim 84, wherein the cancer is selected from mesothelioma, lung, ovarian, and breast.
